(19) United States
(12) Patent Application Publication
(10) Pub. No.: US 2026/0133491 A1
(43) Pub. Date: May 14, 2026

(54) ONIUM SALT MONOMER, POLYMER, CHEMICALLY AMPLIFIED RESIST COMPOSITION, AND PATTERN FORMING PROCESS

(71) Applicant: Shin-Etsu Chemical Co., Ltd., Tokyo (JP)

(72) Inventor: Masahiro Fukushima, Joetsu-shi (JP)

(73) Assignee: Shin-Etsu Chemical Co., Ltd., Tokyo (JP)

(21) Appl. No.: 19/377,159

(22) Filed: Nov. 3, 2025

(30) Foreign Application Priority Data
Nov. 11, 2024 (JP) 2024-196586

(51) Int. Cl. G03F 7/039; C07C 381/12; C08F 20/38; G03F 7/00

(52) U.S. Cl. CPC G03F 7/0397; C07C 381/12; C08F 20/38; G03F 7/0392; G03F 7/70033

(57) ABSTRACT

An onium salt monomer containing an aromatic sulfonic acid anion having a partial structure of a fused ring aromatic compound, specifically acenaphthylene is provided. A chemically amplified resist composition comprising a polymer comprising repeat units derived from the onium salt monomer has satisfactory solvent solubility, high sensitivity, and high contrast, and forms a resist film with improved lithography properties including LWR, CDU, EL and DOF.

### CROSS-REFERENCE TO RELATED APPLICATION

This non-provisional application claims priority under 35 U.S.C. § 119 (a) on Patent Application No. 2024-196586 filed in Japan on Nov. 11, 2024, the entire contents of which are hereby incorporated by reference.

### TECHNICAL FIELD

This invention relates to an onium salt monomer, polymer, chemically amplified resist composition, and pattern forming process.

### BACKGROUND ART

To meet the demand for higher integration density and operating speed of LSIs, the effort to reduce the pattern rule is in rapid progress. The wide-spreading flash memory market and the demand for increased storage capacities drive forward the miniaturization technology. As the advanced miniaturization technology, manufacturing of microelectronic devices at the 65-nm node by the ArF lithography has been implemented in a mass scale. Manufacturing of 45-nm node devices by the next generation ArF immersion lithography is approaching to the verge of high-volume application. The candidates for the next generation 32-nm node include ultra-high NA lens immersion lithography using a liquid having a higher refractive index than water in combination with a high refractive index lens and a high refractive index resist film, EUV lithography of wavelength 13.5 nm, and double patterning version of the ArF lithography, on which active research efforts have been made.

As the pattern feature size is reduced, approaching to the diffraction limit of light, light contrast lowers. In the case of positive resist film, a lowering of light contrast leads to reductions of resolution and focus margin of hole and trench patterns.

As the pattern feature size is reduced, the line width roughness (LWR) of line patterns and the critical dimension uniformity (CDU) of hole patterns are regarded significant. It is pointed out that these factors are affected by the segregation or agglomeration of a base polymer and acid generator and the diffusion of generated acid. There is a tendency that as the resist film becomes thinner, LWR becomes greater. A film thickness reduction to comply with the progress of size reduction causes a degradation of LWR, which becomes a serious problem.

The EUV lithography resist must meet high sensitivity, high resolution and low LWR at the same time. As the acid diffusion distance is reduced, LWR is reduced, but sensitivity becomes lower. For example, as the PEB temperature is lowered, the outcome is a reduced LWR, but a lower sensitivity. As the amount of quencher added is increased, the outcome is a reduced LWR, but a lower sensitivity. It is necessary to overcome the tradeoff relation between sensitivity and LWR.

With the aim to suppress acid diffusion, Patent Document 1 discloses a resist compound comprising repeat units derived from an onium salt of a polymerizable unsaturated bond-containing sulfonic acid. Since the so-called polymer-bound acid generator is capable of generating a polymer type sulfonic acid upon exposure, it is characterized by a very short distance of acid diffusion. Sensitivity may be enhanced by increasing a proportion of the acid generator. In the case of addition type acid generators, as the amount of acid generator added is increased, a higher sensitivity is achievable, but the acid diffusion distance is also increased. Since the acid diffusion is non-uniform, an increase of acid diffusion leads to degraded LWR or CDU. With respect to a balance of sensitivity, LWR and CDU, the polymer-bound acid generator is regarded as having a high capability.

Since iodine atoms are highly absorptive to EUV of wavelength 13.5 nm, they generate secondary electrons upon light exposure. This effect is noteworthy in the EUV lithography. Patent Document 2 describes a photoacid generator having an iodized anion. Patent Document 3 describes a photoacid generator having an iodized anion and containing a polymerizable group. Although the lithography performance is improved to some extent, the organic solvent solubility of iodine-containing compounds is not so high, accompanied with a concern about precipitation in the solvent.

Patent Documents 4 and 5 disclose an onium salt monomer having a polymerizable group of acenaphthylene or maleimide and capable of generating a fluoroalkanesulfonic acid. Patent Document 6 discloses an onium salt monomer capable of generating an acenaphthylene or indene sulfonic acid. Although the lithography performance is improved to a certain extent, there is left room for further improvement. It is desired to have a resist material which is useful in forming small-size patterns.

### CITATION LIST

Patent Document 1: JP 4425776

Patent Document 2: JP 6720926

Patent Document 3: JP 6973274

Patent Document 4: JP-A 2024-137079

Patent Document 5: JP-A 2024-112755

Patent Document 6: WO 2022/172689

### SUMMARY OF THE INVENTION

It is desired to develop an acid-catalyzed or chemically amplified resist composition exhibiting a high sensitivity and improved lithography properties including LWR, CDU, EL, and DOF.

An object of the invention is to provide a sulfonium salt monomer, a polymer comprising repeat units derived from the monomer, and a chemically amplified resist composition comprising the polymer, the resist composition, when processed by photolithography using high-energy radiation such as KrF excimer laser, ArF excimer laser, EB or EUV, exhibiting a satisfactory solvent solubility, high sensitivity, high contrast, and improved lithography properties including LWR, CDU, EL, and DOF. Another object of the invention is to provide a pattern forming process using the resist composition.

The inventor has found that using a polymer comprising repeat units derived from an onium salt containing an aromatic sulfonic acid anion having a partial structure of a fused ring aromatic compound, specifically acenaphthylene, there is obtained a chemically amplified resist composition having a high sensitivity, high contrast, high resolution, and improved lithography properties including LWR, CDU, EL and DOF by virtue of substantially restrained acid diffusion as well as etching resistance.

In one aspect, the invention provides an onium salt monomer having the formula (A).

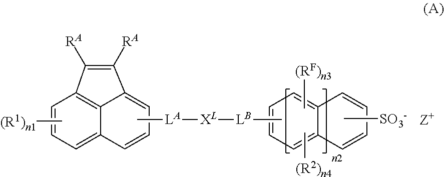

Herein n1 is 0, 1, 2, 3 or 4, n2 is 0 or 1, n3 is 0, 1, 2, 3 or 4, n4 is 0, 1, 2, 3 or 4, meeting 0≤n3+n4≤4 when n2-0 and 0≤n3+n4≤6 when n2=1,
R^{A} is each independently hydrogen, fluorine, methyl or trifluoromethyl,
R¹ is halogen, hydroxy, nitro, cyano, a C₁-C₂₀ hydrocarbyl group which may contain a heteroatom, a C₁-C₂₀ hydrocarbyloxy group which may contain a heteroatom, or a C₁-C₂₀ hydrocarbylthio group which may contain a heteroatom, when n1 is 2, 3 or 4, a plurality of R¹ may be identical or different and may bond together to form a ring with the carbon atom to which they are attached,
R² is halogen exclusive of fluorine, nitro, hydroxy, a C₁-C₂₀ hydrocarbyl group which may contain a heteroatom exclusive of fluorine, a C₁-C₂₀ hydrocarbyloxy group which may contain a heteroatom exclusive of fluorine, or a C₁-C₂₀ hydrocarbylthio group which may contain a heteroatom exclusive of fluorine; when n4 is 2, 3 or 4, a plurality of R² may be identical or different and may bond together to form a ring with the carbon atoms to which they are attached,
R^{F} is fluorine, a C₁-C₆ fluorinated saturated hydrocarbyl group, a C₁-C₆ fluorinated saturated hydrocarbyloxy group, or a C₁-C₆ fluorinated saturated hydrocarbylthio group, when n3 is 2, 3 or 4, a plurality of R^{F} may be identical or different,
L^{A} and L^{B} are each independently a single bond, ether bond, ester bond, amide bond, sulfonate ester bond, sulfonamide bond, carbonate bond or carbamate bond,
X^{L1} is a single bond or a C₁-C₄₀ hydrocarbylene group which may contain a heteroatom, and
Z⁺ is an onium cation.

The preferred onium salt monomer has the formula (A1):

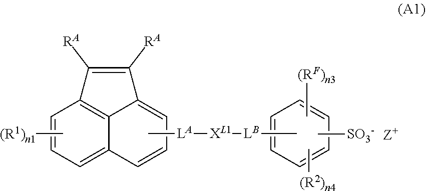

wherein n1, n3, n4, R^{A}, R¹, R², R^{F}, L^{A}, L^{B}, X^{L1}, and Z⁺ are as defined above.

The more preferred onium salt monomer has the formula (A2):

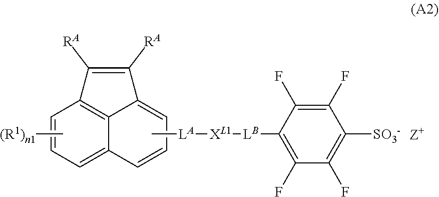

wherein n1, R^{A}, R¹, L^{A}, L^{B}, X^{L1}, and Z⁺ are as defined above.

In a preferred embodiment, Z⁺ is a sulfonium cation having the formula (Z-1) or iodonium cation having the formula (Z-2):

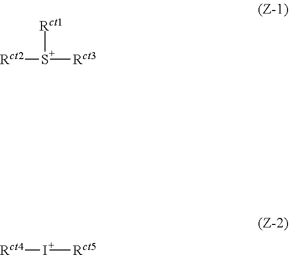

wherein R^{ct1} to R^{ct5} are each independently halogen or a C₁-C₃₀ hydrocarbyl group which may contain a heteroatom, and R^{ct1} and R^{ct2} may bond together to form a ring with the sulfur atom to which they are attached.

In another preferred embodiment, Z⁺ is a sulfonium cation having the formula (Z-3).

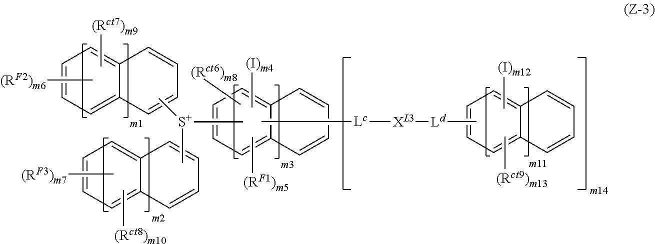

Herein m1 is 0 or 1, m2 is 0 or 1, m3 is 0 or 1, m4 is 0, 1, 2, 3 or 4, m5 is 0, 1, 2, 3 or 4, m6 is 0, 1, 2, 3, 4, 5 or 6, m7 is 0, 1, 2, 3, 4, 5 or 6, m8 is 0, 1 or 2, m9 is 0, 1 or 2, m10 is 0, 1 or 2, m11 is 0 or 1, m12 is 0, 1, 2, 3 or 4, m13 is 0, 1 or 2, m14 is 0, 1 or 2, meeting 0≤m6+m9≤4 when m1=0, 0≤m6+m9≤6 when m1=1, 0≤m7+m10≤4 when m2=0, 0≤m7+m10≤6 when m2=1, 1≤m4+m5+m8+m14≤4 when m3=0, 1≤m4+m5+m8+m14≤6 when m3=1, 0≤m12+m13≤4 when m11=0, 0≤m12+m13≤6 when m11=1, and m4+m12≥1,
R^{F1} to R^{F3} are each independently fluorine, a C₁-C₆ fluorinated saturated hydrocarbyl group, C₁-C₆ fluorinated saturated hydrocarbyloxy group, or C₁-C₆ fluorinated saturated hydrocarbylthio group, with the proviso that a plurality of R^{F1} may be identical or different when m5 is 2, 3 or 4, a plurality of R^{F2} may be identical or different when m6 is 2, 3, 4, 5 or 6, and a plurality of R^{F3} may be identical or different when m7 is 2, 3, 4, 5 or 6,
R^{ct6} to R^{ct9} are each independently halogen exclusive of iodine and fluorine, nitro, cyano, a C₁-C₂₀ hydrocarbyl group which may contain a heteroatom, C₁-C₂₀ hydrocarbyloxy group which may contain a heteroatom, or C₁-C₂₀ hydrocarbylthio group which may contain a heteroatom, with the proviso that two R^{ct6} may be identical or different and two R^{ct6} may bond together to form a ring with the carbon atoms to which they are attached when m8-2, two R^{ct7} may be identical or different and two R^{ct7} may bond together to form a ring with the carbon atoms to which they are attached when m9=2, two R^{ct8} may be identical or different and two R^{ct8} may bond together to form a ring with the carbon atoms to which they are attached when m10=2, two R^{ct9} may be identical or different and two R^{ct9} may bond together to form a ring with the carbon atoms to which they are attached when m13=2,
the aromatic rings directly bonded to S⁺ in the sulfonium cation may bond together to form a ring with S⁺,
L^{C} and L^{D} are each independently a single bond, ether bond, ester bond, sulfonate ester bond, amide bond, sulfonamide bond, carbonate bond or carbamate bond, and
X^{L2} is a single bond or a C₁-C₄₀ hydrocarbylene group which may contain a heteroatom.

In another aspect, the invention provides a polymer comprising repeat units derived from the onium salt monomer defined herein.

The polymer functions as a polymer-bound acid generator.

The polymer may further comprise repeat units of at least one type selected from repeat units having the formula (a1), repeat units having the formula (a2), and repeat units having the formula (a3).

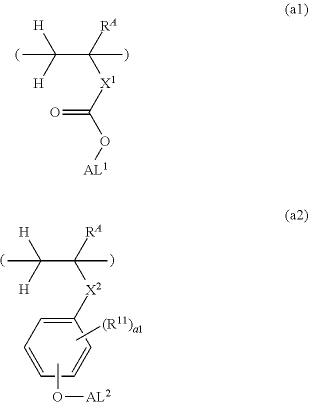

Herein R^{A} is each independently hydrogen, fluorine, methyl or trifluoromethyl,
X¹ is a single bond, phenylene group, naphthylene group, *—C(═O)—O—X¹¹—, or *—C(═O)—NH—X¹¹—, the phenylene and naphthylene groups may be substituted with hydroxy, nitro, cyano, a C₁-C₁₀ saturated hydrocarbyl moiety which may contain fluorine, C₁-C₁₀ saturated hydrocarbyloxy moiety which may contain fluorine, or halogen, X¹¹ is a C₁-C₁₀ saturated hydrocarbylene group, phenylene group or naphthylene group, the saturated hydrocarbylene group may contain hydroxy, ether bond, ester bond or lactone ring,
X² is a single bond, *—C(═O)—O—, or *—C(═O)—NH—,
* designates a point of attachment to the carbon atom in the backbone,
R¹¹ is halogen, cyano, hydroxy, nitro, a C₁-C₂₀ hydrocarbyl group which may contain a heteroatom, C₁-C₂₀ hydrocarbyloxy group which may contain a heteroatom, C₂-C₂₀ hydrocarbylcarbonyl group which may contain a heteroatom, C₂-C₂₀ hydrocarbylcarbonyloxy group which may contain a heteroatom, or C₂-C₂₀ hydrocarbyloxycarbonyl group which may contain a heteroatom, and when a1 is 2, 3 or 4, a plurality of R¹¹ may be identical or different,
AL¹ and AL² are each independently an acid labile group, and
a1 is 0, 1, 2, 3 or 4,

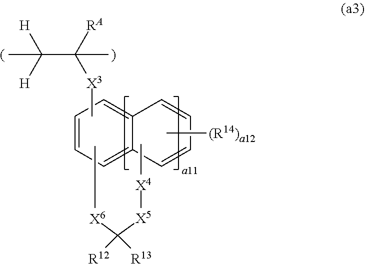

wherein all is 0 or 1, a12 is 0, 1, 2 or 3 when a11=0, a12 is 0, 1, 2, 3, 4 or 5 when a11=1,
R^{A} is hydrogen, fluorine, methyl or trifluoromethyl,
X³ is a single bond, *—C(═O)—O— or *—C(═O)—NH—, * designates a point of attachment to the carbon atom in the backbone,
X⁴ is a single bond, C₁-C₄ aliphatic hydrocarbylene group, carbonyl group, sulfonyl group or a combination thereof,
X⁵ and X⁶ are each independently oxygen or sulfur, with the proviso that X⁴ and X⁶ are attached to adjoining carbon atoms on the aromatic ring,
R¹² and R¹³ are each independently hydrogen or a C₁-C₂₀ hydrocarbyl group which may contain a heteroatom, R¹² and R¹³ may bond together to form a ring with the carbon atom to which they are attached,
R¹⁴ is halogen, hydroxy, cyano, nitro, a C₁-C₂₀ hydrocarbyl group which may contain a heteroatom, C₁-C₂₀ hydrocarbyloxy group which may contain a heteroatom, C₂-C₂₀ hydrocarbyloxycarbonyl group which may contain a heteroatom, C₁-C₂₀ hydrocarbylthio group which may contain a heteroatom, or —N(R^{14A})(R^{14B}), R^{14A} and R^{14B} are each independently hydrogen or a C₁-C₆ hydrocarbyl group, and when a12 is 2 or more, a plurality of R¹⁴ may be identical or different and a plurality of R¹⁴ may bond together to form a ring with the carbon atoms on the aromatic ring to which they are attached.

The polymer may further comprise repeat units of at least one type selected from repeat units having the formula (b1) and repeat units having the formula (b2).

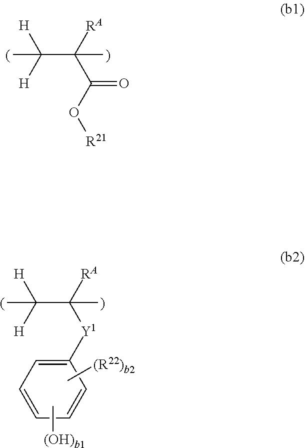

Herein R^{A} is each independently hydrogen, fluorine, methyl or trifluoromethyl,
Y¹ is a single bond or *—C(═O)—O—, * designates a point of attachment to the carbon atom in the backbone,
R²¹ is hydrogen or a C₁-C₂₀ group containing at least one structure selected from hydroxy other than phenolic hydroxy, cyano, carbonyl, carboxy, ether bond, ester bond, sulfonate ester bond, carbonate bond, lactone ring, sultone ring, and carboxylic anhydride (—C(═O)—O—C(═O)—),
R²² is halogen, carboxy, nitro, cyano, a C₁-C₂₀ hydrocarbyl group which may contain a heteroatom, C₁-C₂₀ hydrocarbyloxy group which may contain a heteroatom, C₂-C₂₀ hydrocarbylcarbonyl group which may contain a heteroatom, C₂-C₂₀ hydrocarbylcarbonyloxy group which may contain a heteroatom, or C₂-C₂₀ hydrocarbyloxycarbonyl group which may contain a heteroatom, and when b2 is 2, 3 or 4, a plurality of R²² may be identical or different,
b1 is 1, 2, 3 or 4, b2 is 0, 1, 2, 3 or 4, and 1≤b1+b2≤5.

In a further aspect, the invention provides a chemically amplified resist composition comprising (A) a base polymer containing the polymer defined herein.

The resist composition may further comprise (B) an organic solvent, (C) a quencher, (D) a photoacid generator, and/or (E) a surfactant.

In a still further aspect, the invention provides a pattern forming process comprising the steps of applying the chemically amplified resist composition defined herein onto a substrate to form a resist film thereon, exposing the resist film to high-energy radiation, and developing the exposed resist film in a developer.

Typically, the high-energy radiation is KrF excimer laser, ArF excimer laser, EB or EUV of wavelength 3 to 15 nm.

### Advantageous Effects of the Invention

When a chemically amplified resist composition comprising a polymer comprising repeat units derived from an onium salt monomer and functioning as a photoacid generator is processed by lithography, a resist pattern having a high sensitivity, high contrast and improved lithography properties including LWR, CDU, EL and DOF is formed.

### DESCRIPTION OF THE PREFERRED EMBODIMENT

As used herein, the singular forms “a,” “an” and “the” include plural referents unless the context clearly dictates otherwise. “Optional” or “optionally” means that the subsequently described event or circumstances may or may not occur, and that description includes instances where the event or circumstance occurs and instances where it does not. The notation (Cn-Cm) means a group containing from n to m carbon atoms per group. In chemical formulae, Me stands for methyl, Ac for acetyl. Both the broken line (---) and the asterisk (*) designate a point of attachment or valence bond. As used herein, the term “fluorinated” refers to a fluorine-substituted or fluorine-containing compound or group. The terms “group” and “moiety” are interchangeable.

The abbreviations and acronyms have the following meaning.
EB: electron beam
EUV: extreme ultraviolet
Mw: weight average molecular weight
Mn: number average molecular weight
Mw/Mn: molecular weight distribution or dispersity
GPC: gel permeation chromatography
PEB: post-exposure bake
PAG: photoacid generator
LWR: line width roughness
EL: exposure latitude
DOF: depth of focus
CDU: critical dimension uniformity

It is understood that for some structures represented by chemical formulae, there can exist enantiomers and diastereomers because of the presence of asymmetric carbon atoms. In such a case, a single formula collectively represents all such isomers. The isomers may be used alone or in admixture.

### [Onium Salt Monomer]

One embodiment of the invention is an onium salt monomer having the formula (A).

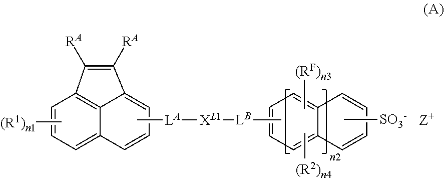

In formula (A), n1 is 0, 1, 2, 3 or 4, preferably 0, 1 or 2, more preferably 0 or 1. The subscript n2 is 0 or 1. The relevant structure is a benzene ring when n2=0 and a naphthalene ring when n2=1. From the aspect of solvent solubility, the benzene ring corresponding to n2-0 is preferred. The subscript n3 is 0, 1, 2, 3 or 4, and n4 is 0, 1, 2, 3 or 4. It is noted that n2 to n4 are in the range: 0≤n3+n4≤4 when n2=0 and 0≤n3+n4≤6 when n2=1.

In formula (A), R^{A} is each independently hydrogen, fluorine, methyl or trifluoromethyl, preferably hydrogen or methyl, most preferably hydrogen.

In formula (A), R¹ is halogen, hydroxy, nitro, cyano, a C₁-C₂₀ hydrocarbyl group which may contain a heteroatom, a C₁-C₂₀ hydrocarbyloxy group which may contain a heteroatom, or a C₁-C₂₀ hydrocarbylthio group which may contain a heteroatom. Suitable halogen atoms include fluorine, chlorine, bromine and iodine. The hydrocarbyl group and hydrocarbyl moiety in the hydrocarbyloxy and hydrocarbylthio groups may be saturated or unsaturated and straight, branched or cyclic. Examples thereof include C₁-C₂₀ alkyl groups such as methyl, ethyl, n-propyl, isopropyl, n-butyl, isobutyl, sec-butyl, tert-butyl, n-pentyl, n-hexyl, n-octyl, n-nonyl, n-decyl, undecyl, dodecyl, tridecyl, tetradecyl, pentadecyl, heptadecyl, octadecyl, nonadecyl, and icosyl: C₃-C₂₀ cyclic saturated hydrocarbyl groups such as cyclopropyl, cyclopentyl, cyclohexyl, cylopropylmethyl, 4-methylcyclohexyl, cyclohexylmethyl, norbomnyl, and adamantyl: C₂-C₂₀ alkenyl groups such as vinyl, 1-propenyl, 2-propenyl, butenyl, and hexenyl: C₃-C₂₀ cyclic unsaturated hydrocarbyl groups such as cyclohexenyl: C₆-C₂₀ aryl groups such as phenyl and naphthyl; C₇-C₂₀ aralkyl groups such as benzyl, 1-phenylethyl, and 2-phenylethyl, and combinations thereof. In the hydrocarbyl group, some or all of the hydrogen atoms may be substituted by a moiety containing a heteroatom such as oxygen, sulfur, nitrogen or halogen, and some constituent —CH₂— may be replaced by a moiety containing a heteroatom such as oxygen, sulfur or nitrogen, so that the group may contain a hydroxy moiety, cyano moiety, fluorine, chlorine, bromine, iodine, carbonyl moiety, ether bond, ester bond, sulfonate ester bond, carbonate bond, lactone ring, sultone ring, carboxylic anhydride (—C(═O)—O—C(═O)—) or haloalkyl moiety. When n1 is 2, 3 or 4, a plurality of R¹ may be identical or different.

When n1 is 2, 3 or 4, a plurality of R¹ may bond together to form a ring with the carbon atom to which they are attached. Examples of the ring include cyclopropane, cyclobutane, cyclopentane, cyclohexane, norbornane, and adamantane rings. In the ring, some or all of the hydrogen atoms may be substituted by a moiety containing a heteroatom such as oxygen, sulfur, nitrogen or halogen, and some constituent —CH₂— may be replaced by a moiety containing a heteroatom such as oxygen, sulfur or nitrogen, so that the ring may contain a hydroxy moiety, fluorine, chlorine, bromine, iodine, cyano moiety, carbonyl moiety, ether bond, ester bond, sulfonate ester bond, carbonate bond, lactone ring sultone ring, carboxylic anhydride (—C(═O)—O—C(═O)—) or haloalkyl moiety.

In formula (A), R² is halogen exclusive of fluorine, nitro, hydroxy, a C₁-C₂₀ hydrocarbyl group, C₁-C₂₀ hydrocarbyloxy group, or C₁-C₂₀ hydrocarbylthio group. The hydrocarbyl, hydrocarbyloxy and hydrocarbylthio groups may contain a heteroatom exclusive of fluorine. Suitable halogen atoms exclusive of fluorine include chlorine, bromine and iodine. The hydrocarbyl group or hydrocarbyl moiety in the hydrocarbyloxy and hydrocarbylthio groups may be saturated or unsaturated and straight, branched or cyclic. Examples thereof are as exemplified above for the hydrocarbyl group R¹, but not limited thereto. In the hydrocarbyl group, some or all of the hydrogen atoms may be substituted by a moiety containing a heteroatom such as oxygen, sulfur, nitrogen or halogen exclusive of fluorine, and some constituent —CH₂— may be replaced by a moiety containing a heteroatom such as oxygen, sulfur or nitrogen, so that the group may contain a hydroxy moiety, cyano moiety, chlorine, bromine, iodine, carbonyl moiety, ether bond, ester bond, sulfonate ester bond, carbonate bond, lactone ring, sultone ring, carboxylic anhydride (—C(═O)—O—C(═O)—) or haloalkyl moiety. When n4 is 2, 3 or 4, a plurality of R² may be identical or different.

When n4 is 2, 3 or 4, a plurality of R² may bond together to form a ring with the carbon atom to which they are attached. The ring is preferably 5 to 8-membered.

In formula (A), R^{F} is fluorine, a C₁-C₆ fluorinated saturated hydrocarbyl group, C₁-C₆ fluorinated saturated hydrocarbyloxy group or C₁-C₆ fluorinated saturated hydrocarbylthio group. Inter alia, fluorine, trifluoromethyl, trifluoromethoxy and trifluoromethylthio are preferred, with fluorine being more preferred. When n3 is 2, 3 or 4, a plurality of R^{F} may be identical or different.

In formula (A), L^{A} and L^{B} are each independently a single bond, ether bond, ester bond, sulfonate ester bond, amide bond, sulfonamide bond, carbonate bond or carbamate bond. Inter alia, a single bond, ether bond, ester bond, and sulfonate ester bond are preferred.

In formula (A), X^{L1} is a single bond or a C₁-C₄₀ hydrocarbylene group which may contain a heteroatom. The hydrocarbylene group may be straight, branched or cyclic. Examples thereof include alkanediyl and cyclic saturated hydrocarbylene groups. Suitable heteroatoms include oxygen, nitrogen, and sulfur.

Preferred examples of the C₁-C₄₀ hydrocarbylene group which may contain a heteroatom, represented by X^{L1}, are shown below. Herein asterisks (*) designate a point of attachment to L^{A} and L^{B}.

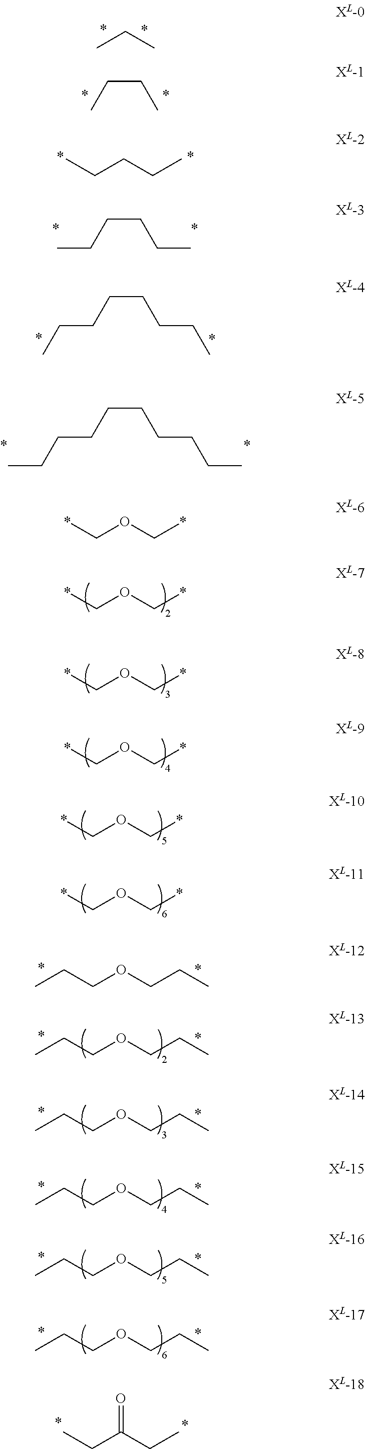

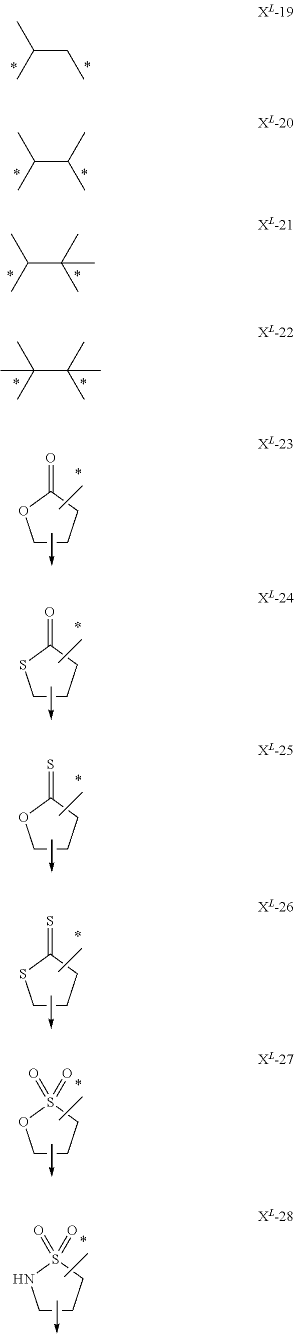

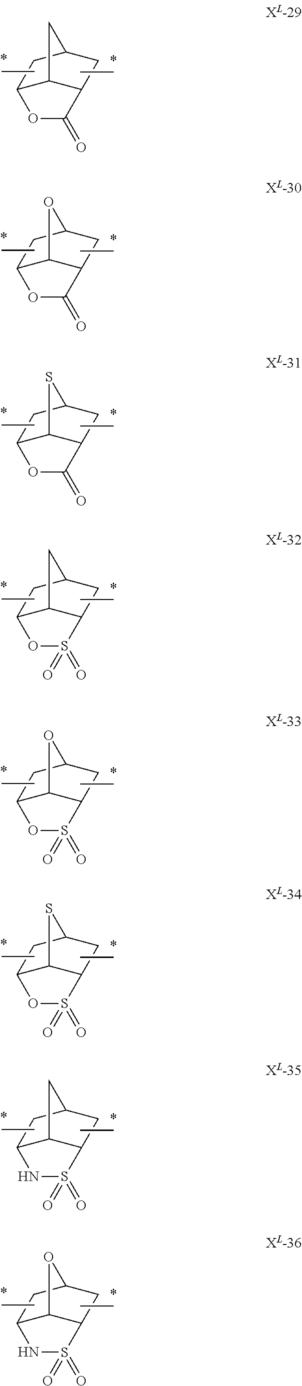

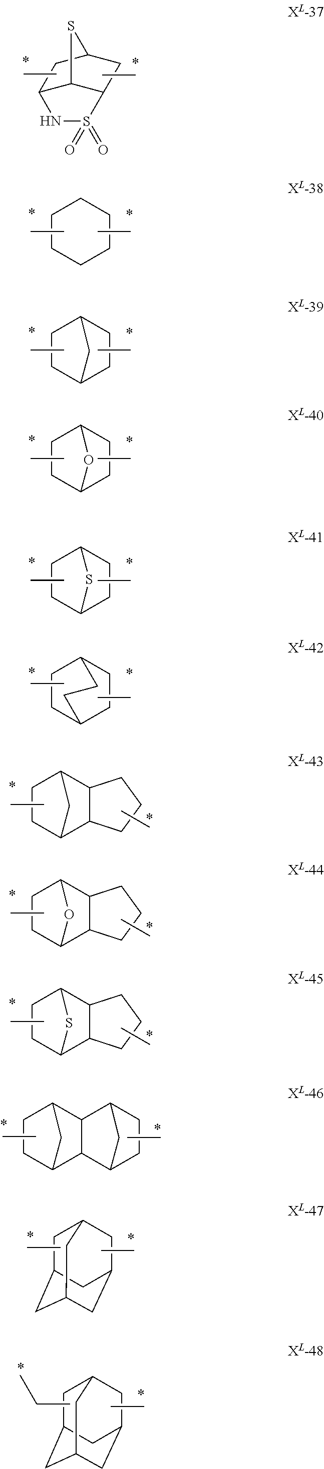

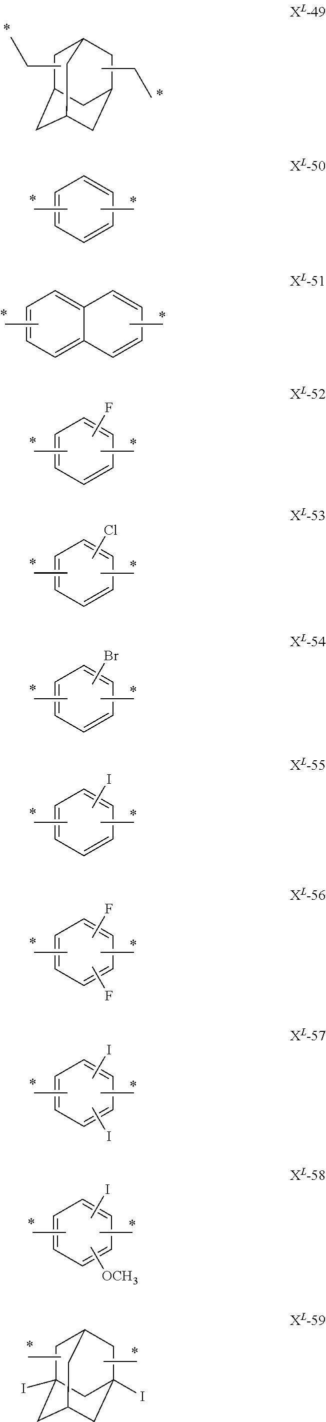

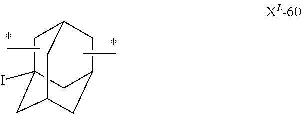

Of these. X^{L}-0 to X^{L}-22, X^{L}-29 to X^{L}-34, and X^{L}-47 to X^{L}-58 are preferred.

Of the onium salt monomers having formula (A), those having the formula (A1) are preferred.

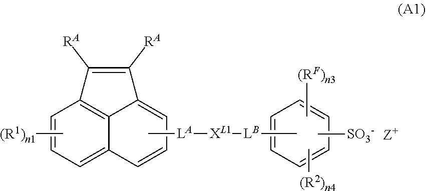

Herein n1, n3, n4, R^{A}, R¹, R², R^{F}, L^{A}, L^{B}, X^{L1}, and Z⁺ are as defined above.

Of the onium salt monomers having formula (A1), those having the formula (A2) are preferred.

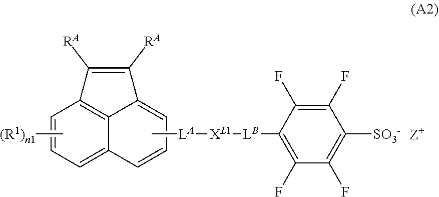

Herein n1, R^{A}, R¹, L^{A}, L^{B}, X^{L1}, and Z⁺ are as defined above.

Examples of the anion in the onium salt monomer having formula (A) are shown below, but not limited thereto. R^{A} is as defined above. The positions of attachment of substituent groups on the aromatic ring are interchangeable.

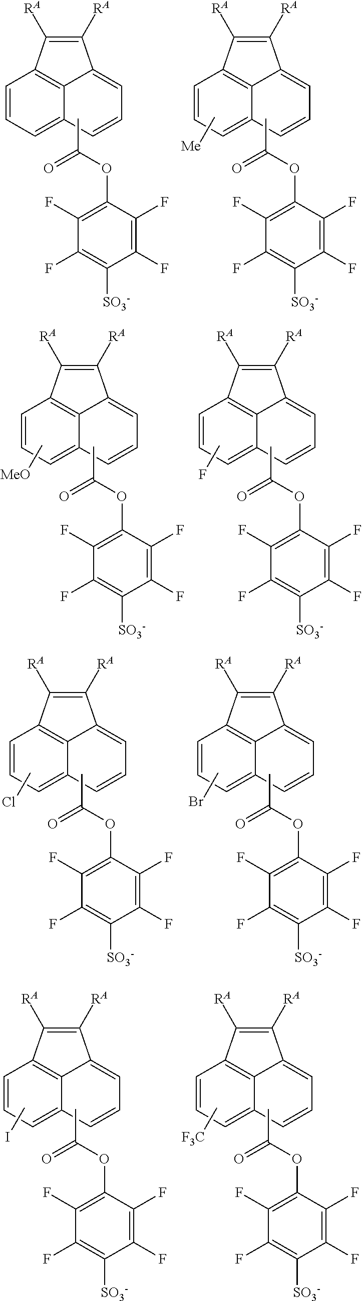

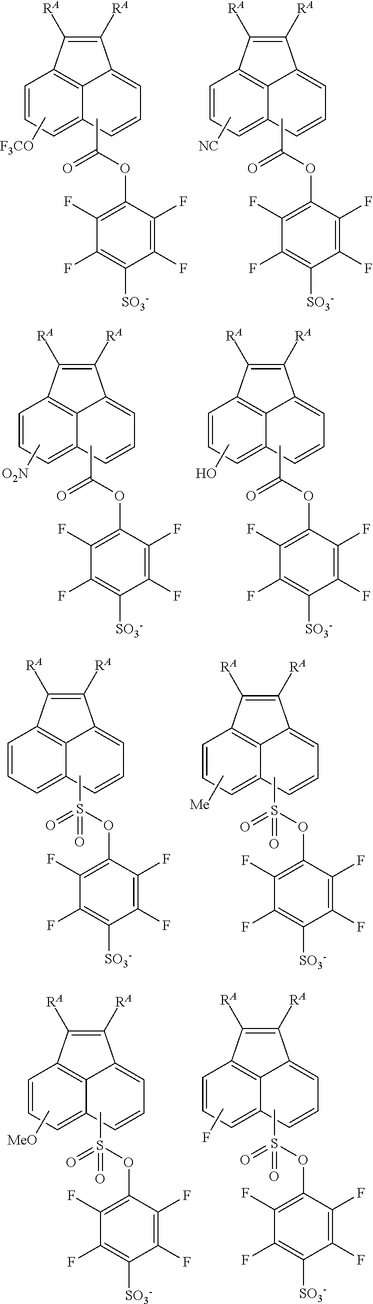

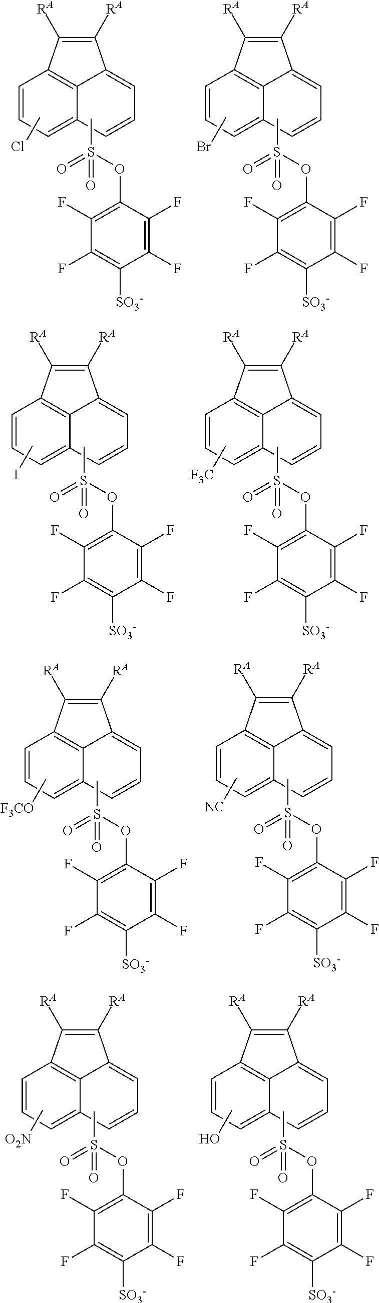

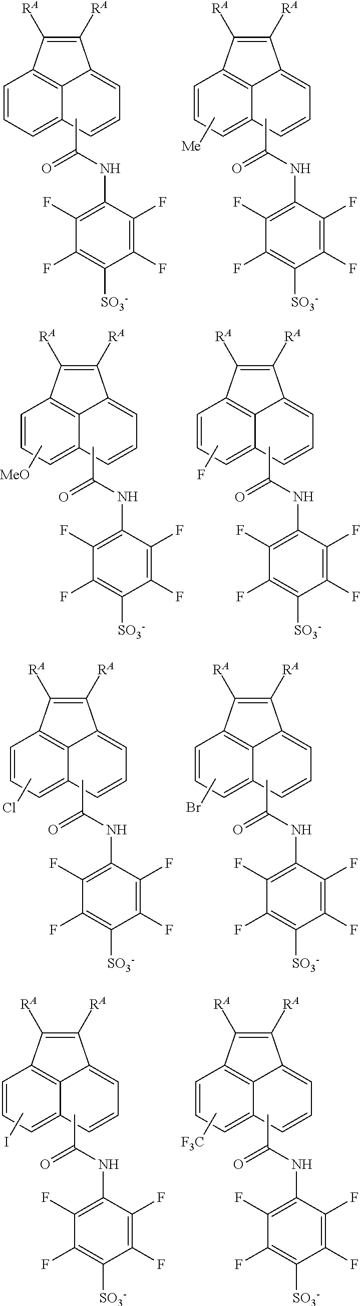

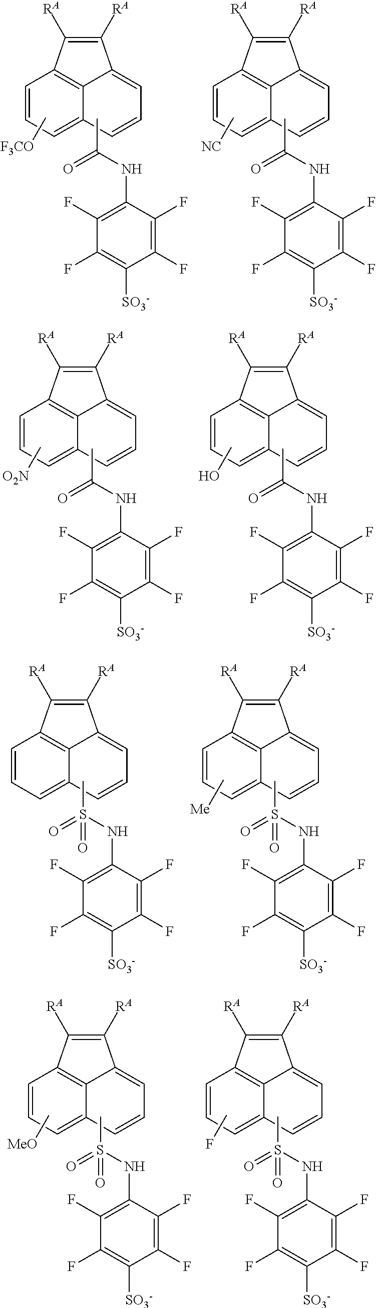

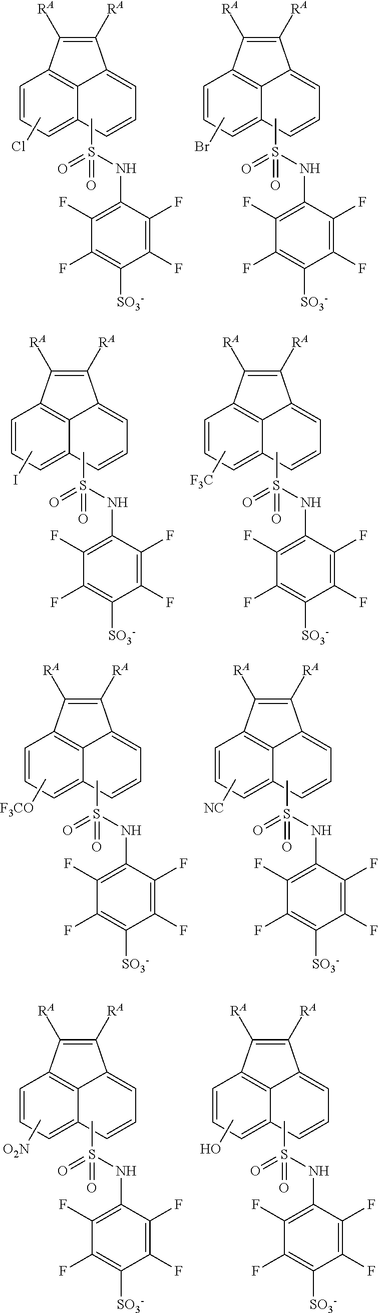

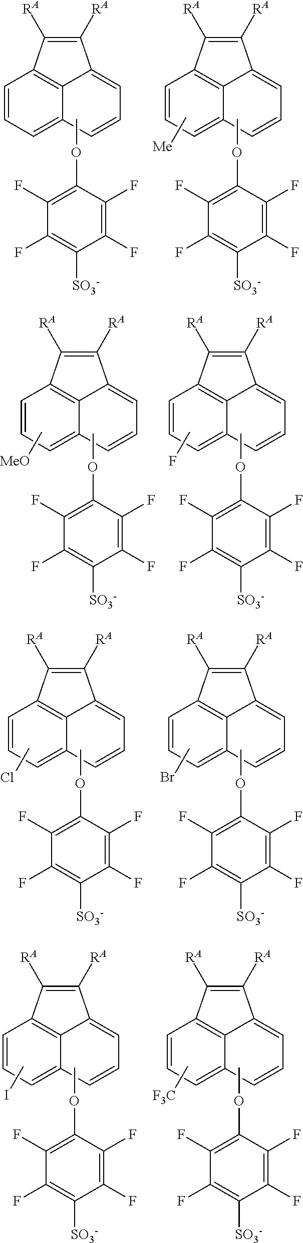

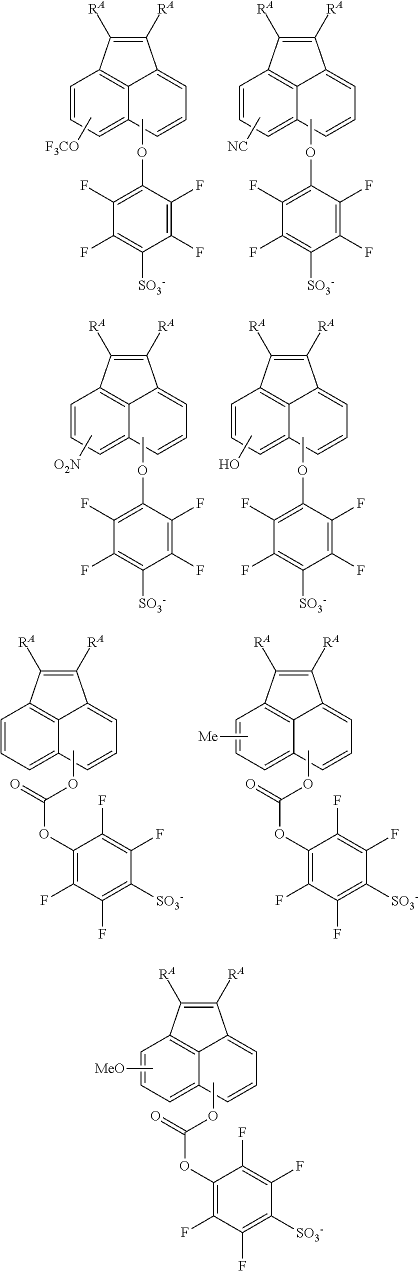

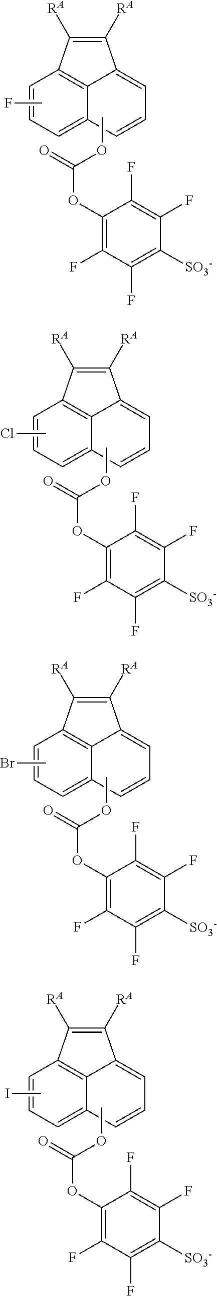

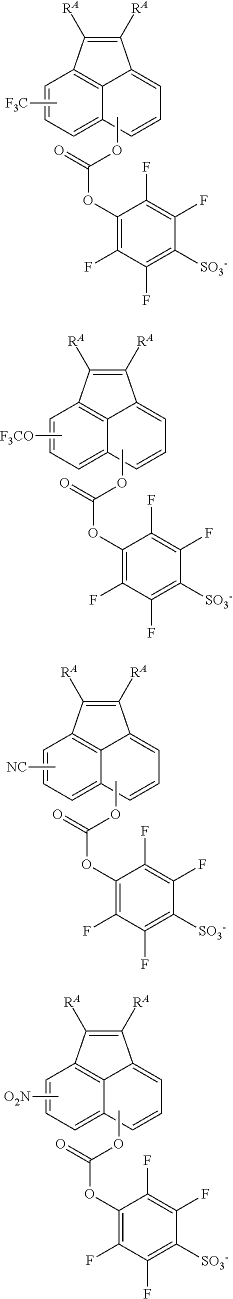

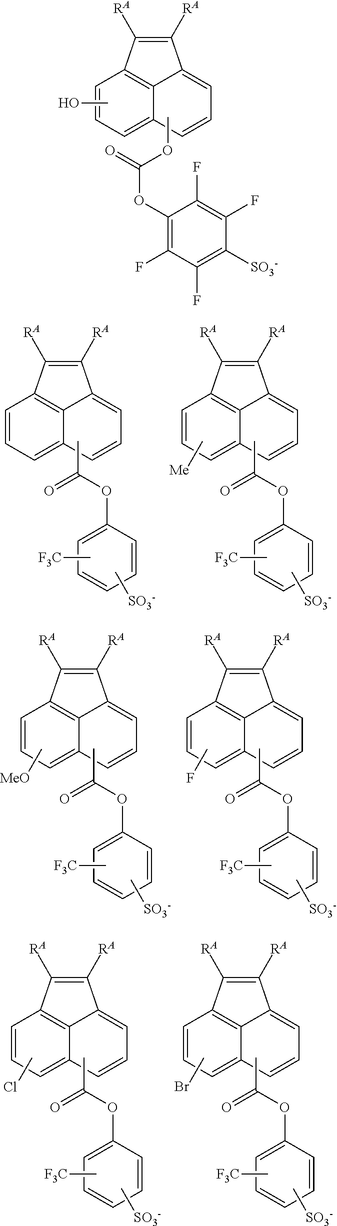

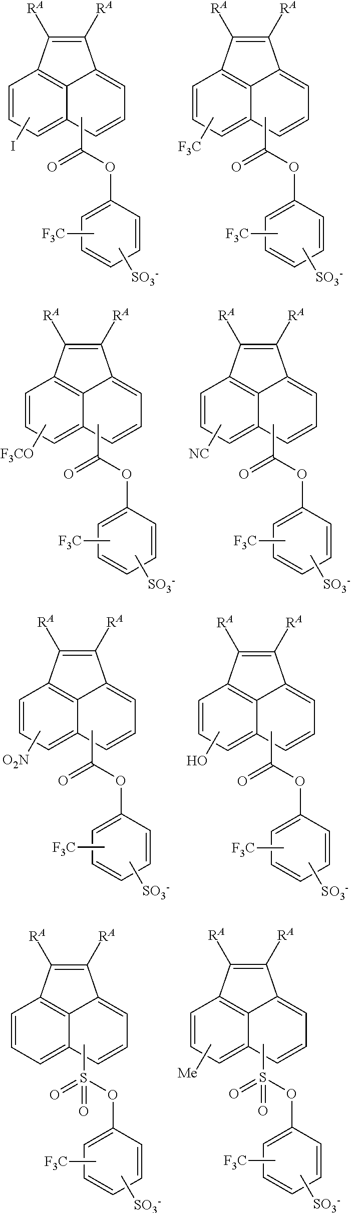

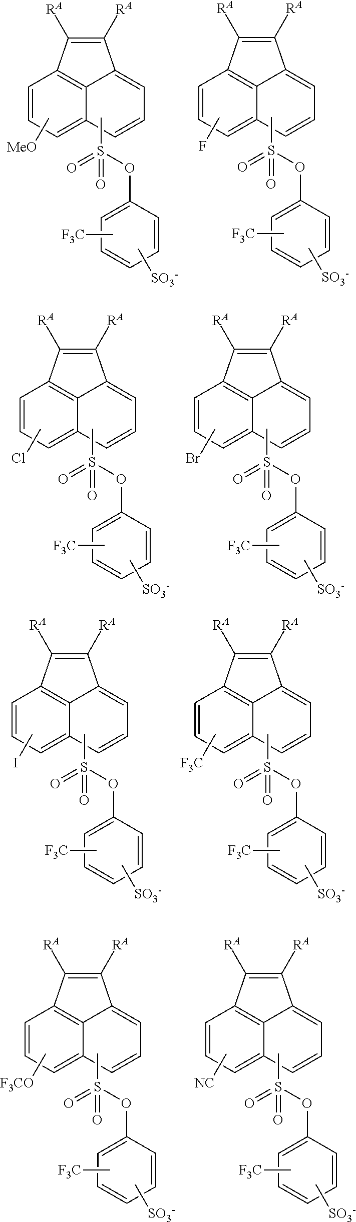

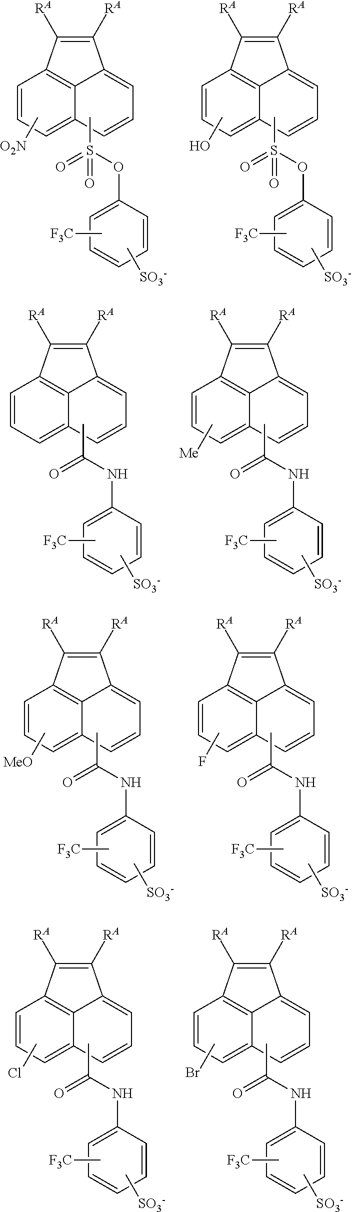

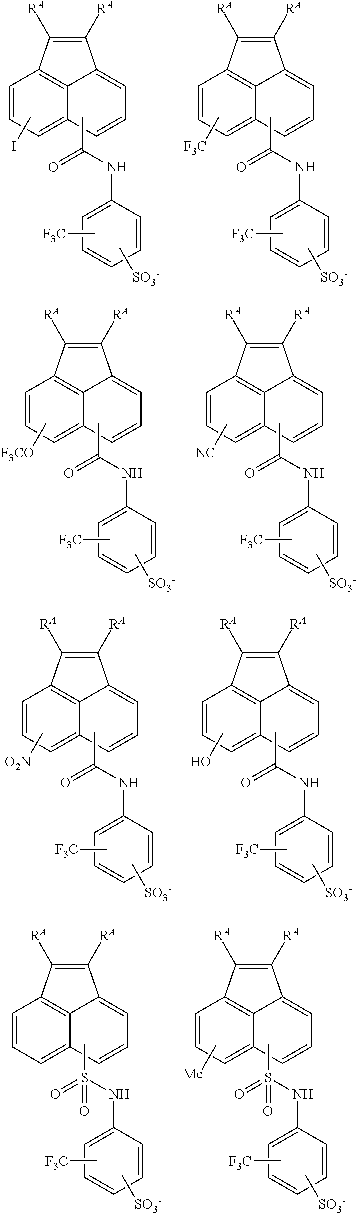

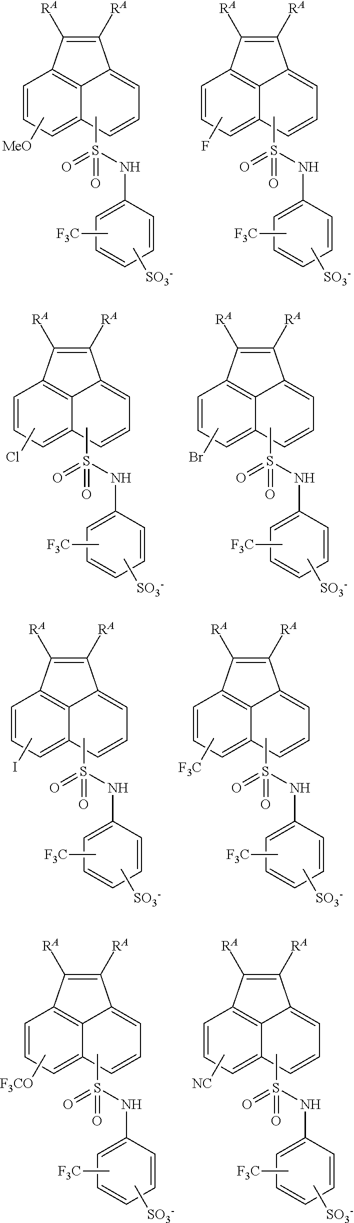

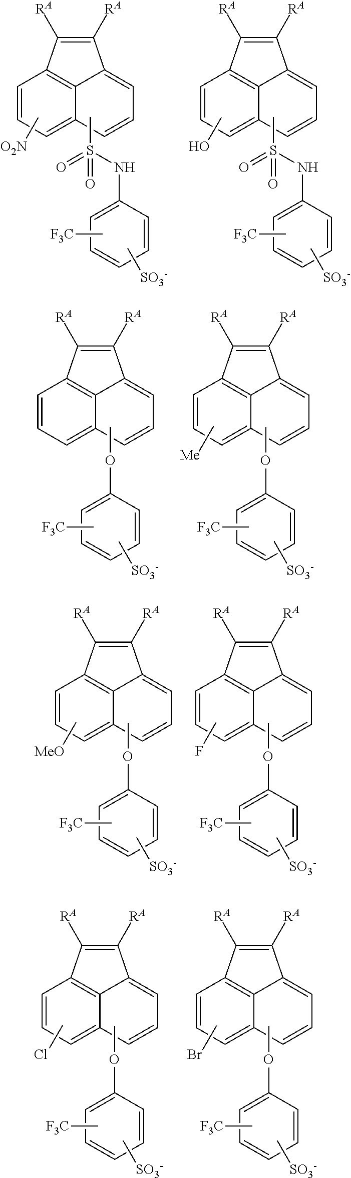

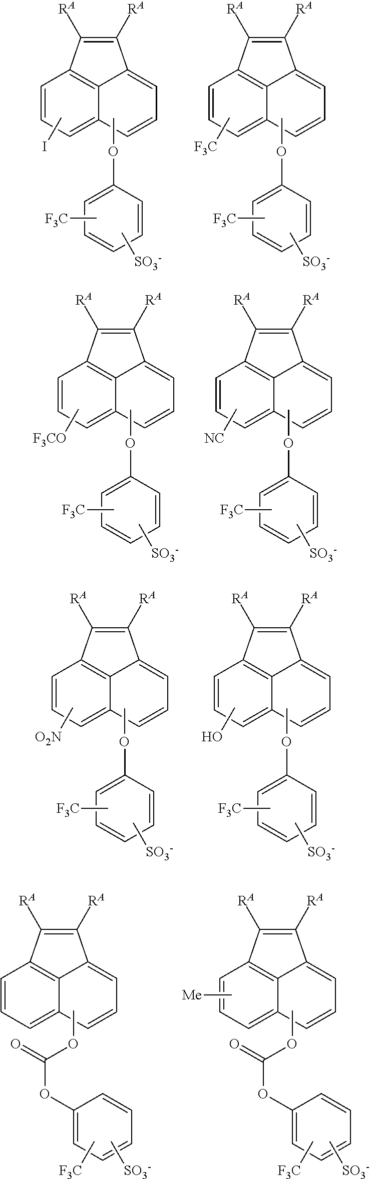

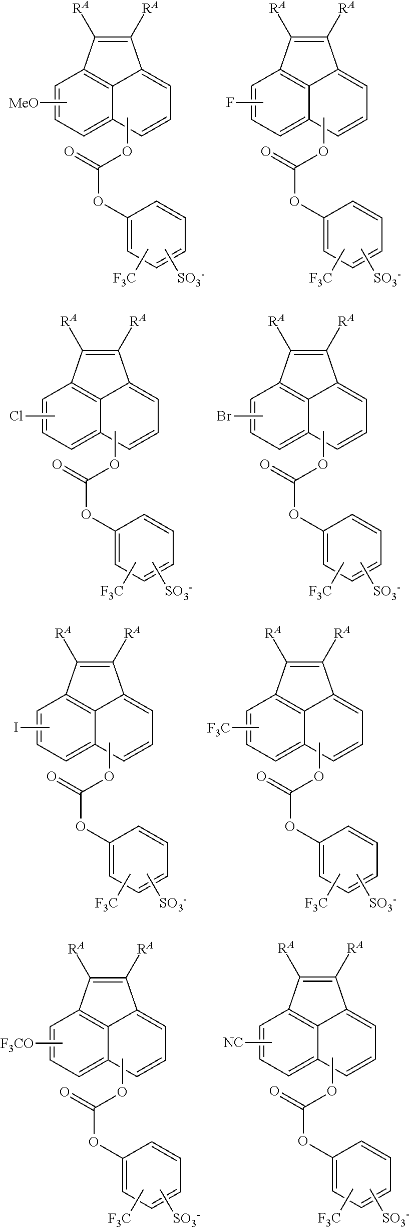

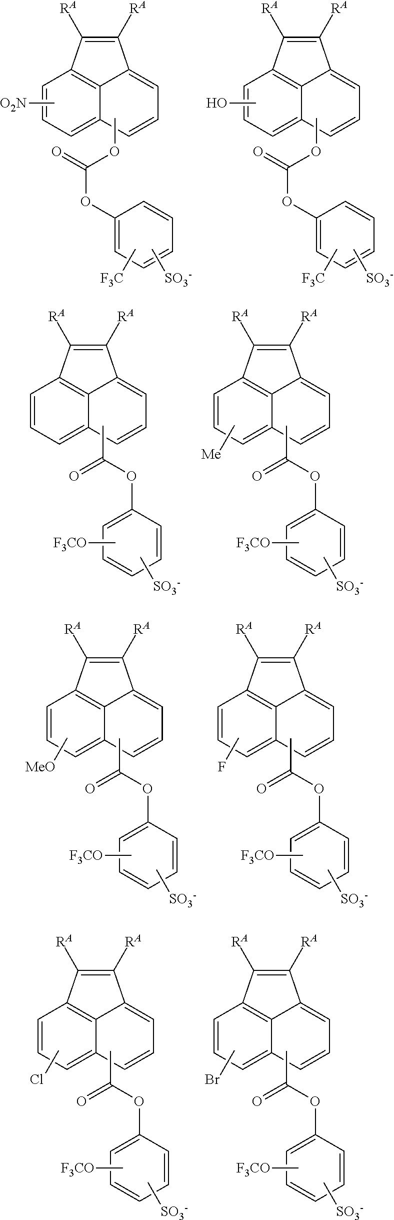

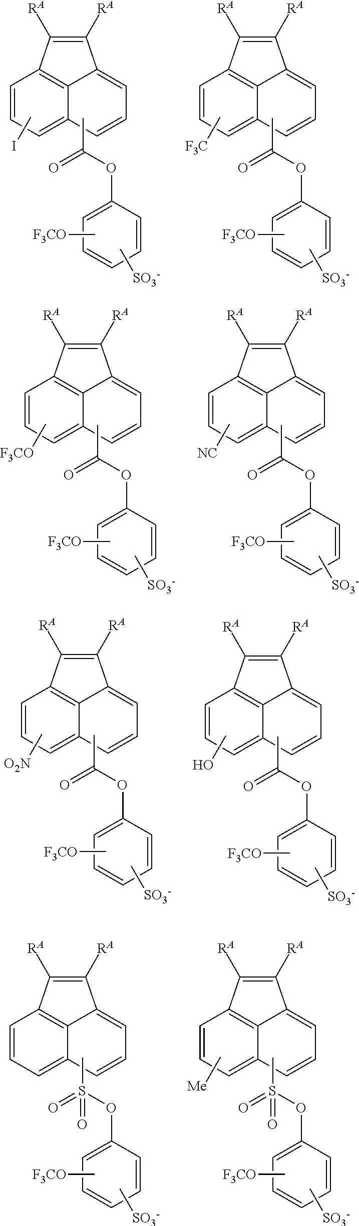

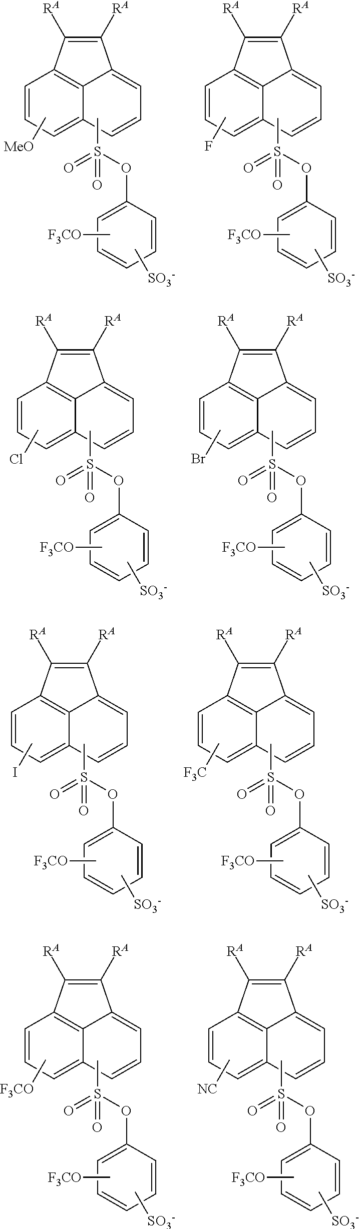

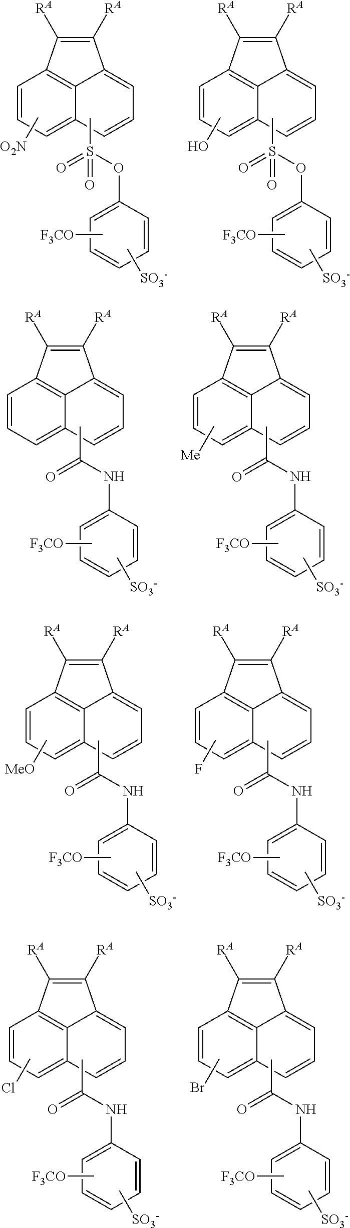

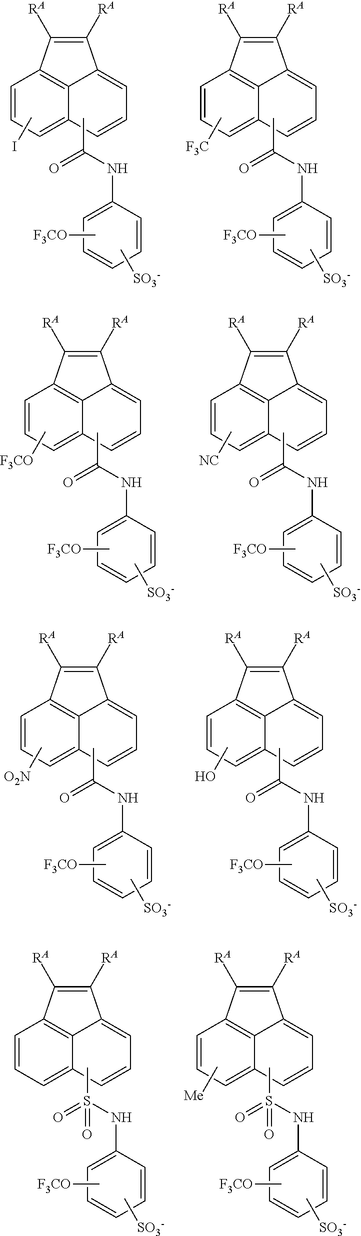

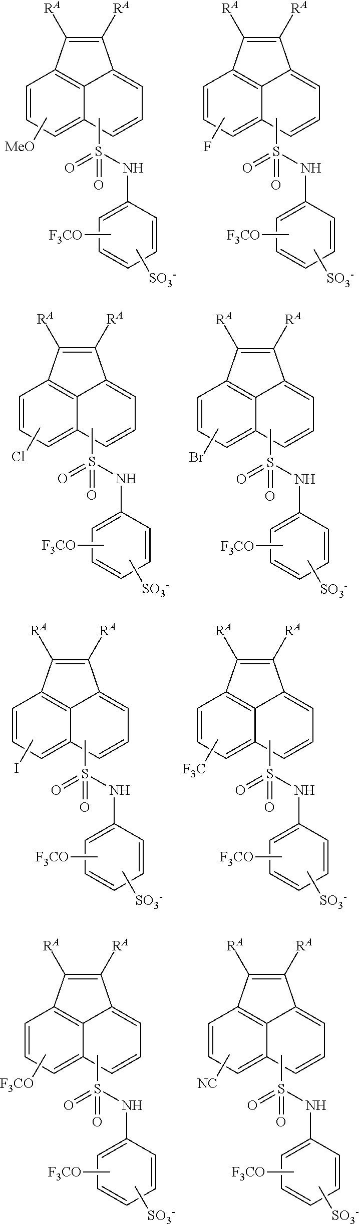

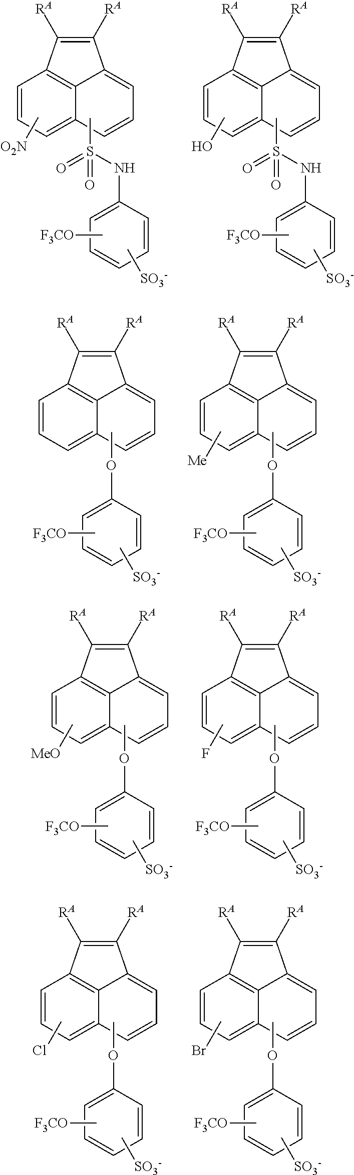

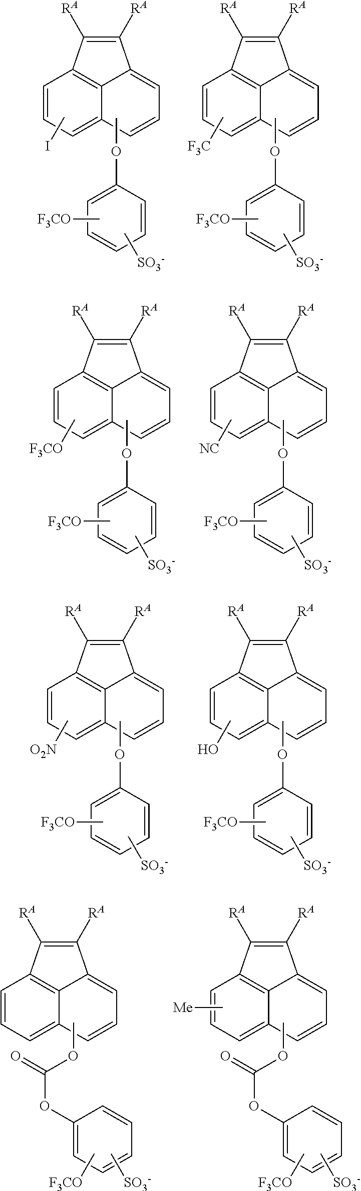

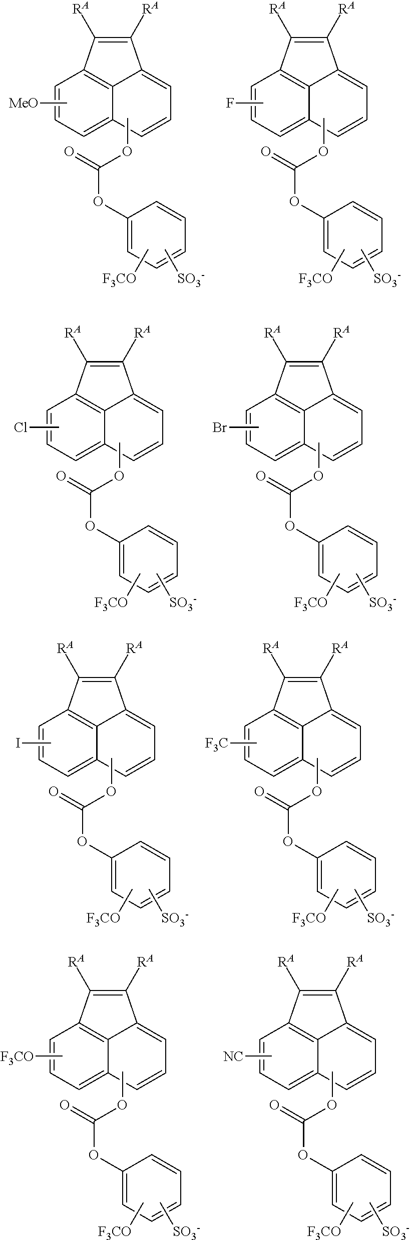

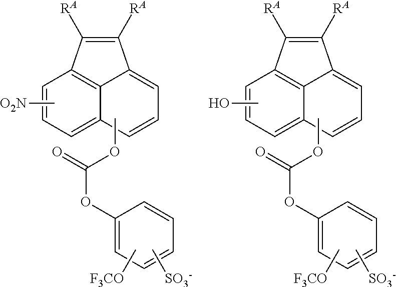

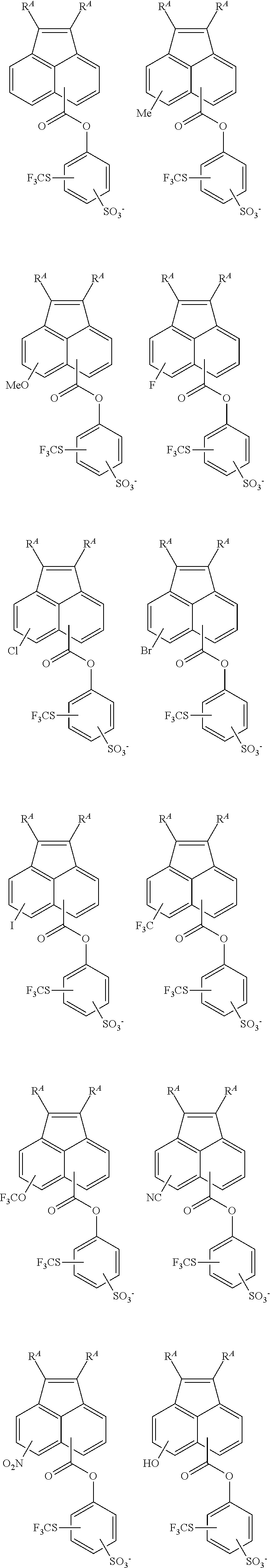

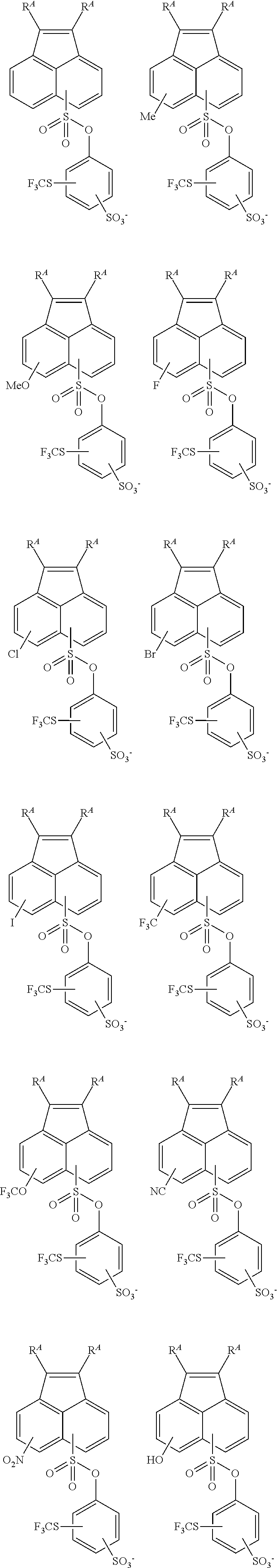

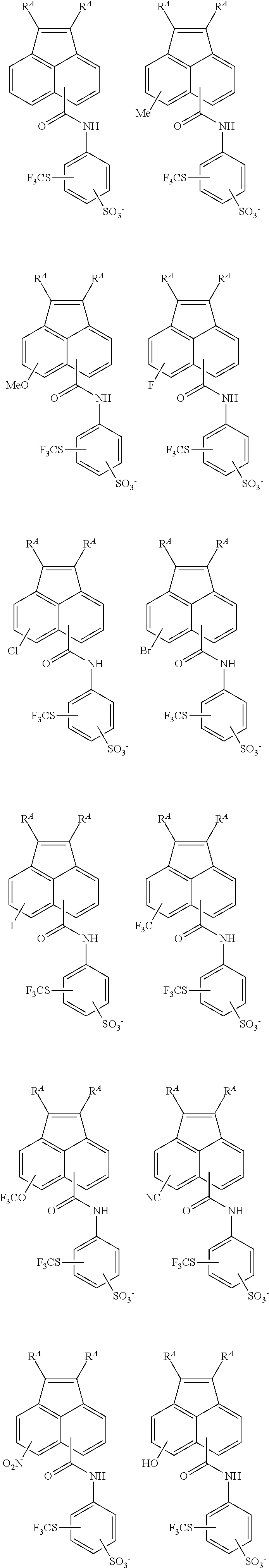

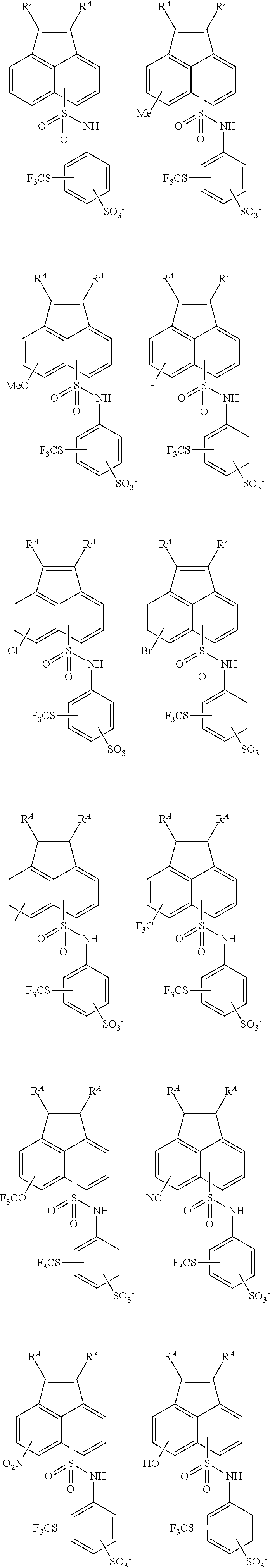

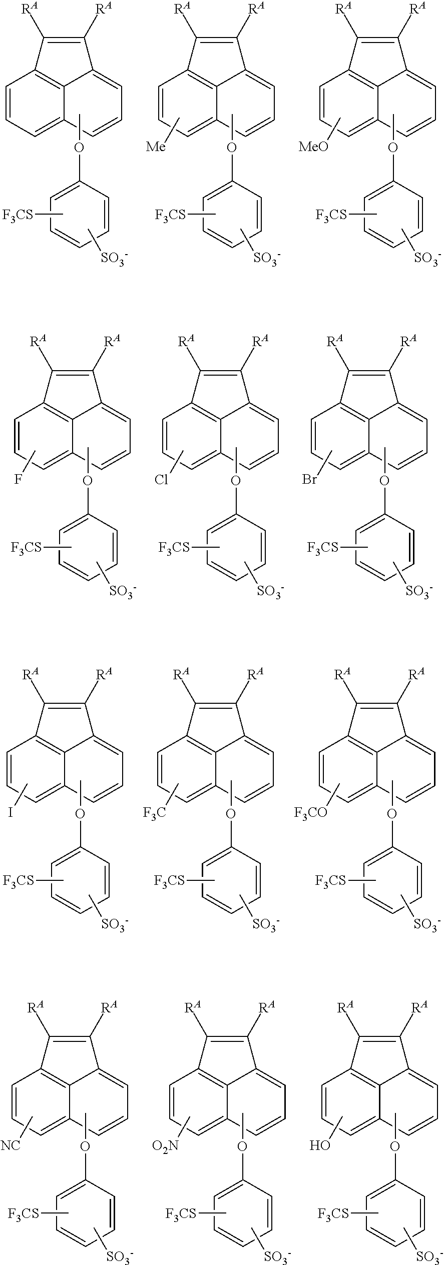

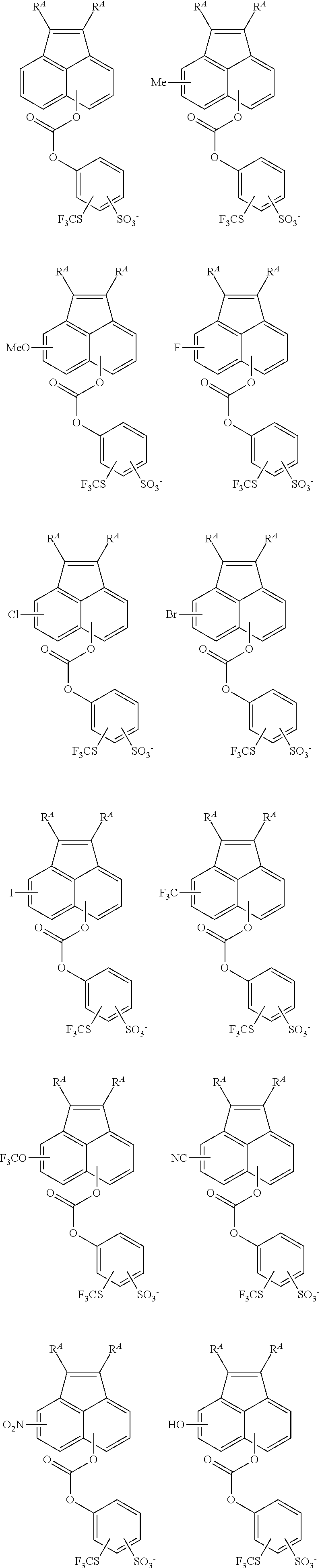

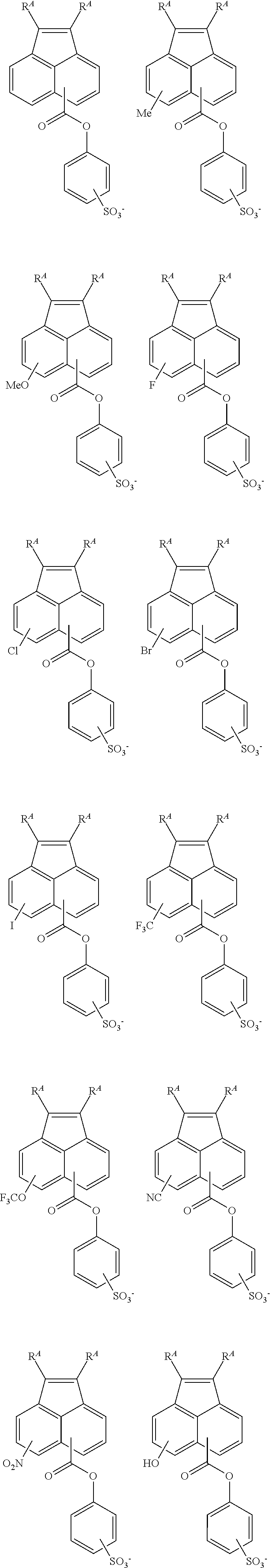

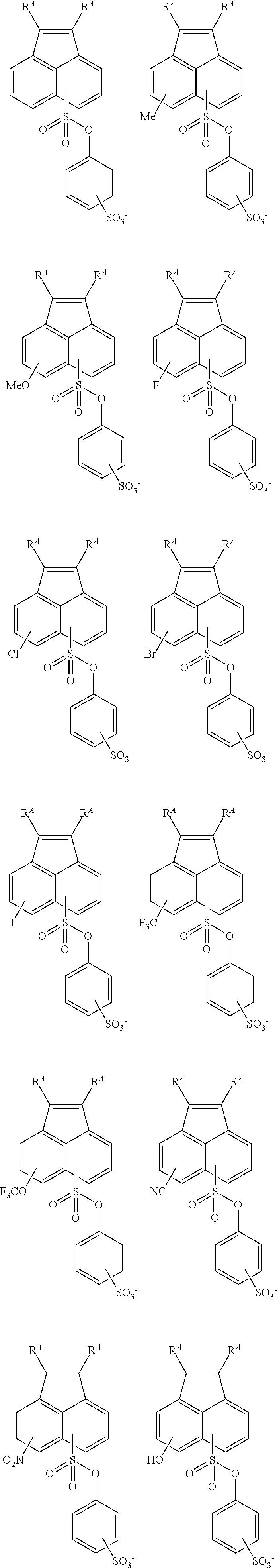

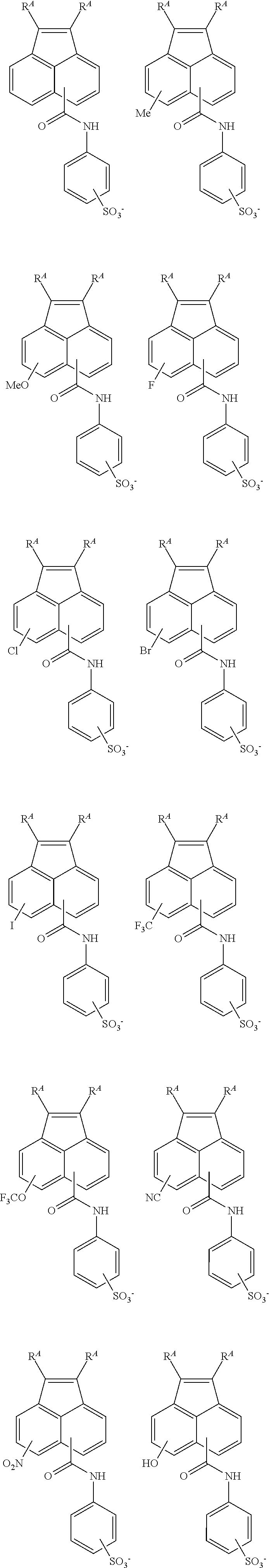

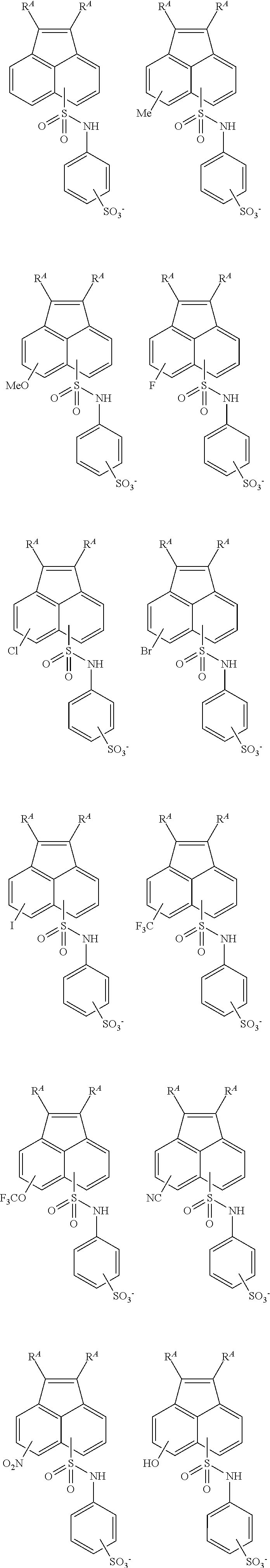

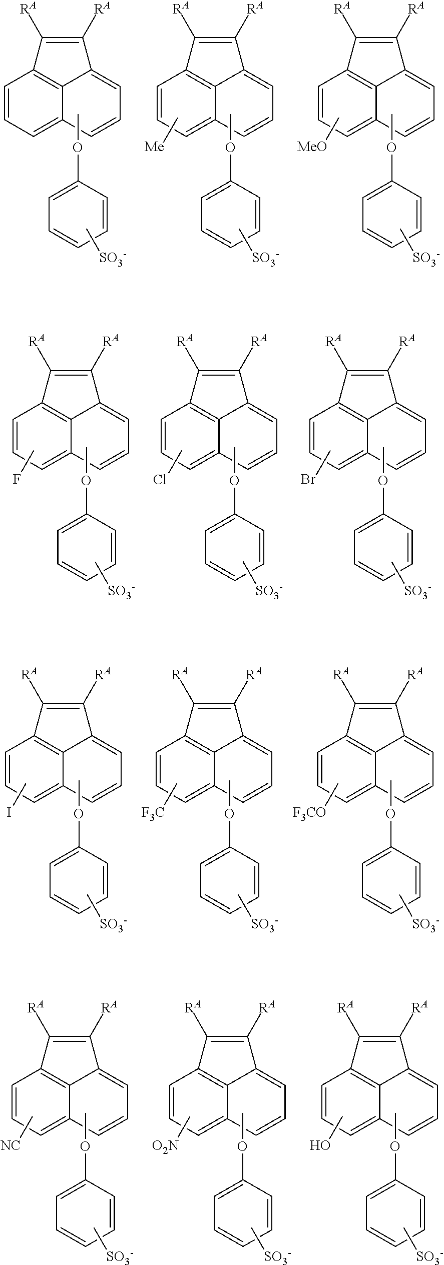

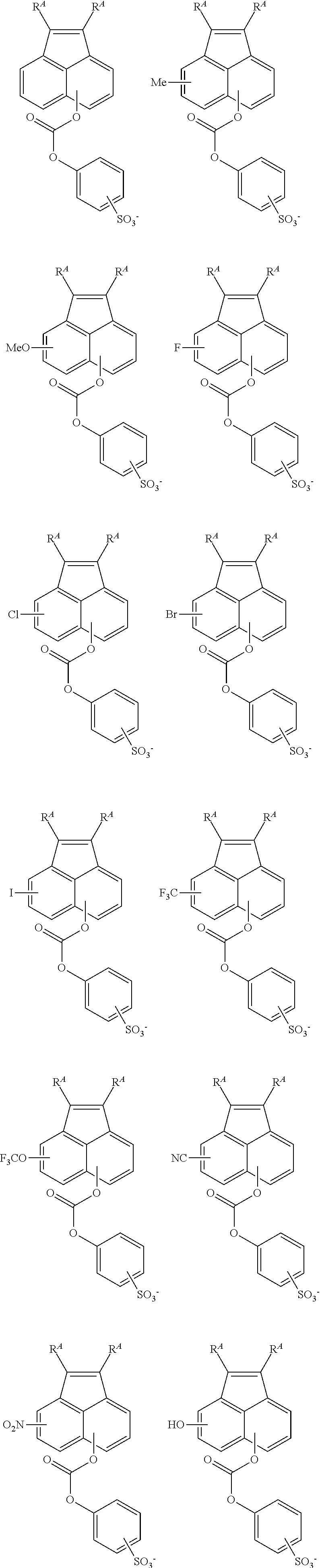

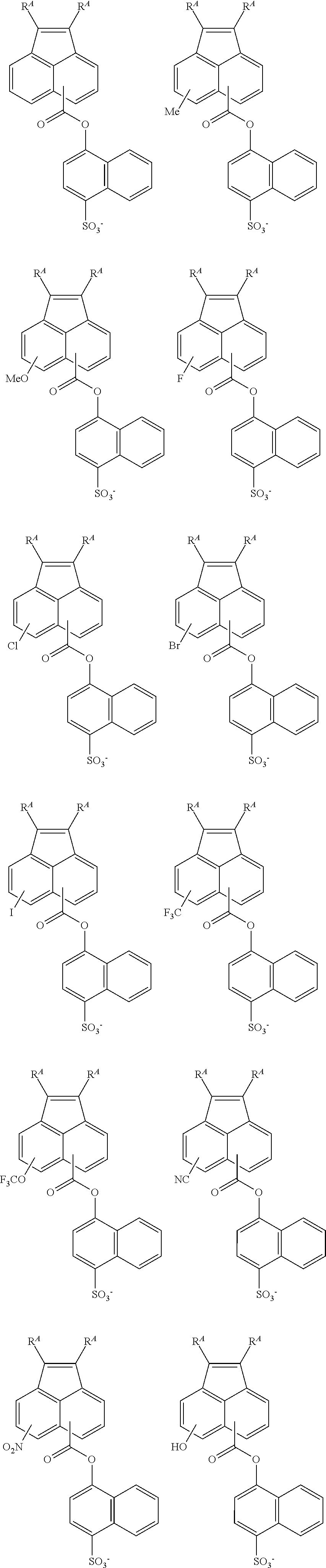

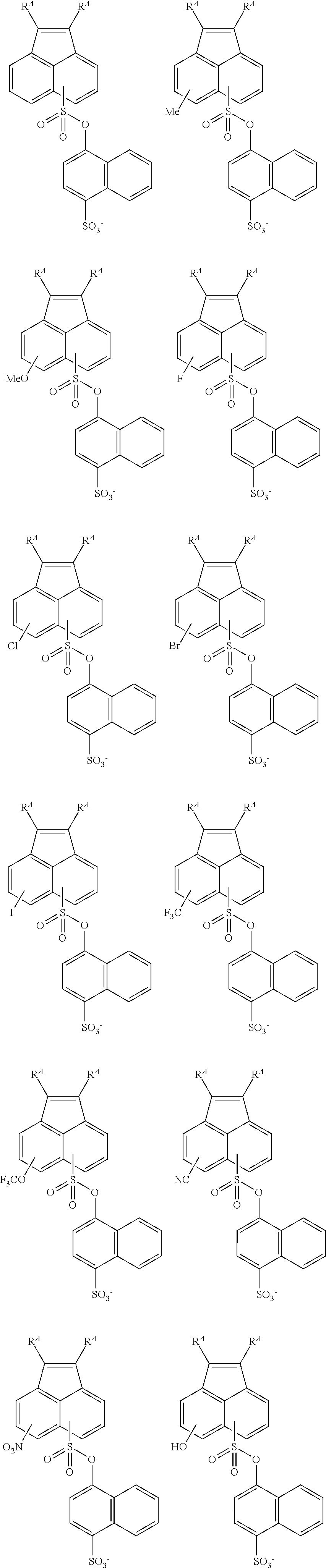

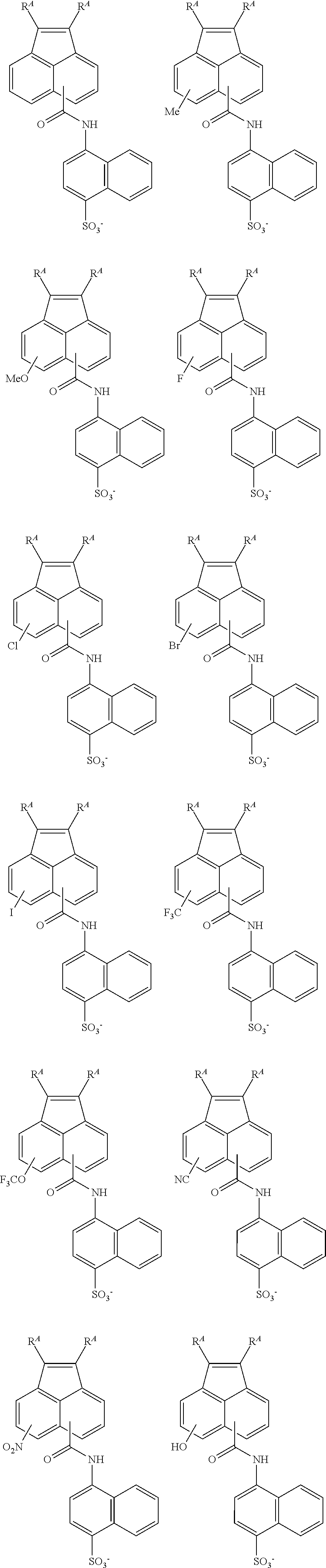

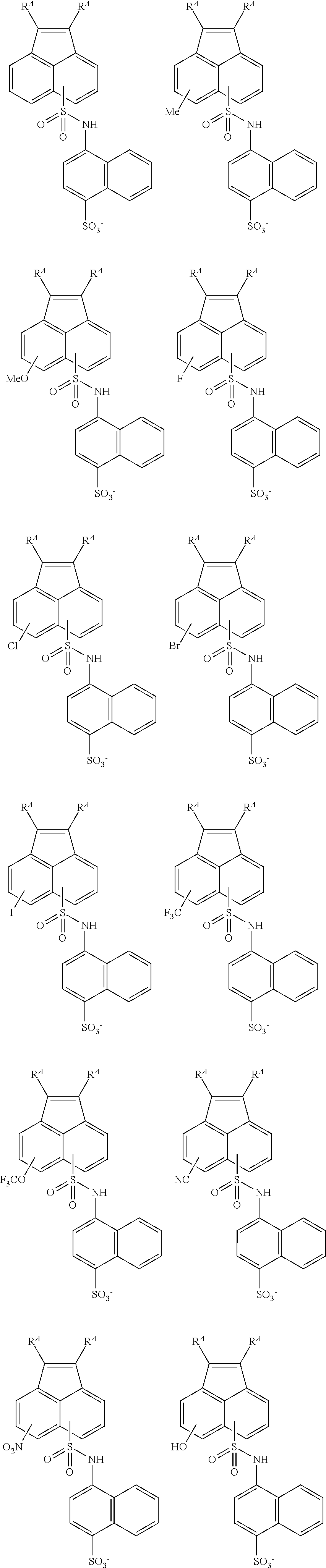

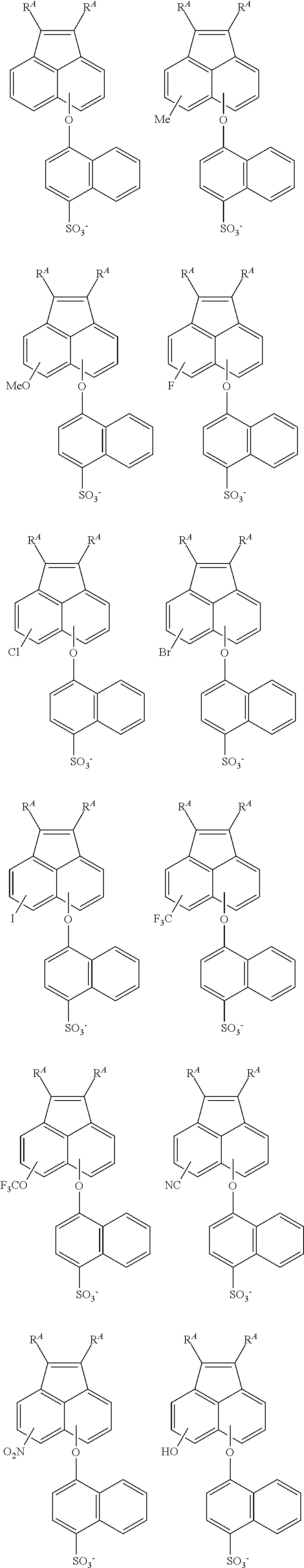

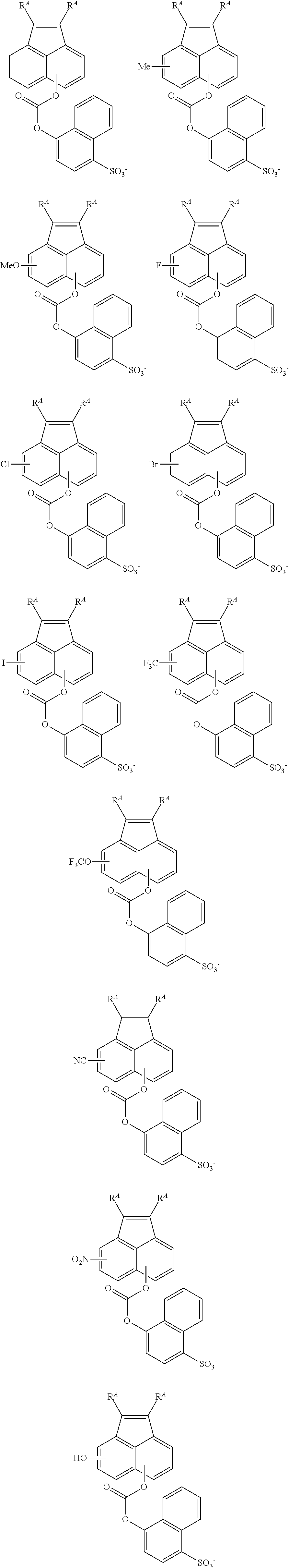

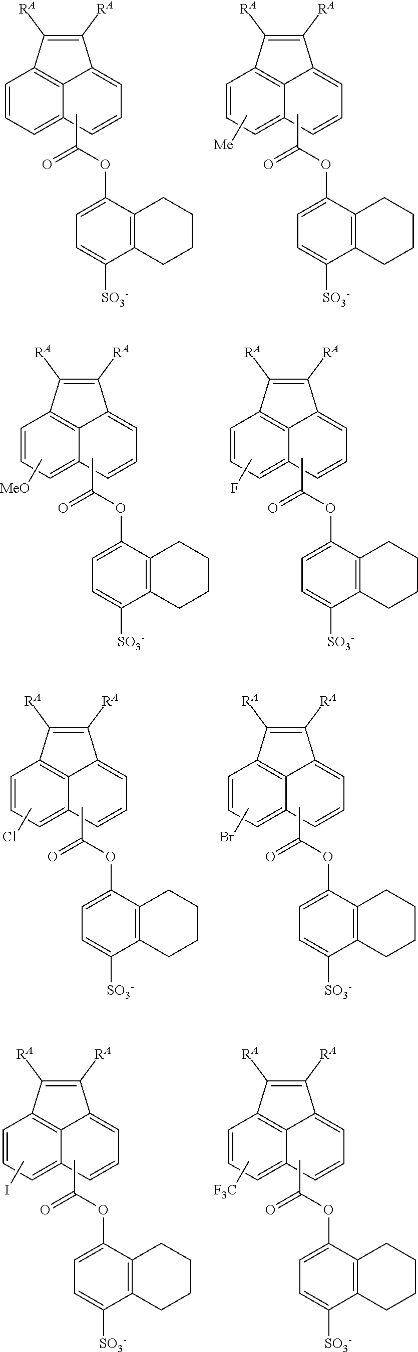

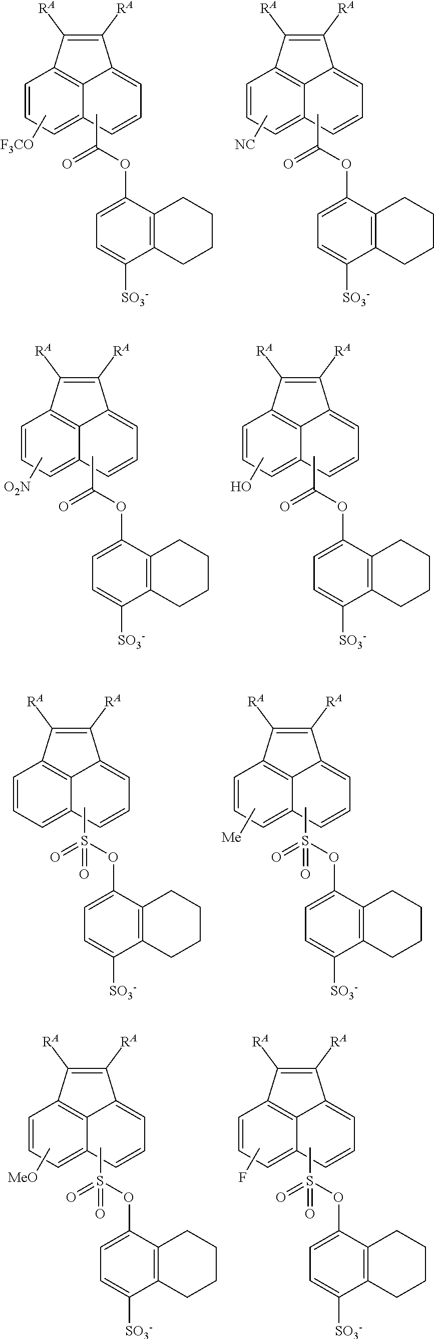

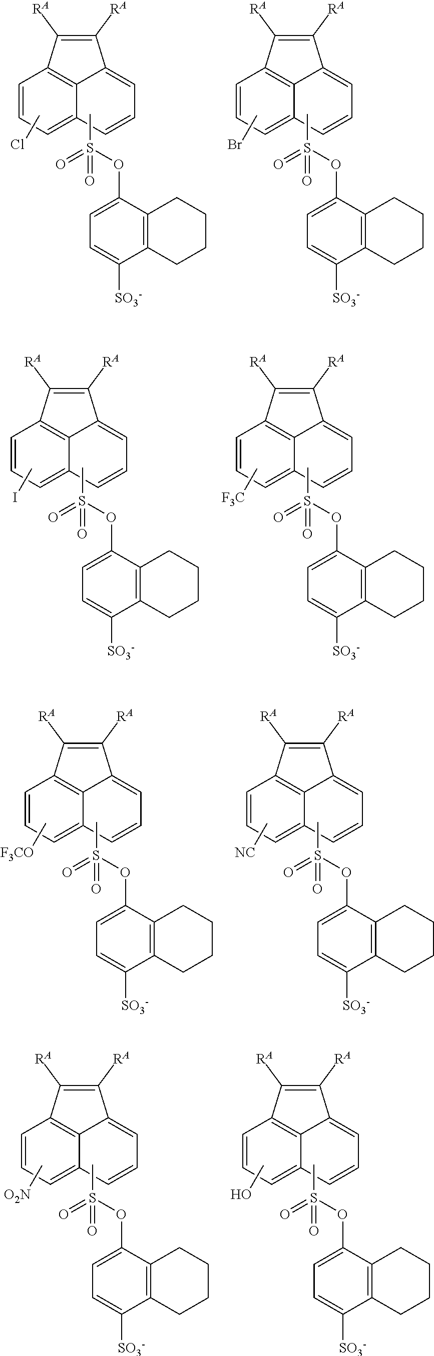

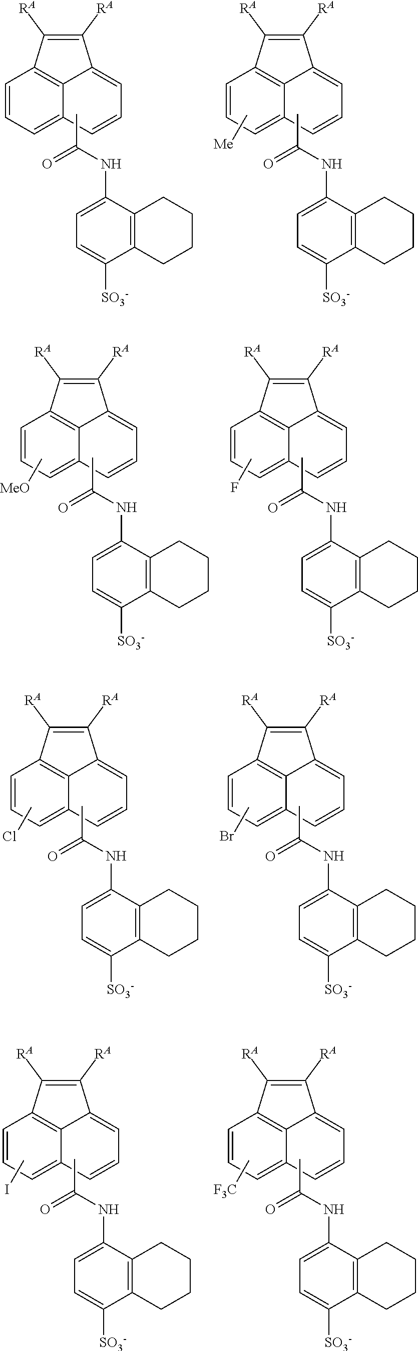

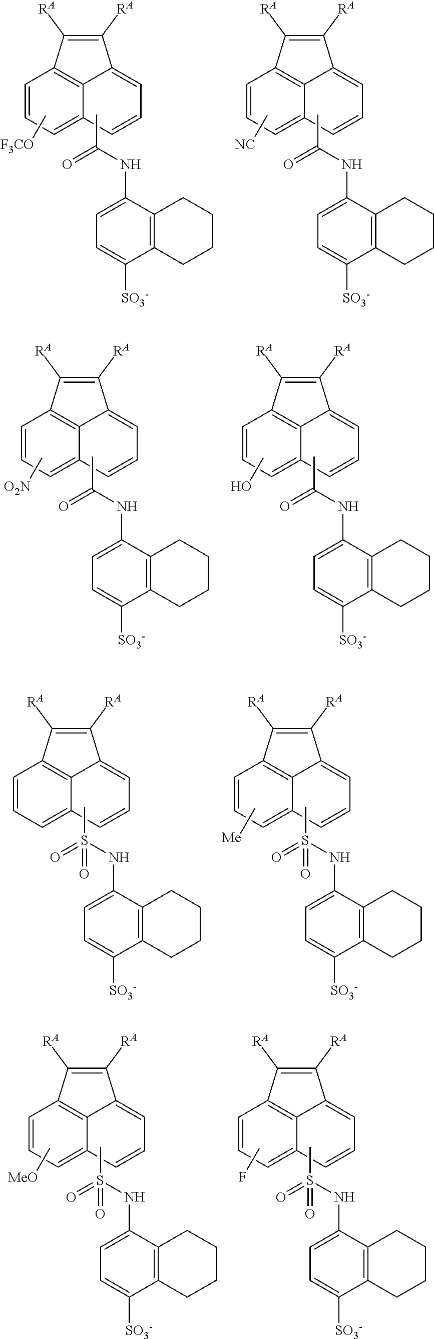

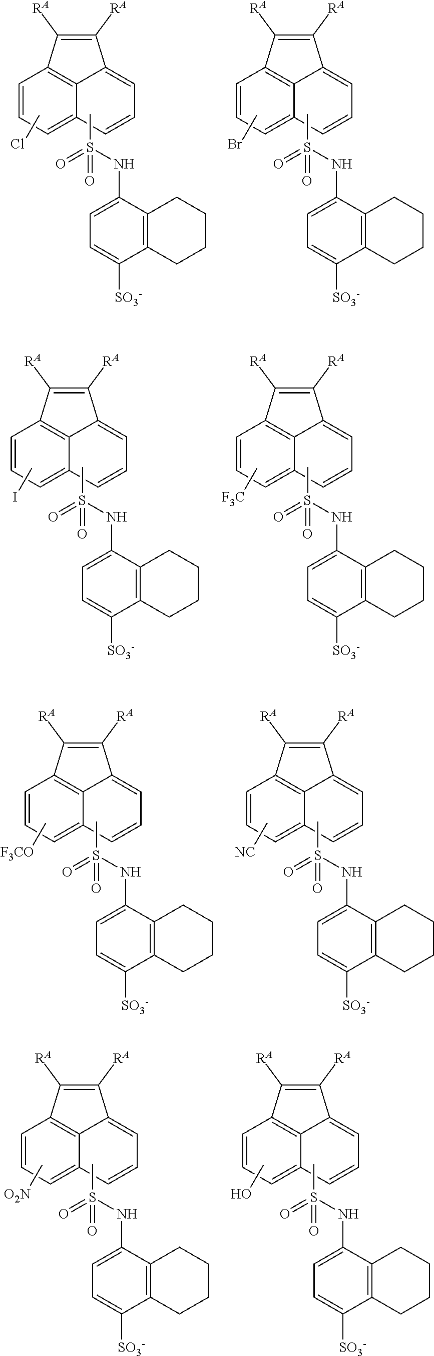

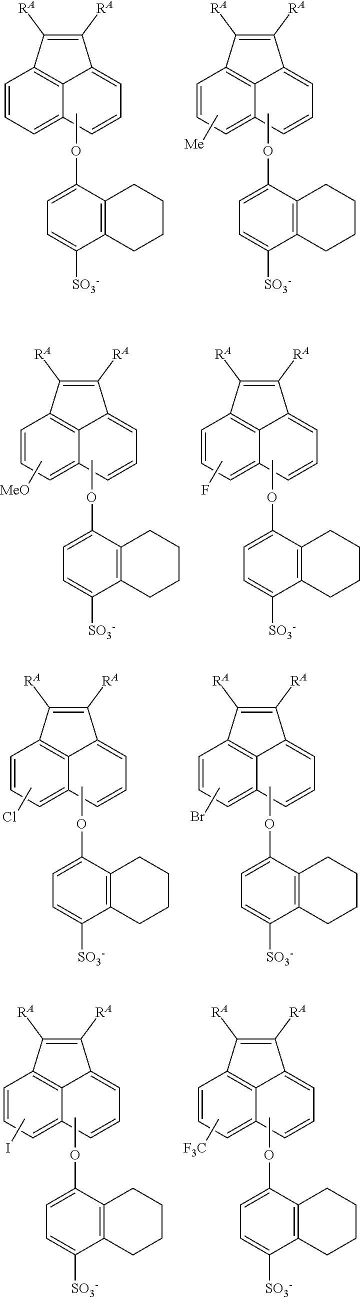

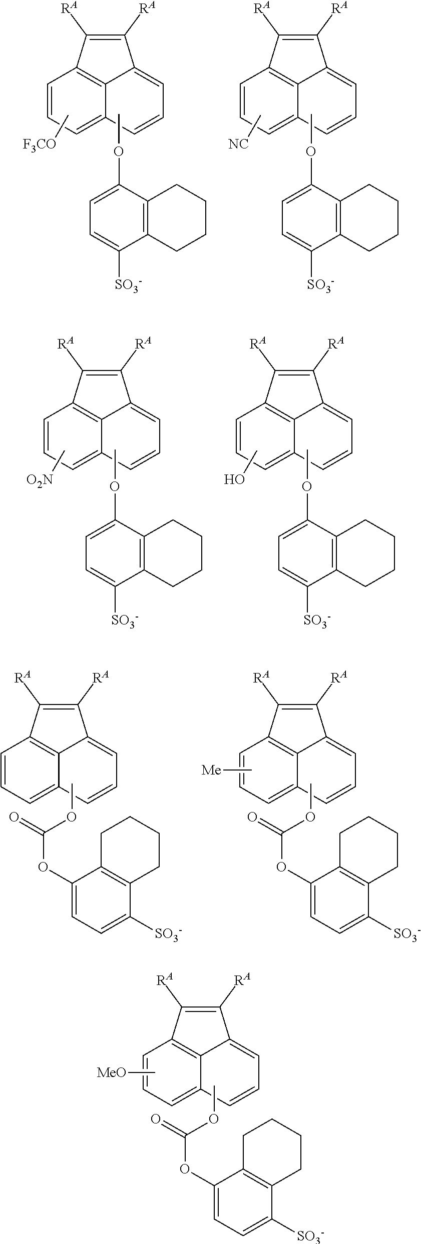

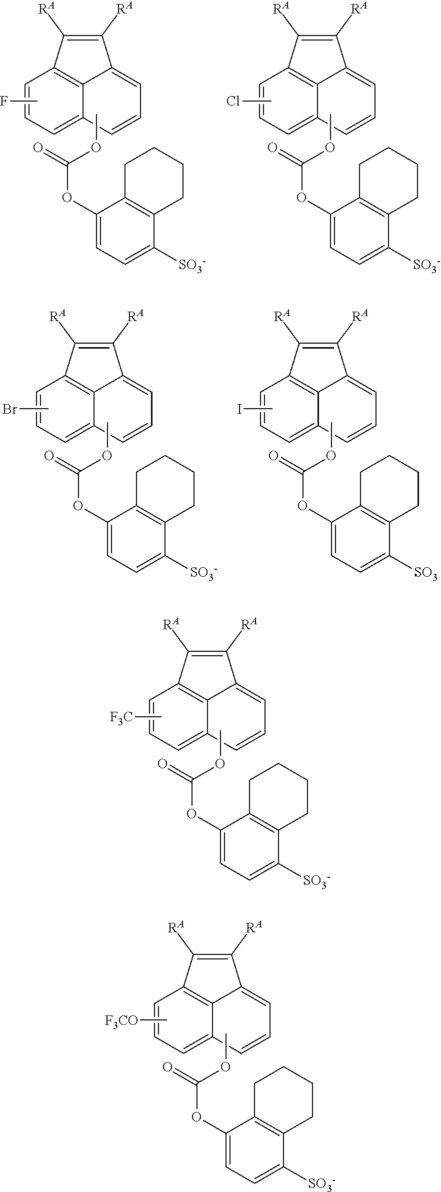

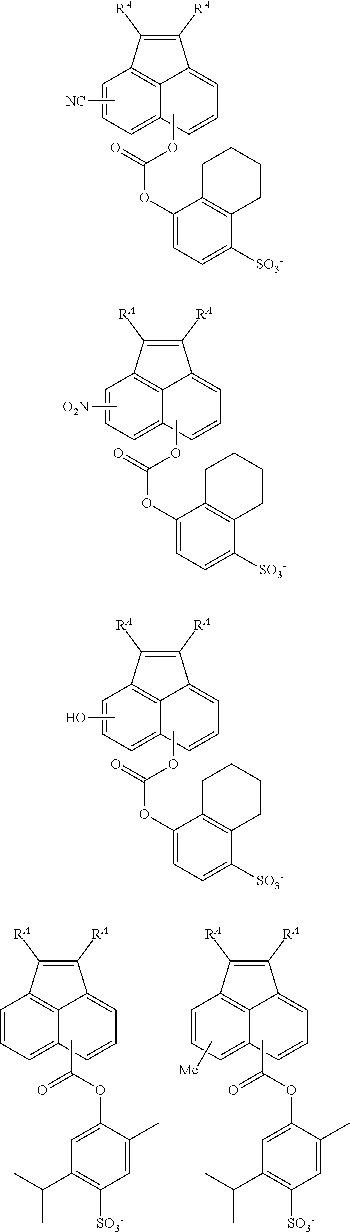

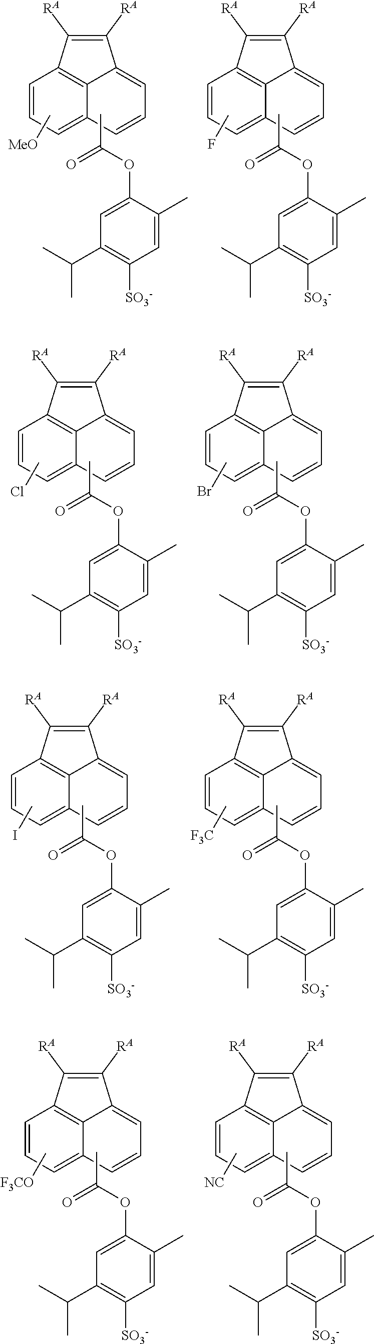

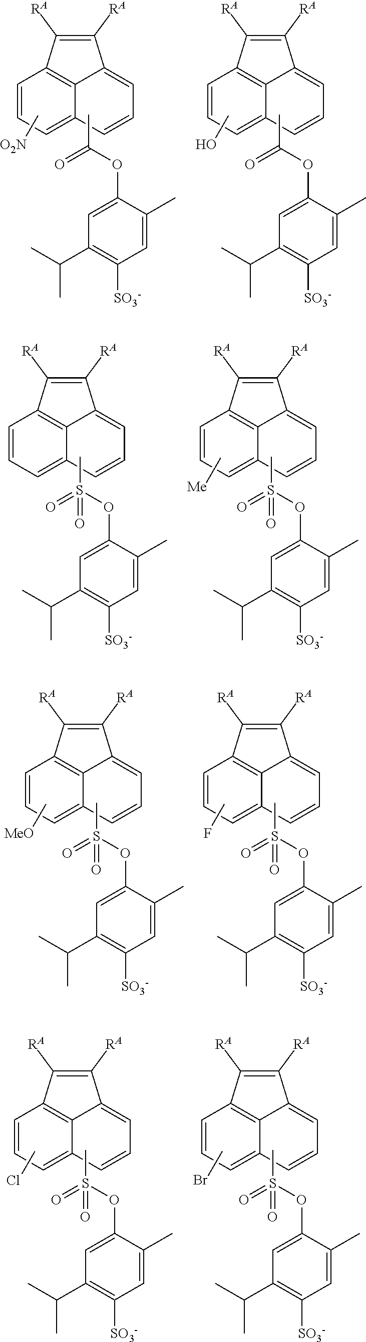

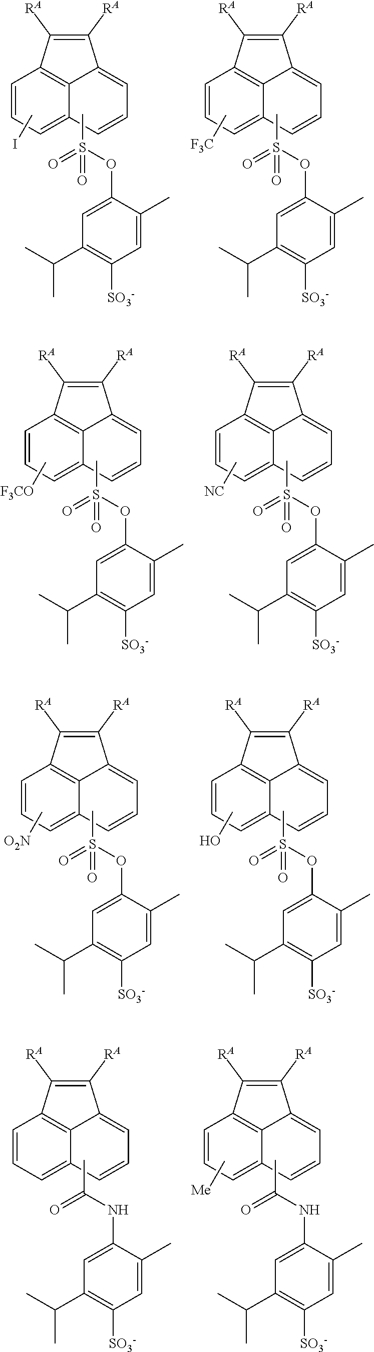

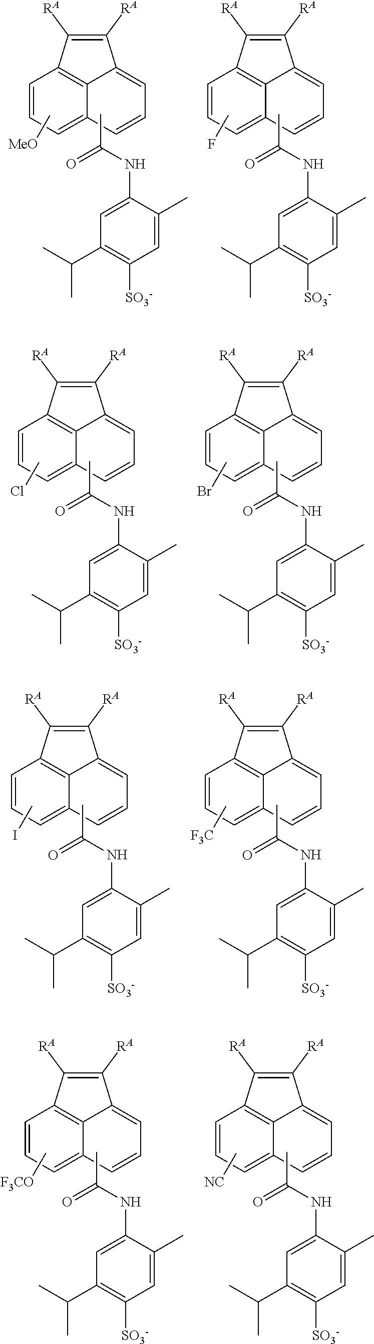

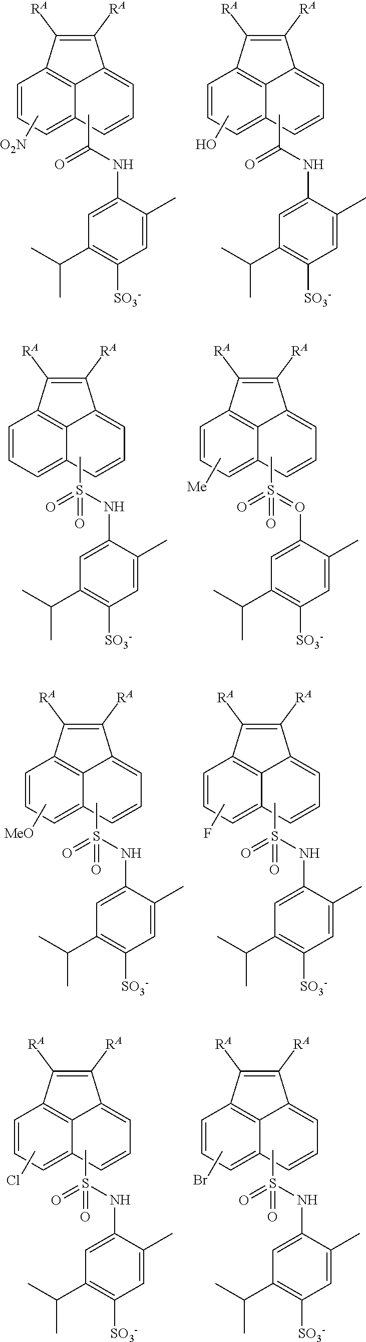

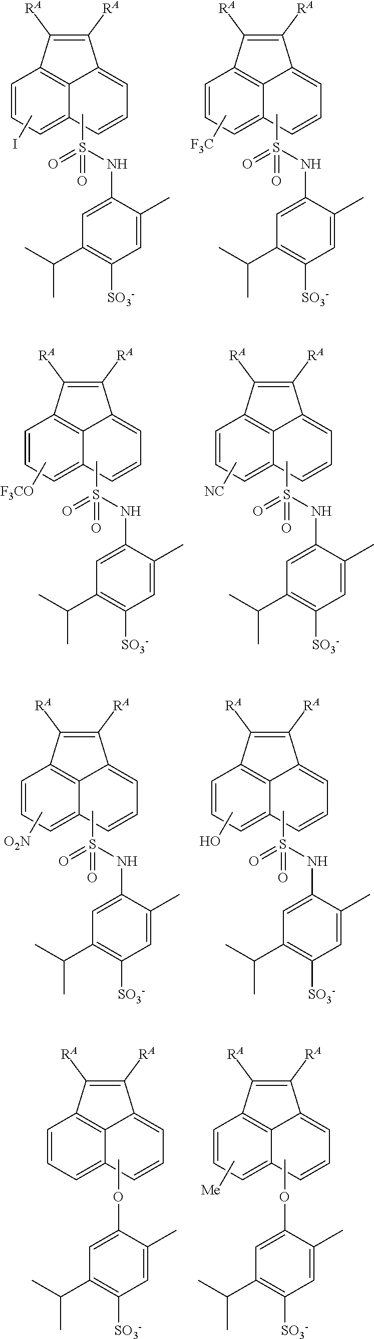

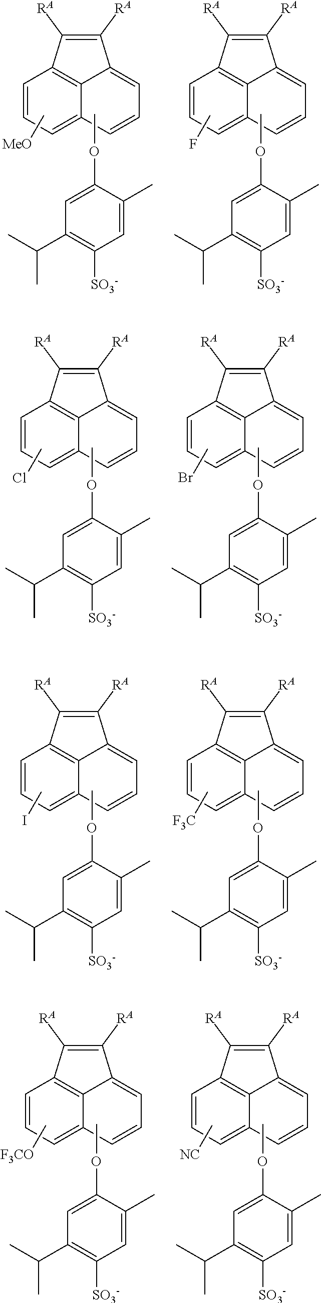

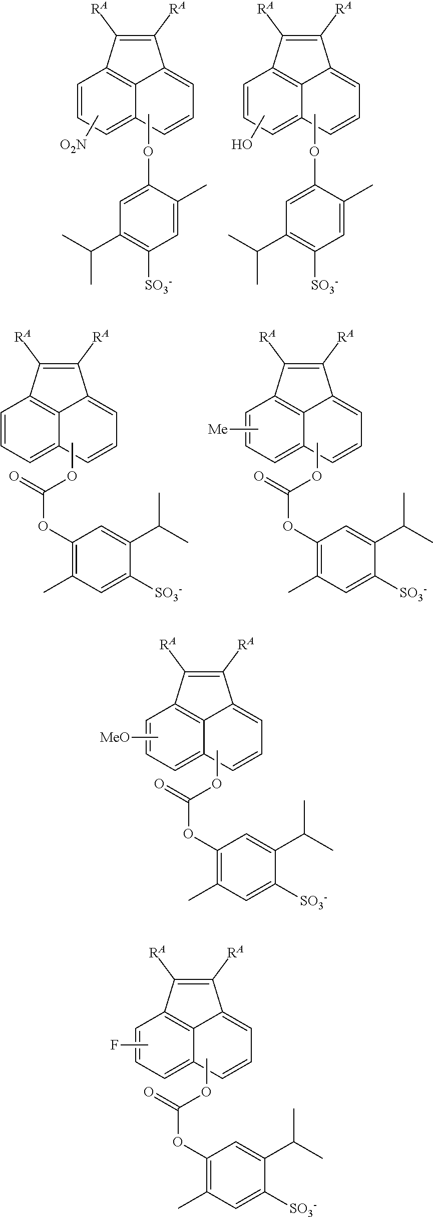

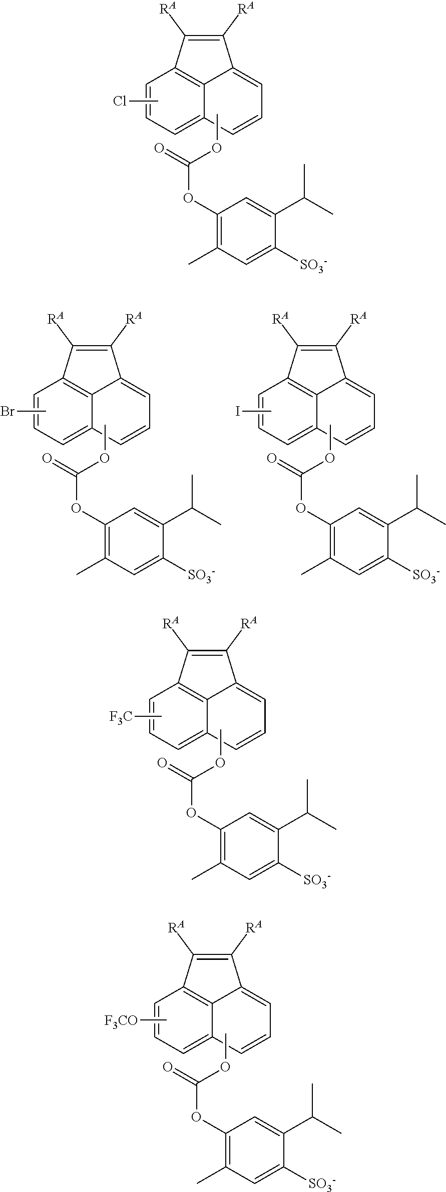

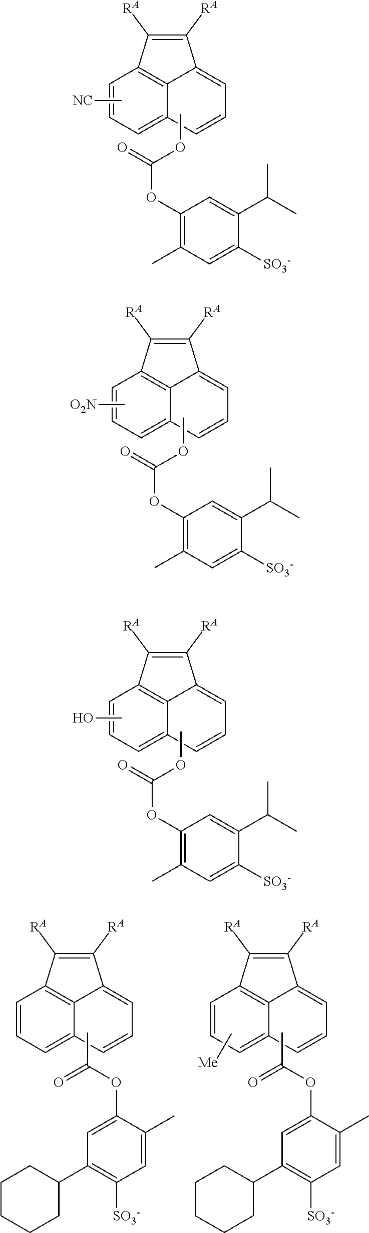

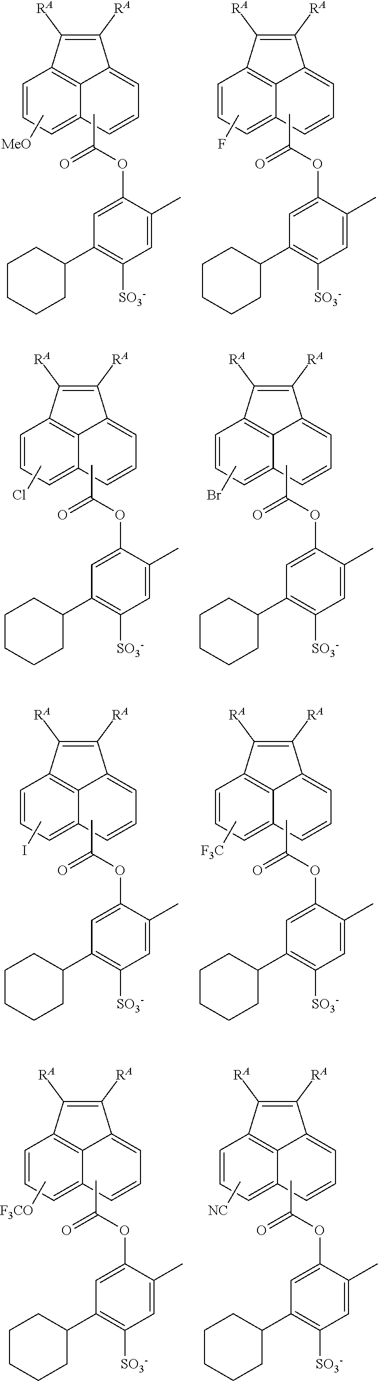

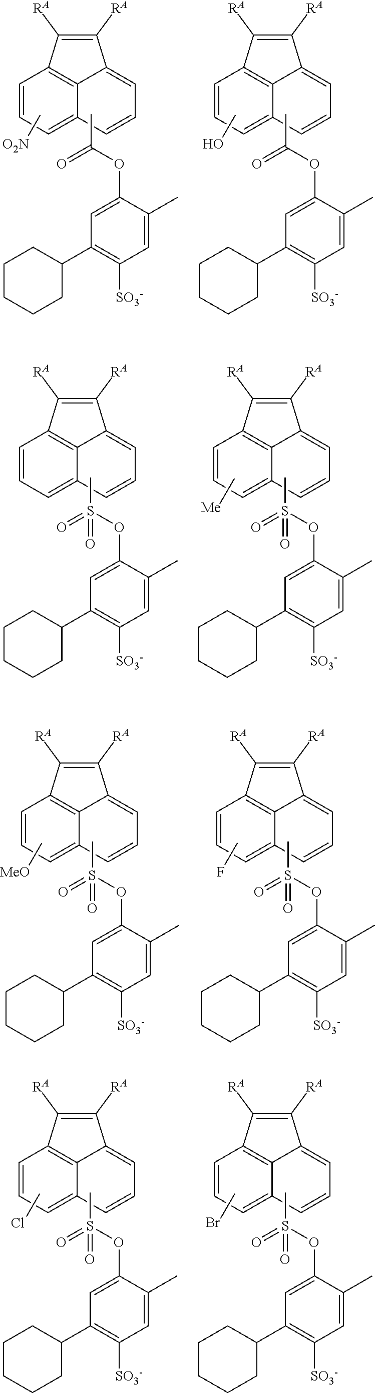

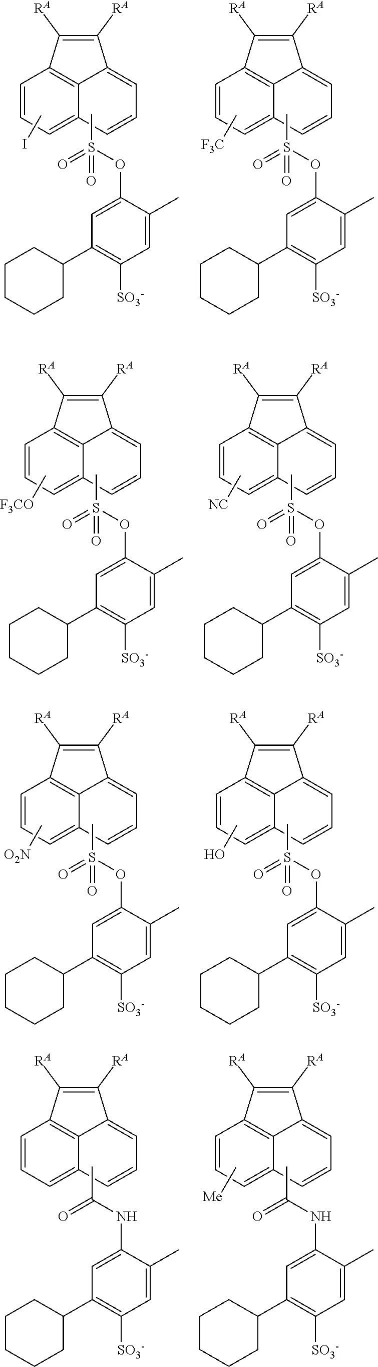

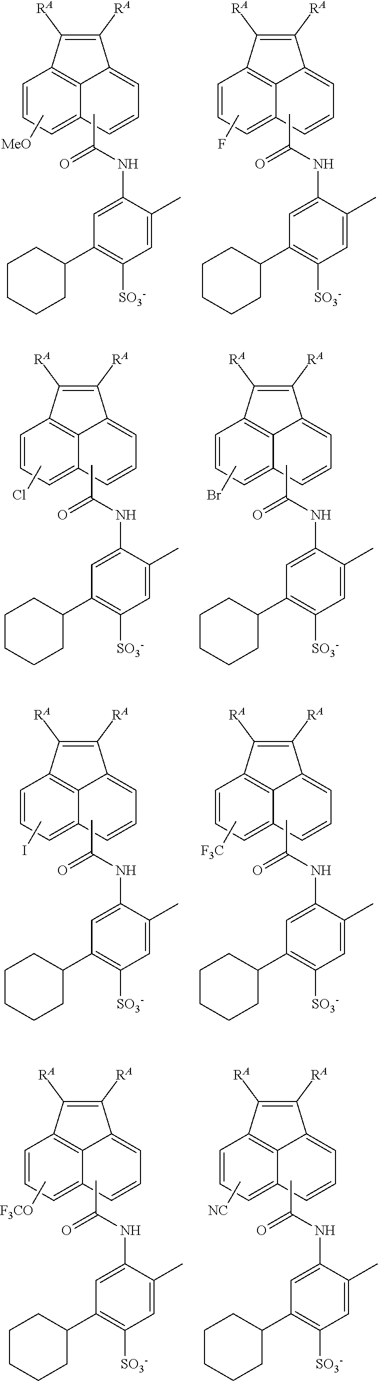

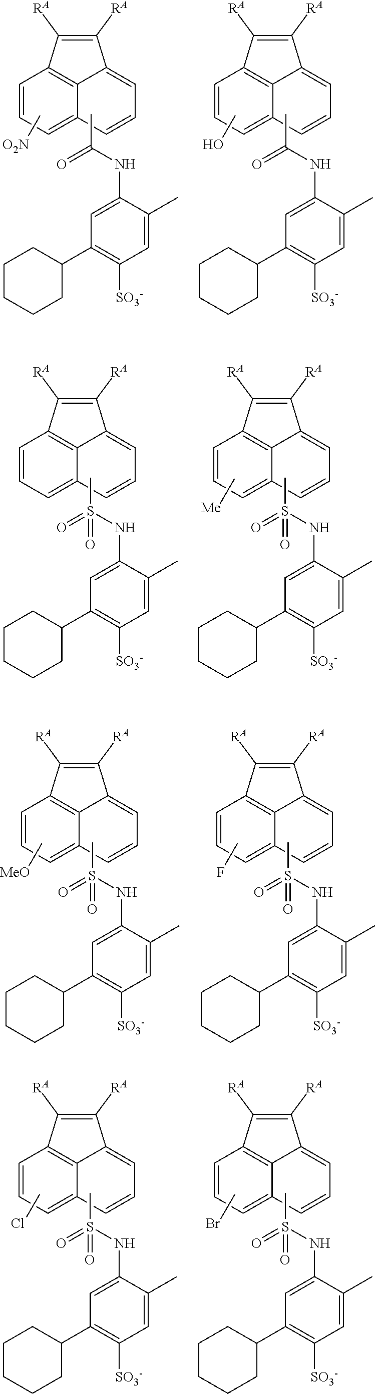

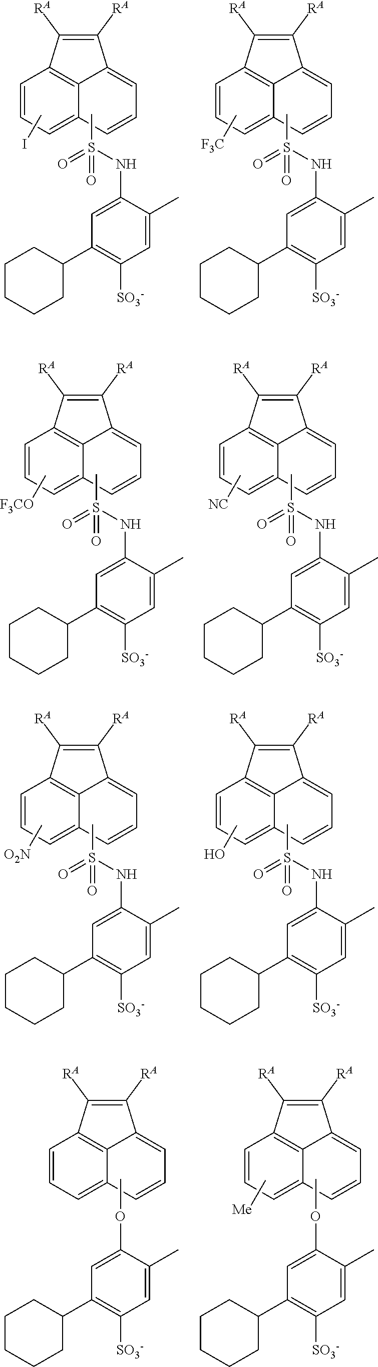

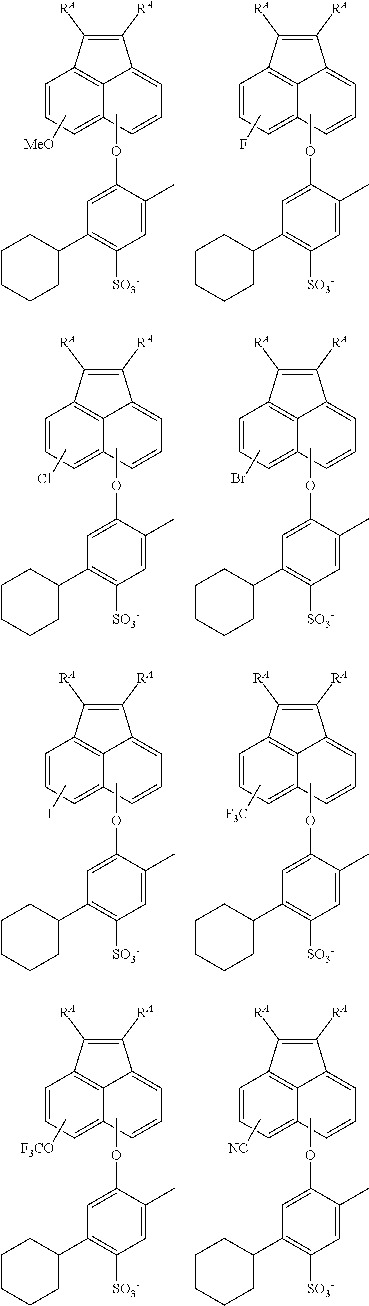

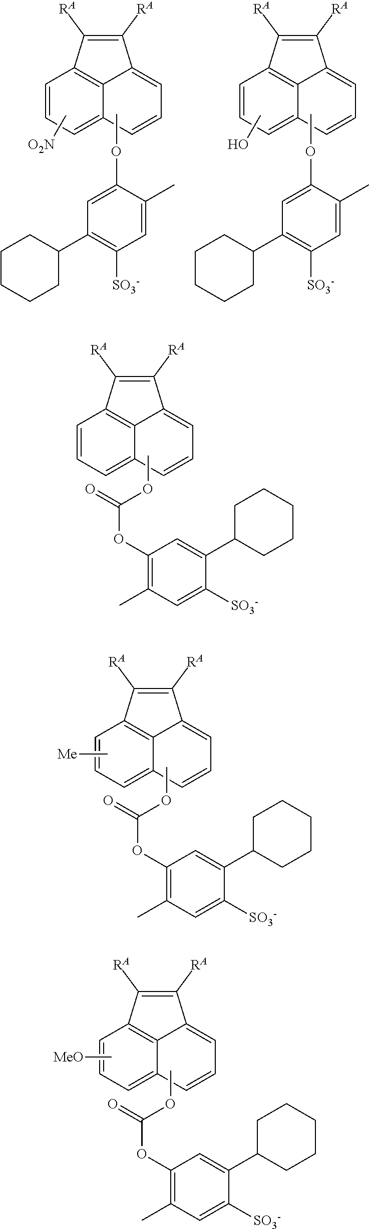

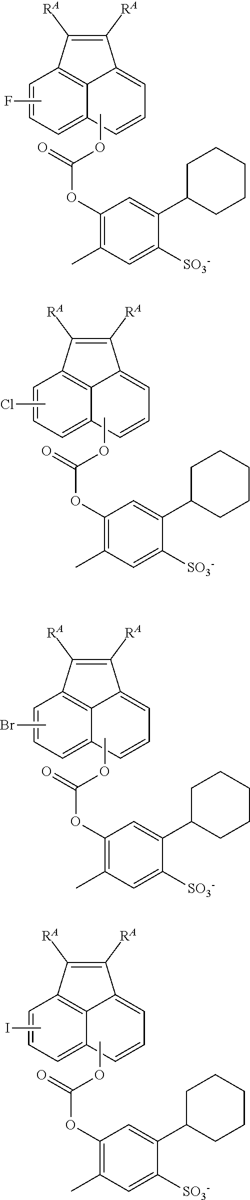

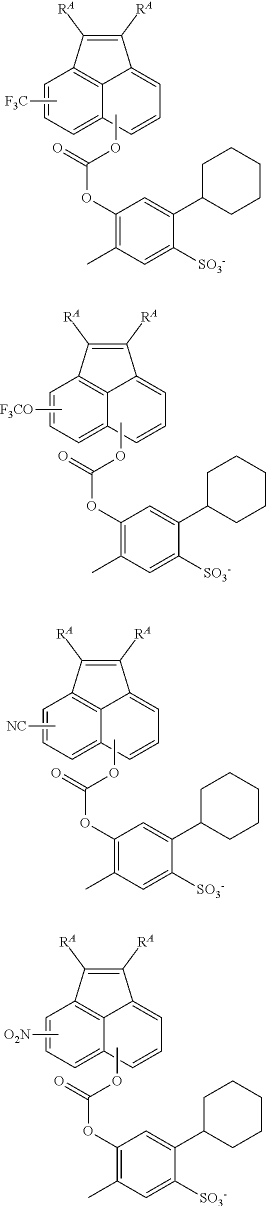

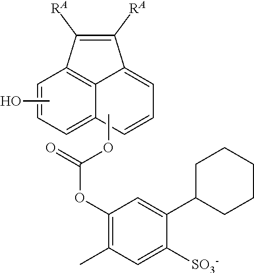

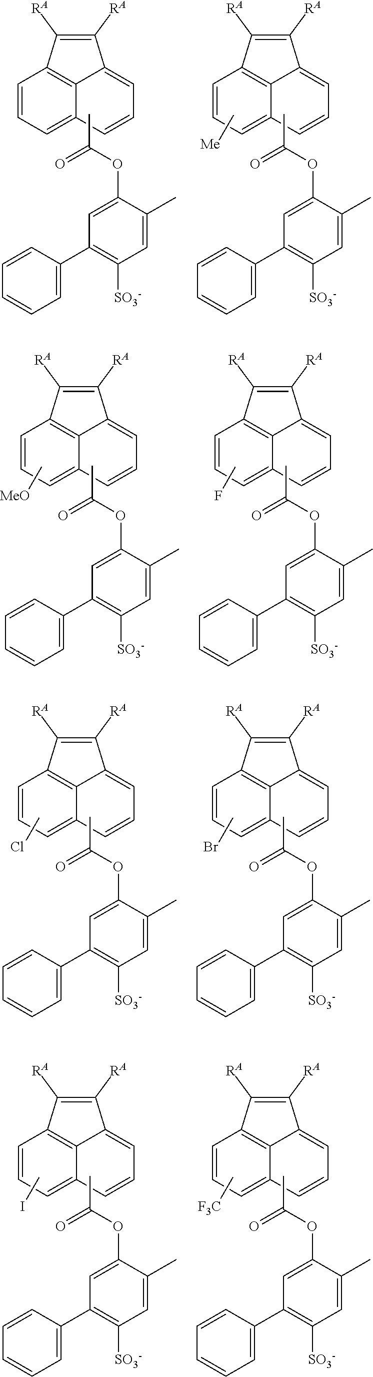

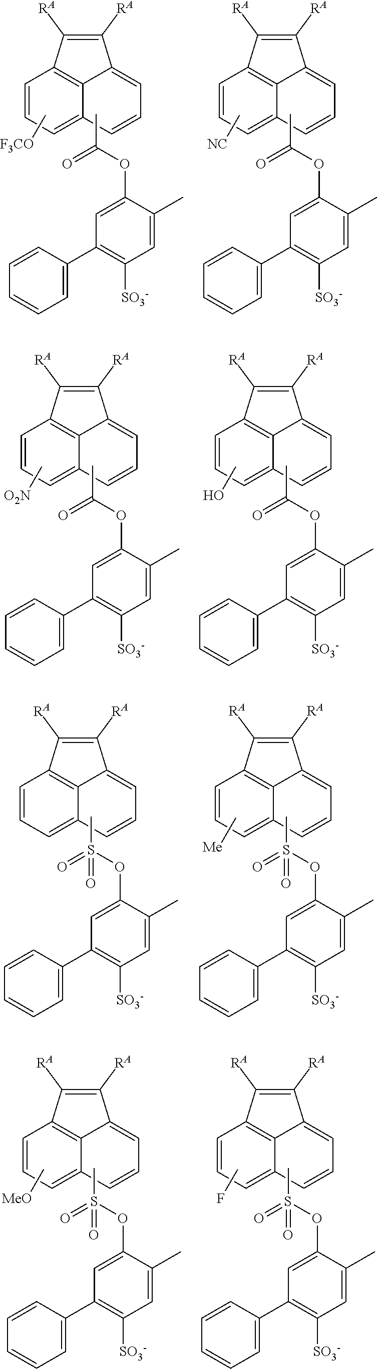

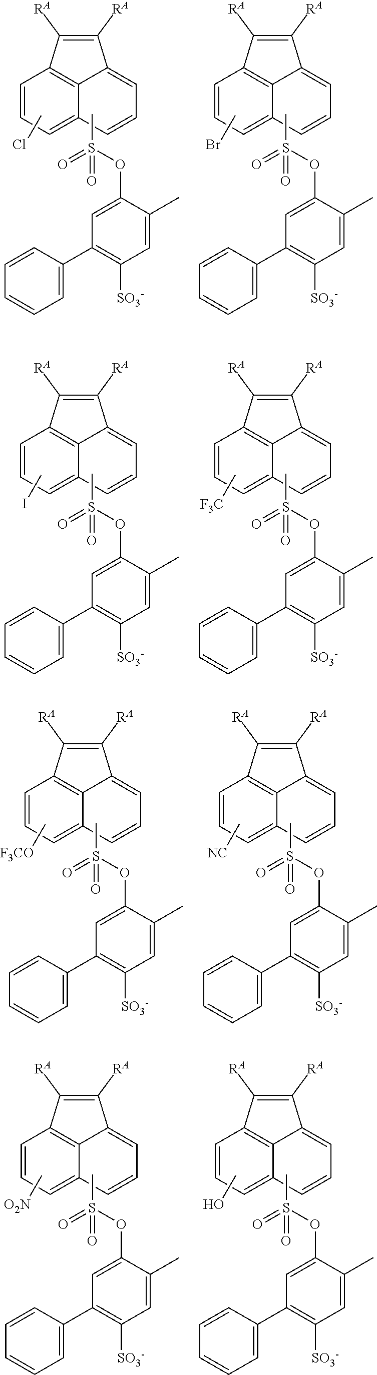

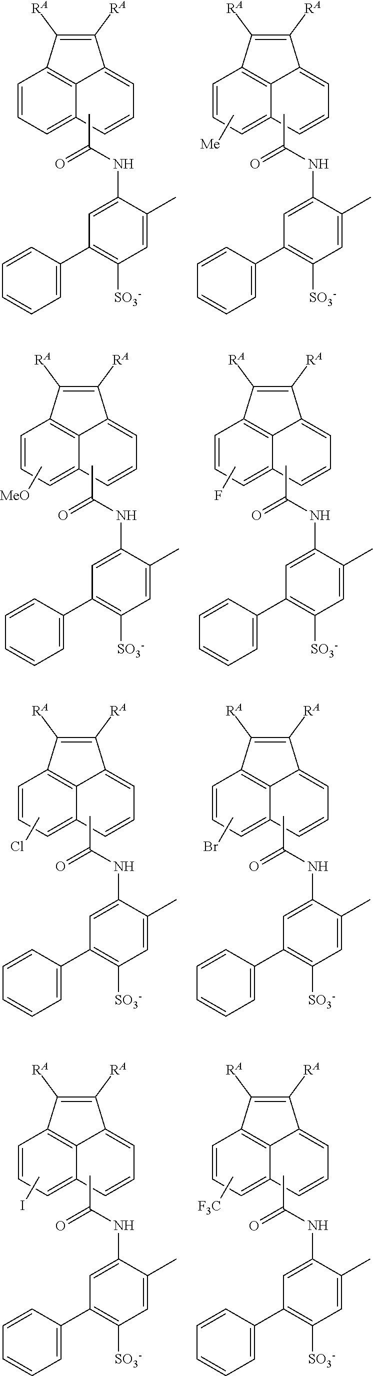

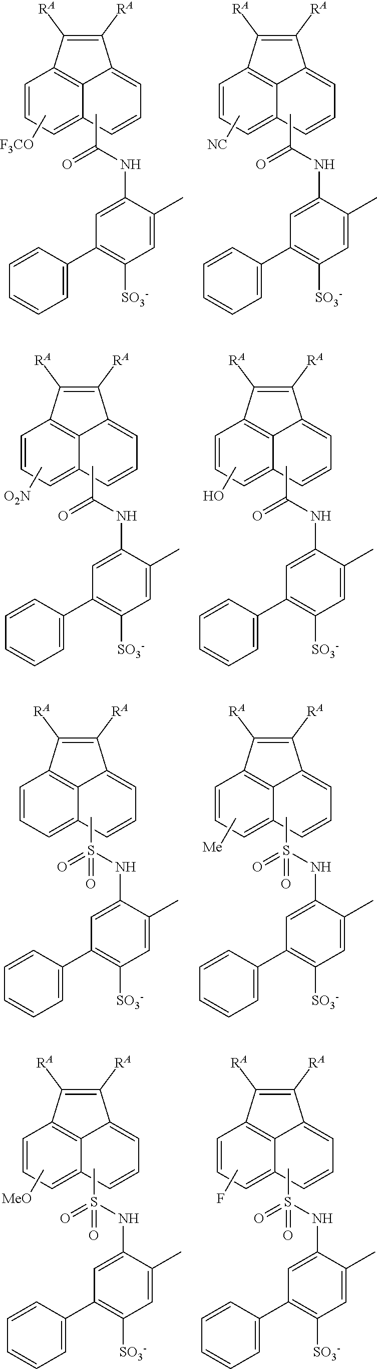

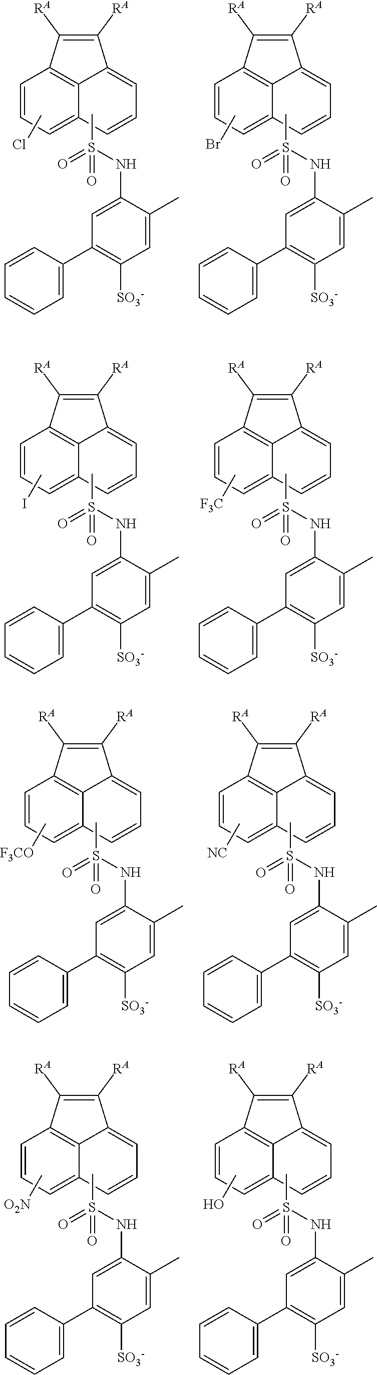

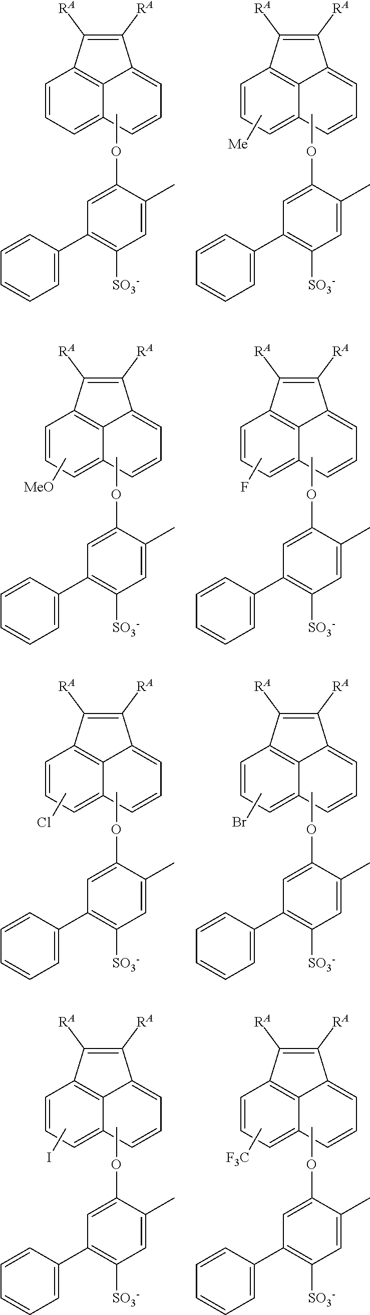

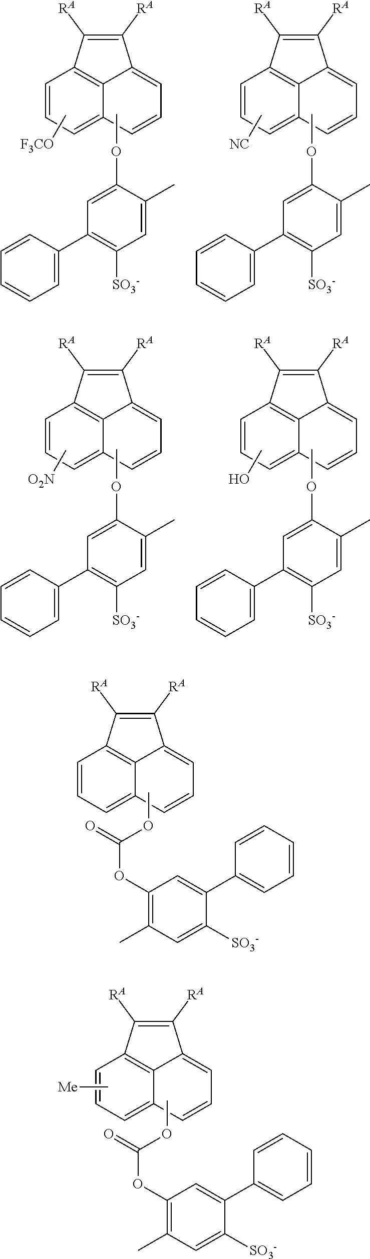

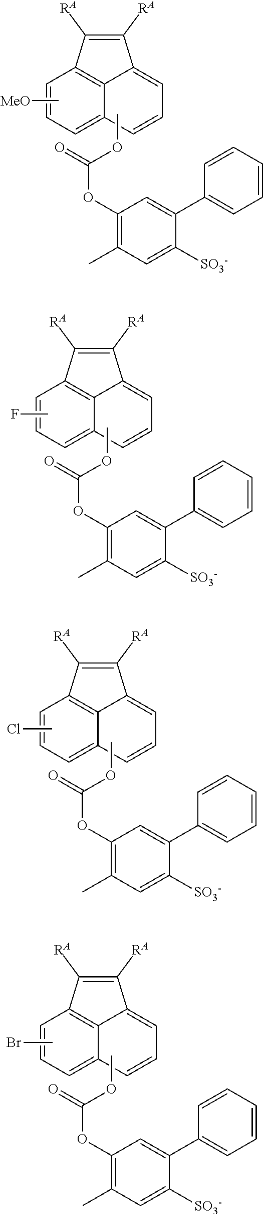

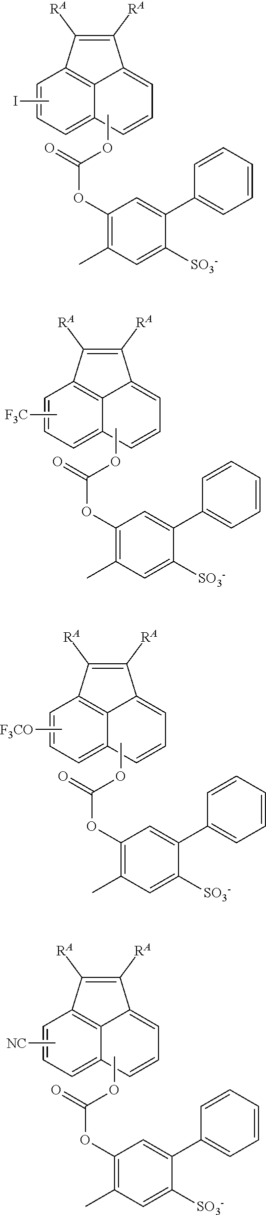

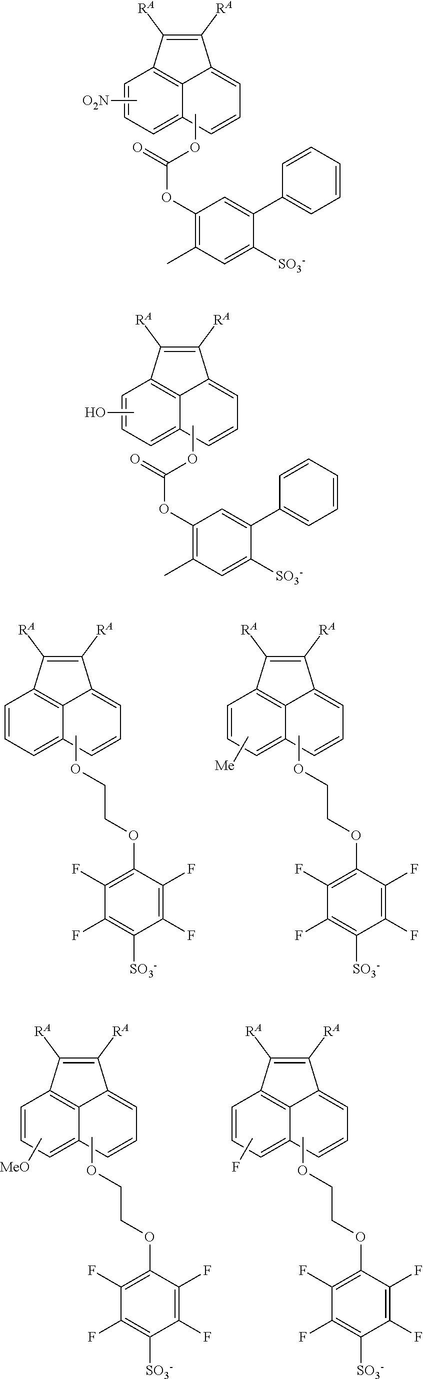

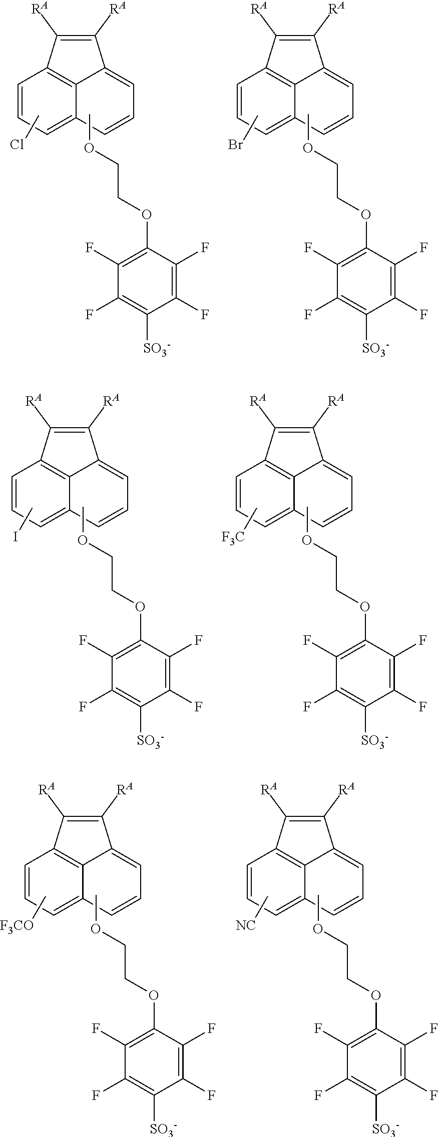

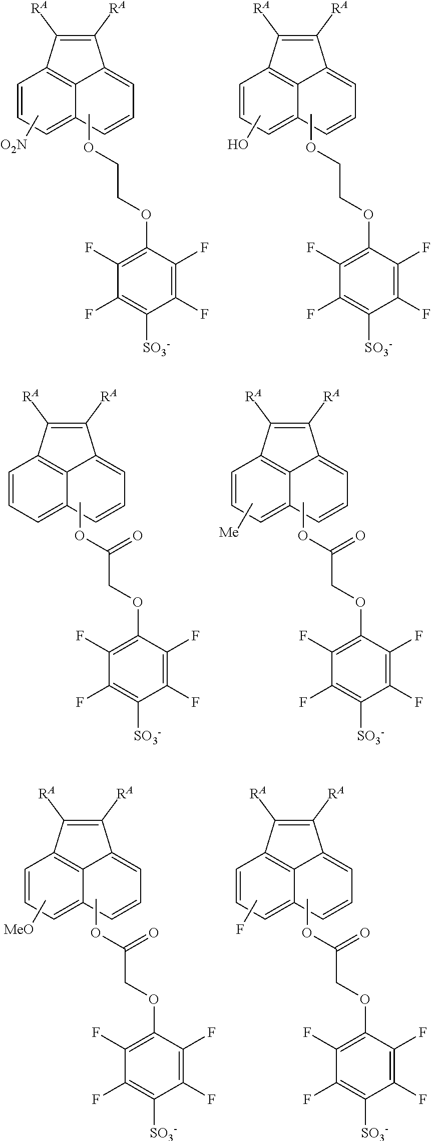

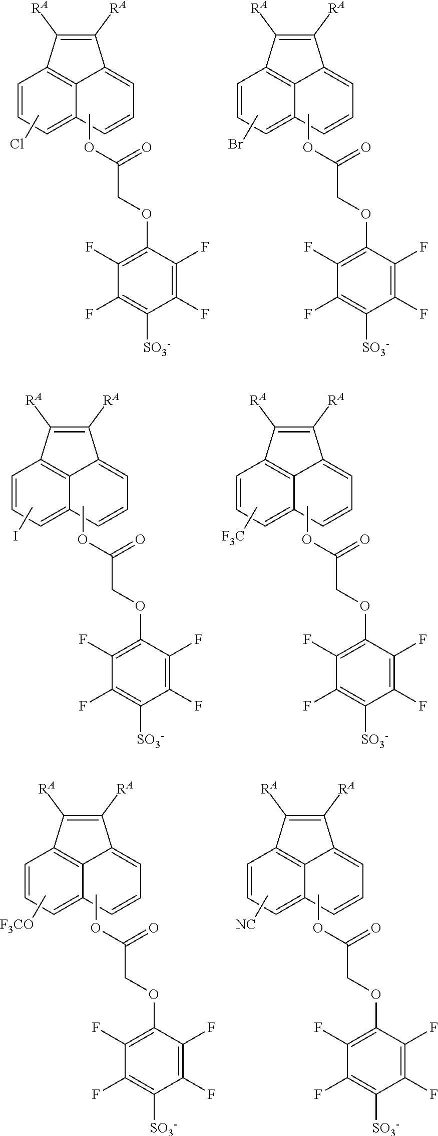

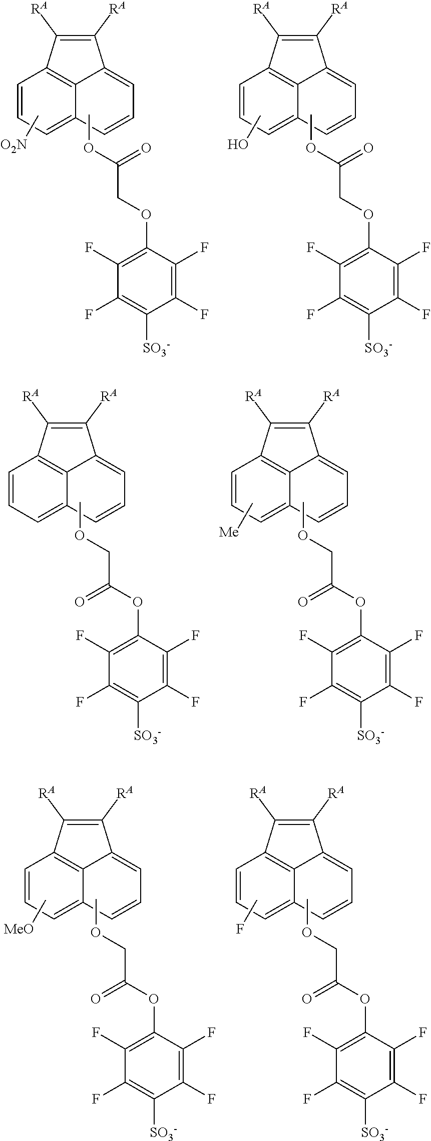

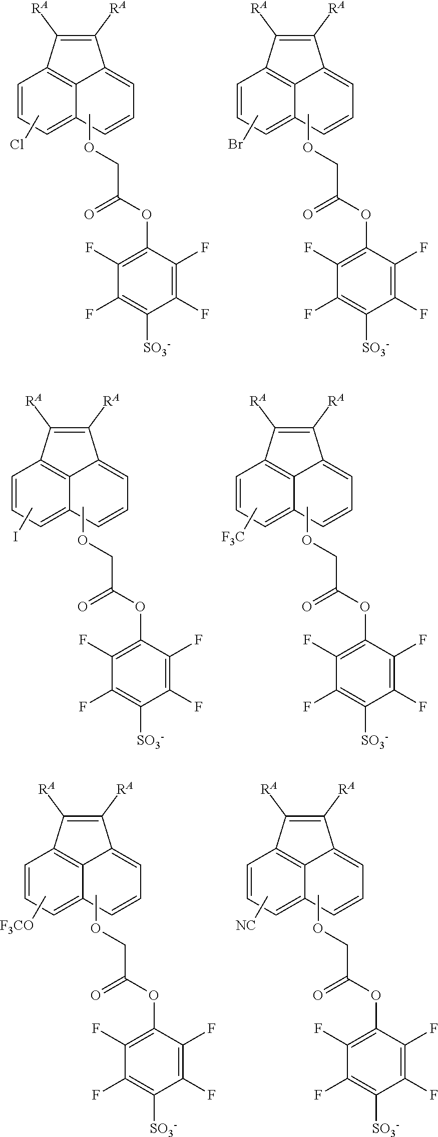

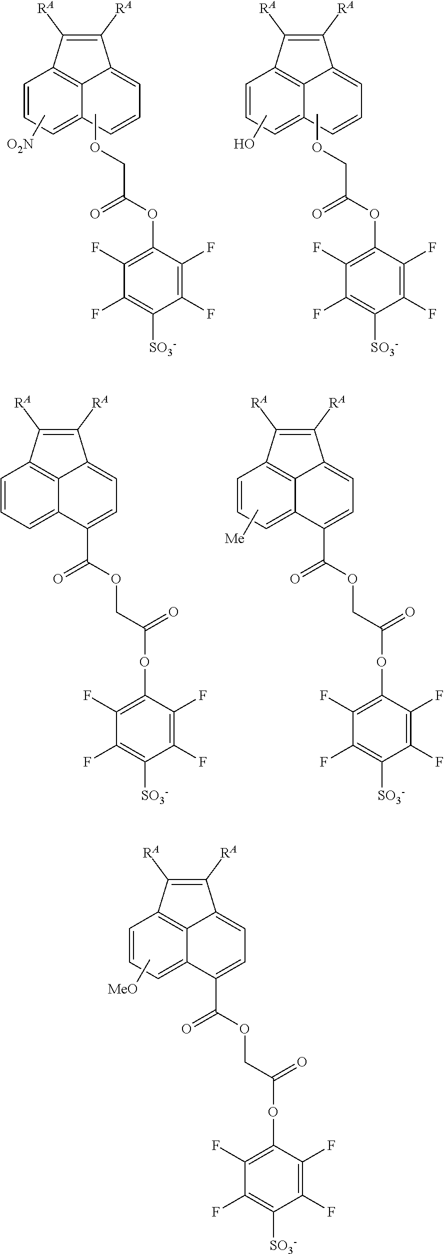

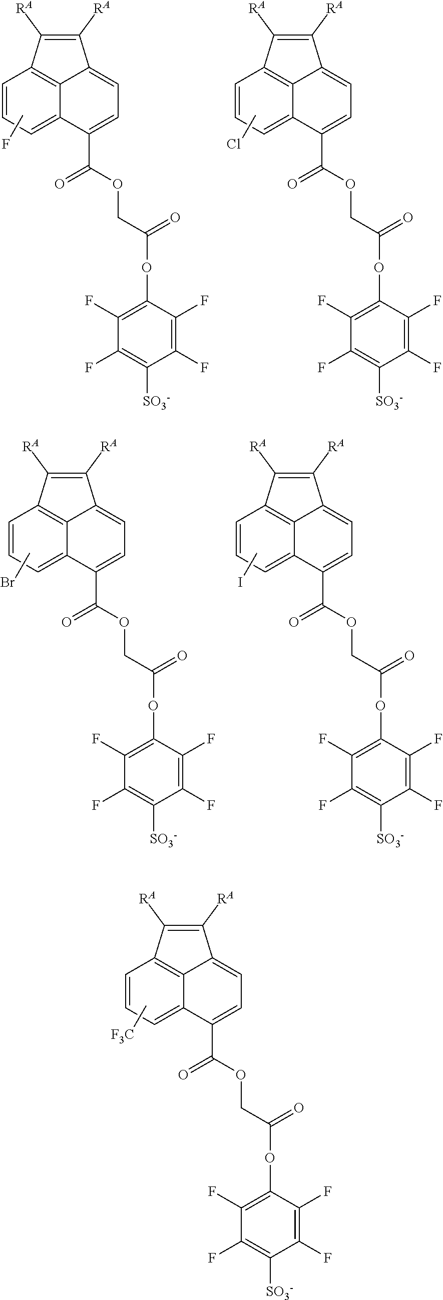

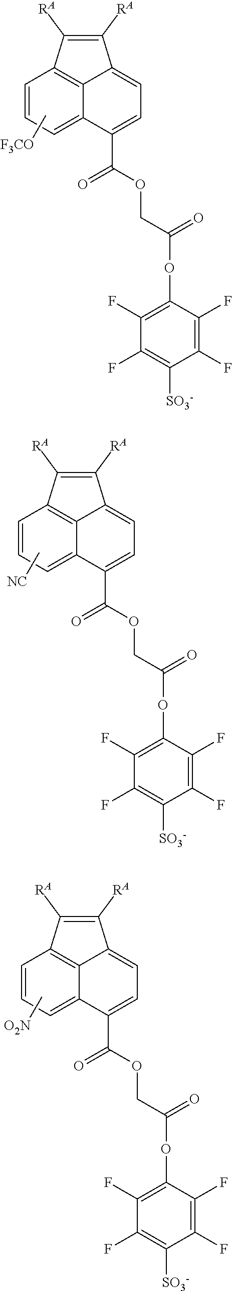

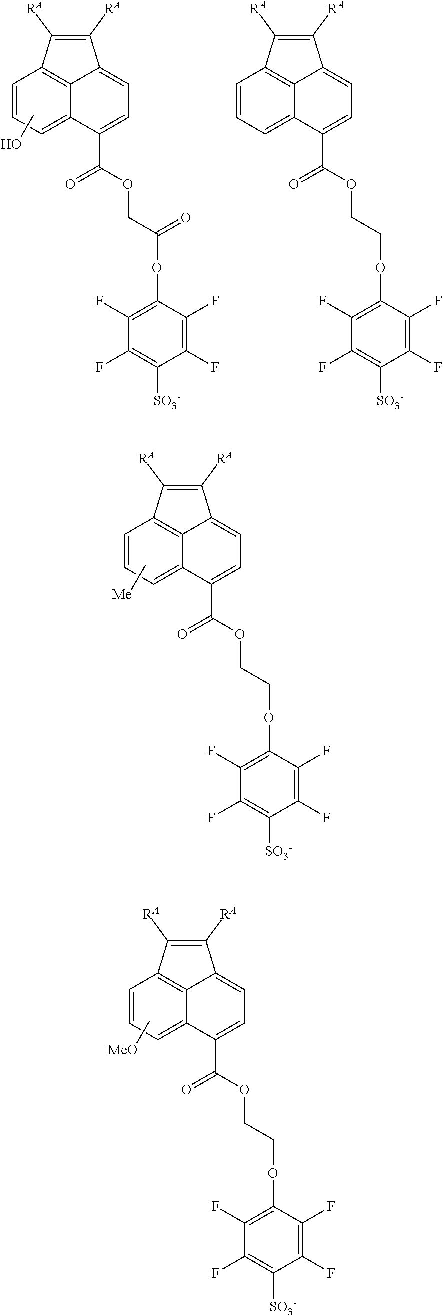

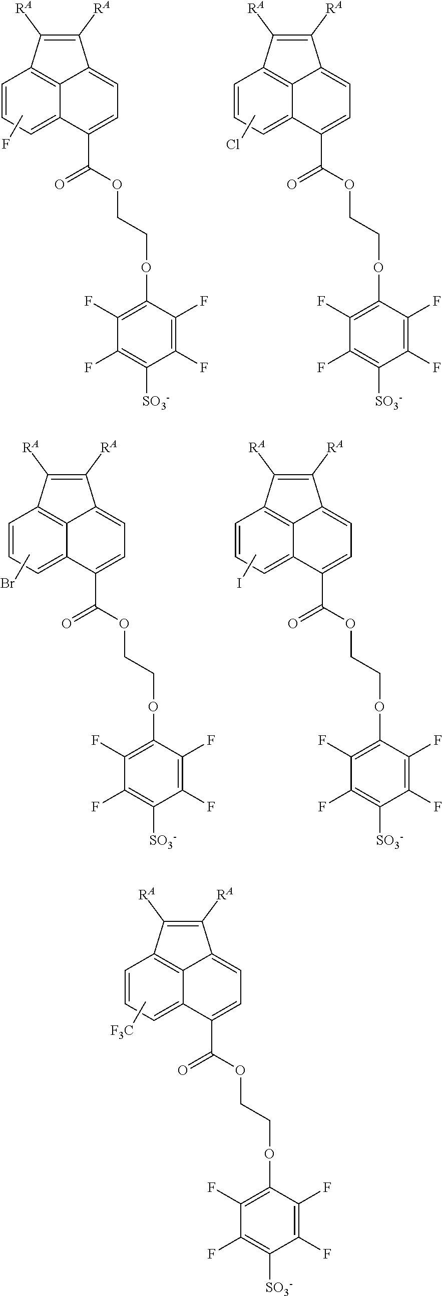

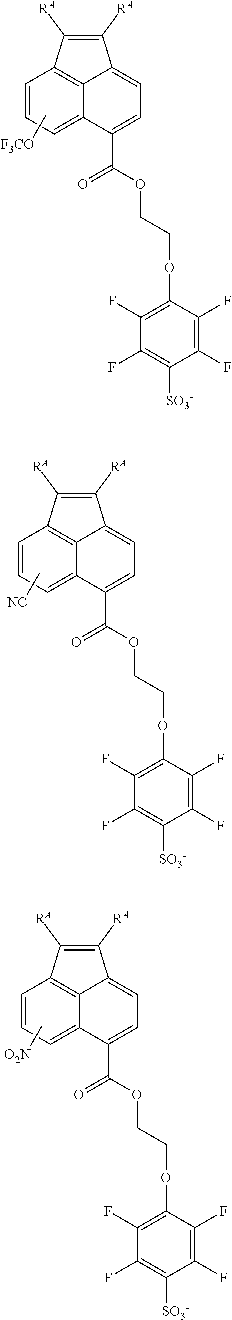

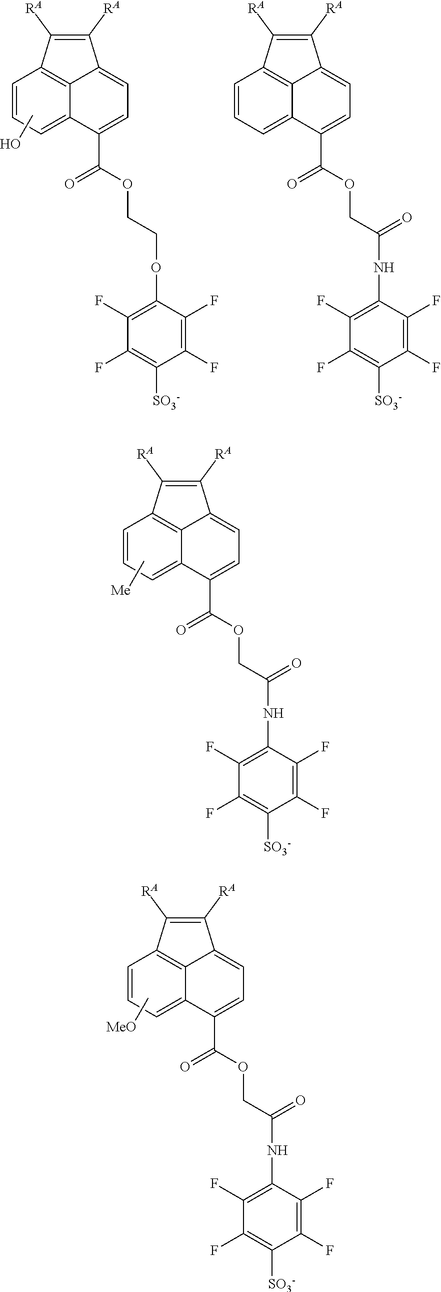

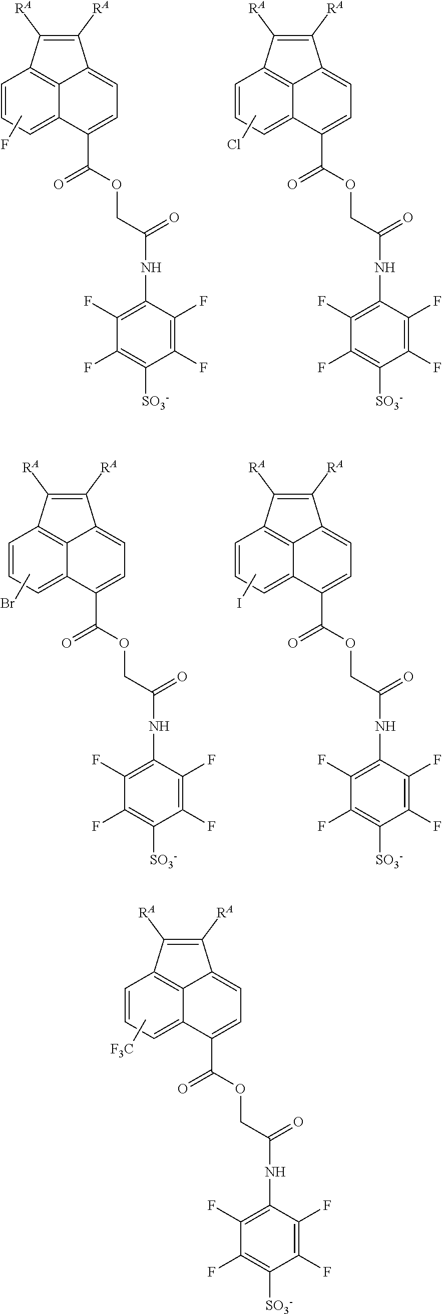

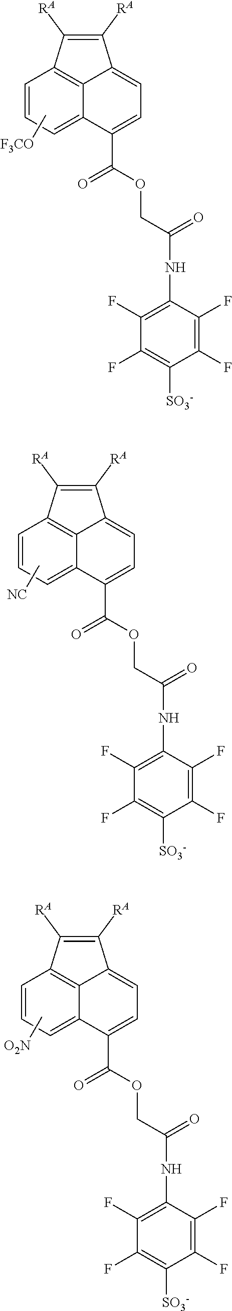

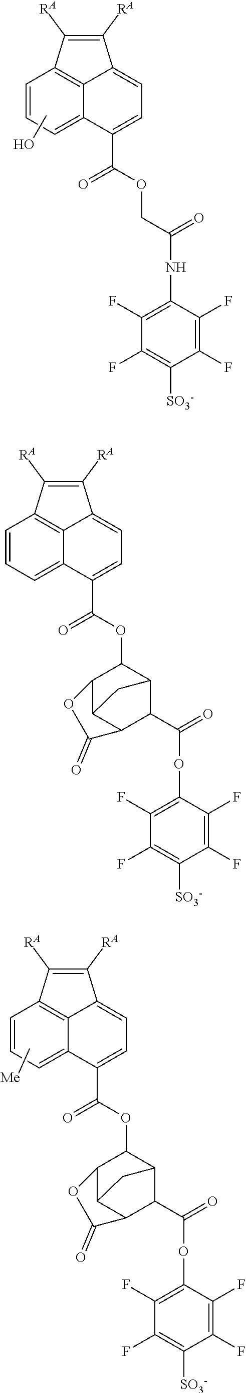

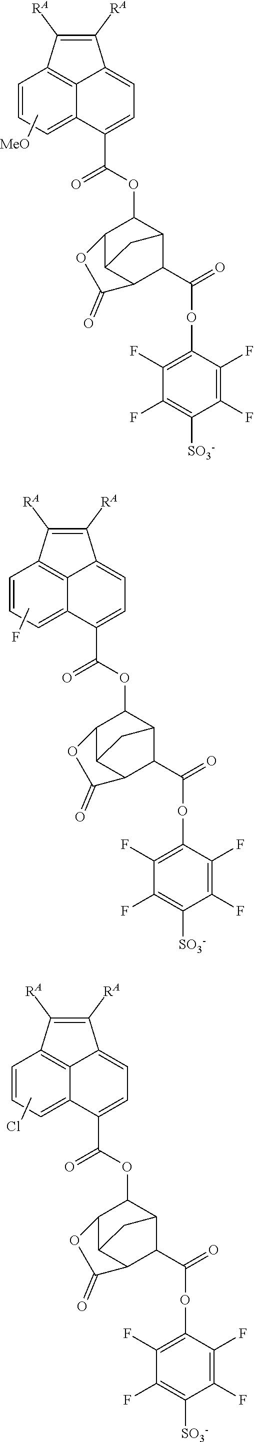

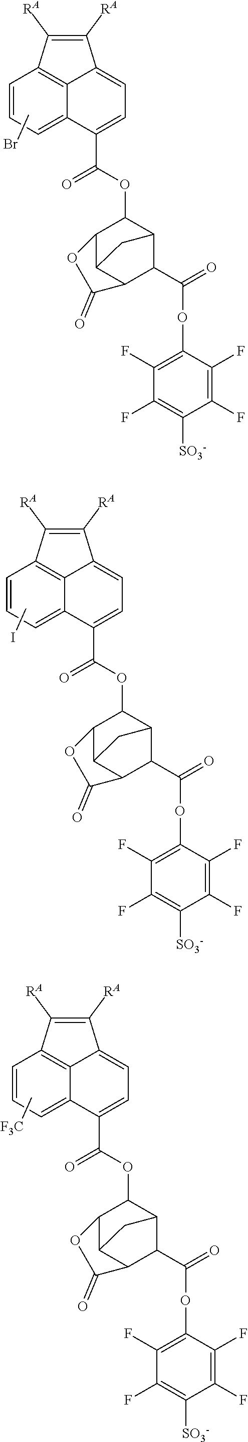

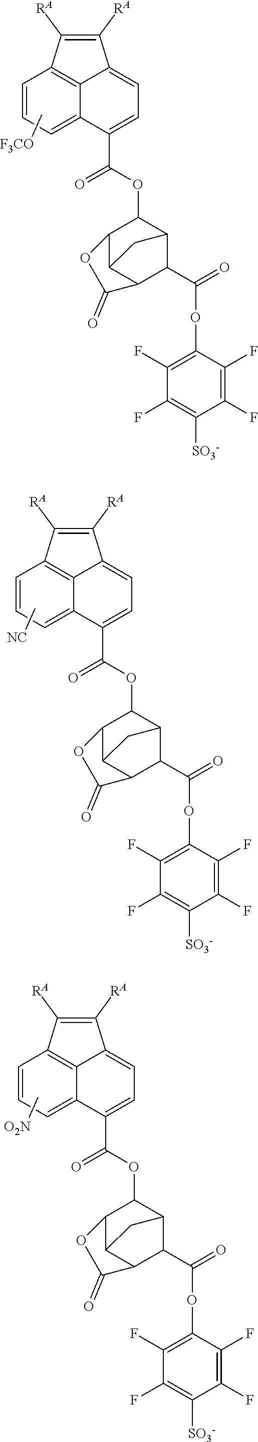

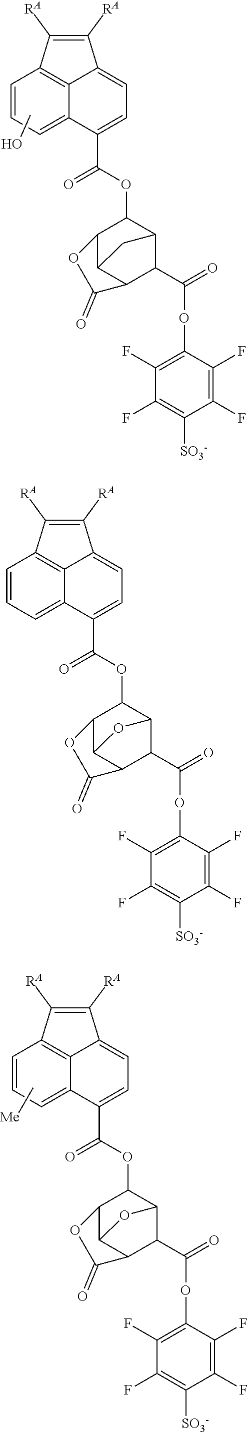

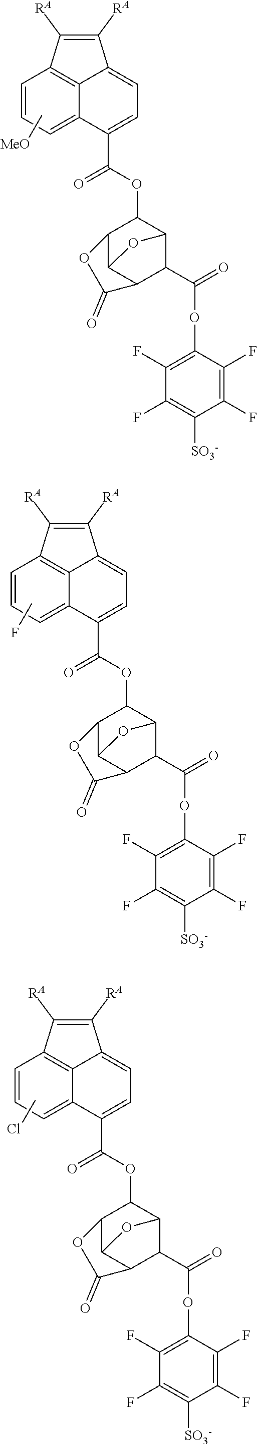

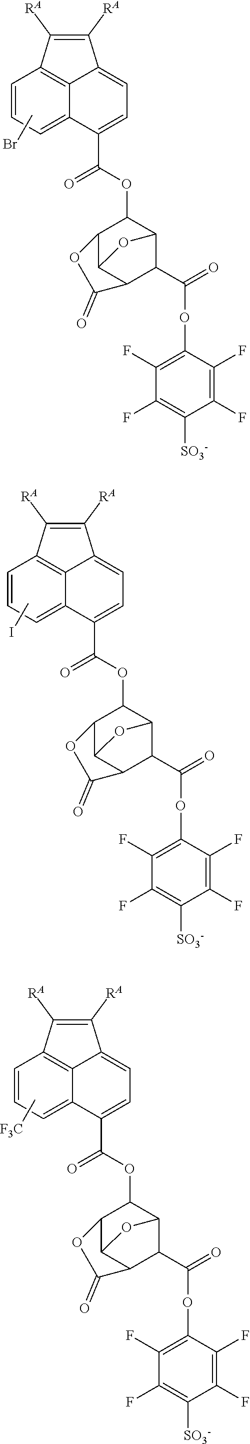

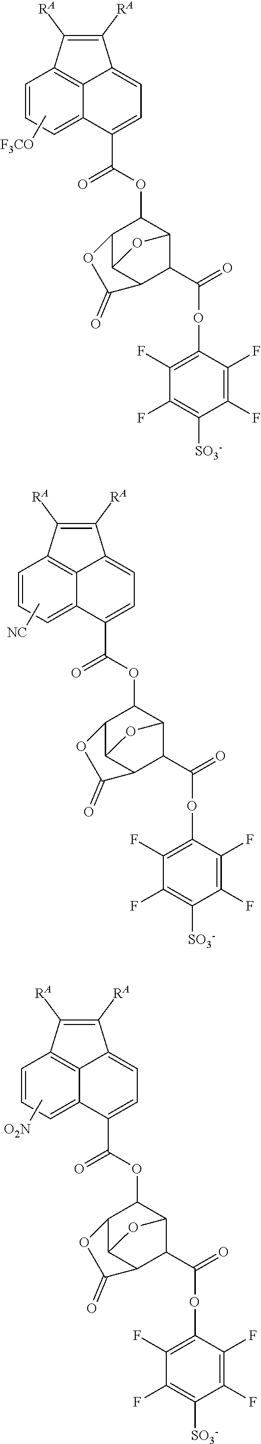

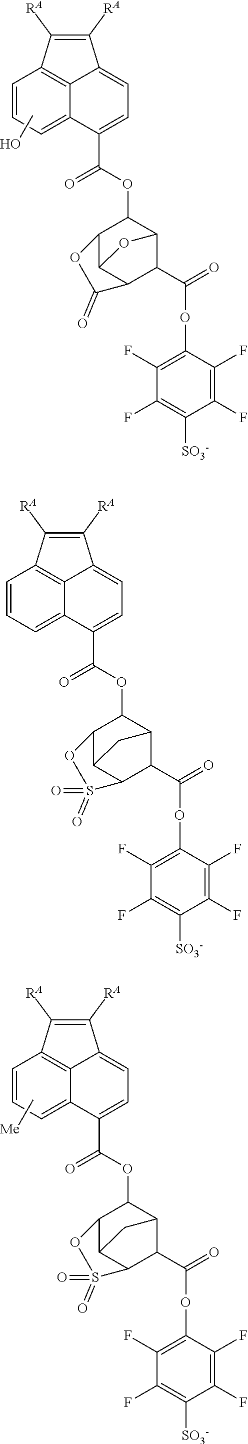

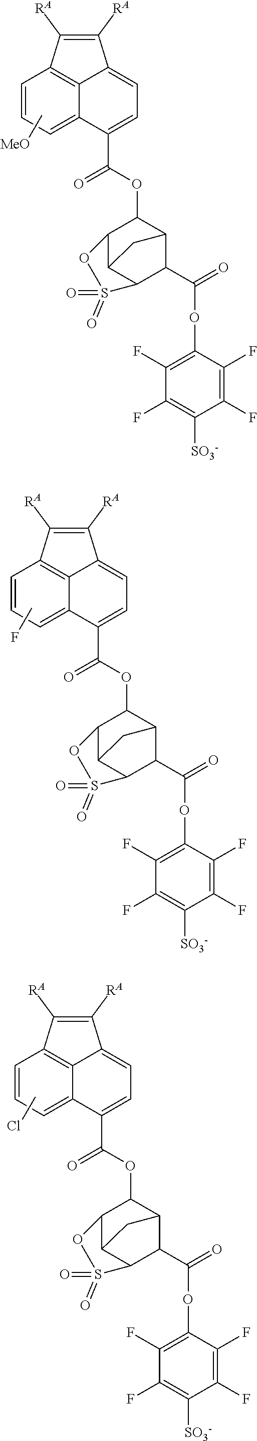

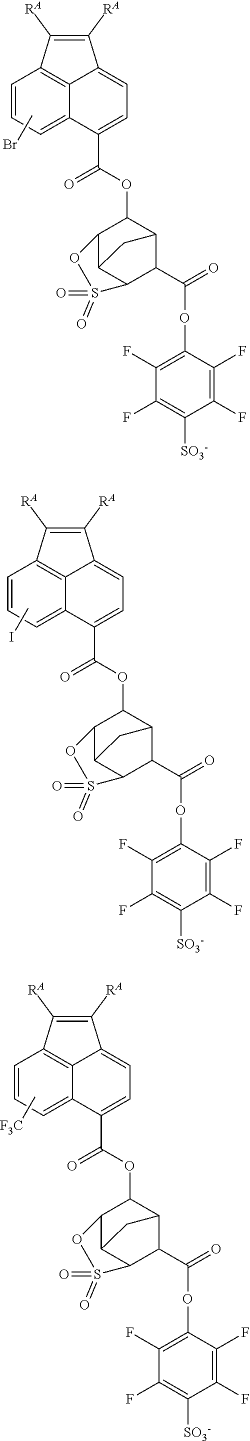

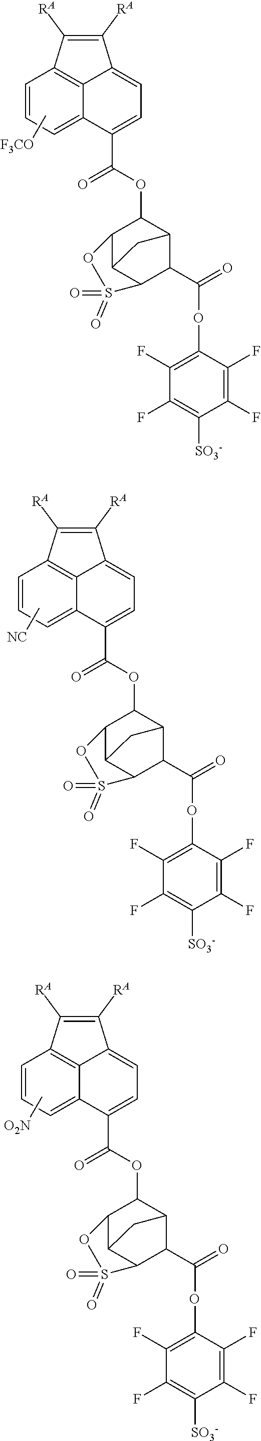

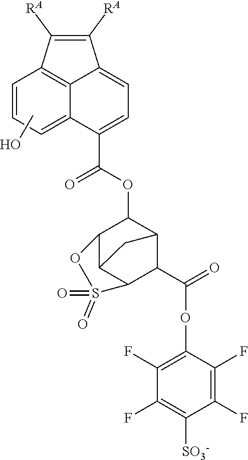

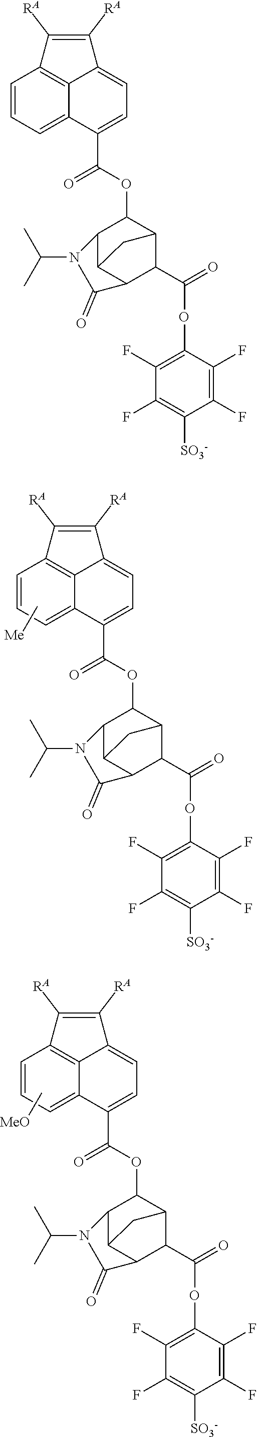

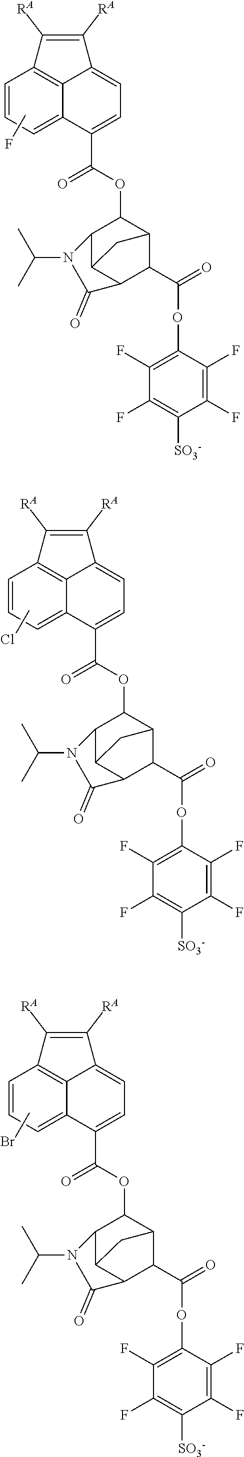

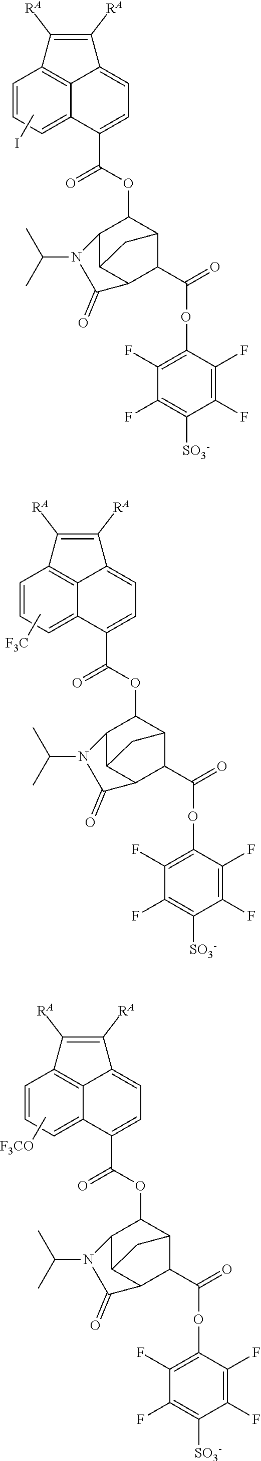

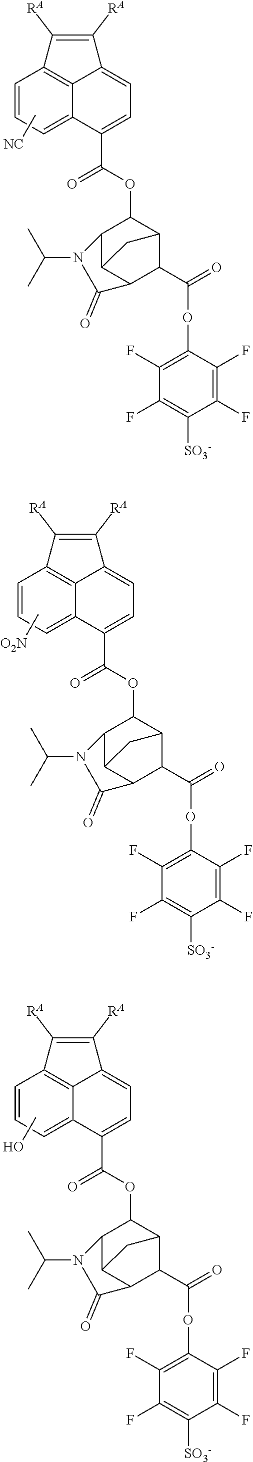

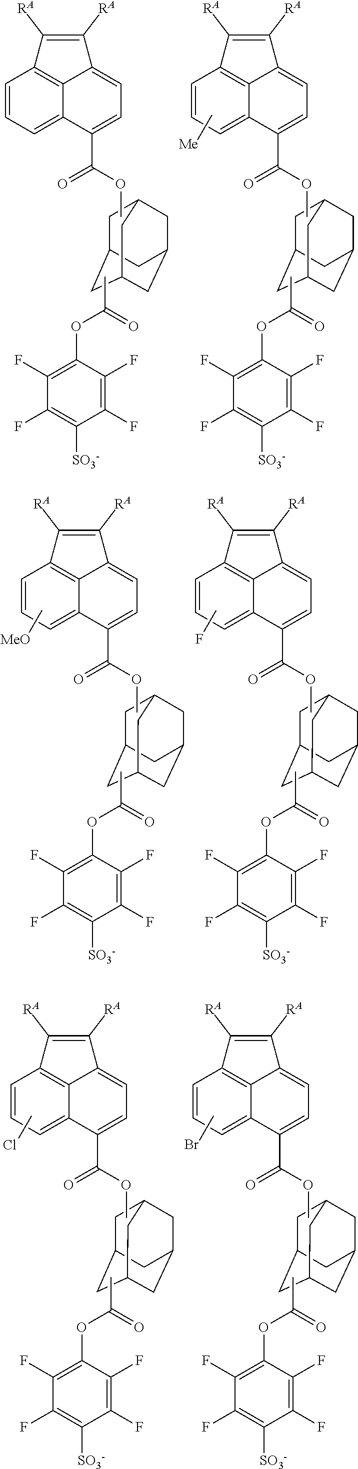

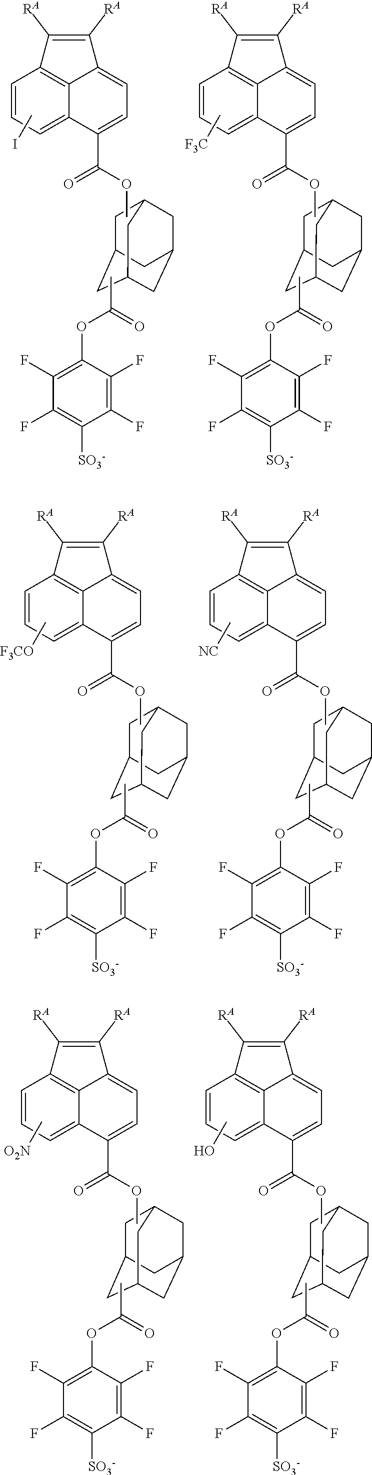

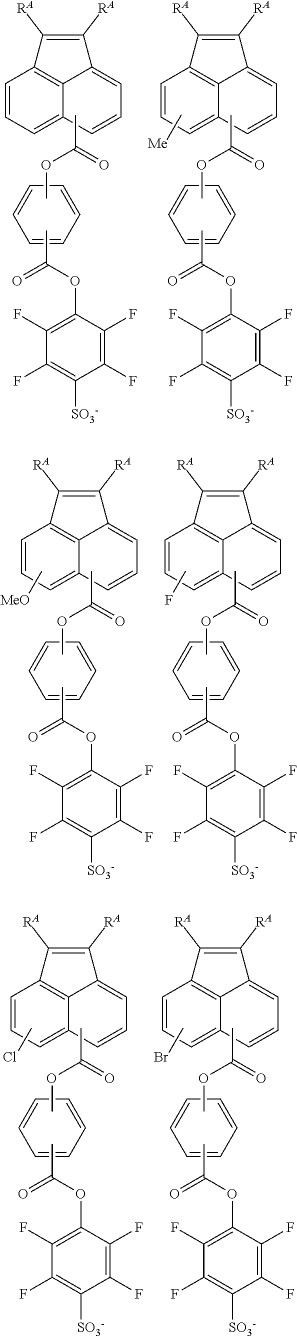

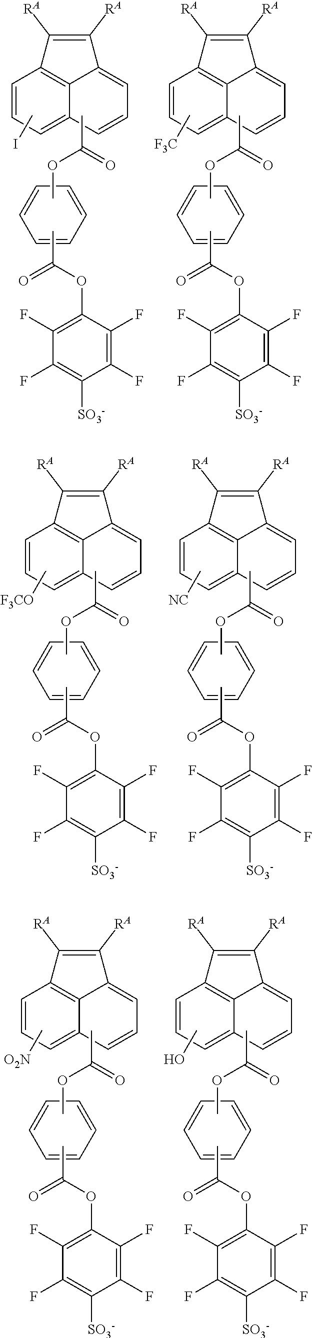

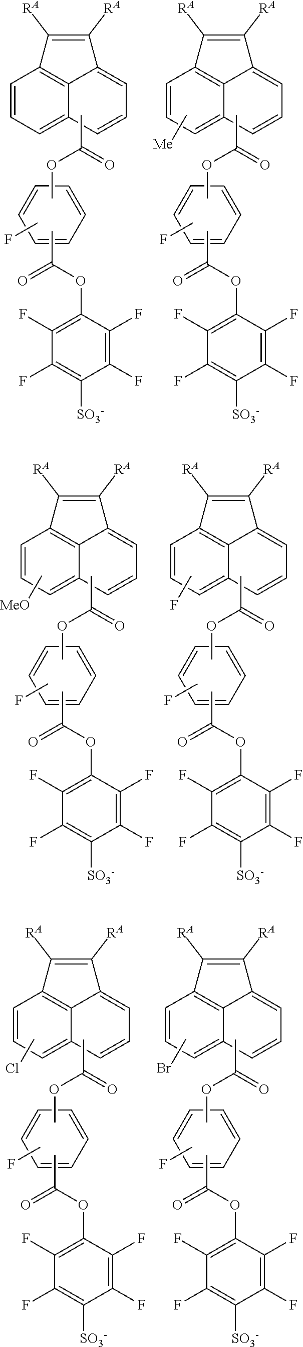

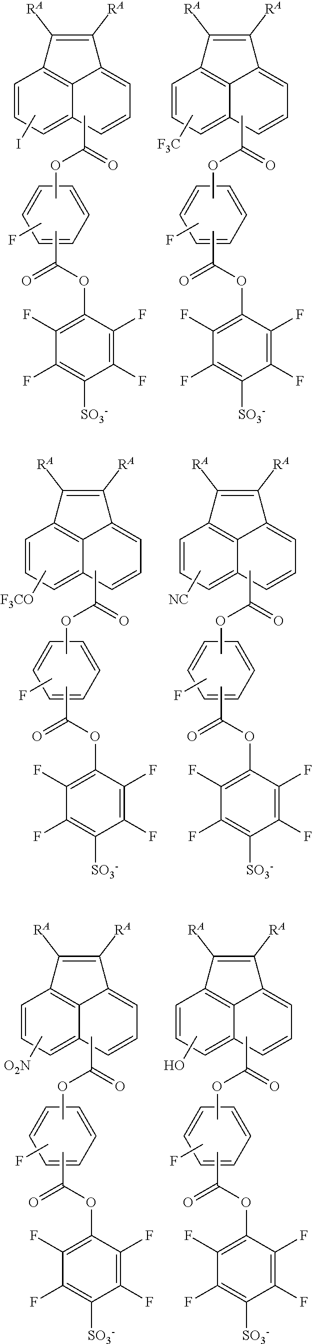

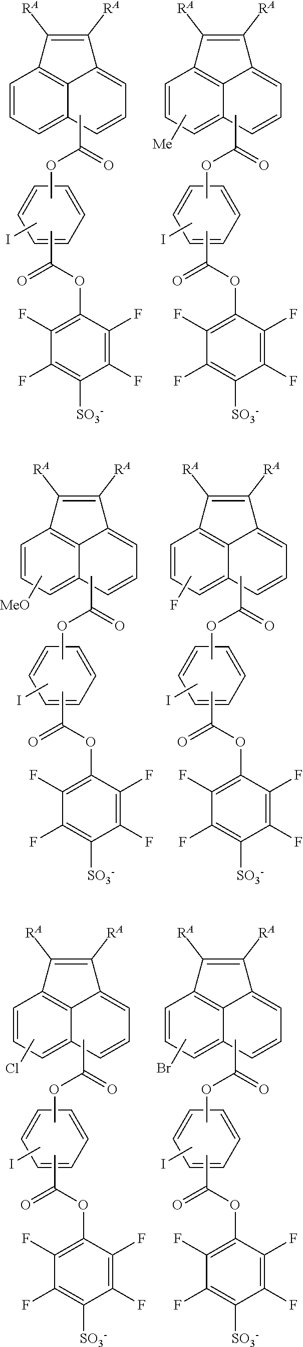

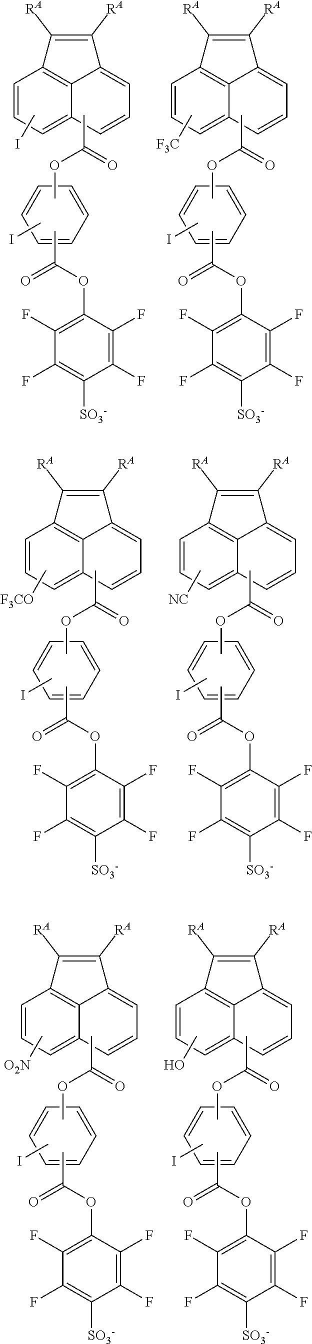

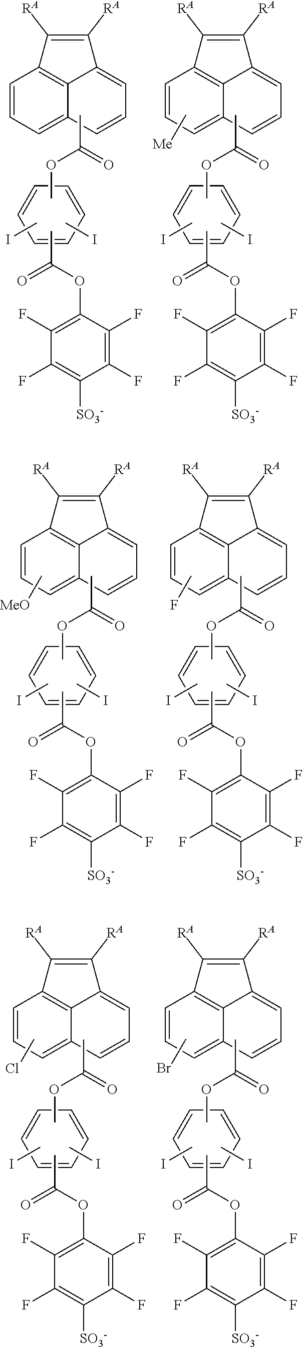

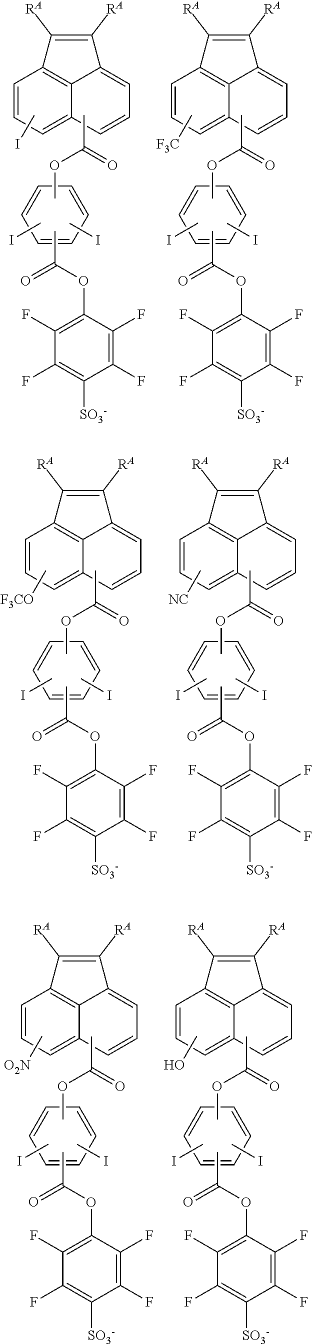

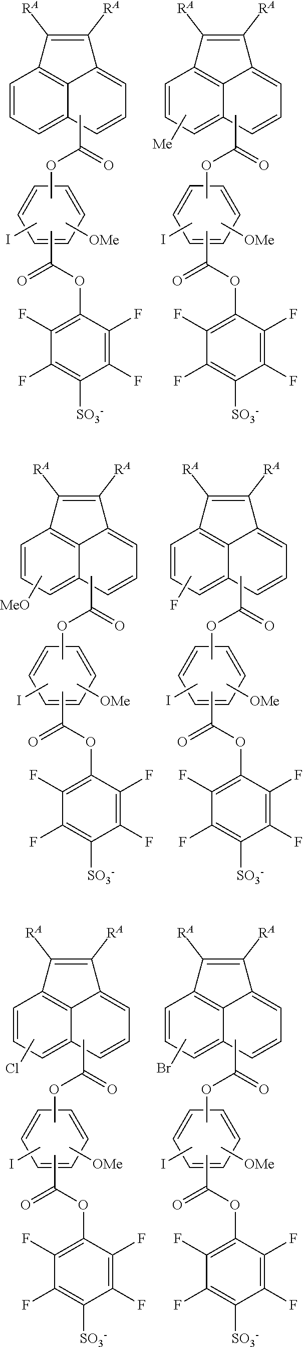

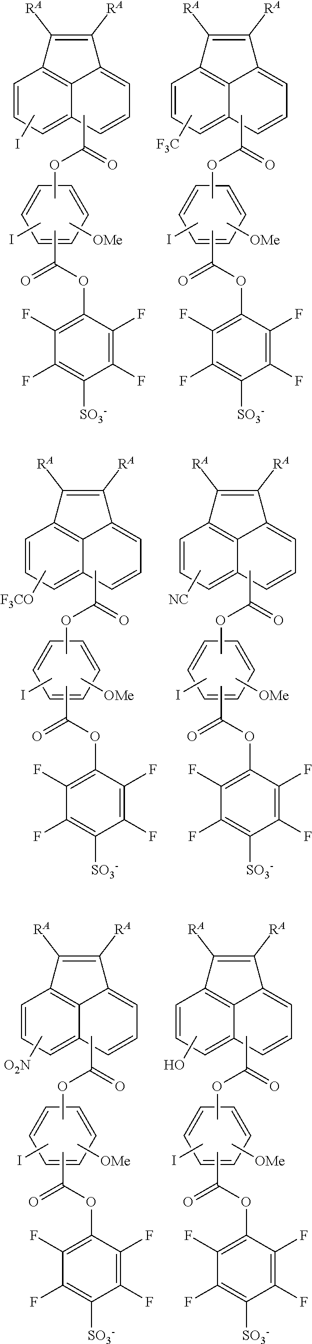

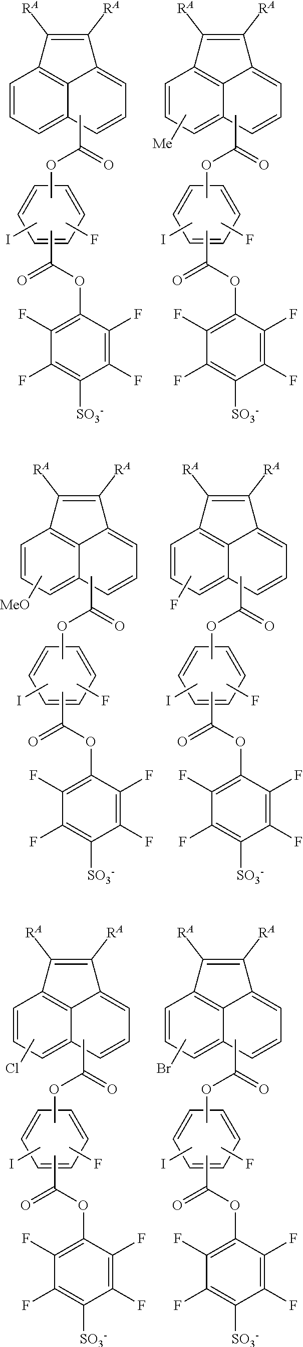

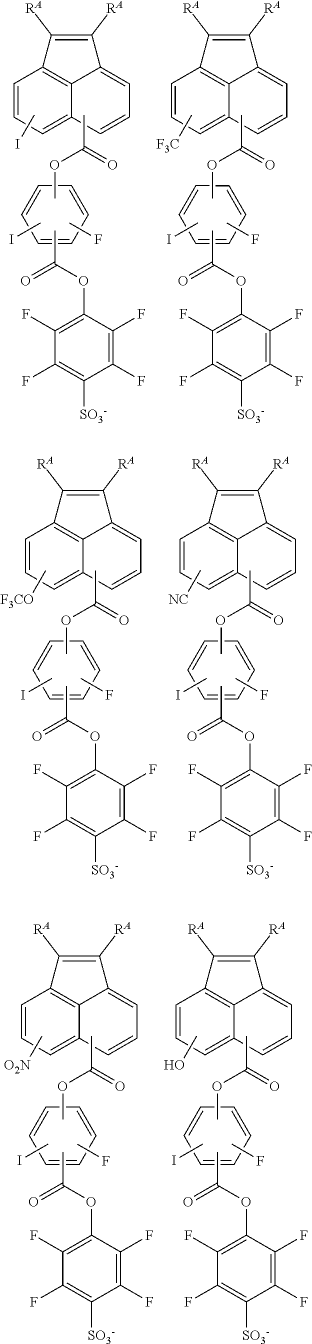

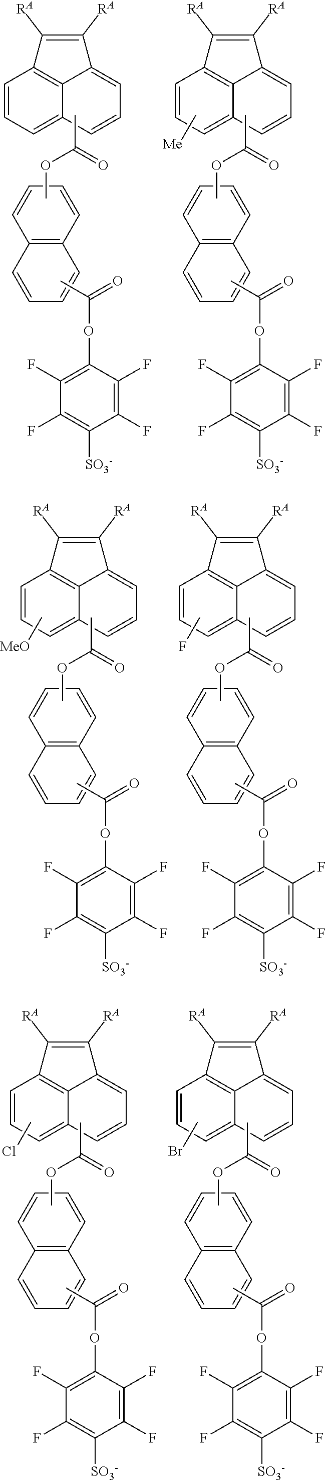

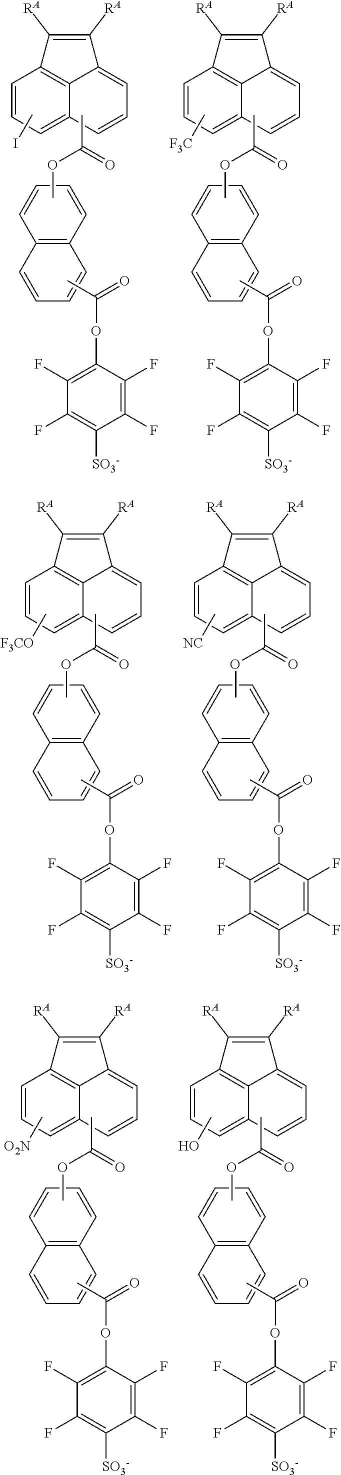

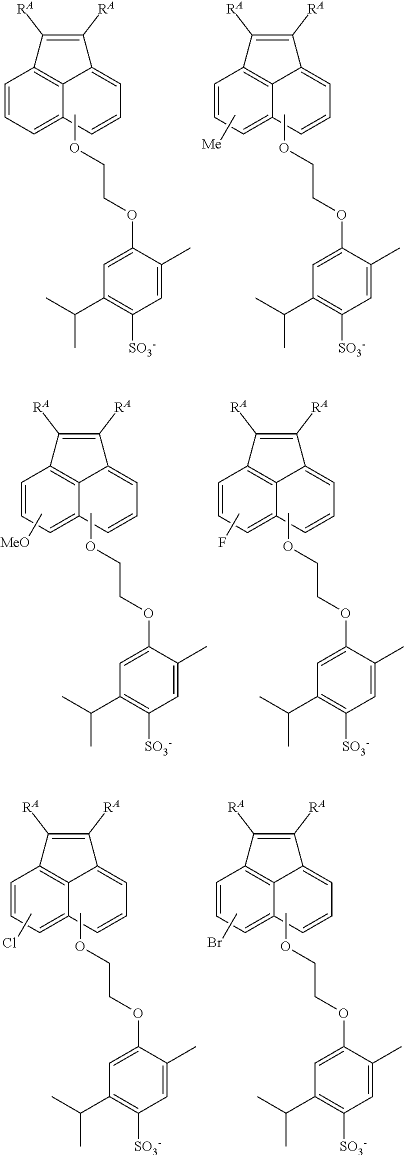

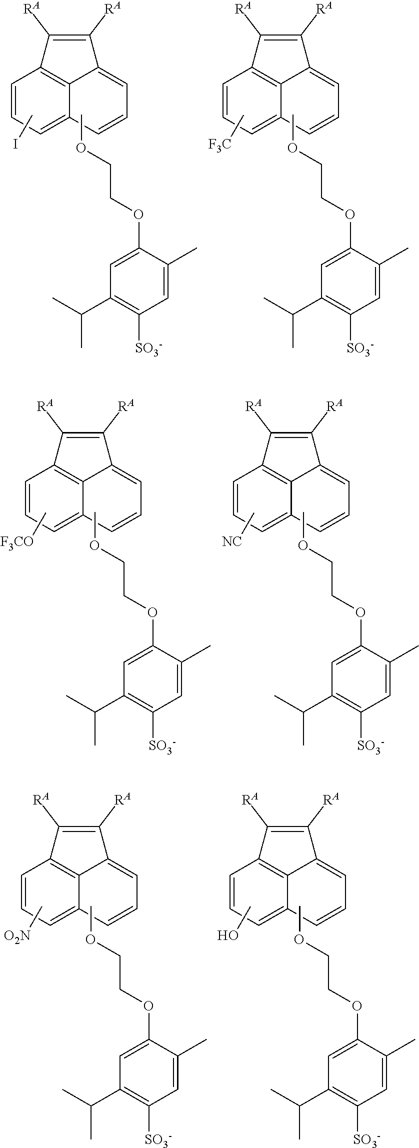

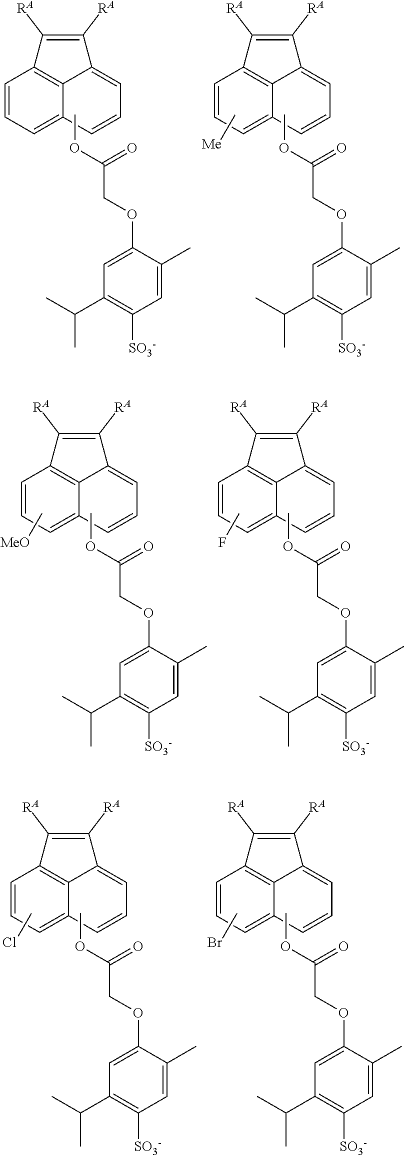

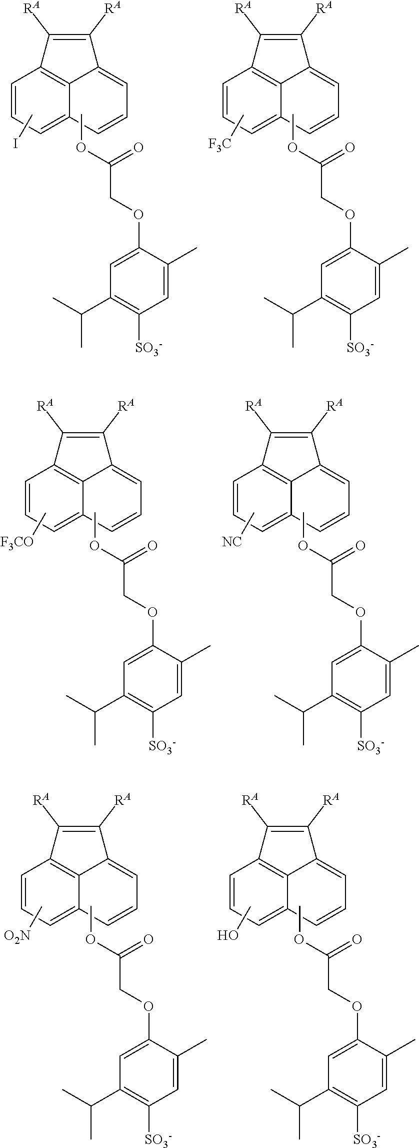

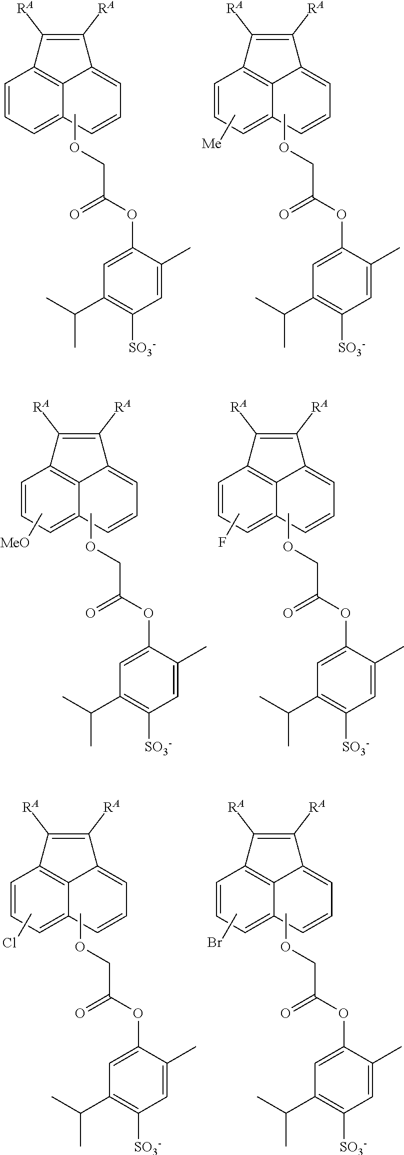

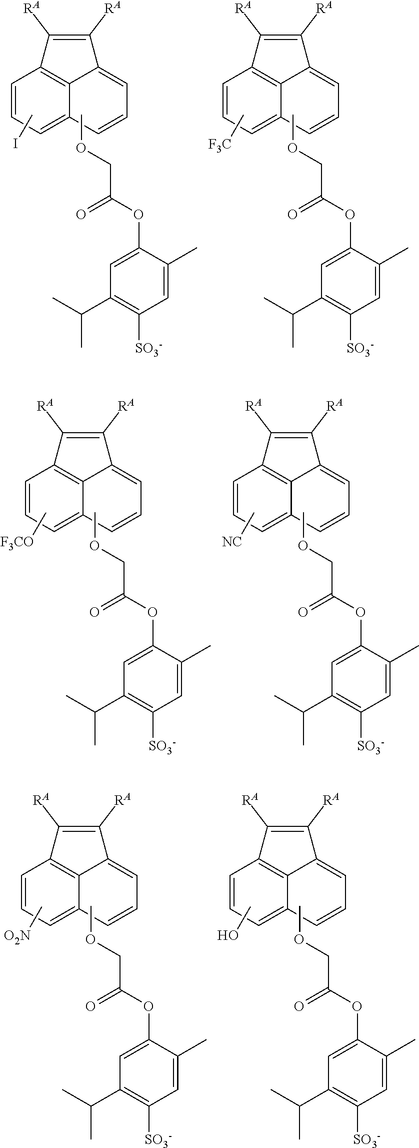

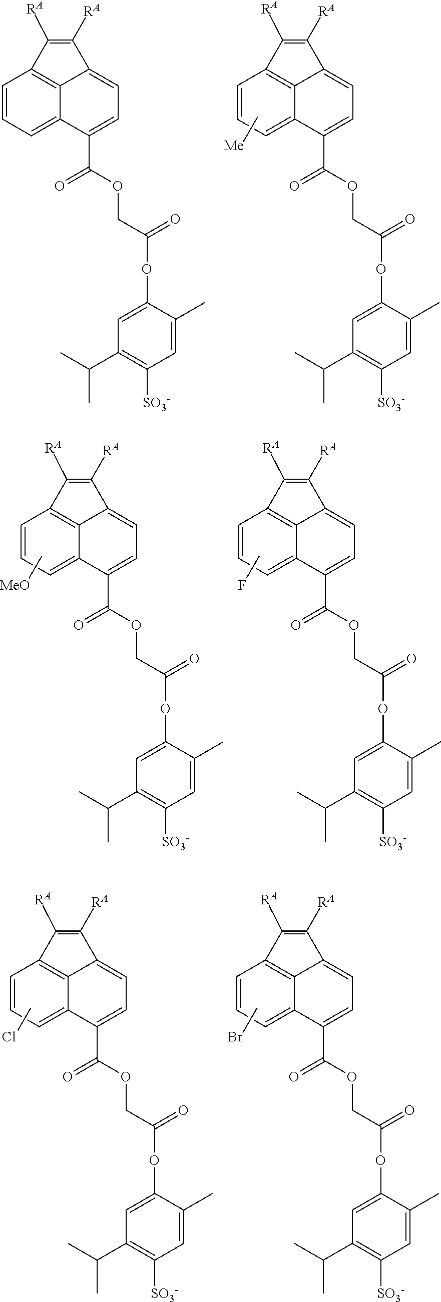

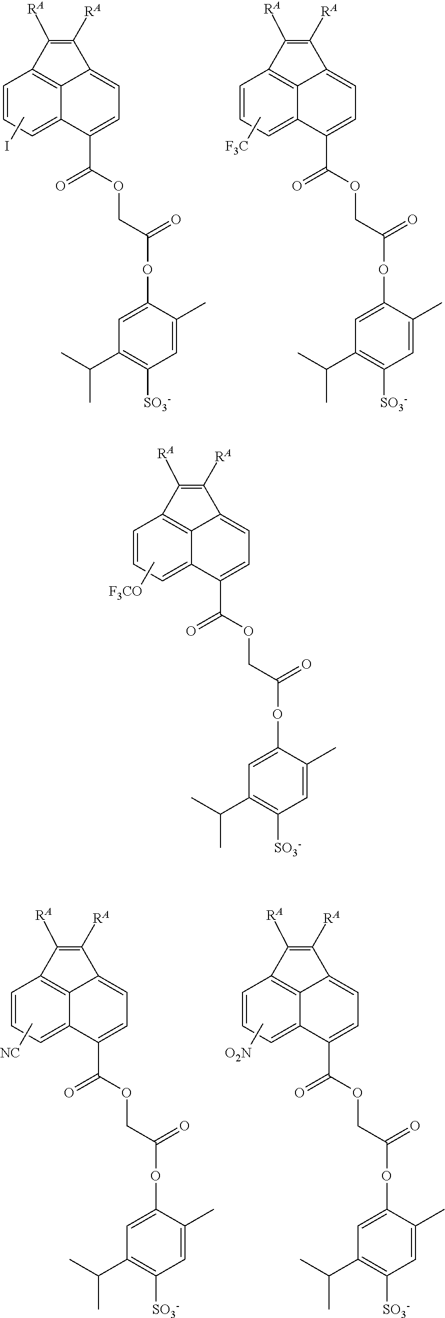

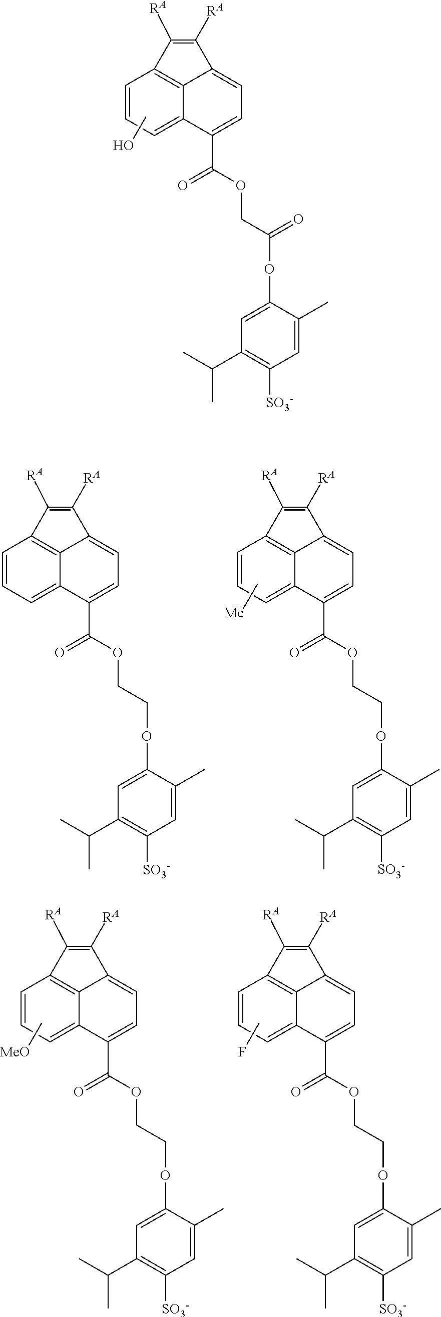

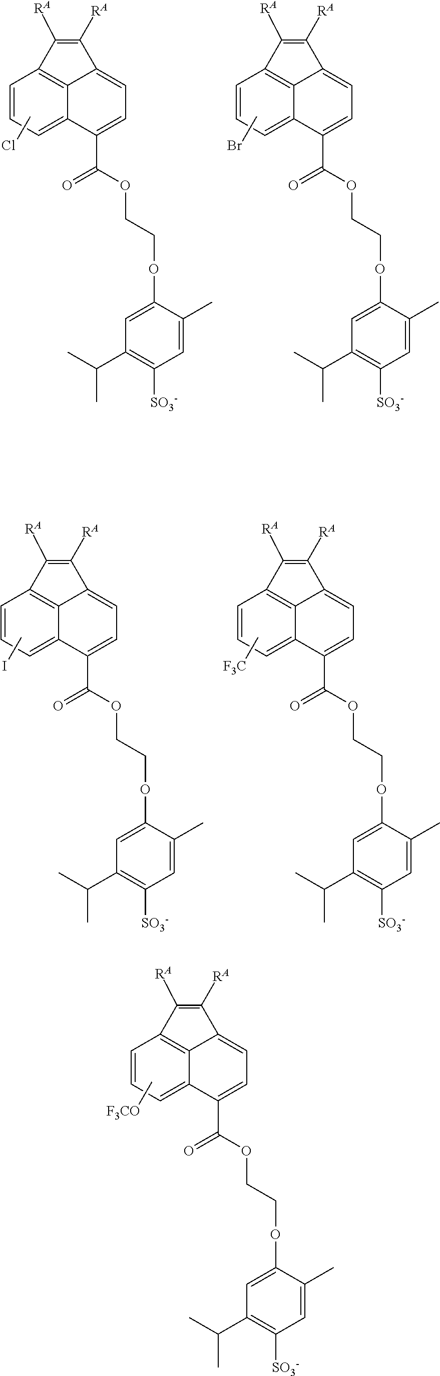

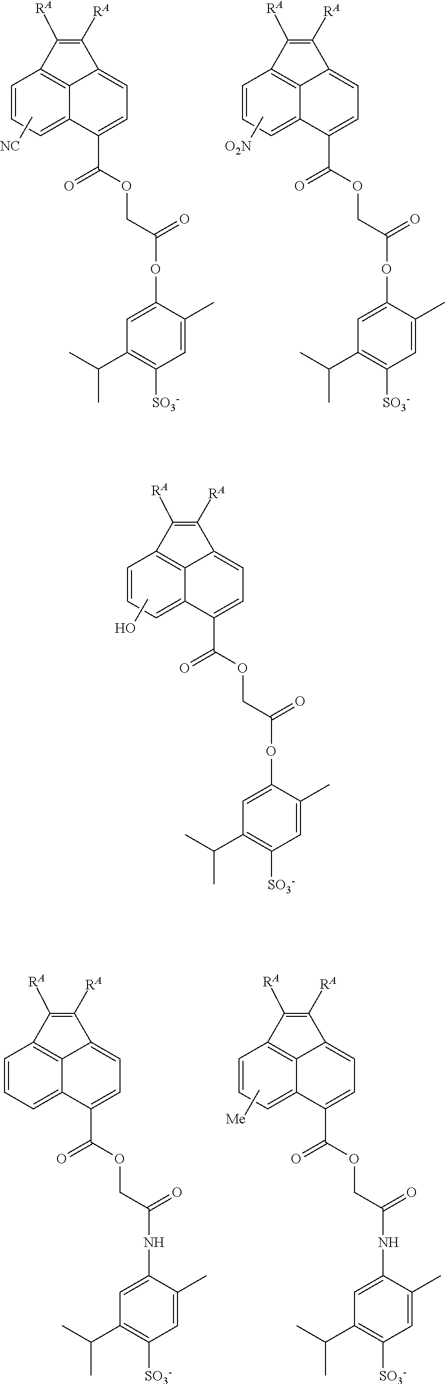

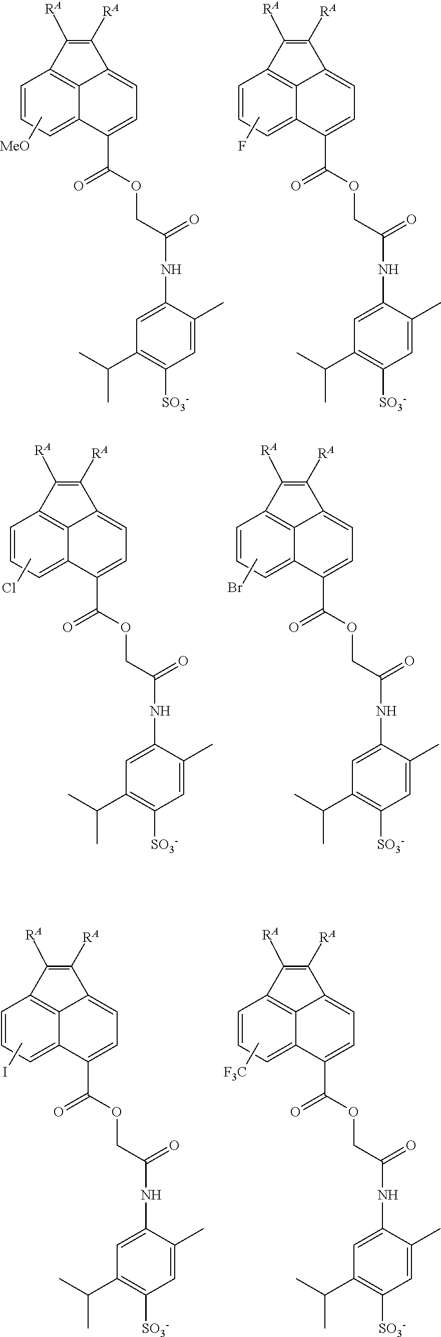

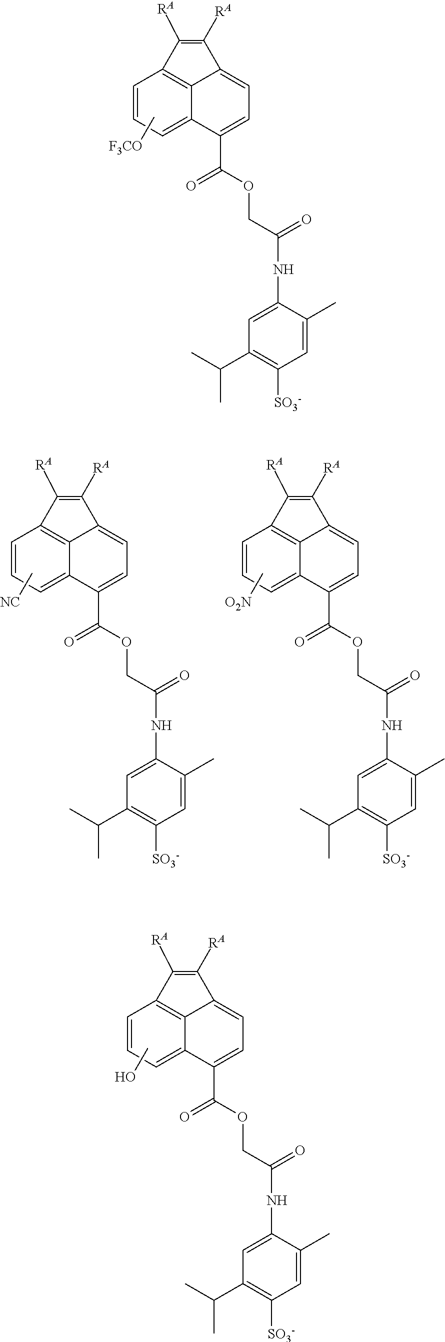

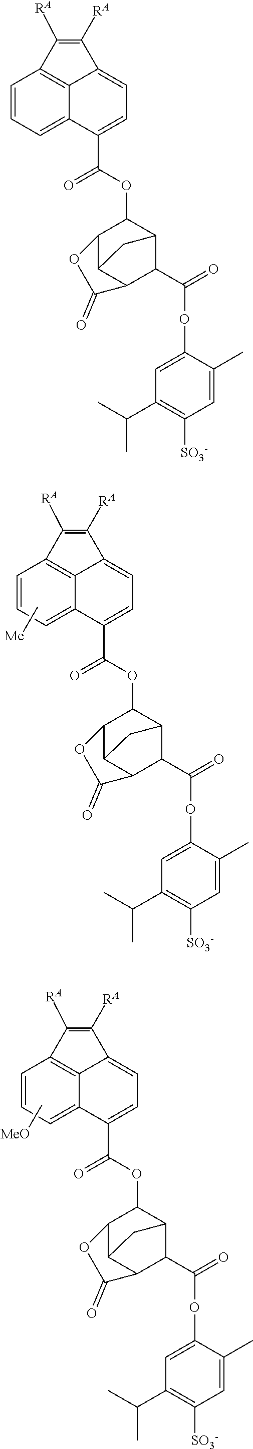

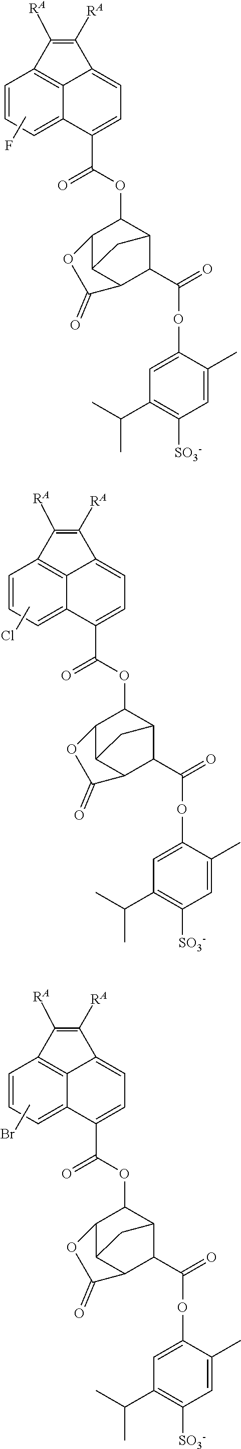

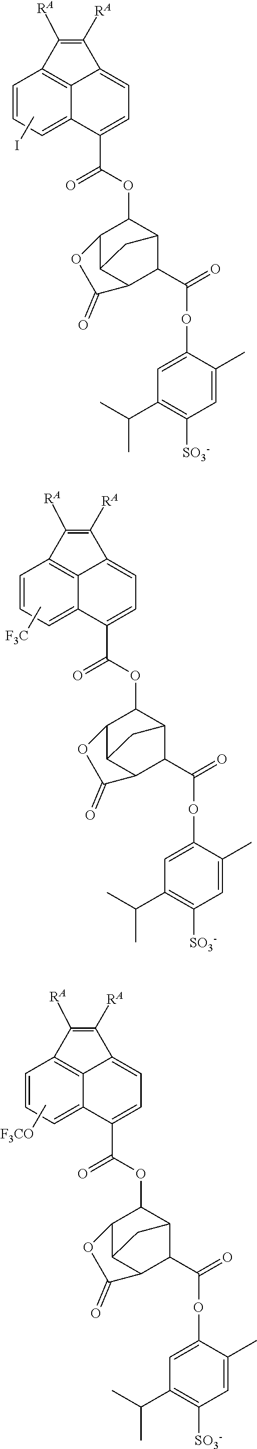

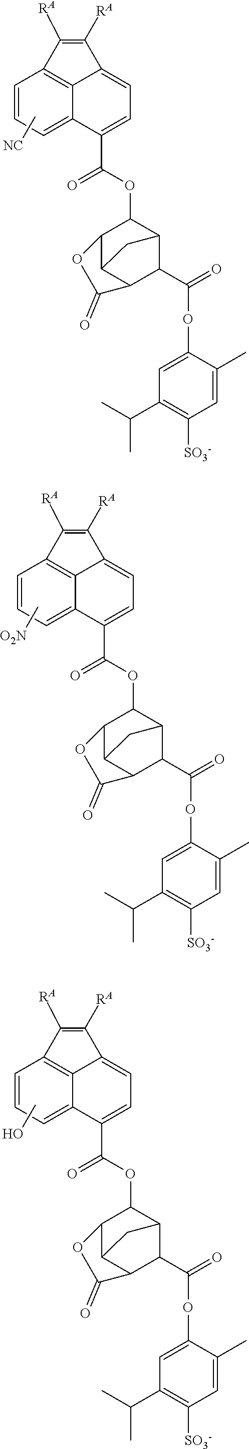

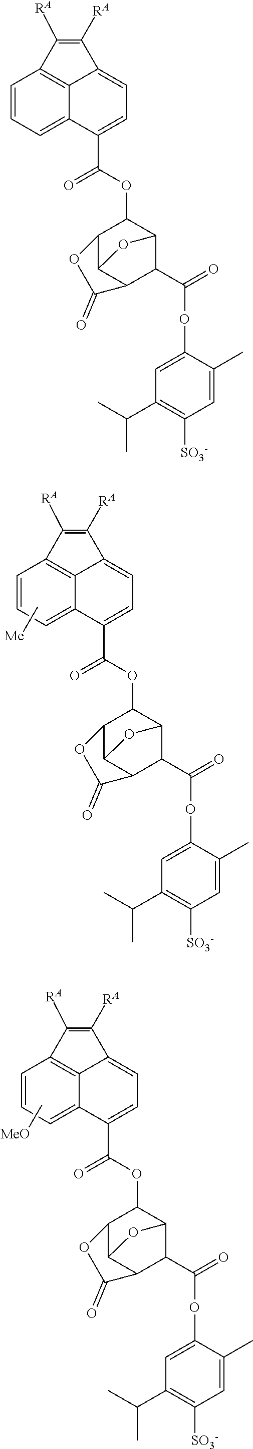

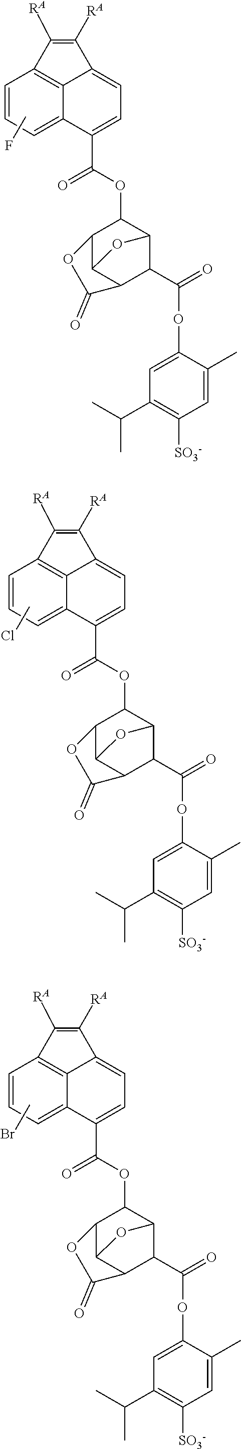

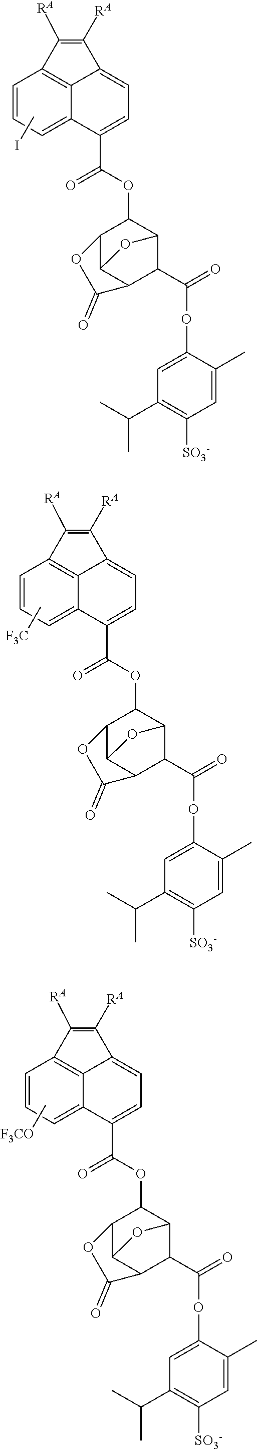

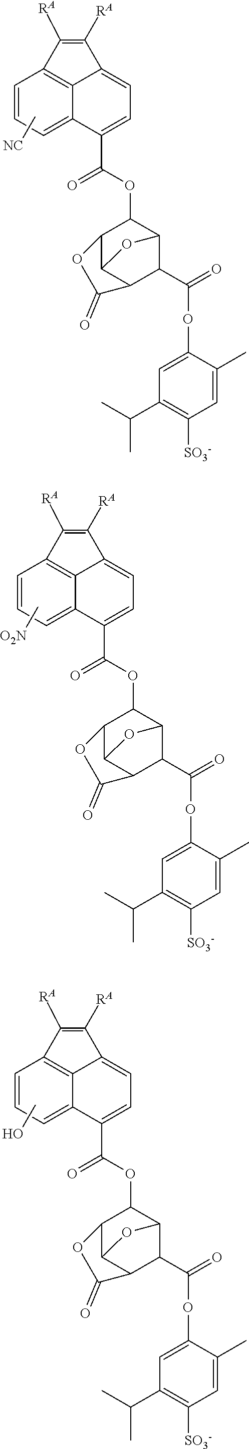

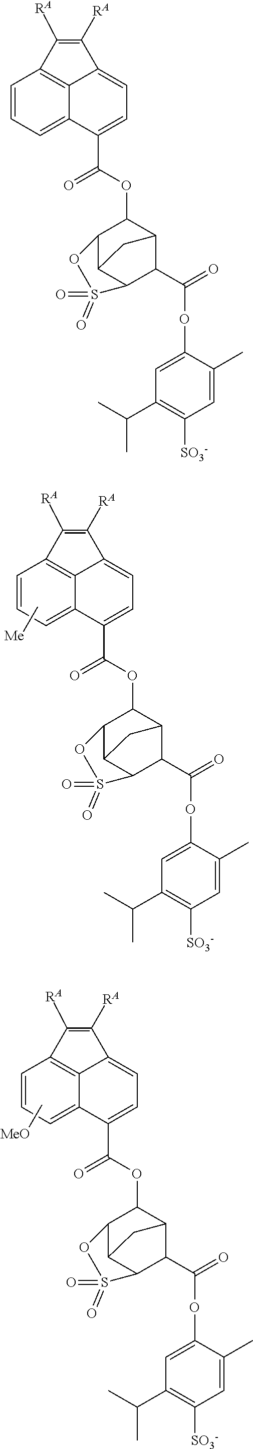

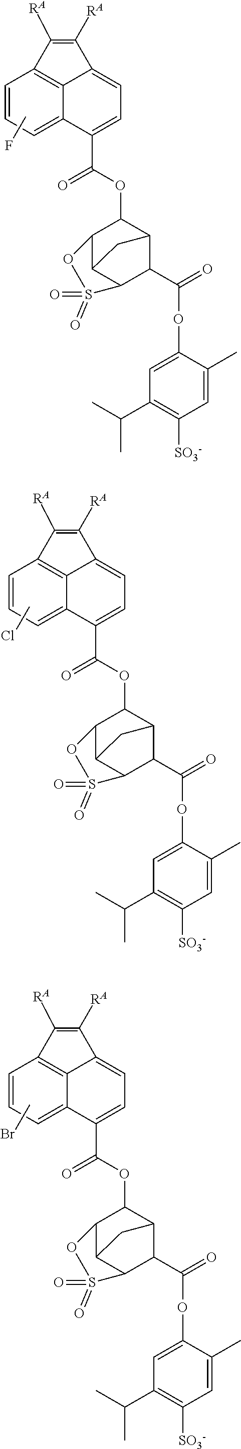

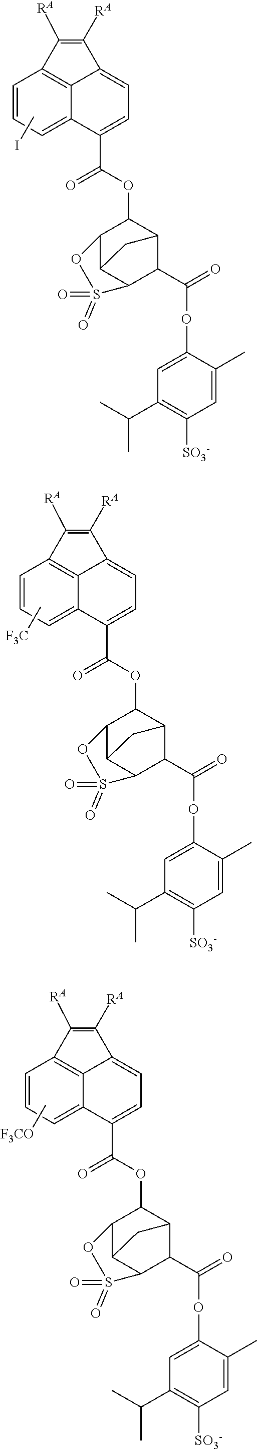

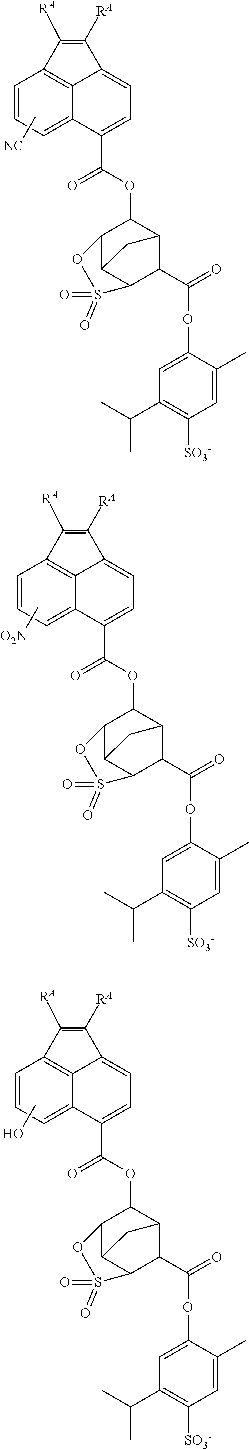

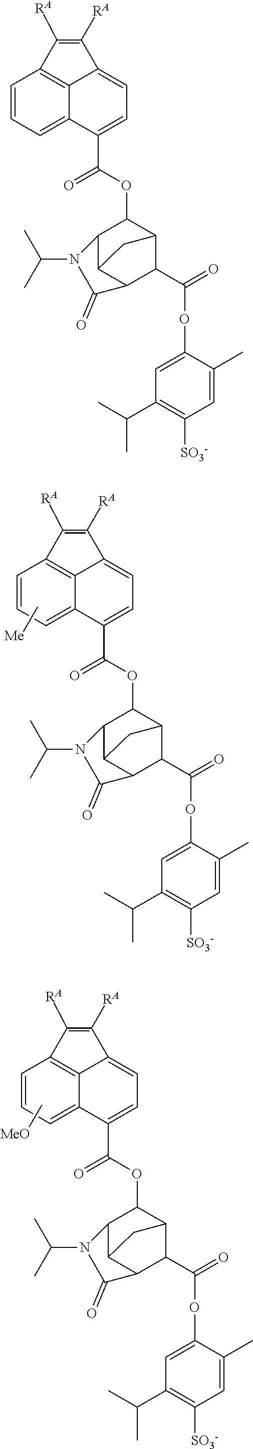

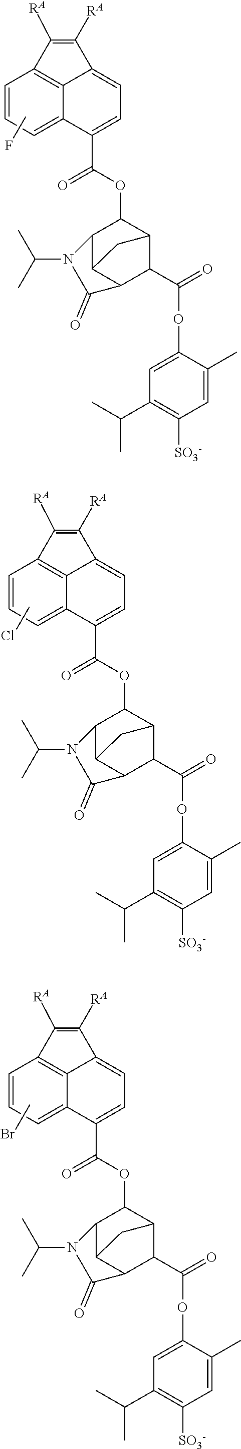

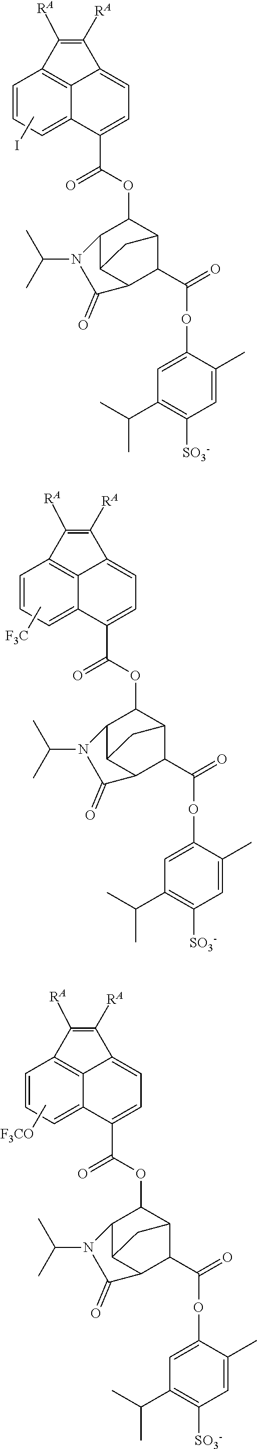

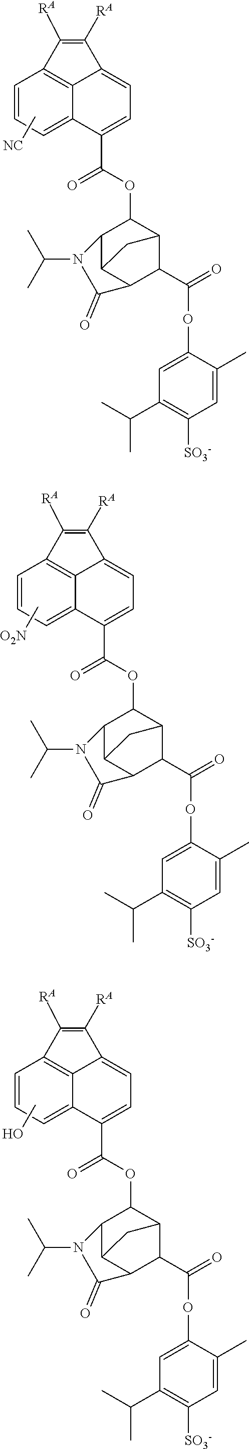

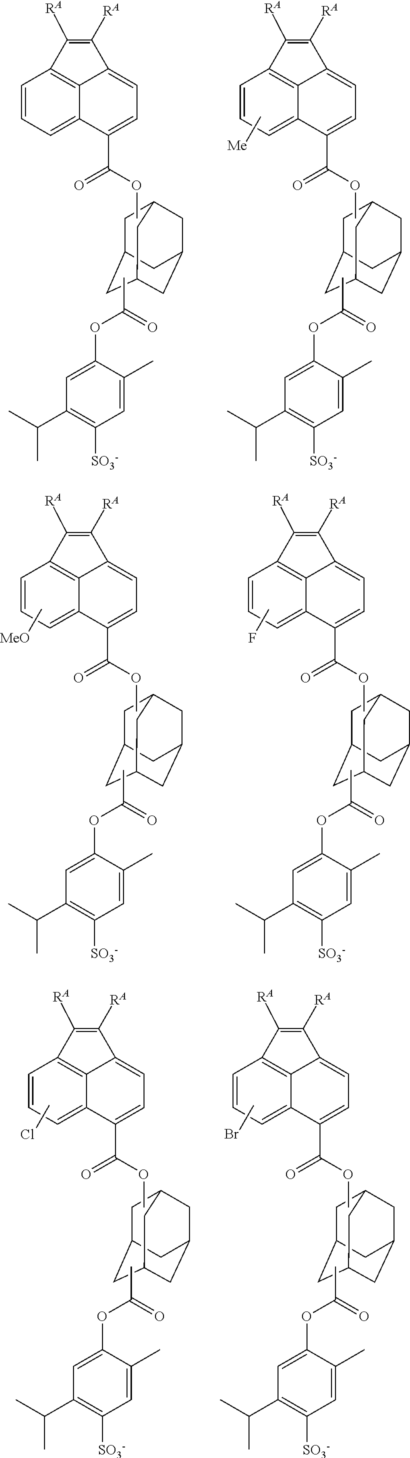

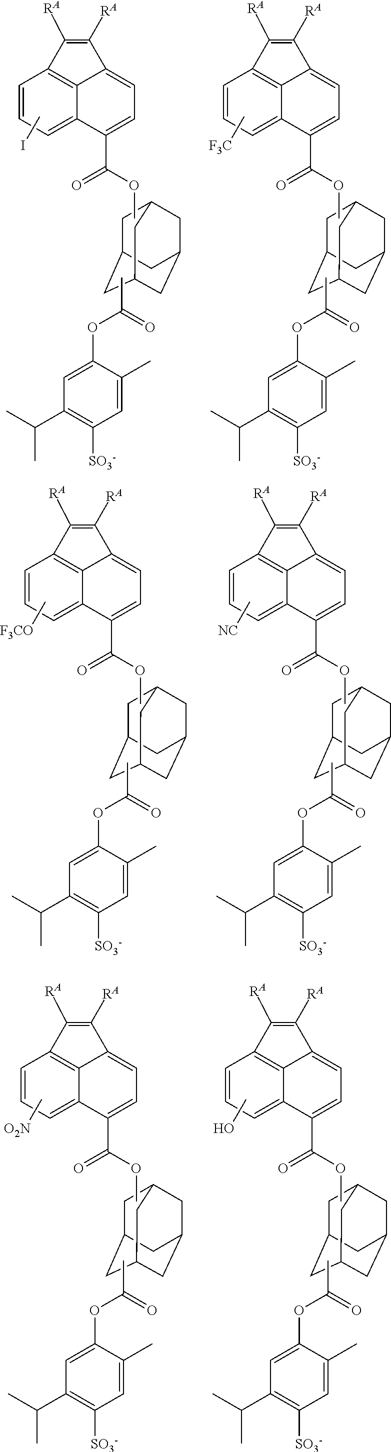

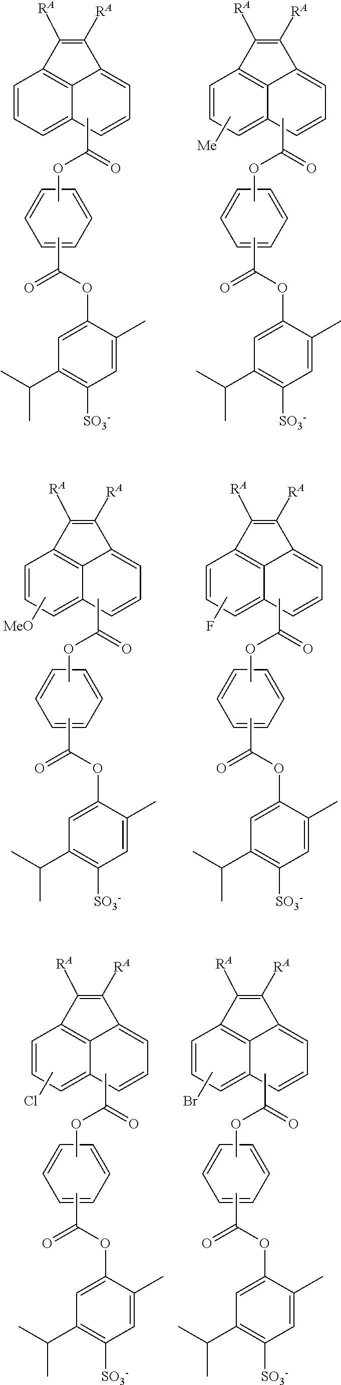

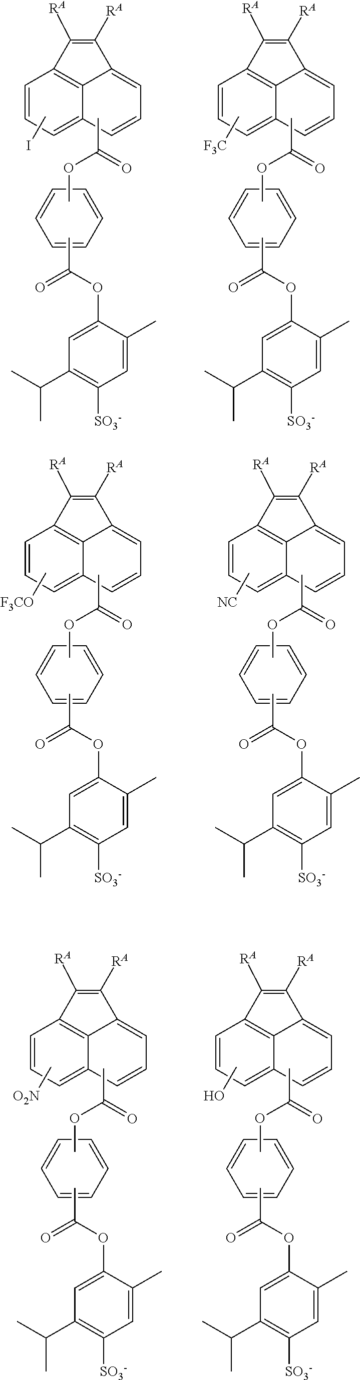

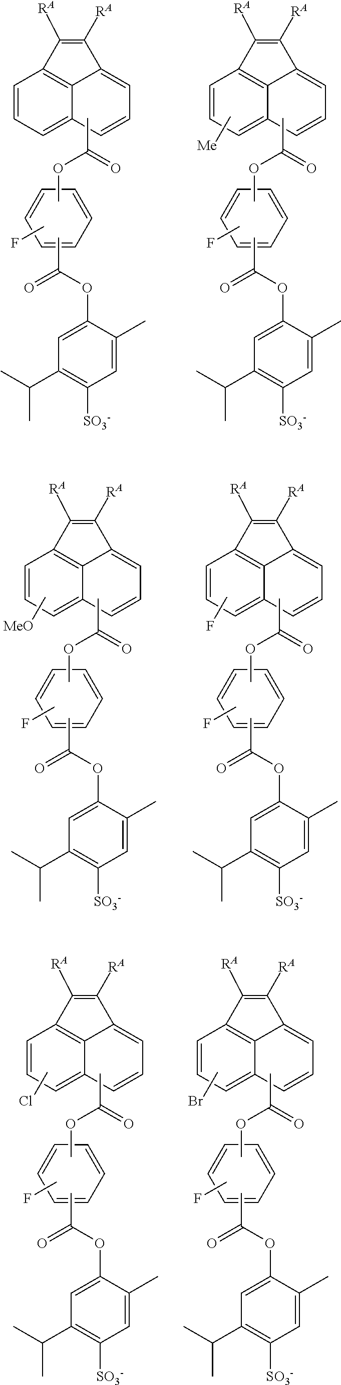

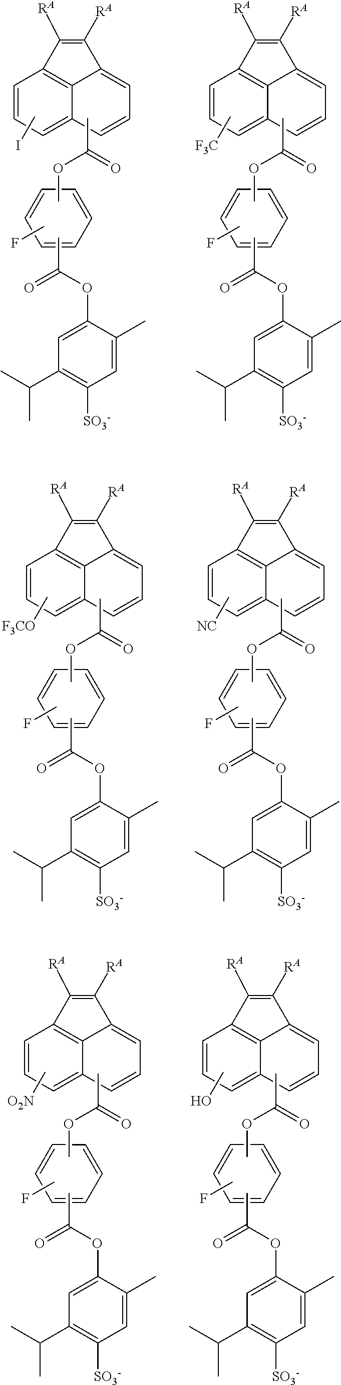

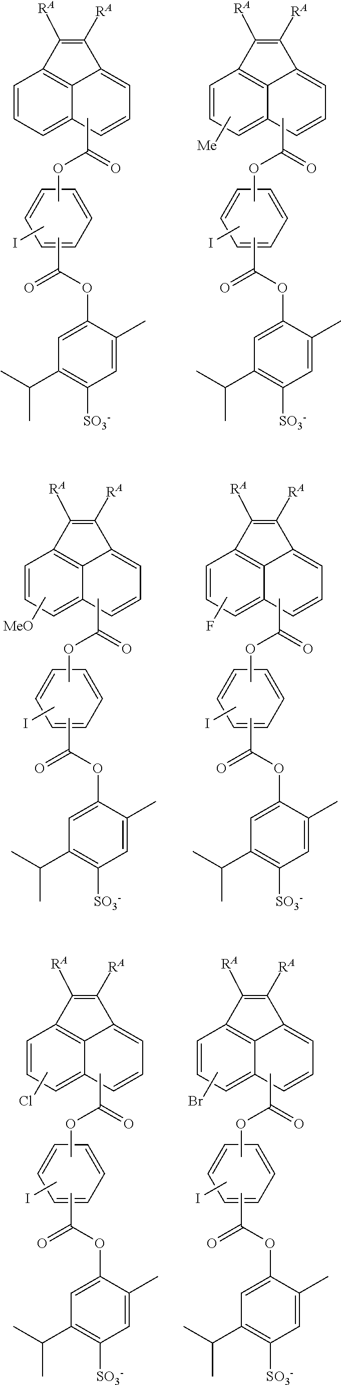

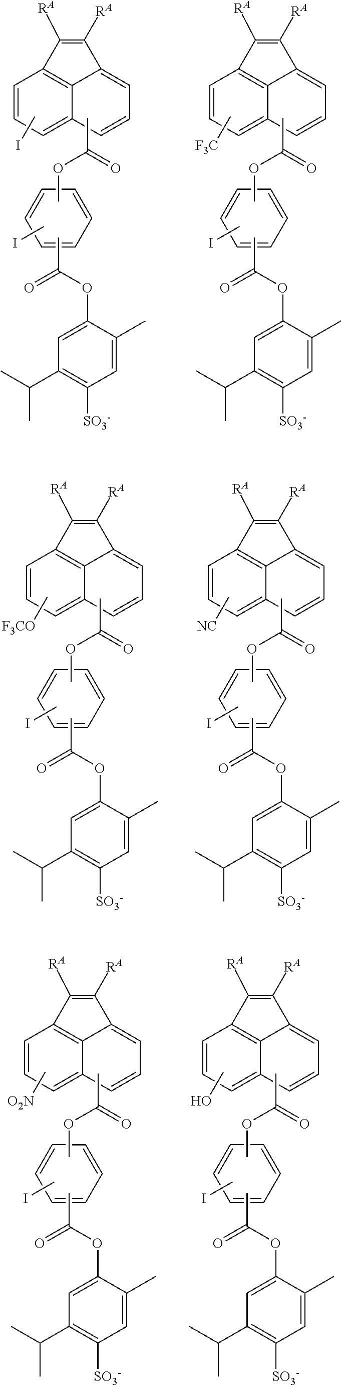

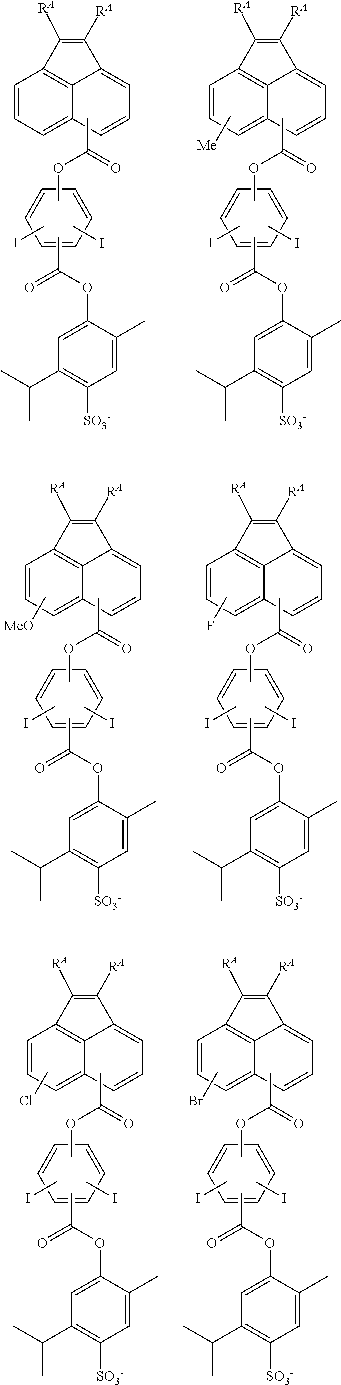

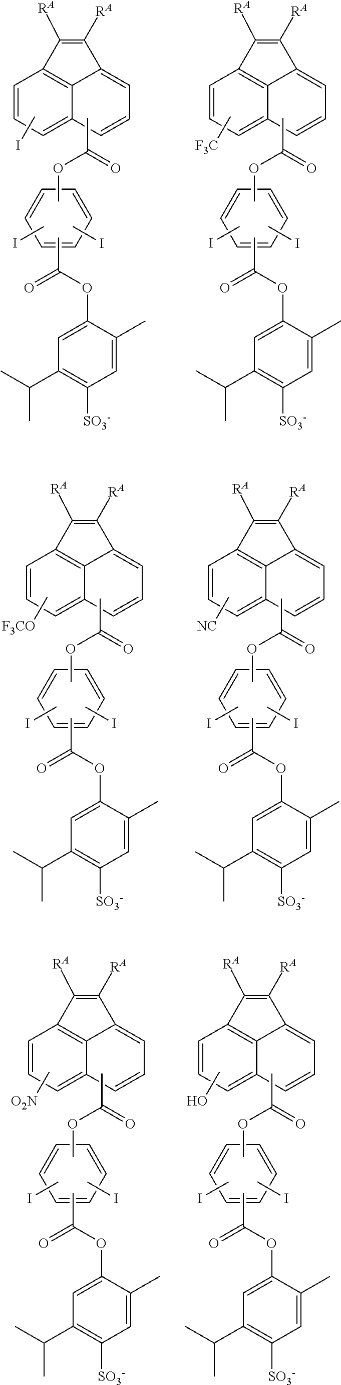

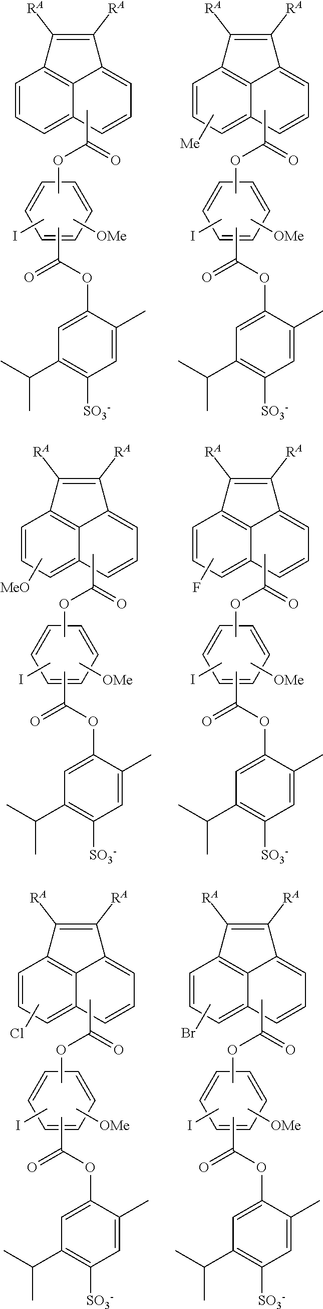

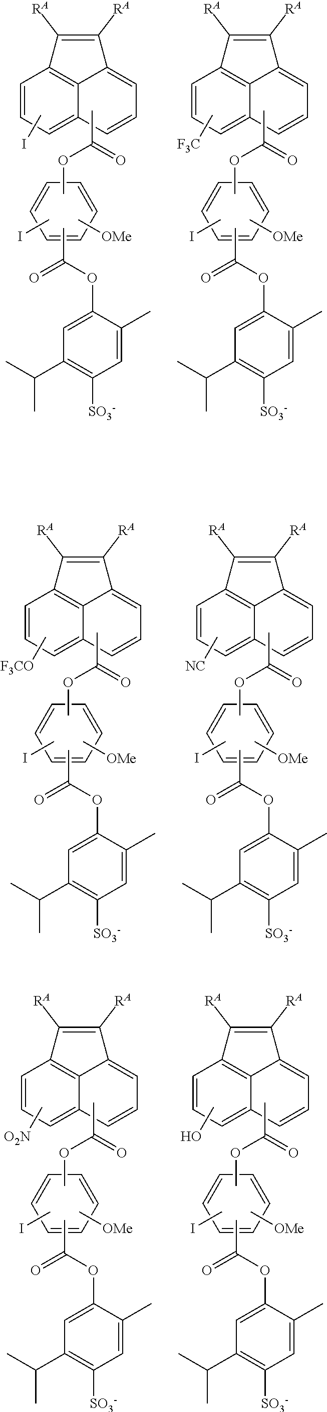

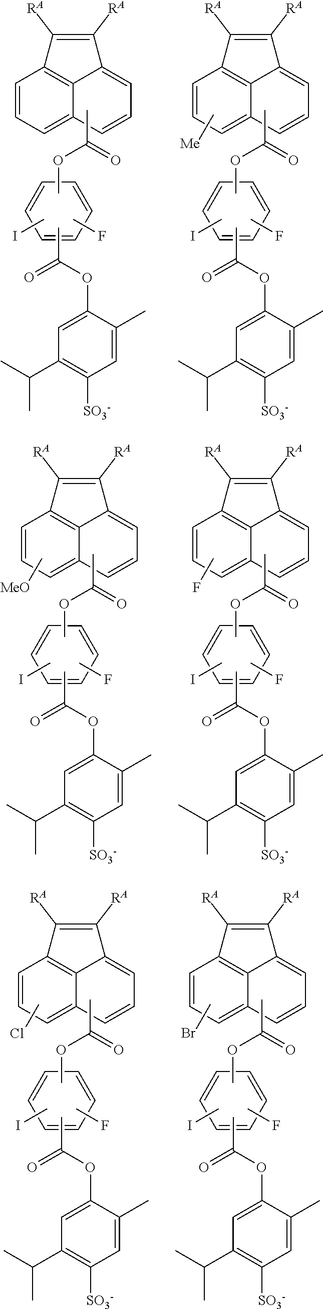

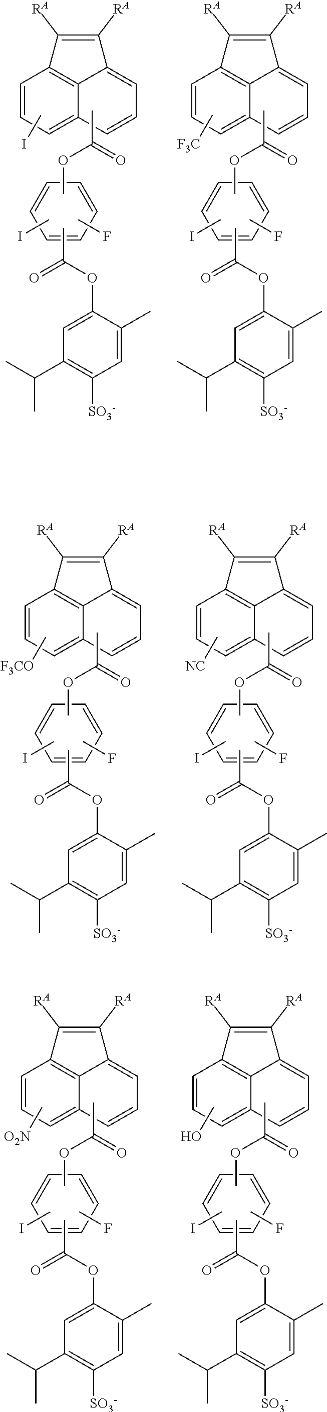

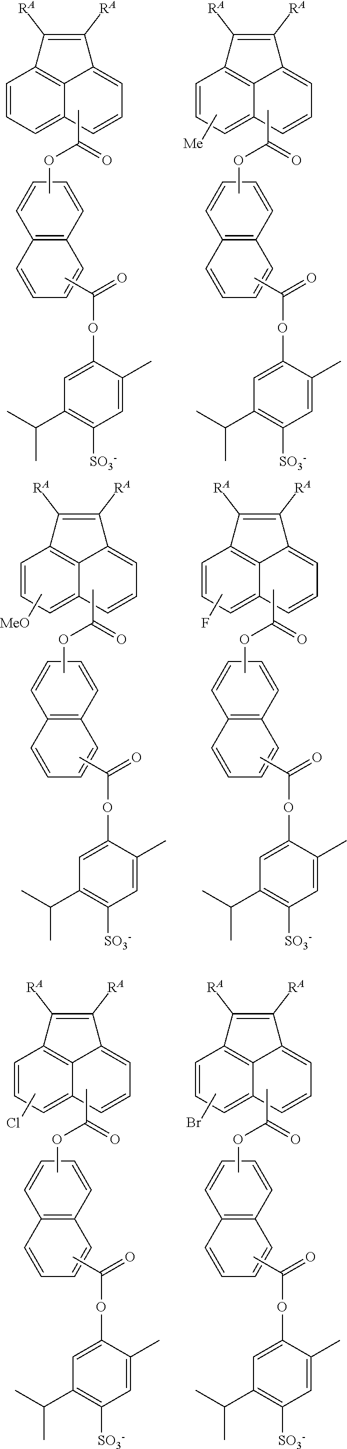

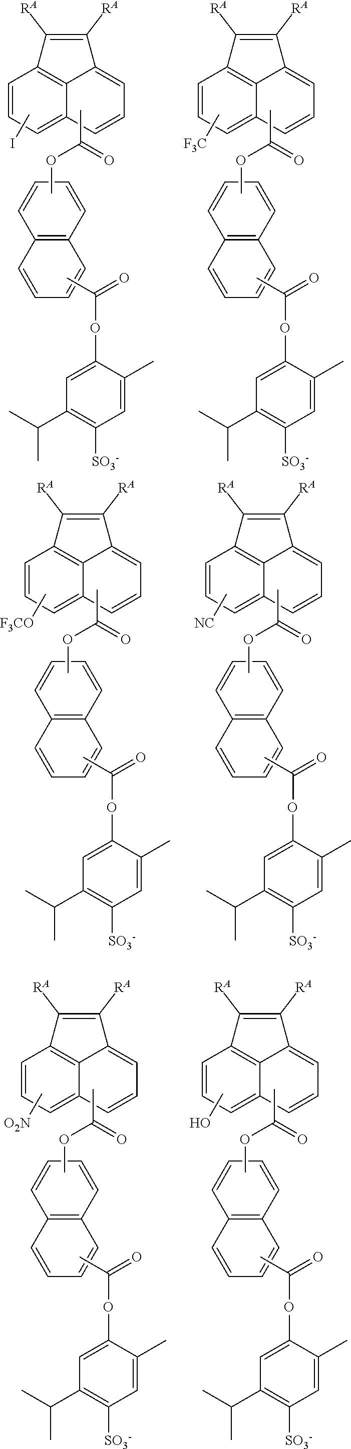

In formula (A), Z⁺ is an onium cation, preferably a sulfonium cation having the formula (Z-1) or iodonium cation having the formula (Z-2).

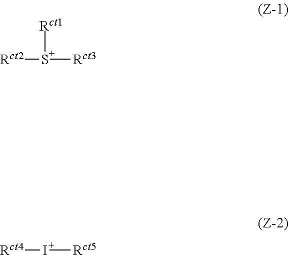

In formulae (Z-1) and (Z-2), R^{ct1} to R^{ct5} are each independently halogen or a C₁-C₃₀ hydrocarbyl group which may contain a heteroatom.

Examples of the halogen represented by R^{ct1} to R^{ct5} include fluorine, chlorine, bromine and iodine.

The hydrocarbyl group represented by R^{ct1} to R^{ct5} may be saturated or unsaturated and straight, branched or cyclic. Examples thereof include C₁-C₃₀ alkyl groups such as methyl, ethyl, n-propyl, isopropyl, n-butyl, isobutyl, sec-butyl, tert-butyl: C₃-C₃₀ cyclic saturated hydrocarbyl groups such as cyclopropyl, cyclopentyl, cyclohexyl, cyclopropylmethyl, 4-methylcyclohexyl, cyclohexylmethyl, norbornyl, adamantyl; C₂-C₃₀ alkenyl groups such as vinyl, 1-propenyl, 2-propenyl, butenyl, hexenyl: C₃-C₃₀ cyclic unsaturated hydrocarbyl groups such as cyclohexenyl: C₆-C₃₀ aryl groups such as phenyl, naphthyl, thienyl: C₇-C₃₀ aralkyl groups such as benzyl, 1-phenylethyl, 2-phenylethyl, and combinations thereof. Inter alia, aryl groups are preferred. In the hydrocarbyl groups, some or all hydrogen may be substituted by a moiety containing a heteroatom such as oxygen, sulfur, nitrogen or halogen, and some —CH₂— may be replaced by a moiety containing a heteroatom such as oxygen, sulfur or nitrogen, so that the group may contain a hydroxy, fluorine, chlorine, bromine, iodine, cyano, nitro, carbonyl, ether bond, ester bond, sulfonate ester bond, carbonate bond, lactone ring, sultone ring, carboxylic anhydride (—C(═O)—O—C(═O)—) or haloalkyl moiety.

Also, R^{ct1} and R^{ct2} may bond together to form a ring with the sulfur atom to which they are attached. Exemplary structures of the ring are shown below.

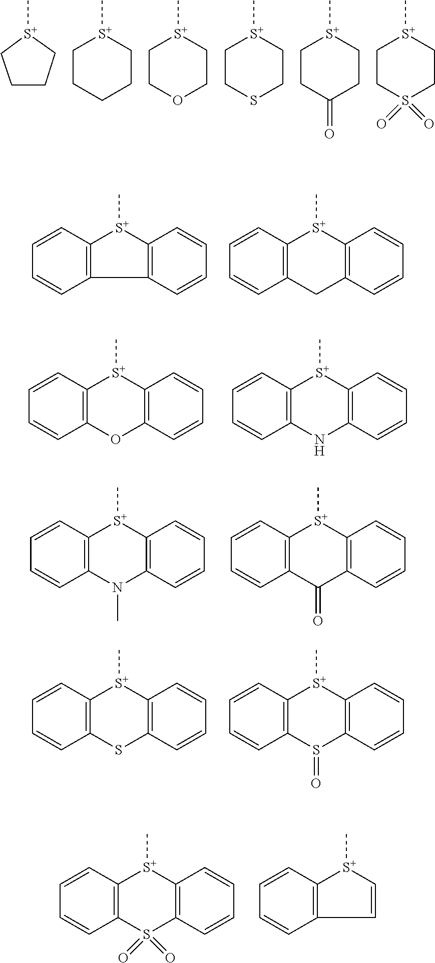

The broken line designates a point of attachment to R^{ct3}.

Examples of the sulfonium cation having formula (Z-1) include the cations shown in JP-A 2024-003744, paragraphs [0102]-[0125] and JP-A 2023-169812, paragraphs [0070]-[0085], but are not limited thereto.

Examples of the iodonium cation having formula (Z-2) include the cations shown in JP-A 2024-000259, paragraph [0181], but are not limited thereto.

A sulfonium cation having the formula (Z-3) is also preferable as the onium cation Z⁺.

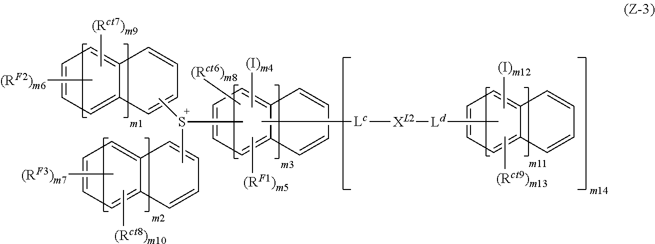

In formula (Z-3), m1 is 0 or 1. The relevant structure is a benzene ring when m1=0, and a naphthalene ring when m1=1. The benzene ring corresponding to m1-0 is preferred from the aspect of solvent solubility. The subscript m2 is 0 or 1. The relevant structure is a benzene ring when m2-0, and a naphthalene ring when m2=1. The benzene ring corresponding to m2=0 is preferred from the aspect of solvent solubility. The subscript m3 is 0 or 1. The relevant structure is a benzene ring when m3-0, and a naphthalene ring when m3=1. The benzene ring corresponding to m3=0 is preferred from the aspect of solvent solubility.

In formula (Z-3), m4 is 0, 1, 2, 3 or 4. As the number of iodine atoms in the cation structure increases, the compound becomes more absorptive to EUV, but so poor in solvent solubility that it may precipitate in a resist composition. For this reason, m4 is preferably 0, 1, 2 or 3, more preferably 0, 1 or 2.

In formula (Z-3), m5 is 0, 1, 2, 3 or 4. From the aspect of reactant availability, m5 is preferably 0, 1, 2 or 3, more preferably 0, 1 or 2. The subscript m6 is 0, 1, 2, 3, 4, 5 or 6. From the aspect of reactant availability, m6 is preferably 0, 1, 2 or 3, more preferably 0, 1 or 2. The subscript m7 is 0, 1, 2, 3, 4, 5 or 6. From the aspect of reactant availability, m7 is preferably 0, 1, 2 or 3, more preferably 0, 1 or 2.

In formula (Z-3), m8 is 0, 1 or 2. From the aspect of reactant availability, m8 is preferably 0 or 1. The subscript m9 is 0, 1 or 2. From the aspect of reactant availability, m9 is preferably 0 or 1. The subscript m10 is 0, 1 or 2. From the aspect of reactant availability, m10 is preferably 0 or 1.

In formula (Z-3), m11 is 0 or 1. The relevant structure is a benzene ring when m11-0, and a naphthalene ring when m11=1. The benzene ring corresponding to m11=0 is preferred from the aspect of solvent solubility.

In formula (Z-3), m12 is 0, 1, 2, 3 or 4. As the number of iodine atoms in the cation structure increases, the compound becomes more absorptive to EUV, but so poor in solvent solubility that it may precipitate in a resist composition. For this reason, m12 is preferably 0, 1, 2 or 3, more preferably 0, 1 or 2.

In formula (Z-3), m13 is 0, 1 or 2. From the aspect of reactant availability, m13 is preferably 0 or 1. The subscript m14 is 0, 1 or 2. From the aspect of synthesis, m14 is preferably 0 or 1.

The subscripts m1 to m14 are in the range: 0≤m6+m9≤4 when m1=0, 0≤m6+m9≤6 when m1=1; 0≤m7+m10≤4 when m2=0, 0≤m7+m10≤6 when m2=1; 1≤m4+m5+m8+m14≤4 when m3=0, 1≤m4+m5+m8+m14≤6 when m3=1; 0≤m12+m13≤4 when m11=0, 0≤m12+m13≤6 when m11=1; and m4+m12≥1.

In formula (Z-3), R^{F1} to R^{F3} are each independently fluorine, a C₁-C₆ fluorinated saturated hydrocarbyl group, C₁-C₆ fluorinated saturated hydrocarbyloxy group, or C₁-C₆ fluorinated saturated hydrocarbylthio group. Of these, trifluoromethyl, trifluoromethoxy, and trifluorothiomethoxy are preferred. A plurality of R^{F1} may be identical or different when m5 is 2, 3 or 4, a plurality of R^{F2} may be identical or different when m6 is 2, 3, 4, 5 or 6, and a plurality of R^{F3} may be identical or different when m7 is 2, 3, 4, 5 or 6.

In formula (Z-3), R^{ct6} to R^{ct9} are each independently halogen exclusive of iodine and fluorine, nitro, cyano, a C₁-C₂₀ hydrocarbyl group which may contain a heteroatom, C₁-C₂₀ hydrocarbyloxy group which may contain a heteroatom, or C₁-C₂₀ hydrocarbylthio group which may contain a heteroatom. The hydrocarbyl group and hydrocarbyl moiety in the hydrocarbyloxy and hydrocarbylthio groups may be saturated or unsaturated and straight, branched or cyclic. Examples thereof are as exemplified above for the hydrocarbyl group R¹ in formula (A). In the hydrocarbyl group and hydrocarbyl moiety in the hydrocarbyloxy and hydrocarbylthio groups, some or all of the hydrogen atoms may be substituted by a moiety containing a heteroatom such as oxygen, sulfur, nitrogen or halogen, and some constituent —CH₂— may be replaced by a moiety containing a heteroatom such as oxygen, sulfur or nitrogen, so that the group may contain a hydroxy moiety, cyano moiety, fluorine, chlorine, bromine, iodine, carbonyl moiety, ether bond, ester bond, sulfonate ester bond, carbonate bond, lactone ring, sultone ring, carboxylic anhydride (—C(═O)—O—C(═O)—) or haloalkyl moiety.

When m8=2, two R^{ct6} may be identical or different and two R^{ct6} may bond together to form a ring with the carbon atoms to which they are attached. When m9=2, two R^{ct7} may be identical or different and two R^{ct7} may bond together to form a ring with the carbon atoms to which they are attached. When m10=2, two R^{ct8} may be identical or different and two R^{ct8} may bond together to form a ring with the carbon atoms to which they are attached. When m13=2, two R^{ct9} may be identical or different and two R^{ct9} may bond together to form a ring with the carbon atoms to which they are attached. Examples of the ring thus formed include cyclopropane, cyclobutane, cyclopentane, cyclohexane, norbornane, and adamantane rings. In the ring, some or all of the hydrogen atoms may be substituted by a moiety containing a heteroatom such as oxygen, sulfur, nitrogen or halogen, and some constituent —CH₂— may be replaced by a moiety containing a heteroatom such as oxygen, sulfur or nitrogen, so that the ring may contain a hydroxy moiety, fluorine, chlorine, bromine, iodine, cyano moiety, carbonyl moiety, ether bond, ester bond, sulfonate ester bond, carbonate bond, lactone ring, sultone ring, carboxylic anhydride (—C(═O)—O—C(═O)—) or haloalkyl moiety.

The aromatic rings directly bonded to S⁺ in the sulfonium cation having formula (Z-3) may bond together to form a ring with S⁺. Exemplary structures of the ring are shown below.

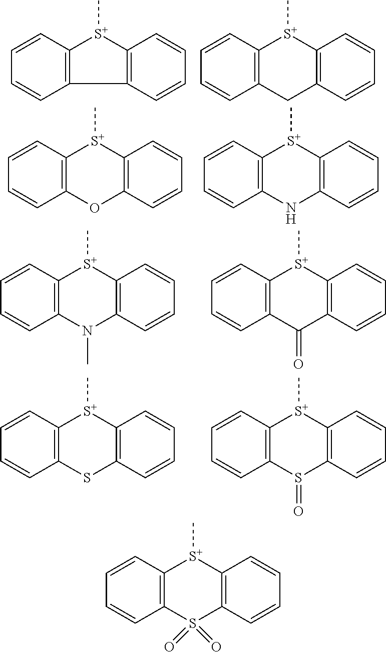

In formula (Z-3), L^{C} and L^{D} are each independently a single bond, ether bond, ester bond, sulfonate ester bond, amide bond, sulfonamide bond, carbonate bond or carbamate bond. L^{C} is preferably a single bond, ether bond, ester bond or sulfonate ester bond, more preferably an ester bond or sulfonate ester bond. L^{D} is preferably a single bond, ether bond or ester bond, more preferably a single bond.

In formula (Z-3), X^{L2} is a single bond or a C₁-C₄₀ hydrocarbylene group which may contain a heteroatom. Examples of the C₁-C₄₀ hydrocarbylene group which may contain a heteroatom are as exemplified above for the C₁-C₄₀ hydrocarbylene group which may contain a heteroatom, represented by X^{L1}, but not limited thereto.

Of the sulfonium cations having formula (Z-3), those having the formula (Z-3-1) are preferred.

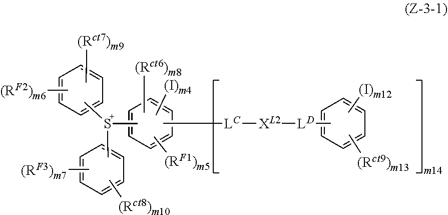

Herein m4 to m10, m12 to m14, R^{F1} to R^{F3}, R^{ct6} to R^{ct9}, L^{C}, L^{D}, and X¹² are as defined above.

Of the sulfonium cations having formula (Z-3-1), those having the formula (Z-3-2) are preferred.

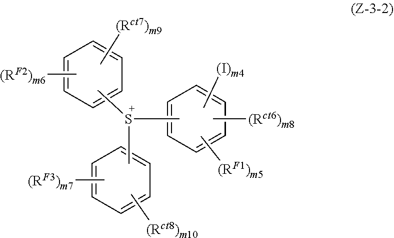

Herein m4 to m10, R^{F1} to R^{F3}, and R^{ct6} to R^{ct8} are as defined above.

Examples of the sulfonium cation having formula (Z-3) are shown below, but not limited thereto.

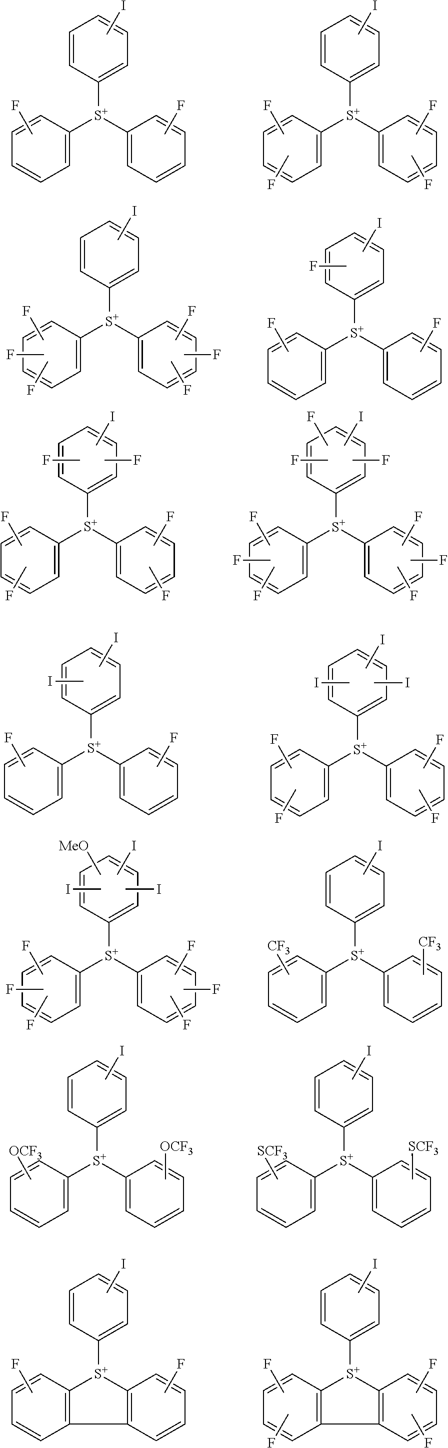

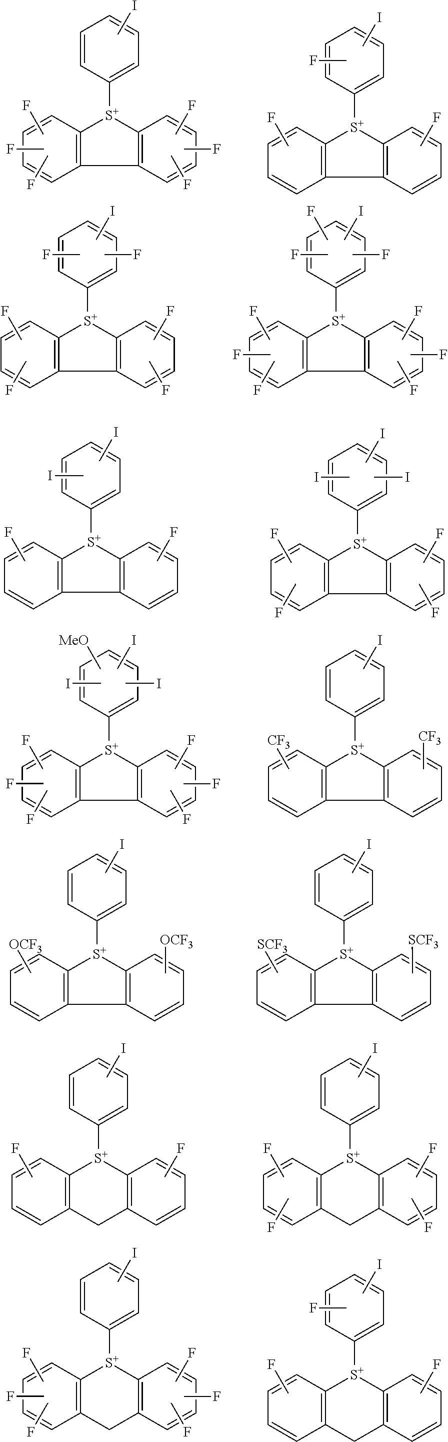

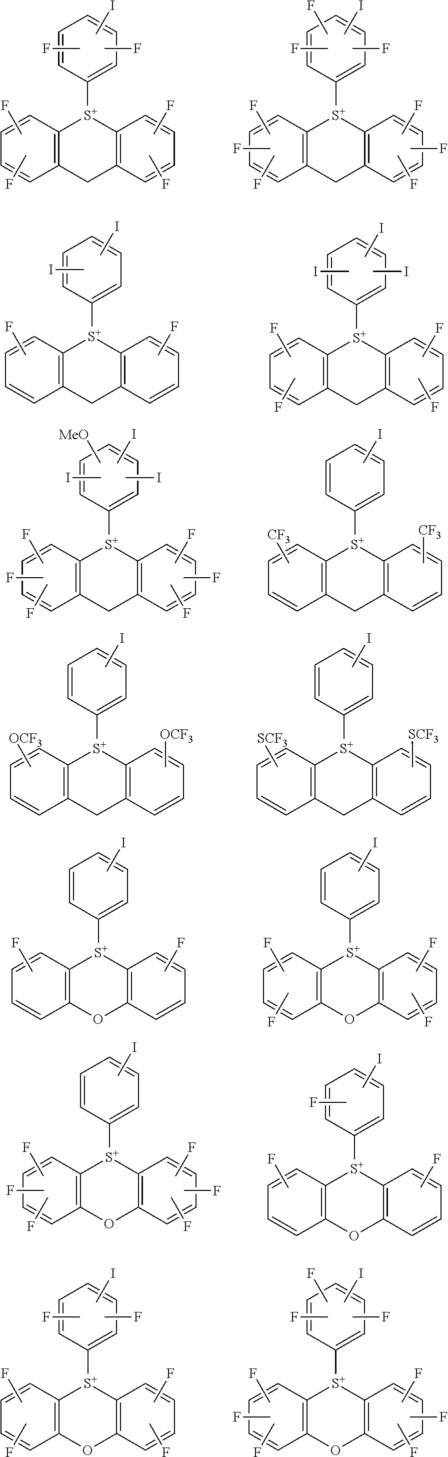

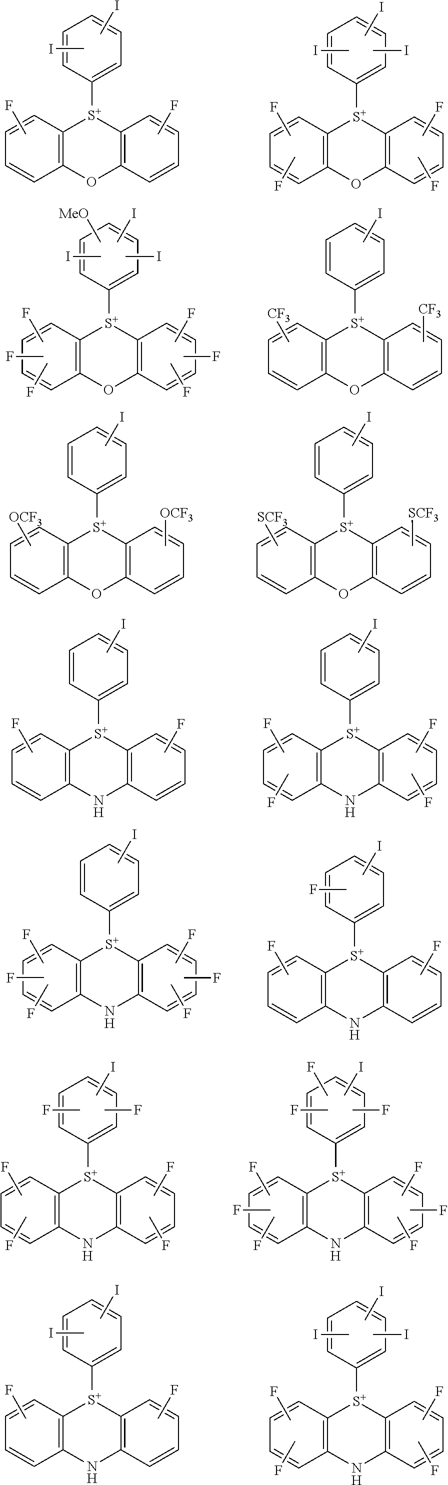

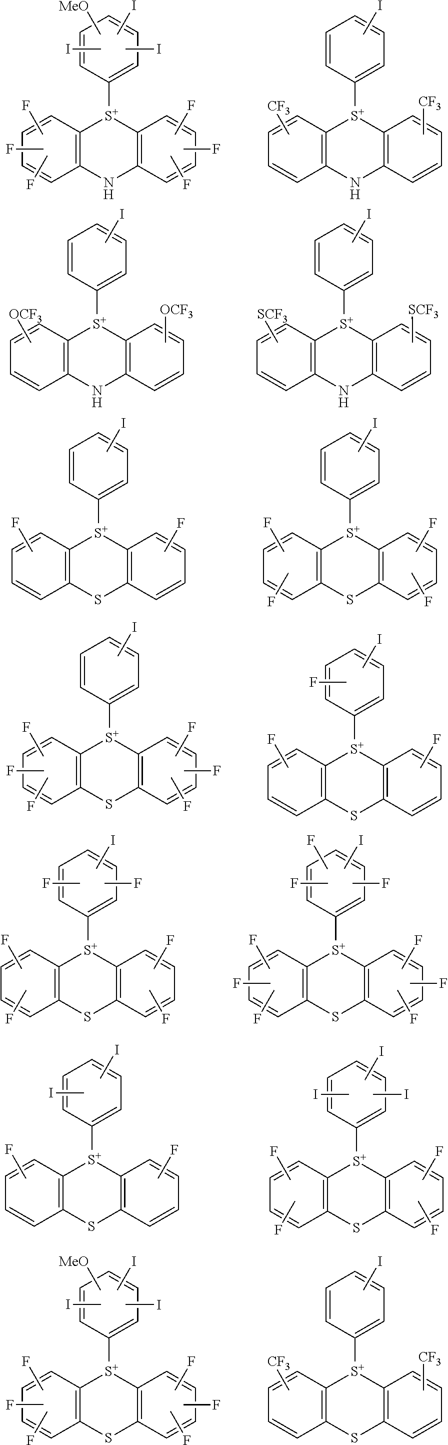

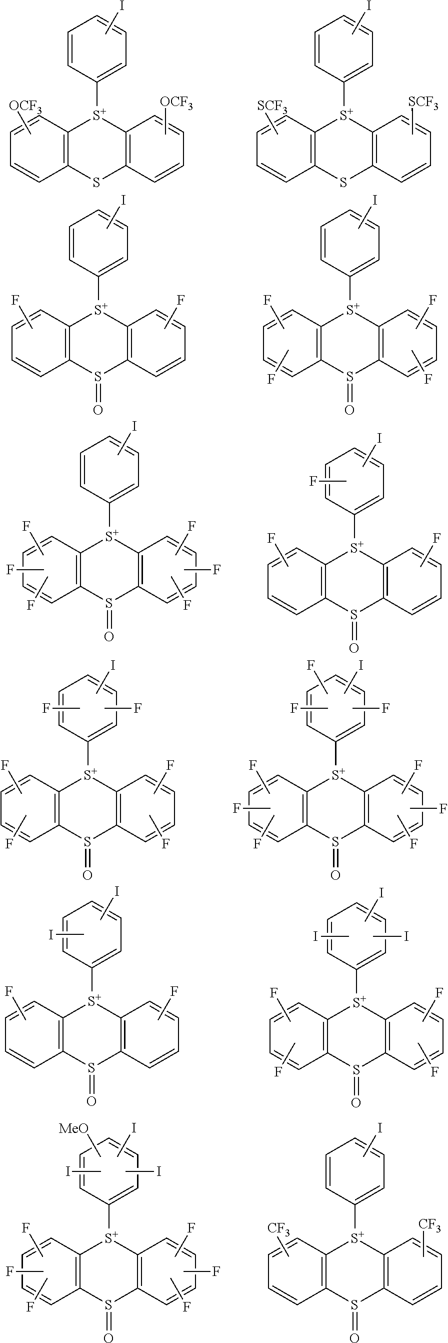

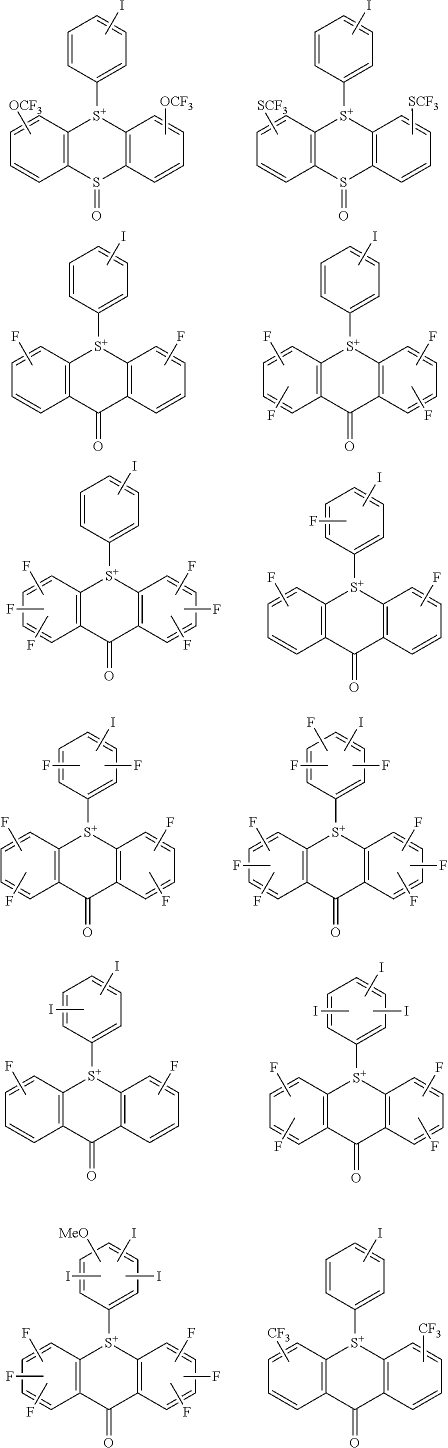

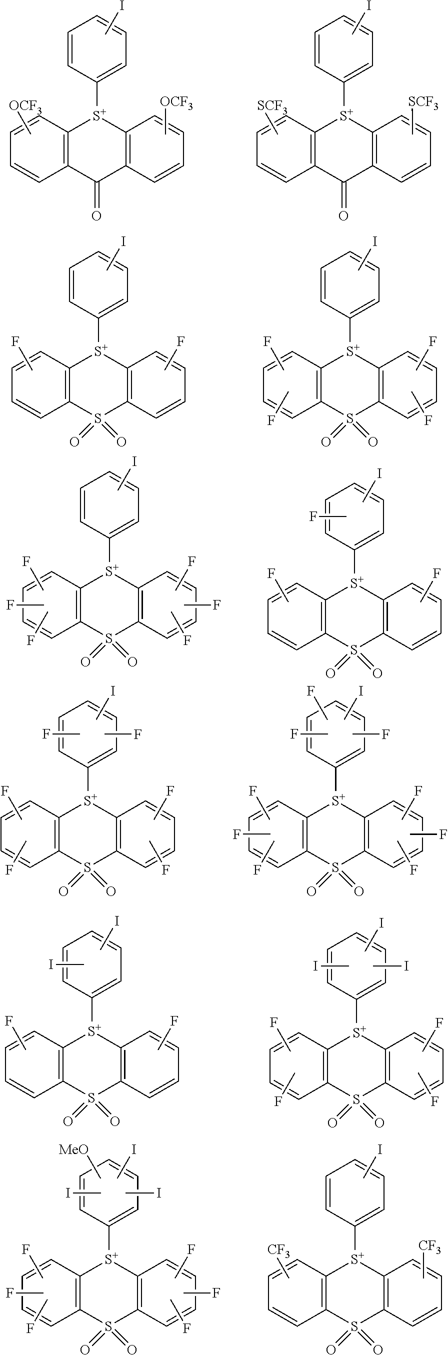

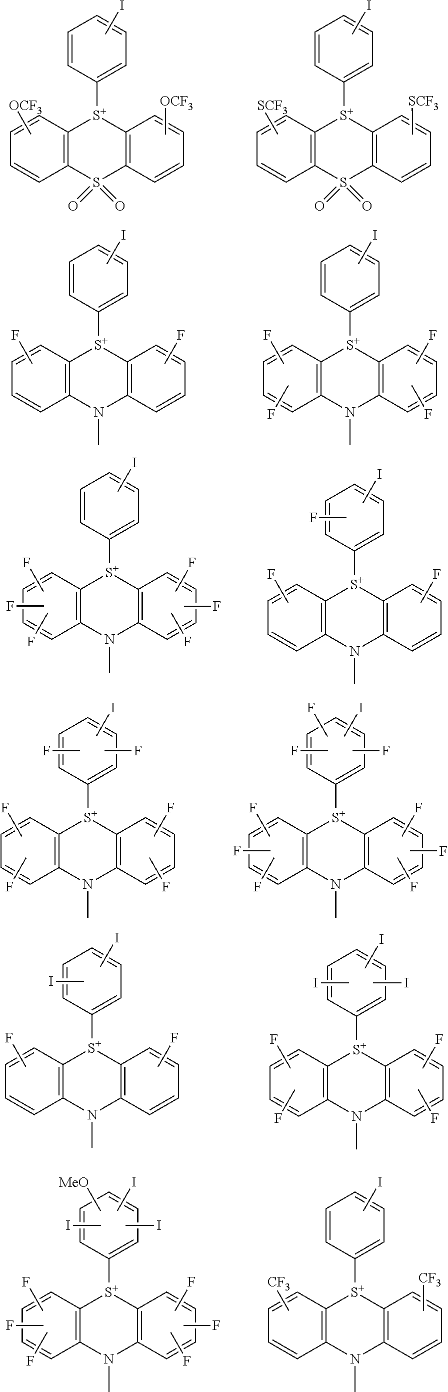

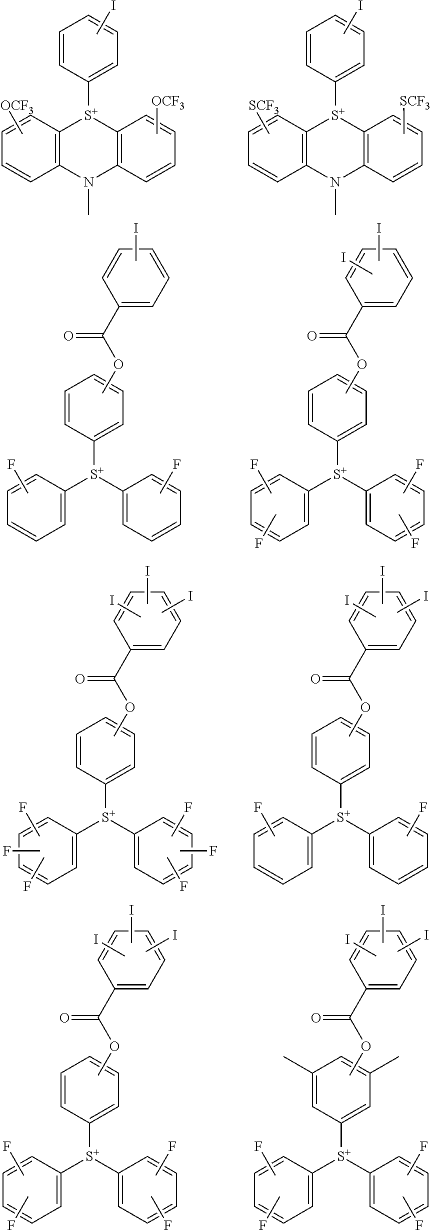

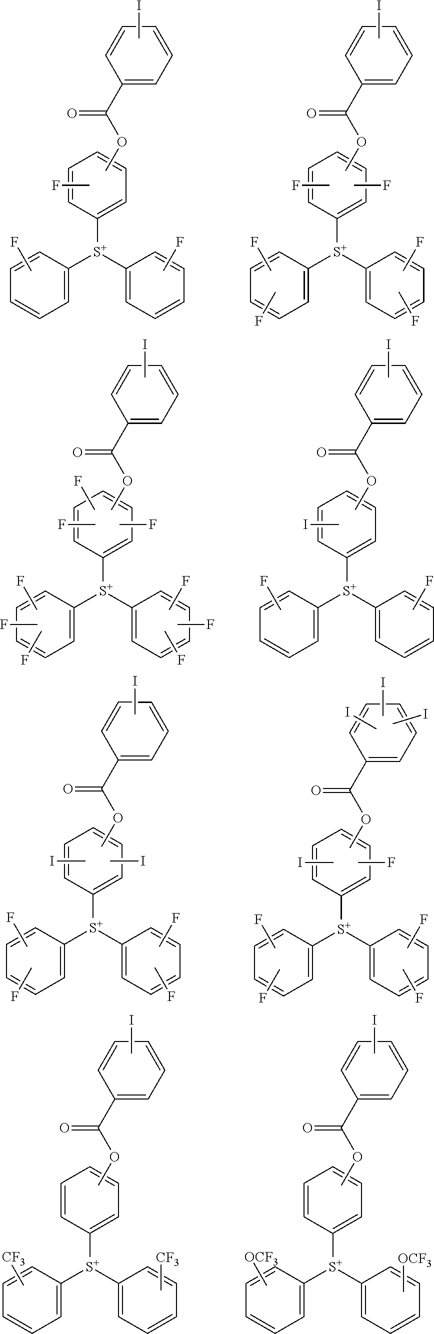

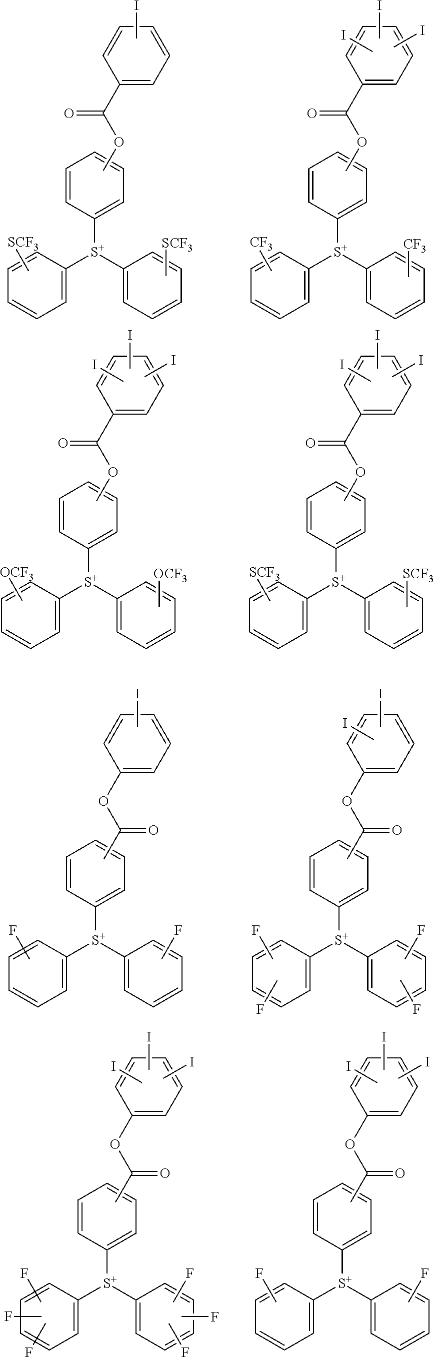

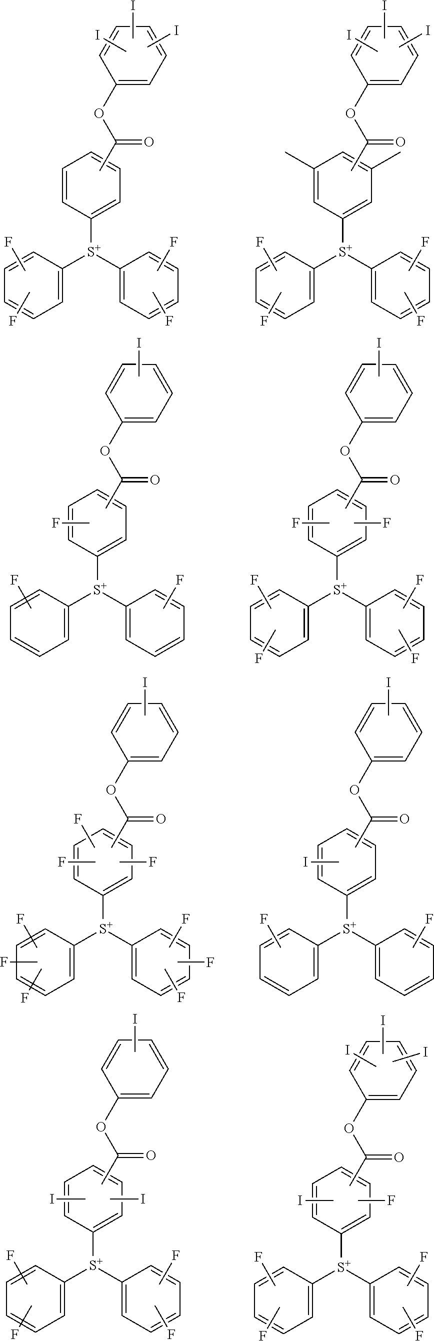

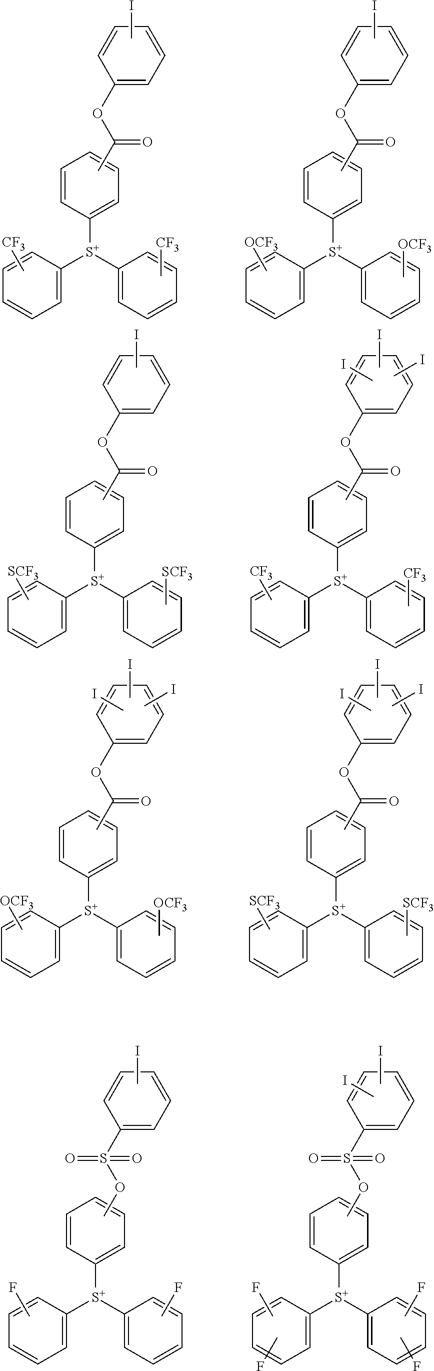

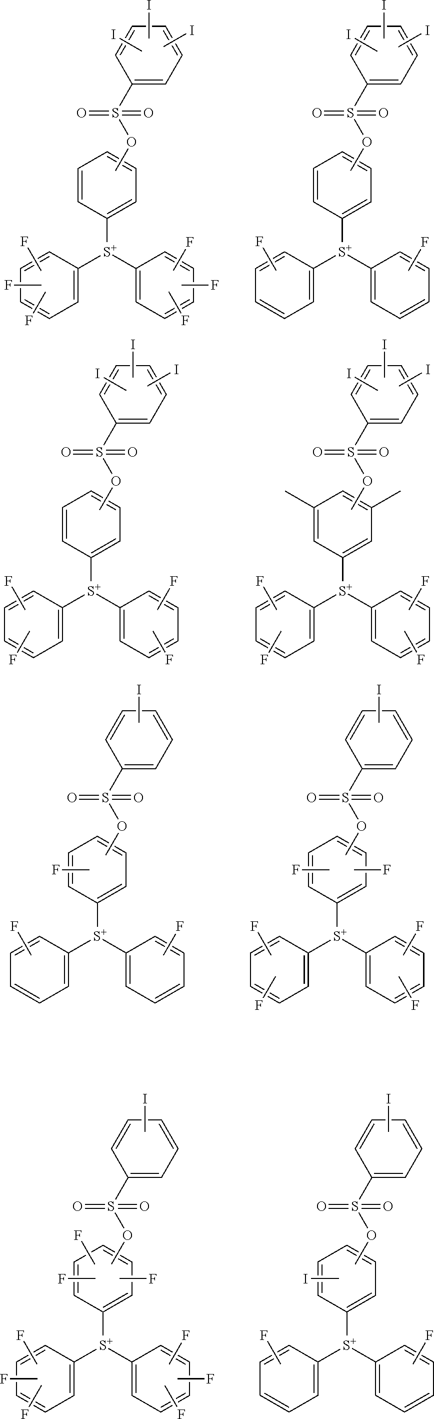

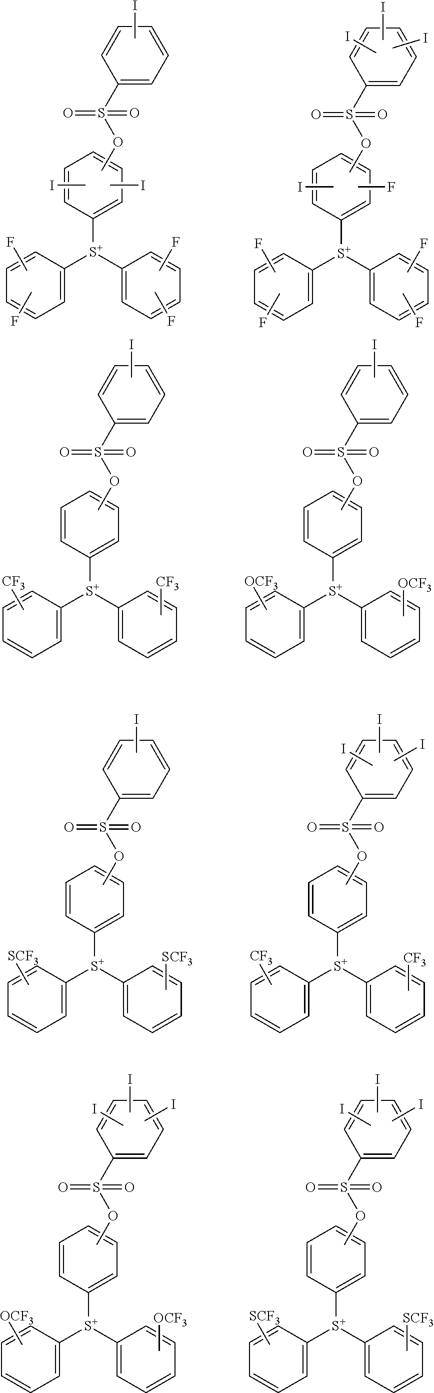

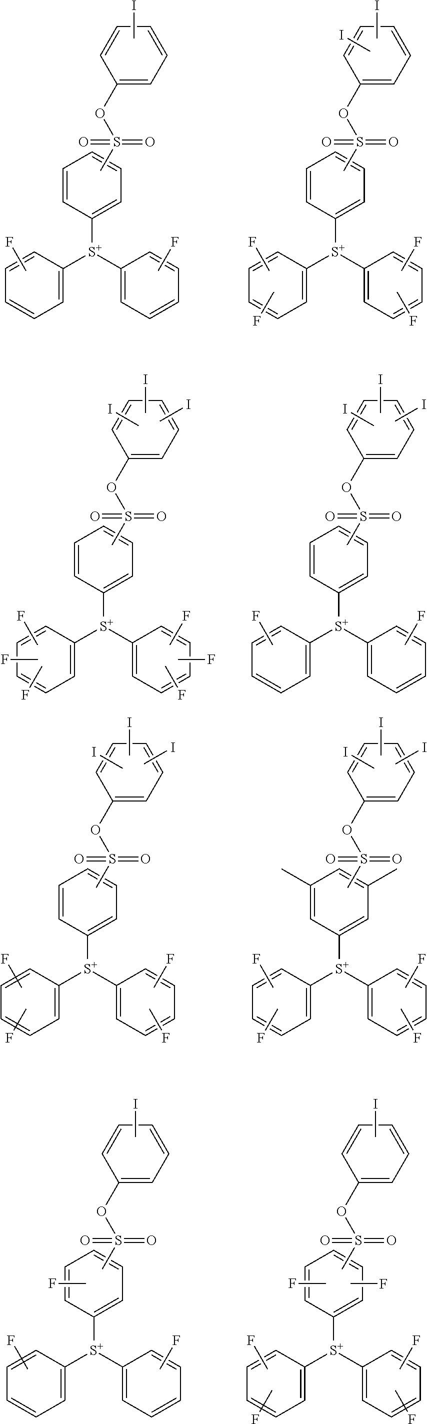

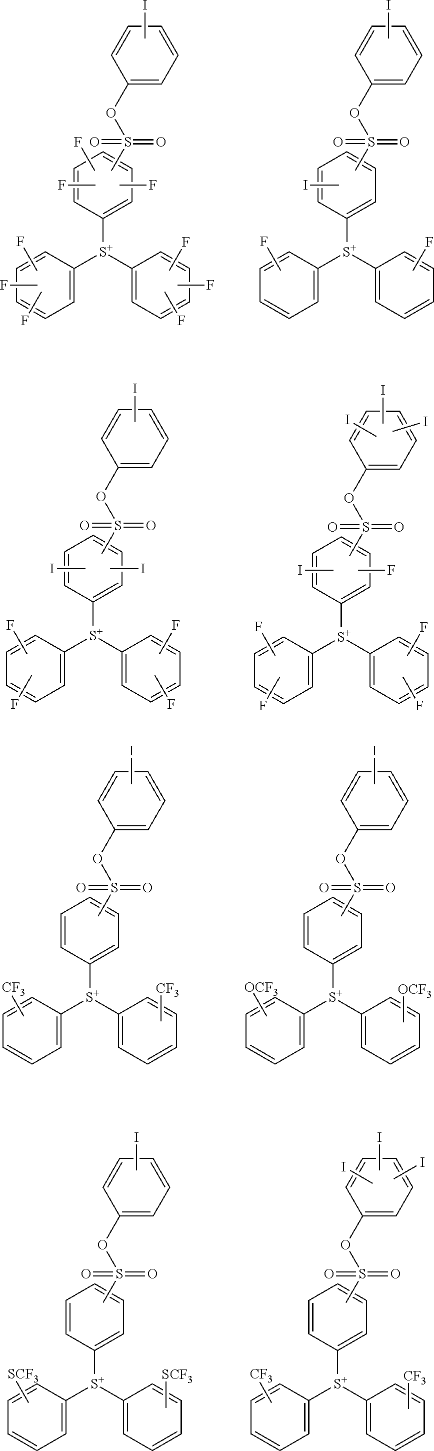

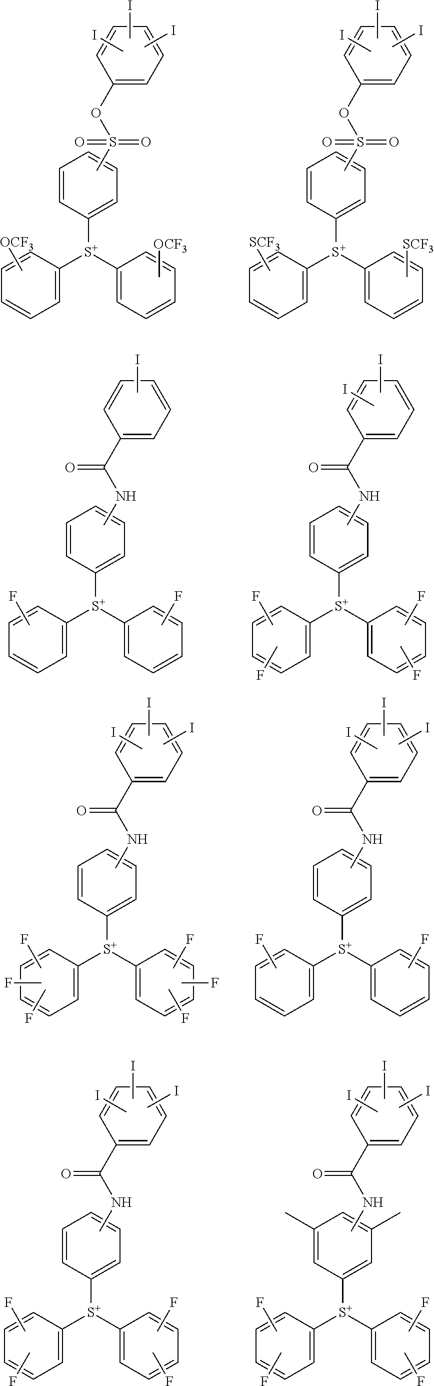

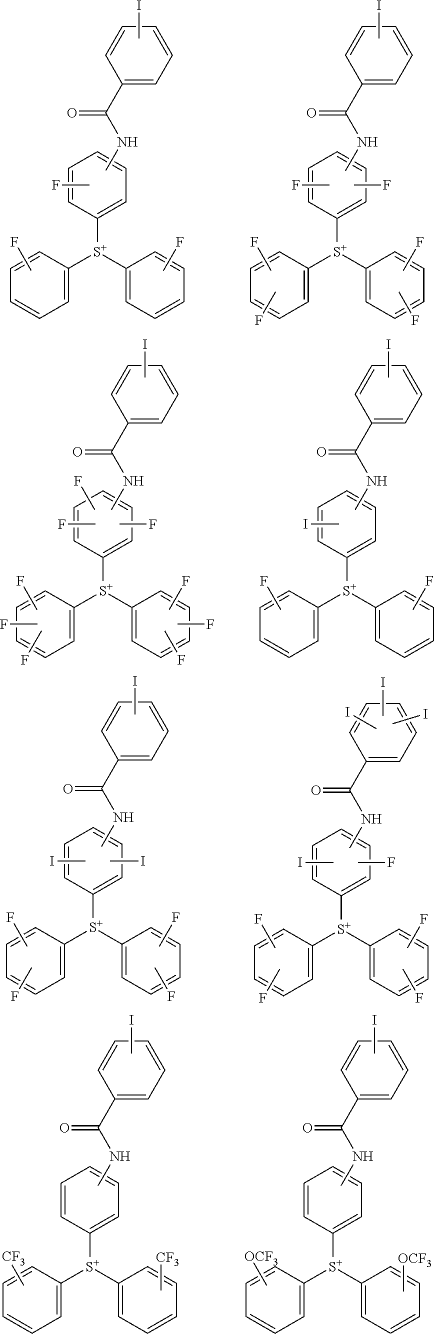

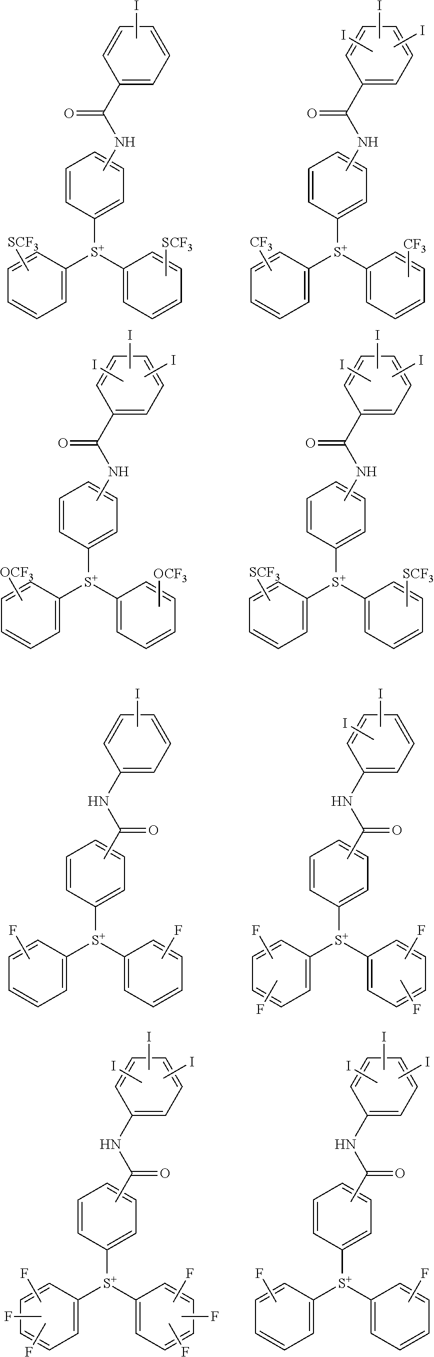

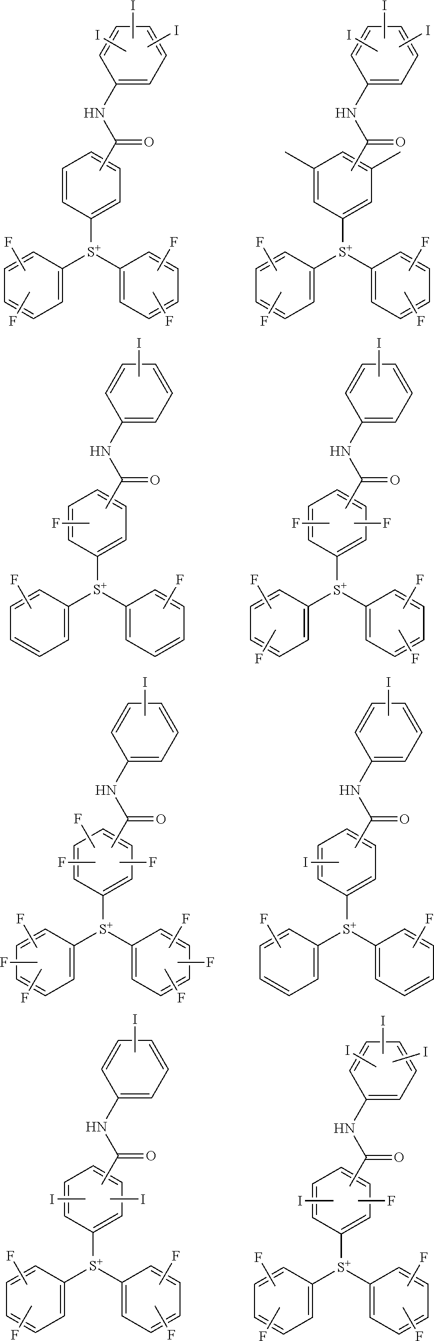

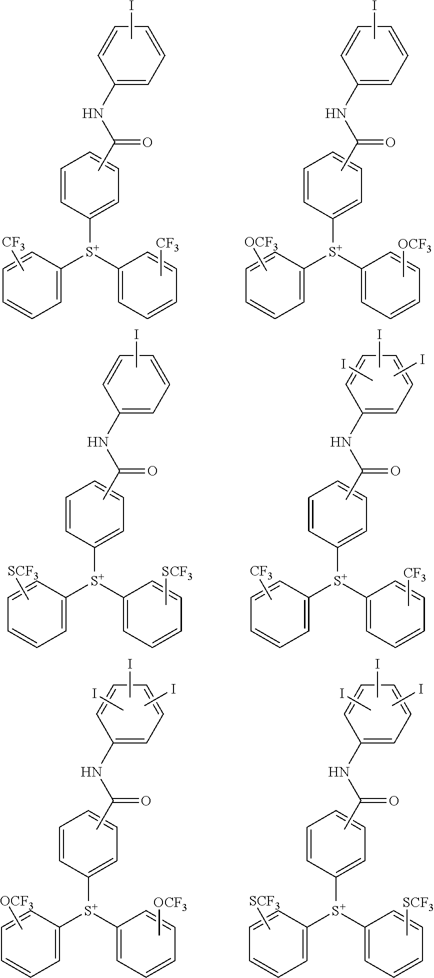

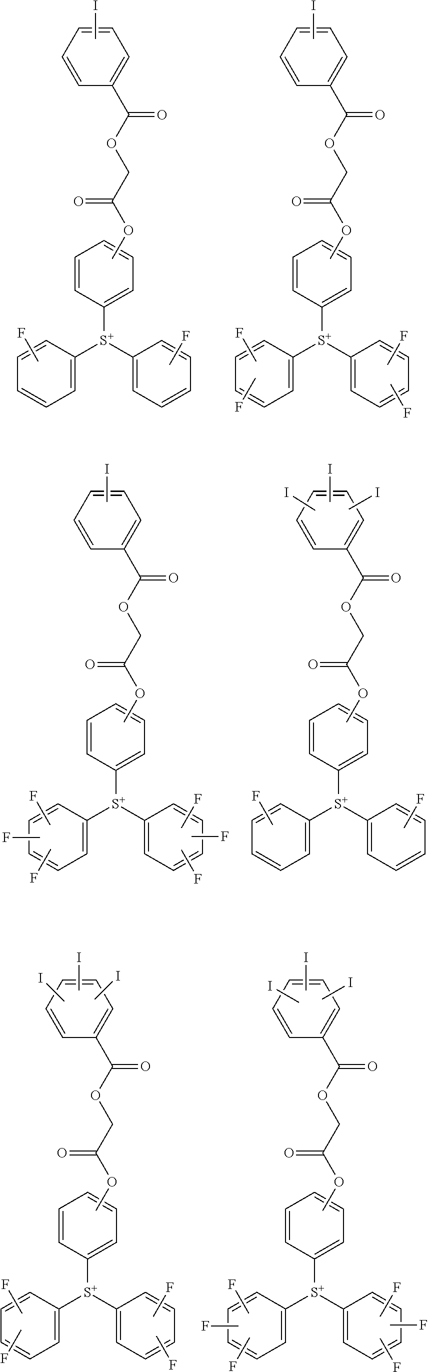

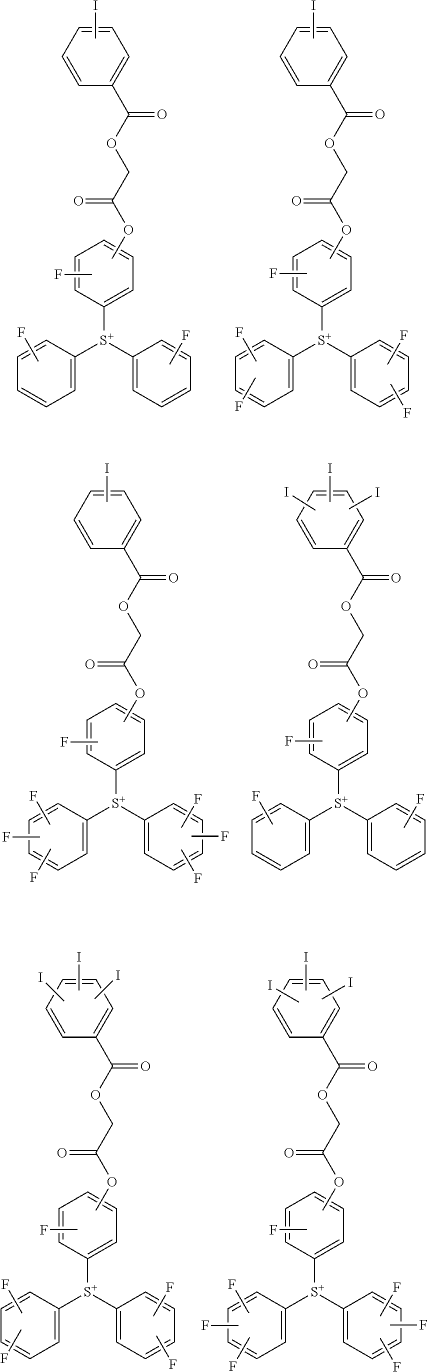

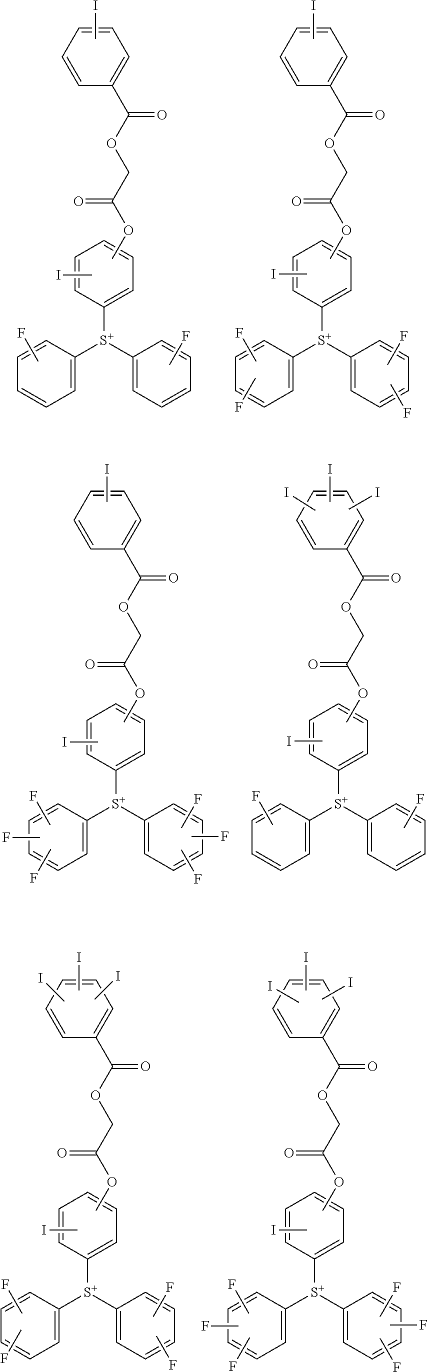

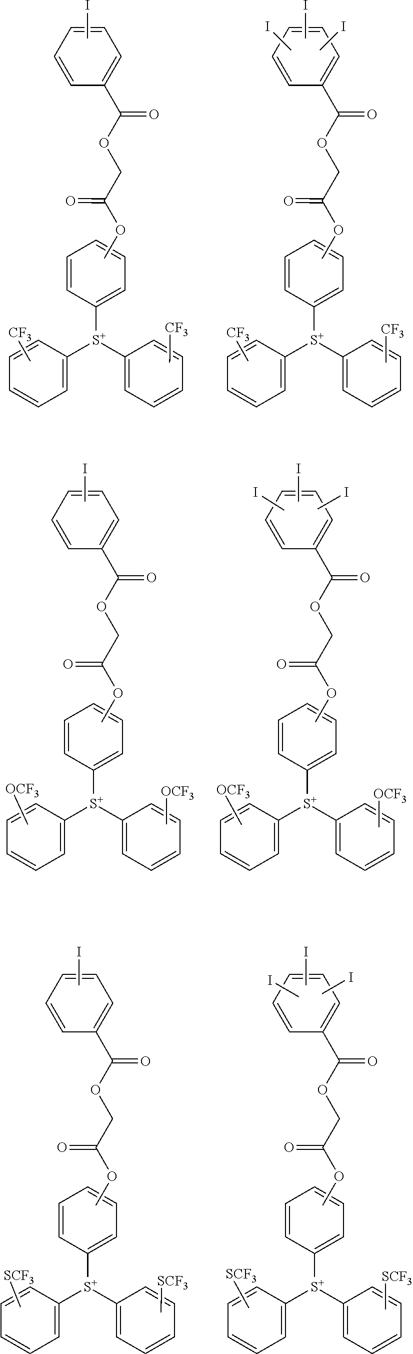

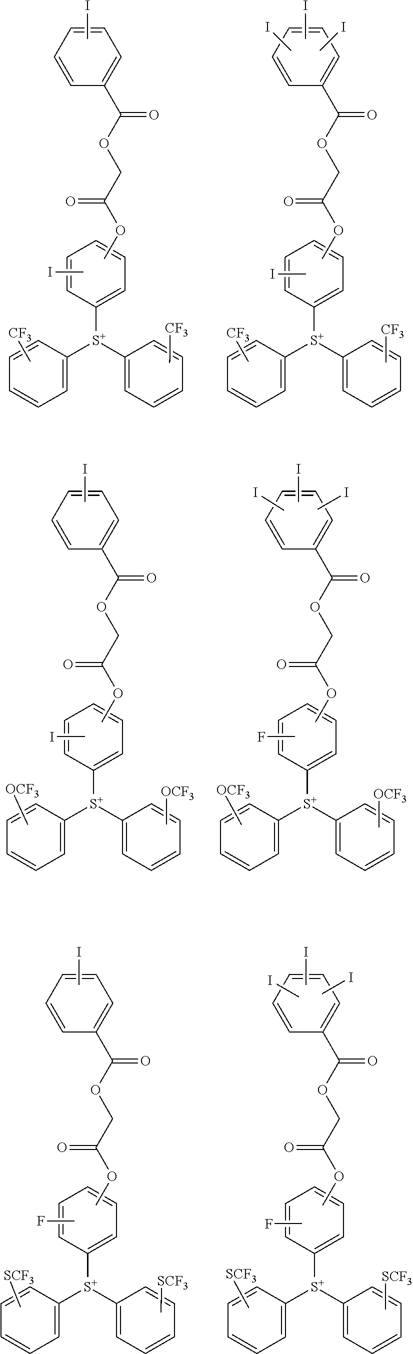

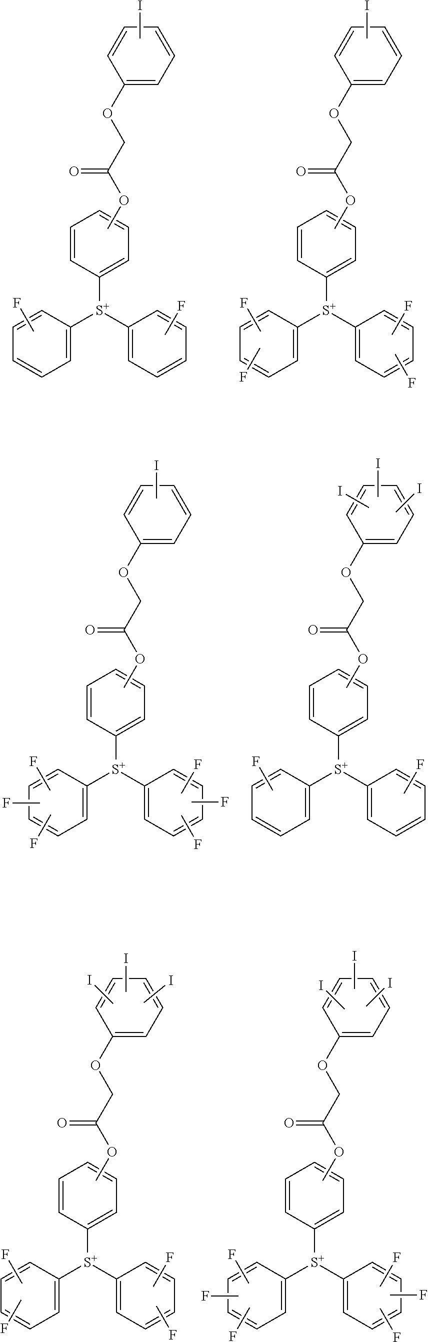

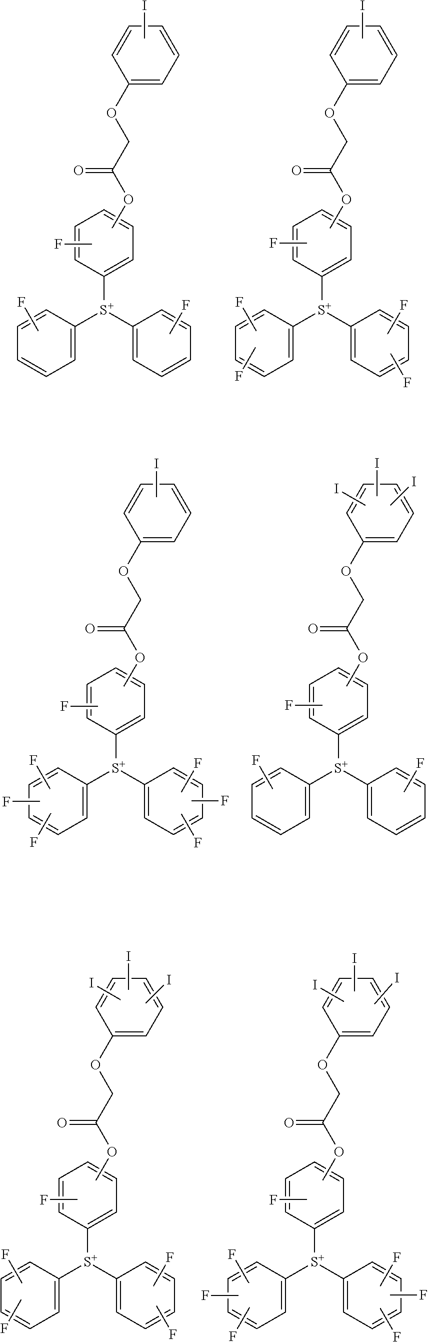

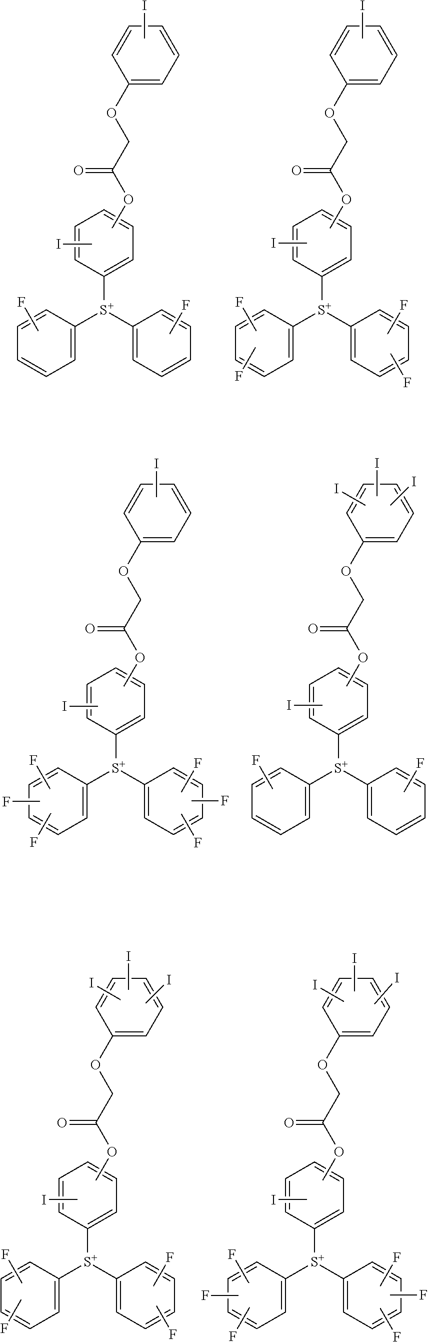

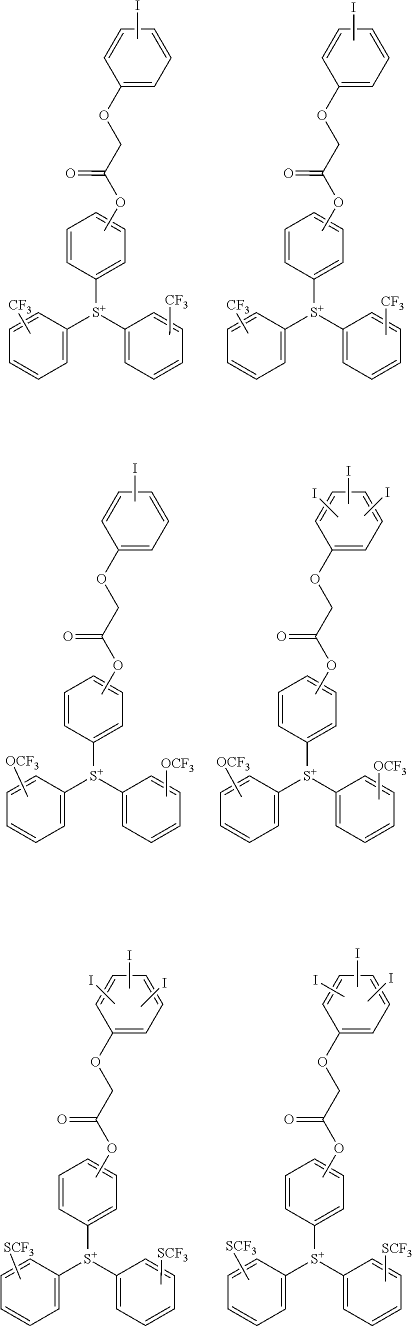

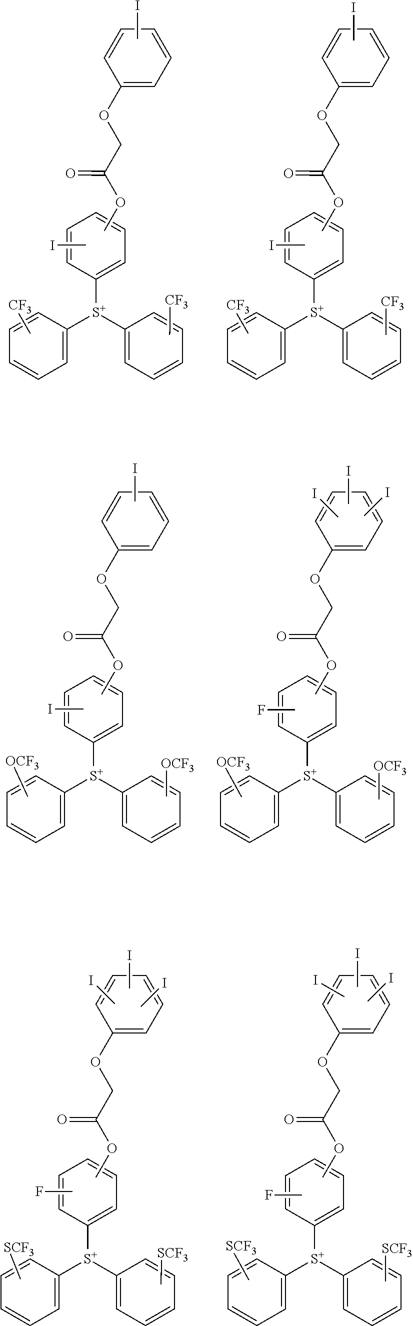

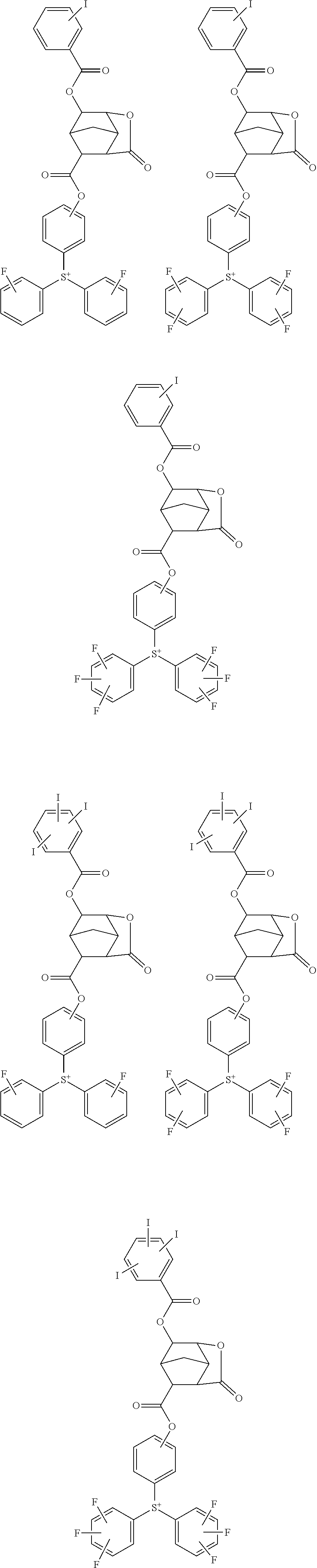

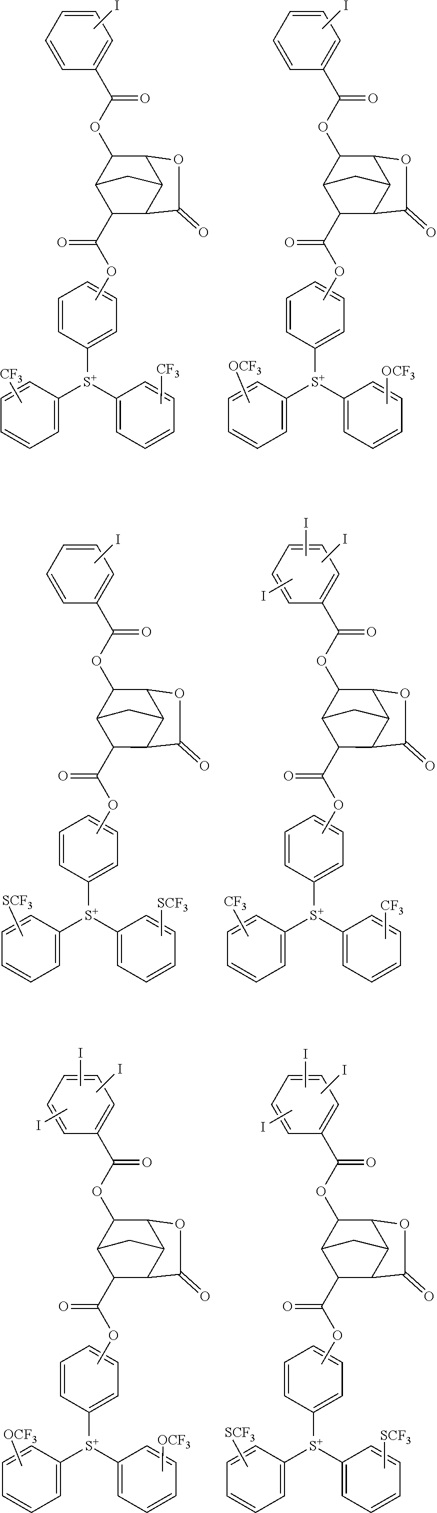

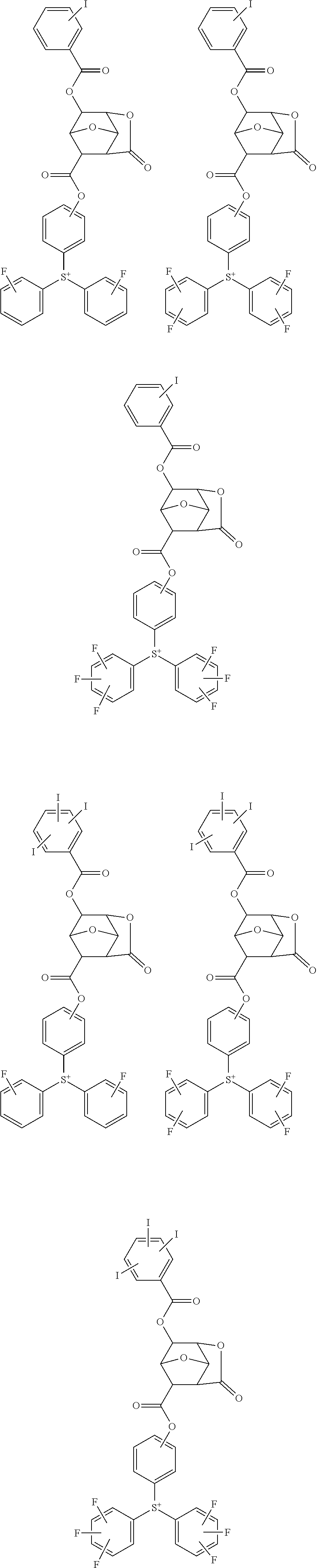

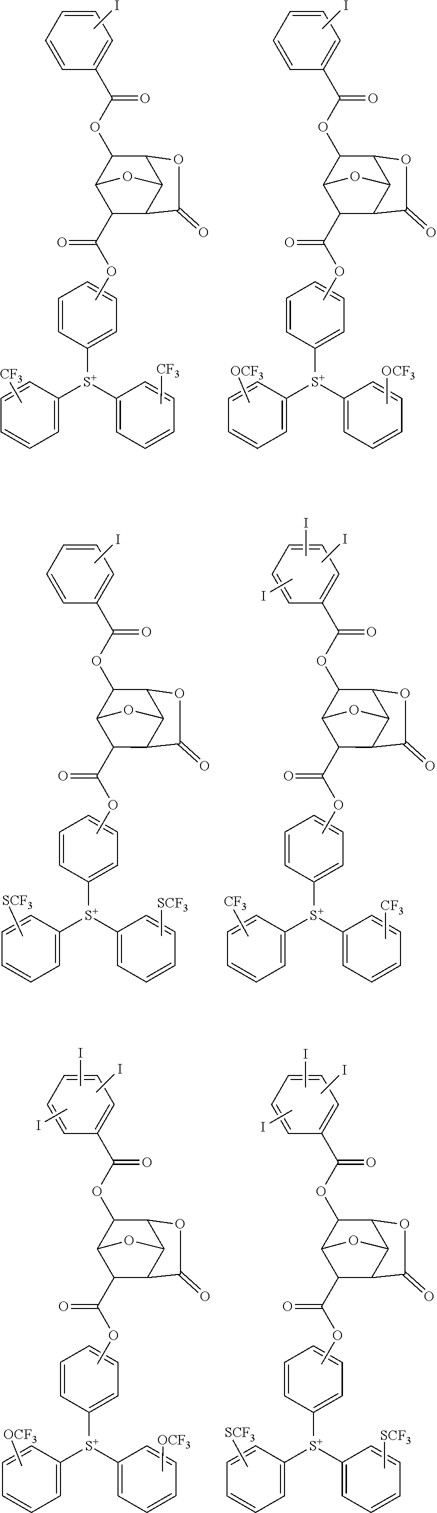

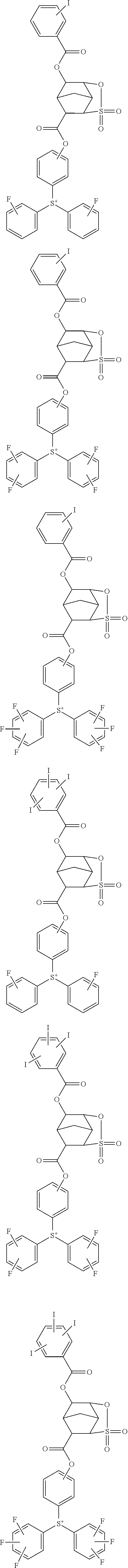

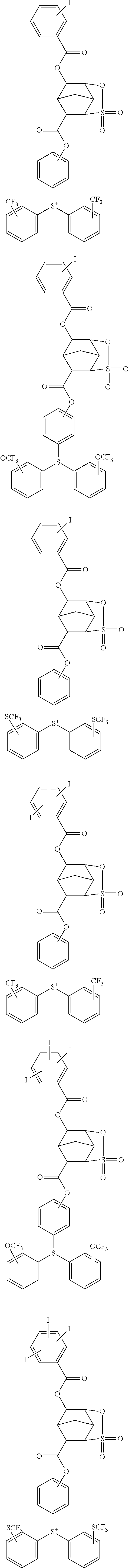

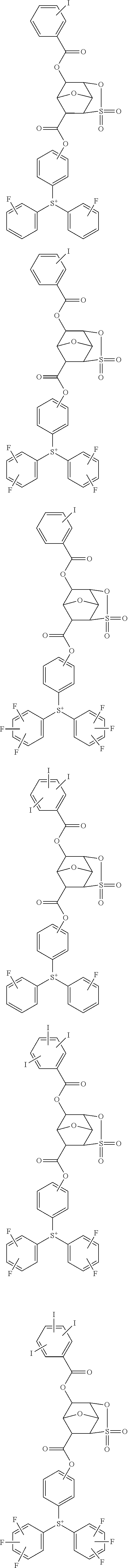

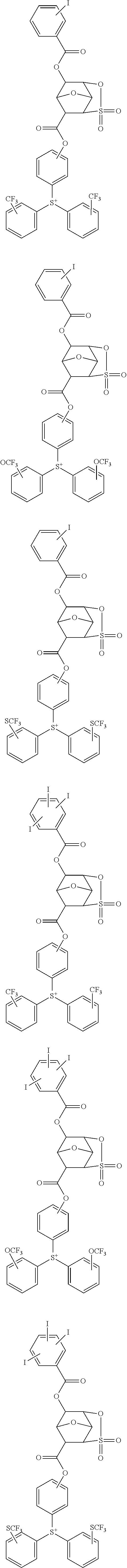

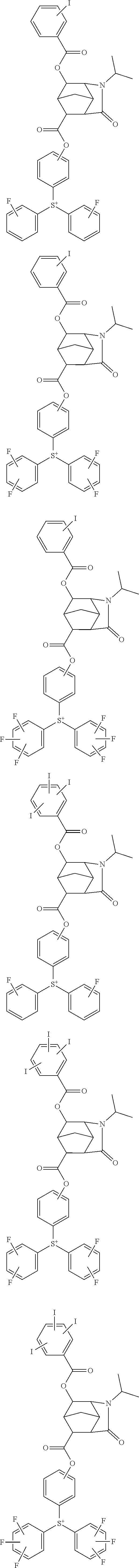

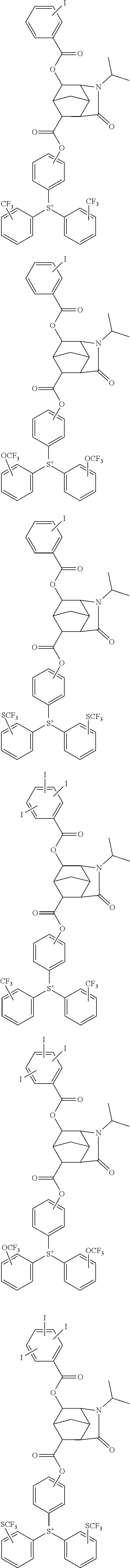

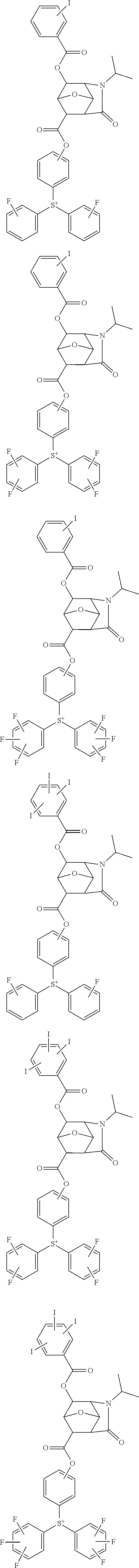

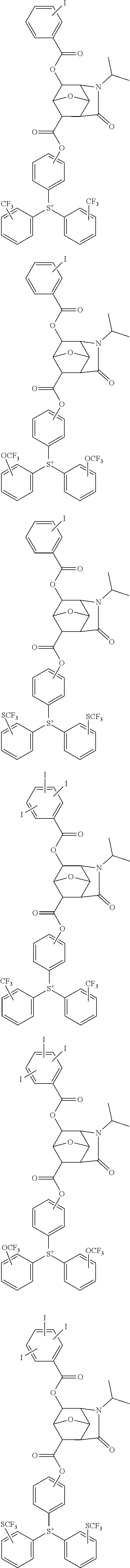

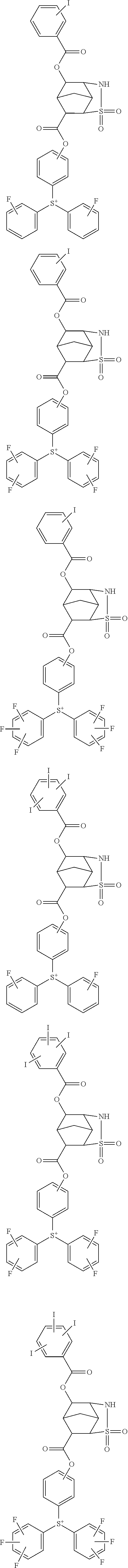

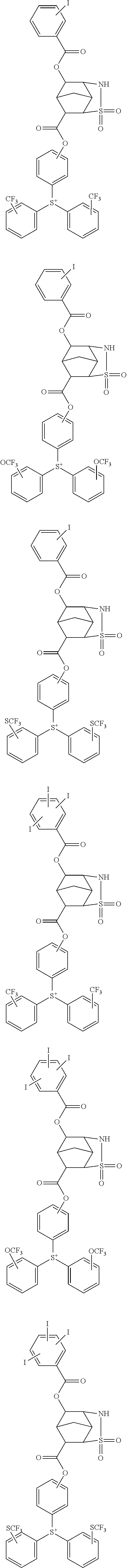

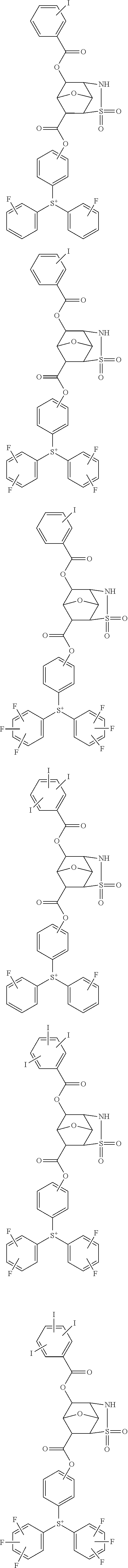

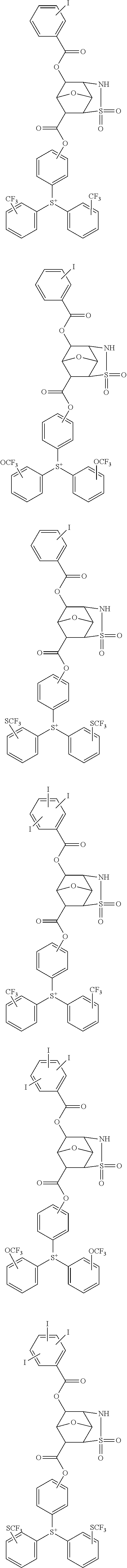

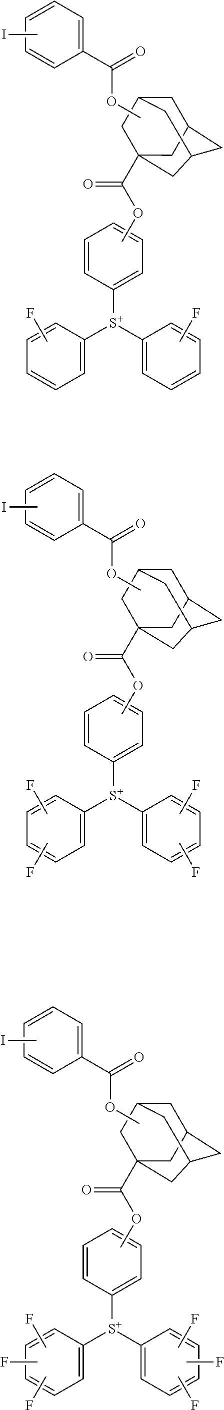

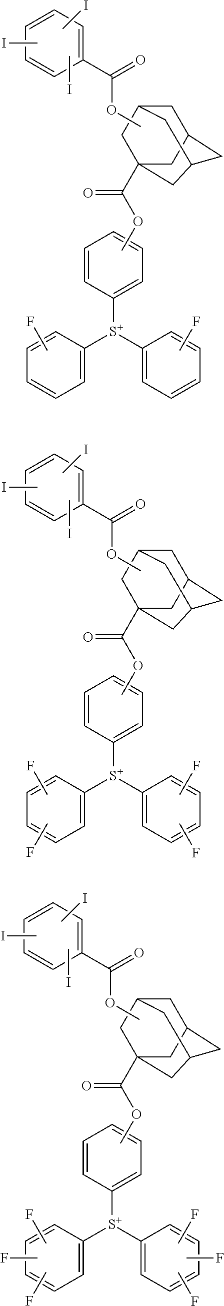

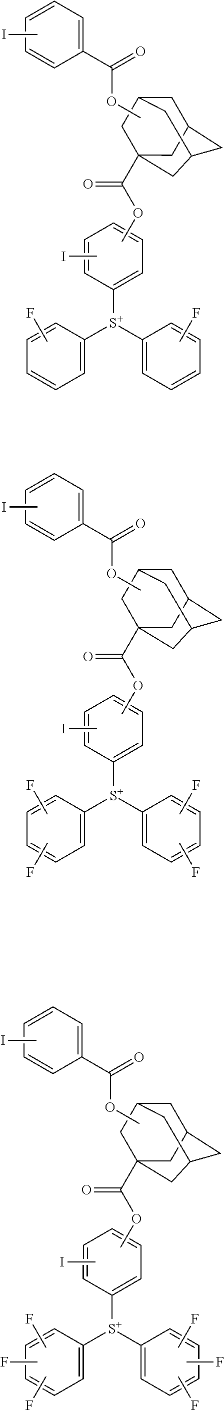

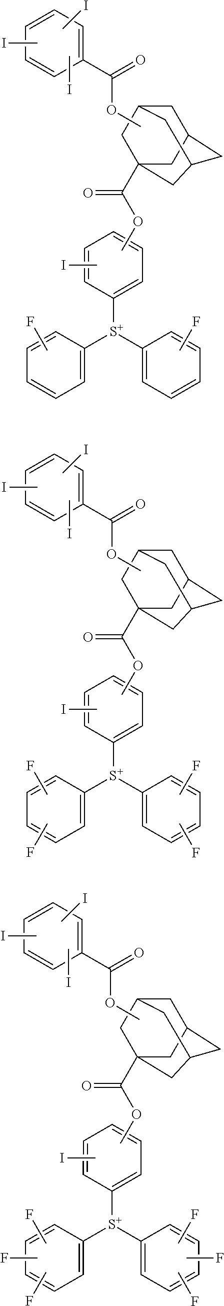

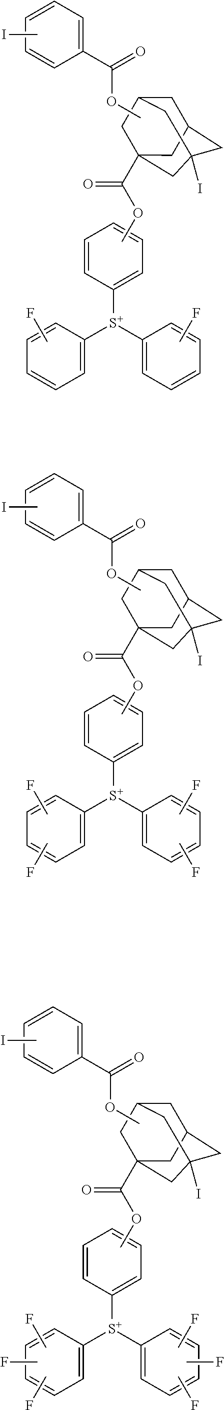

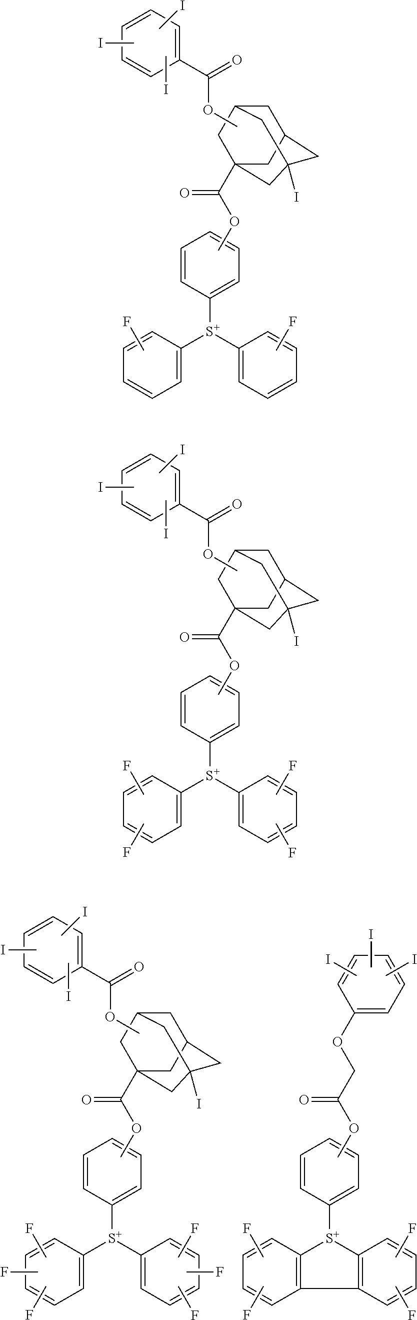

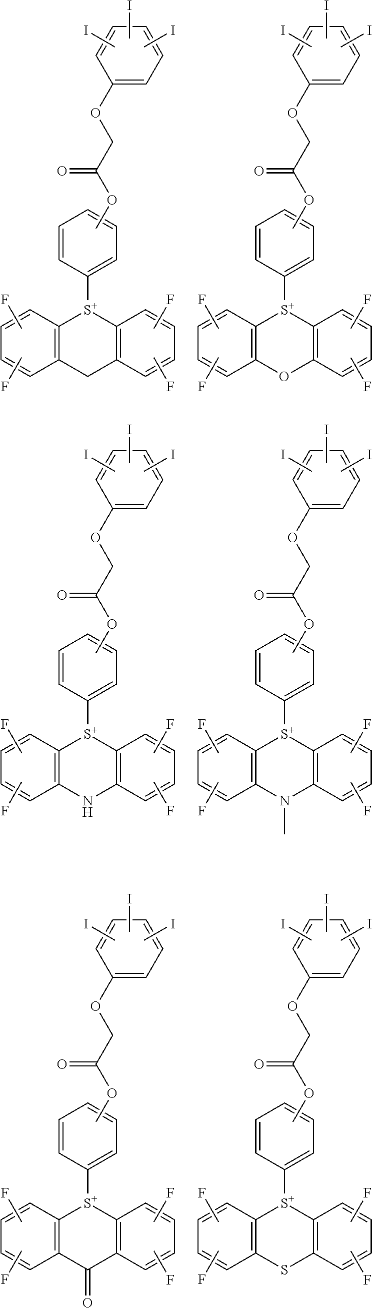

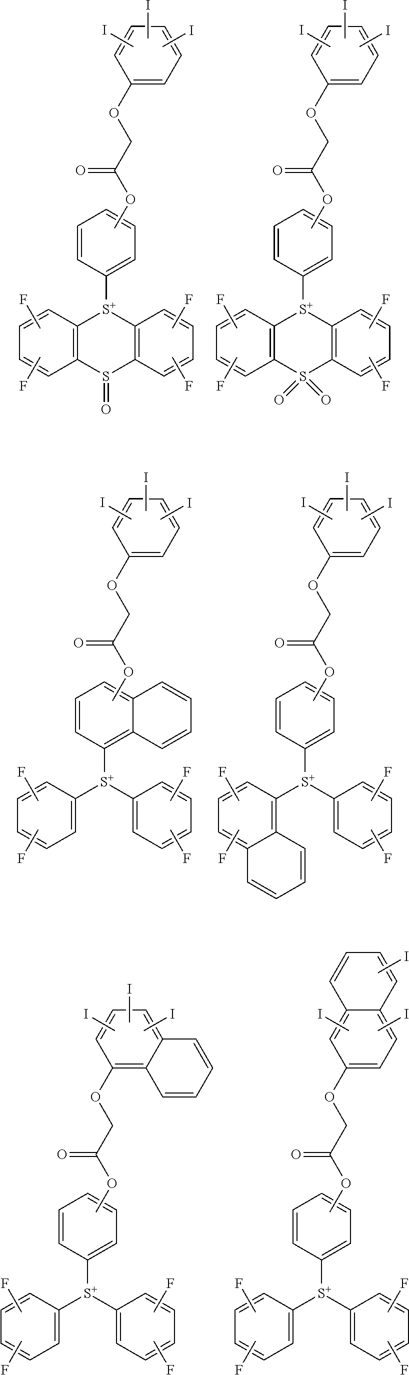

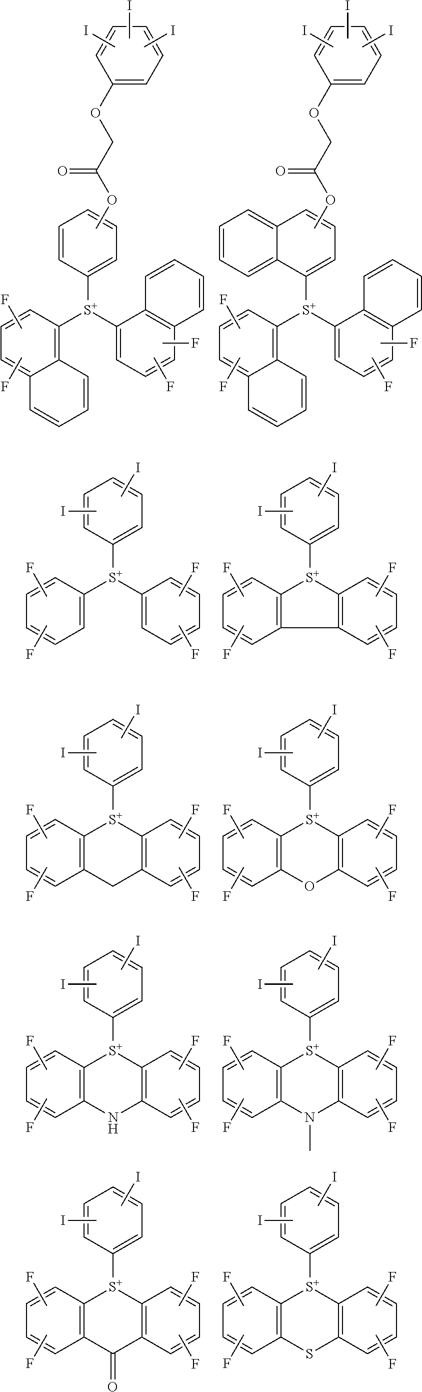

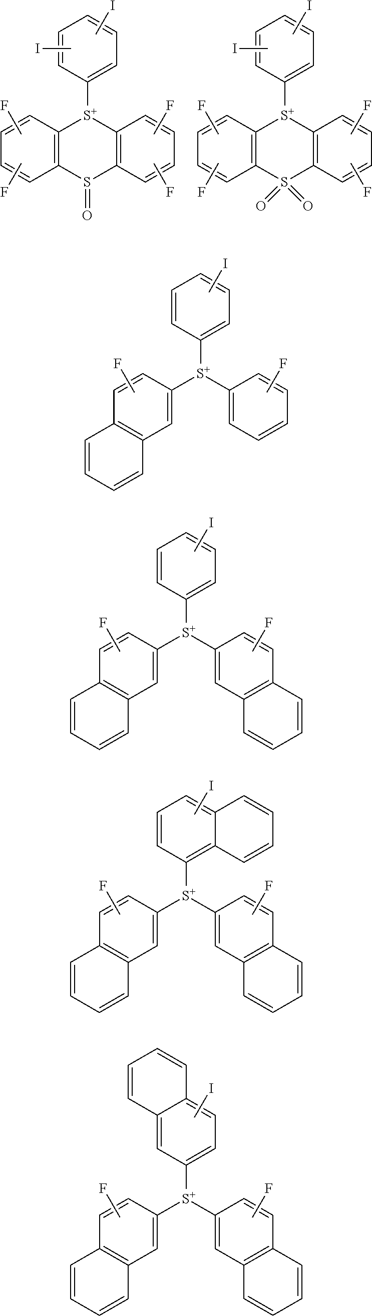

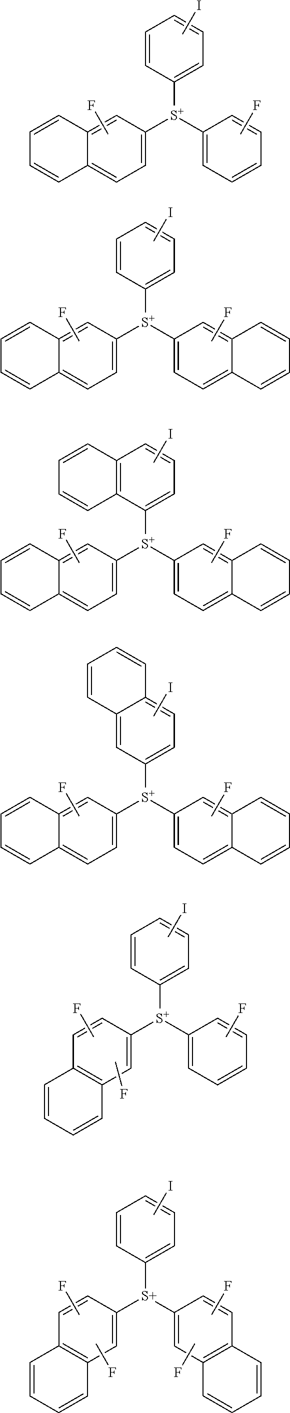

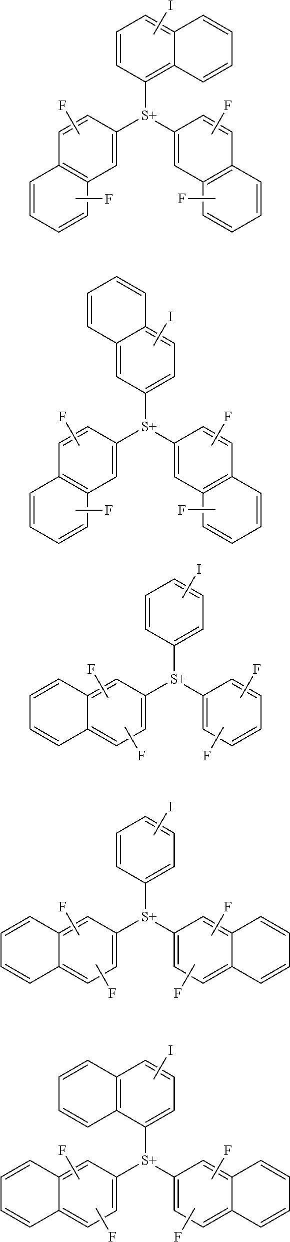

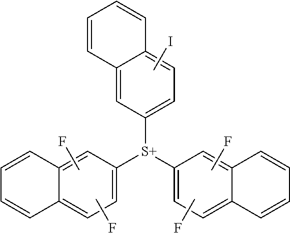

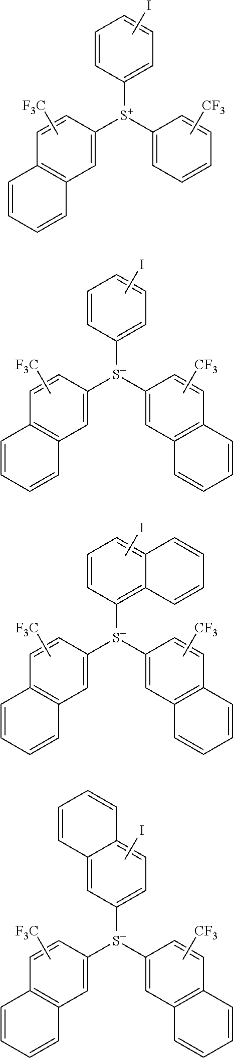

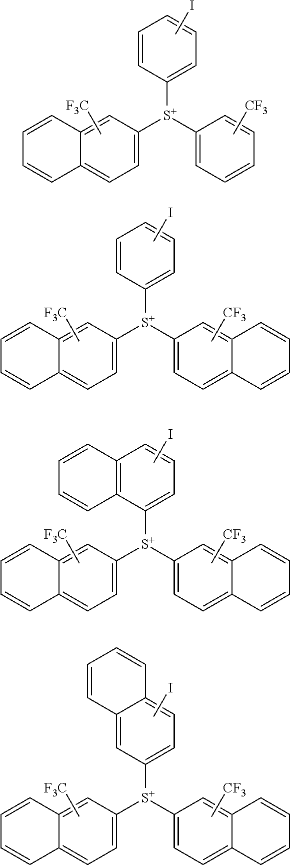

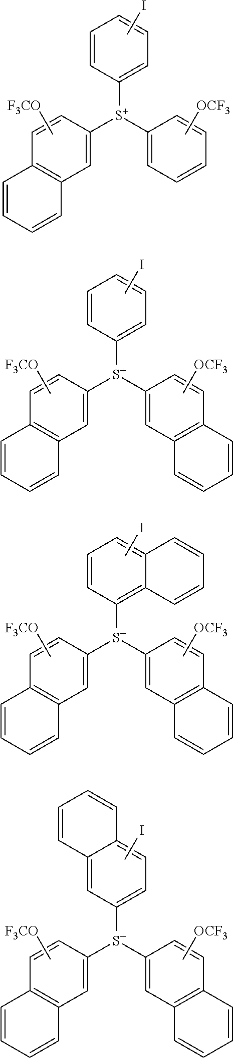

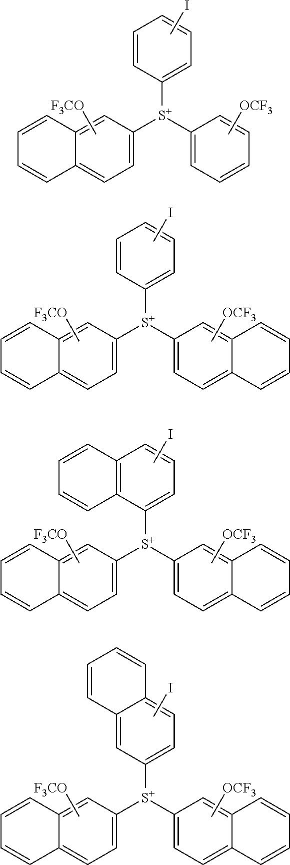

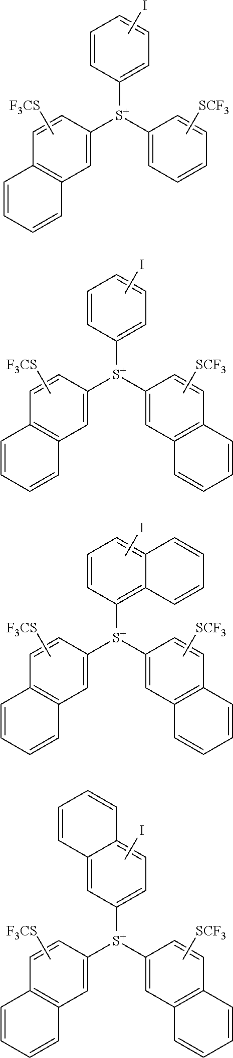

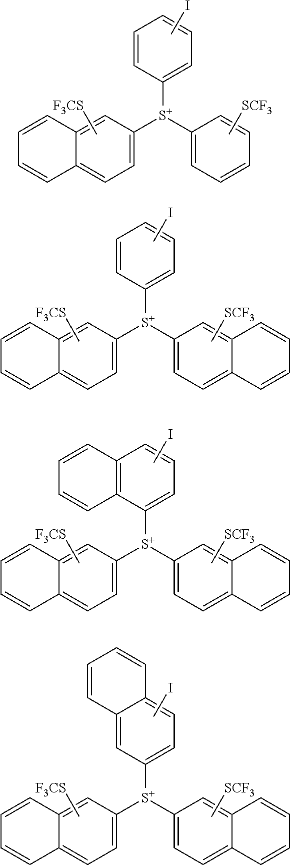

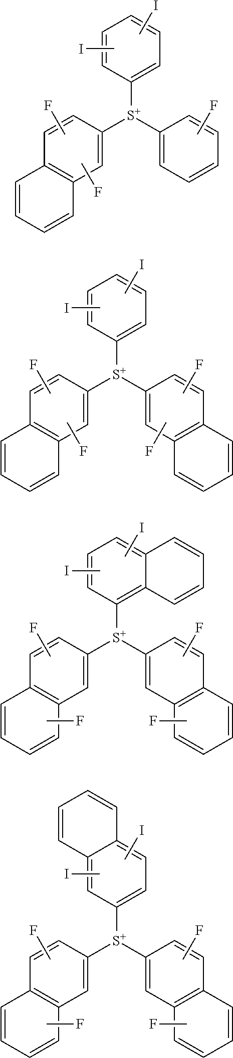

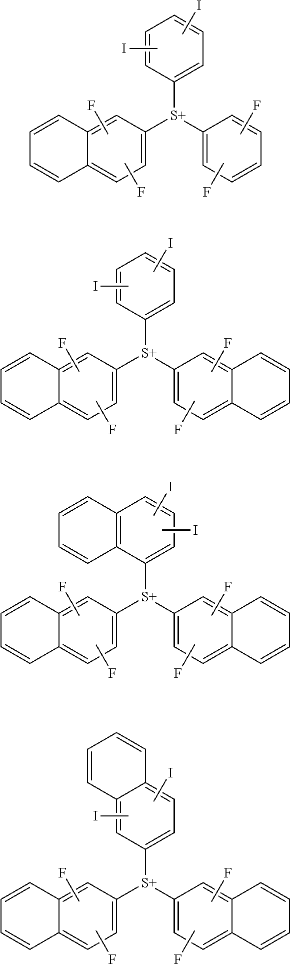

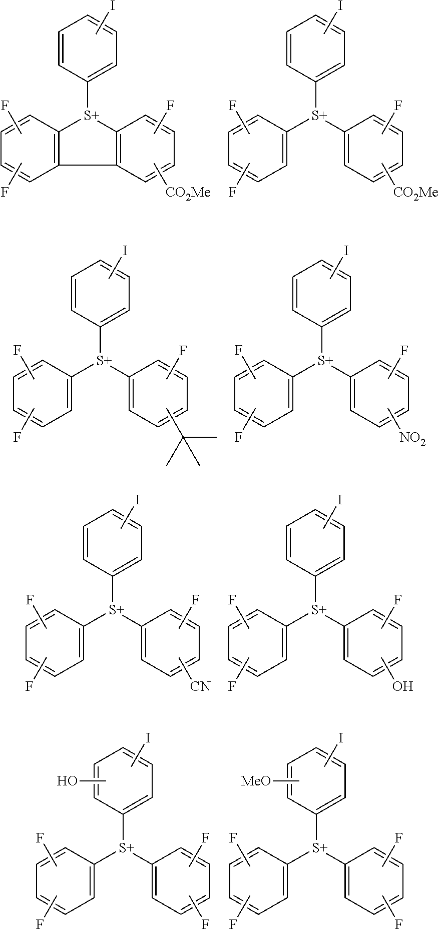

Examples of the onium salt monomer include arbitrary combinations of anions with cations, both as exemplified above.

The onium salt monomer can be synthesized, for example, by the method of preparing a sulfonium salt having a polymerizable anion, described in JP 5201363. This preparation method is merely exemplary and the method of preparing the inventive onium salt monomer is not limited thereto.

The onium salt monomer of the invention is useful as a starting material for a polymer-bound photoacid generator.

### [Polymer]

Another embodiment of the invention is a polymer comprising repeat units derived from the onium salt monomer having formula (A), which are referred to as units (A).

The polymer functions as a photoacid generator and a base polymer in a chemically amplified resist composition, that is, polymer-bound photoacid generator. The polymer is structurally characterized by having an acenaphthylene structure in the backbone and an aromatic sulfonic acid anion structure as side chain. Since the aromatic sulfonic acid anion structure is more robust than the fluoroalkane sulfonate structure, the acid diffusion distance can be reduced. On the other hand, acenaphthylene is a polycyclic aromatic hydrocarbon and has both robustness and polymerizability. Since an aromatic ring is included in the backbone, the polymer has a robust backbone and hence, a higher glass transition temperature (Tg). By introducing an acenaphthylene structure in the polymer backbone, the excessive diffusion of the acid generated after exposure is suppressed. Owing to the interaction of aromatic rings within or between the polymer (x-x stacking effect), the polymer is arranged in order. This ensures that when a resist film is developed in a developer to form a small-size pattern, the resist pattern is resistant to collapse in the developer. Also, in the etching step after the formation of a small-size pattern, the acenaphthylene structure contributes to better etching resistance. By virtue of the synergy of these effects, the excessive diffusion of the generated acid is suppressed. The inventive polymer can form patterns having improved lithography properties such as LWR of line patterns and CDU of hole patterns, and collapse resistance. The inventive polymer is thus useful as one component of a chemically amplified resist composition.

The polymer may further comprise repeat units having the formula (a1) or repeat units having the formula (a2). These units are also referred to as repeat units (a1) and (a2).

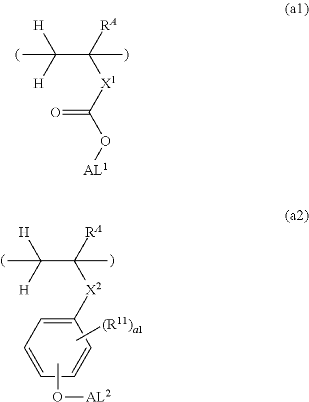

In formulae (a1) and (a2), R^{A} is each independently hydrogen, fluorine, methyl or trifluoromethyl.

In formula (a1), X¹ is a single bond, phenylene group, naphthylene group, *—C(═O)—O—X¹¹— or *—C(═O)—NH—X¹¹— wherein * designates a point of attachment to the carbon atom in the backbone. The phenylene and naphthylene groups may be substituted with hydroxy, nitro, cyano, an optionally fluorinated C₁-C₁₀ saturated hydrocarbyl moiety, optionally fluorinated C₁-C₁₀ saturated hydrocarbyloxy moiety, or halogen. X¹¹ is a C₁-C₁₀ saturated hydrocarbylene group, phenylene group or naphthylene group, and the saturated hydrocarbylene group may contain hydroxy, ether bond, ester bond or lactone ring.

In formula (a2), X² is a single bond, *—C(═O)—O— or *—C(═O)—NH—, wherein * designates a point of attachment to the carbon atom in the backbone. R¹¹ is halogen, cyano, hydroxy, nitro, a C₁-C₂₀ hydrocarbyl group which may contain a heteroatom, C₁-C₂₀ hydrocarbyloxy group which may contain a heteroatom, C₂-C₂₀ hydrocarbylcarbonyl group which may contain a heteroatom, C₂-C₂₀ hydrocarbylcarbonyloxy group which may contain a heteroatom, or C₂-C₂₀ hydrocarbyloxy carbonyl group which may contain a heteroatom, and a1 is 0, 1, 2, 3 or 4, preferably 0 or 1. When a1 is 2, 3 or 4, a plurality of R¹¹ may be identical or different.

In formulae (a1) and (a2), AL¹ and AL² are each independently an acid labile group. The acid labile group may be selected from a variety of such groups, for example, those groups described in JP-A 2013-080033 (U.S. Pat. No. 8,574,817) and JP-A 2013-083821 (U.S. Pat. No. 8,846,303).

Typical of the acid labile group are groups of the following formulae (AL-1) to (AL-3).

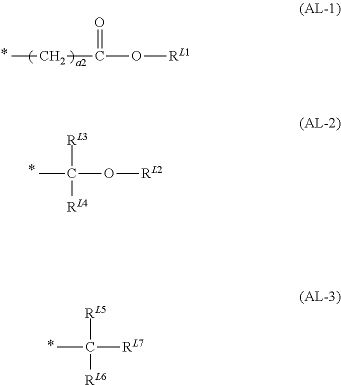

In formulae (AL-1) and (AL-2), R^{L1} and R^{L2} are each independently a C₁-C₄₀ hydrocarbyl group which may contain a heteroatom such as oxygen, sulfur, nitrogen or fluorine. The hydrocarbyl group may be saturated or unsaturated and straight, branched or cyclic. Inter alia, C₁-C₂₀ hydrocarbyl groups are preferred.

In formula (AL-1), a2 is an integer of 0 to 10, preferably 1, 2, 3, 4 or 5.

In formula (AL-2), R^{L3} and R^{L4} are each independently hydrogen or a C₁-C₂₀ hydrocarbyl group which may contain a heteroatom such as oxygen, sulfur, nitrogen or fluorine. The hydrocarbyl group may be saturated or unsaturated and straight, branched or cyclic. Any two of R^{L2}, R^{L3} and R^{L4} may bond together to form a C₃-C₂₀ ring with the carbon atom or carbon and oxygen atoms to which they are attached. The ring preferably contains 4 to 16 carbon atoms and is typically alicyclic.

In formula (AL-3), R^{L5}, R^{L6} and R^{L7} are each independently a C₁-C₂₀ hydrocarbyl group which may contain a heteroatom such as oxygen, sulfur, nitrogen or fluorine. The hydrocarbyl group may be saturated or unsaturated and straight, branched or cyclic. Any two of R^{L5}, R^{L6} and R^{L7} may bond together to form a C₃-C₂₀ ring with the carbon atom to which they are attached. The ring preferably contains 4 to 16 carbon atoms and is typically alicyclic.

Other examples of the acid labile group include those described in JP-A 2023-123222, paragraphs [0064]-[0068] and JP 7492842, paragraphs [0013]-[0014]. After acid elimination reaction, conjugated olefins or acrylate derivatives are produced and utilized as a driving force to forward the reaction.

Examples of the repeat unit (a1) are shown below, but not limited thereto. Herein R^{A} and A^{L1} are as defined above.

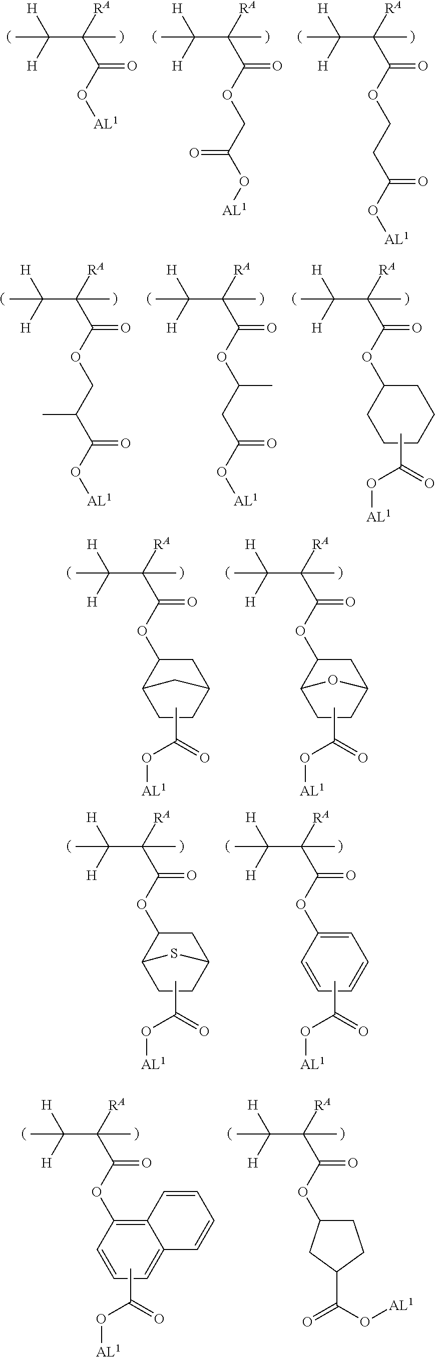

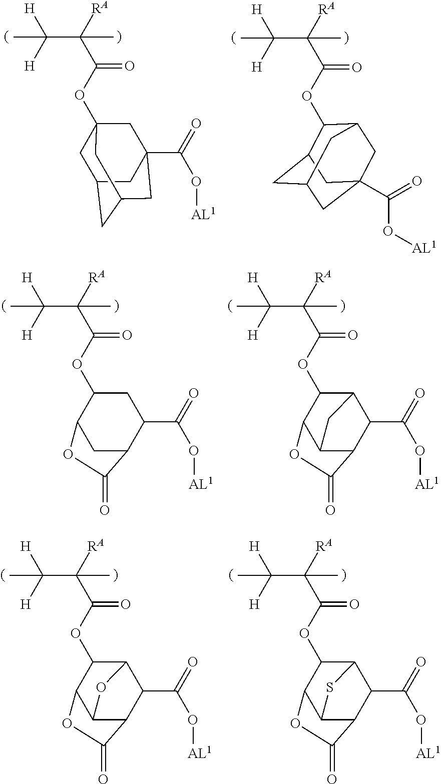

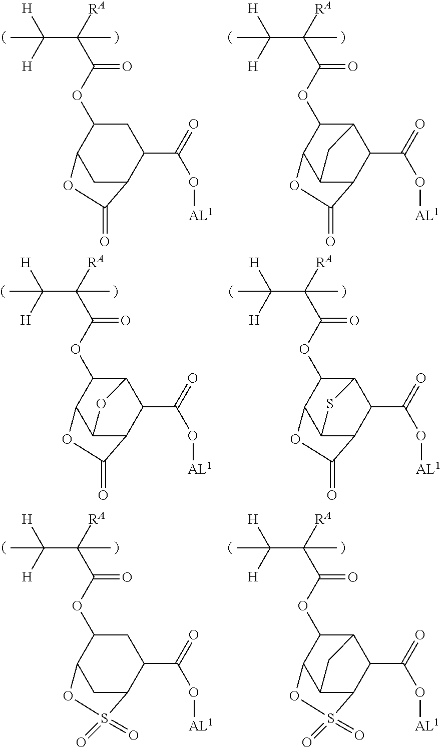

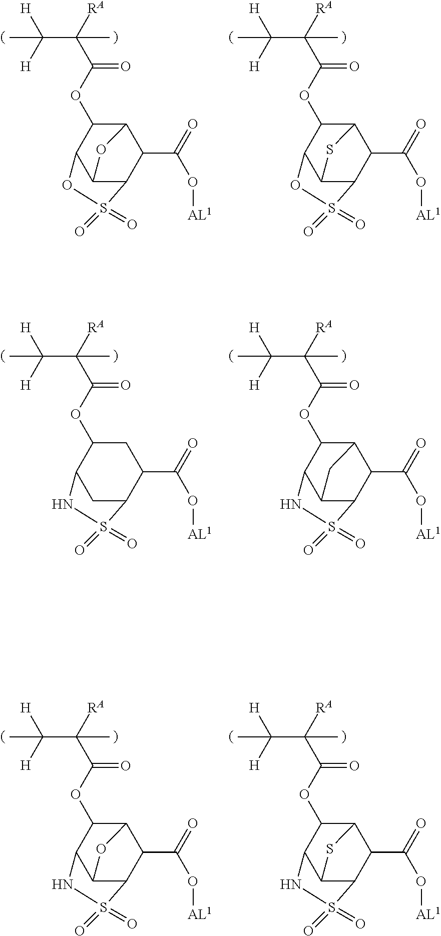

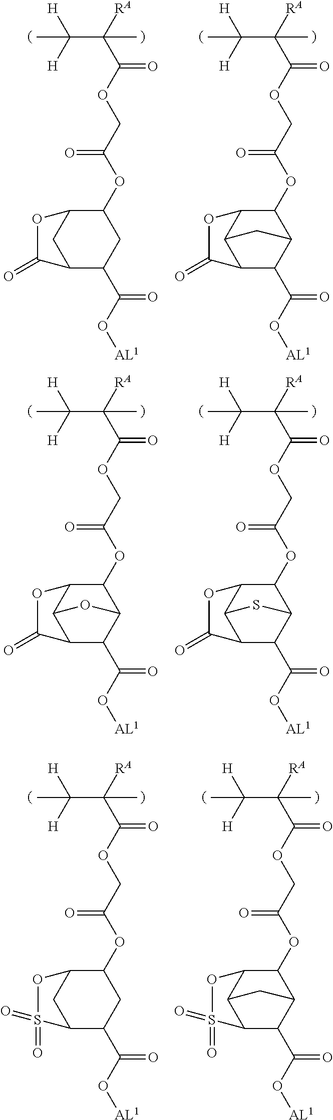

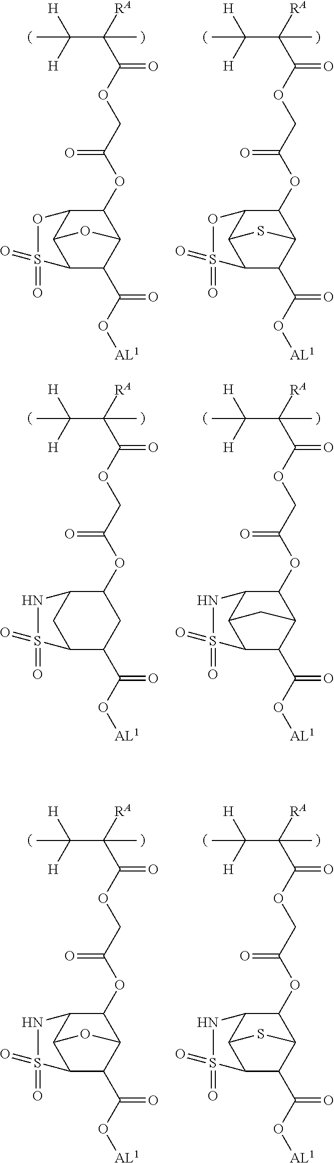

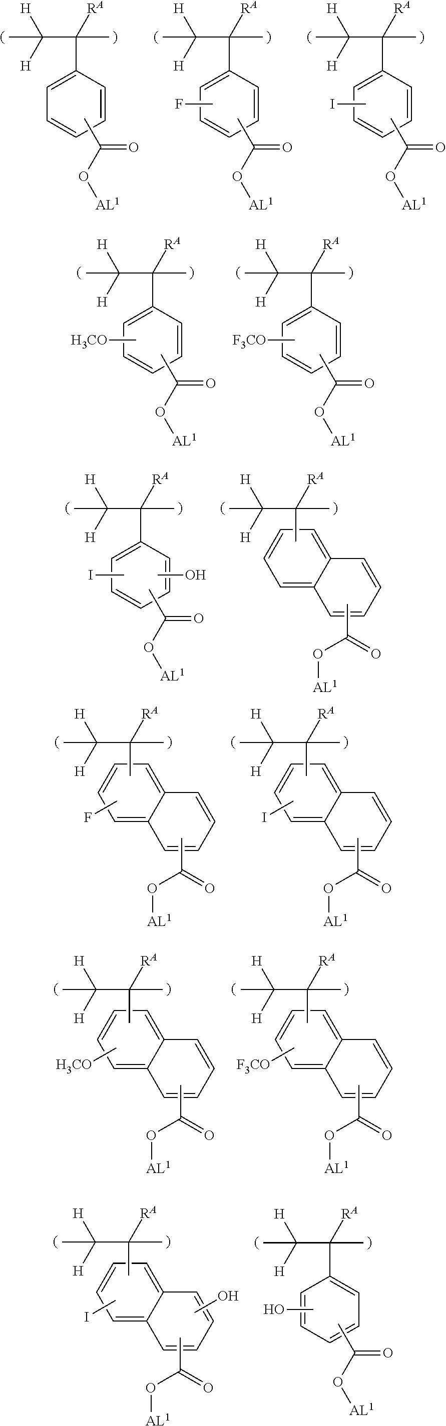

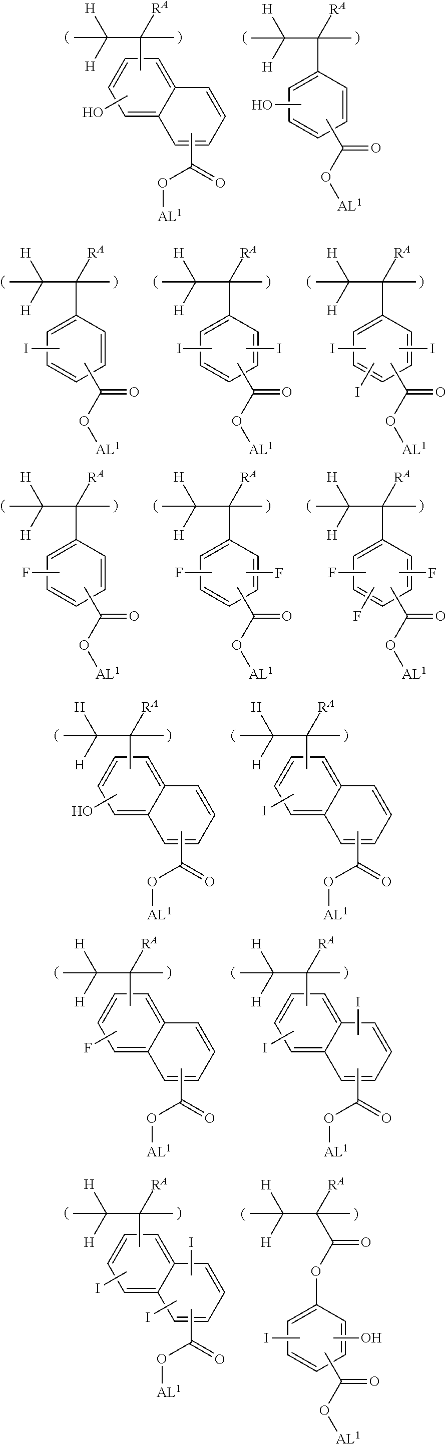

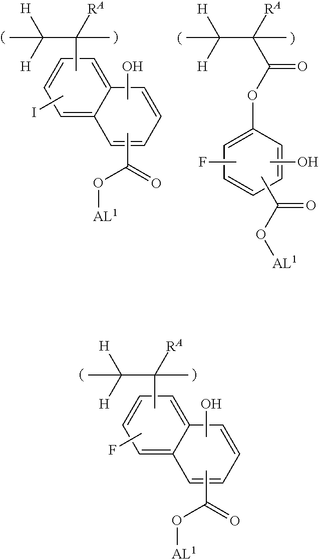

Examples of the repeat unit (a2) are shown below, but not limited thereto. Herein R^{A} and AL² are as defined above.

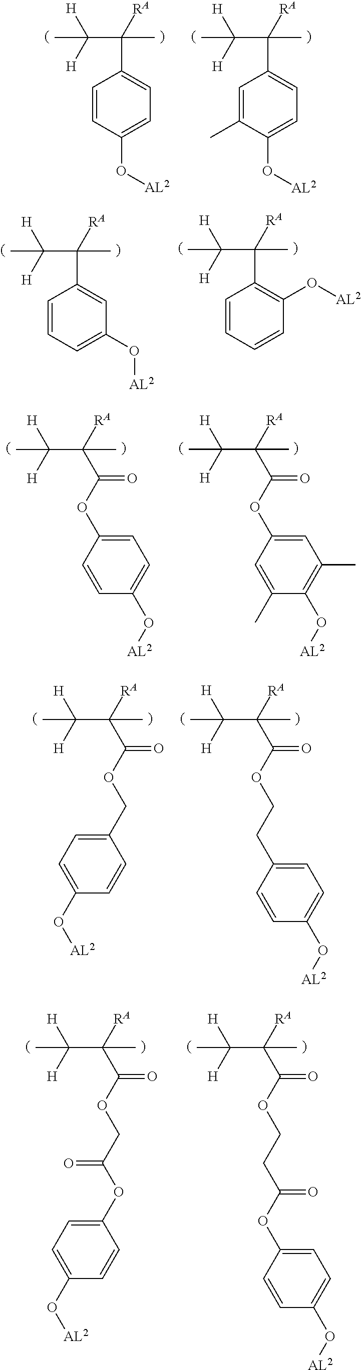

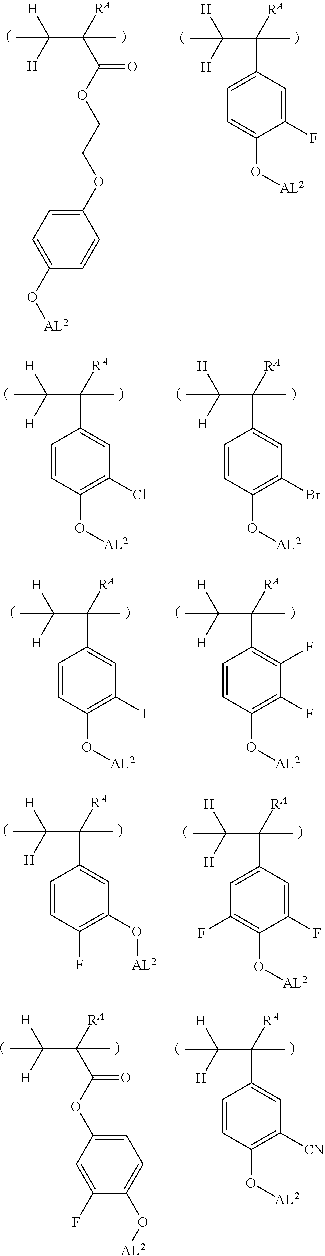

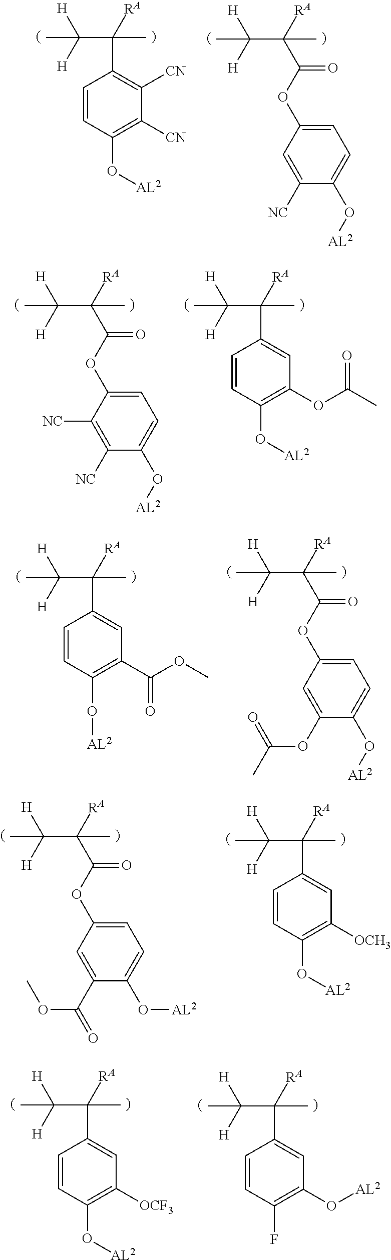

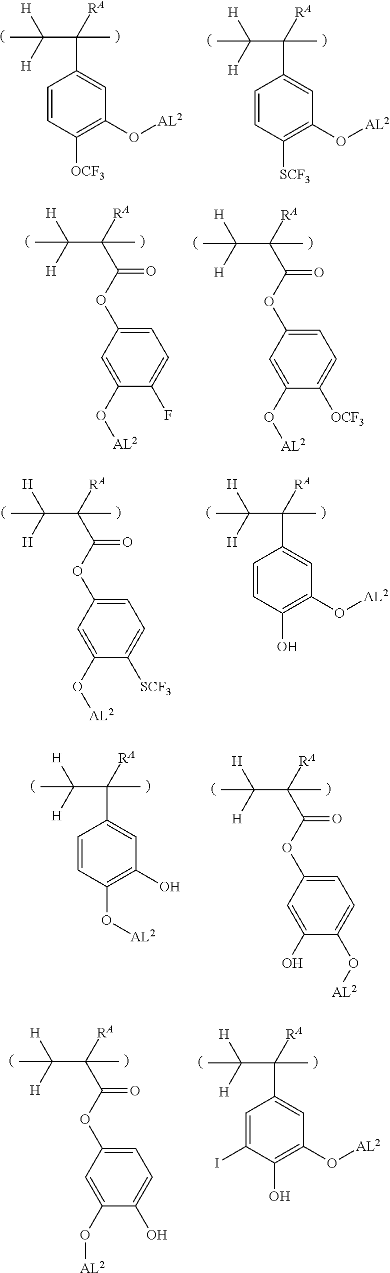

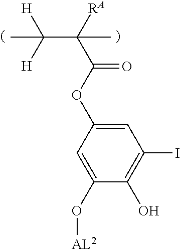

The polymer may further comprise repeat units having the formula (a3), which are also referred to as repeat units (a3).

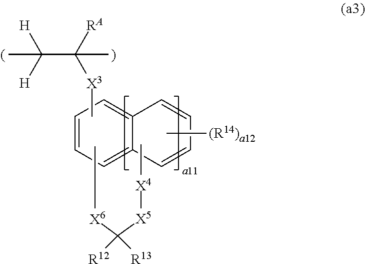

In formula (a3), all is 0 or 1. The relevant structure is a benzene ring in case of a11=0 and a naphthalene ring in case of a11=1. From the aspect of solvent solubility, the benzene ring corresponding to a11=0 is preferred. The subscript a12 is 0, 1, 2 or 3 when a11=0 and a12 is 0, 1, 2, 3, 4 or 5 when a11=1. It is preferred from the aspect of reactant availability that a12 be 0, 1, 2 or 3, more preferably 0, 1 or 2.

In formula (a3), R^{A} is hydrogen, fluorine, methyl or trifluoromethyl, preferably hydrogen or methyl, most preferably hydrogen.

In formula (a3), X³ is a single bond, *—C(═O)—O— or *—C(═O)—NH—, wherein * designates a point of attachment to the carbon atom in the backbone. X³ is preferably a single bond or *—C(═O)—O—, more preferably a single bond.

In formula (a3), X⁴ is a single bond, C₁-C₄ aliphatic hydrocarbylene group, carbonyl group, sulfonyl group or a group obtained by combining the foregoing. It is preferred from the aspect of reactant availability that X⁴ be a single bond, carbonyl or sulfonyl. It is more preferred from the aspect of a polar group created after reaction that X⁴ be a single bond or carbonyl.

In formula (a3), X⁵ and X⁶ are each independently oxygen or sulfur, with the proviso that X⁴ and X⁶ are attached to vicinal carbon atoms on the aromatic ring. X⁵ and X⁶ may be the same or different. It is preferred from the aspect of reactivity that X⁵ and X⁶ be both oxygen.

In formula (a3), R¹² and R¹³ are each independently hydrogen or a C₁-C₂₀ hydrocarbyl group which may contain a heteroatom. The hydrocarbyl group may be saturated or unsaturated and straight, branched or cyclic. Examples thereof include C₁-C₂₀ alkyl groups such as methyl, ethyl, n-propyl, isopropyl, n-butyl, isobutyl, sec-butyl, tert-butyl, n-pentyl, n-hexyl, n-octyl, n-nonyl, n-decyl, undecyl, dodecyl, tridecyl, tetradecyl, pentadecyl, heptadecyl, octadecyl, nonadecyl, and icosyl: C₃-C₂₀ cyclic saturated hydrocarbyl groups such as cyclopropyl, cyclopentyl, cyclohexyl, cyclopropylmethyl, 4-methylcyclohexyl, cyclohexylmethyl, norbomnyl, and adamantyl: C₂-C₂₀ alkenyl groups such as vinyl, 1-propenyl, 2-propenyl, butenyl, and hexenyl: C₃-C₂₀ cyclic unsaturated hydrocarbyl groups such as cyclohexenyl: C₆-C₂₀ aryl groups such as phenyl and naphthyl; C₇-C₂₀ aralkyl groups such as benzyl, 1-phenylethyl, and 2-phenylethyl, and combinations thereof. In the hydrocarbyl group, some or all of the hydrogen atoms may be substituted by a moiety containing a heteroatom such as oxygen, sulfur, nitrogen or halogen, and some constituent —CH₂— may be replaced by a moiety containing a heteroatom such as oxygen, sulfur or nitrogen, so that the group may contain a hydroxy moiety, cyano moiety, fluorine, chlorine, bromine, iodine, carbonyl moiety, ether bond, ester bond, sulfonate ester bond, carbonate bond, lactone ring, sultone ring, carboxylic anhydride (—C(═O)—O—C(═O)—) or haloalkyl moiety.

Also, R¹² and R¹³ may bond together to form a ring with the carbon atom to which they are attached. Examples of the ring include cyclopropane, cyclobutane, cyclopentane, cyclohexane, norbornane, and adamantane rings. In the ring, some or all of the hydrogen atoms may be substituted by a moiety containing a heteroatom such as oxygen, sulfur, nitrogen or halogen, and some constituent —CH₂— may be replaced by a moiety containing a heteroatom such as oxygen, sulfur or nitrogen, so that the ring may contain a hydroxy moiety, fluorine, chlorine, bromine, iodine, cyano moiety, carbonyl moiety, ether bond, ester bond, sulfonate ester bond, carbonate bond, lactone ring, sultone ring, carboxylic anhydride (—C(═O)—O—C(═O)—) or haloalkyl moiety.

In formula (a3), R¹⁴ is halogen, hydroxy, cyano, nitro, a C₁-C₂₀ hydrocarbyl group which may contain a heteroatom, C₁-C₂₀ hydrocarbyloxy group which may contain a heteroatom, C₂-C₂₀ hydrocarbyloxycarbonyl group which may contain a heteroatom, C₁-C₂₀ hydrocarbylthio group which may contain a heteroatom, or —N(R^{14A})(R^{14B}). R^{14A} and R^{14B} are each independently hydrogen or a C₁-C₆ hydrocarbyl group. Suitable halogen atoms include fluorine, chlorine, bromine and iodine, with fluorine and iodine being preferred. The hydrocarbyl group and hydrocarbyl moiety in the hydrocarbyloxy, hydrocarbyloxycarbonyl, and hydrocarbylthio groups may be saturated or unsaturated and straight, branched or cyclic. Examples thereof are as exemplified above for the hydrocarbyl groups R¹² and R¹³. In the hydrocarbyl group, some or all of the hydrogen atoms may be substituted by a moiety containing a heteroatom such as oxygen, sulfur, nitrogen or halogen, and some constituent —CH₂— may be replaced by a moiety containing a heteroatom such as oxygen, sulfur or nitrogen, so that the group may contain a hydroxy moiety, cyano moiety, fluorine, chlorine, bromine, iodine, carbonyl moiety, ether bond, ester bond, sulfonate ester bond, carbonate bond, lactone ring, sultone ring, carboxylic anhydride (—C(═O)—O—C(═O)—) or haloalkyl moiety. A plurality of R¹⁴ may be identical or different when a12 is 2 or more.

When a12 is 2 or more, a plurality of R¹⁴ may bond together to form a ring with carbon atoms in the aromatic ring to which they are attached. Examples of the ring include cyclopropane, cyclobutane, cyclopentane, cyclohexane, norbornane, and adamantane rings. In the ring, some or all of the hydrogen atoms may be substituted by a moiety containing a heteroatom such as oxygen, sulfur, nitrogen or halogen, and some constituent —CH₂— may be replaced by a moiety containing a heteroatom such as oxygen, sulfur or nitrogen, so that the ring may contain a hydroxy moiety, fluorine, chlorine, bromine, iodine, cyano moiety, carbonyl moiety, ether bond, ester bond, sulfonic ester bond, carbonate bond, lactone ring, sultone ring, carboxylic anhydride (—C(═O)—O—C(═O)—) or haloalkyl moiety.

Examples of repeat unit (a3) are shown below, but not limited thereto. Herein R^{A} is as defined above. The positions of attachment of substituent groups on the aromatic ring are interchangeable.

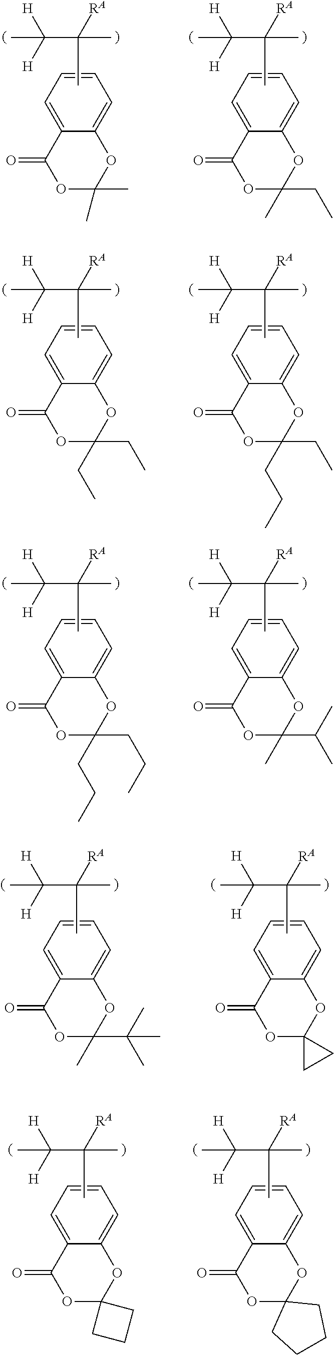

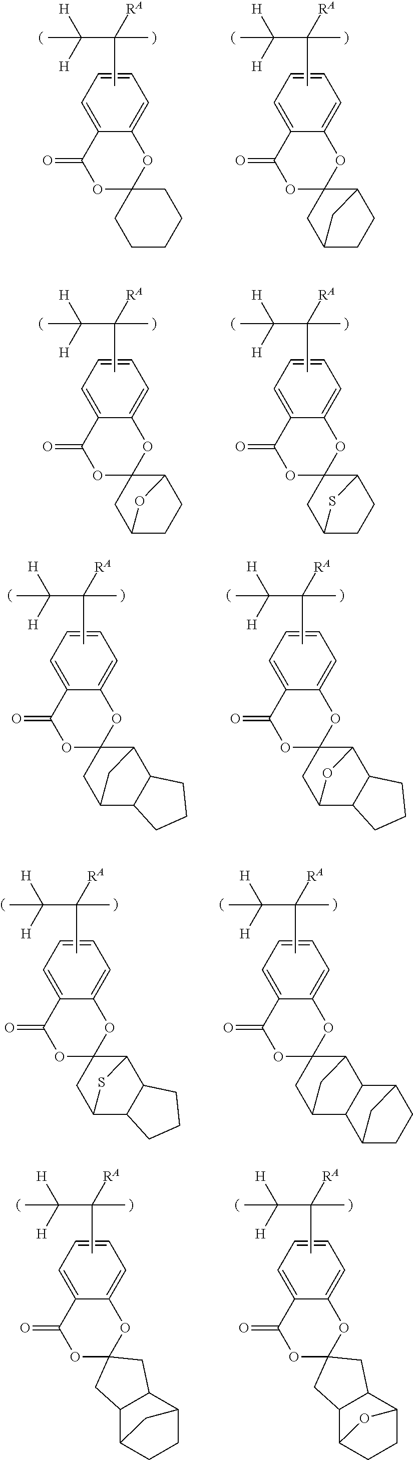

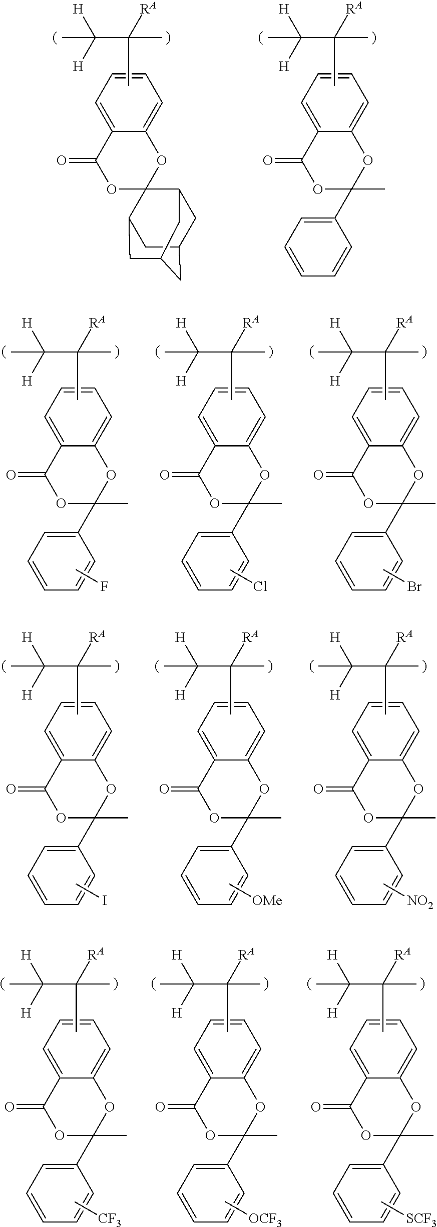

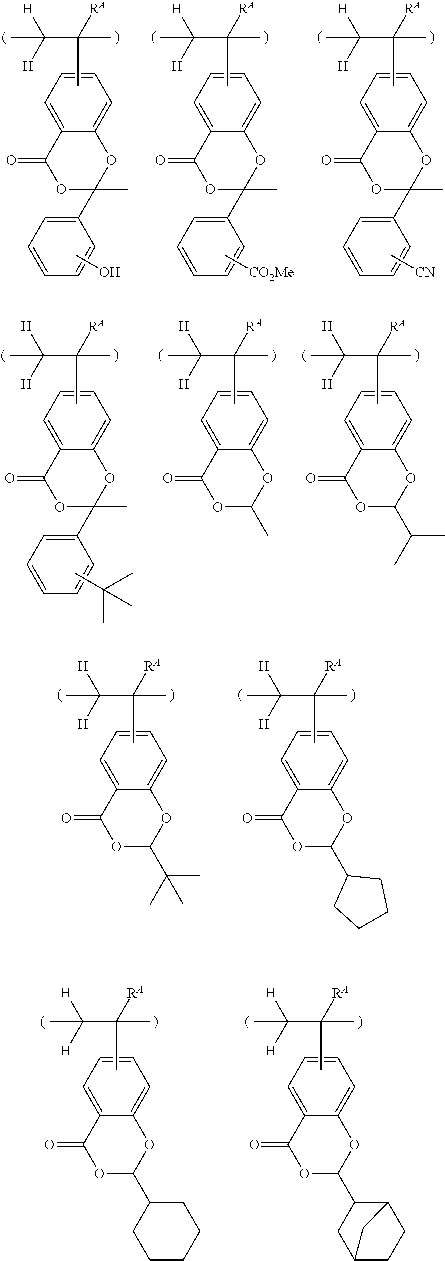

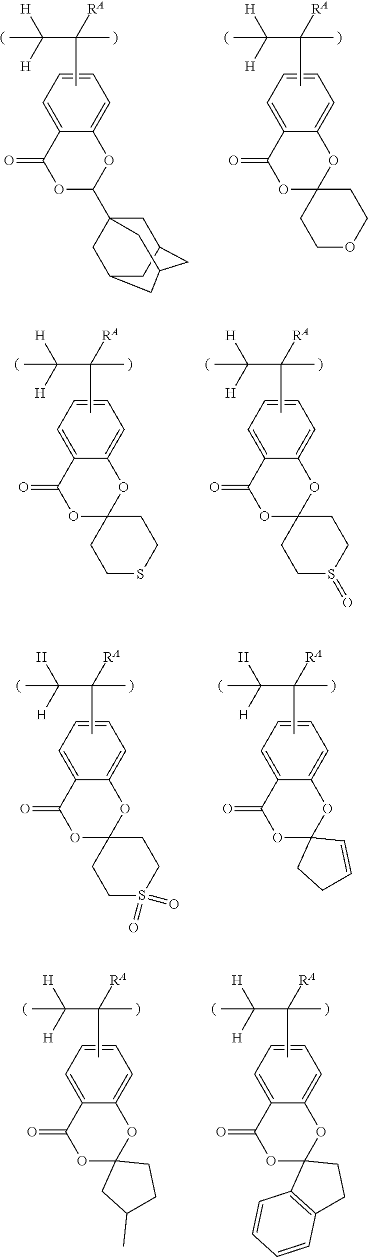

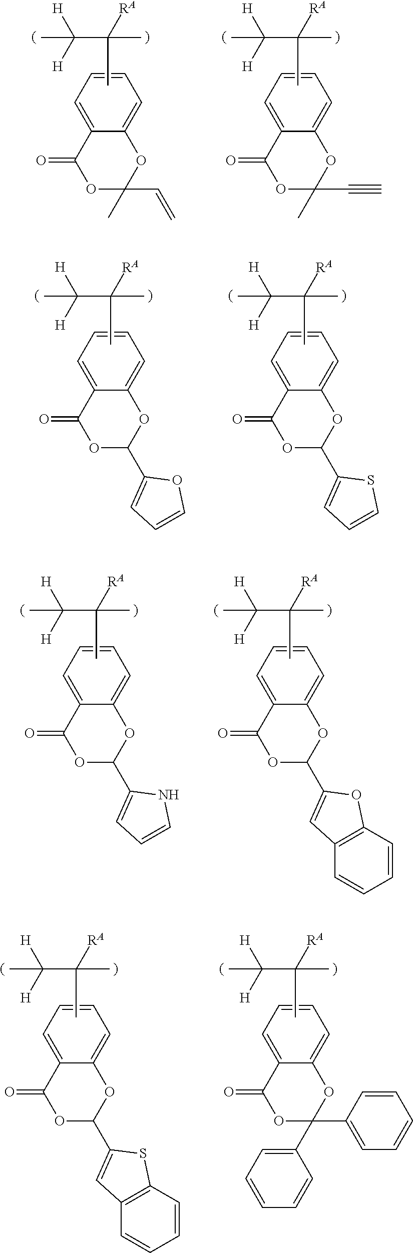

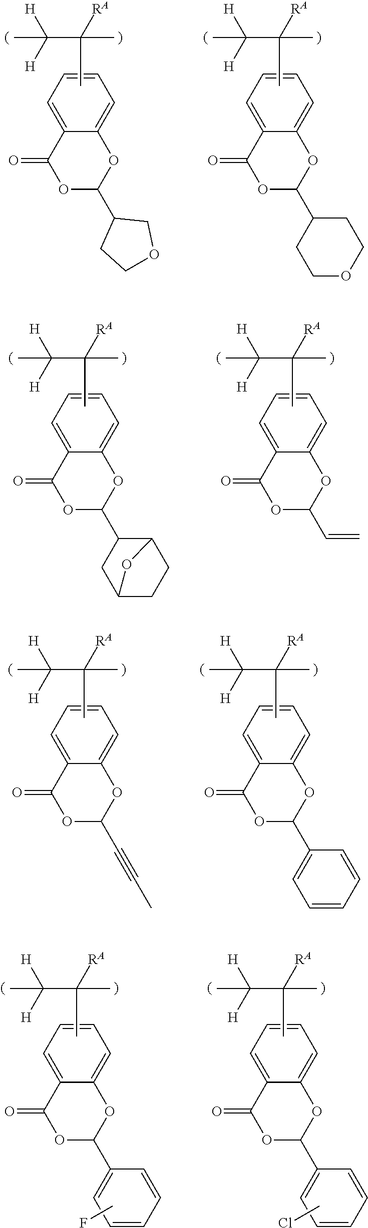

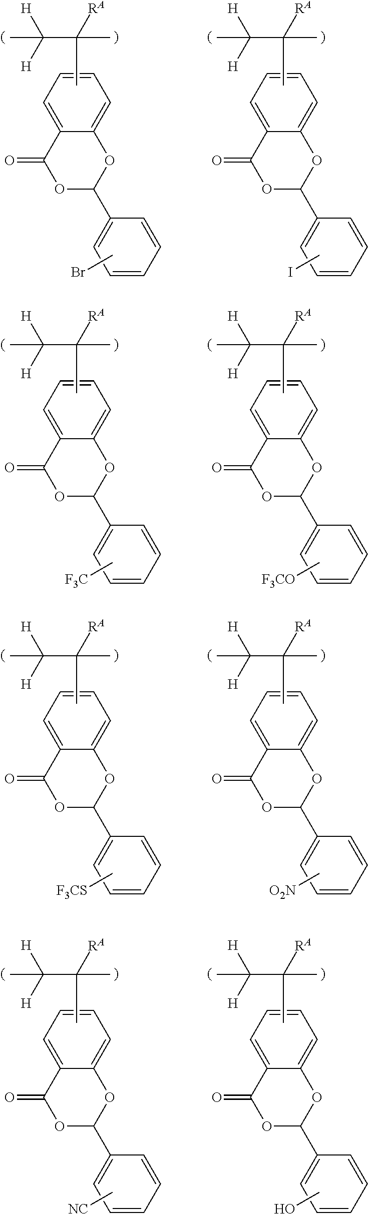

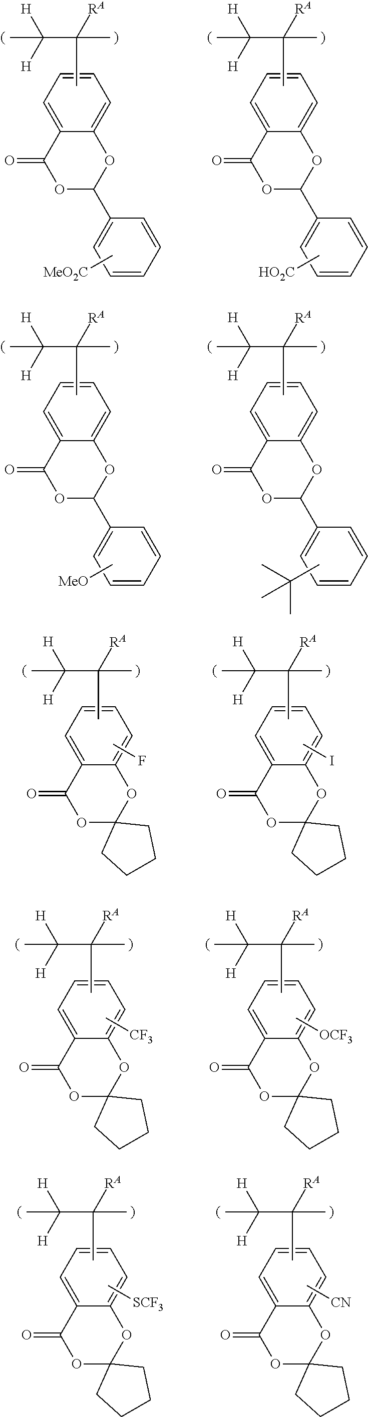

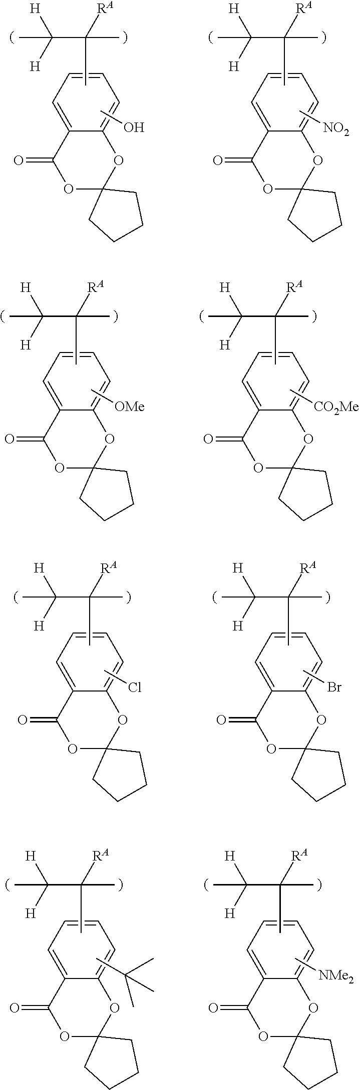

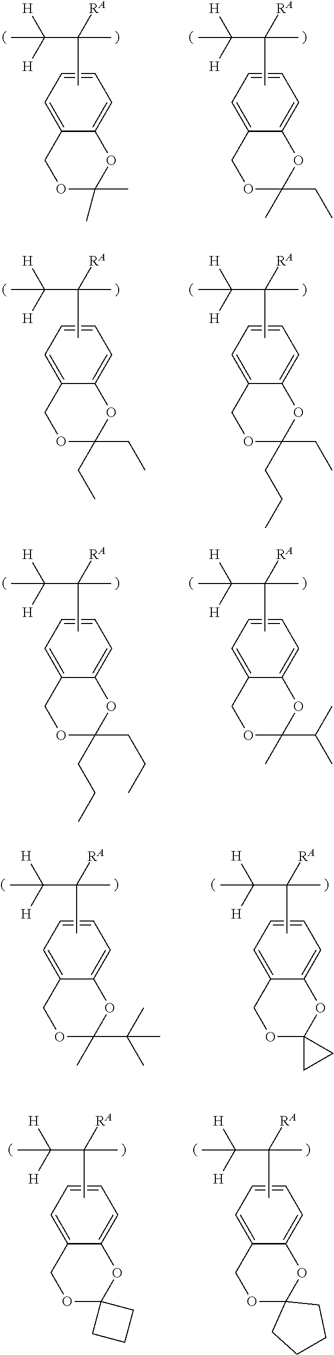

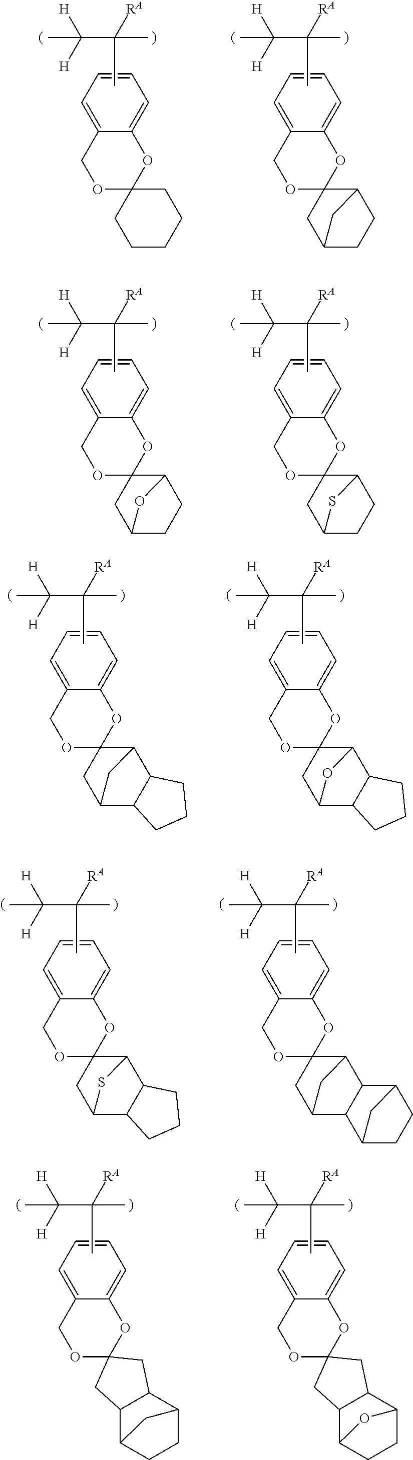

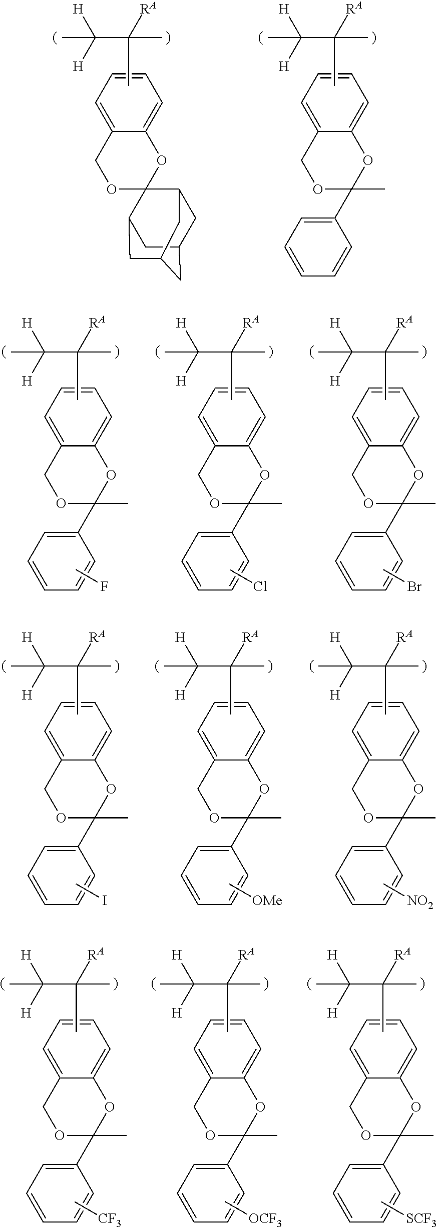

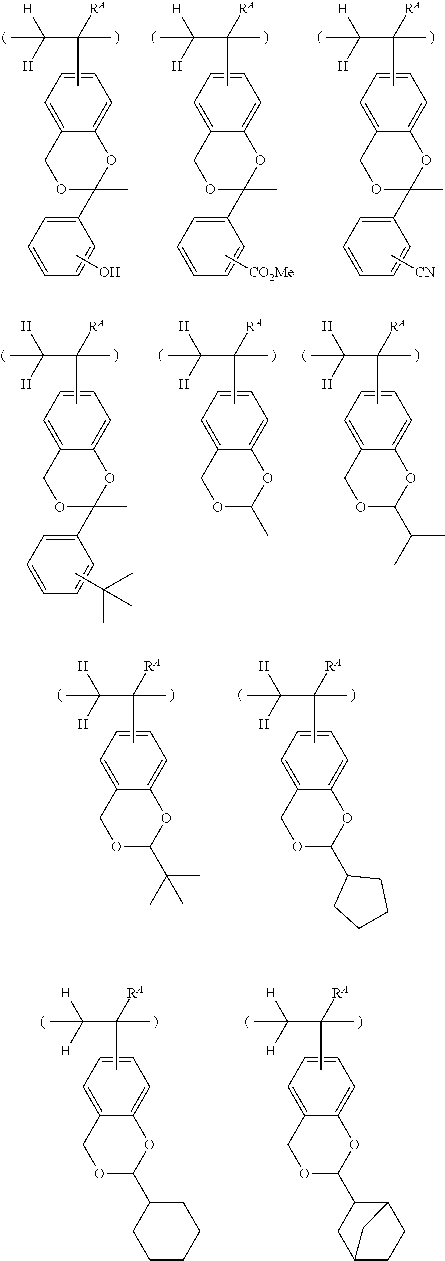

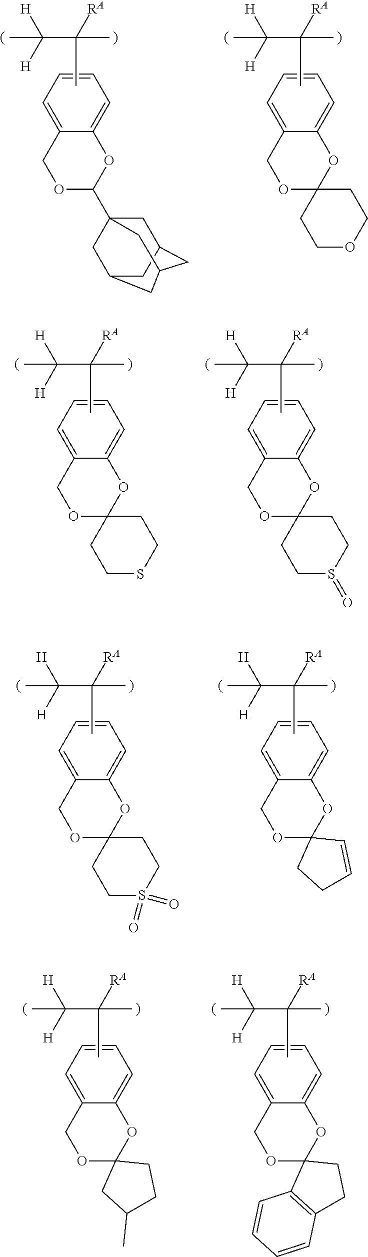

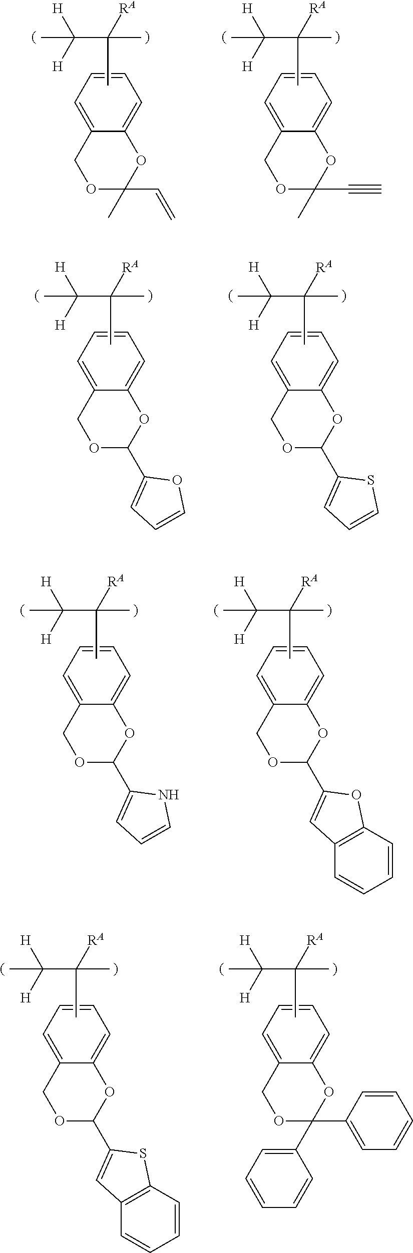

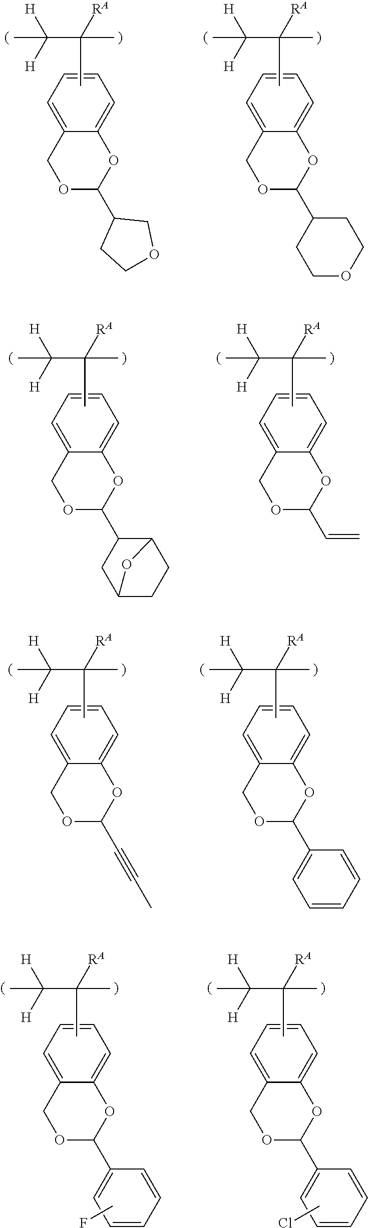

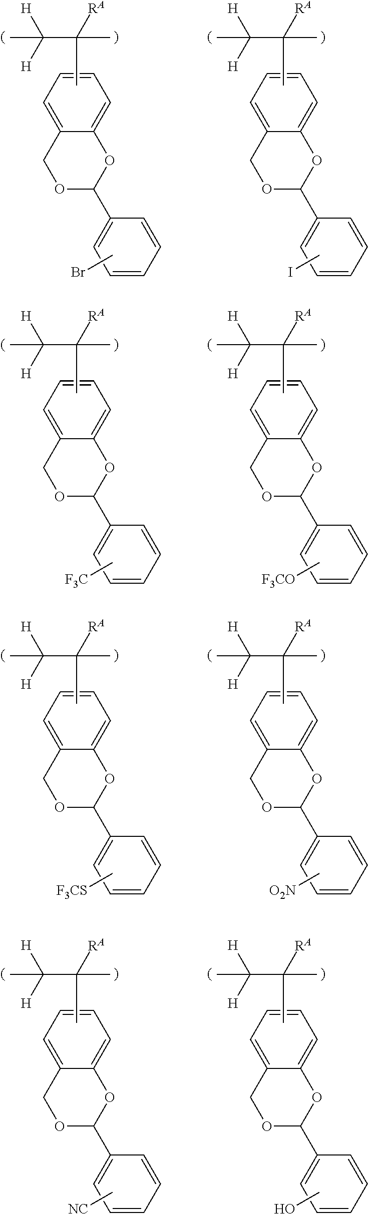

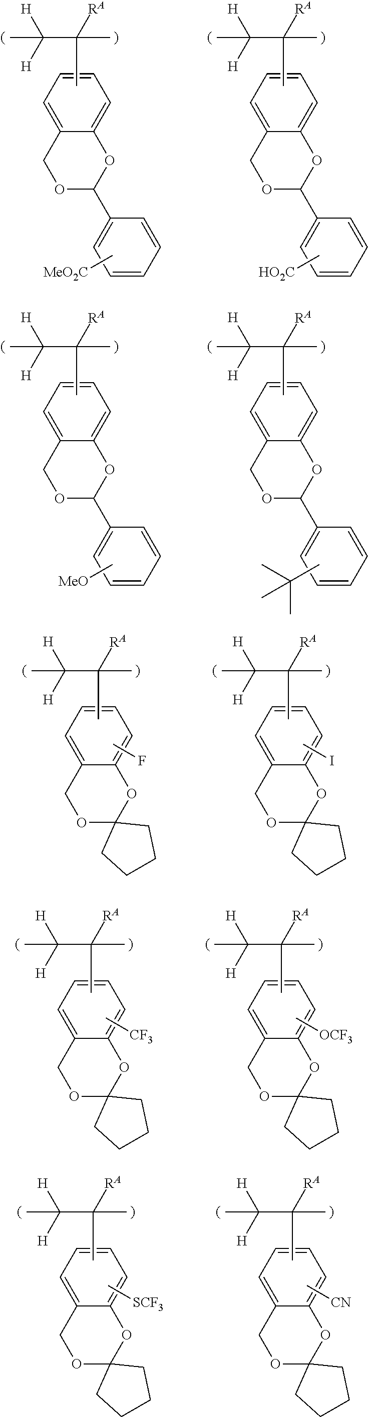

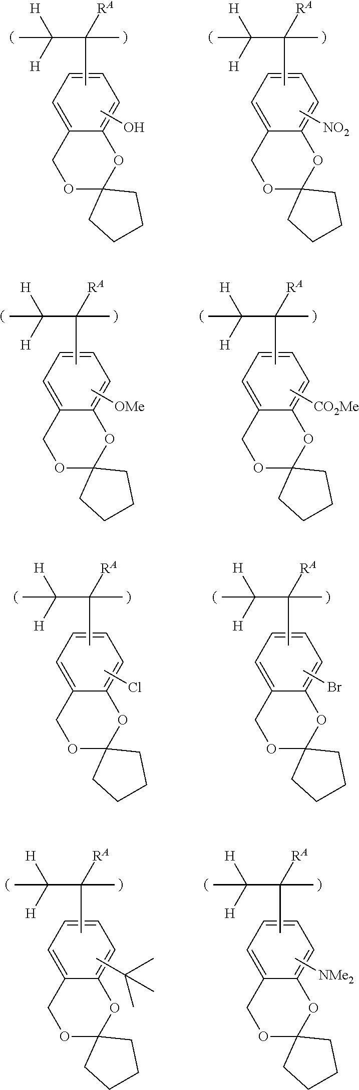

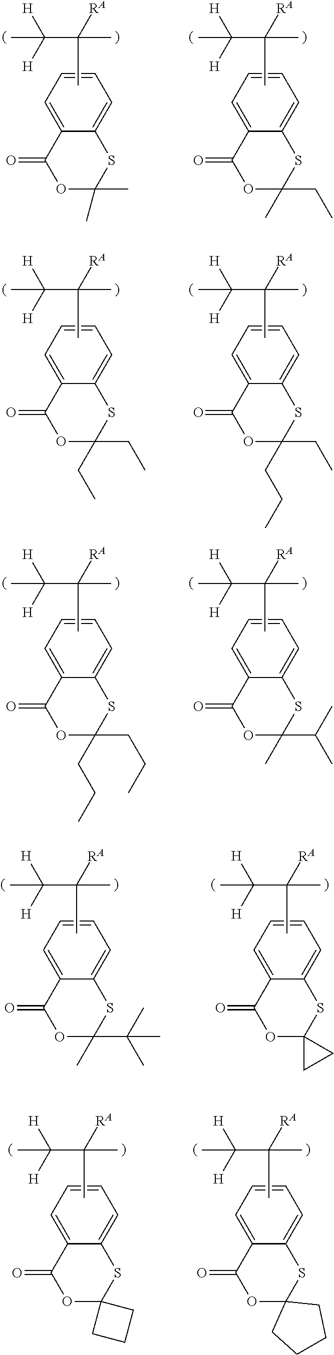

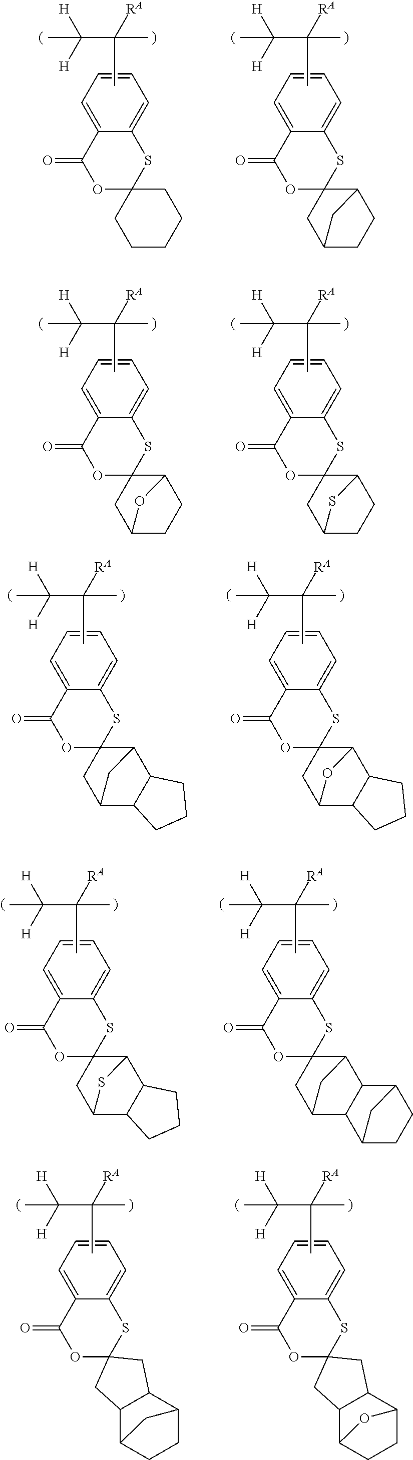

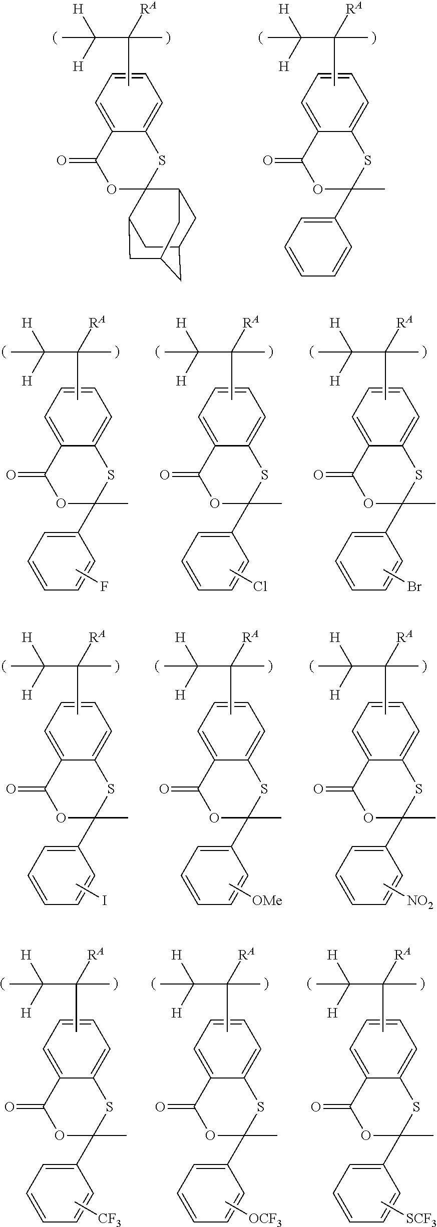

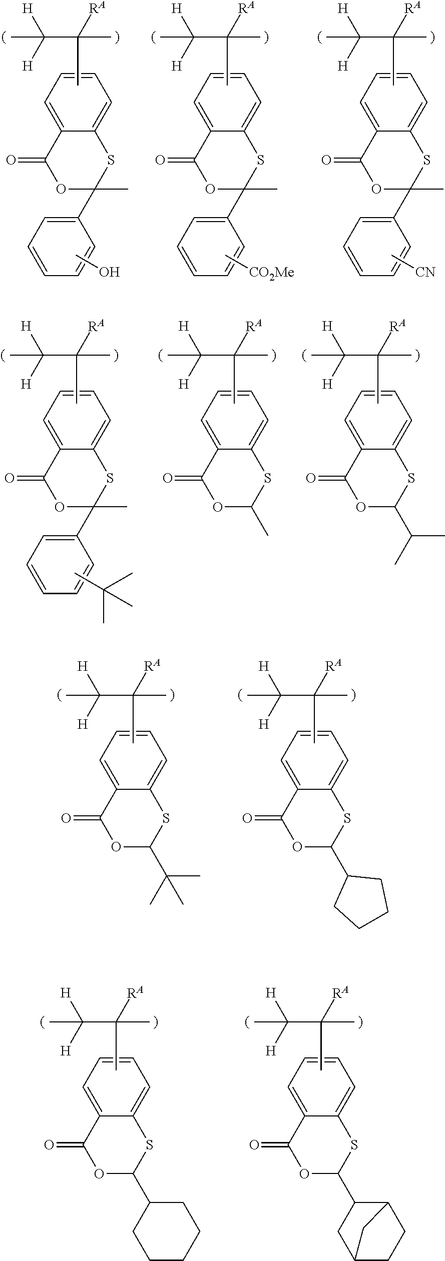

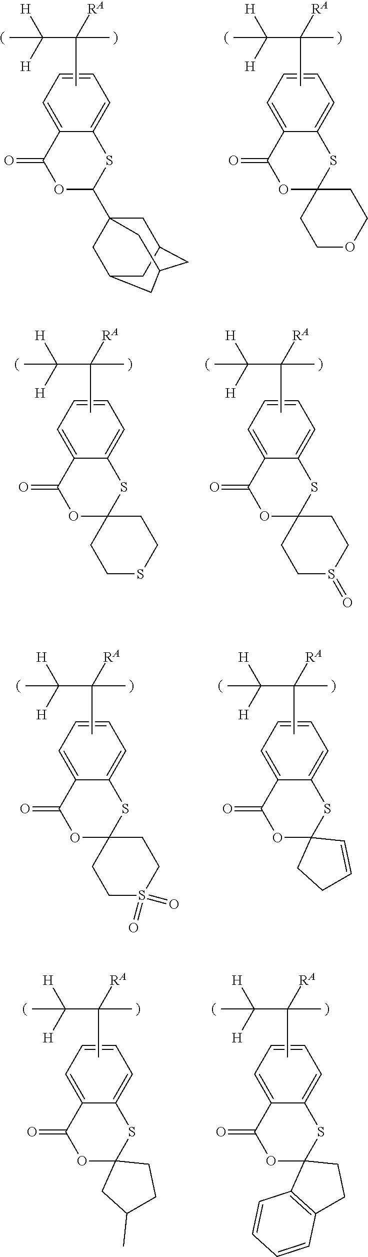

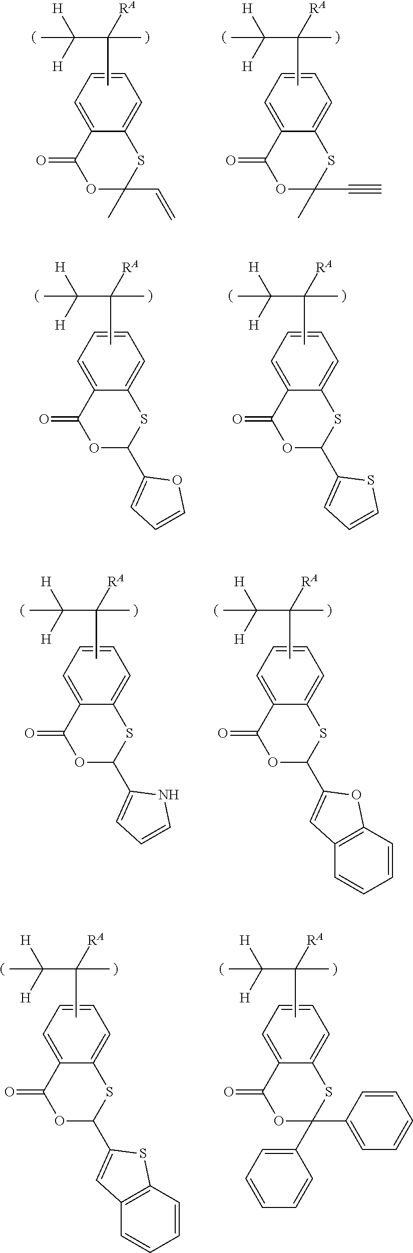

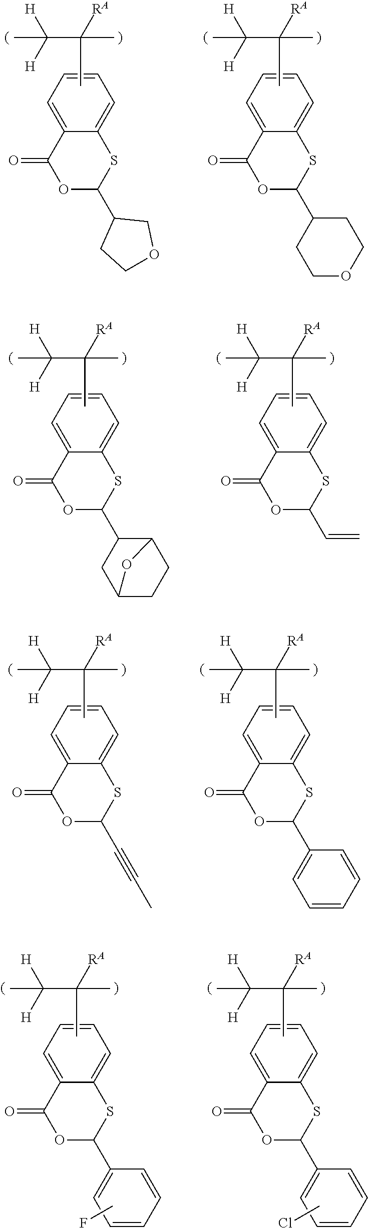

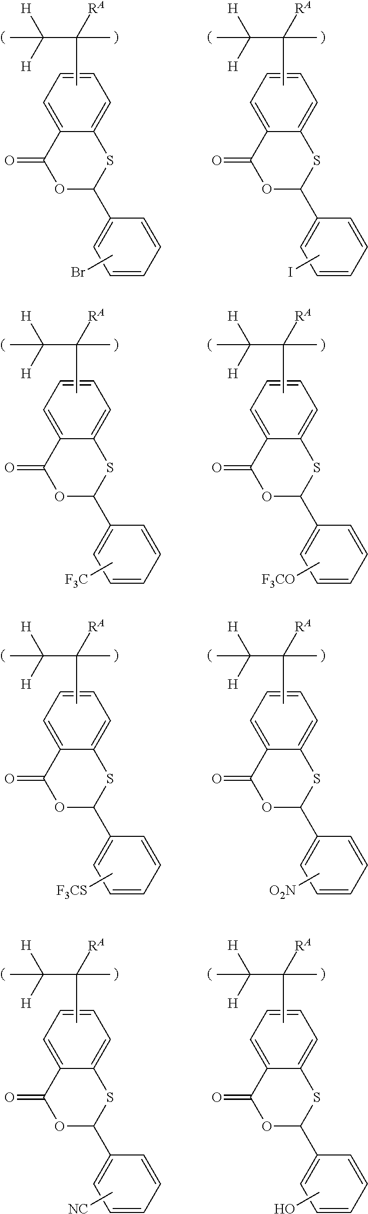

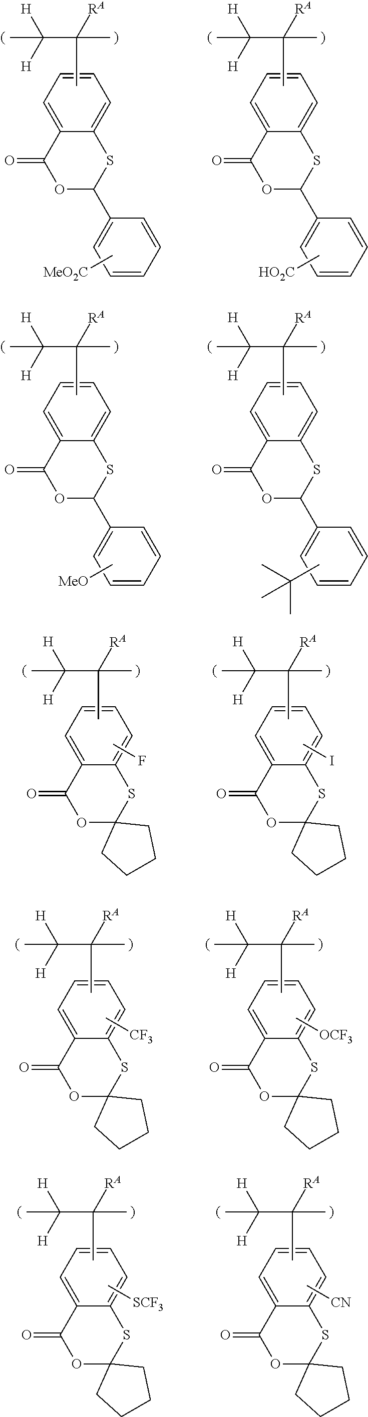

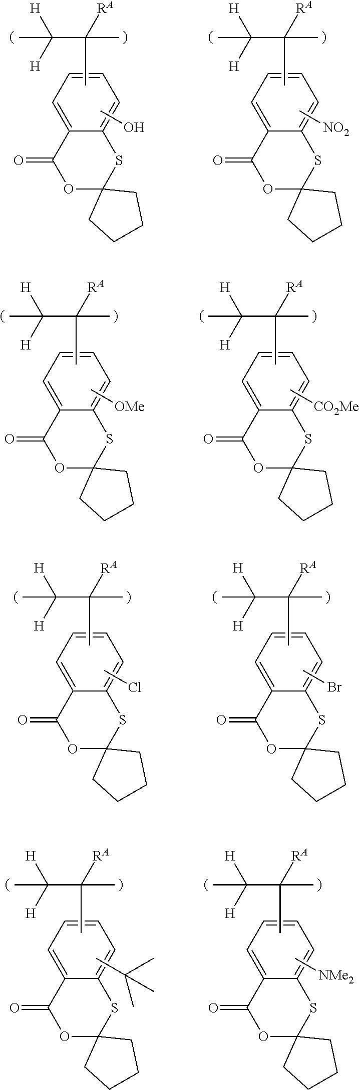

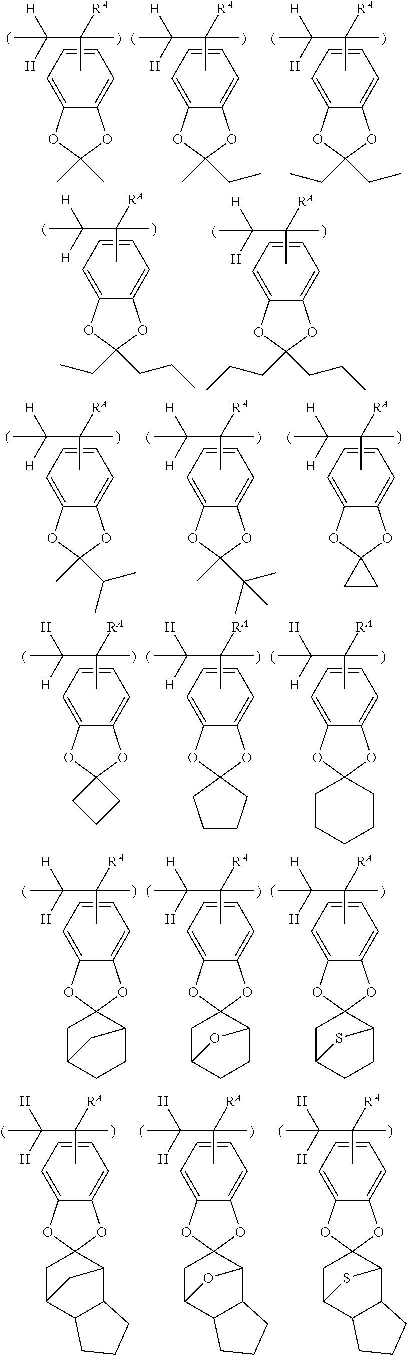

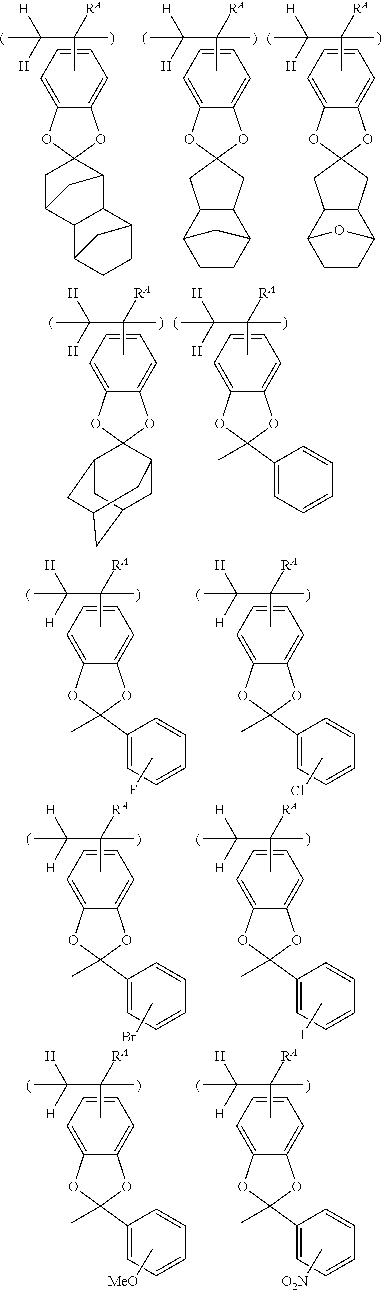

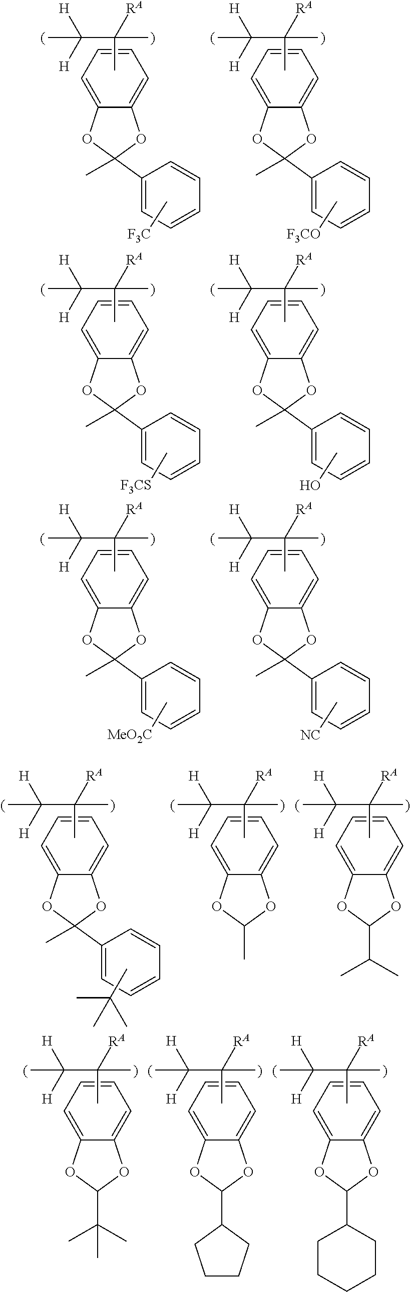

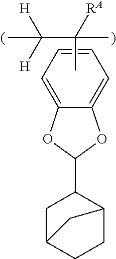

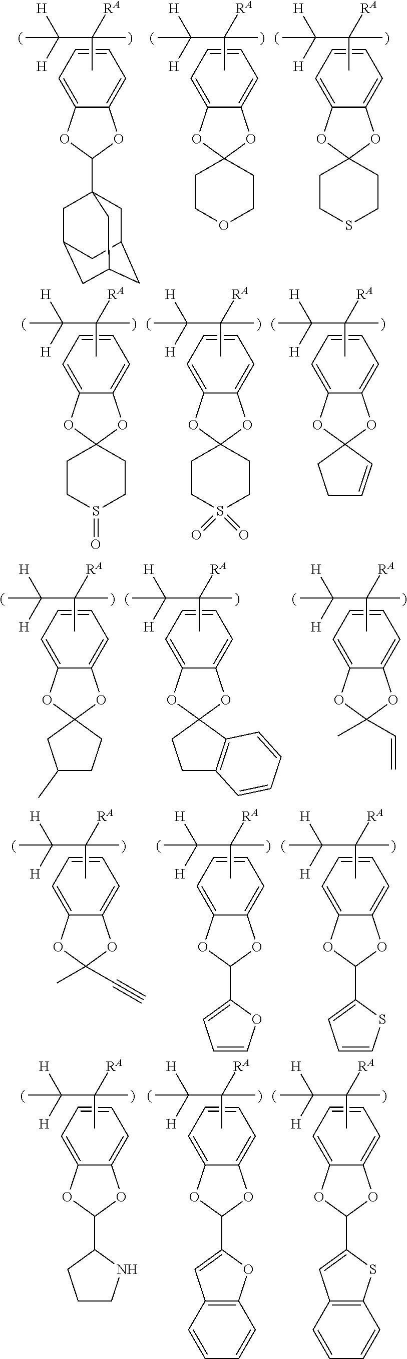

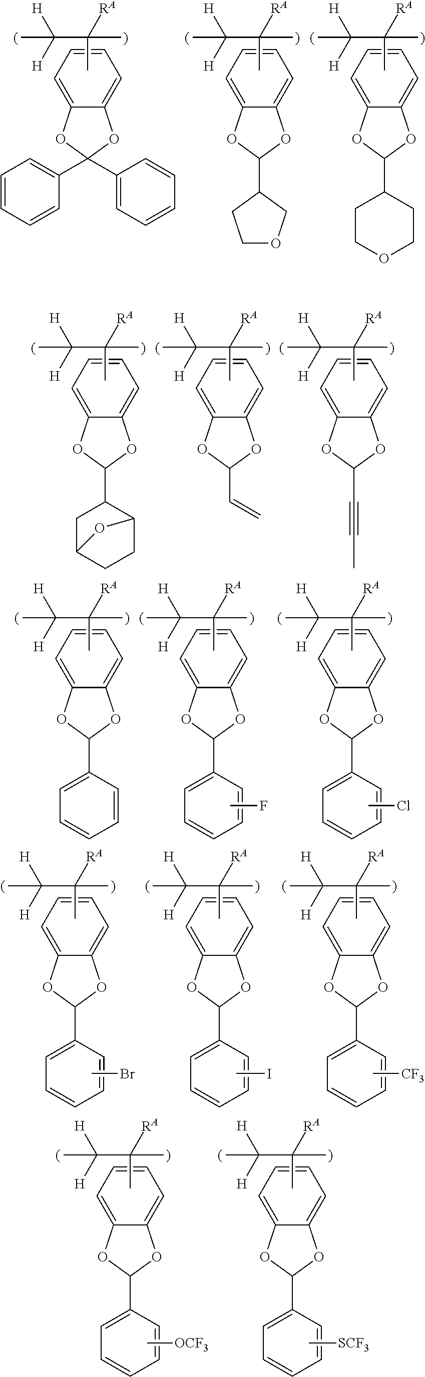

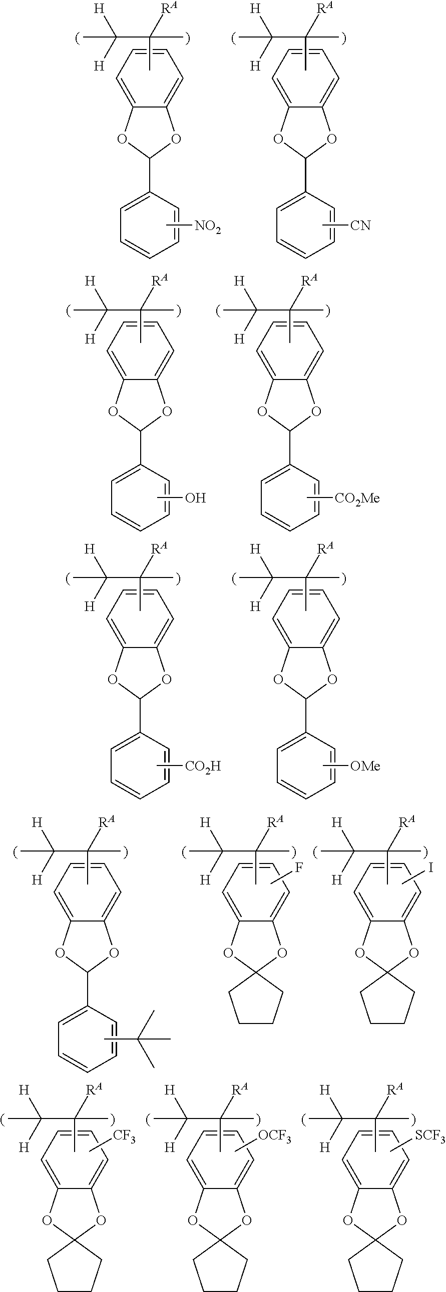

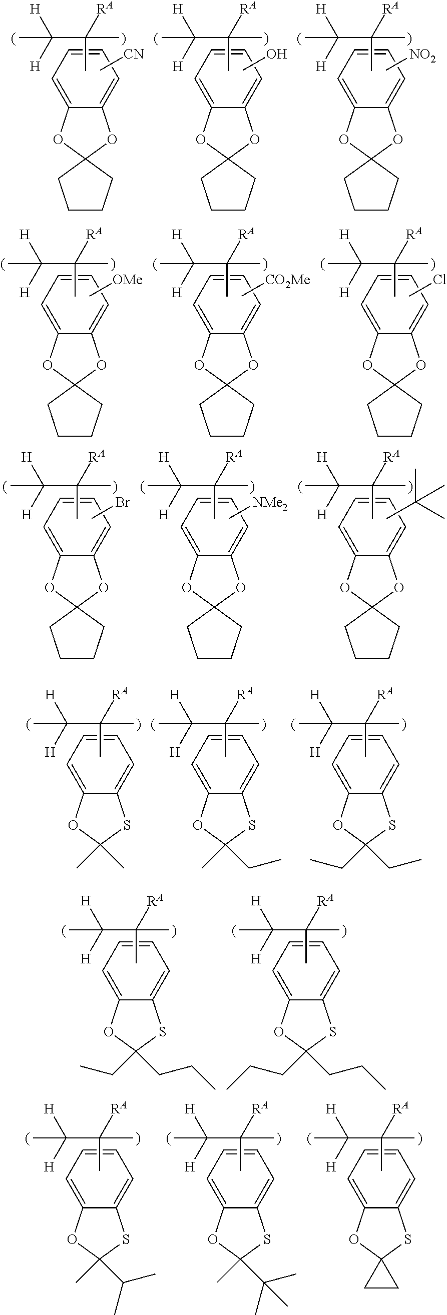

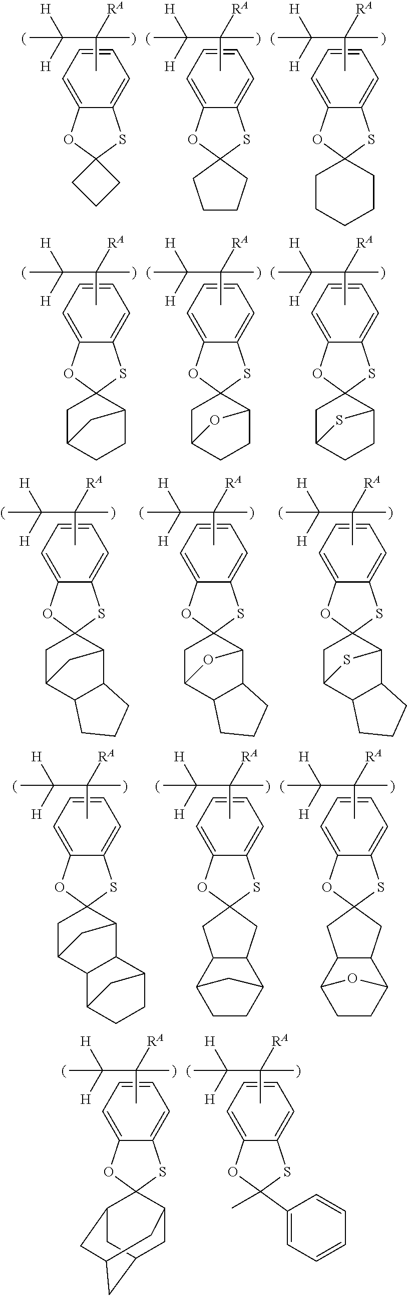

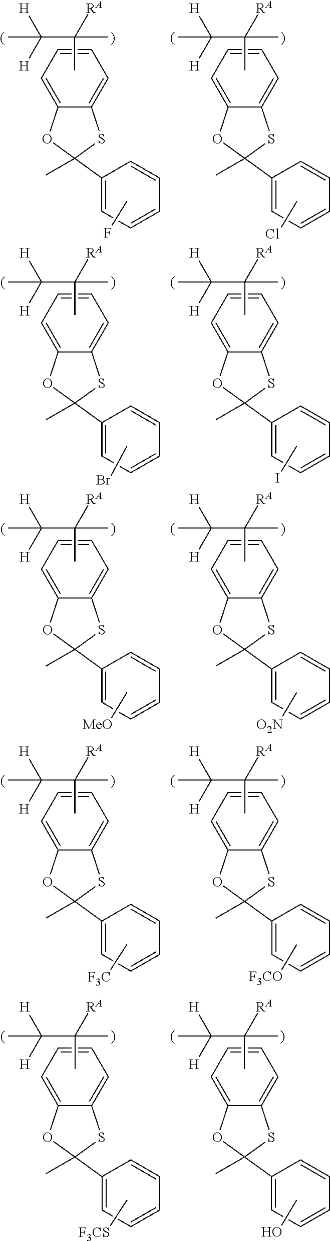

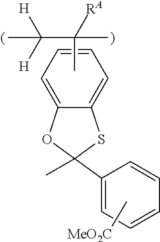

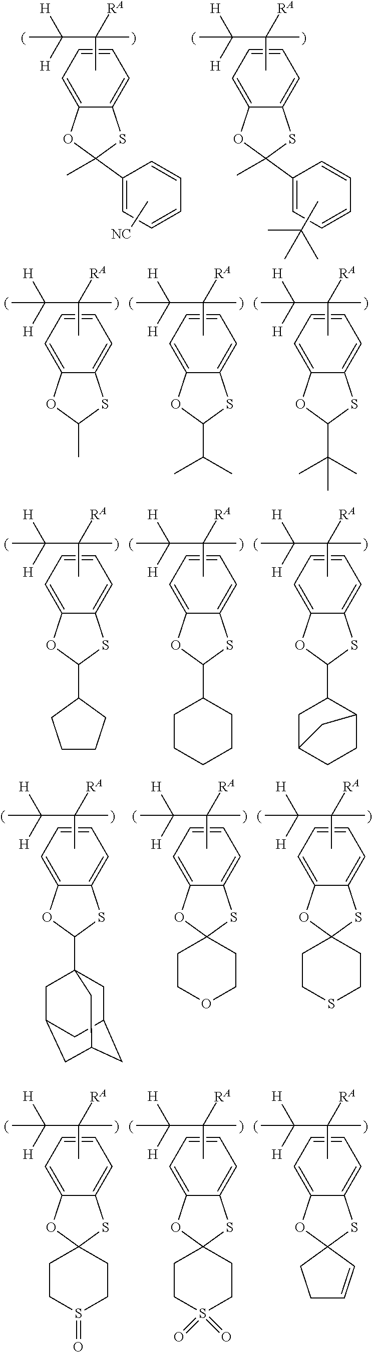

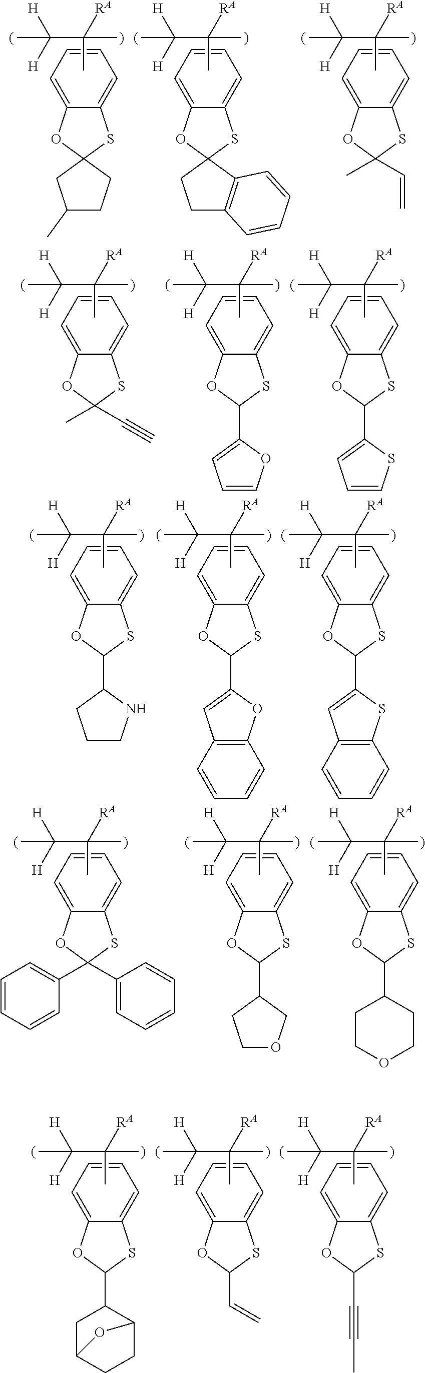

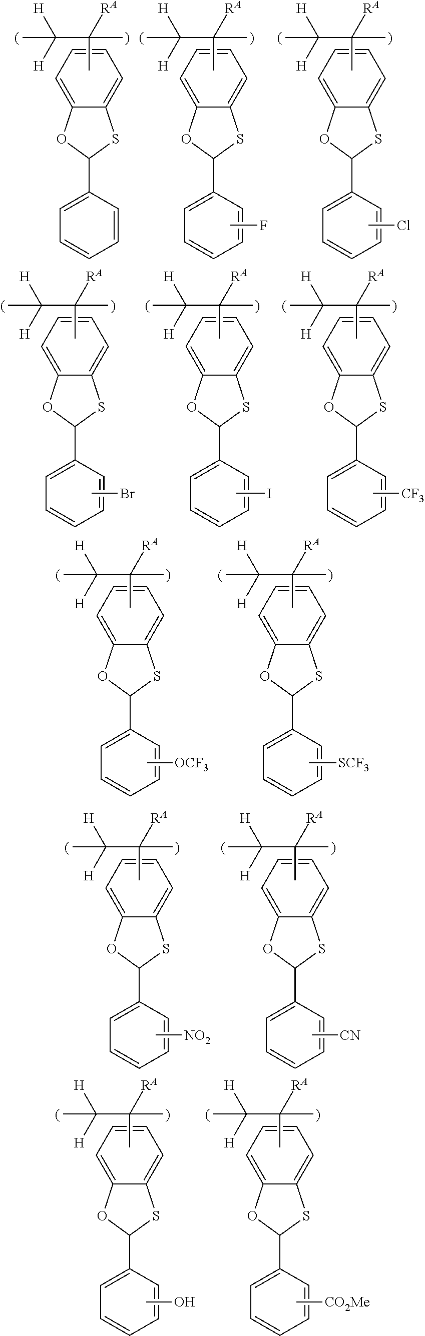

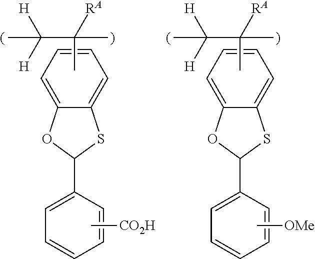

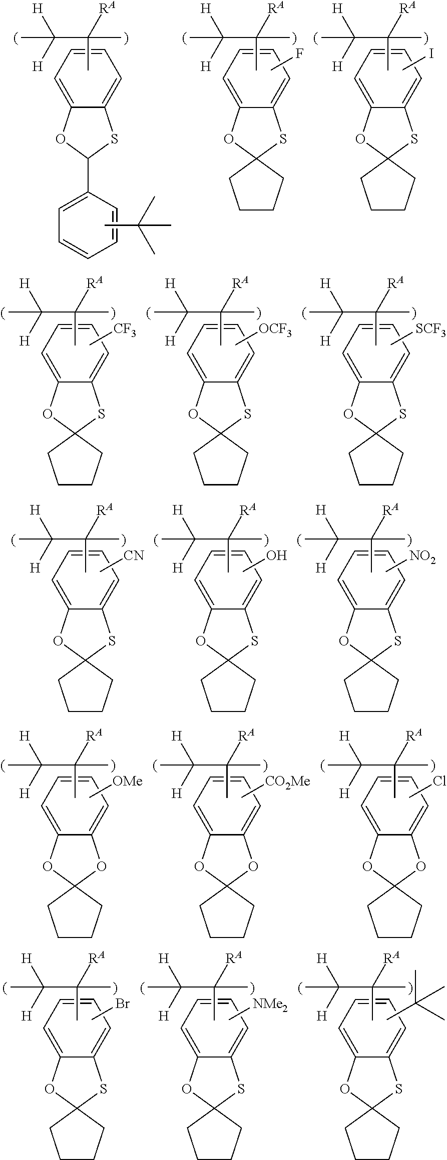

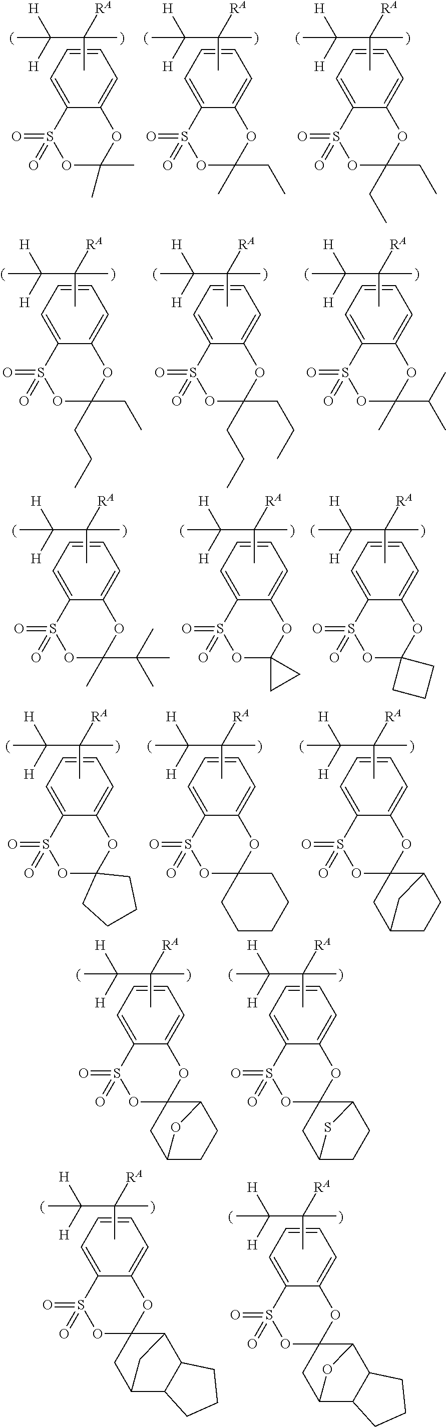

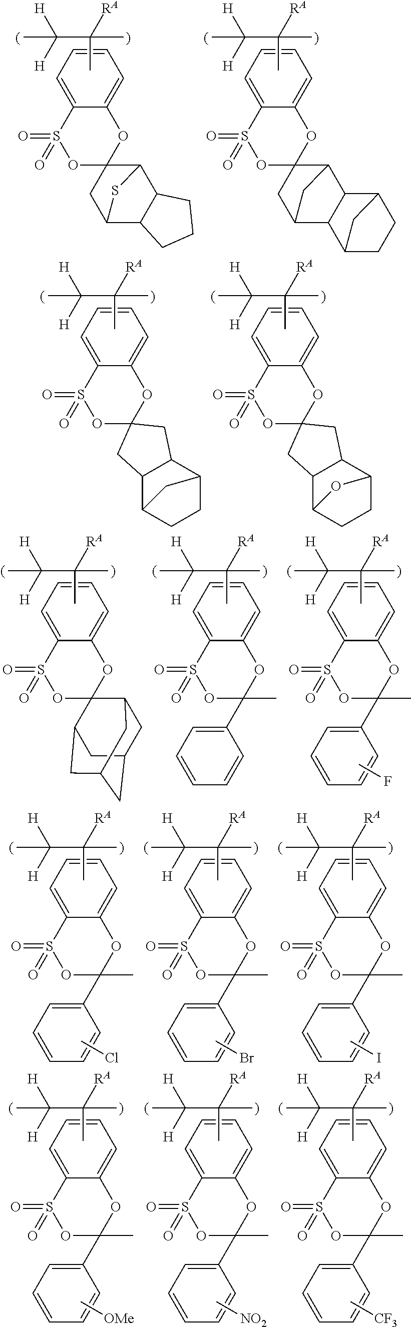

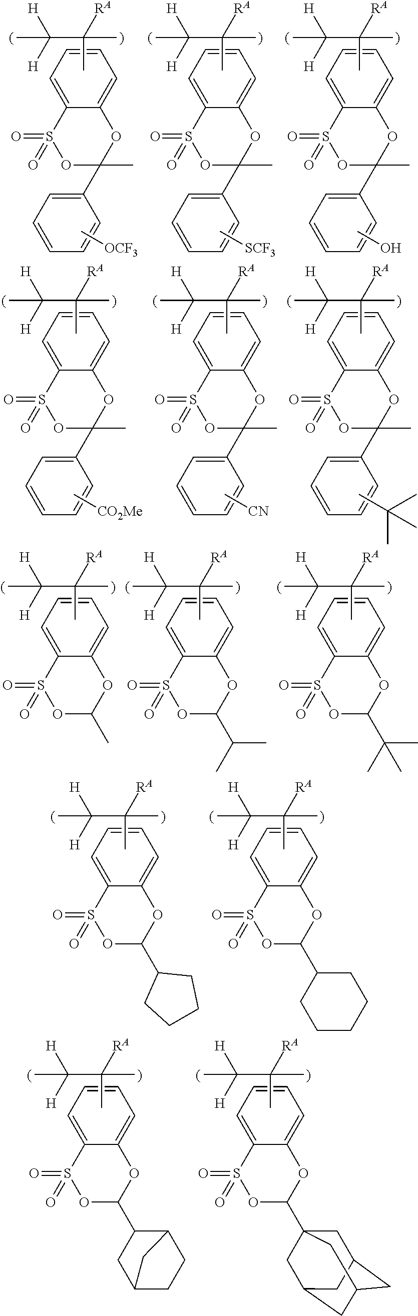

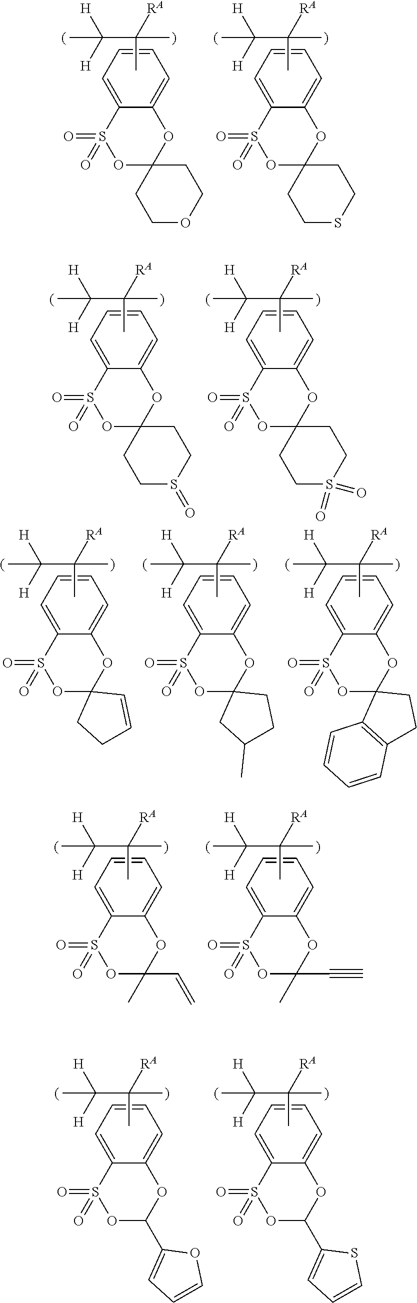

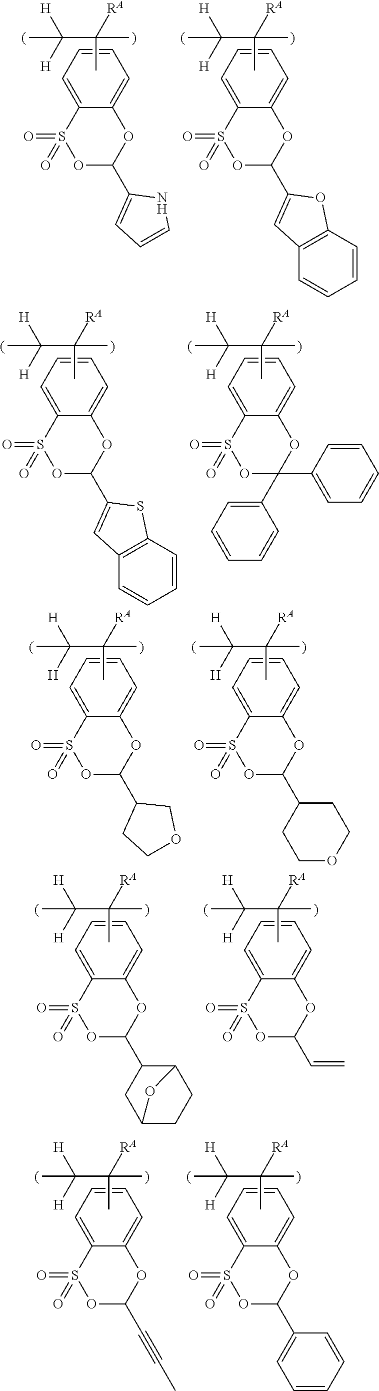

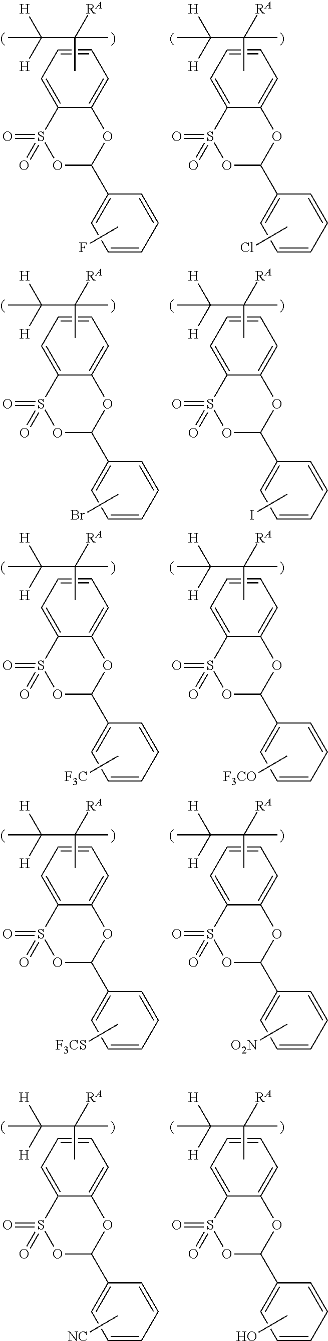

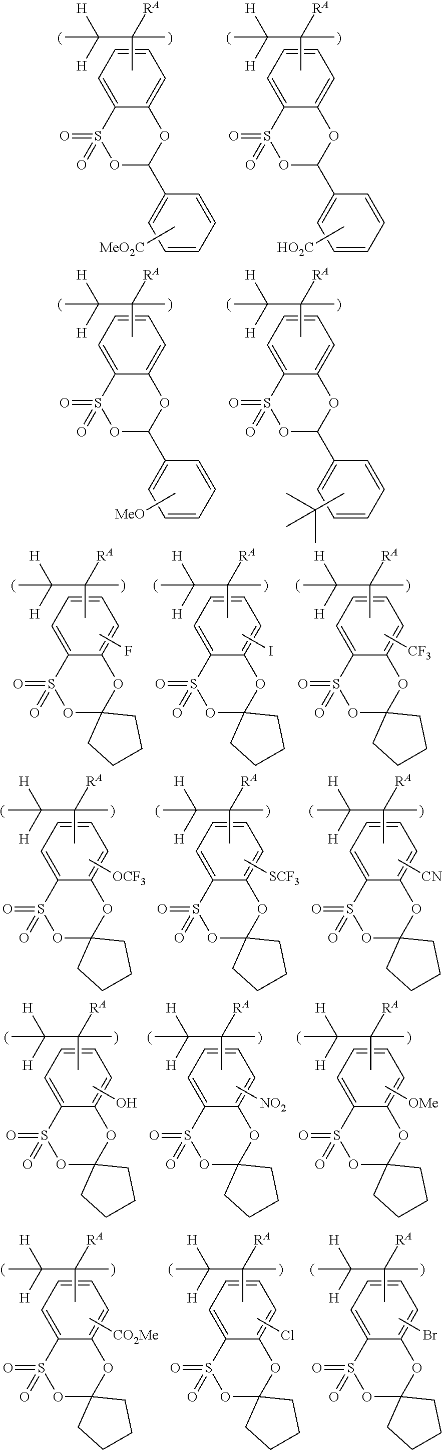

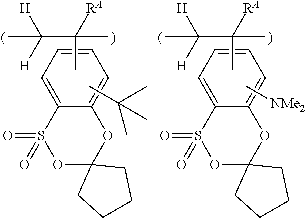

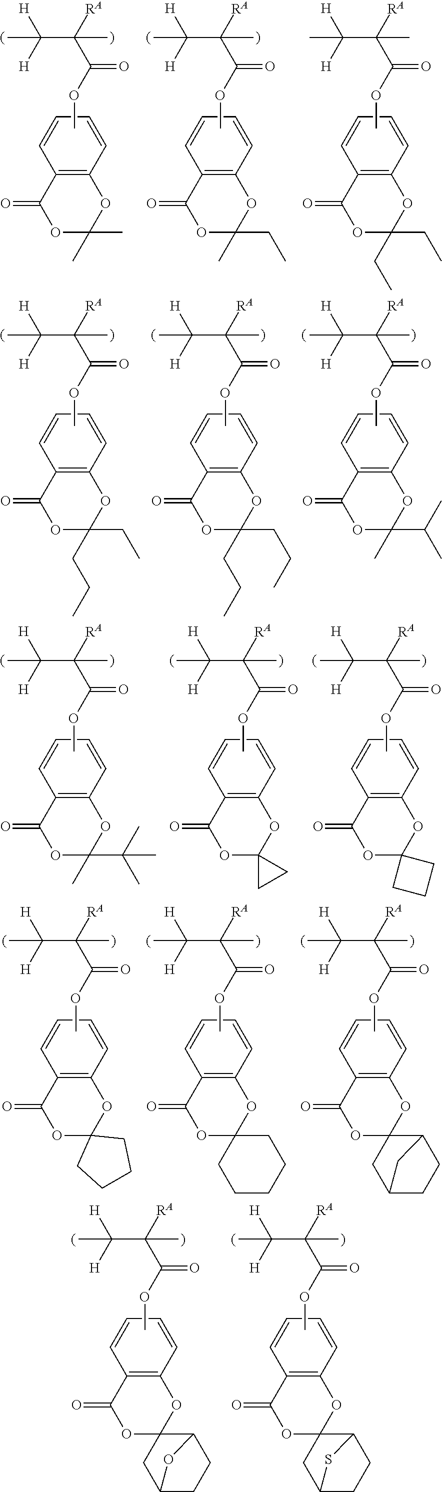

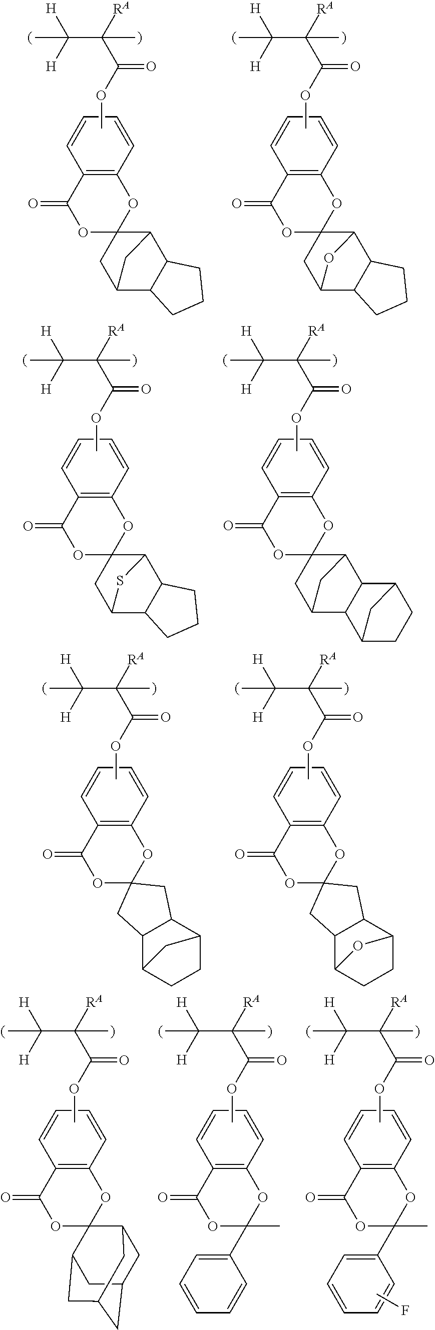

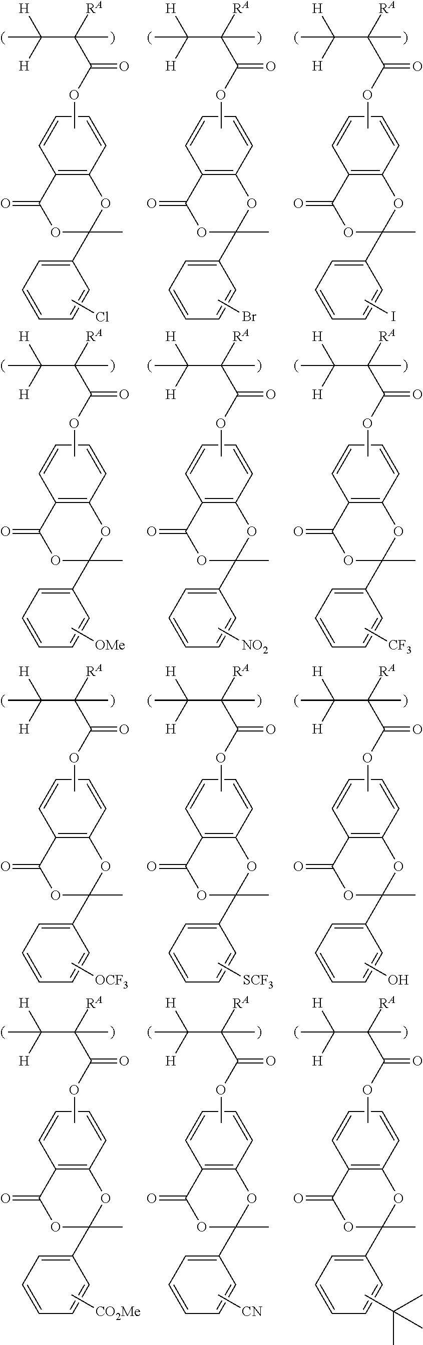

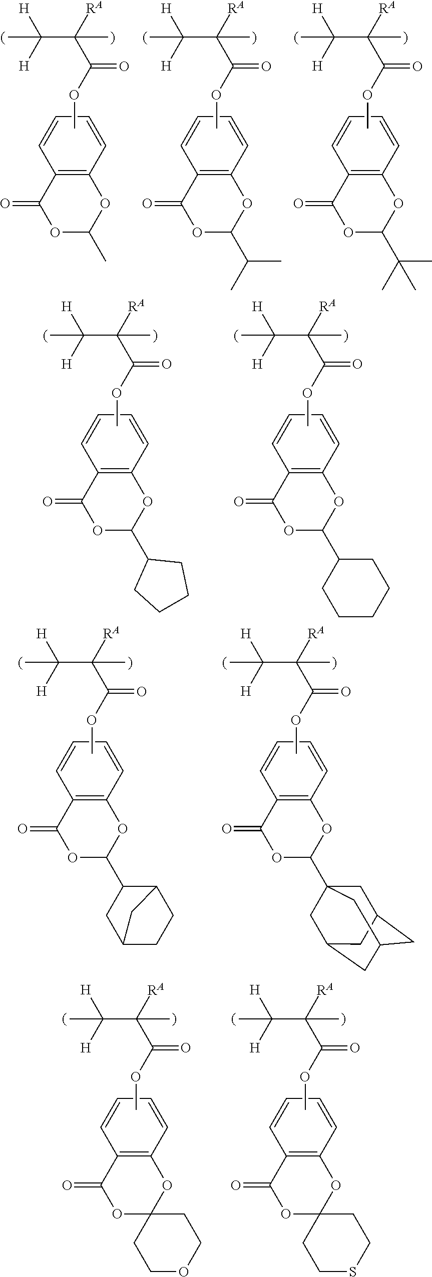

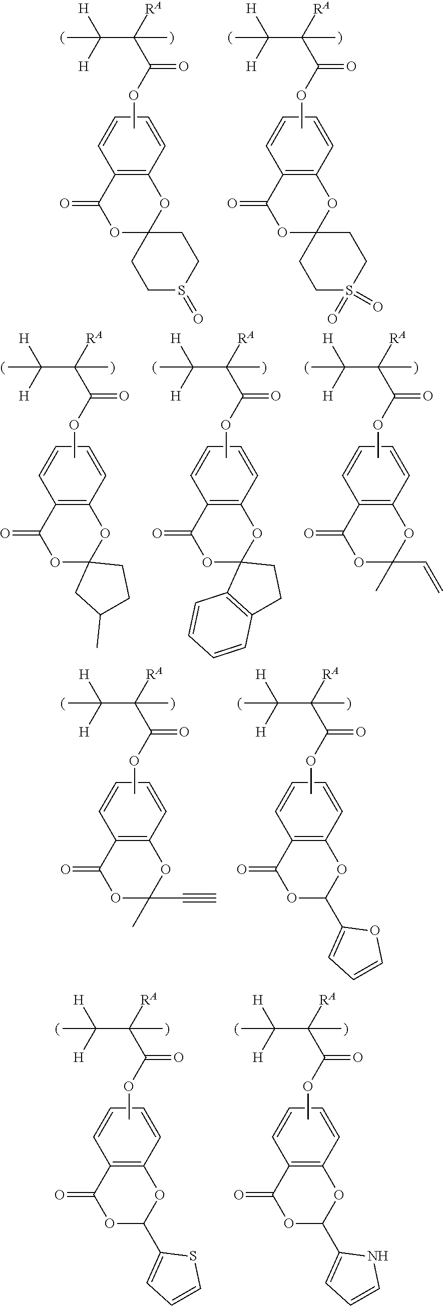

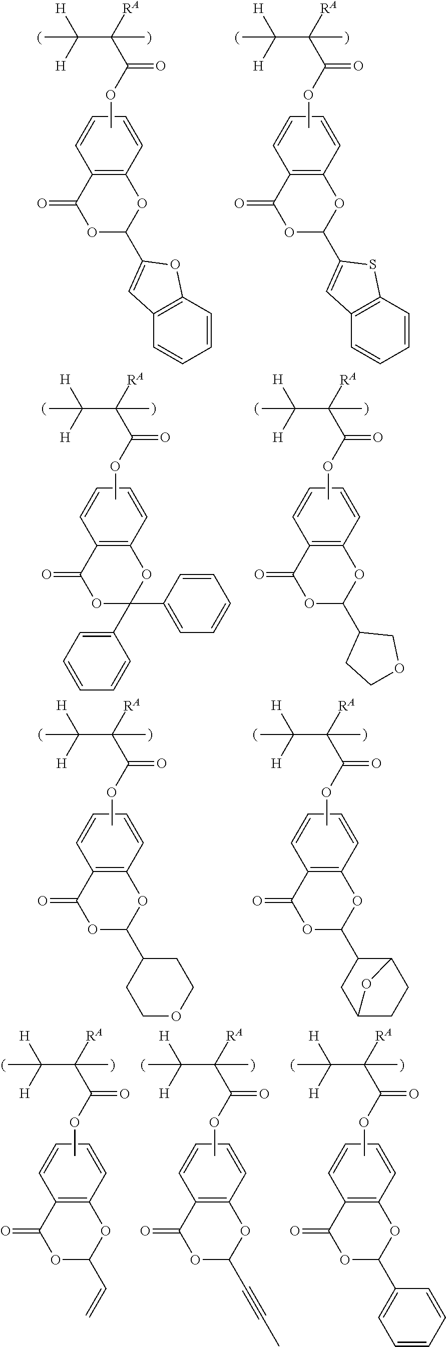

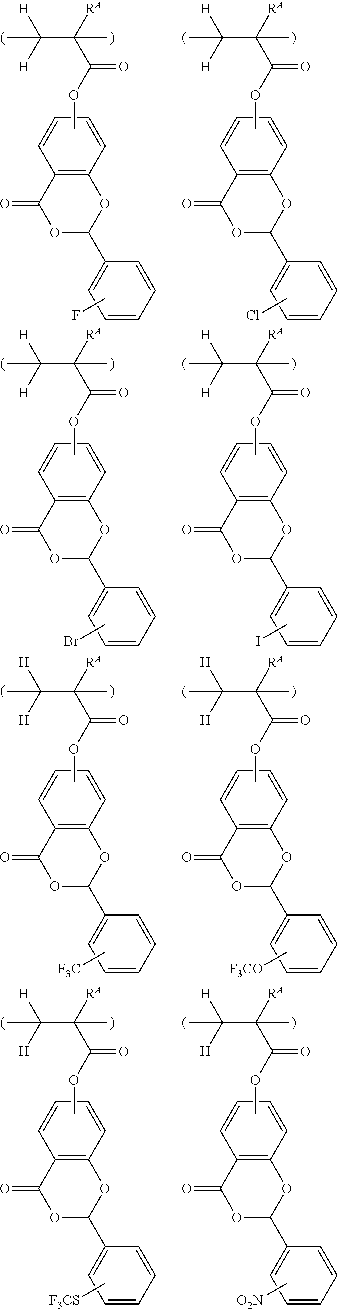

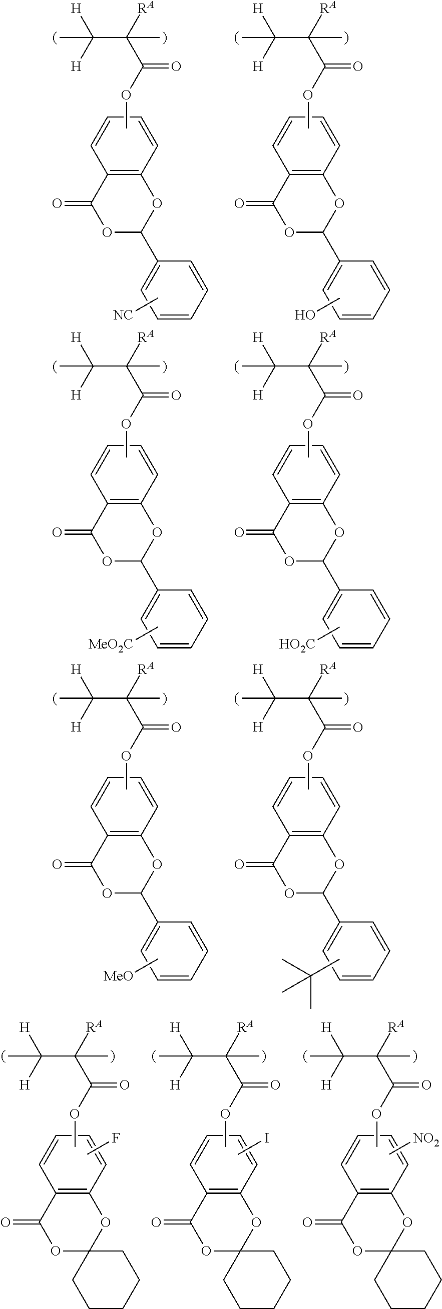

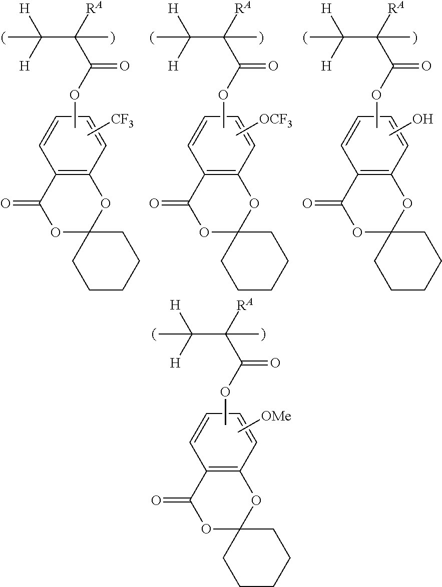

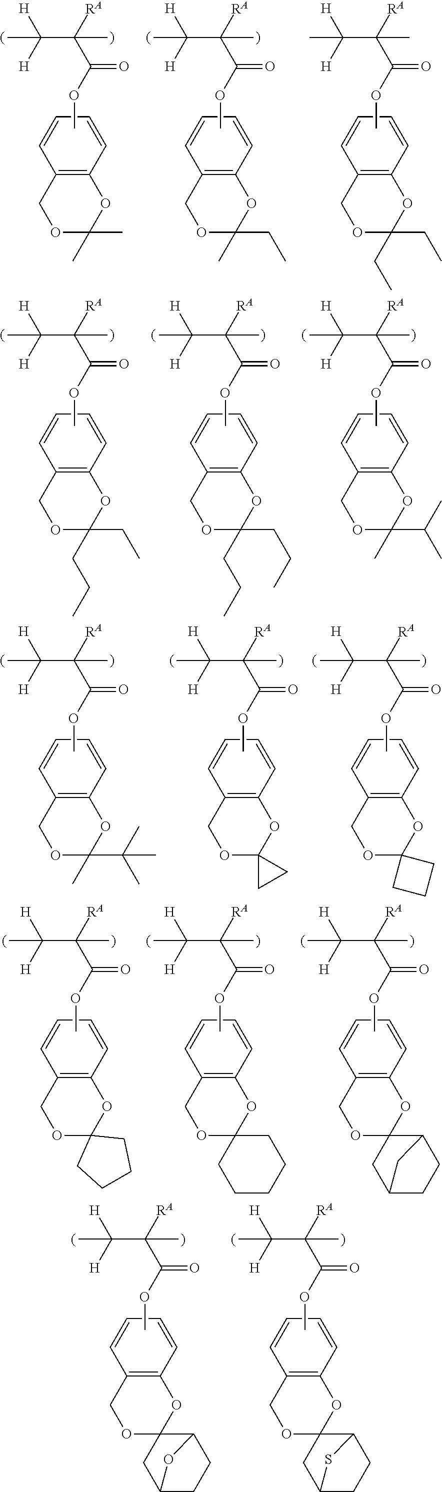

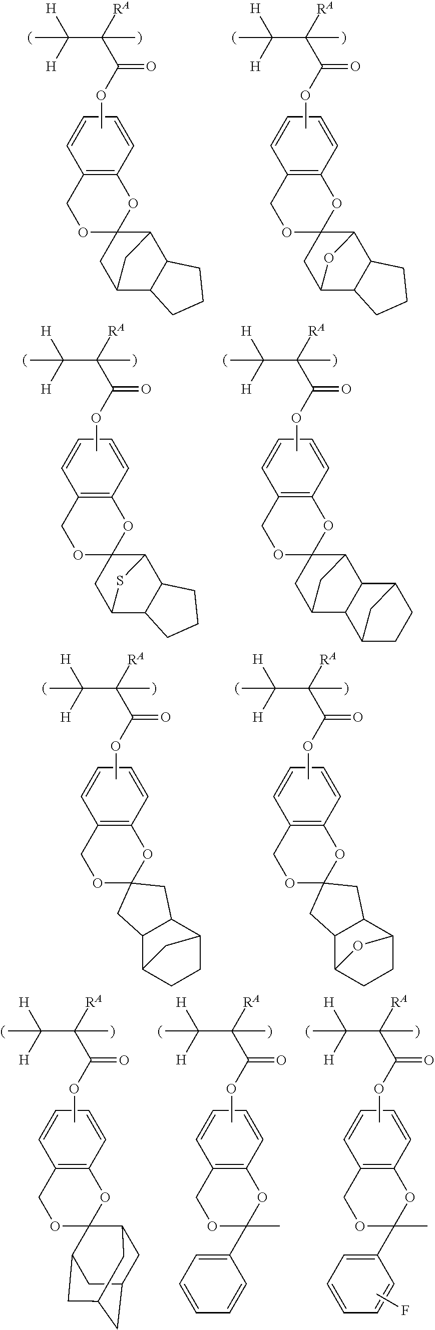

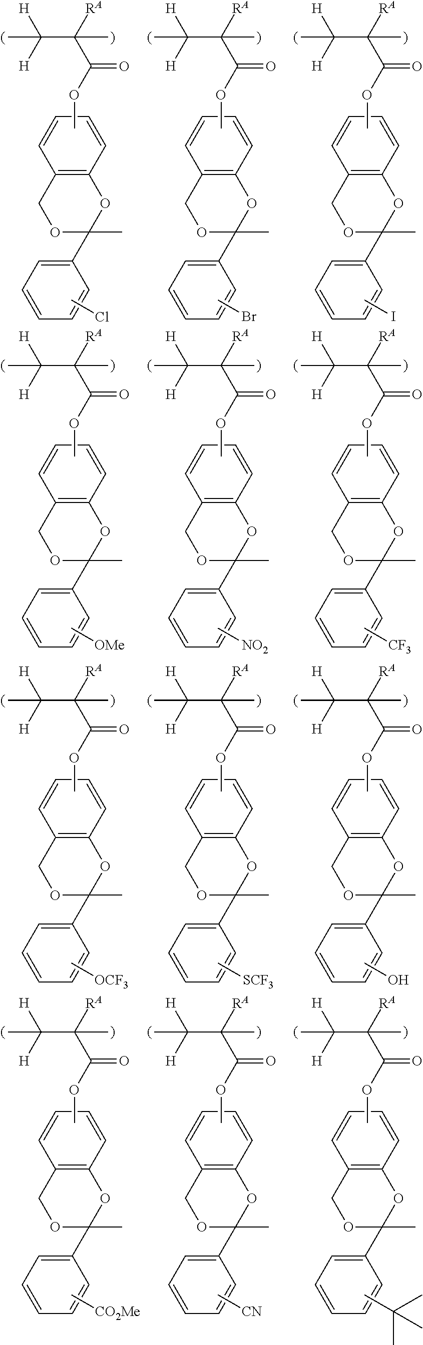

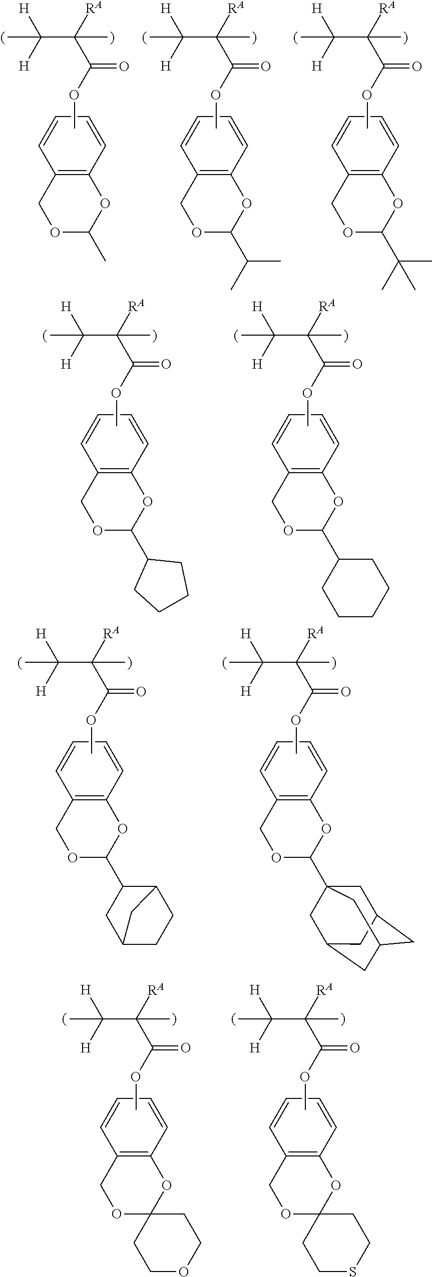

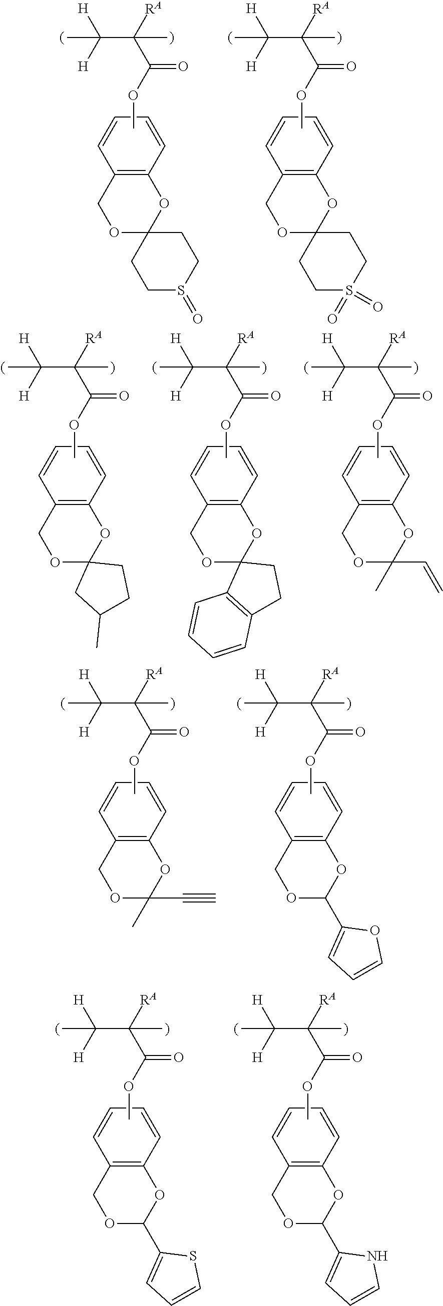

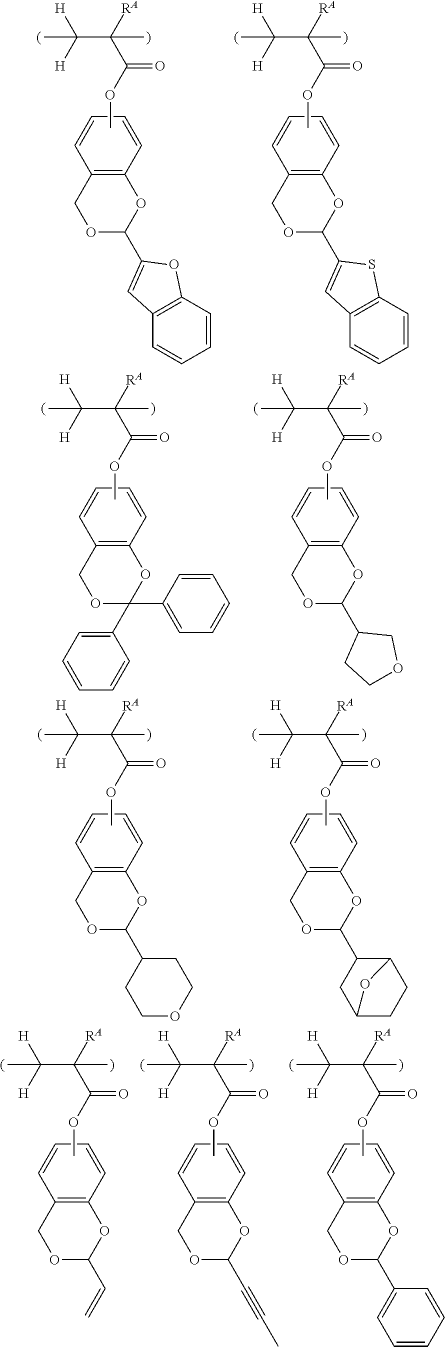

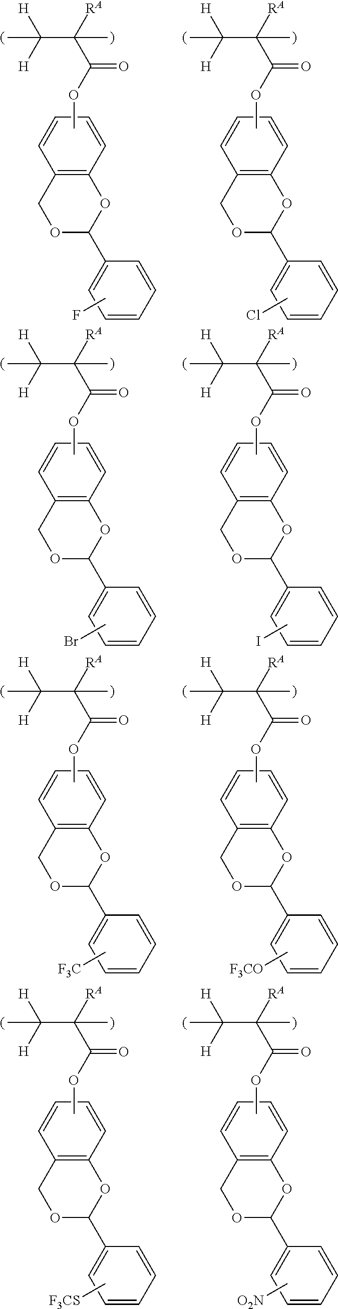

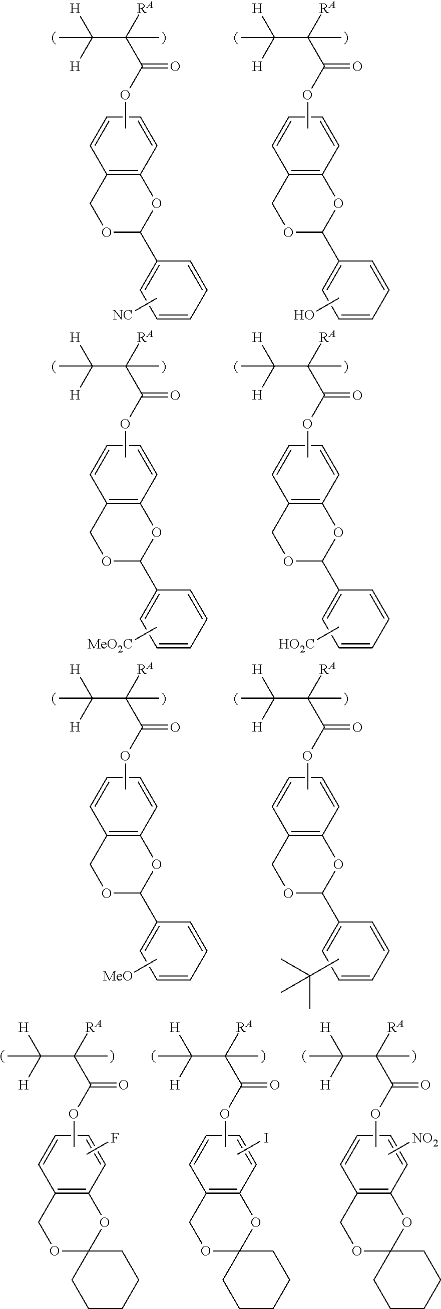

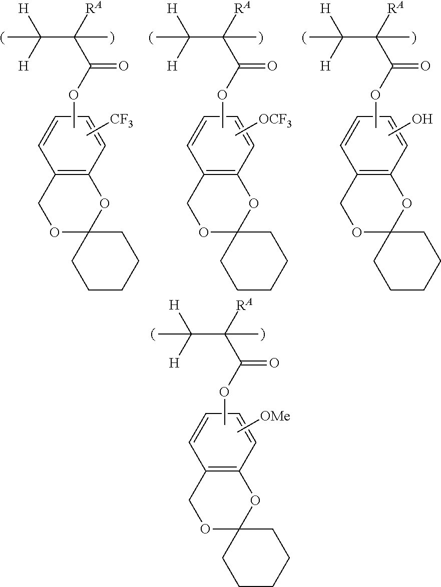

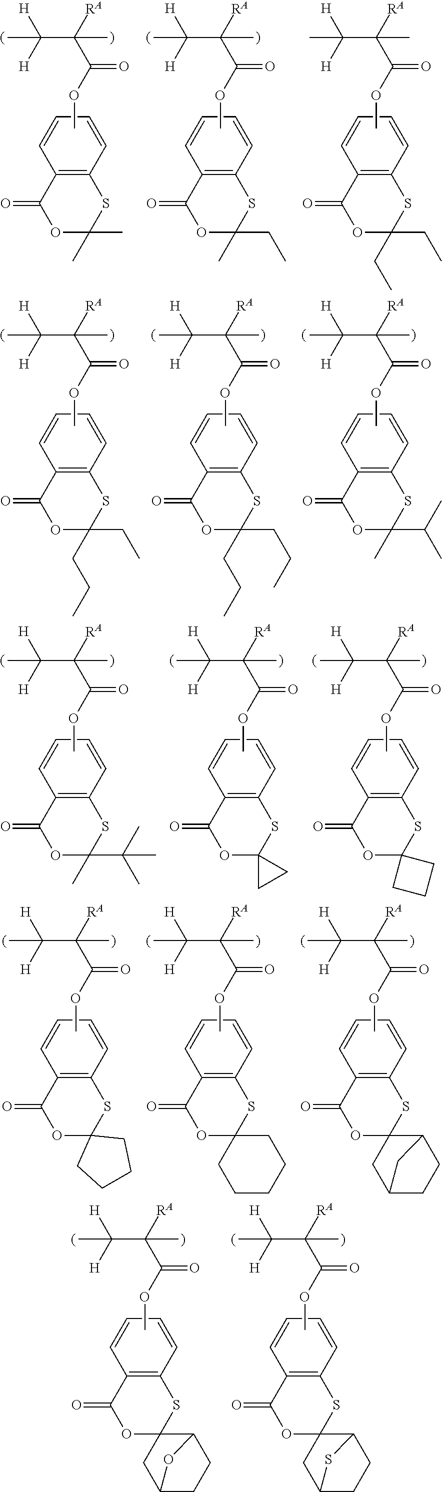

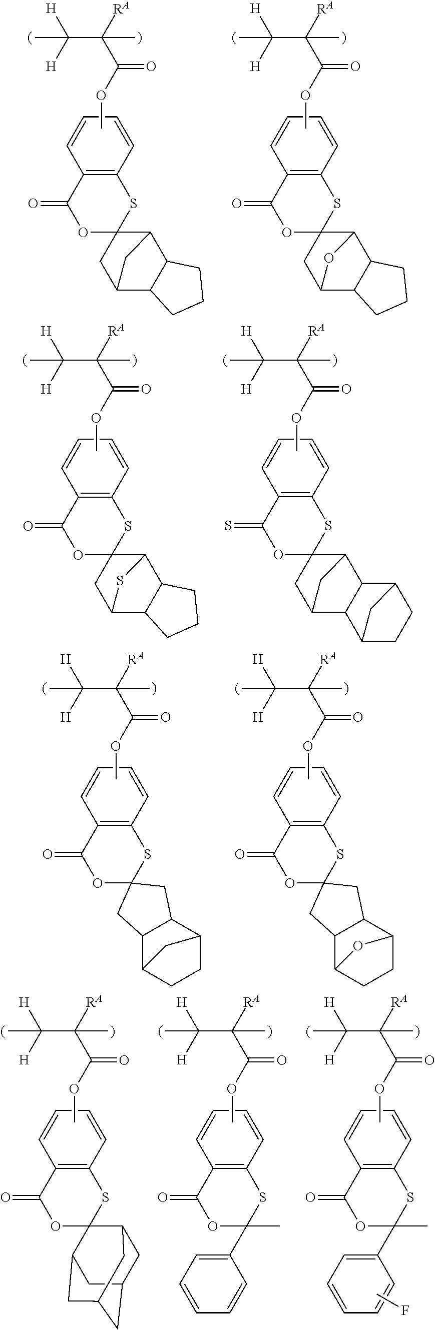

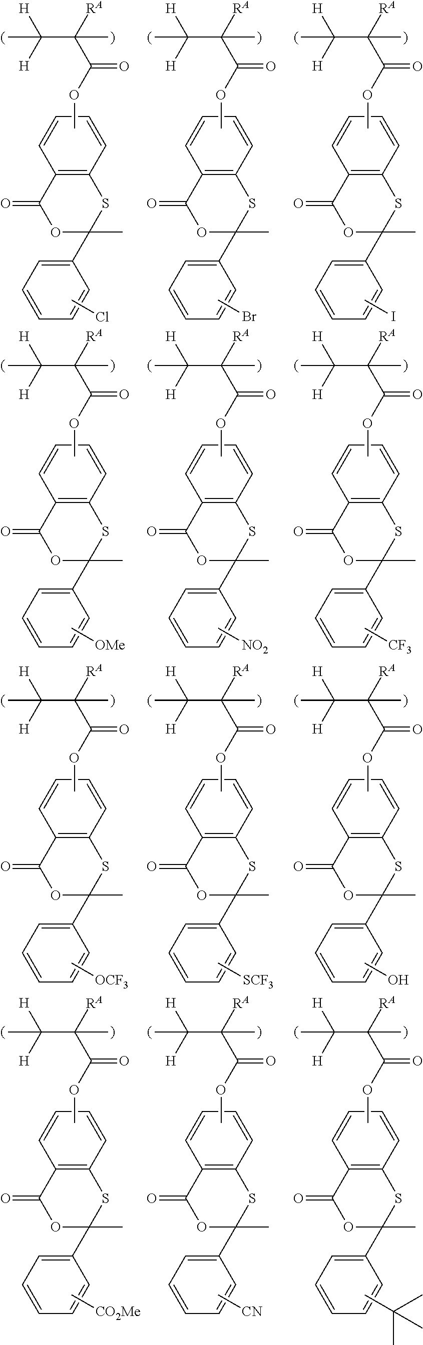

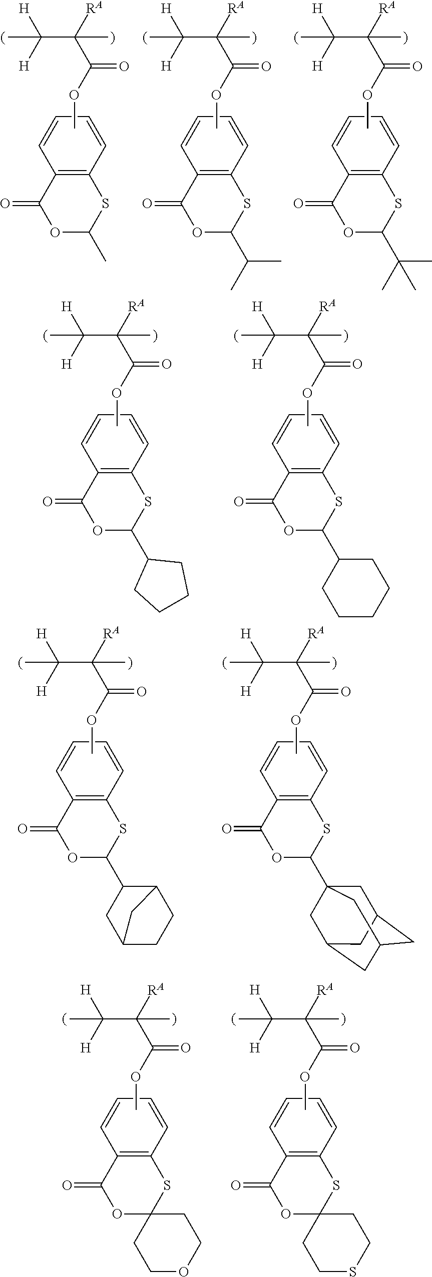

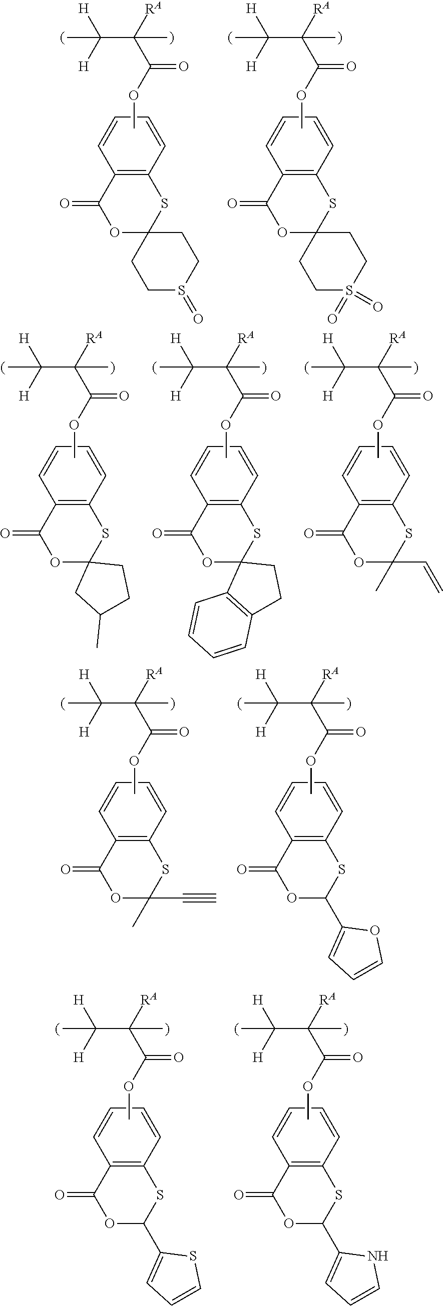

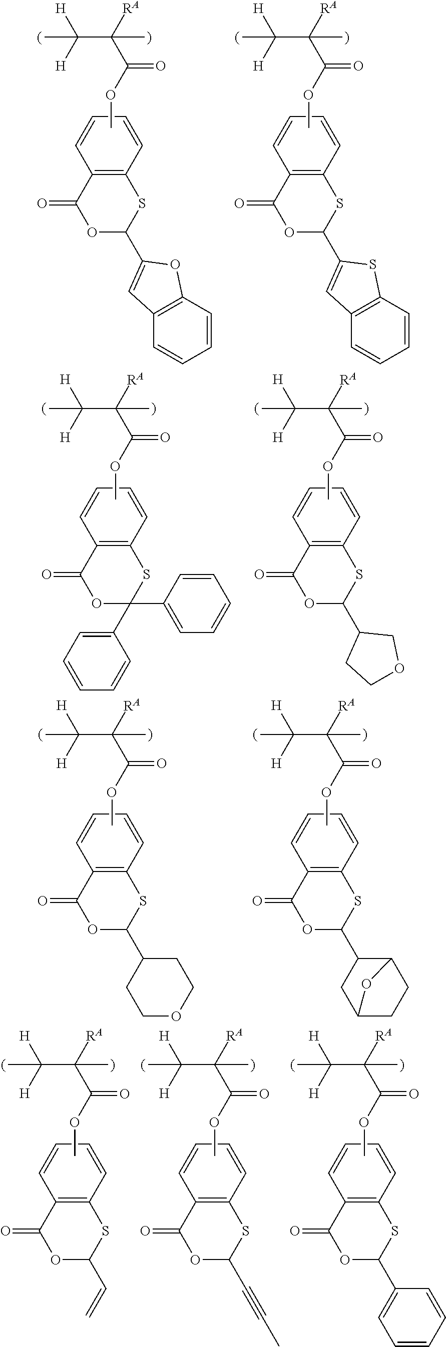

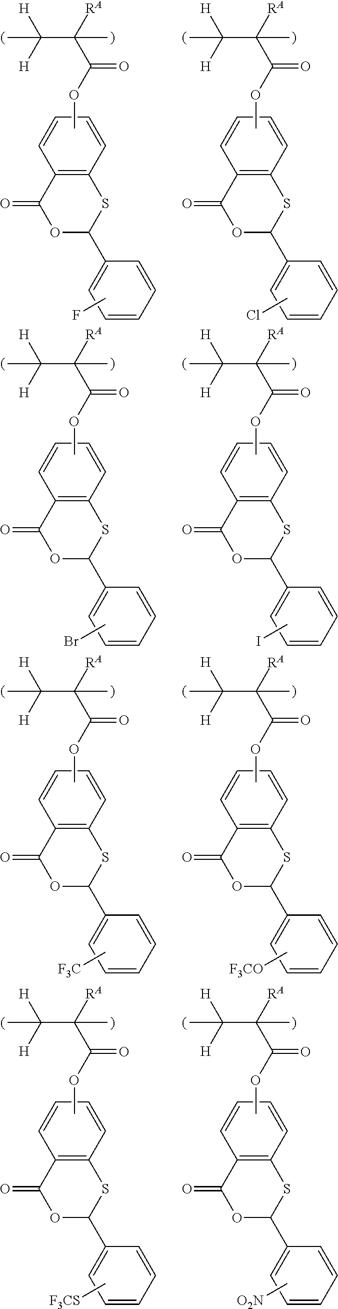

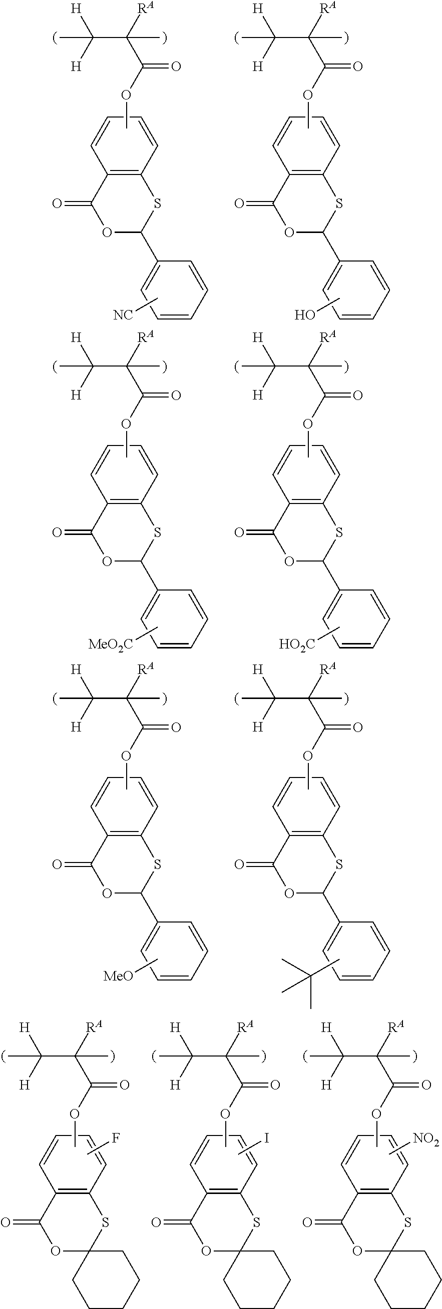

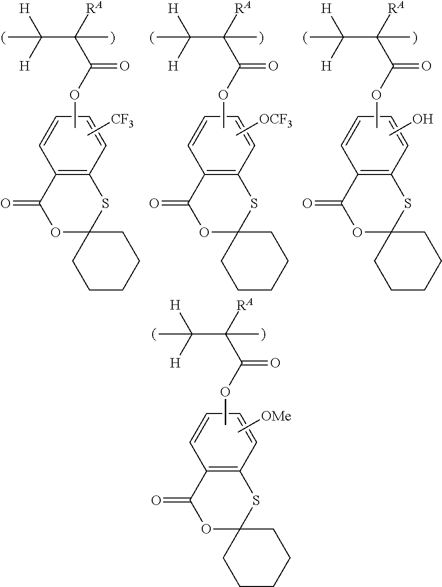

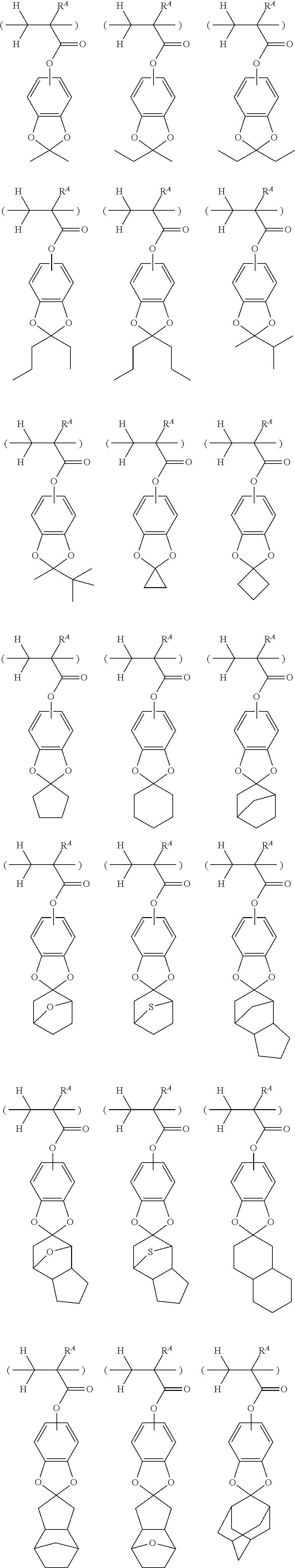

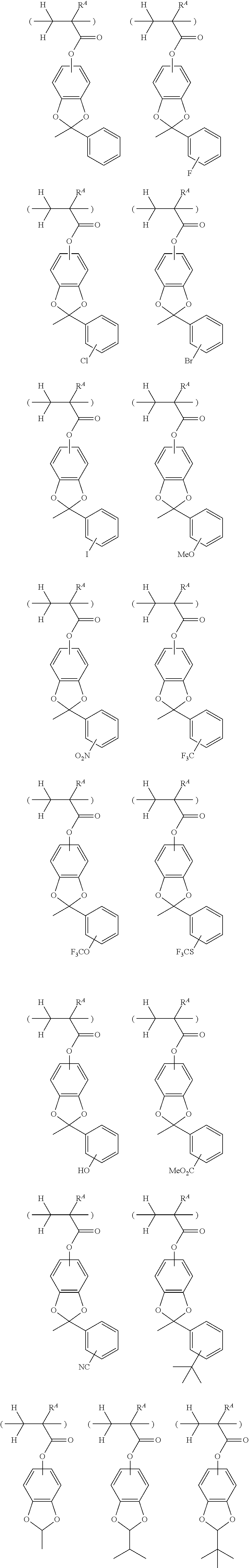

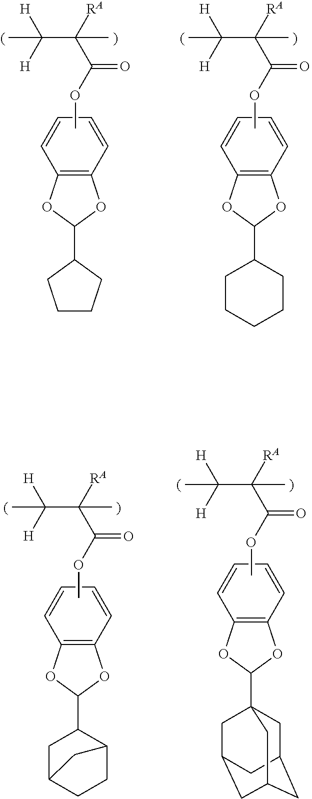

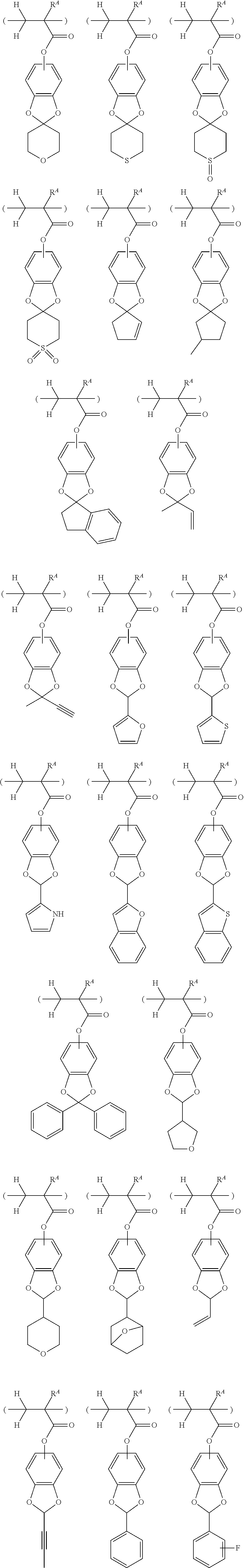

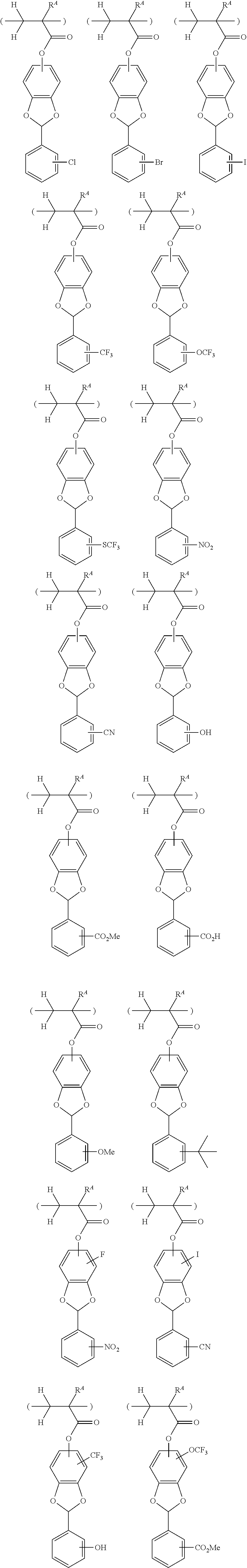

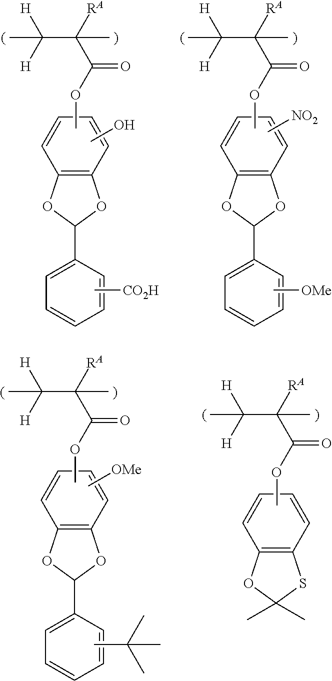

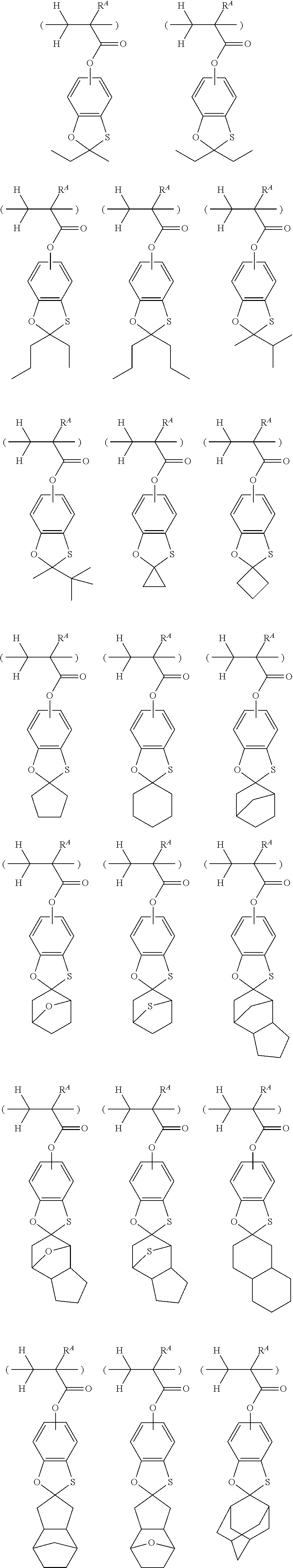

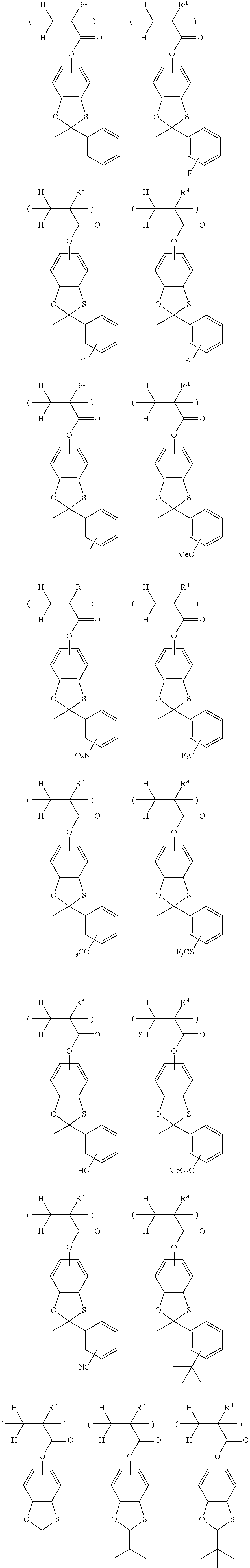

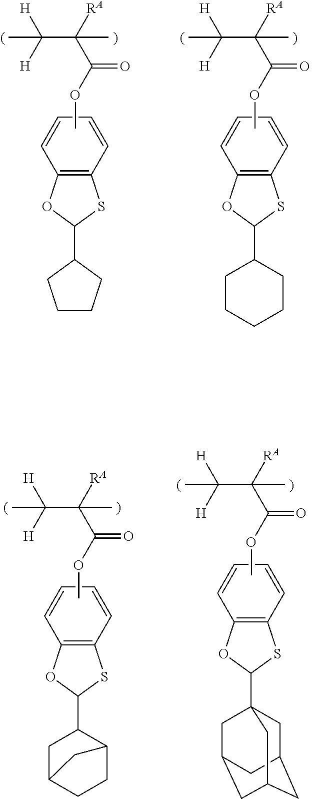

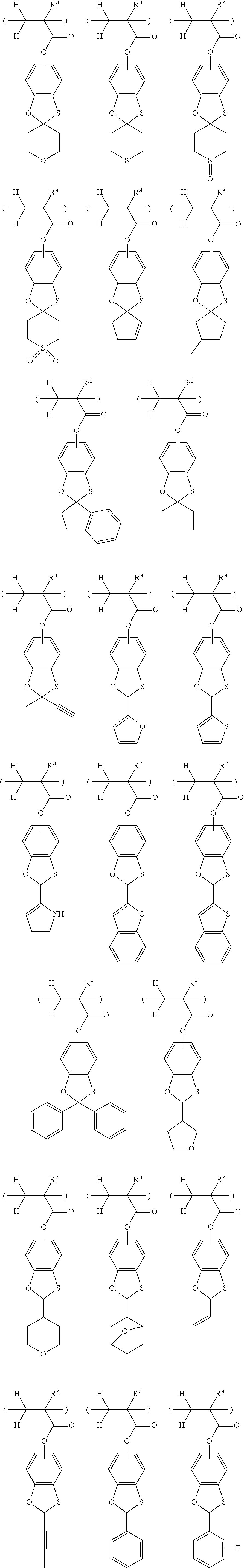

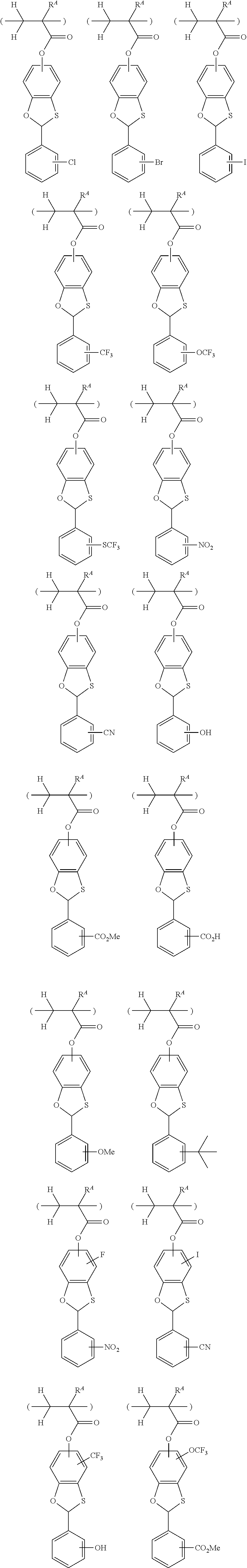

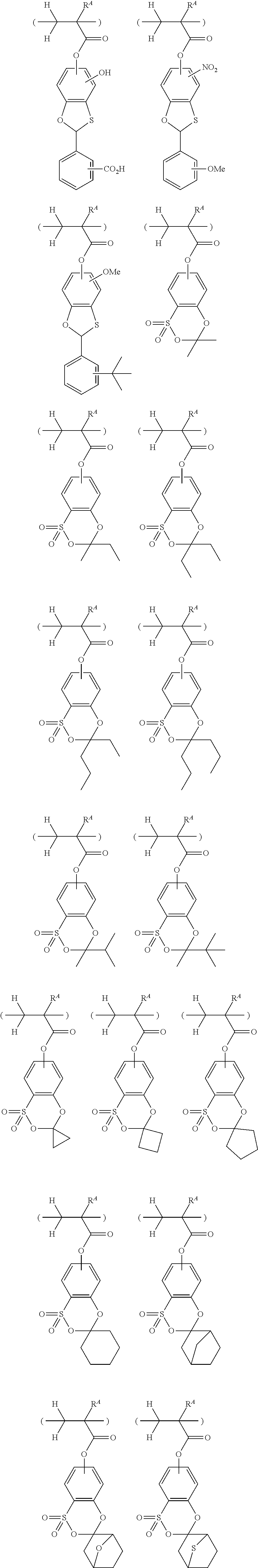

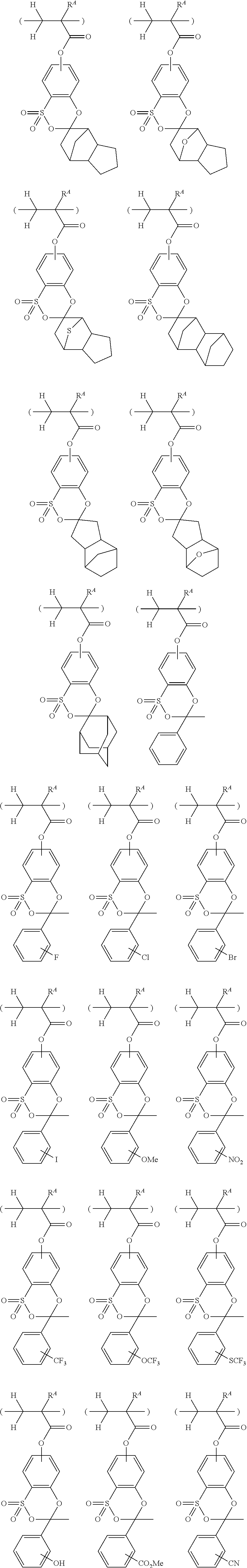

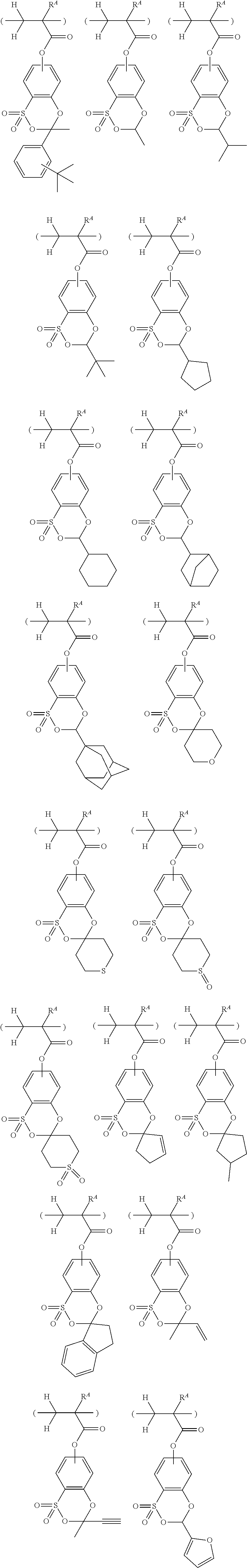

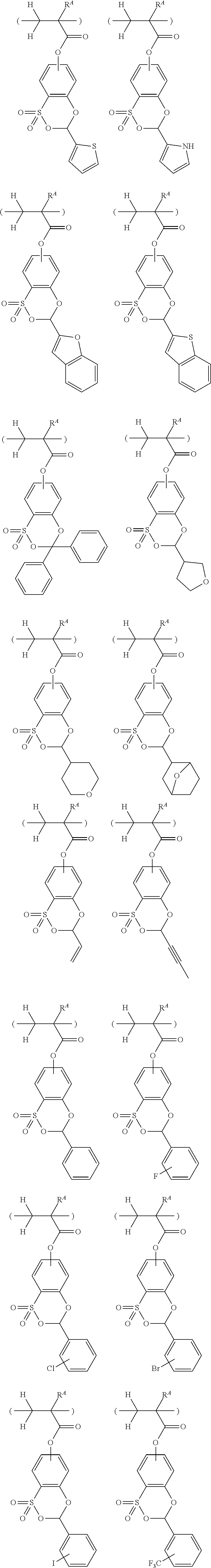

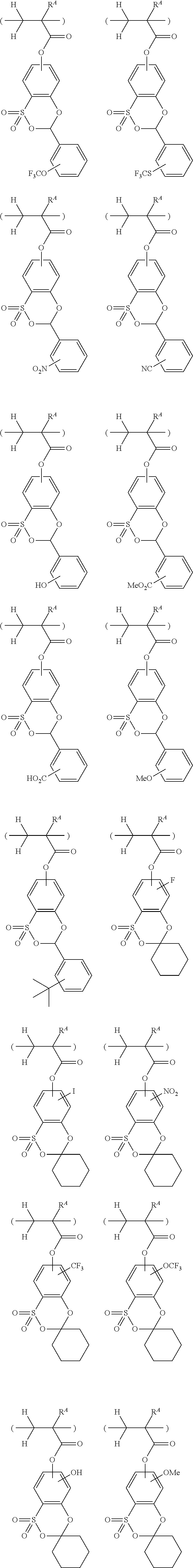

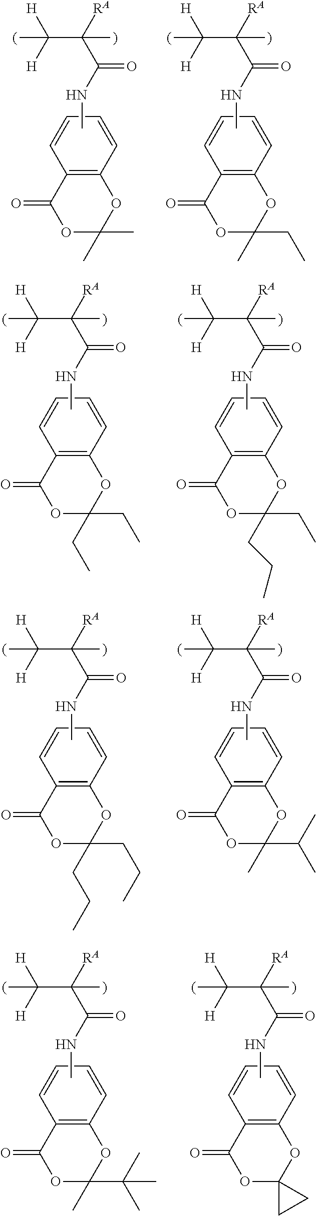

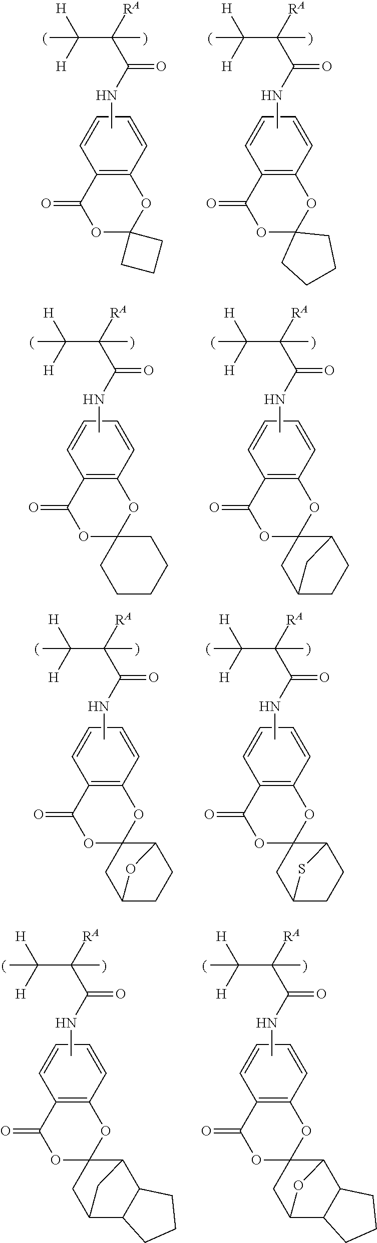

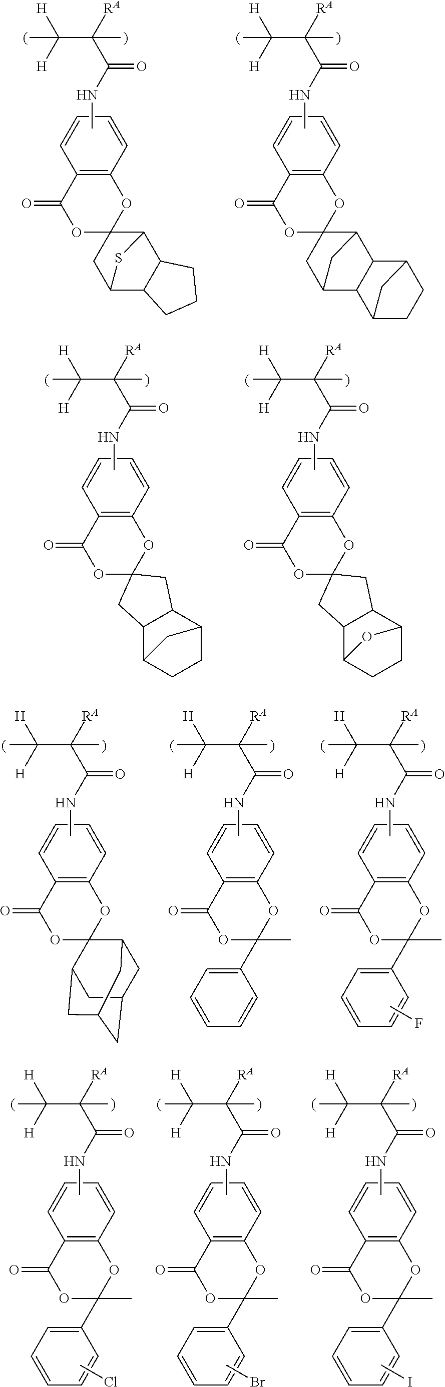

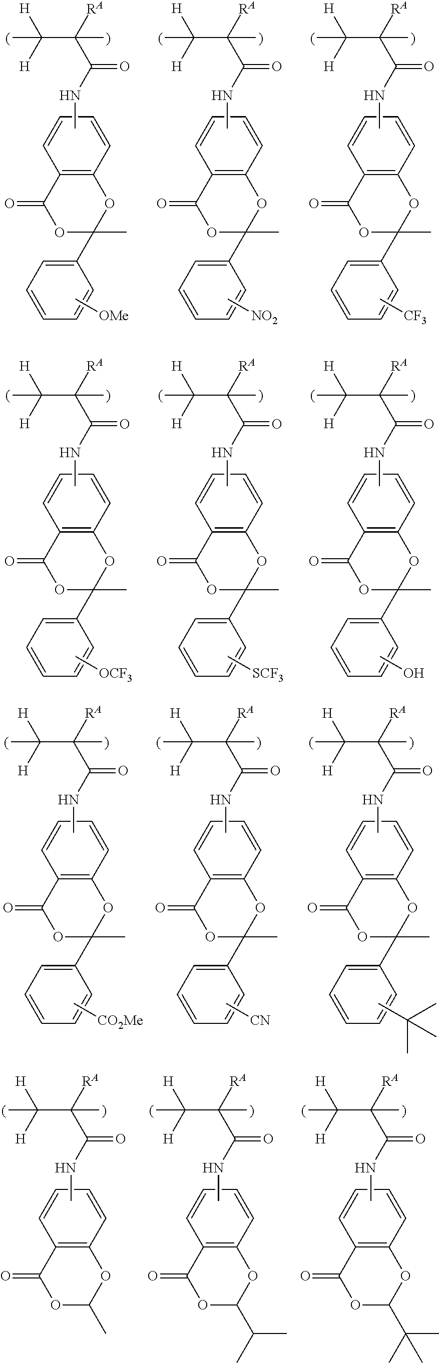

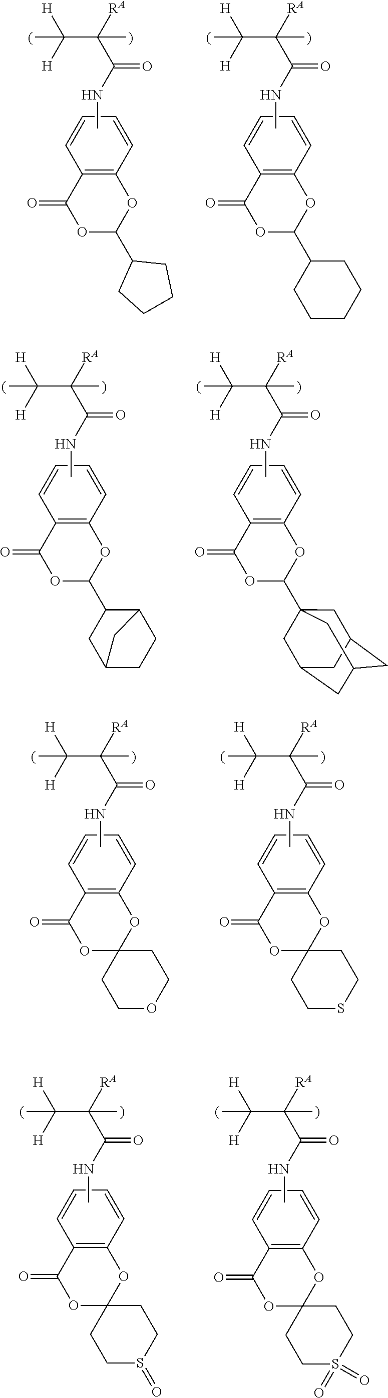

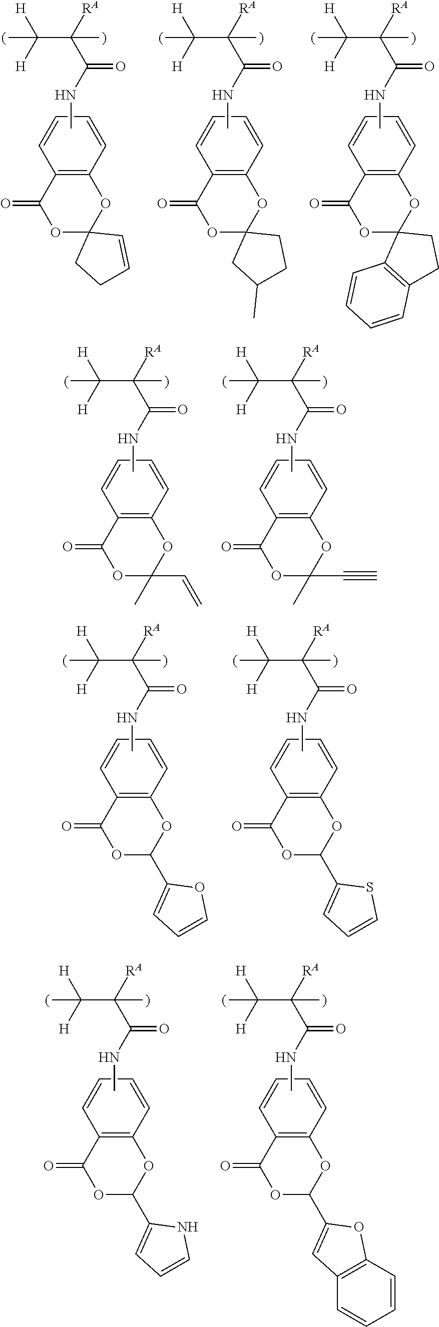

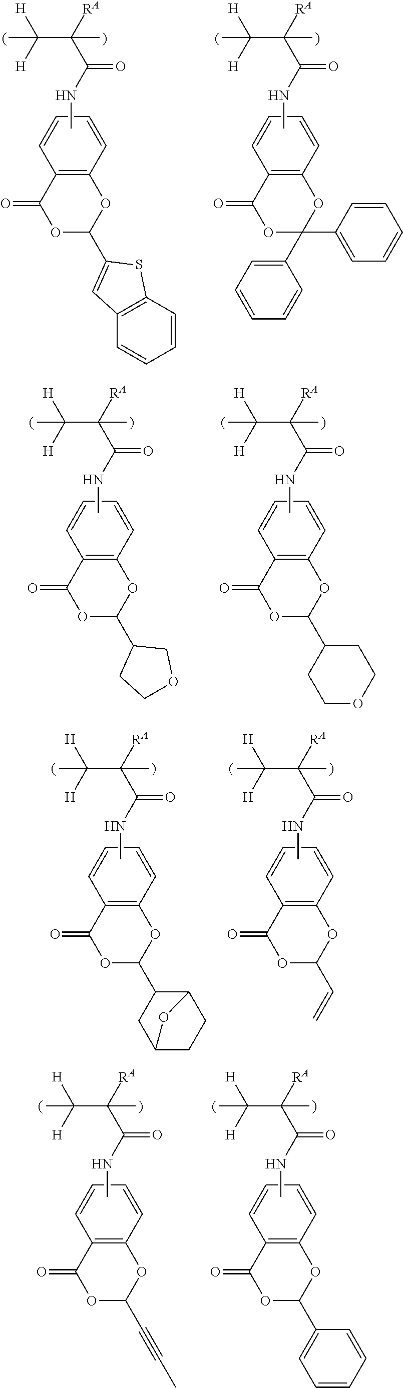

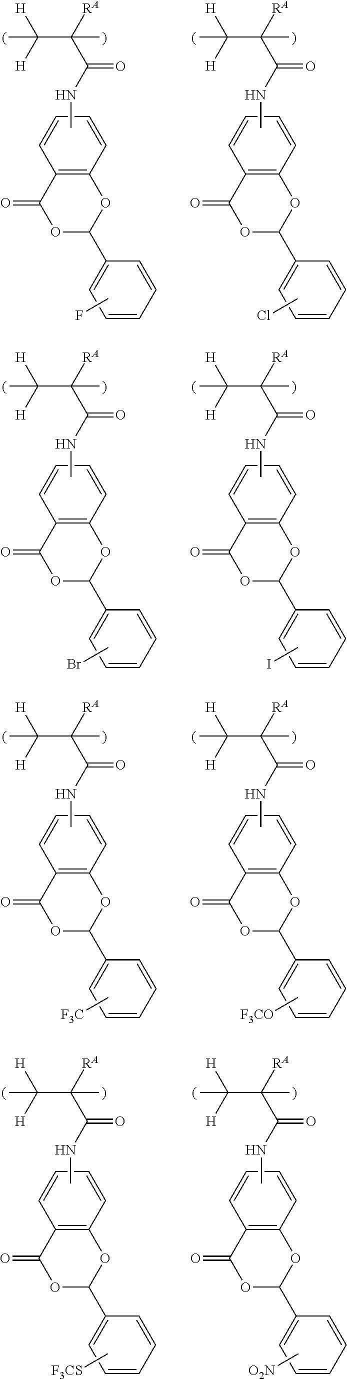

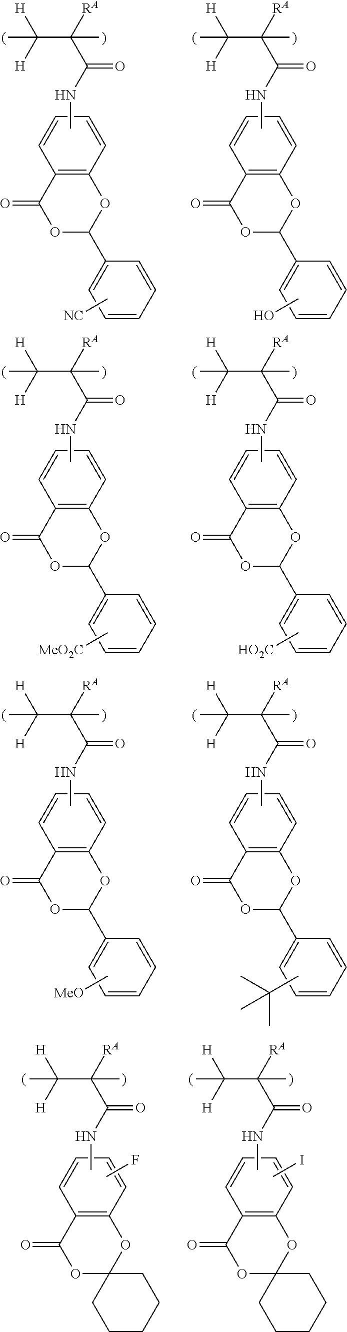

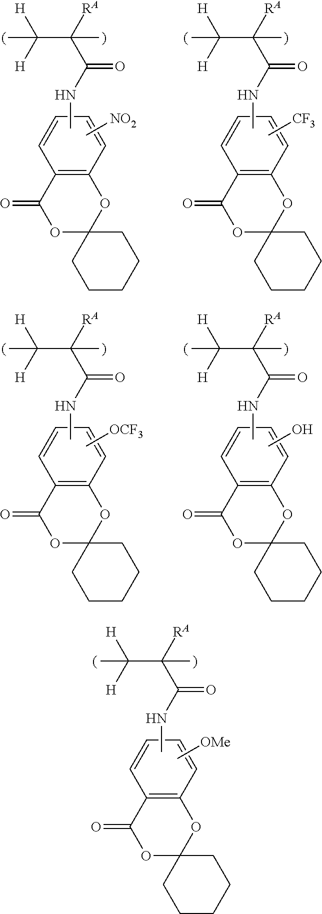

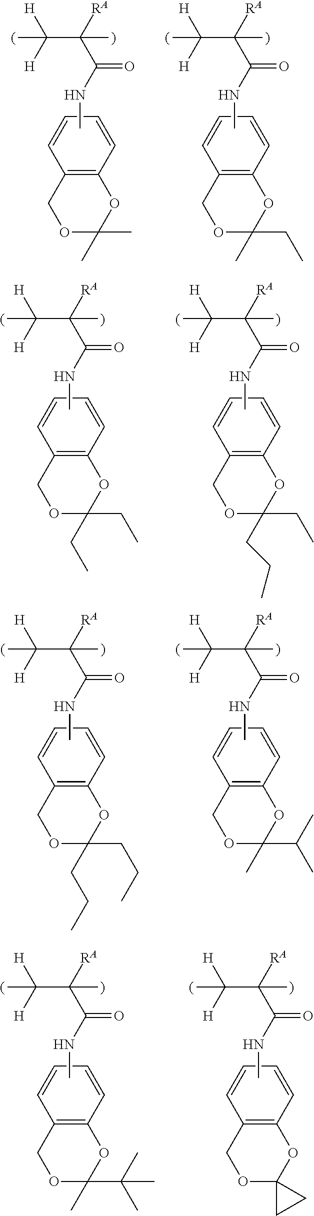

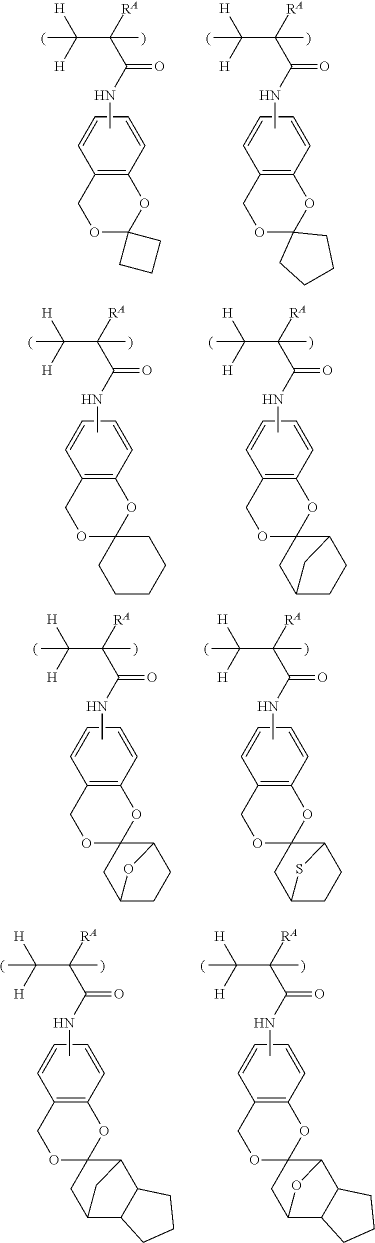

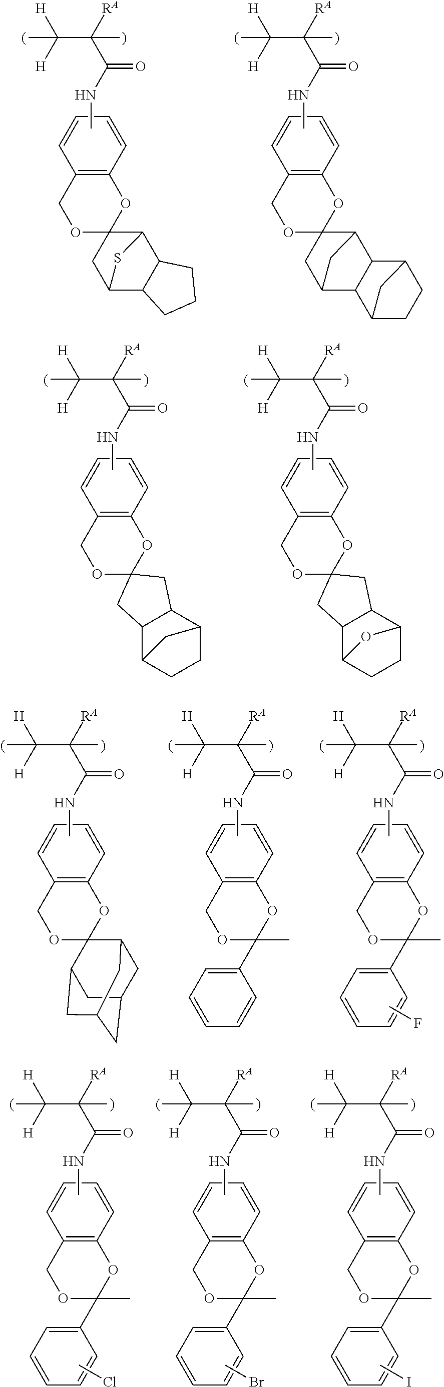

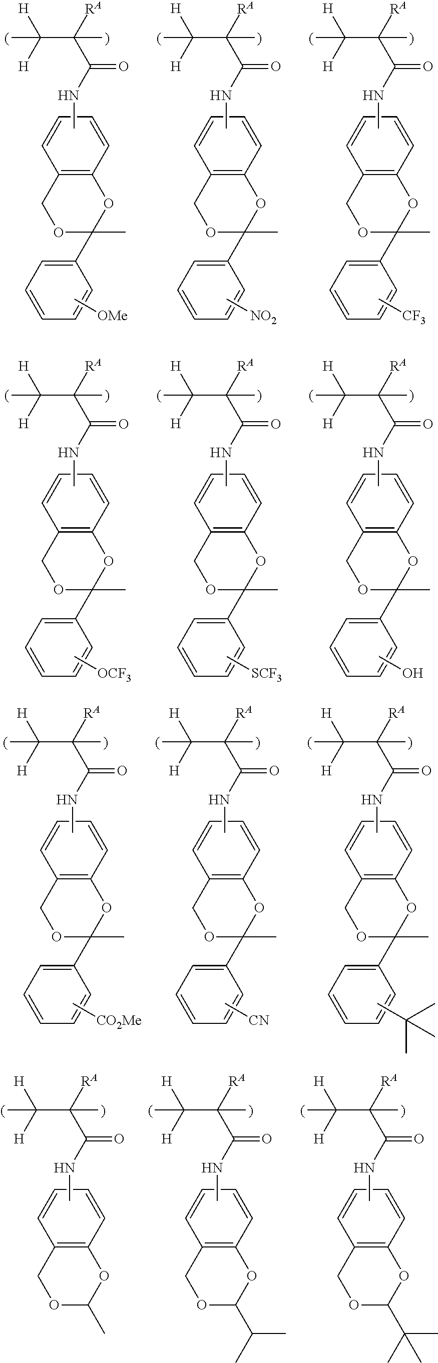

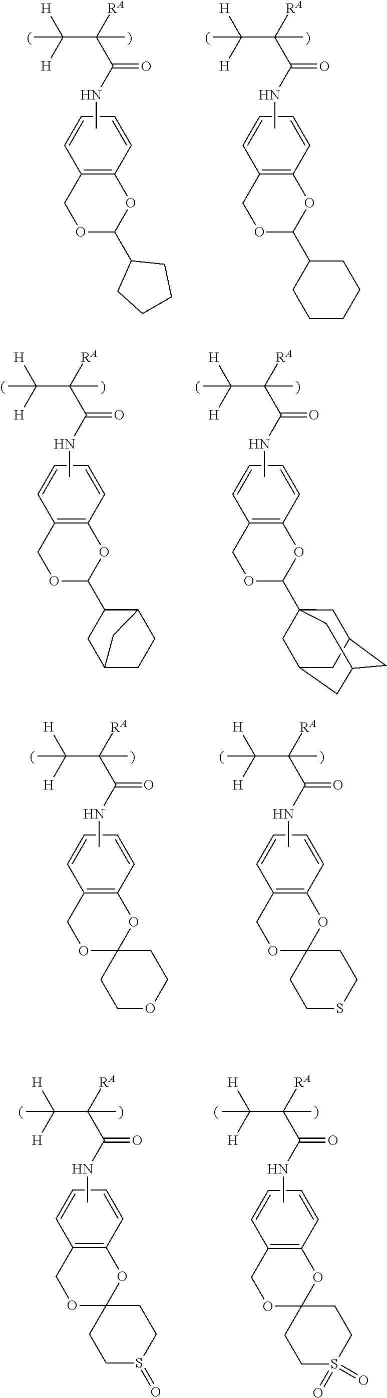

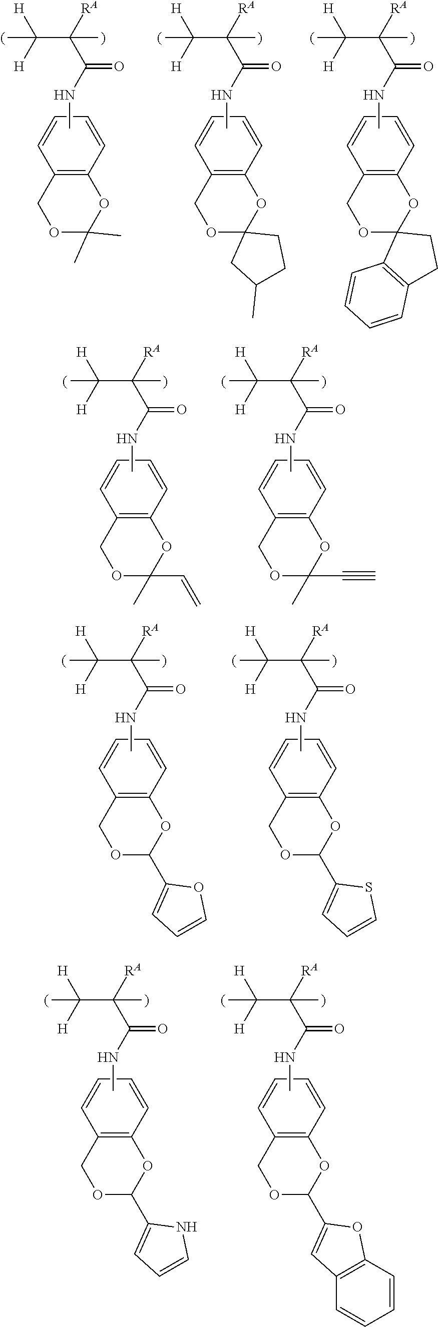

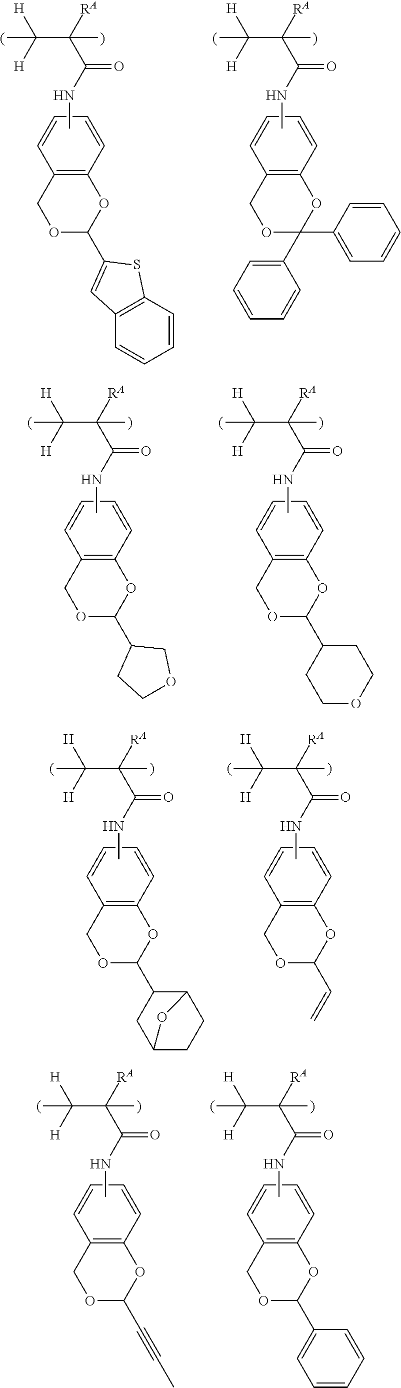

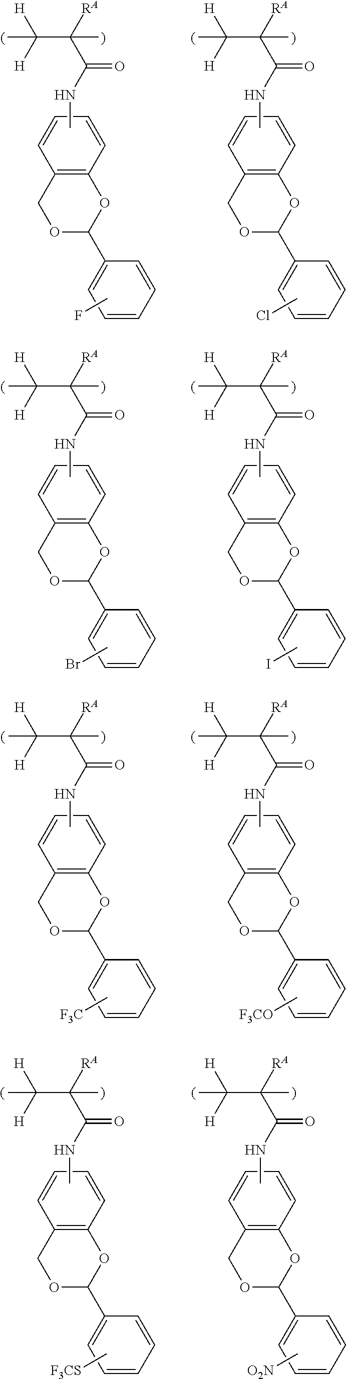

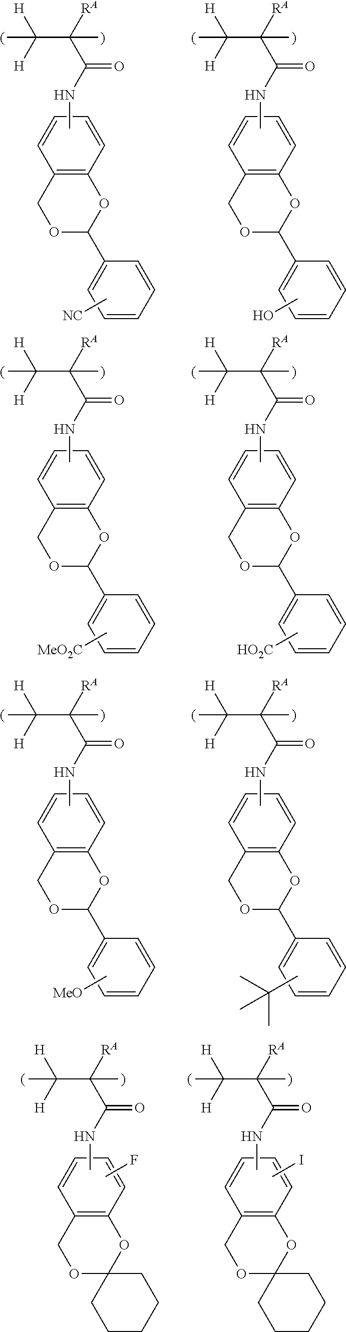

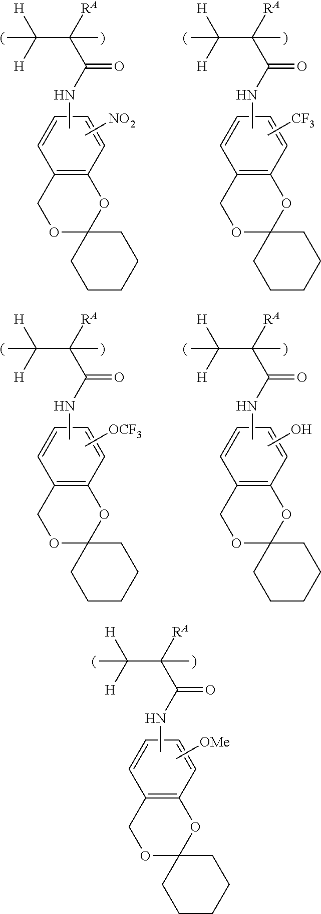

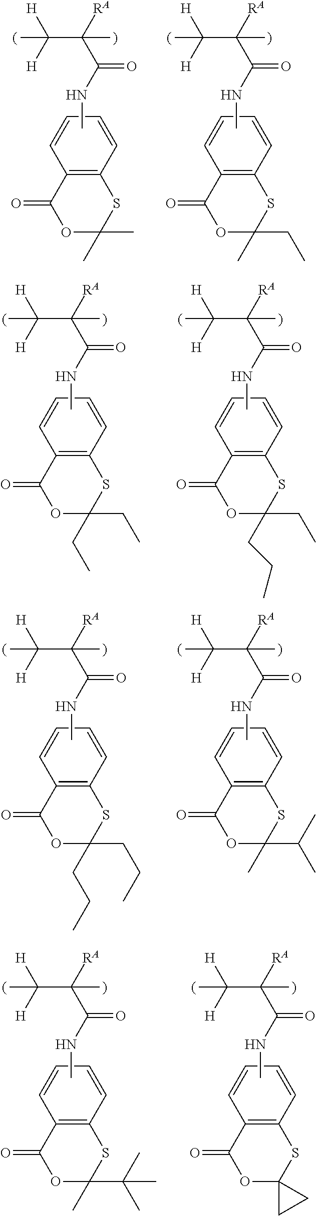

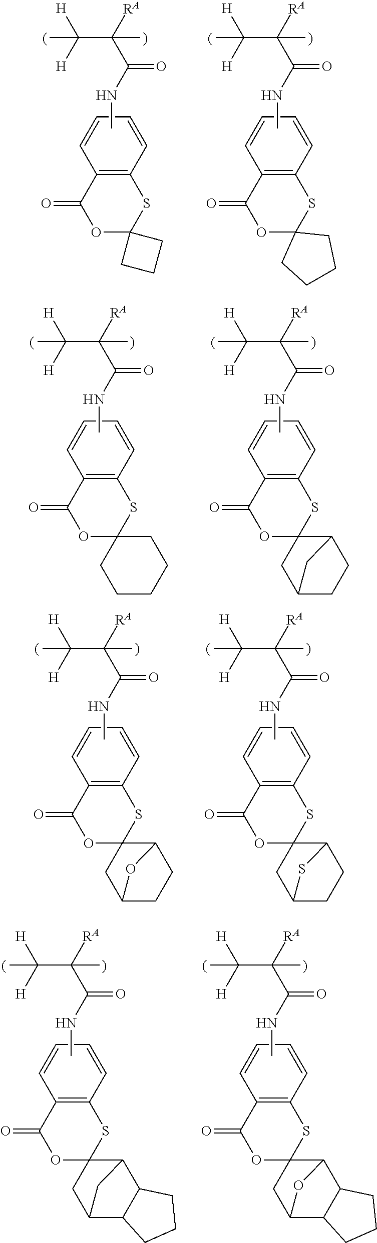

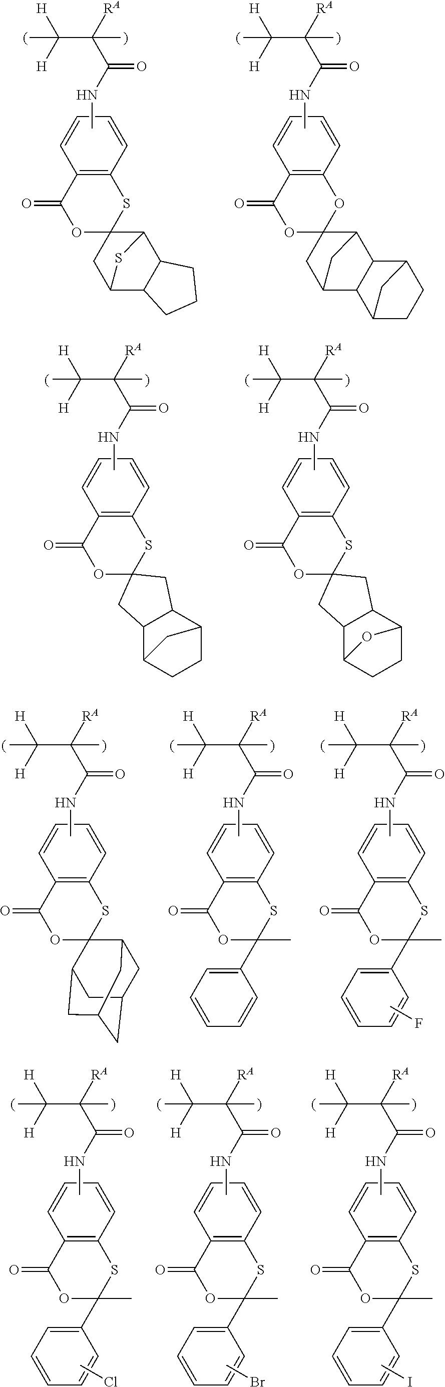

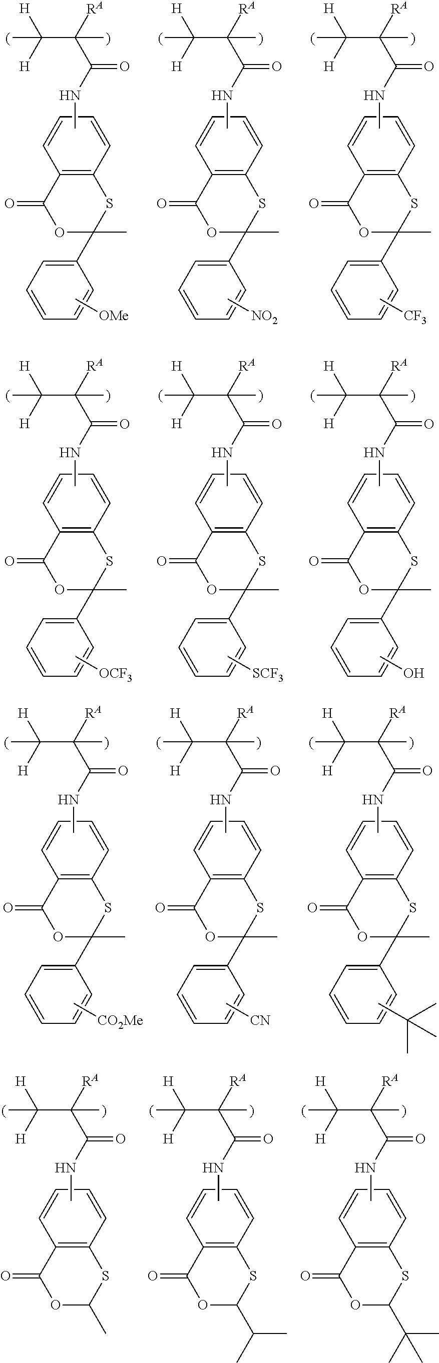

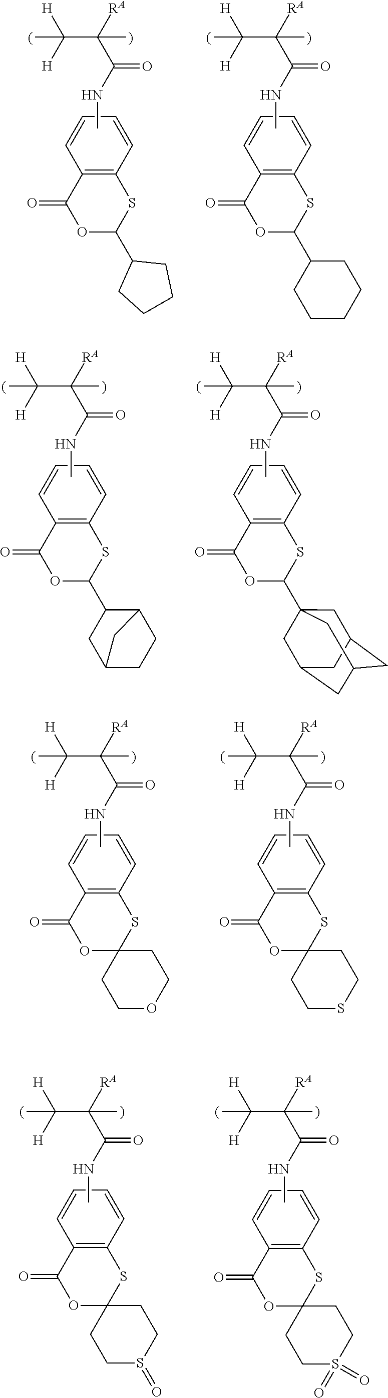

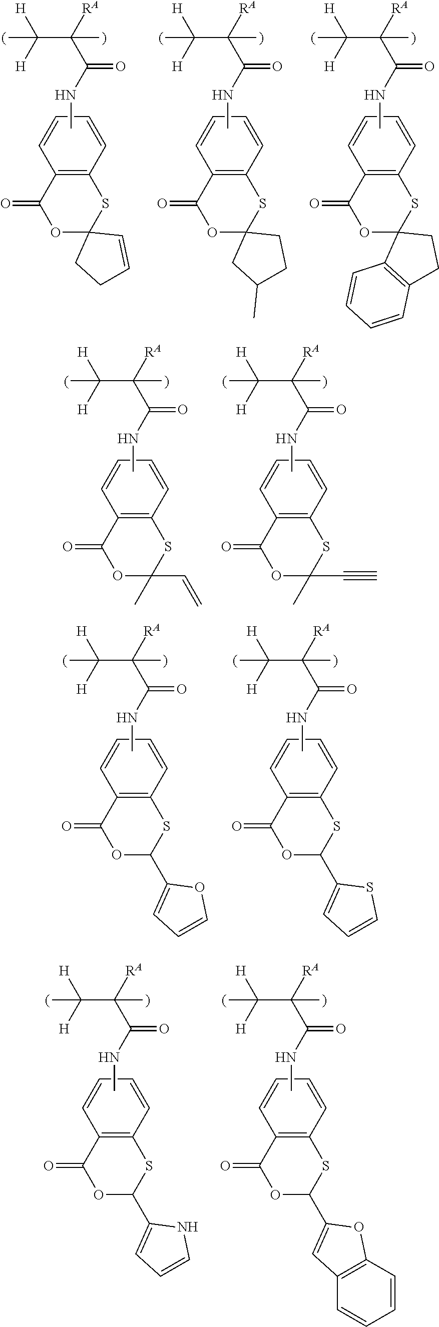

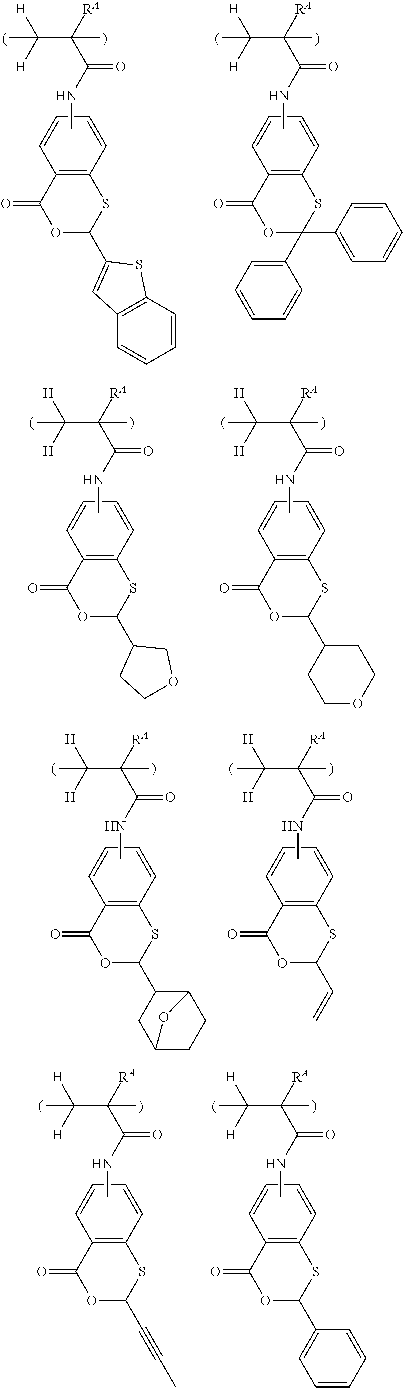

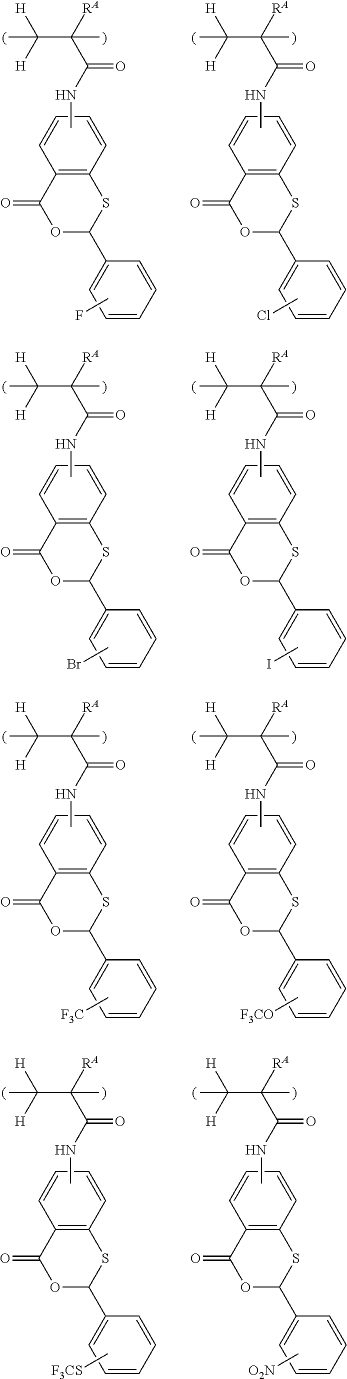

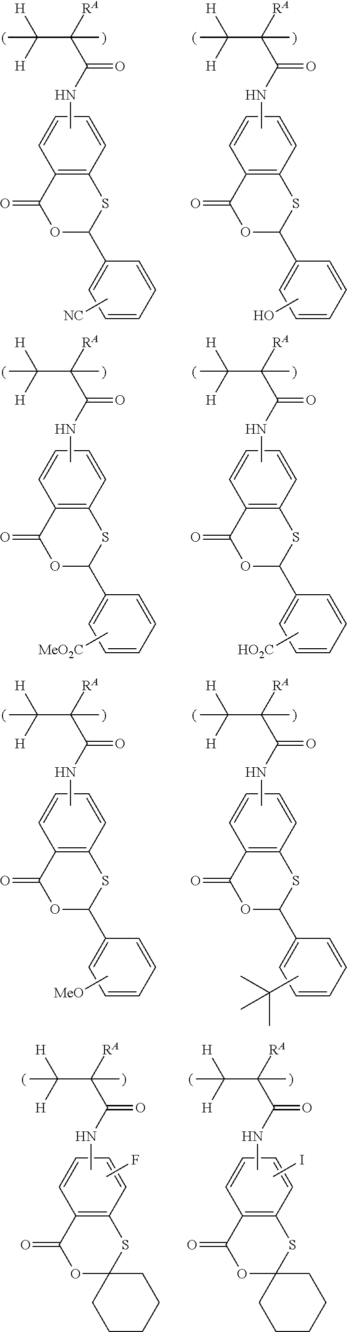

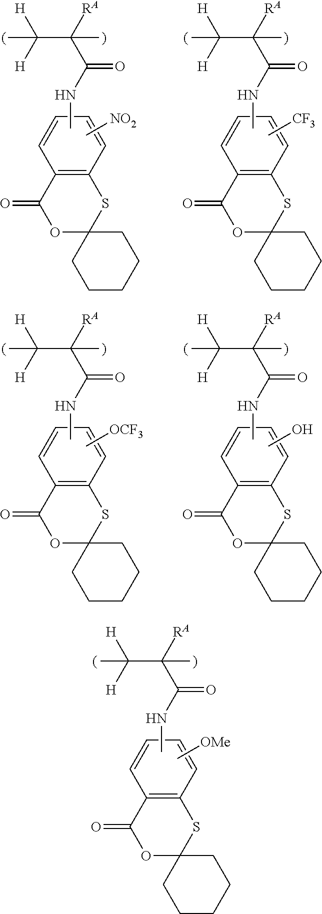

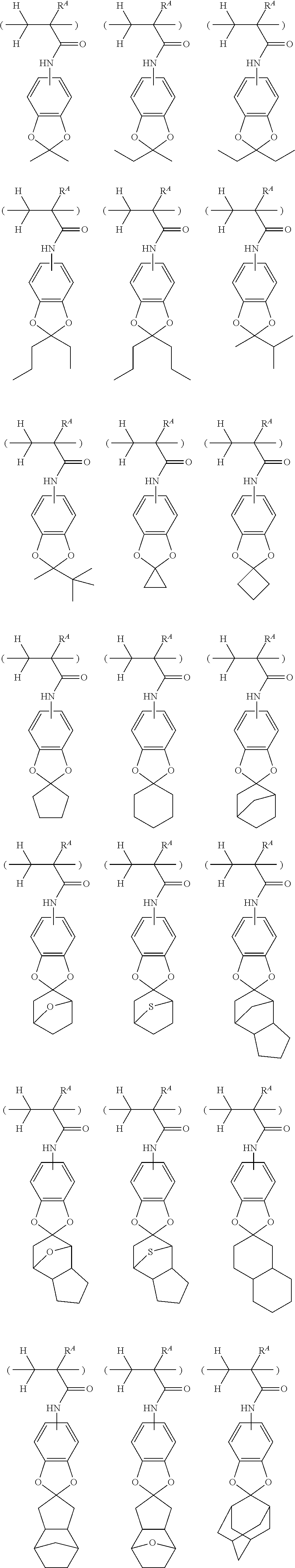

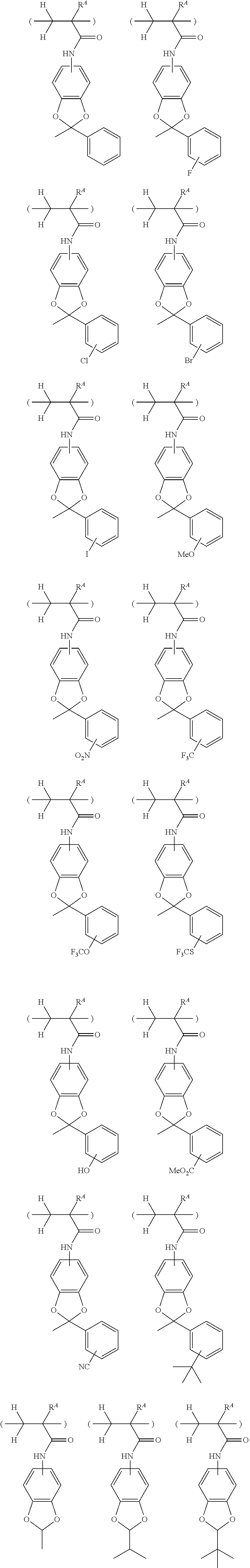

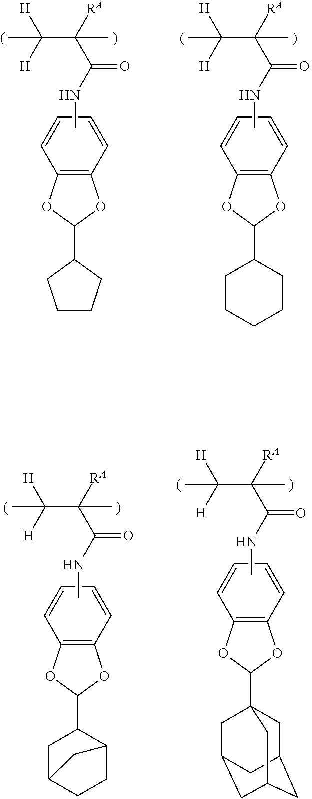

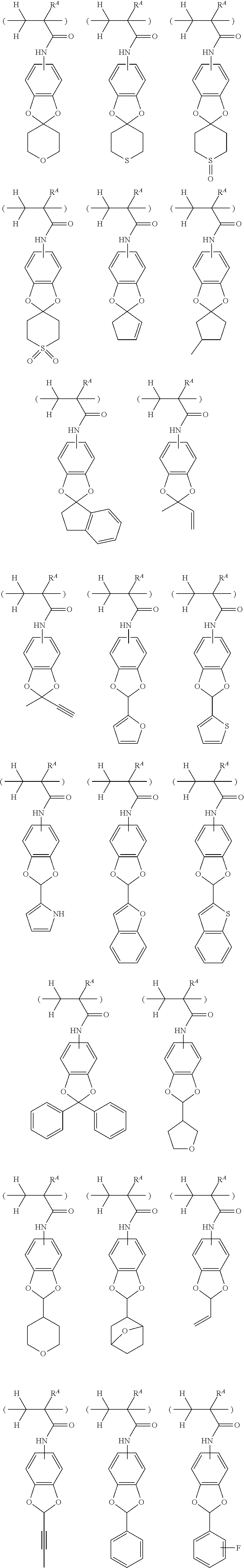

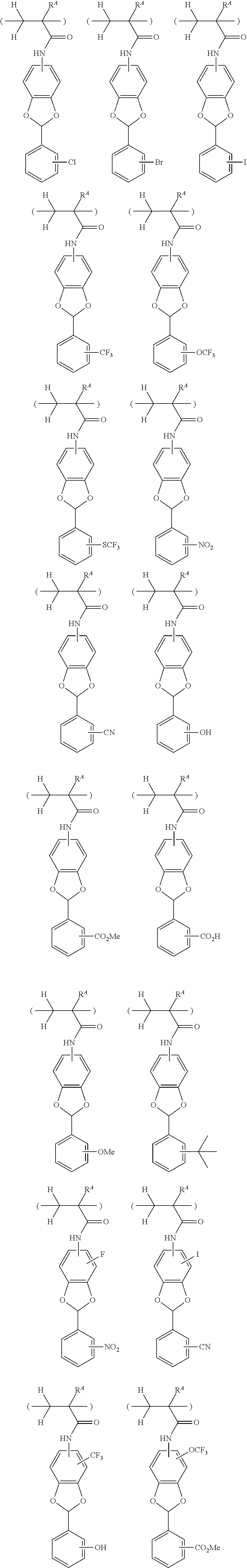

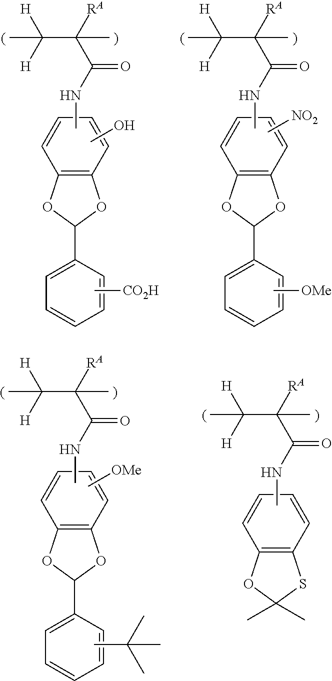

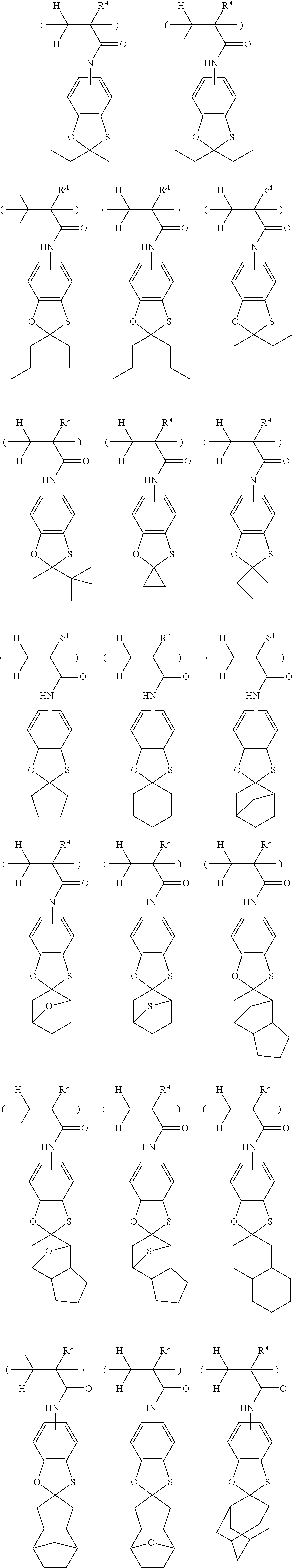

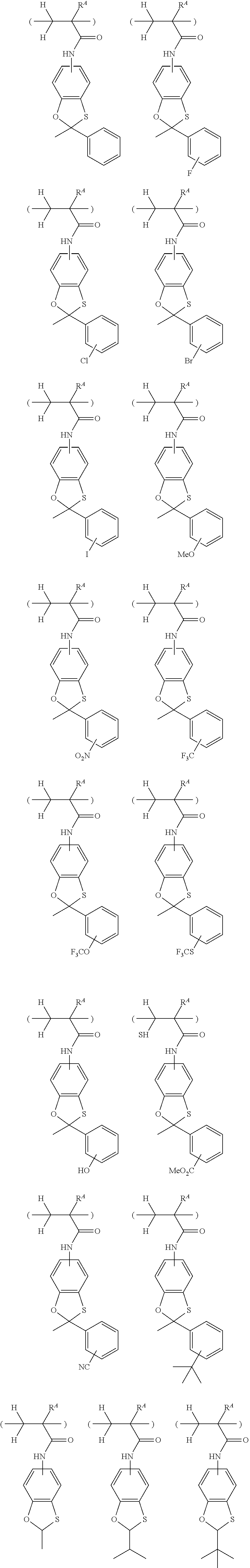

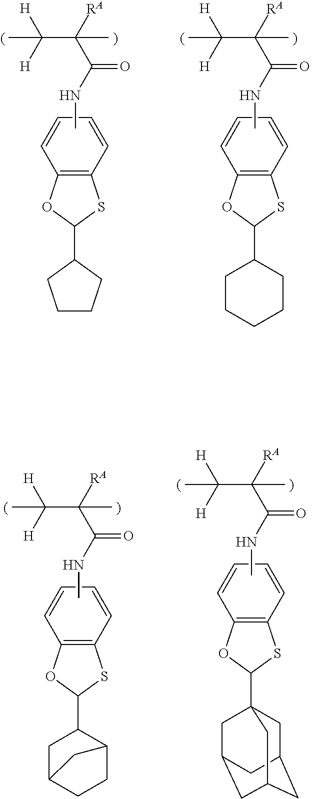

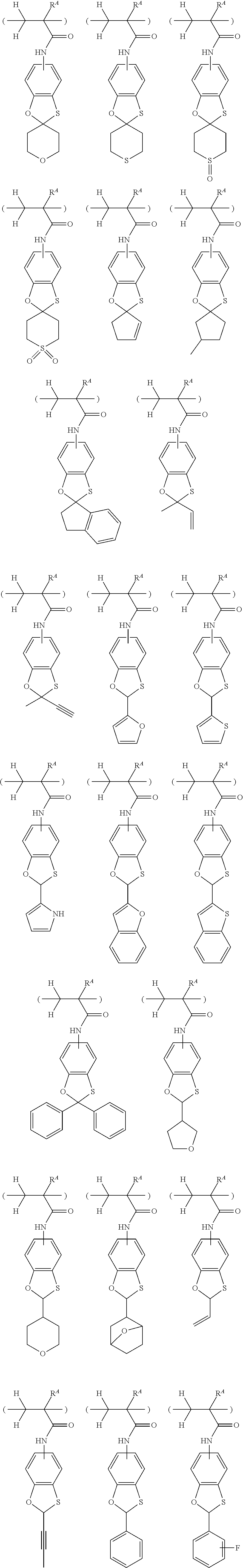

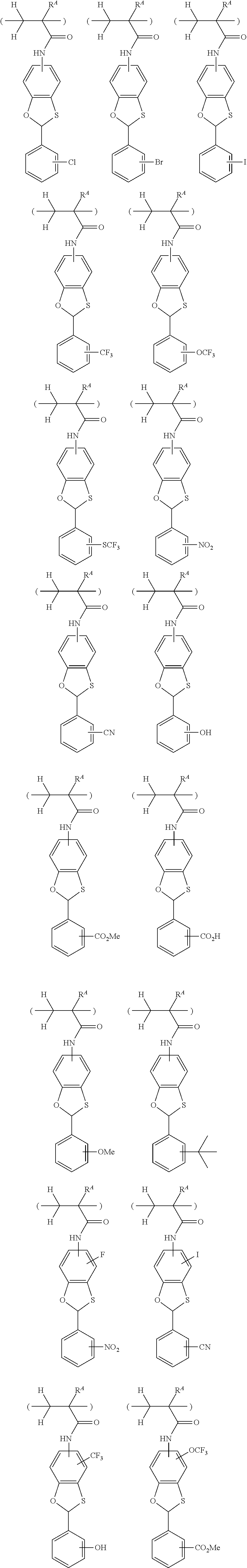

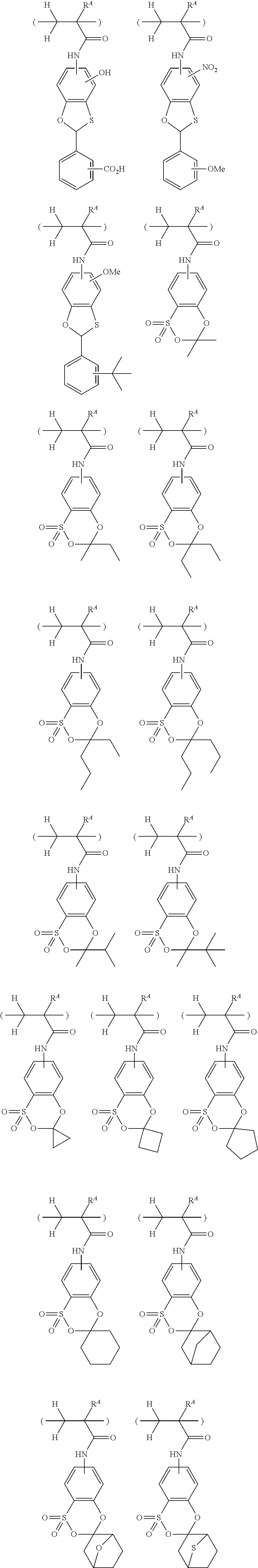

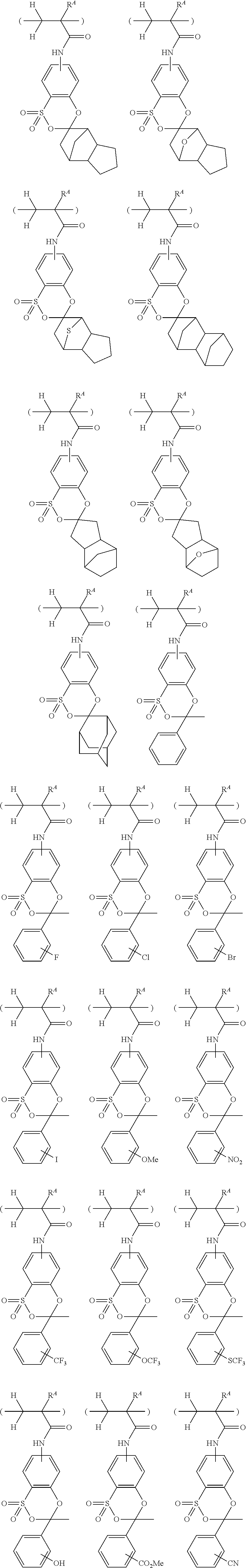

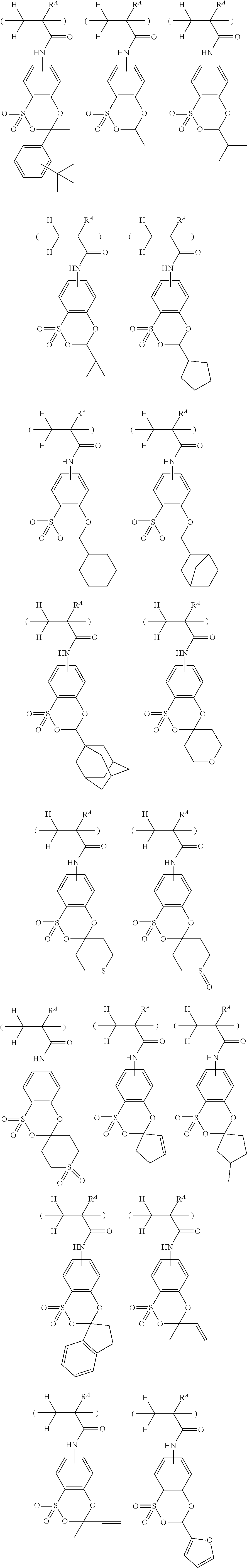

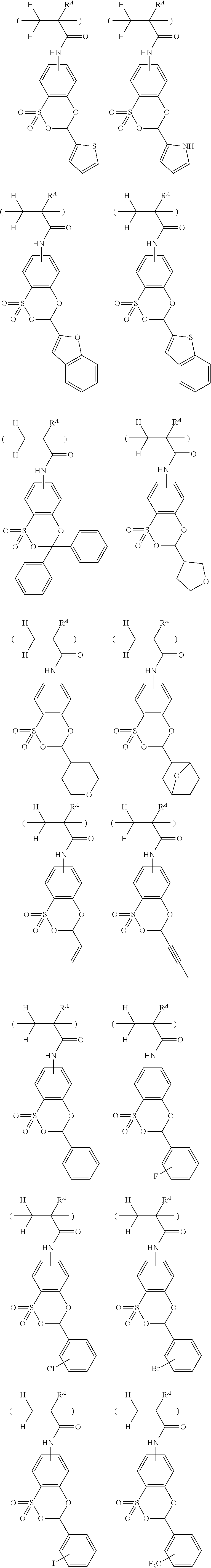

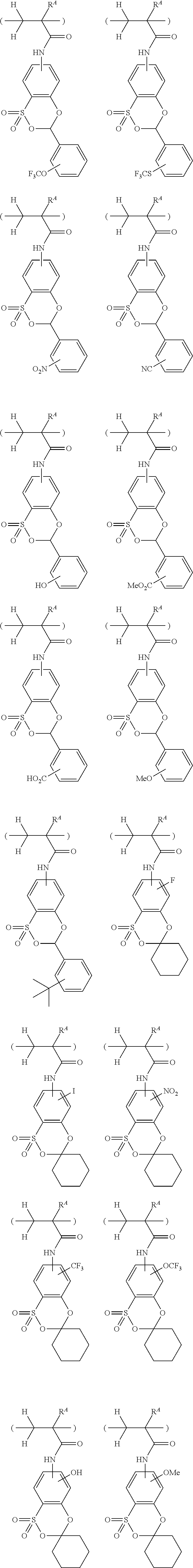

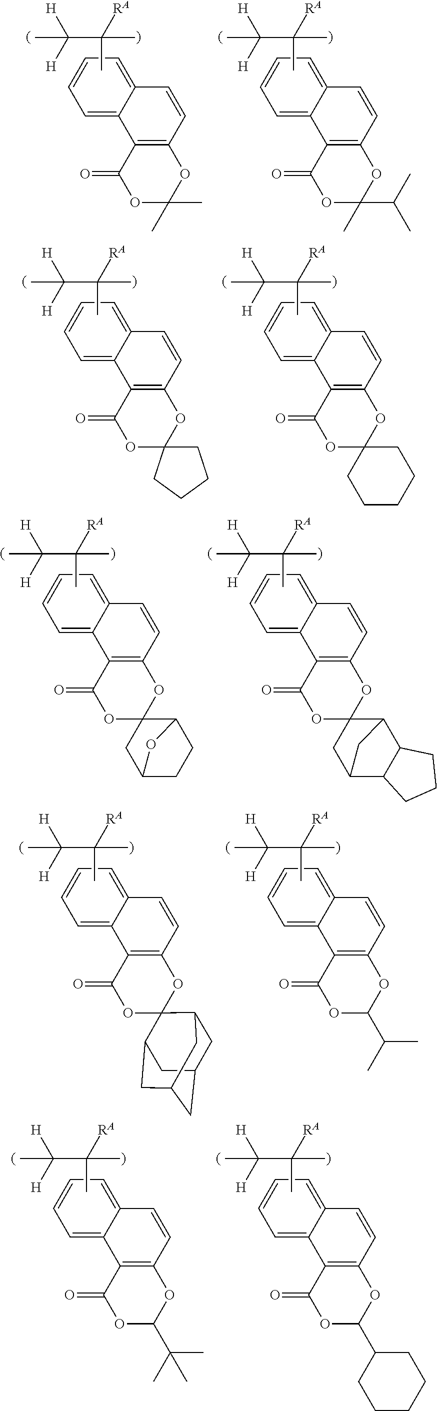

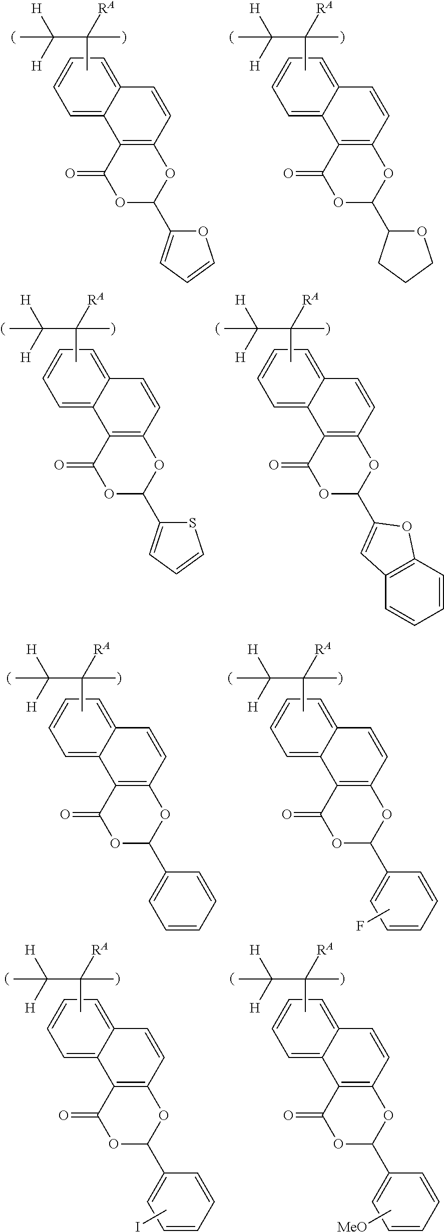

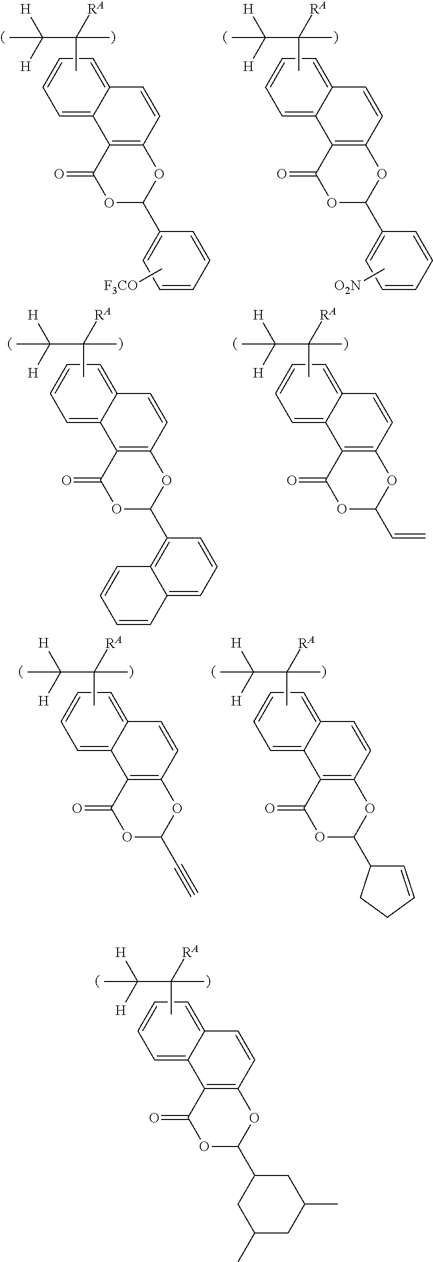

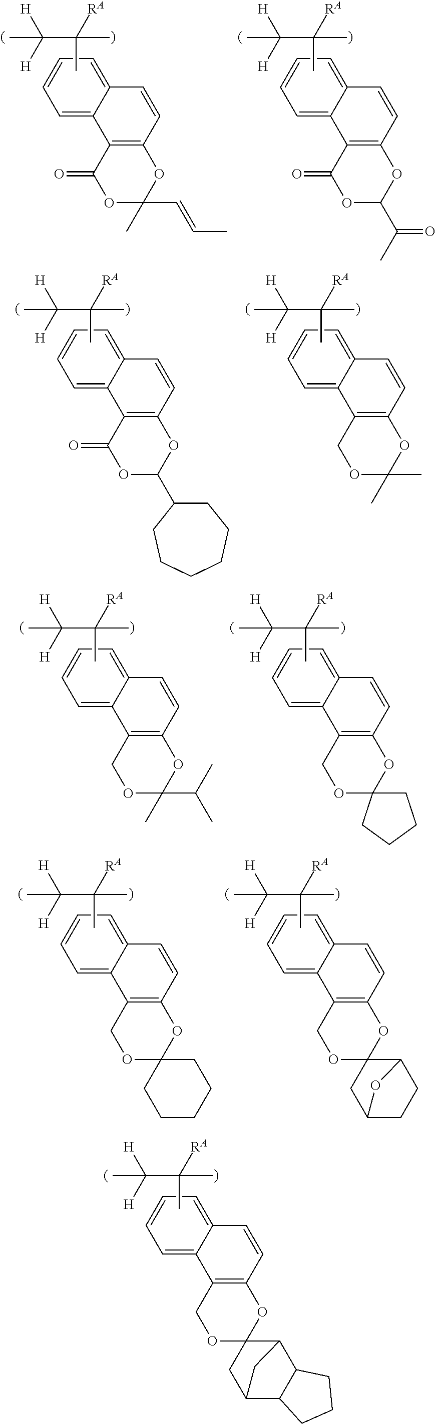

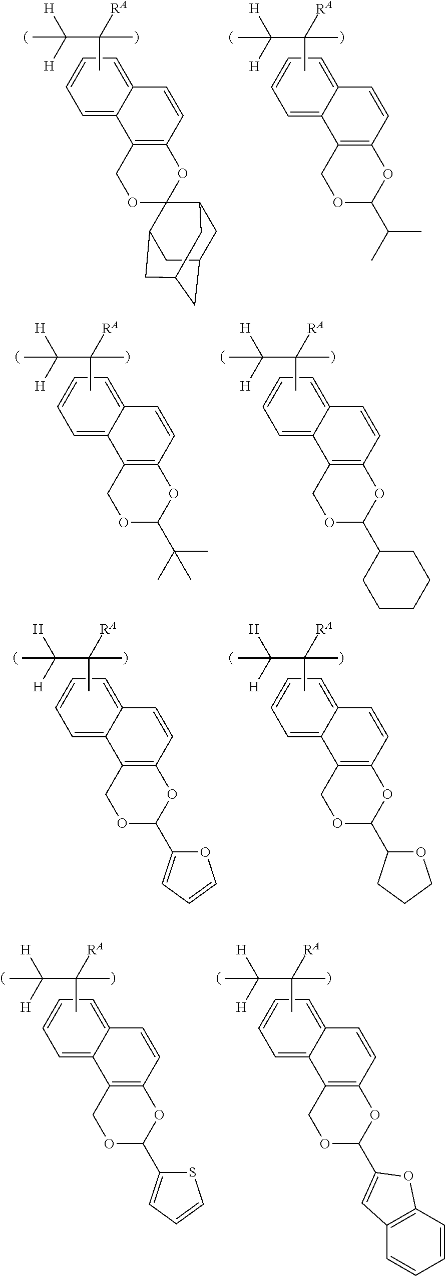

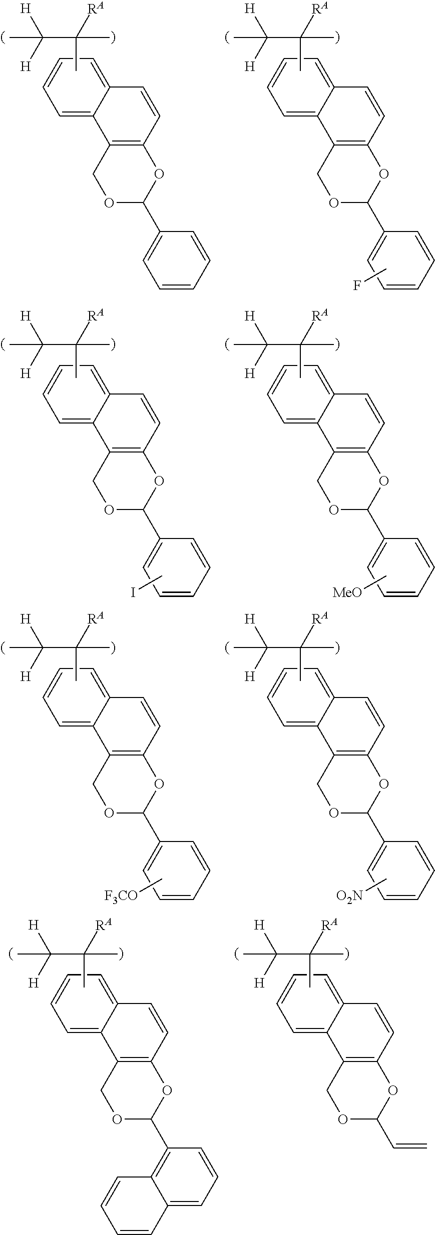

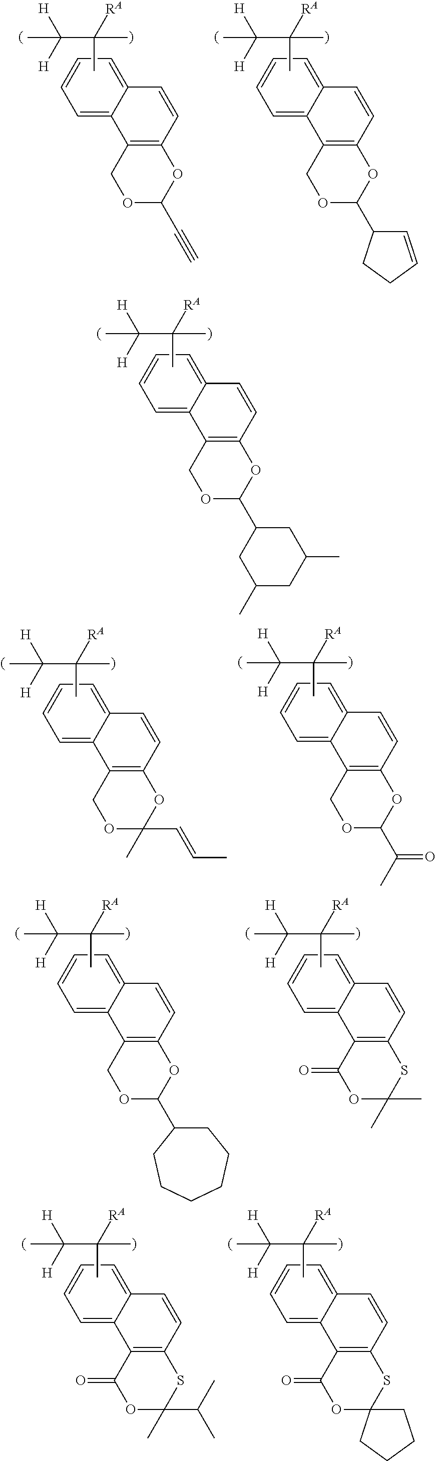

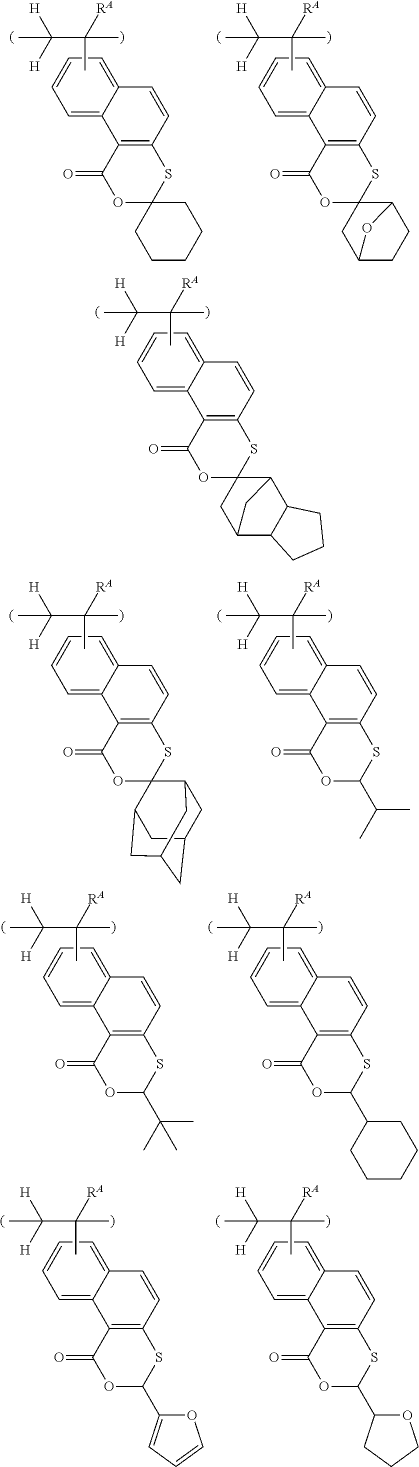

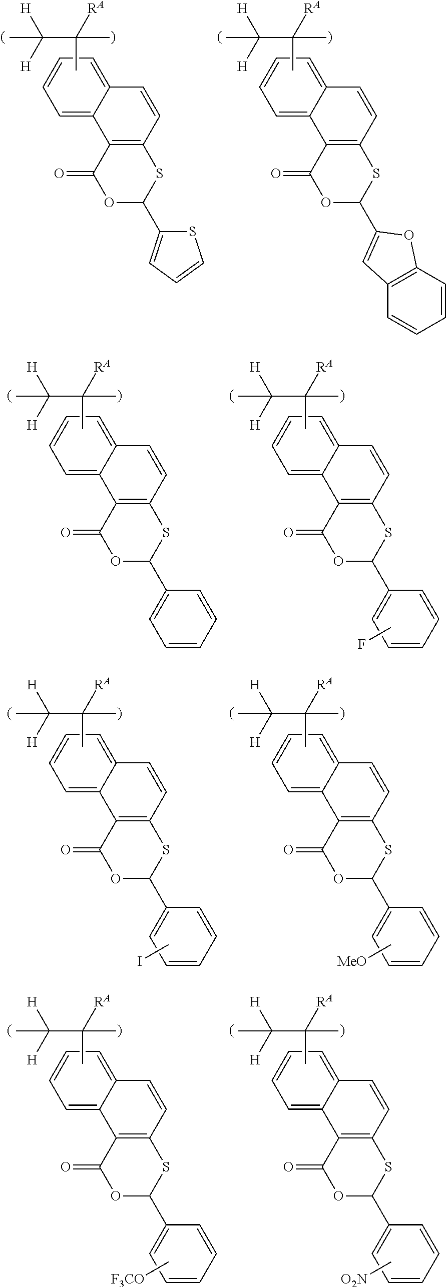

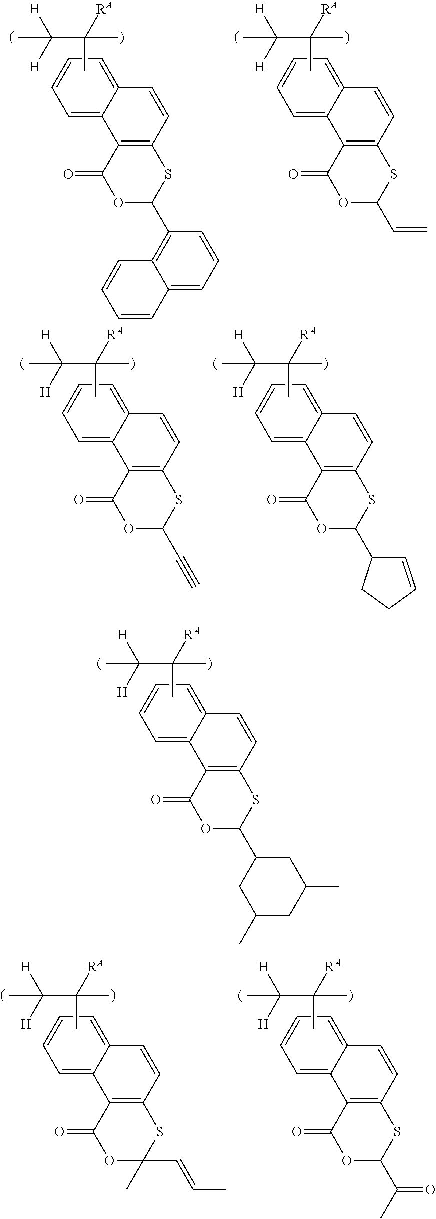

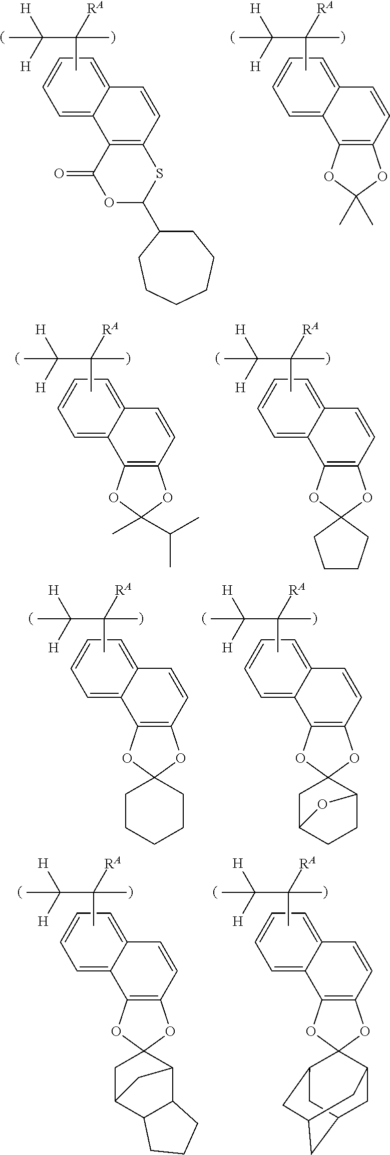

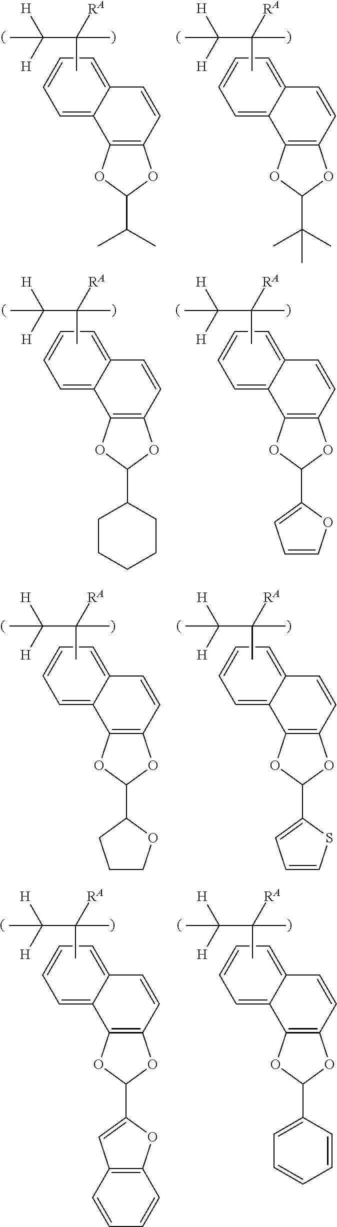

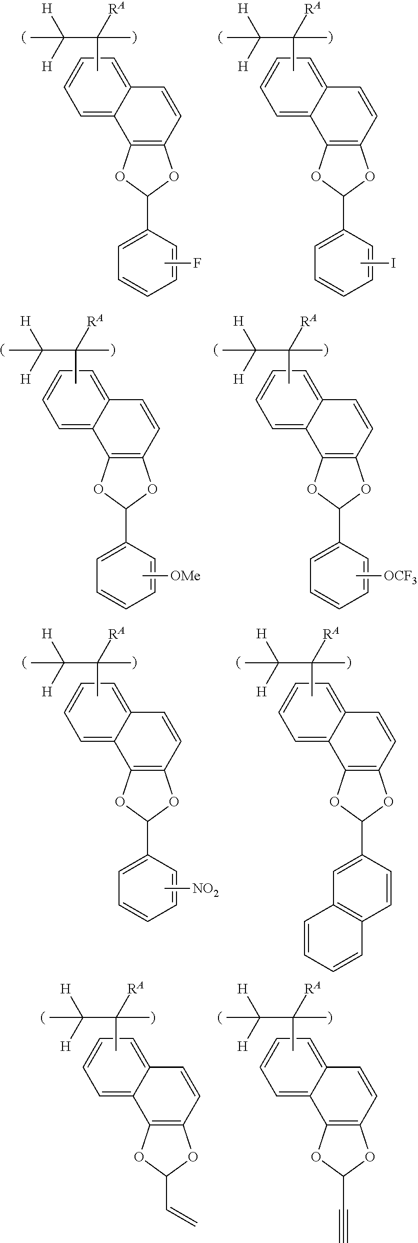

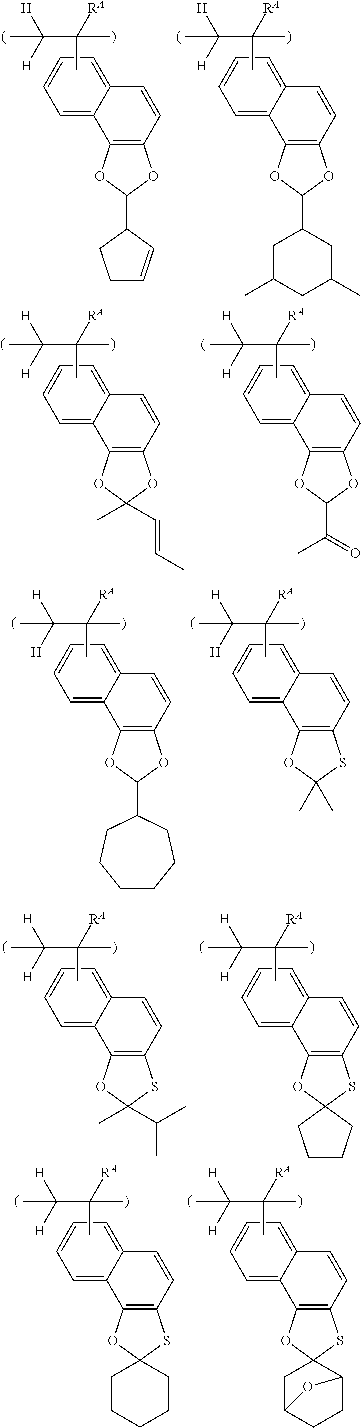

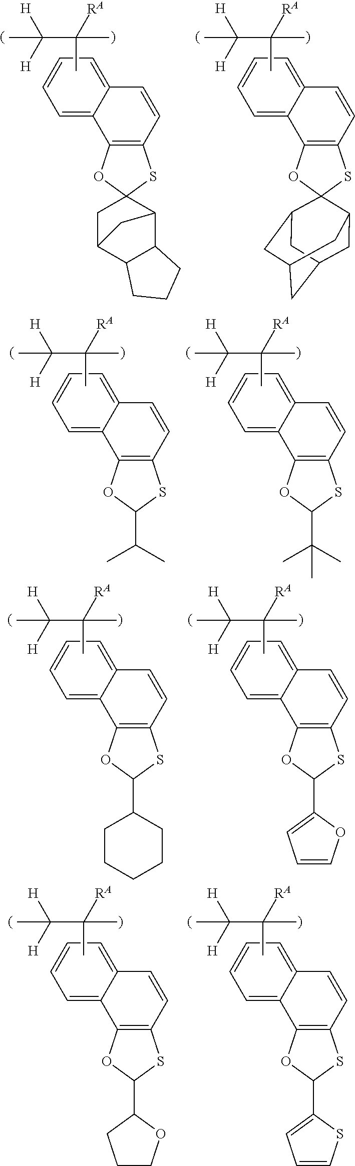

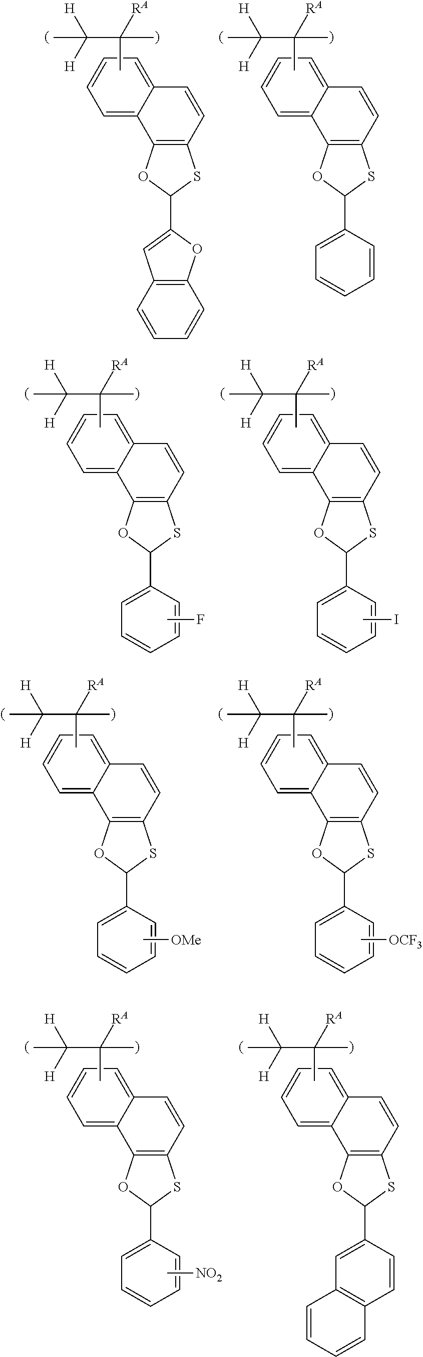

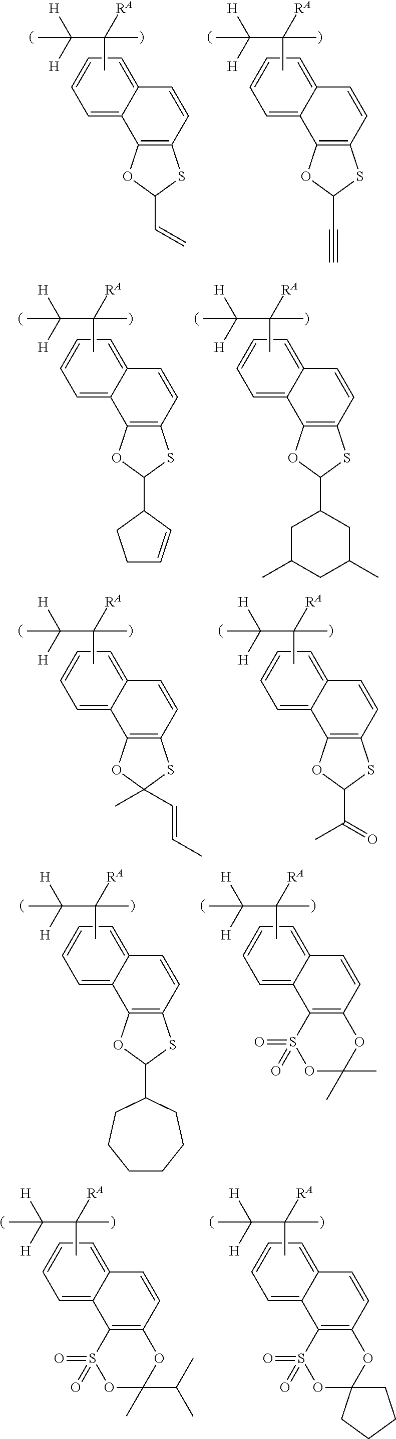

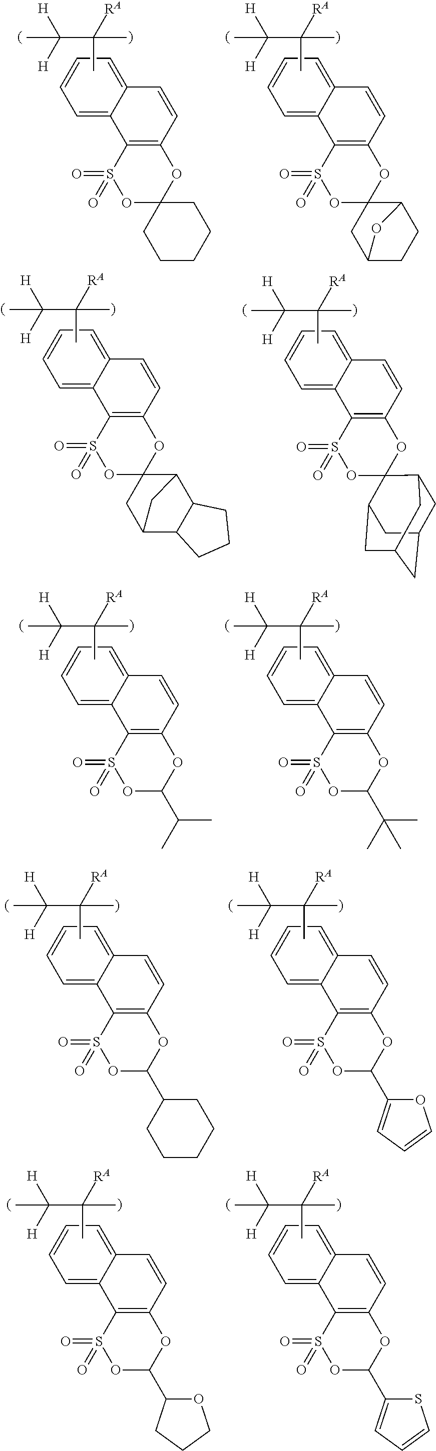

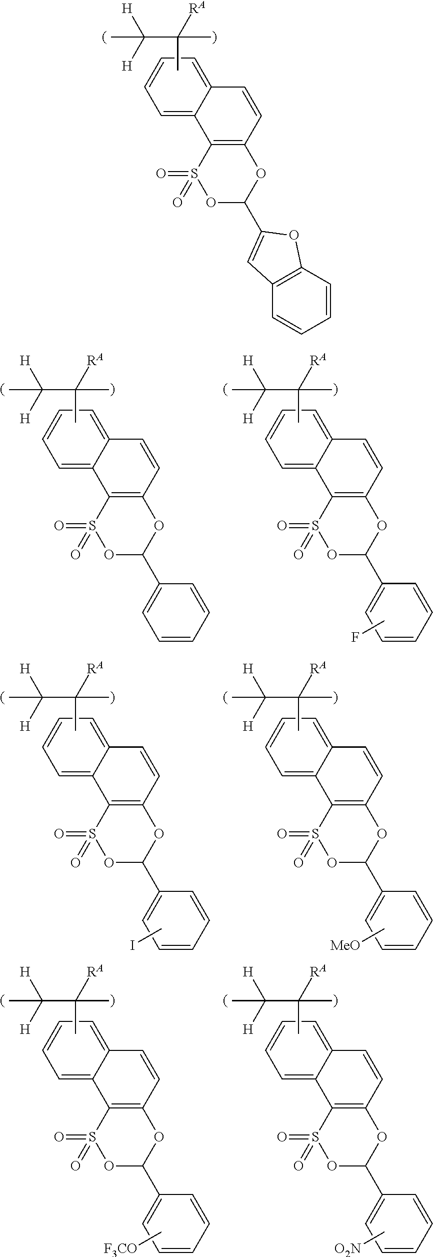

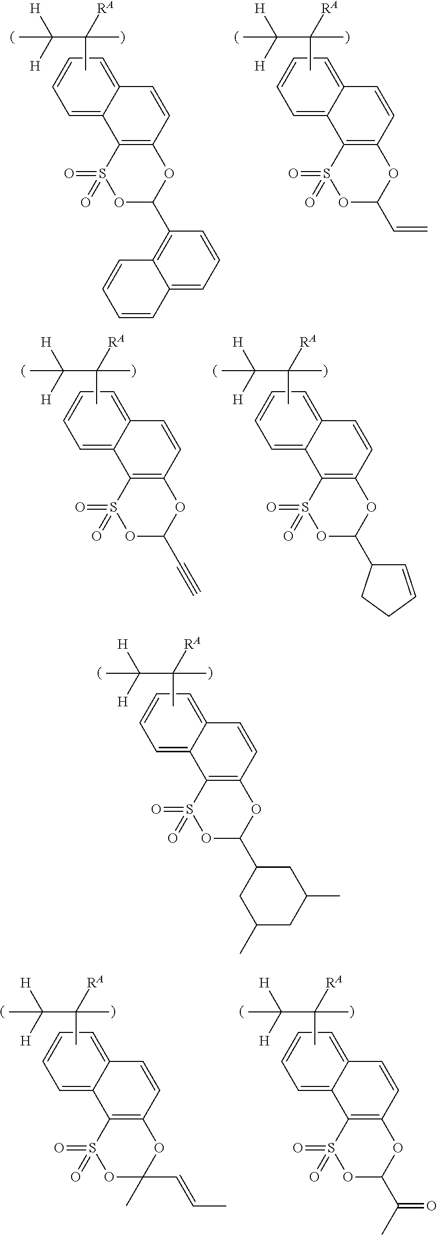

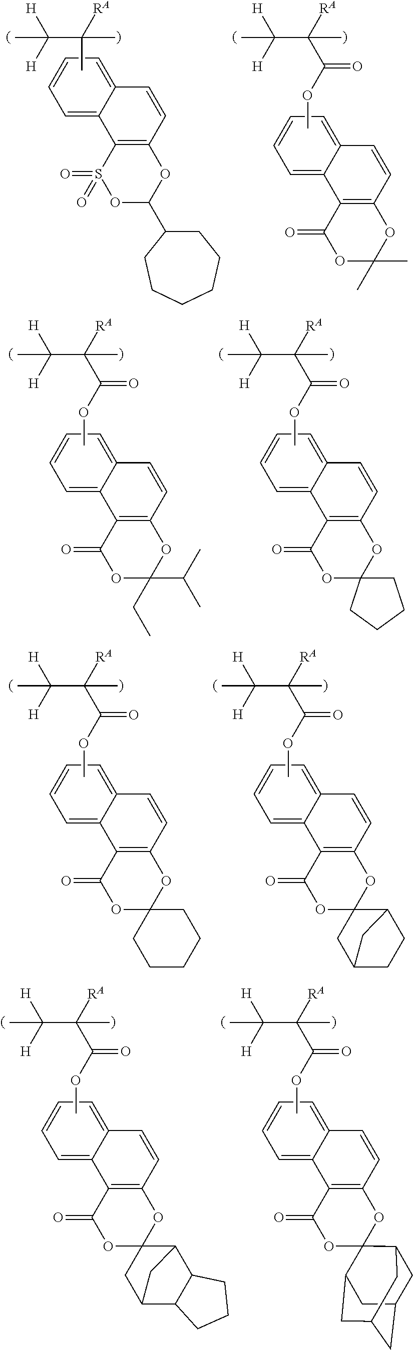

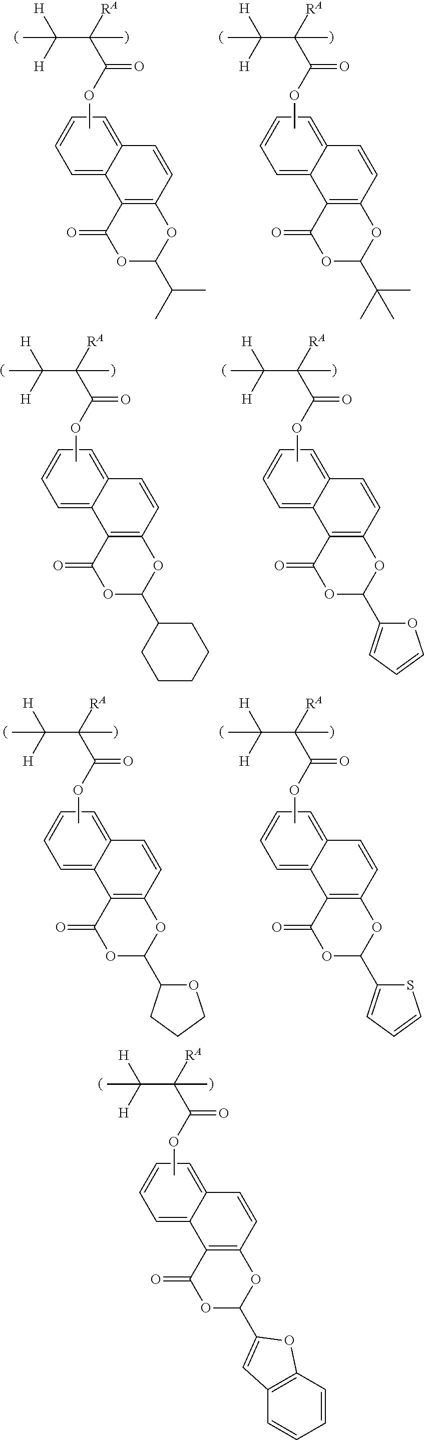

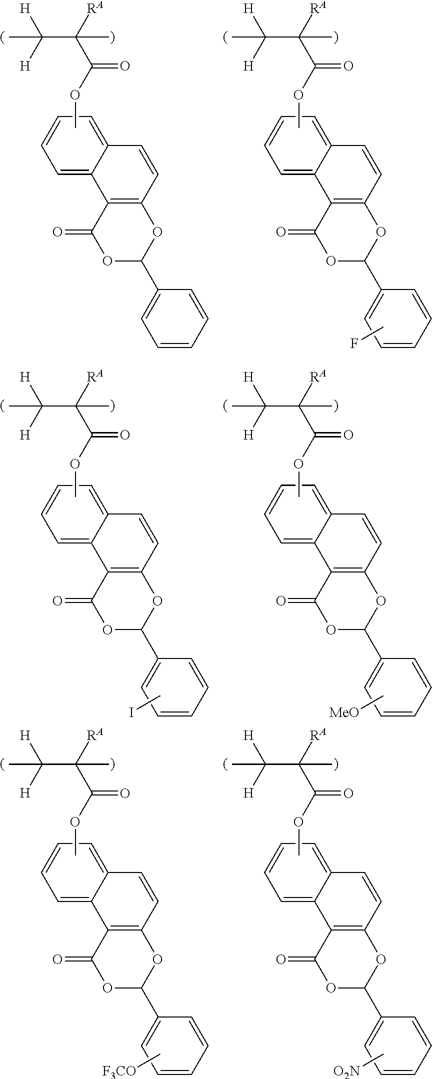

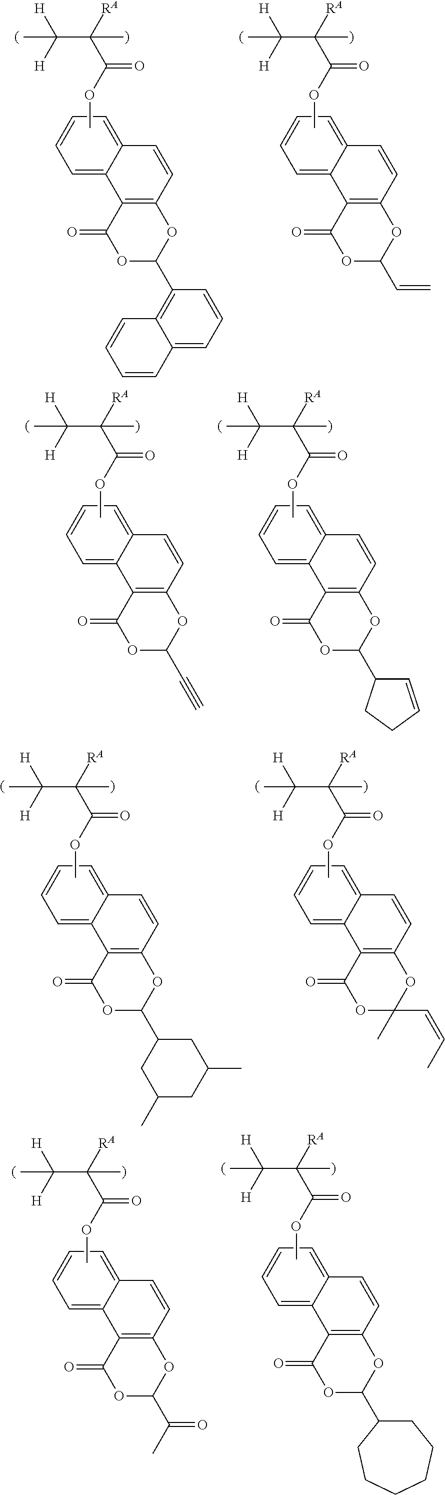

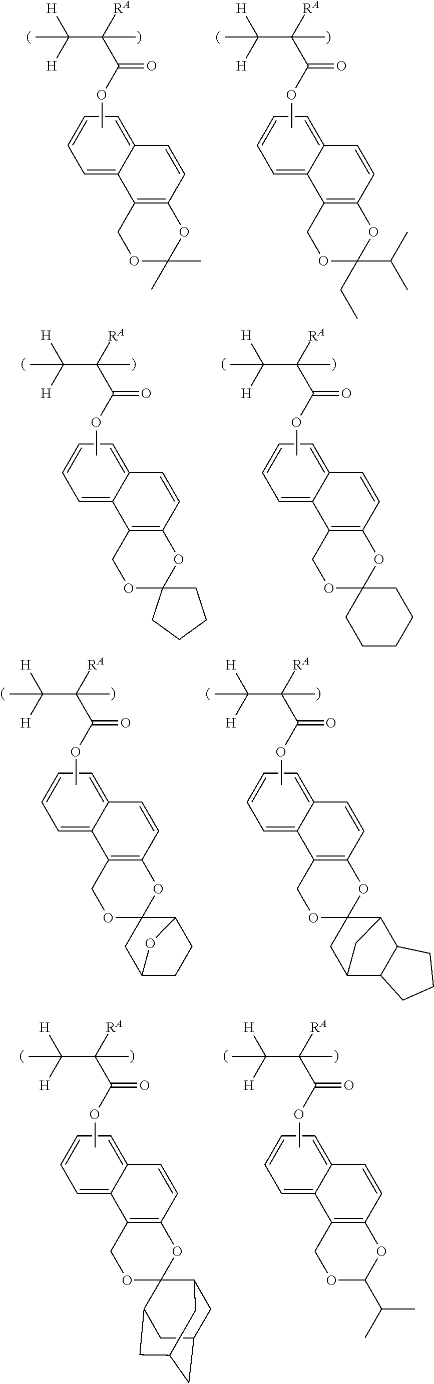

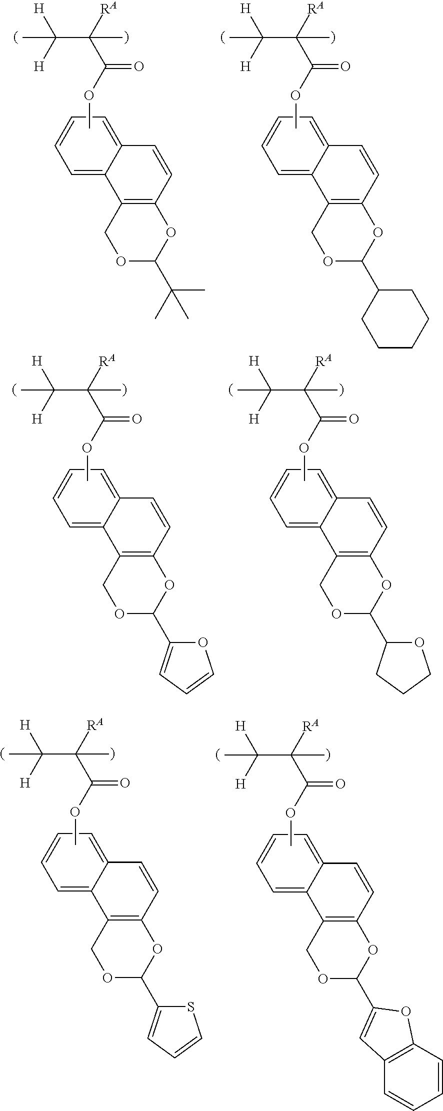

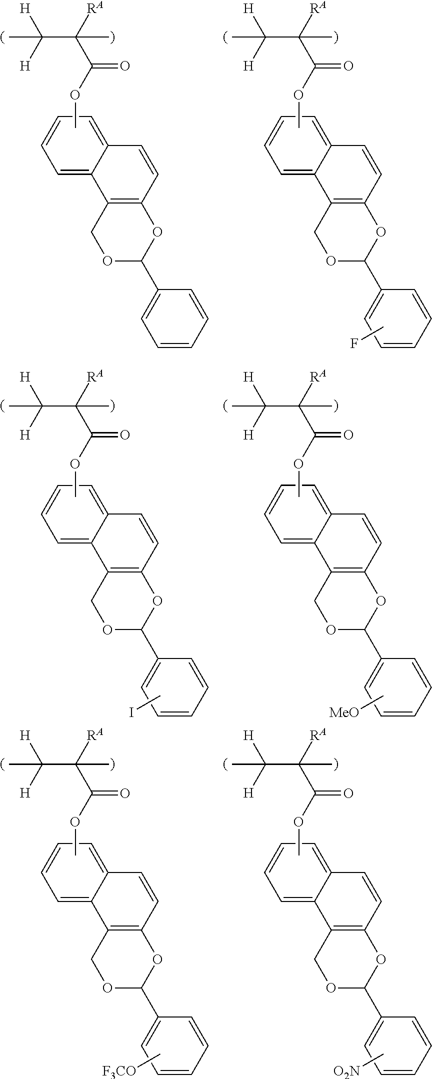

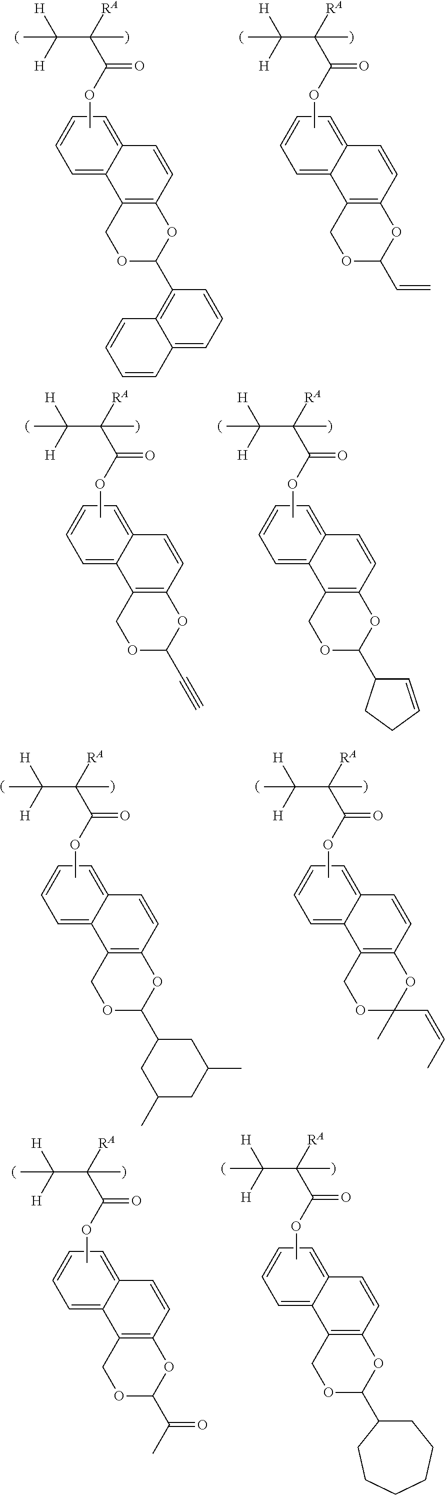

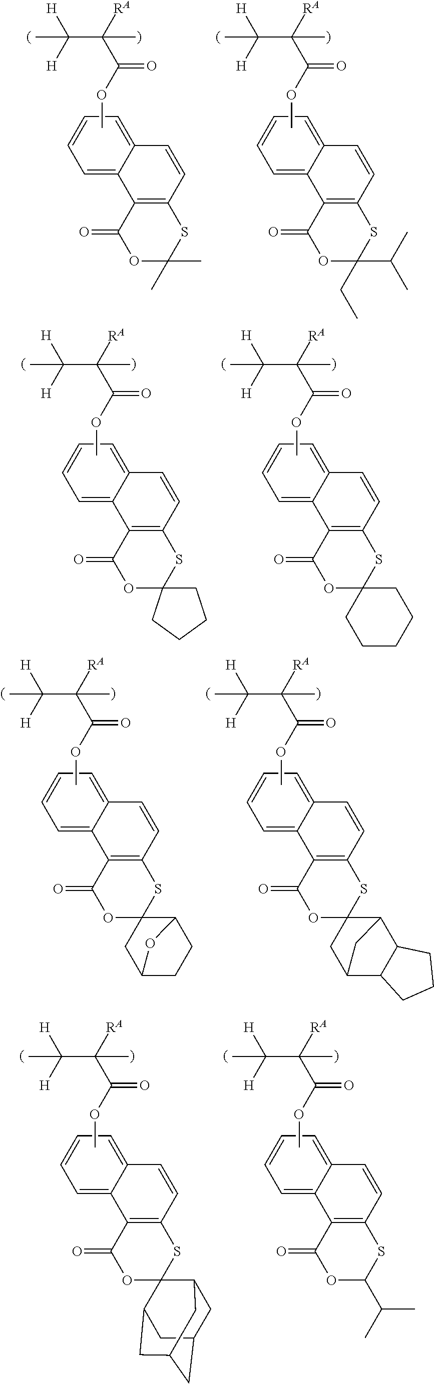

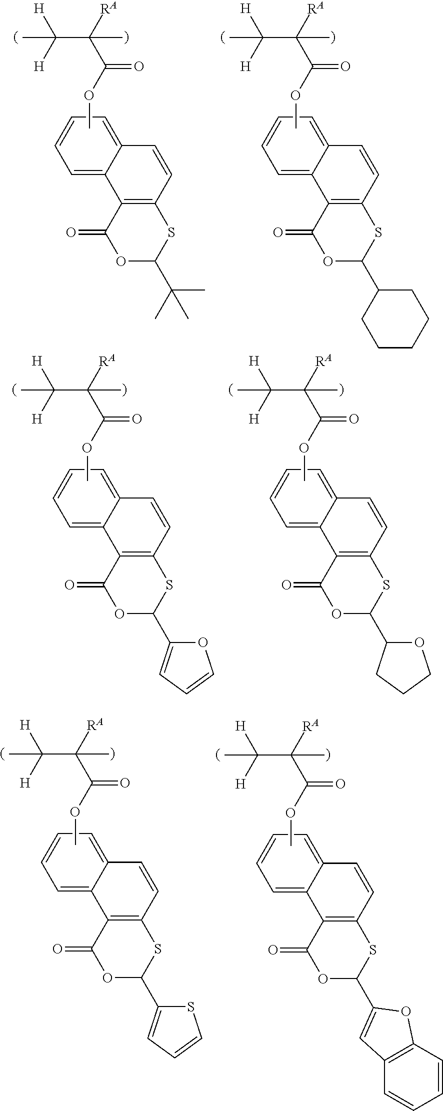

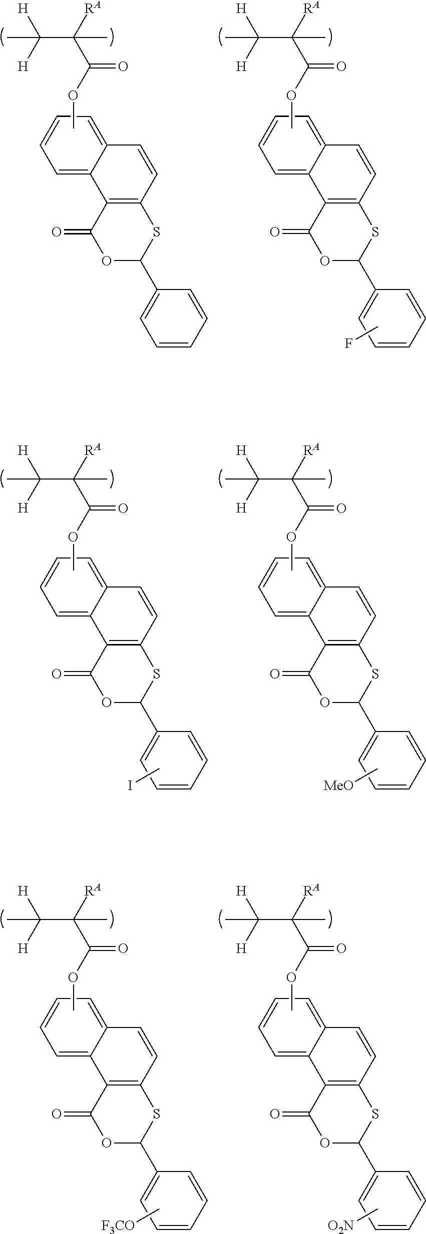

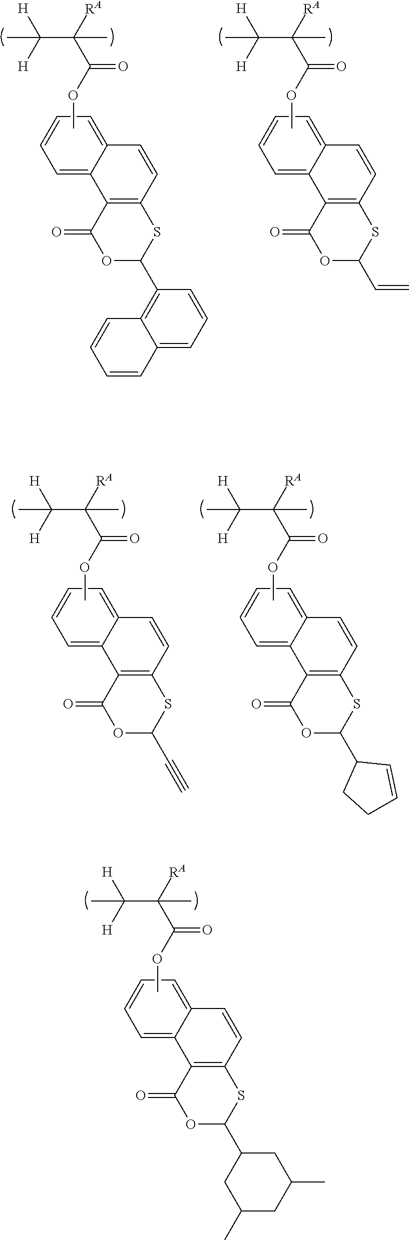

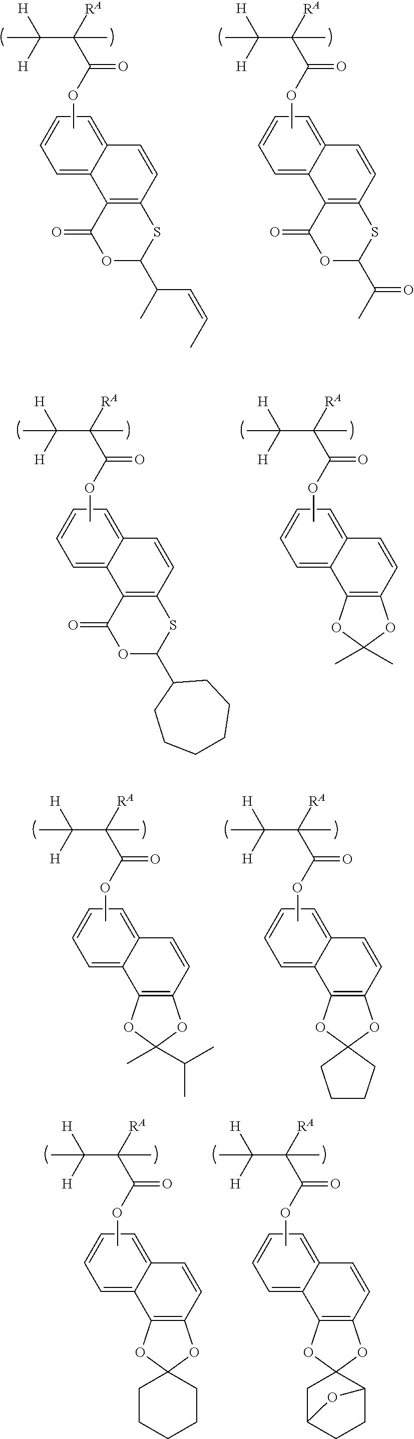

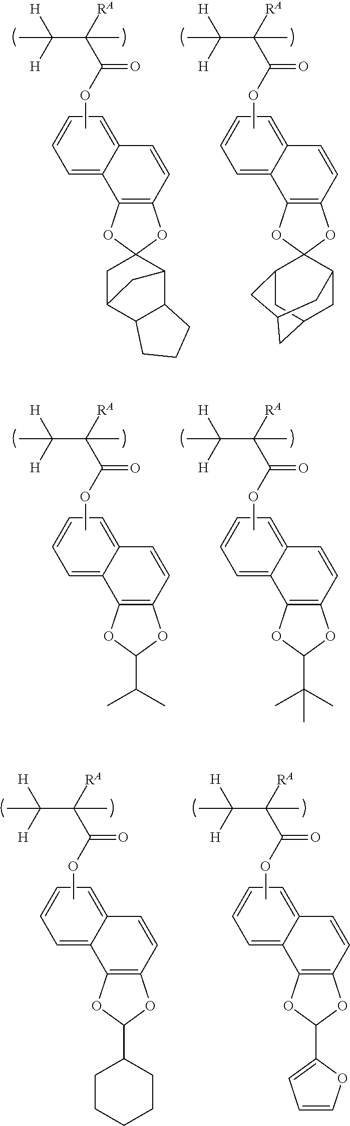

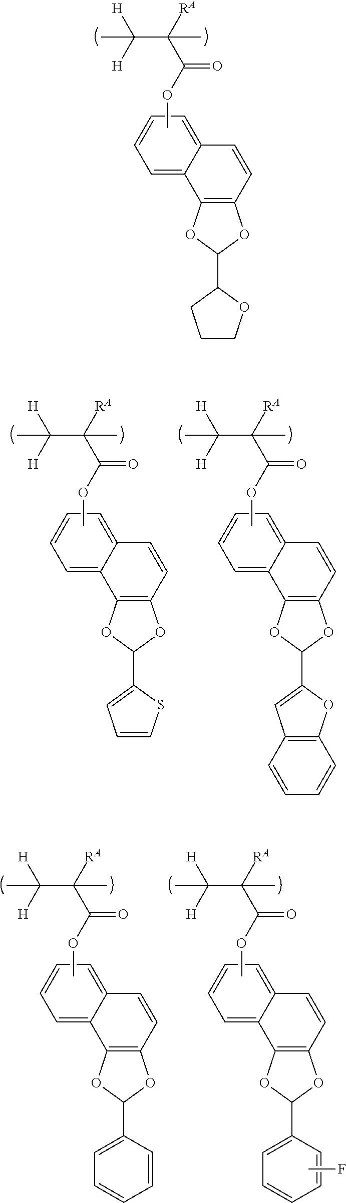

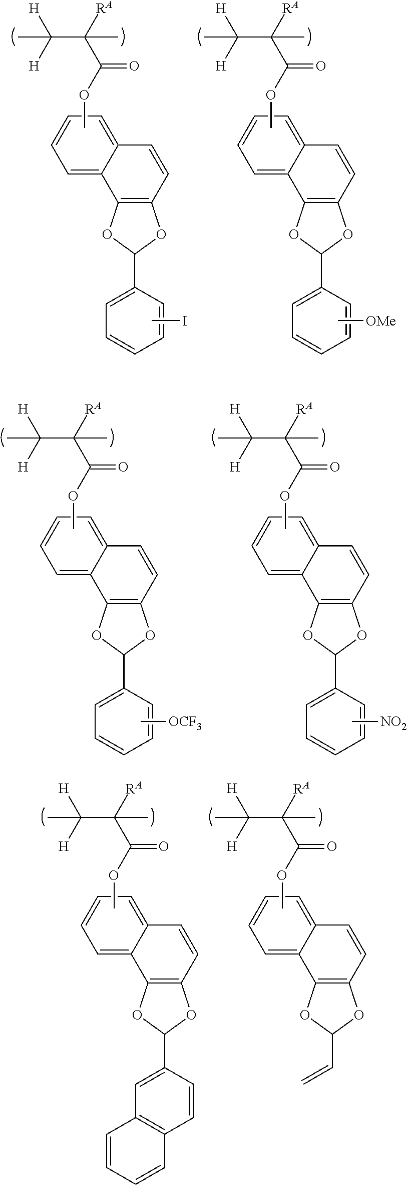

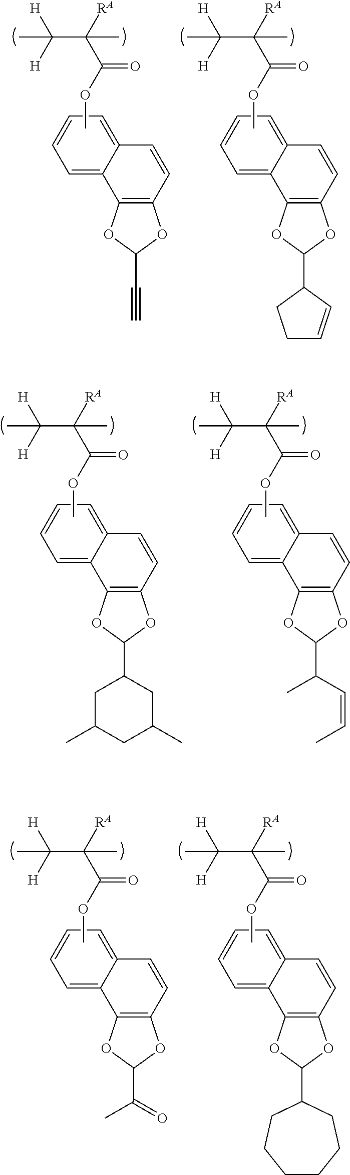

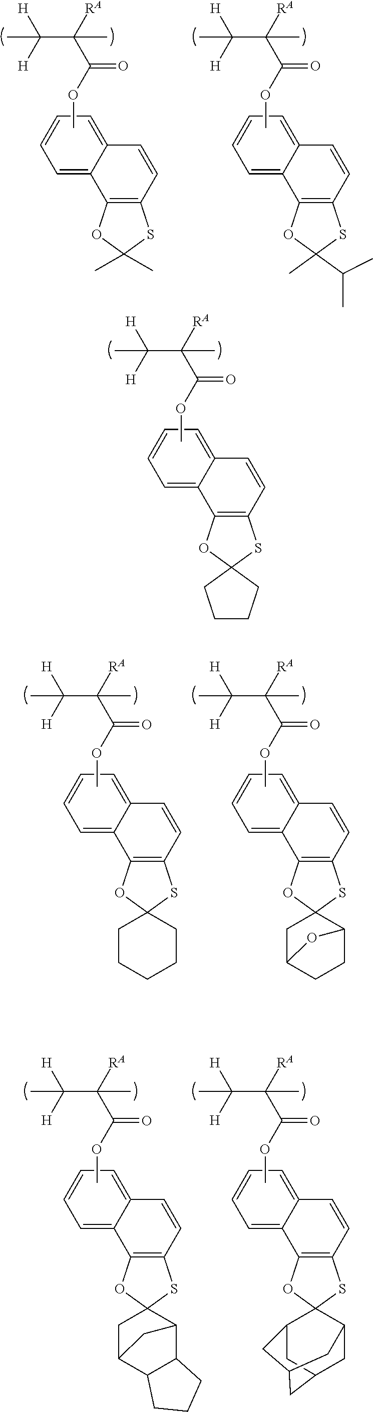

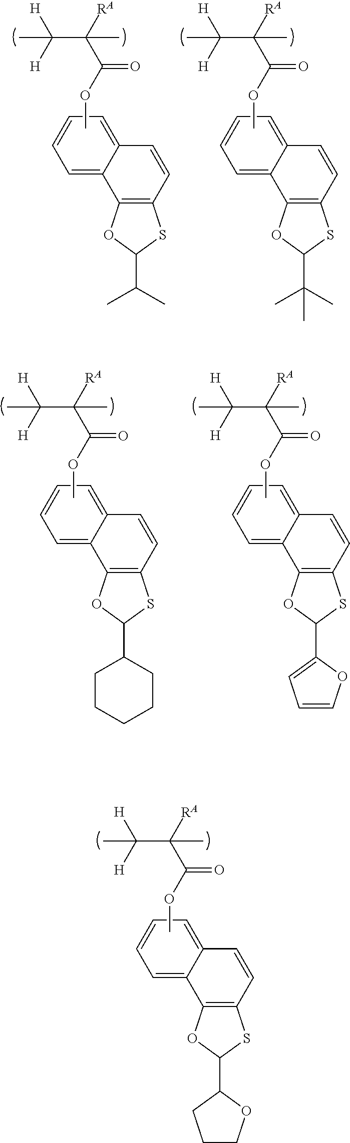

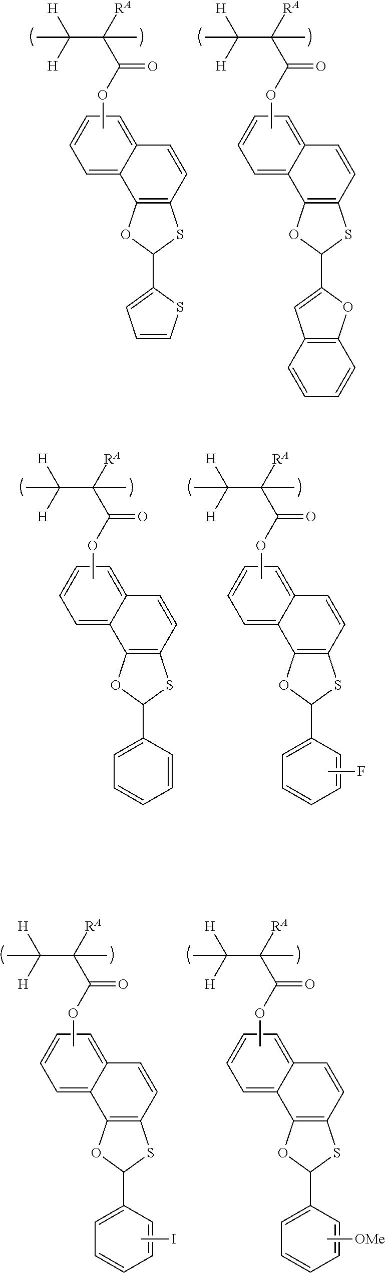

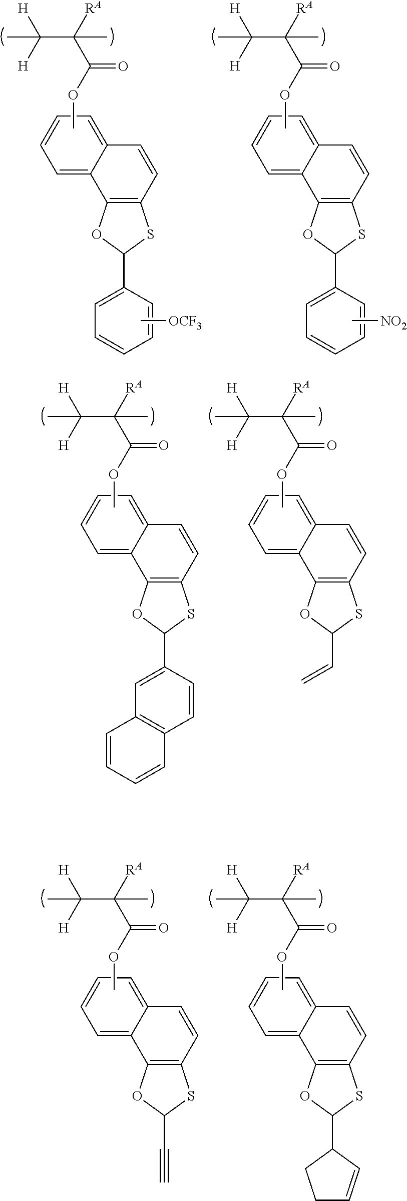

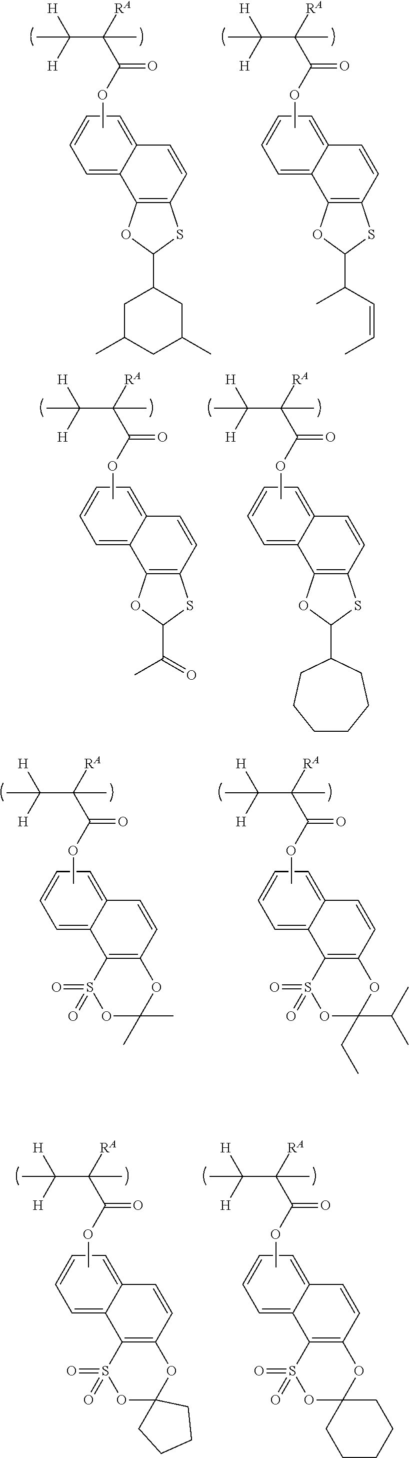

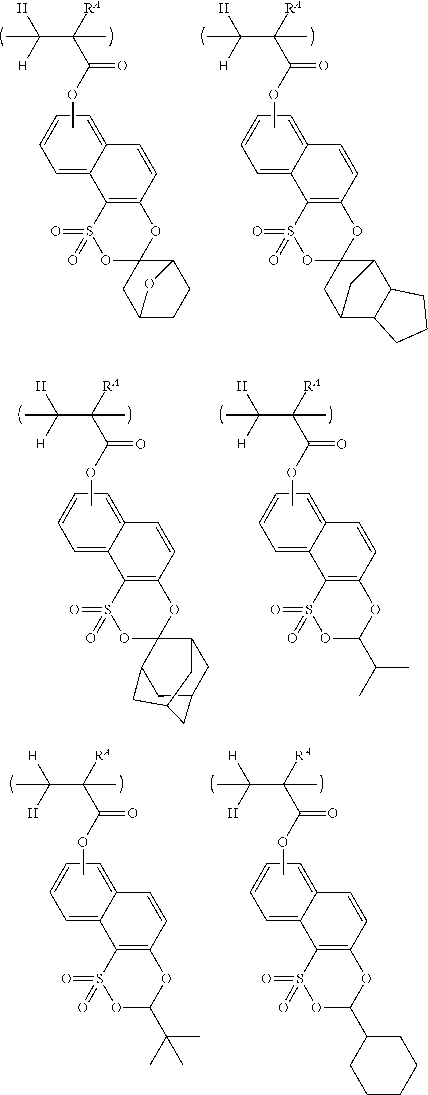

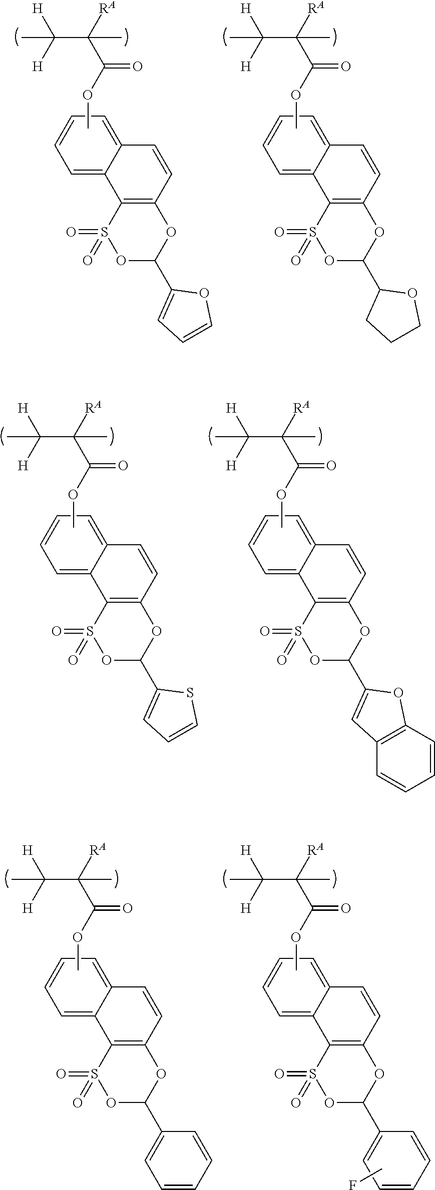

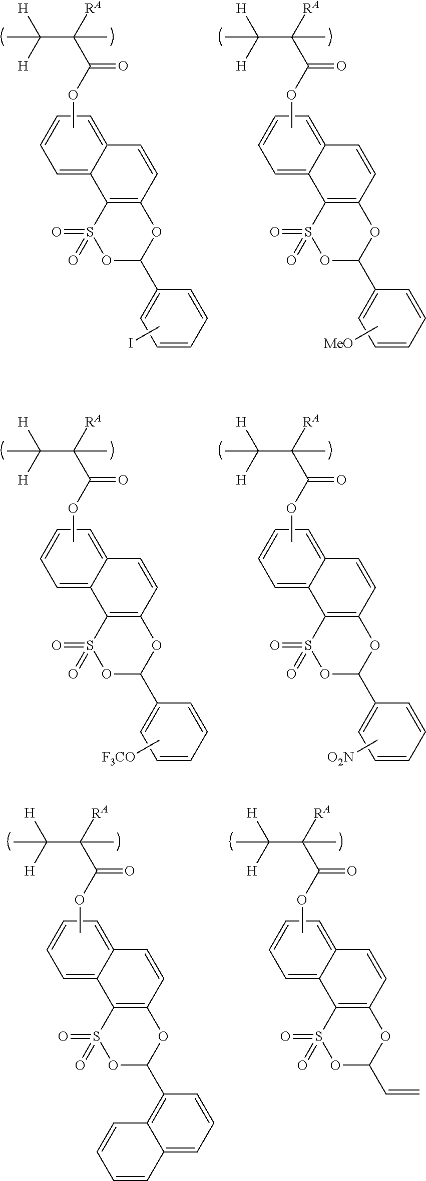

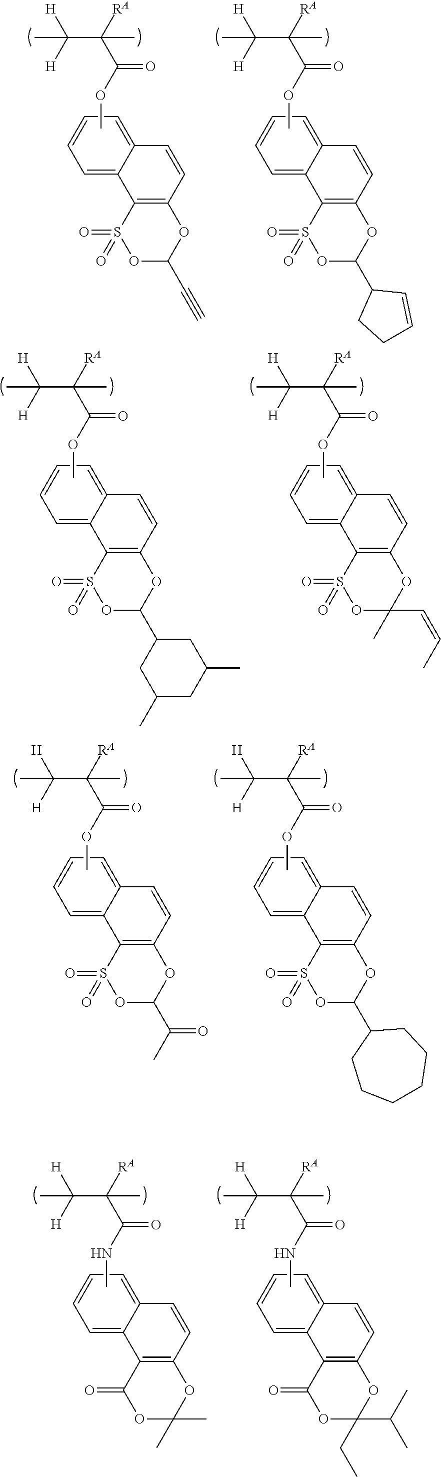

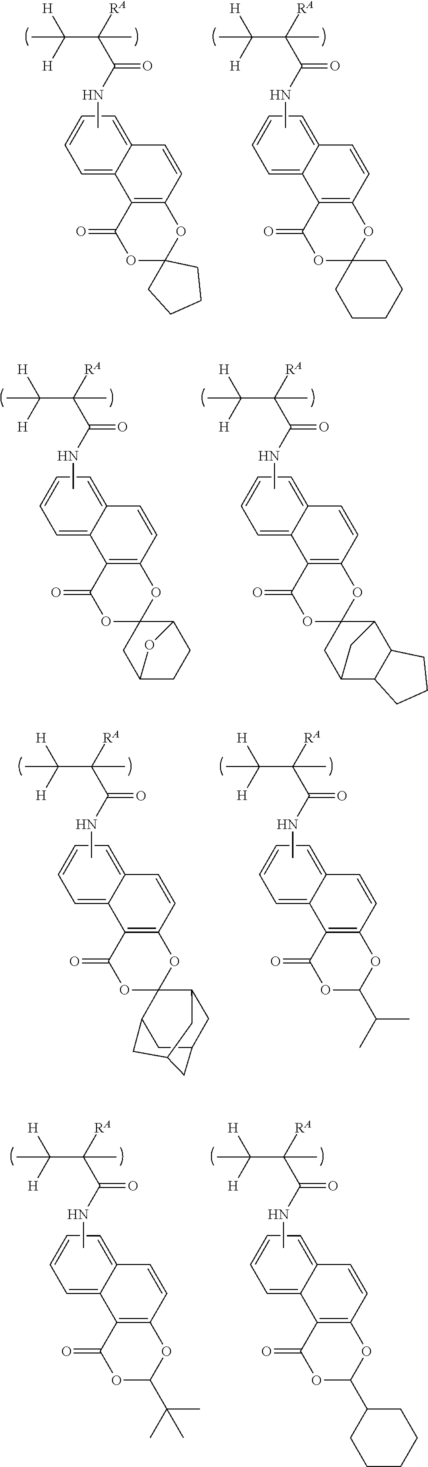

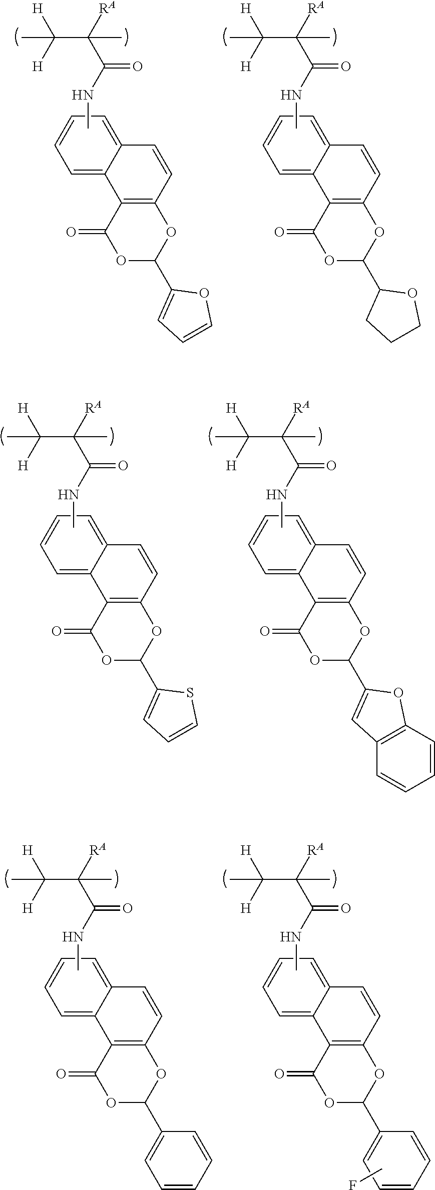

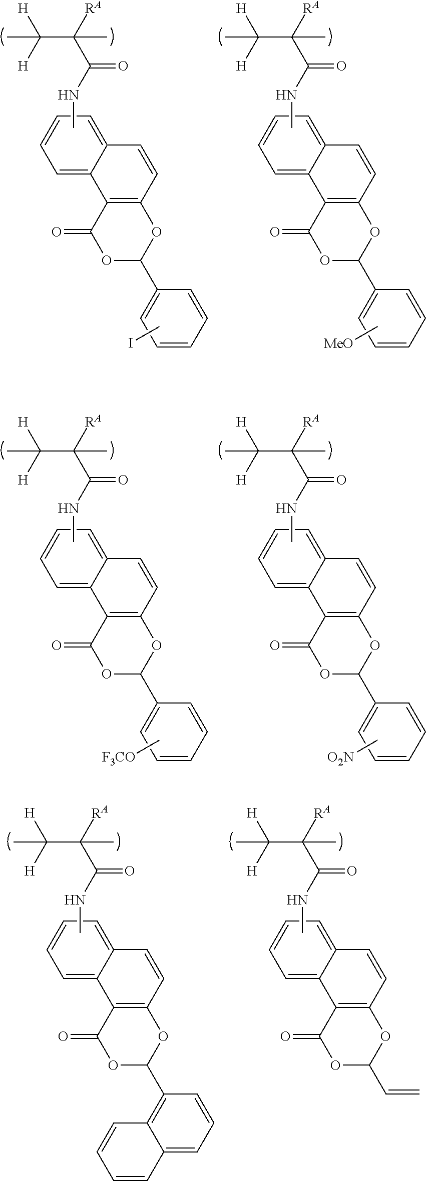

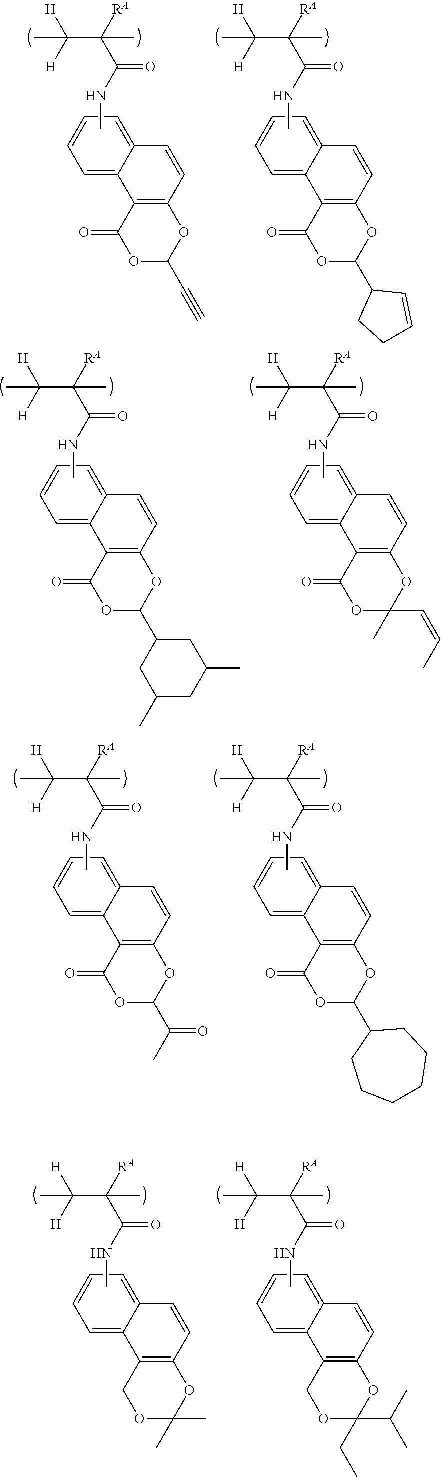

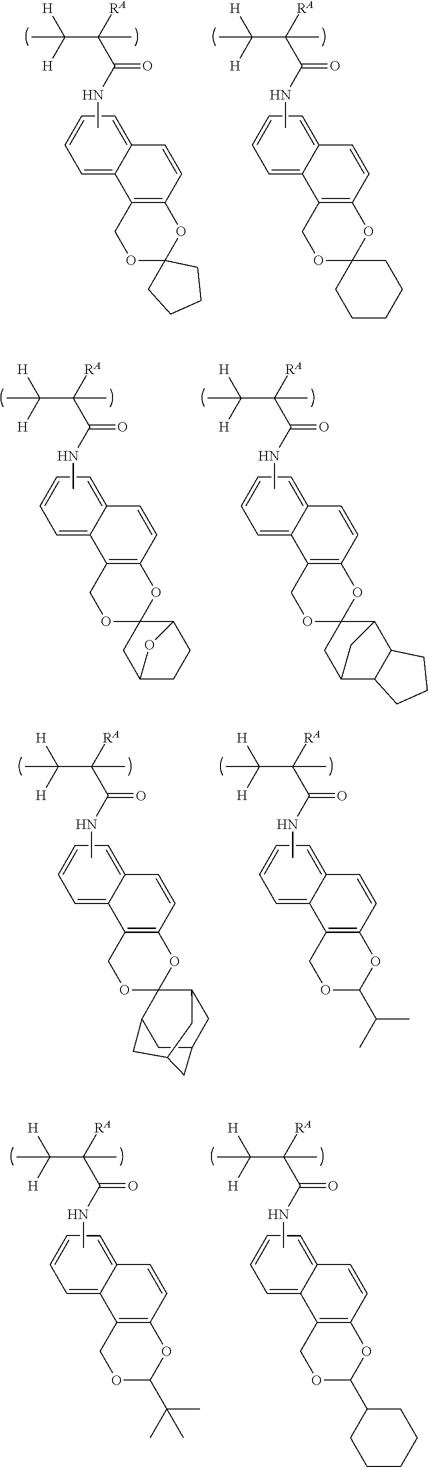

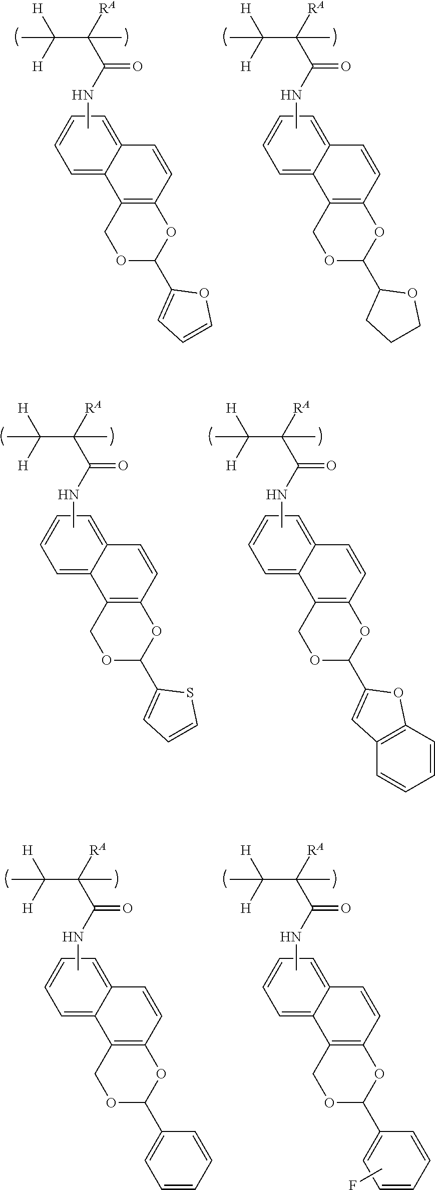

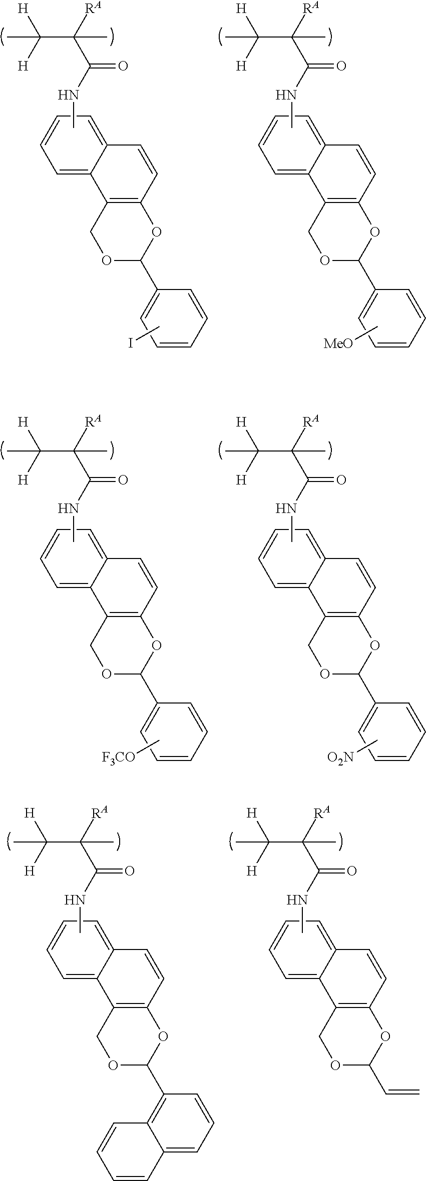

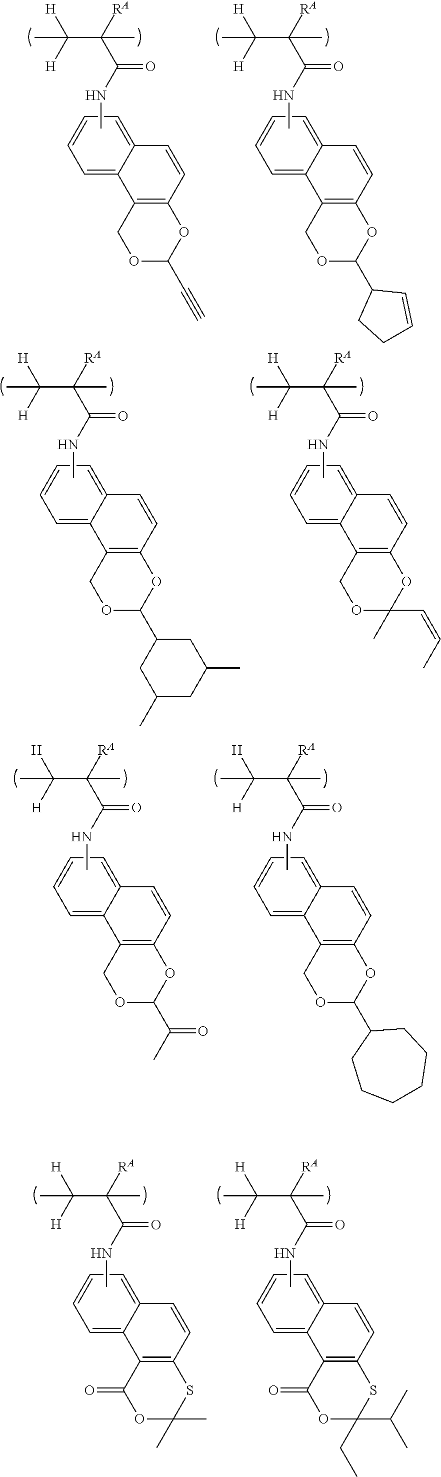

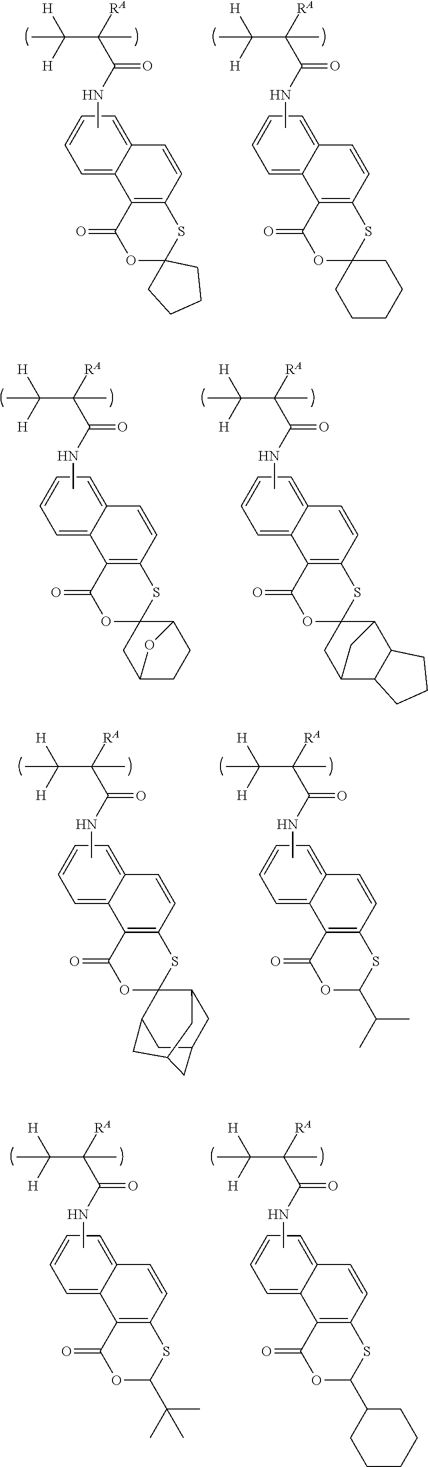

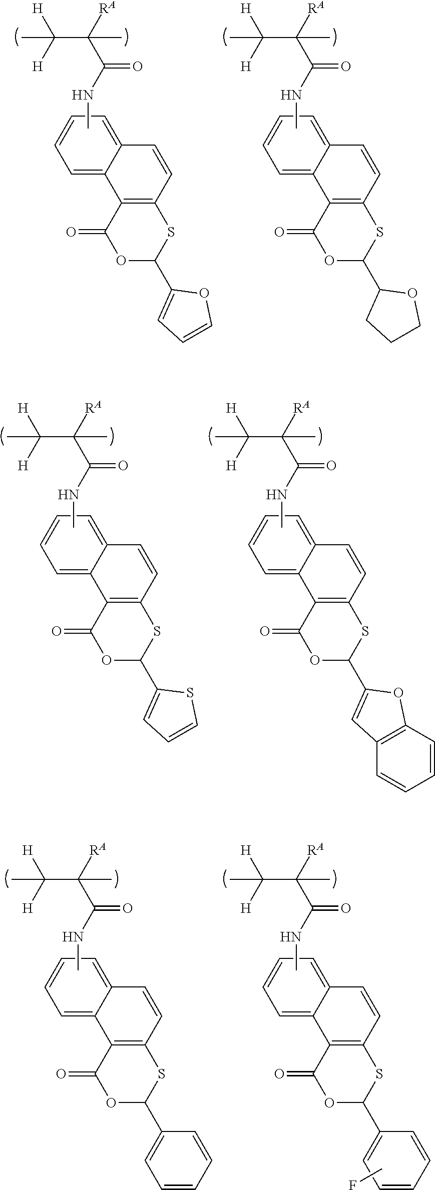

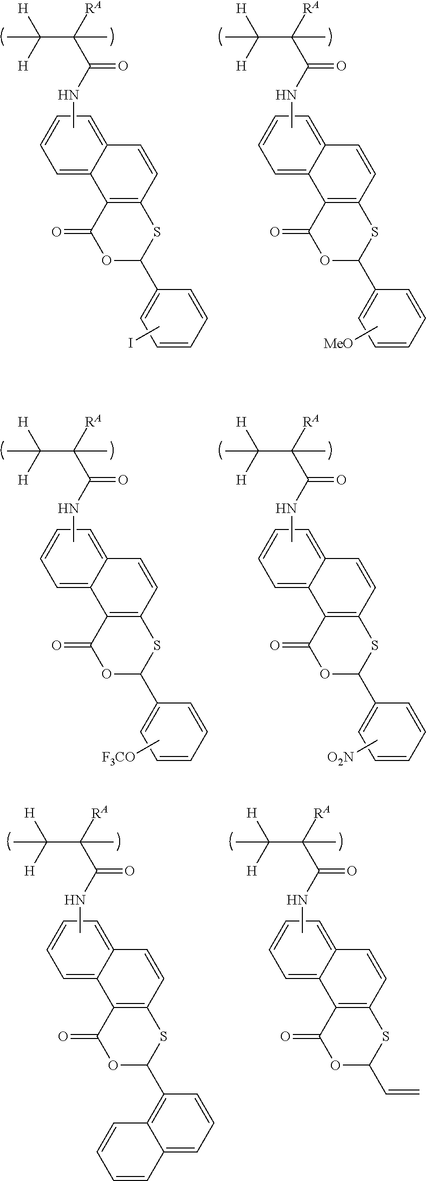

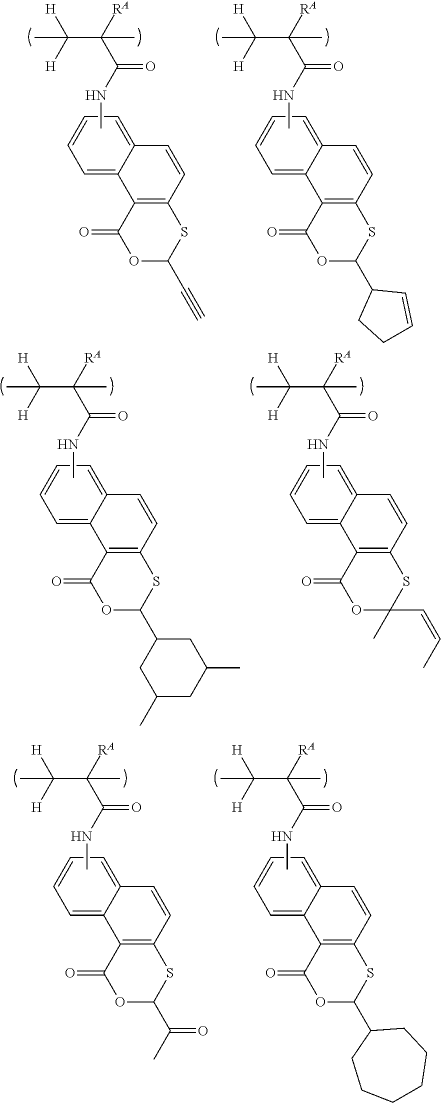

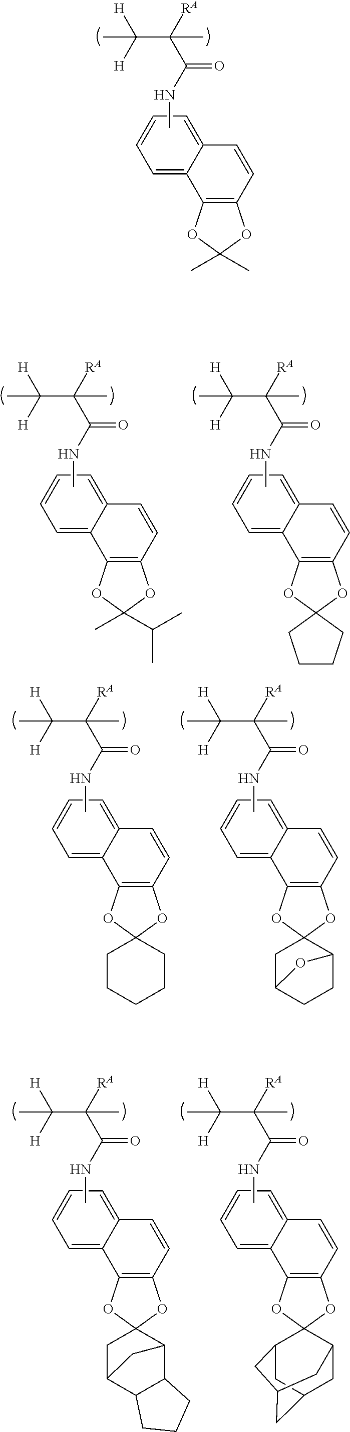

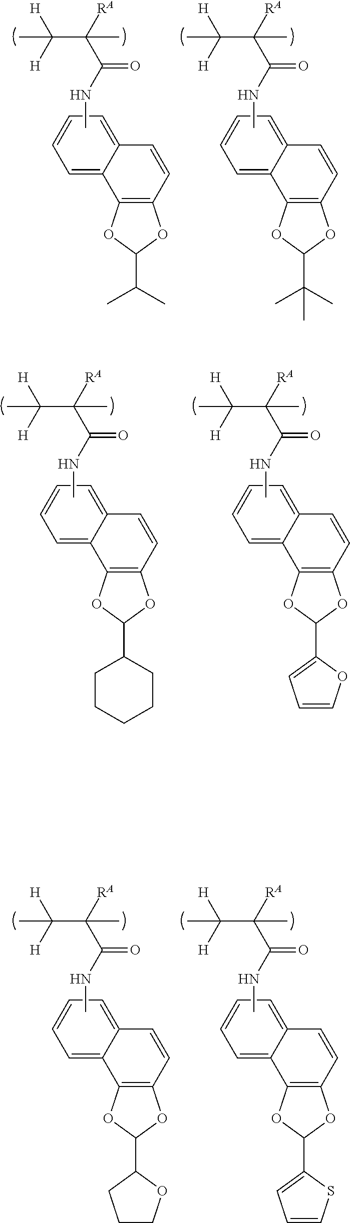

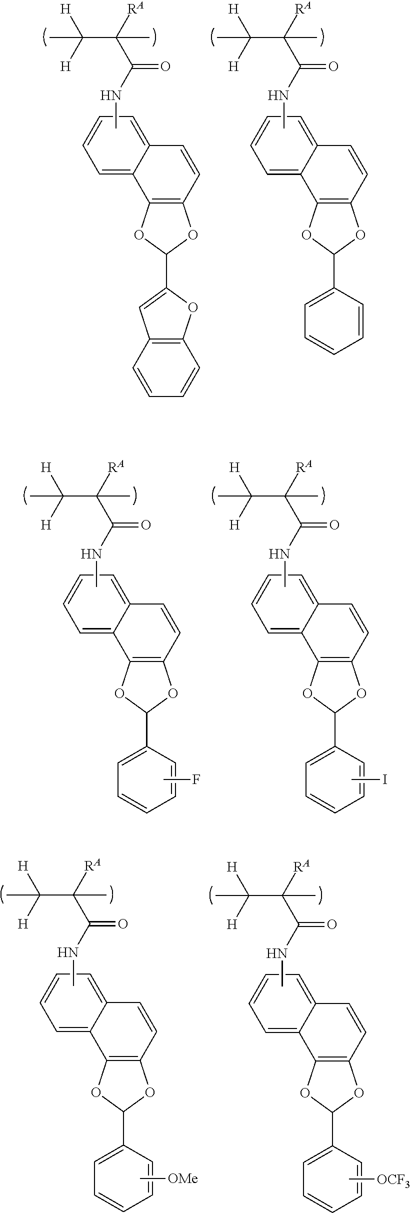

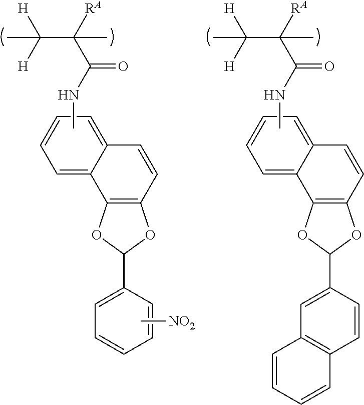

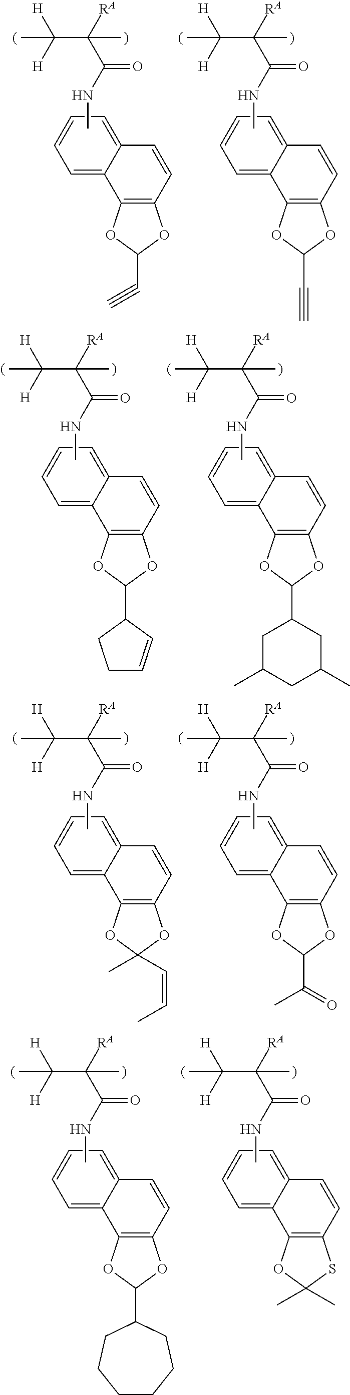

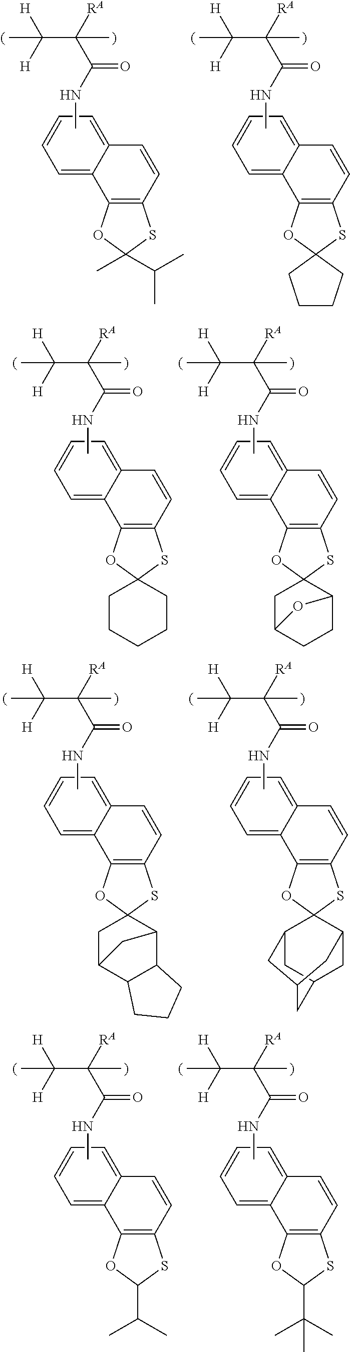

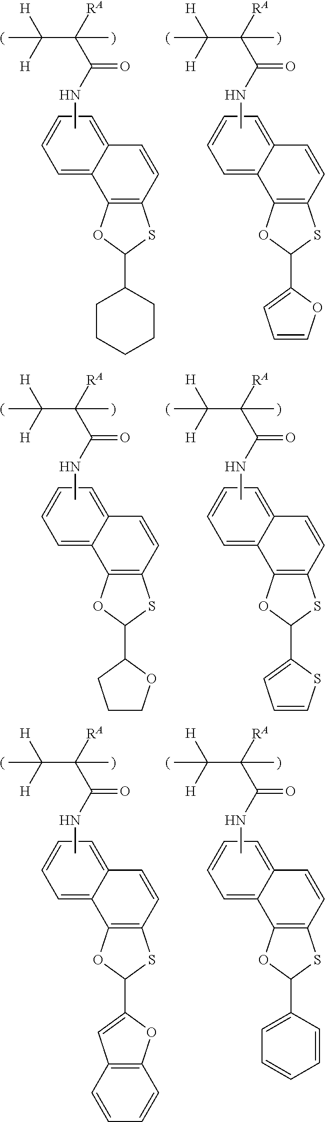

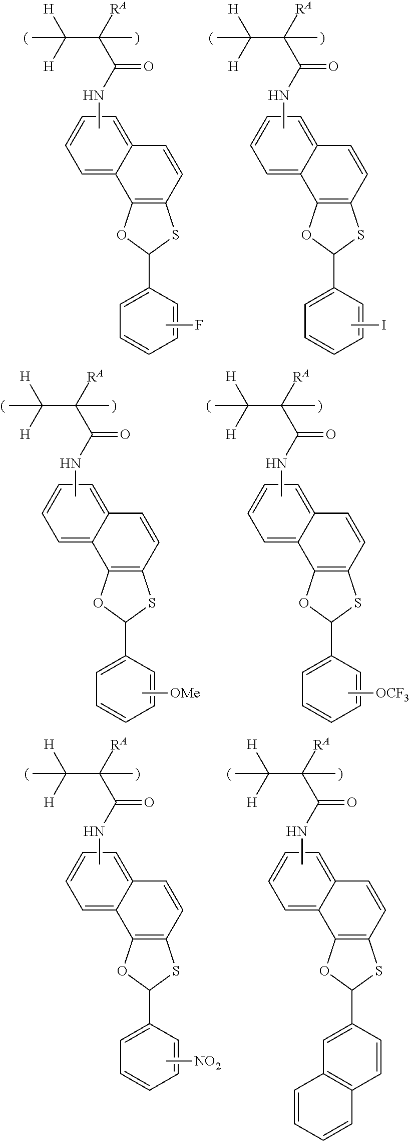

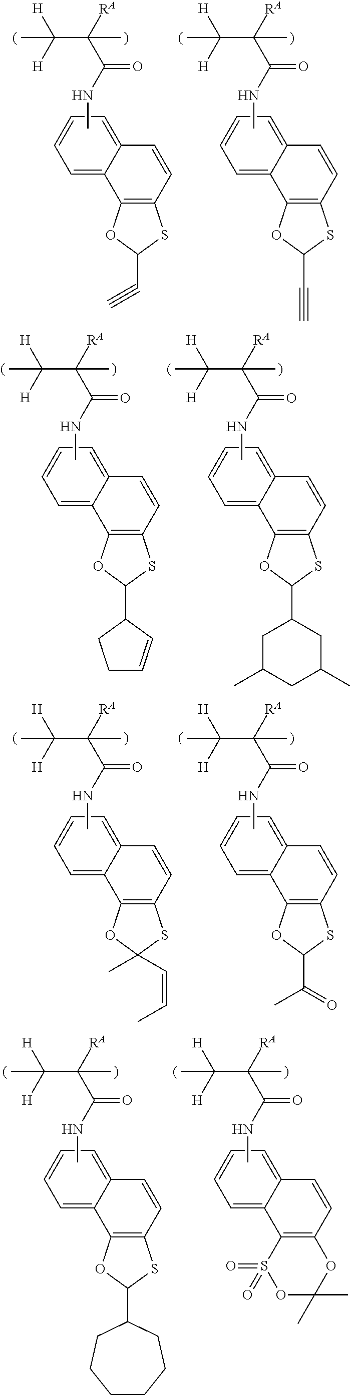

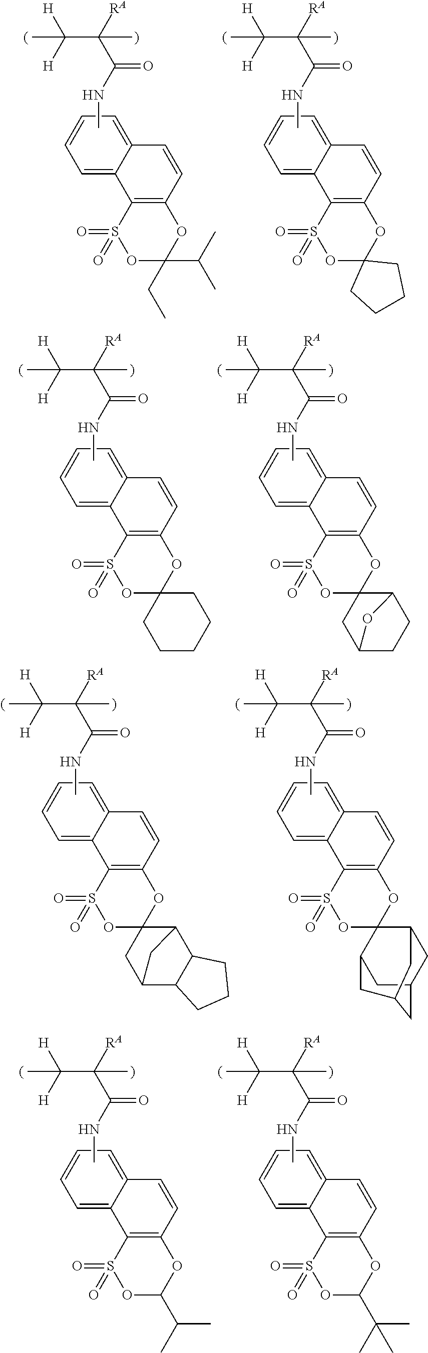

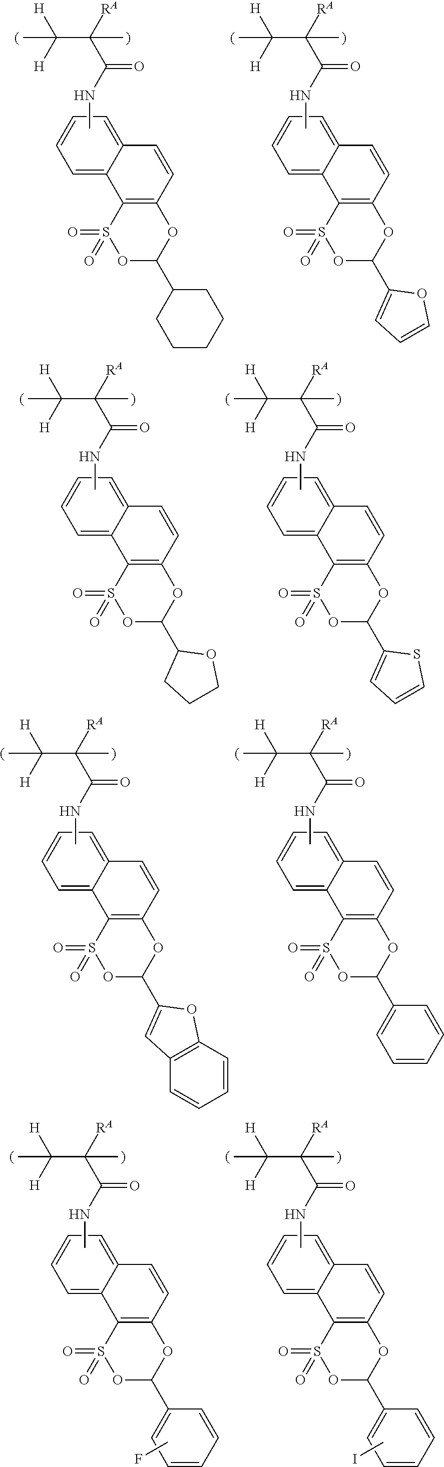

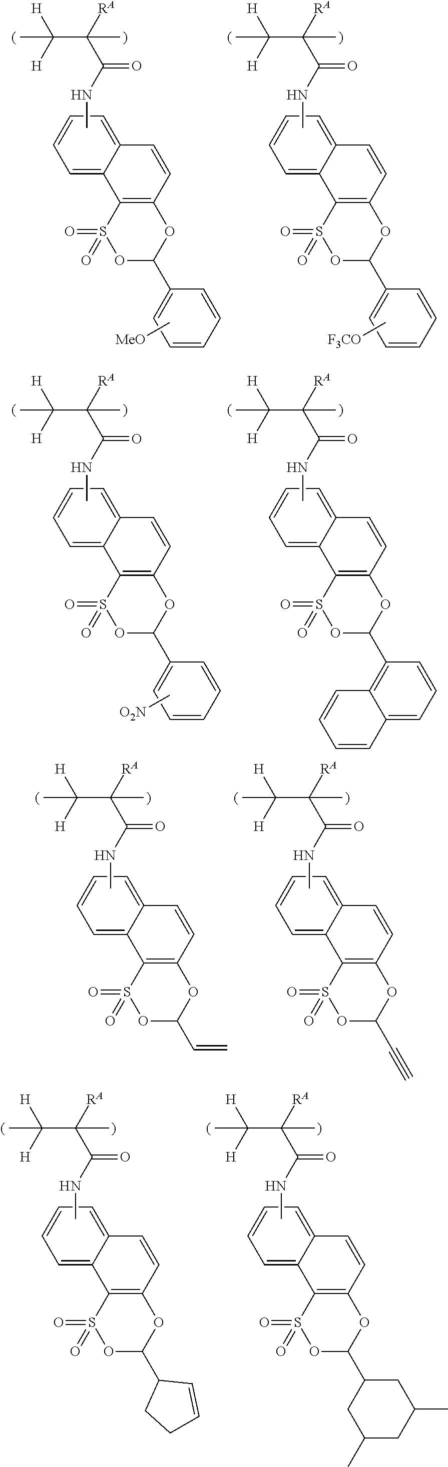

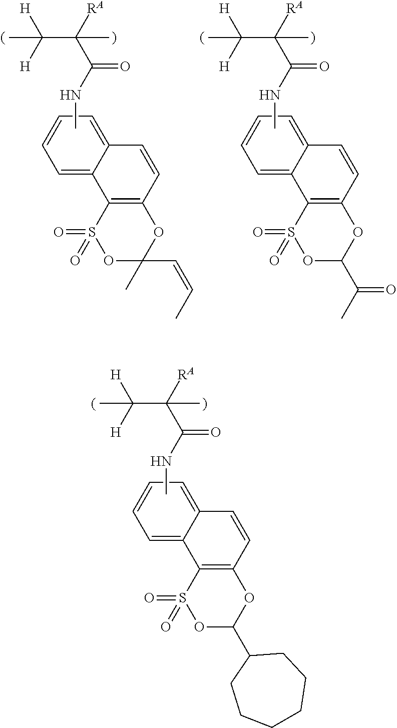

In a preferred embodiment, the polymer further comprises repeat units having the formula (b1) or repeat units having the formula (b2). These units are referred to as repeat units (b1) and (b2), hereinafter.

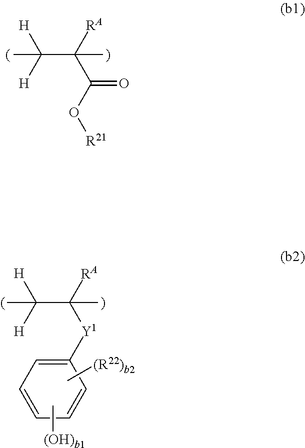

In formulae (b1) and (b2), R^{A} is each independently hydrogen, fluorine, methyl or trifluoromethyl. Y¹ is a single bond or *—C(═O)—O—, wherein * designates a point of attachment to the carbon atom in the backbone. R²¹ is hydrogen or a C₁-C₂₀ group containing at least one structure selected from hydroxy other than phenolic hydroxy, cyano, carbonyl, carboxy, ether bond, ester bond, sulfonate ester bond, carbonate bond, lactone ring sultone ring, and carboxylic anhydride (—C(═O)—O—C(═O)—). R²² is halogen, carboxy, nitro, cyano, a C₁-C₂₀ hydrocarbyl group which may contain a heteroatom, C₁-C₂₀ hydrocarbyloxy group which may contain a heteroatom, C₂-C₂₀ hydrocarbylcarbonyl group which may contain a heteroatom, C₂-C₂₀ hydrocarbylcarbonyloxy group which may contain a heteroatom, or C₂-C₂₀ hydrocarbyloxycarbonyl group which may contain a heteroatom. When b2 is 2, 3 or 4, a plurality of R²² may be identical or different. The subscript bl is 1, 2, 3 or 4, b2 is 0, 1, 2, 3 or 4, and 1≤b1+b2≤5.

Examples of the repeat unit (bl) are shown below, but not limited thereto. Herein R^{A} is as defined above.

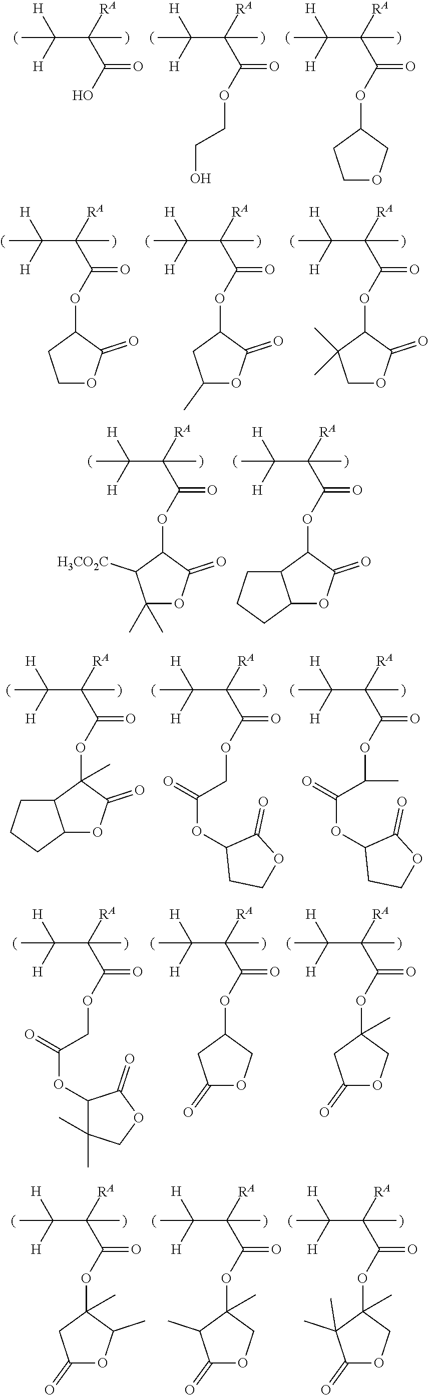

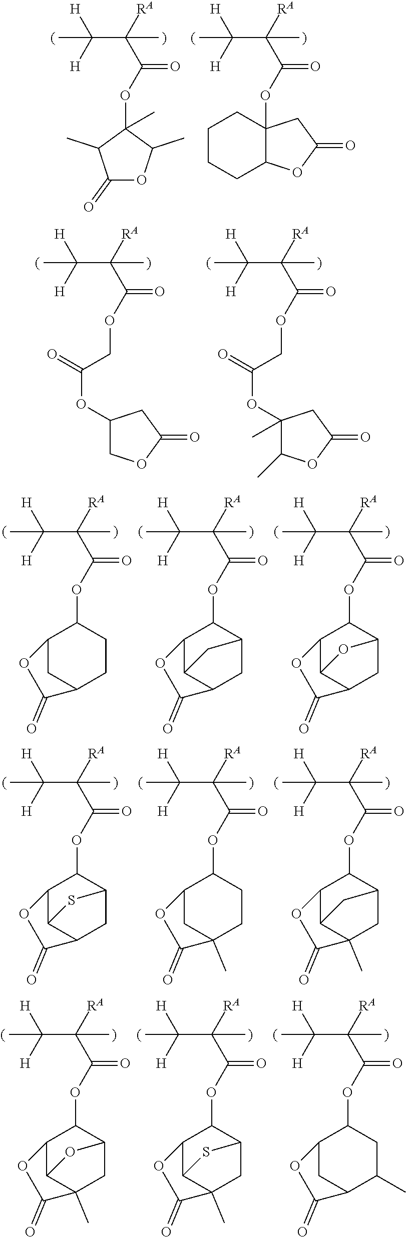

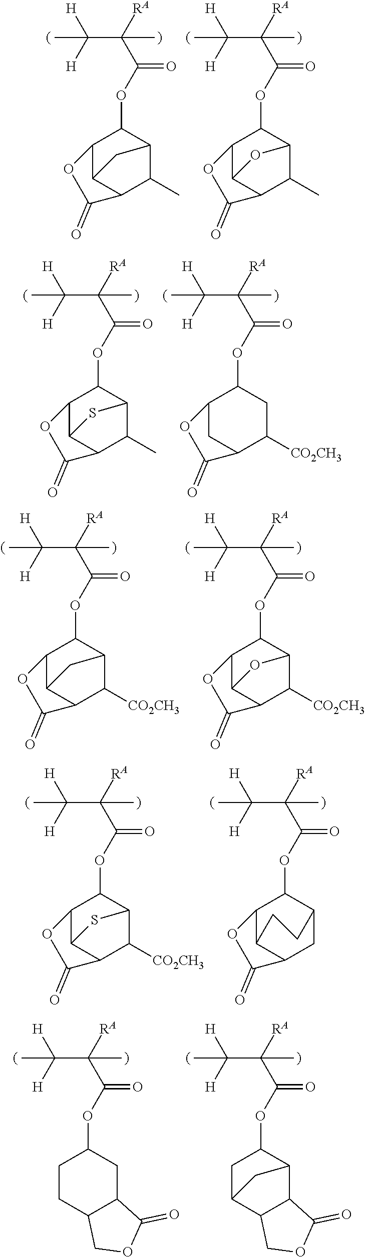

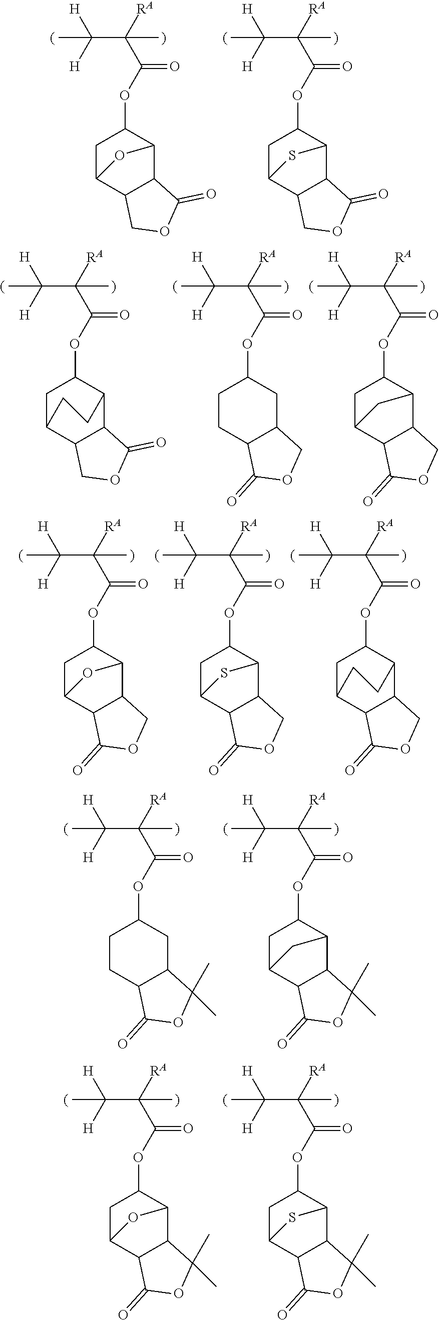

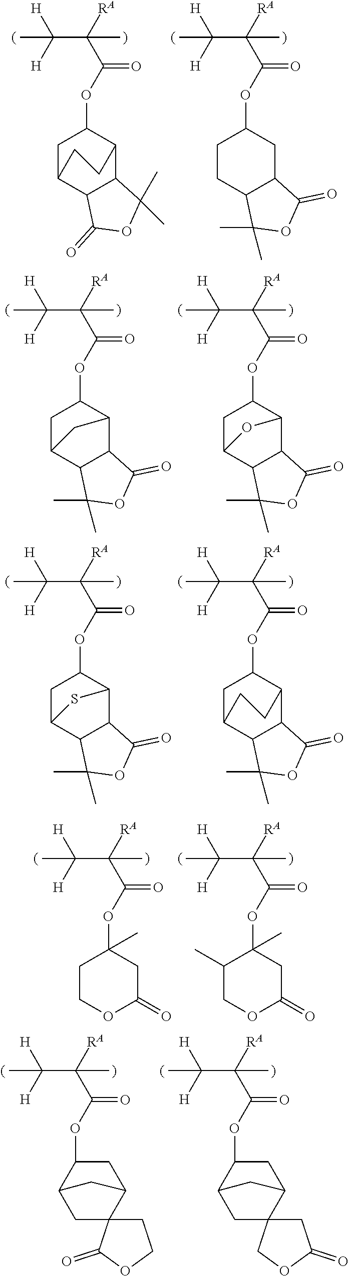

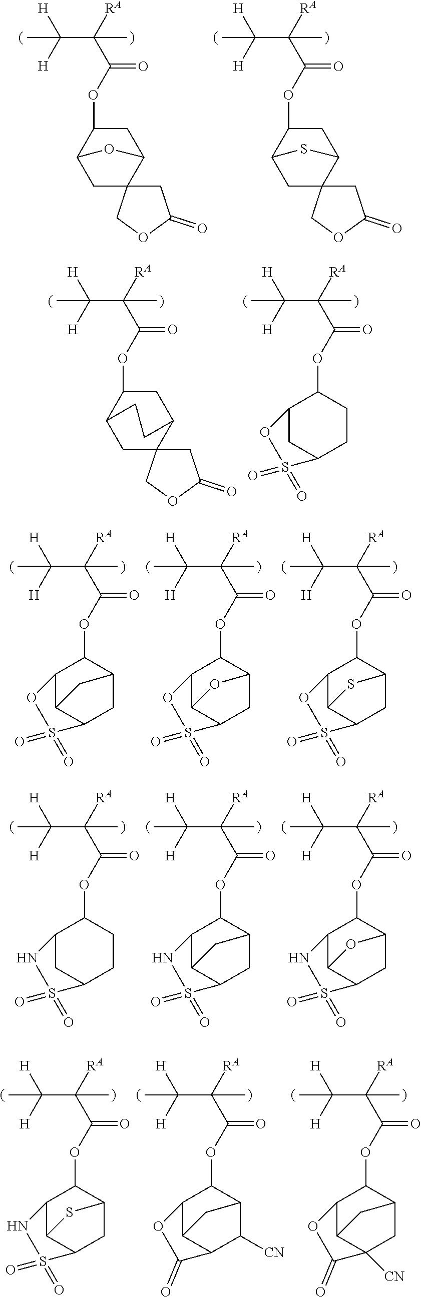

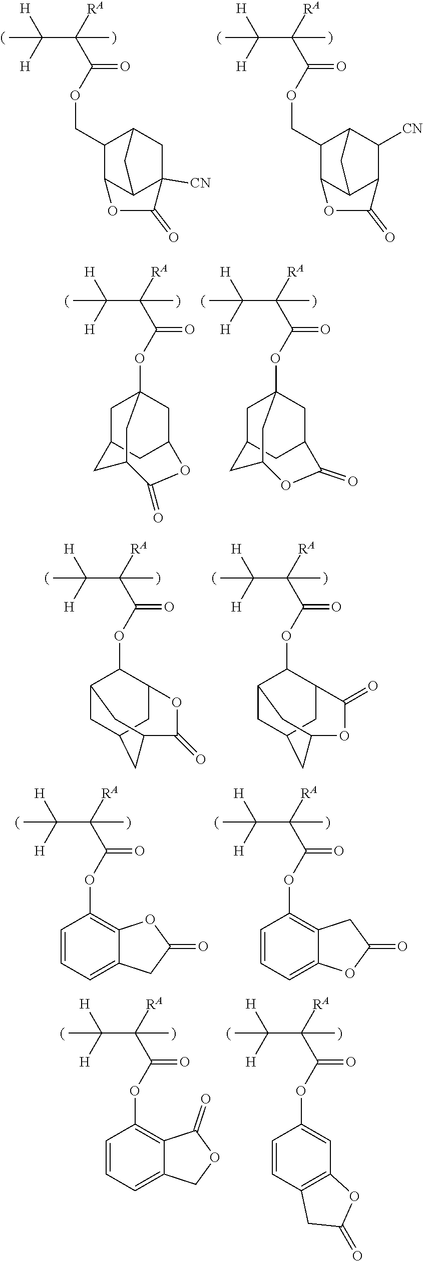

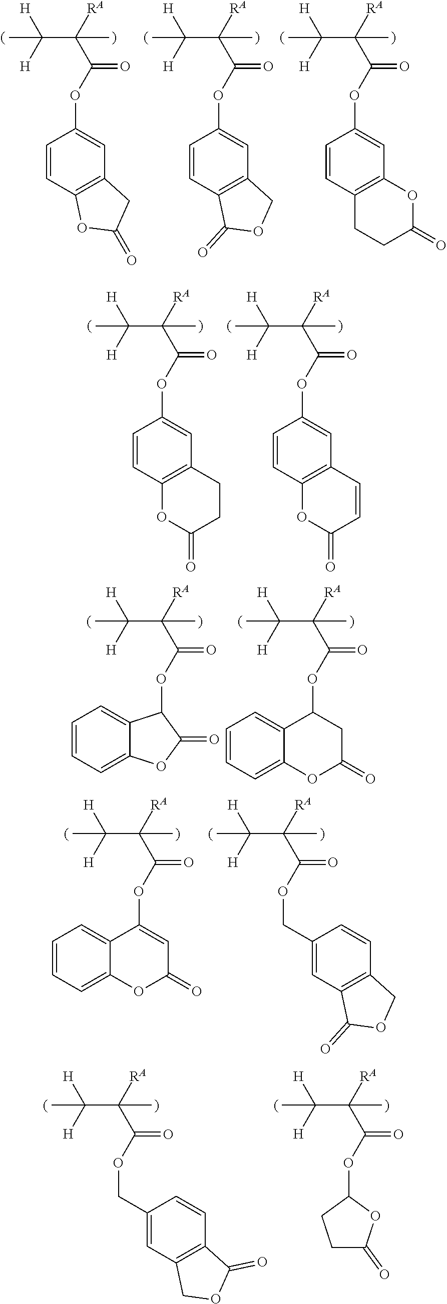

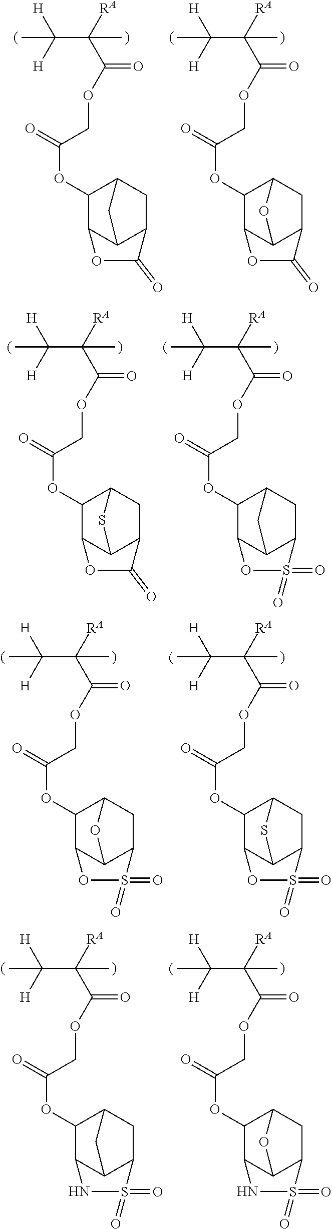

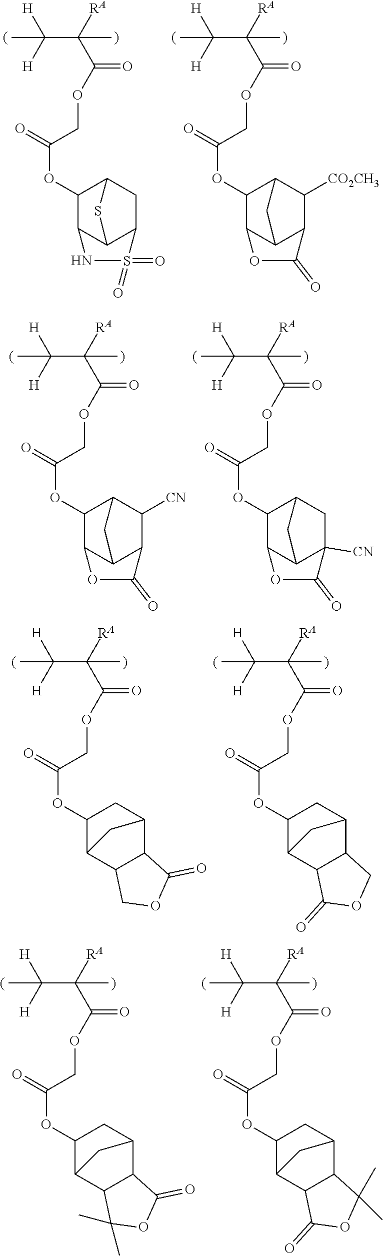

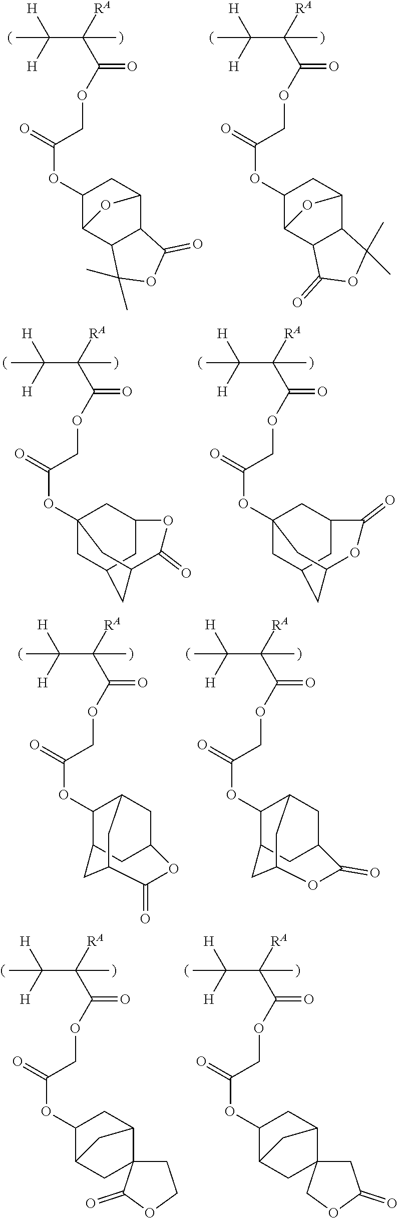

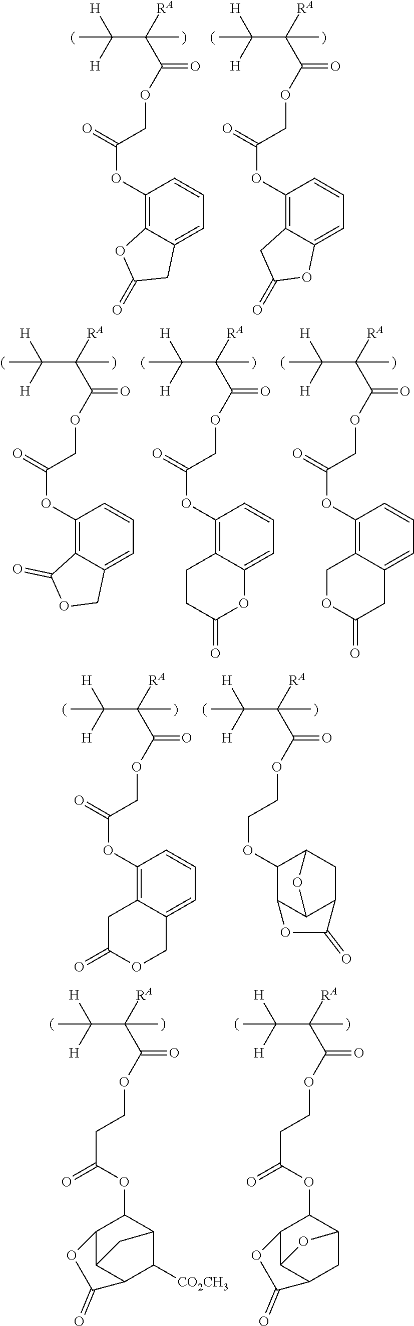

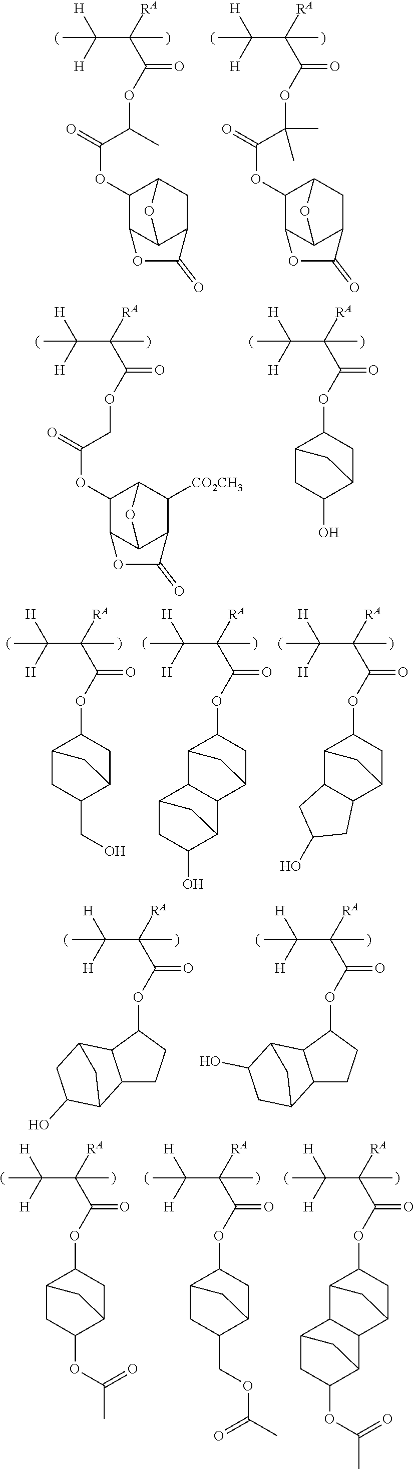

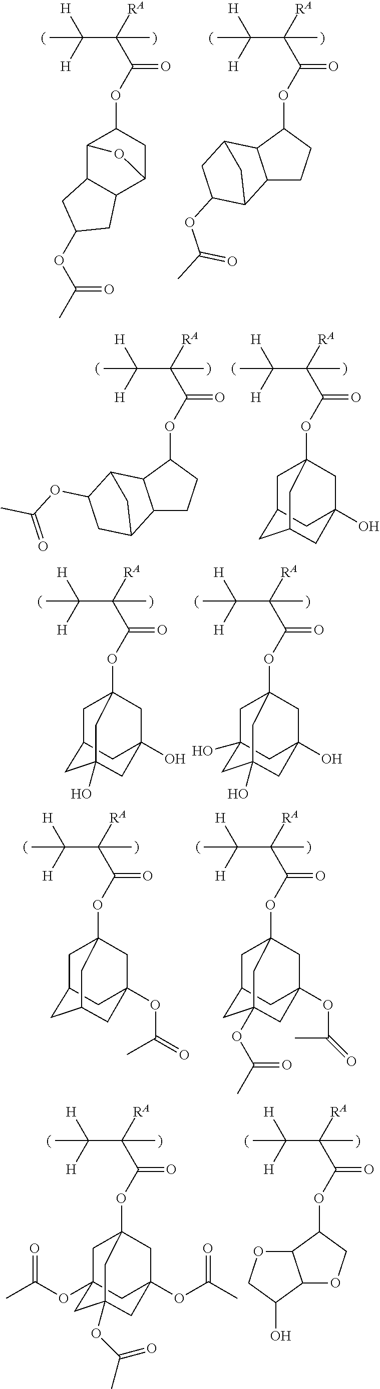

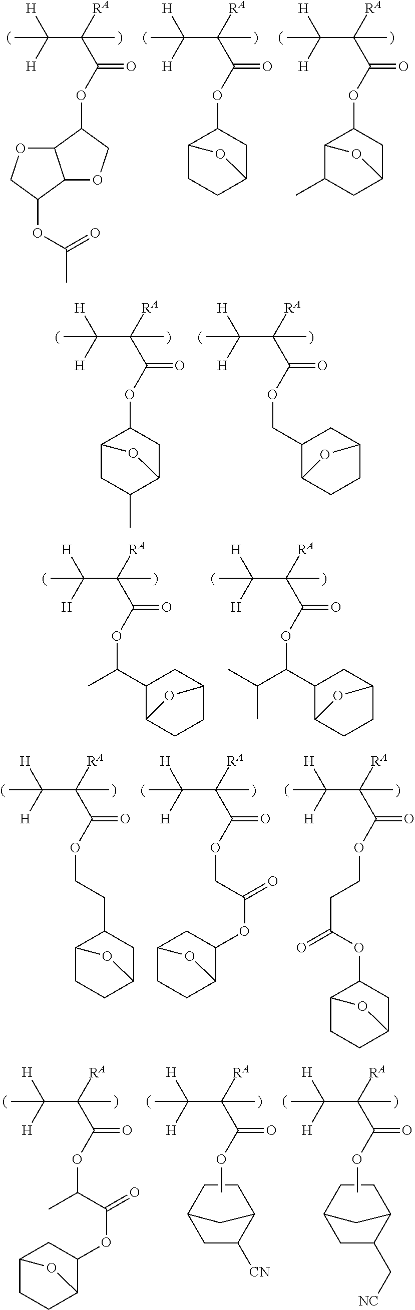

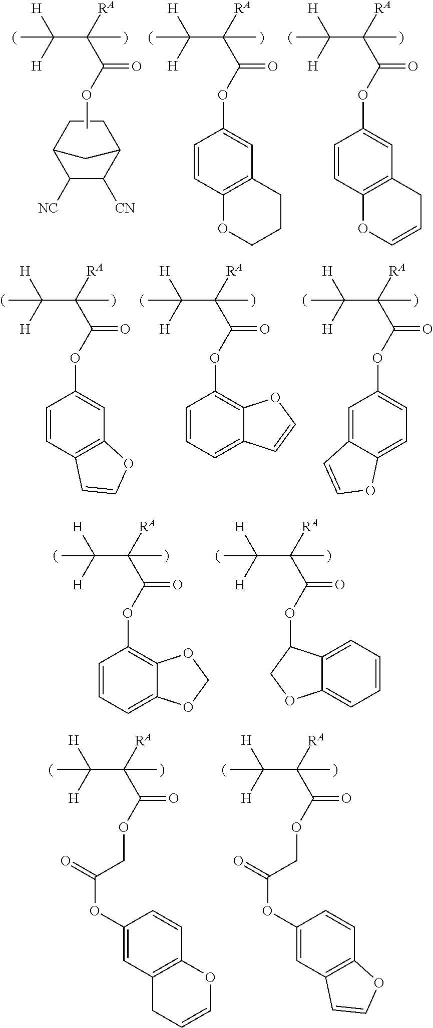

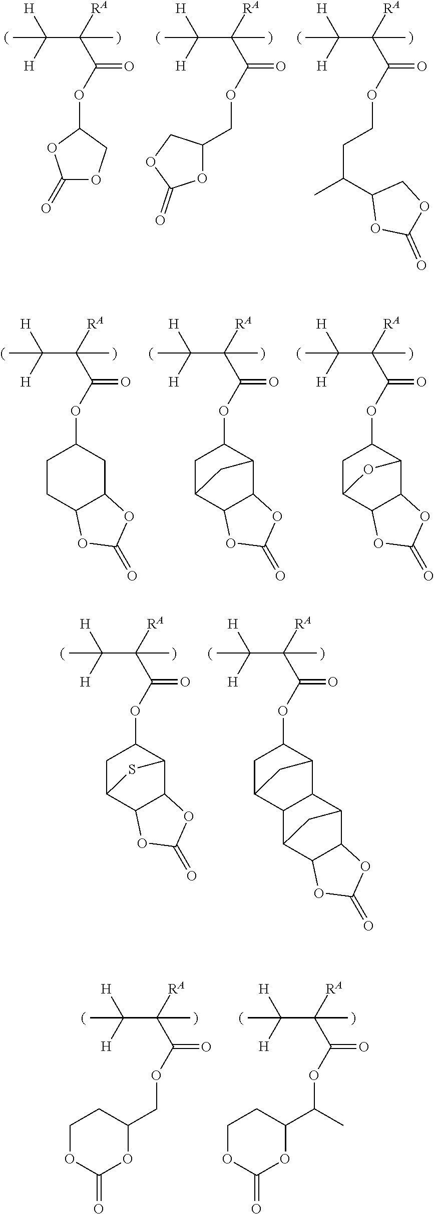

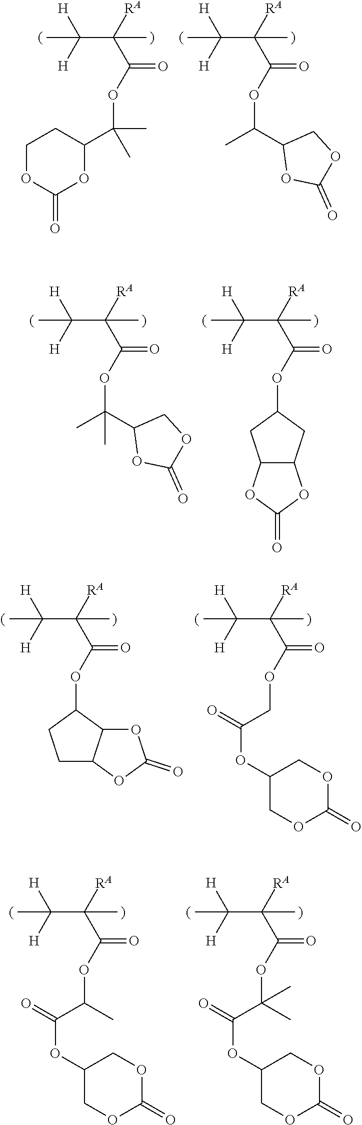

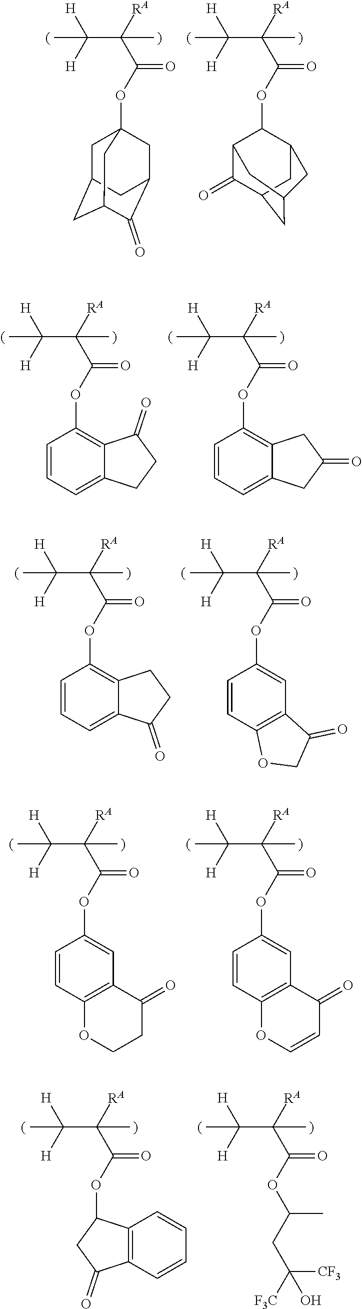

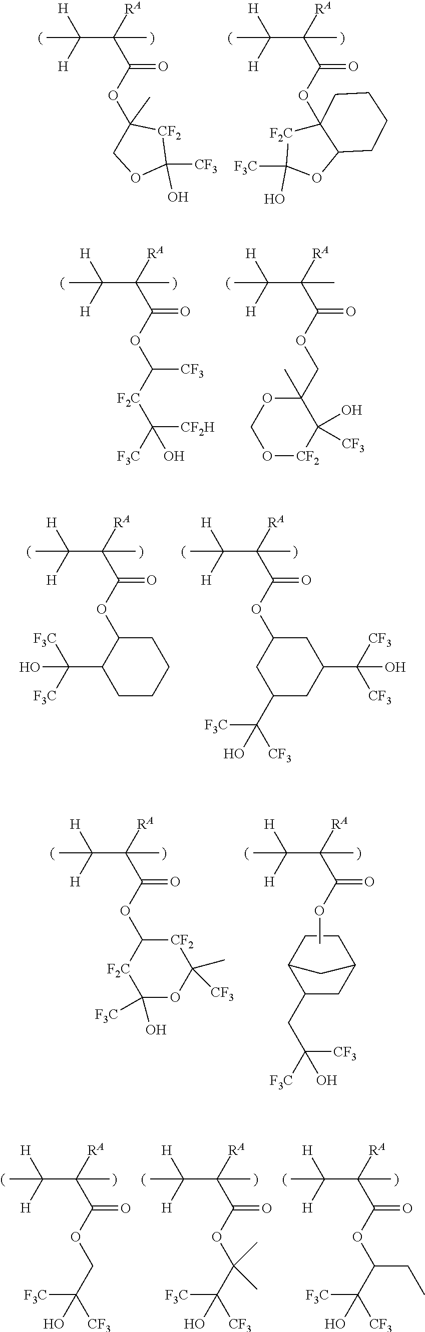

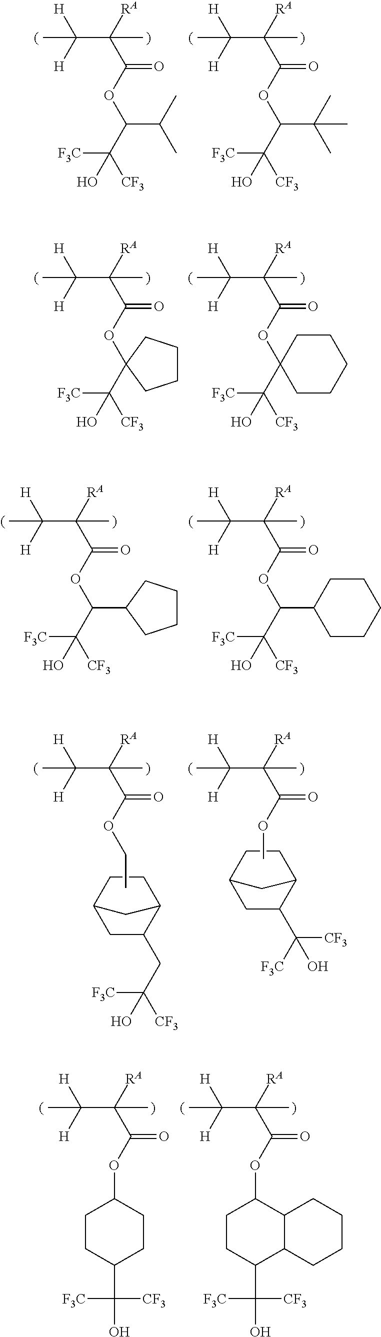

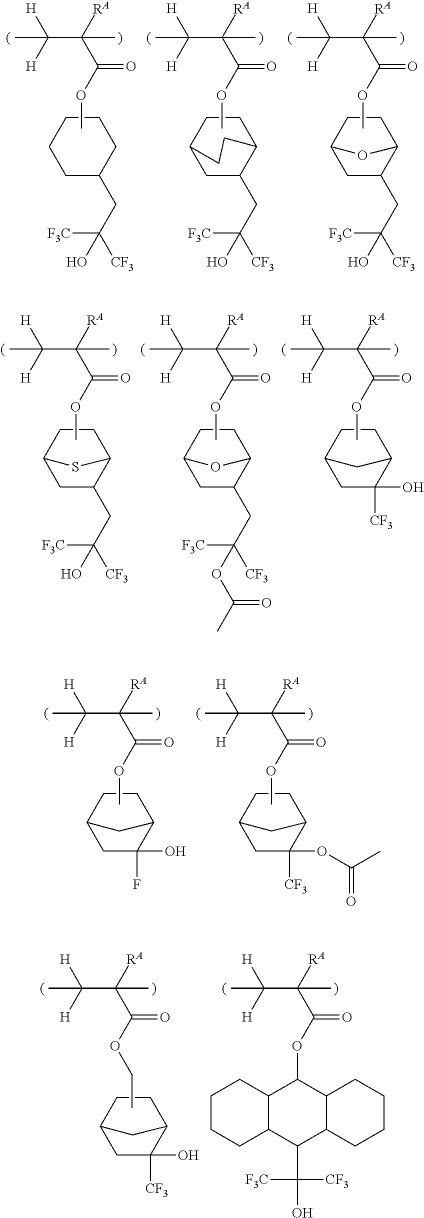

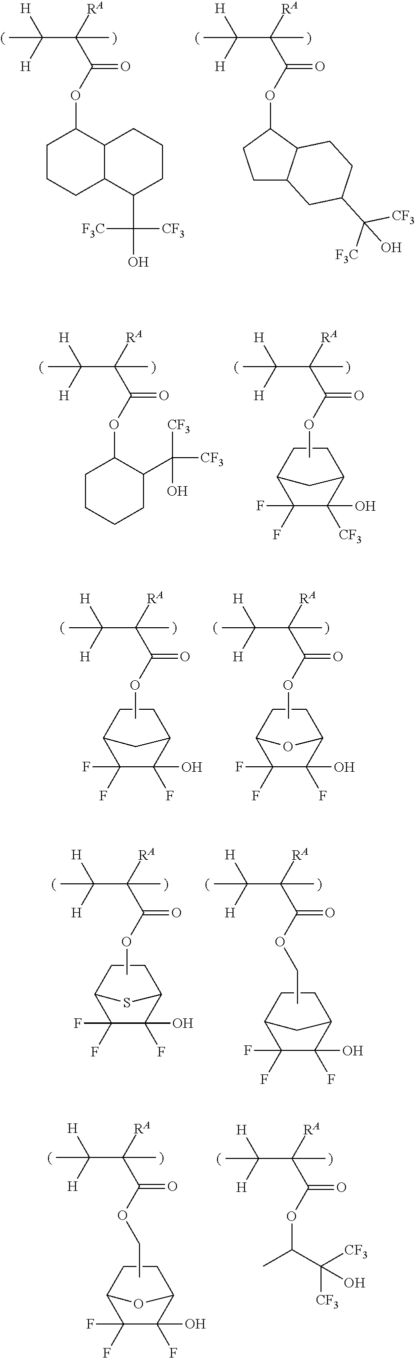

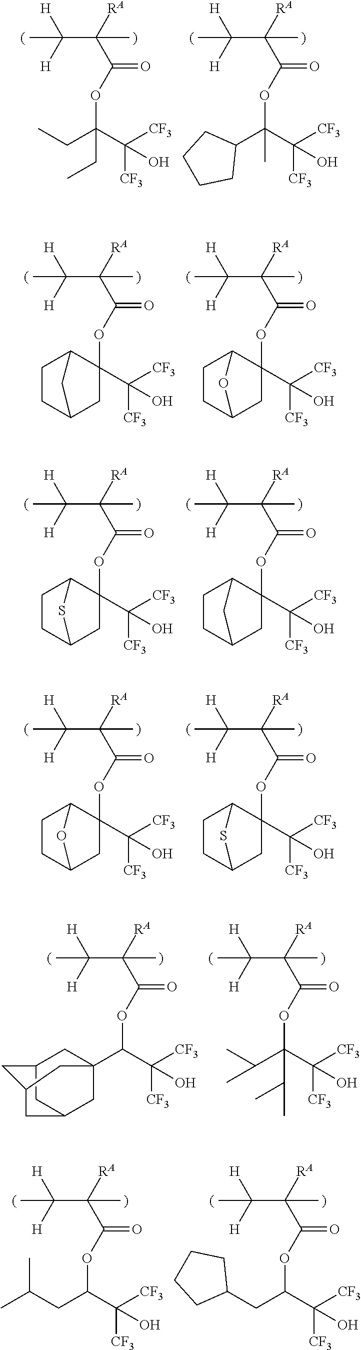

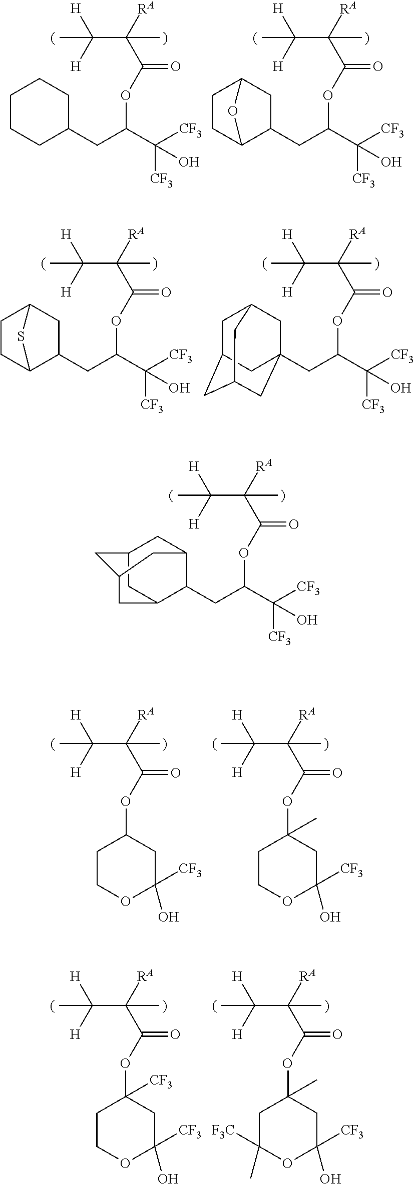

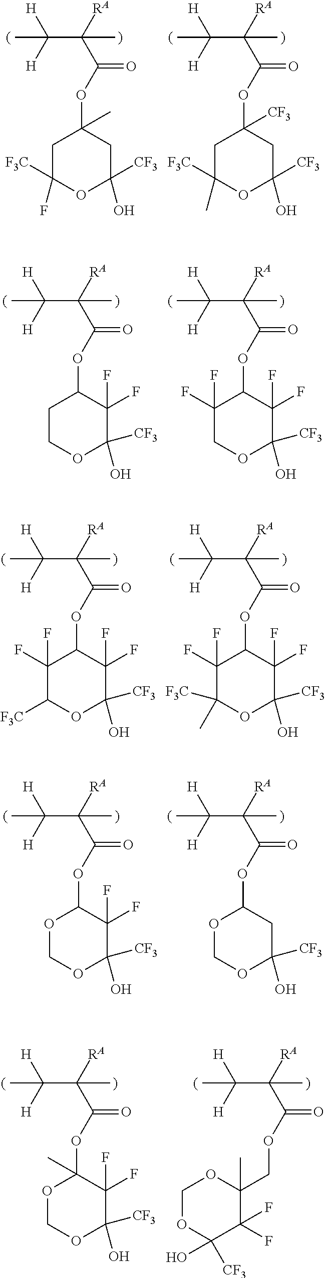

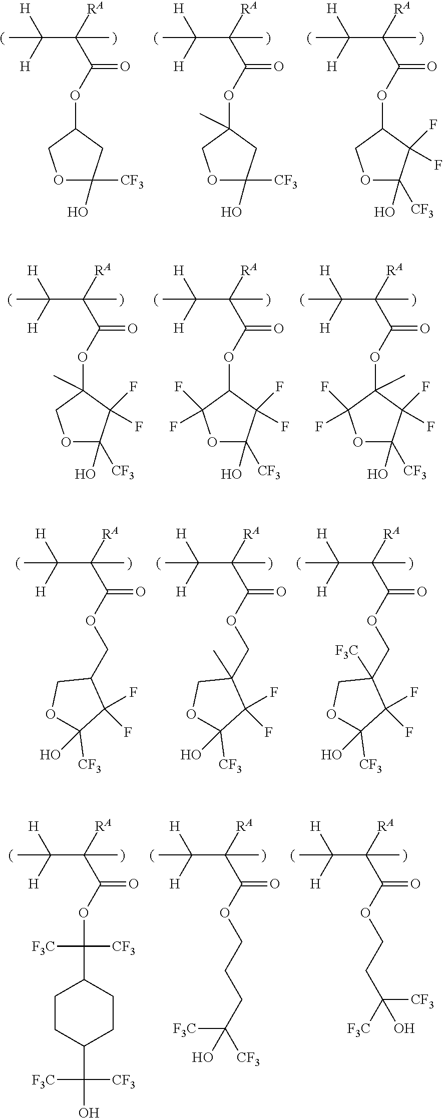

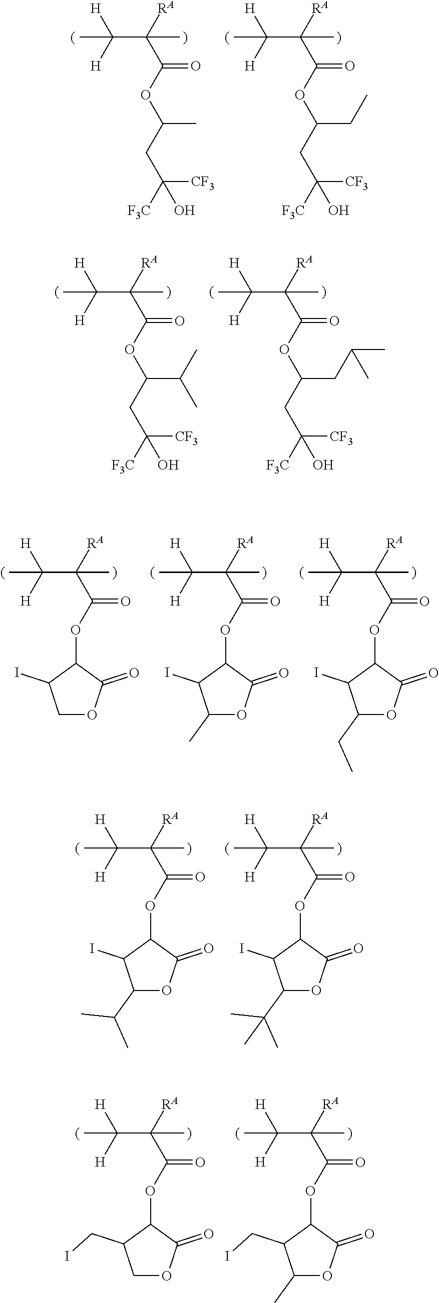

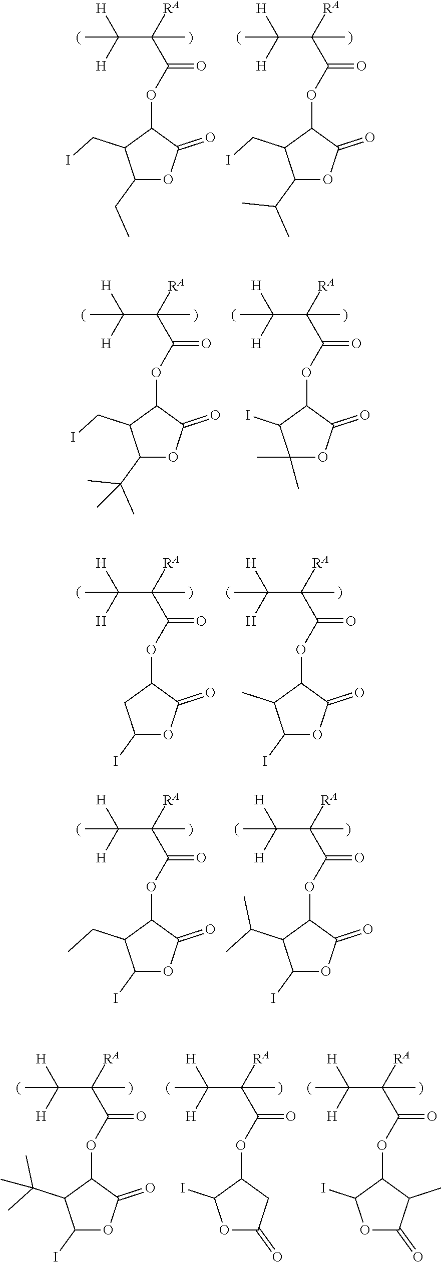

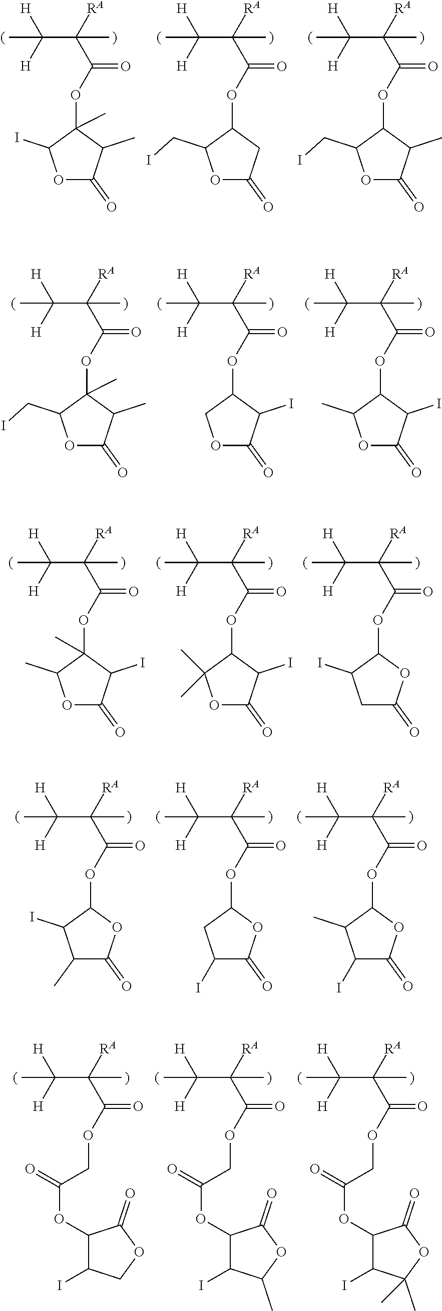

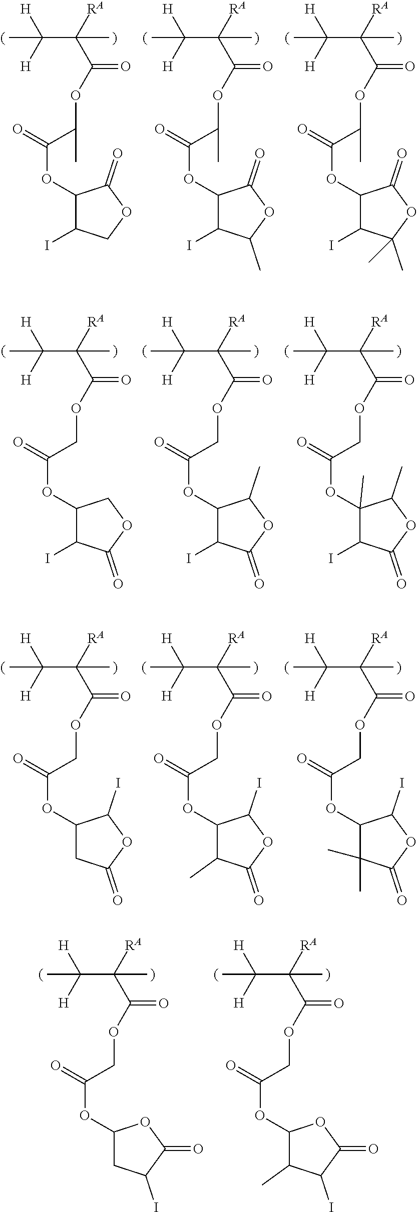

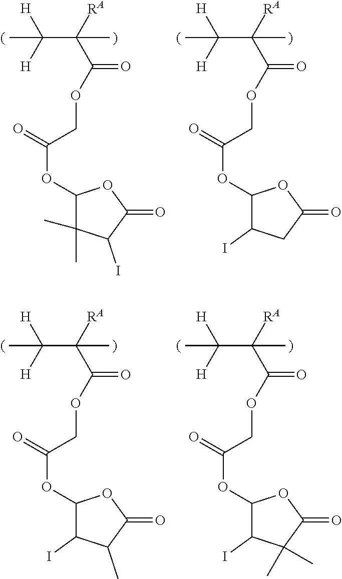

Examples of the repeat unit (b2) are shown below, but not limited thereto. Herein R^{A} is as defined above.

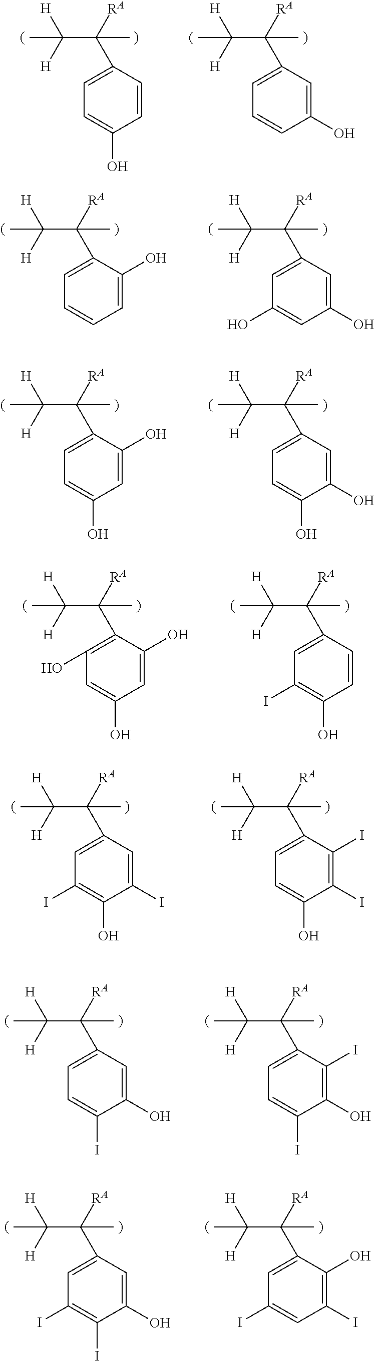

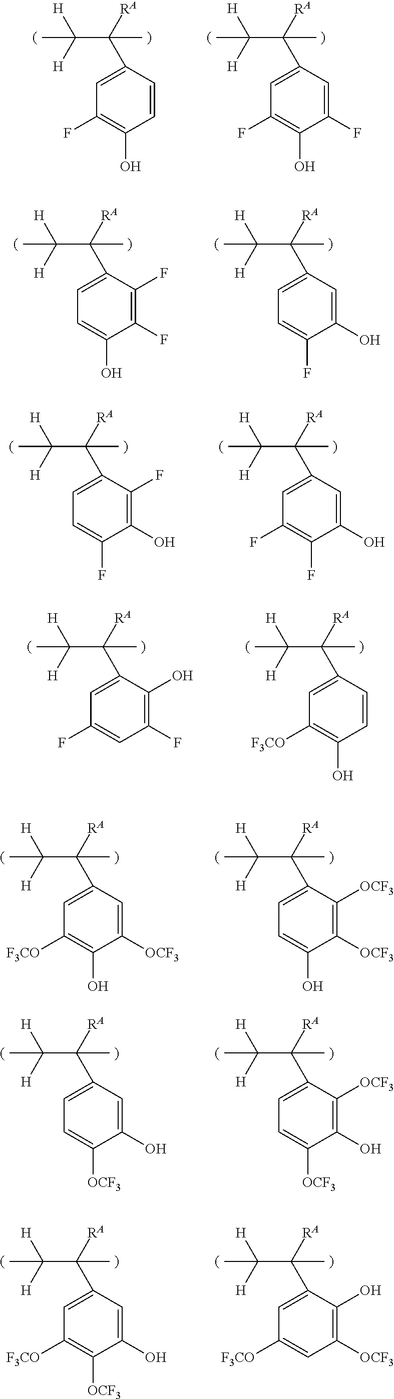

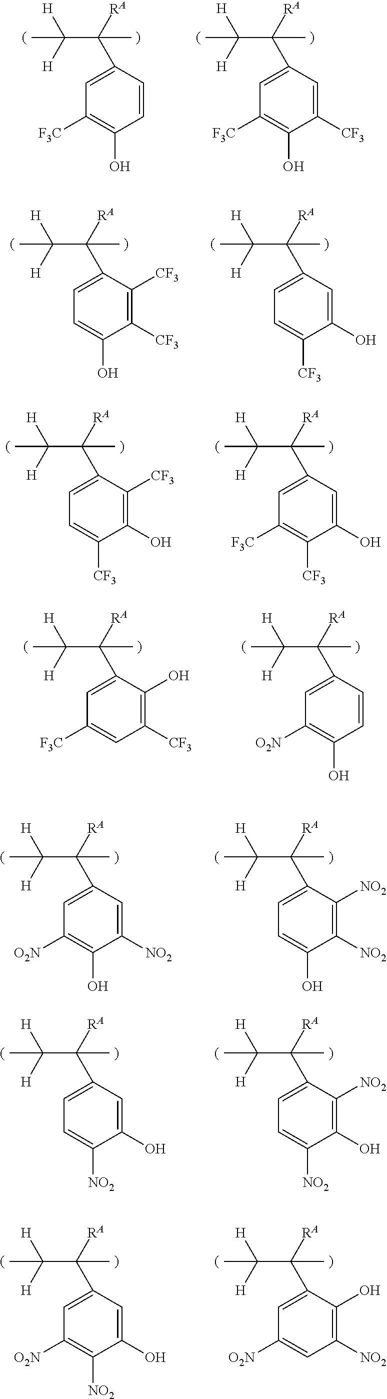

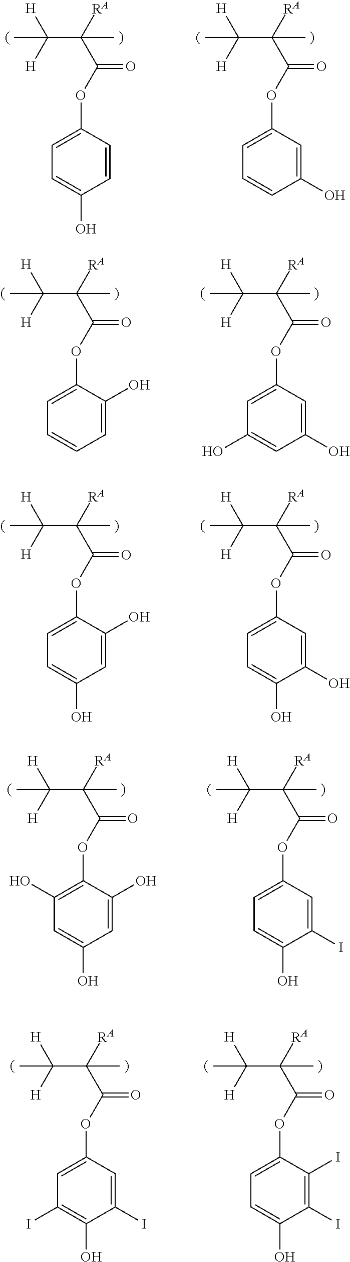

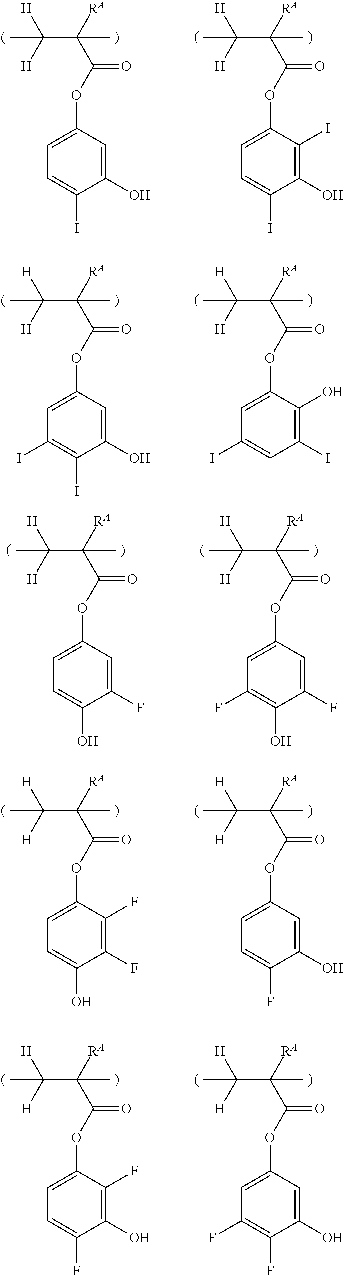

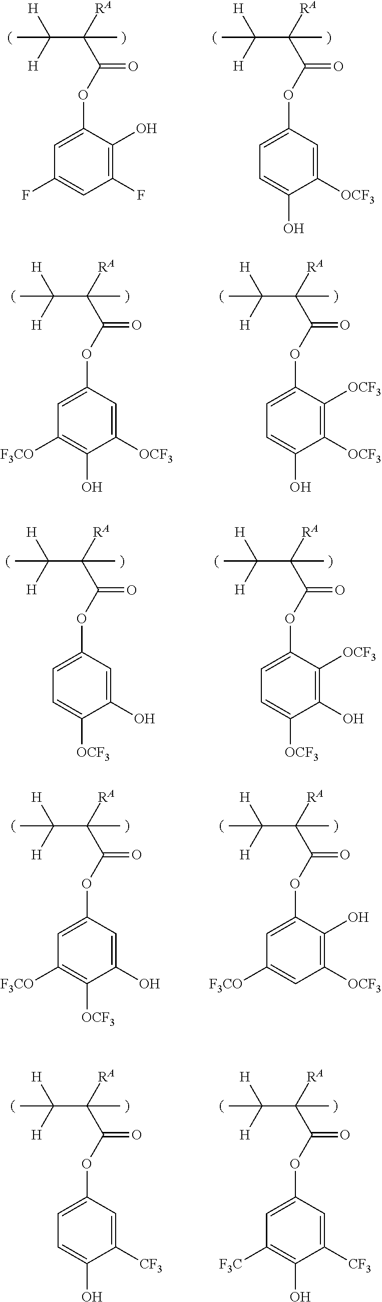

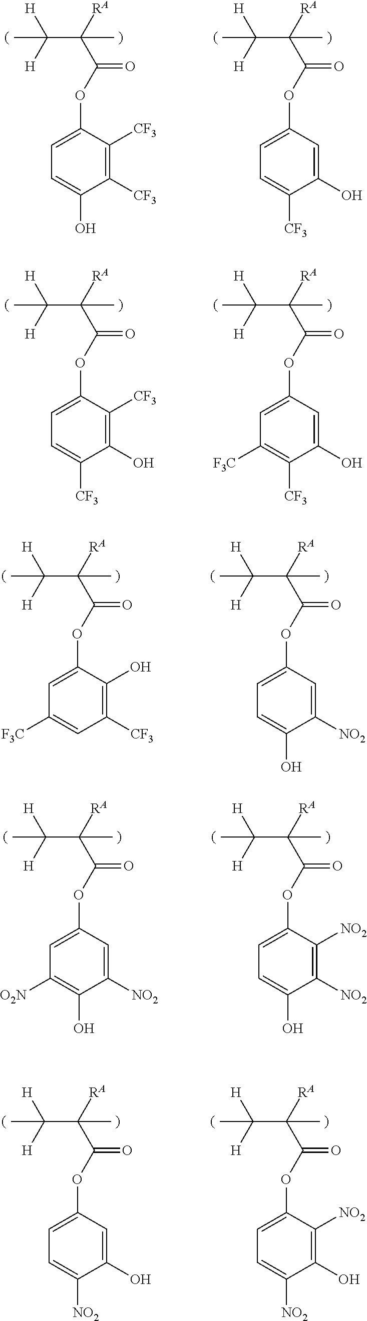

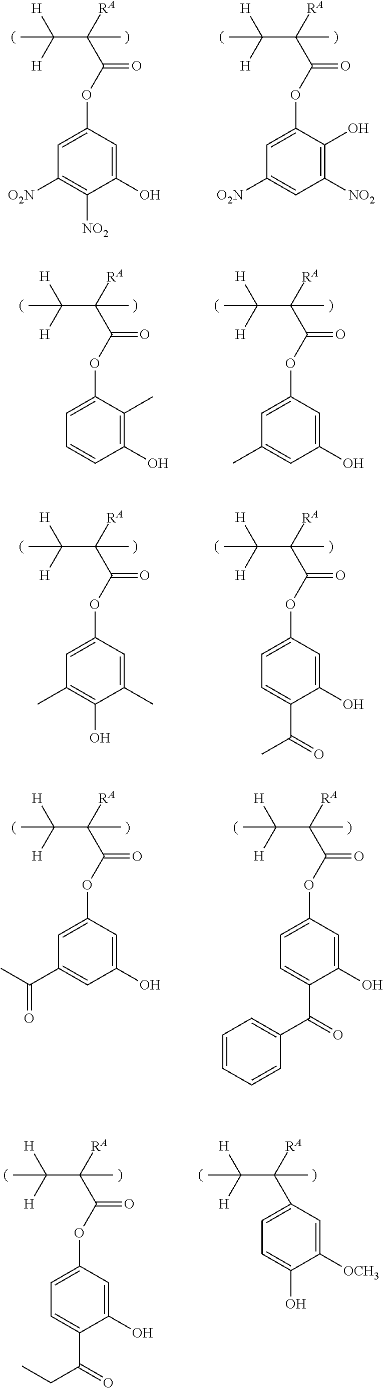

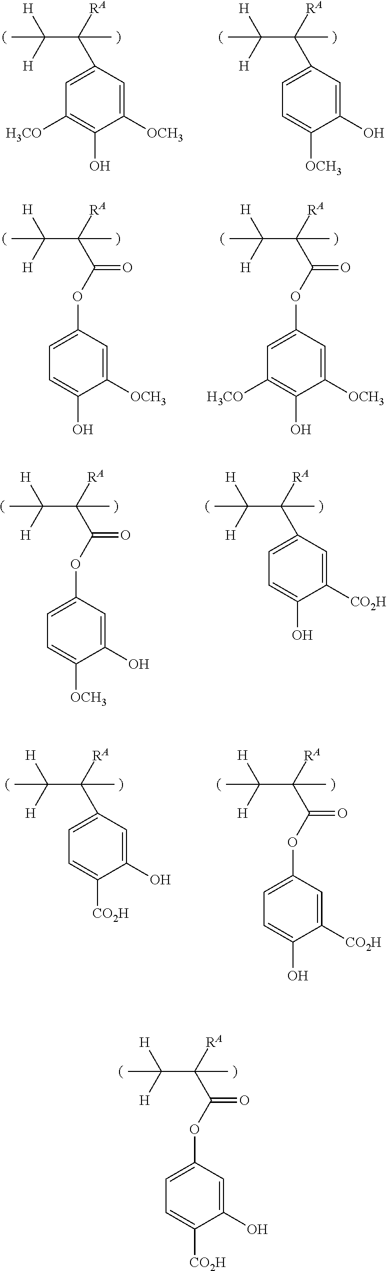

Of the repeat units (b1) and (b2), those units having a lactone ring as the polar group are preferred in the ArF lithography and those units having a phenolic site are preferred in the KrF, EB and EUV lithography.

The polymer may further comprise repeat units (c) of a structure having a hydroxy group protected with an acid labile group. The repeat unit (c) is not particularly limited as long as the unit includes one or more structures having a hydroxy group protected with a protective group such that the protective group is decomposed to generate a hydroxy group under the action of acid. Repeat units having the formula (c1) are preferred.

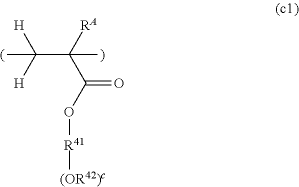

In formula (cl), R^{A} is hydrogen, fluorine, methyl or trifluoromethyl. R⁴¹ is a C₁-C₃₀ (c+1)-valent hydrocarbon group which may contain a heteroatom. R⁴² is an acid labile group, and c is 1, 2, 3 or 4.

In formula (c1), the acid labile group R⁴² is deprotected under the action of acid so that a hydroxy group is generated. Although the structure of R⁴² is not particularly limited, preferred are an acetal structure, ketal structure, hydrocarbyloxycarbonyl group, hydrocarbyloxymethyl group having formula (c2):

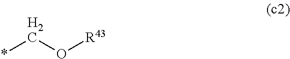

wherein R⁴³ is a C₁-C₁₅ hydrocarbyl group. The hydrocarbyloxymethyl group having formula (c2) is more preferred.

Illustrative examples of the acid labile group R⁴², the hydrocarbyloxymethyl group having formula (c2), and the repeat units (c) are as exemplified for the repeat units (c) in JP-A 2020-111564 (US20200223796).

In addition to the foregoing units, the polymer may further comprise repeat units (d) derived from indene, benzofuran, benzothiophene, acenaphthylene, chromone, coumarin, and norbornadiene, or derivatives thereof. Examples of the monomer from which repeat units (d) are derived are shown below, but not limited thereto.

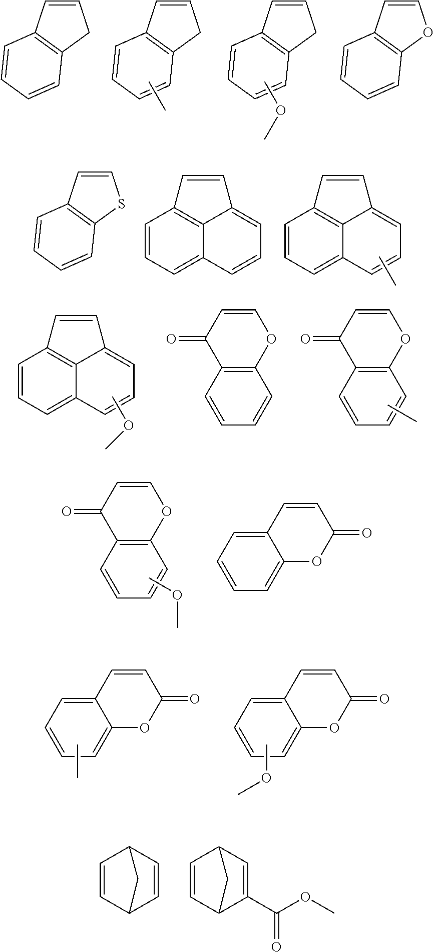

Furthermore, the polymer may comprise repeat units (e) derived from indane, vinylpyridine, vinylcarbazole, or derivatives thereof.

In the polymer, a fraction of units (A), (a1), (a2), (a3), (b1), (b2), (c), (d), and (e) is: preferably 0<A≤0.4, 0≤a1≤0.8, 0≤a2≤0.8, 0≤a3≤0.6, 0<a1+a2+a3≤0.8, 0≤b1≤0.6, 0≤b2≤0.6, 0≤c≤0.5, 0≤d≤0.3, and 0≤e≤0.3; more preferably 0<A≤0.3, 0≤a1≤0.7, 0≤a2≤0.7, 0≤a3<0.5, 0<a1+a2+a3≤0.7, 0≤b1≤0.5, 0≤b2≤0.5, 0≤c≤0.3, 0≤d≤0.3, and 0≤e≤0.3, with the proviso: A+a1+a2+a3+b1+b2+c+d+e≤1.0.

The polymer should preferably have a weight average molecular weight (Mw) in the range of 1,000 to 500,000, and more preferably 3,000 to 100,000. A Mw in the range ensures satisfactory etch resistance and eliminates the risk of resolution being lowered due to a failure to acquire a difference in dissolution rate before and after exposure. It is noted that Mw is as measured by GPC versus polystyrene standards using tetrahydrofuran (THF) or N,N-dimethylformamide (DMF) solvent.

Since the influence of dispersity (Mw/Mn) becomes stronger as the pattern rule becomes finer, the polymer should preferably have a narrow dispersity (Mw/Mn) of 1.0 to 2.0 in order to provide a resist composition suitable for micropatterning to a small feature size. A Mw/Mn in the range indicates smaller amounts of lower and higher molecular weight fractions and eliminates the risk of leaving foreign particles on the pattern or degrading the pattern profile after exposure and development.

The polymer may be synthesized by any desired methods, for example, by dissolving one or more monomers selected from the monomers corresponding to the foregoing repeat units in an organic solvent, adding a radical polymerization initiator thereto, and heating for polymerization. Examples of the organic solvent which can be used for polymerization include toluene, benzene, tetrahydrofuran (THF), diethyl ether, dioxane, cyclohexane, cyclopentane, methyl ethyl ketone (MEK), PGMEA, and GBL. Examples of the polymerization initiator used herein include 2,2′-azobisisobutyronitrile (AIBN), 2,2′-azobis(2,4-dimethylvaleronitrile), dimethyl 2,2-azobis(2-methylpropionate), 1,1′-azobis(1-acetoxy-1-phenylethane), benzoyl peroxide, and lauroyl peroxide. The amount of the initiator added is preferably 0.01 to 25 mol % based on the total of monomers. The reaction temperature is preferably 50 to 150° C., more preferably 60 to 100° C. The reaction time is preferably 2 to 24 hours, a time of 2 to 12 hours being more preferred in view of production efficiency.

The polymerization initiator may be added to the monomer solution, which is fed to the reactor. Alternatively, a solution of the polymerization initiator is prepared separately from the monomer solution, and the monomer and initiator solutions are independently fed to the reactor. Since there is a possibility that the initiator generates a radical in the standby time, by which polymerization reaction takes place to form a ultrahigh molecular weight compound, it is preferred from the standpoint of quality control that the monomer solution and the initiator solution be independently prepared and added dropwise. The acid labile group that has been incorporated in the monomer may be kept as such, or the polymerization may be followed by protection or partial protection. Any of well-known chain transfer agents such as dodecylmercaptan and 2-mercaptoethanol may be used for the purpose of adjusting molecular weight. An appropriate amount of the chain transfer agent is 0.01 to 20 mol % based on the total of monomers to be polymerized.

Where a monomer having a hydroxy group is copolymerized, the hydroxy group may be replaced by an acetal group susceptible to deprotection with acid, typically ethoxyethoxy, prior to polymerization, and the polymerization be followed by deprotection with weak acid and water. Alternatively, the hydroxy group may be replaced by an acetyl, formyl, pivaloyl or similar group prior to polymerization, and the polymerization be followed by alkaline hydrolysis.

When hydroxystyrene or hydroxyvinylnaphthalene is copolymerized, an alternative method is possible. Specifically, acetoxystyrene or acetoxyvinylnaphthalene is used instead of hydroxystyrene or hydroxyvinylnaphthalene, and after polymerization, the acetoxy group is deprotected by alkaline hydrolysis, for thereby converting the polymer product to hydroxystyrene or hydroxyvinylnaphthalene. For alkaline hydrolysis, a base such as aqueous ammonia or triethylamine may be used. Preferably the reaction temperature is −20° C. to 100° C., more preferably 0° C. to 60° C., and the reaction time is 0.2 to 100 hours, more preferably 0.5 to 20 hours.

The amounts of monomers in the monomer solution may be determined appropriate so as to provide the preferred fractions of repeat units as mentioned above.

It is described how to use the polymer obtained by the above preparation method. The reaction solution resulting from polymerization reaction may be used as the final product. Alternatively, the polymer may be recovered in powder form through a purifying step such as re-precipitation step of adding the reaction solution to a poor solvent and letting the polymer precipitate as powder, after which the polymer powder is used as the final product. It is preferred from the standpoints of operation efficiency and consistent quality to handle a polymer solution which is obtained by dissolving the powder polymer resulting from the purifying step in a solvent, as the final product.

The solvents which can be used herein are described in JP-A 2008-111103, paragraphs [0144]-[0145] (U.S. Pat. No. 7,537,880). Exemplary solvents include ketones such as cyclohexanone and methyl-2-n-pentyl ketone: alcohols such as 3-methoxy butanol, 3-methyl-3-methoxy butanol, 1-methoxy-2-propanol, 1-ethoxy-2-propanol, and diacetone alcohol (DAA): ethers such as propylene glycol monomethyl ether (PGME), ethylene glycol monomethyl ether, propylene glycol monoethyl ether, ethylene glycol monoethyl ether, propylene glycol dimethyl ether, and diethylene glycol dimethyl ether: esters such as PGMEA, propylene glycol monoethyl ether acetate, ethyl lactate, ethyl pyruvate, butyl acetate, methyl 3-methoxypropionate, ethyl 3-ethoxypropionate, tert-butyl acetate, tert-butyl propionate, and propylene glycol mono-tert-butyl ether acetate: lactones such as GBL; and high-boiling alcohols such as diethylene glycol, propylene glycol, glycerol, 1,4-butanediol, and 1,3-butanediol, which may be used alone or in admixture.

The polymer solution preferably has a polymer concentration of 0.01 to 30% by weight, more preferably 0.1 to 20% by weight.

Prior to use, the reaction solution or polymer solution is preferably filtered through a filter. Filtration is effective for consistent quality because foreign particles and gel which can cause defects are removed.

Suitable materials of which the filter is made include fluorocarbon, cellulose, nylon, polyester, and hydrocarbon base materials. Preferred for the filtration of a resist composition are filters made of fluorocarbons commonly known as Teflon®, hydrocarbons such as polyethylene and polypropylene, and nylon. While the pore size of the filter may be selected appropriate to comply with the desired cleanness, the filter preferably has a pore size of up to 100 nm, more preferably up to 20 nm. A single filter may be used or a plurality of filters may be used in combination. Although the filtering method may be single pass of the solution, preferably the filtering step is repeated by flowing the solution in a circulating manner. In the polymer preparation process, the filtering step may be carried out any times, in any order and in any stage. The reaction solution as polymerized or the polymer solution may be filtered, preferably both are filtered.

### [Chemically Amplified Resist Composition]

A further embodiment of the invention is a chemically amplified resist composition comprising (A) a base polymer containing the polymer defined above.

### (A) Base Polymer

The polymer defined above may be used alone or as a mixture of two or more polymers which are different in compositional ratio, Mw and/or Mw/Mn. In addition to the polymer, the base polymer (A) may contain a hydrogenated product of ring-opening metathesis polymerization (ROMP) polymer, which is described in JP-A 2003-066612.

### (B) Organic Solvent

The resist composition may comprise (B) an organic solvent. The organic solvent used herein is not particularly limited as long as the foregoing and other components are soluble therein. Suitable solvents include ketones such as cyclopentanone, cyclohexanone, and methyl-2-n-pentyl ketone; alcohols such as 3-methoxybutanol, 3-methyl-3-methoxybutanol, 1-methoxy-2-propanol, and 1-ethoxy-2-propanol: keto-alcohols such as diacetone alcohol (DAA); ethers such as propylene glycol monomethyl ether (PGME), ethylene glycol monomethyl ether, propylene glycol monoethyl ether, ethylene glycol monoethyl ether, propylene glycol dimethyl ether, and diethylene glycol dimethyl ether; esters such as propylene glycol monomethyl ether acetate (PGMEA), propylene glycol monoethyl ether acetate, ethyl lactate (EL), ethyl pyruvate, butyl acetate, methyl 3-methoxypropionate, ethyl 3-ethoxypropionate, tert-butyl acetate, tert-butyl propionate, and propylene glycol mono-tert-butyl ether acetate; and lactones such as γ-butyrolactone (GBL), and mixtures thereof.

Of the foregoing organic solvents, it is recommended to use 1-ethoxy-2-propanol, PGMEA, cyclohexanone, GBL, EL, DAA, and mixtures thereof because the base polymer (A) is most soluble therein.

The organic solvent (C) is preferably added in an amount of 200 to 7,000 parts by weight, and more preferably 400 to 5,000 parts by weight per 80 parts by weight of the base polymer (A). The organic solvent may be used alone or in admixture.

### (C) Quencher

The resist composition may further comprise (C) a quencher. As used herein, the “quencher” refers to a compound capable of trapping the acid generated by the PAG to prevent the acid from diffusing into the unexposed region of resist film, for forming the desired pattern.

Preferred examples of the quencher include onium salts having the formulae (1) and (2).

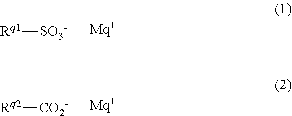

In formula (1), Ral is hydrogen or a C₁-C₄₀ hydrocarbyl group which may contain a heteroatom, exclusive of the group wherein hydrogen bonded to the carbon atom at α-position relative to the sulfo group is substituted by fluorine or fluoroalkyl. In formula (2), R⁹² is hydrogen or a C₁-C₄₀ hydrocarbyl group which may contain a heteroatom

Examples of the C₁-C₄₀ hydrocarbyl group R⁹¹ include C₁-C₄₀ alkyl groups such as methyl, ethyl, n-propyl, isopropyl, n-butyl, isobutyl, sec-butyl, tert-butyl, n-pentyl, tert-pentyl, n-hexyl, n-octyl, 2-ethylhexyl, n-nonyl, and n-decyl; C₃-C₄₀ cyclic saturated hydrocarbyl groups such as cyclopentyl, cyclohexyl, cyclopentylmethyl, cyclopentylethyl, cyclopentylbutyl, cyclohexylmethyl, cyclohexylethyl, cyclohexylbutyl, norbornyl, tricyclo[5.2.1.0^{2,6}]decyl, and adamantyl; C₆-C₄₀ aryl groups such as phenyl, naphthyl and anthracenyl. In the hydrocarbyl group, some or all hydrogen may be substituted by a moiety containing a heteroatom such as oxygen, sulfur, nitrogen or halogen, and some constituent —CH₂— may be replaced by a moiety containing a heteroatom such as oxygen, sulfur or nitrogen, so that the group may contain a hydroxy moiety, fluorine, chlorine, bromine, iodine, cyano moiety, carbonyl moiety, ether bond, ester bond, sulfonic ester bond, carbonate bond, lactone ring, sultone ring, carboxylic anhydride (—C(═O)—O—C(═O)—), or haloalkyl moiety.

Examples of the hydrocarbyl group R^{q2} include those exemplified above for Ral and fluorinated saturated hydrocarbyl groups, for example, fluorinated alkyl groups such as trifluoromethyl and trifluoroethyl, and fluorinated aryl groups such as pentafluorophenyl and 4-trifluoromethylphenyl.

Examples of the anion in the onium salt having formula (1) are shown below, but not limited thereto.

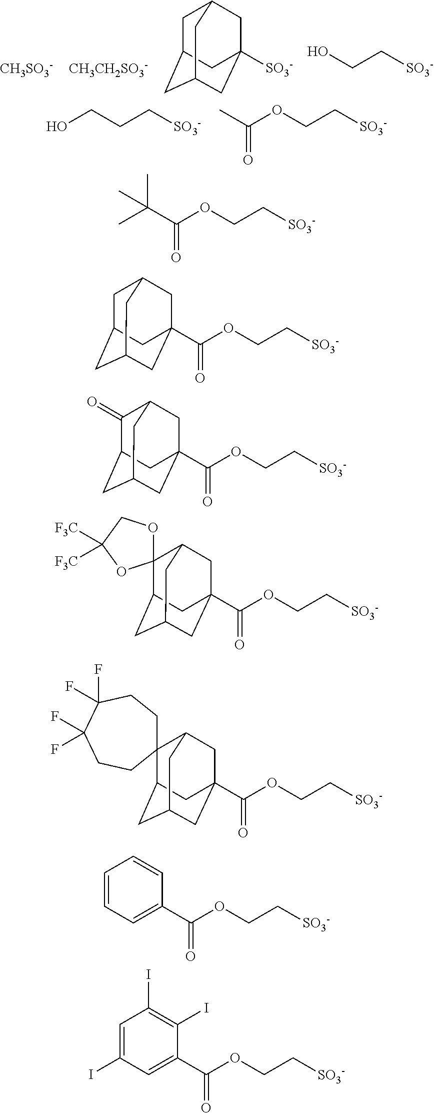

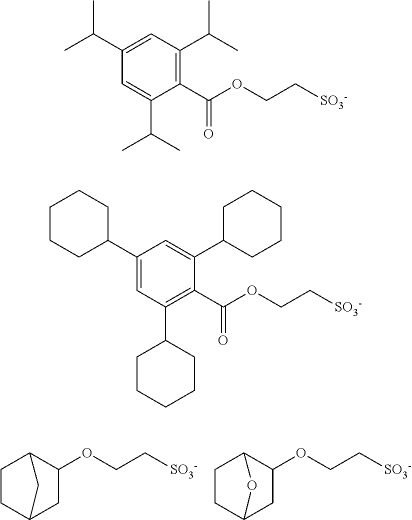

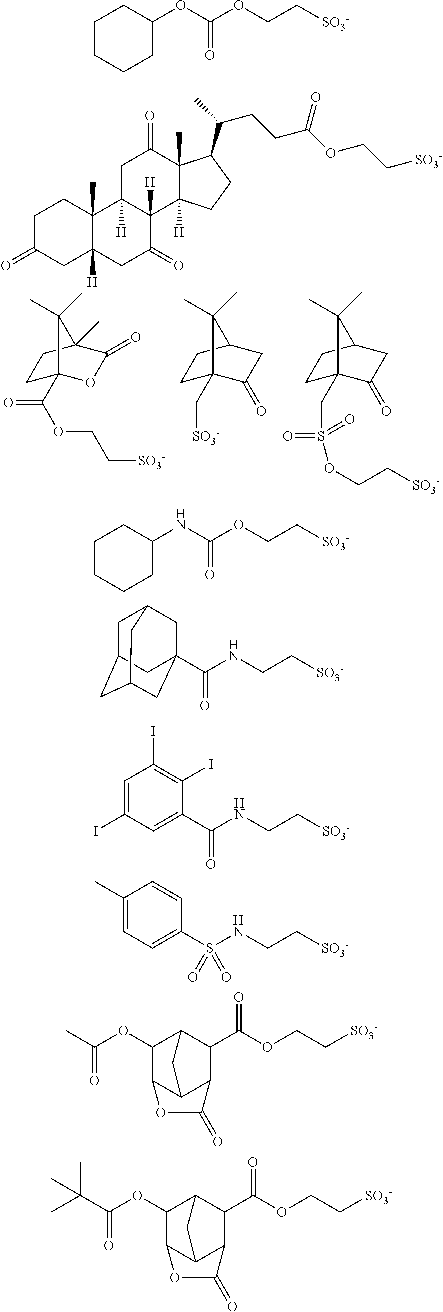

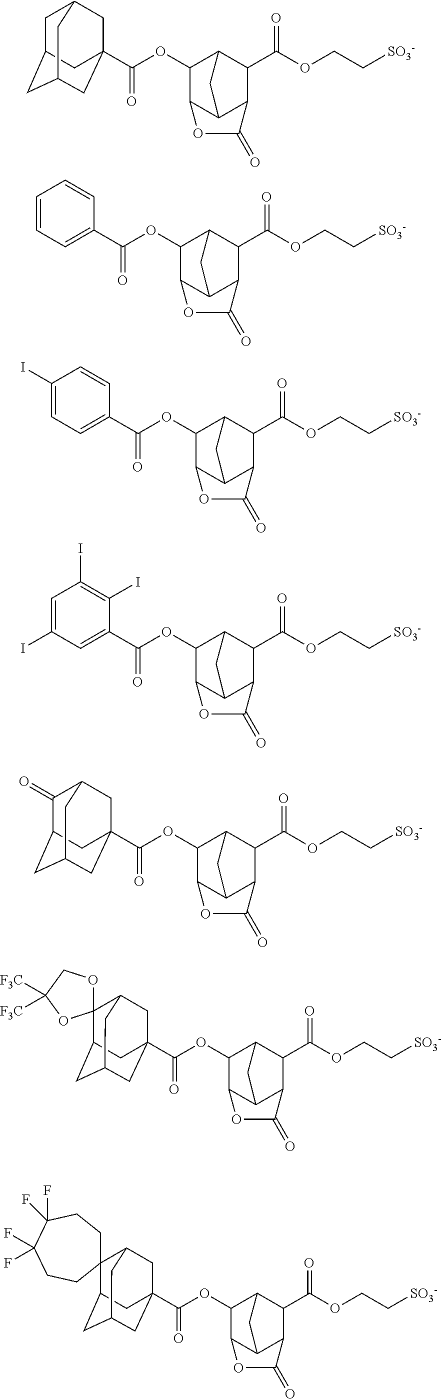

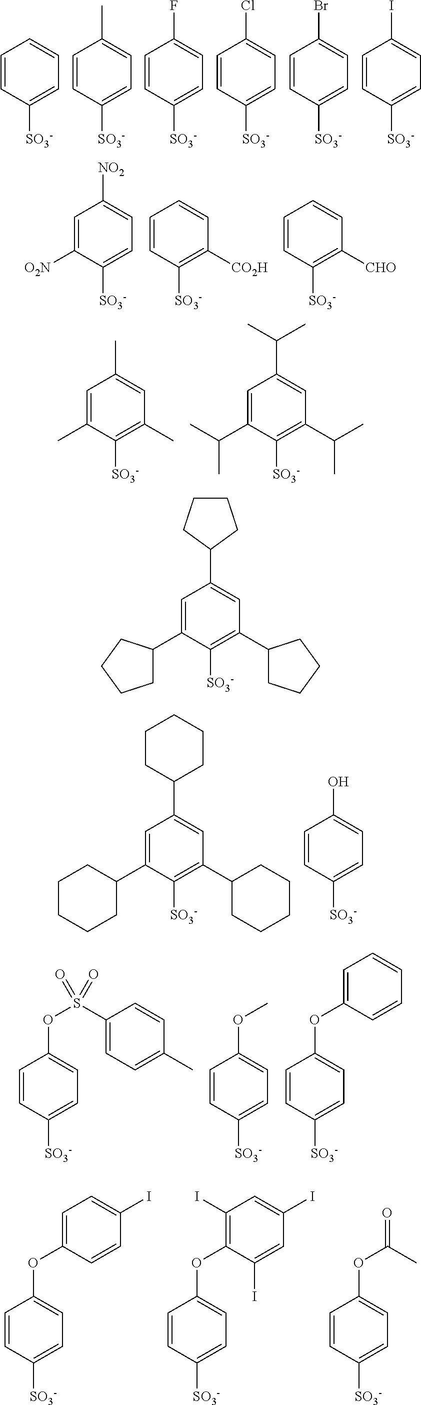

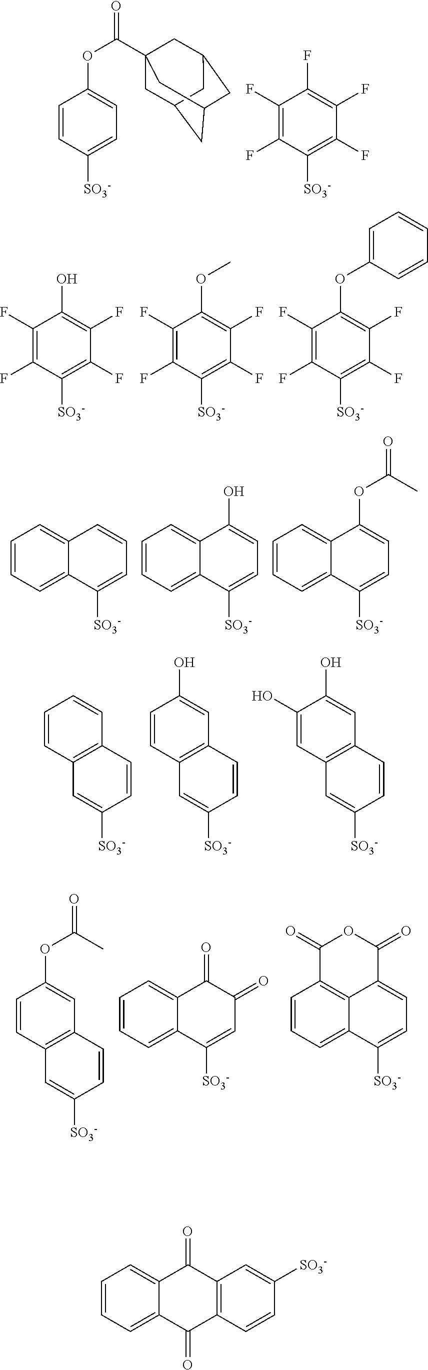

Examples of the anion in the onium salt having formula (2) are shown below, but not limited thereto.

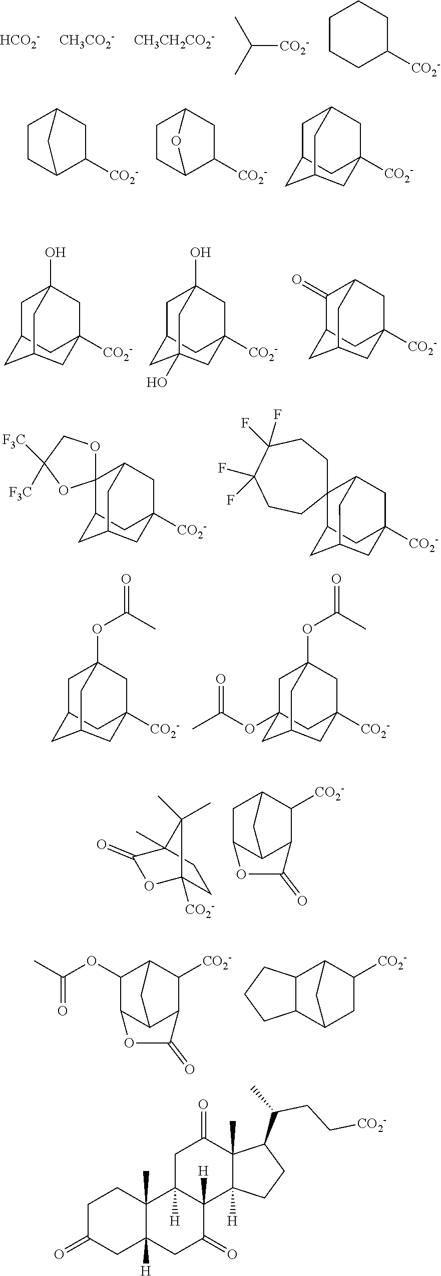

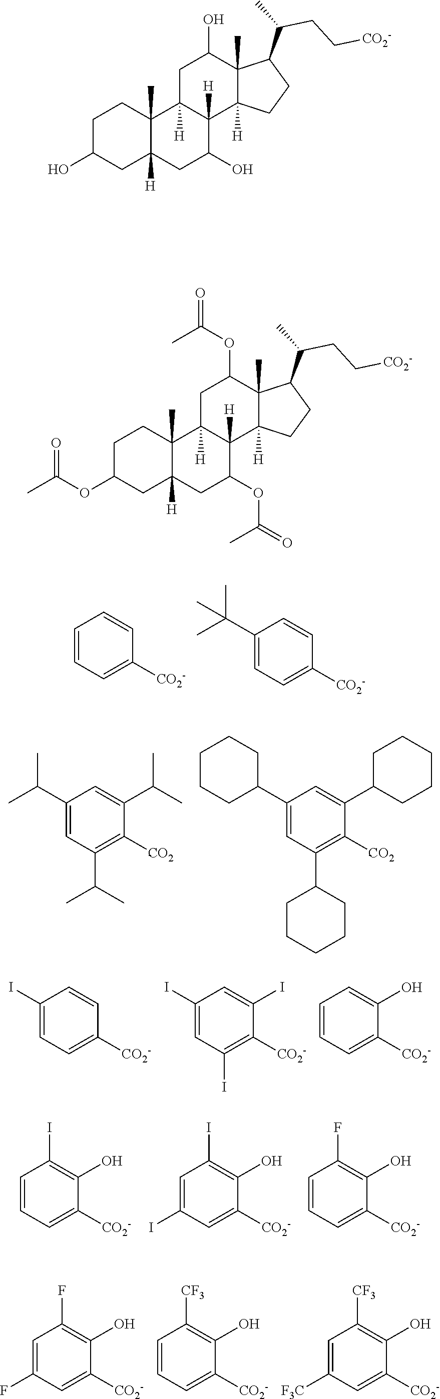

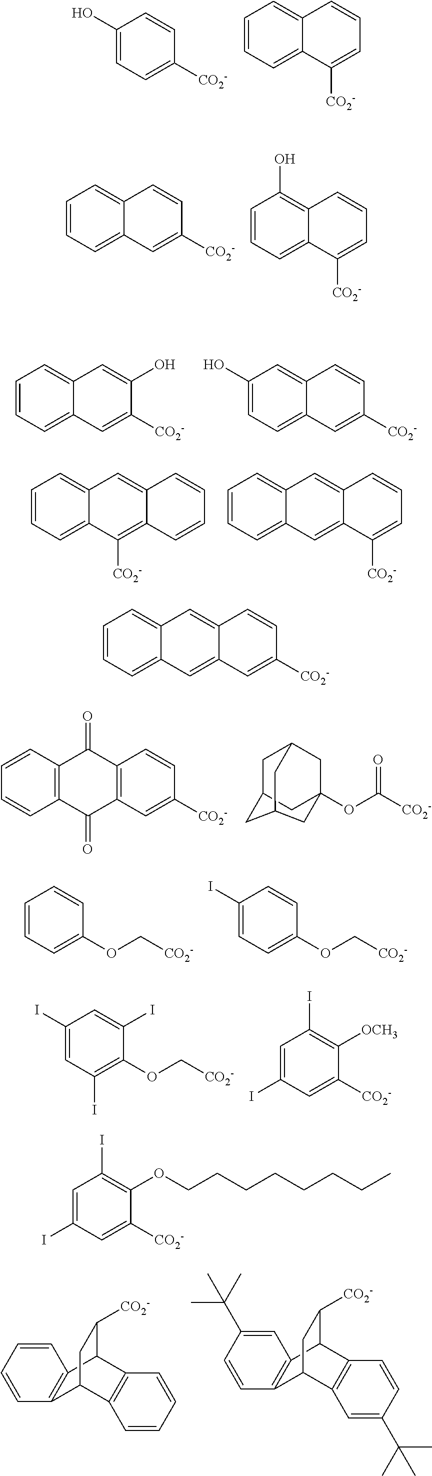

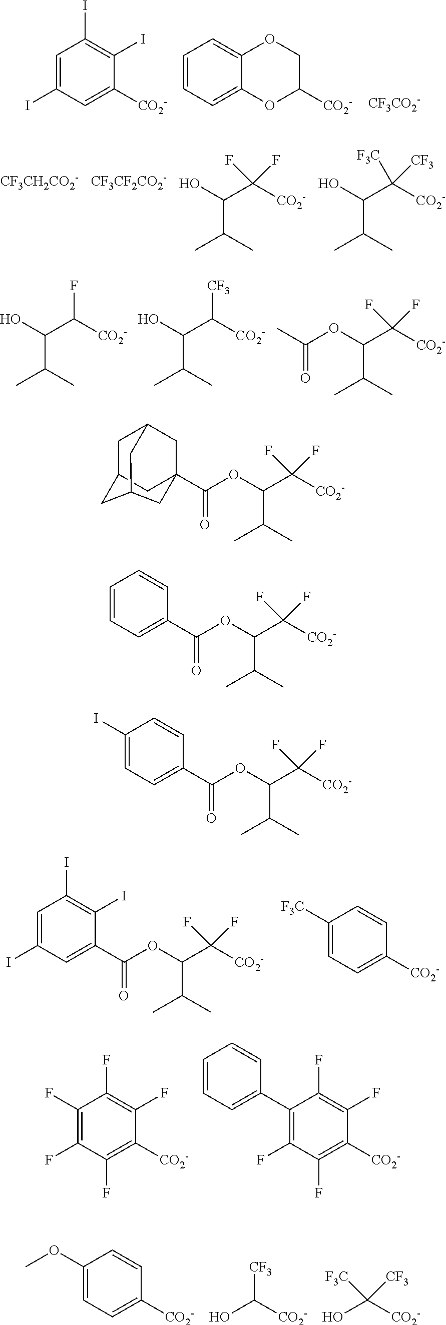

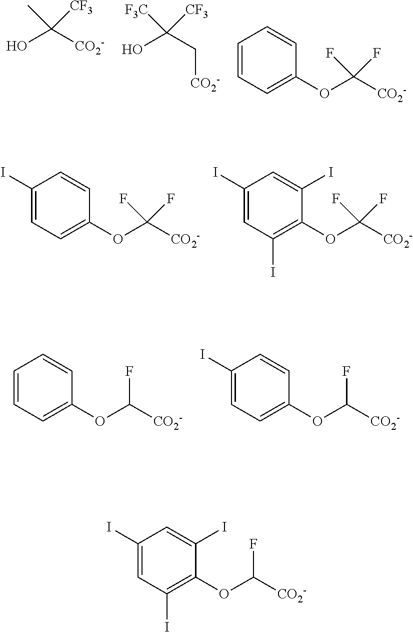

In formulae (1) and (2), Mq⁺ is an onium cation. The onium cation is typically a sulfonium, iodonium or ammonium cation. Examples of the sulfonium cation include those exemplified above as the sulfonium cations having formulae (Z-1) and (Z-3), and those described in JP-A 2024-003744, paragraphs [0102]-[0125], WO 2024/128017, paragraphs [0044]-[0049], and JP 7491173, paragraphs [0035]-[0046].

Examples of the iodonium cation include those described in JP-A 2024-000259, paragraph [0181], but are not limited thereto.

The ammonium cation preferably has the formula (am-1).

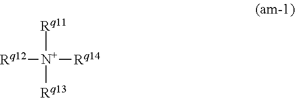

In formula (am-1), R^{q11} to R^{q14} are each independently a C₁-C₄₀ hydrocarbyl group which may contain a heteroatom. R^{q11} and R^{q12} may bond together to form a ring with the nitrogen atom to which they are attached. Examples of the hydrocarbyl group are as exemplified above for the hydrocarbyl group R¹ in formula (A).

Examples of the ammonium cation having formula (am-1) are shown below, but not limited thereto.

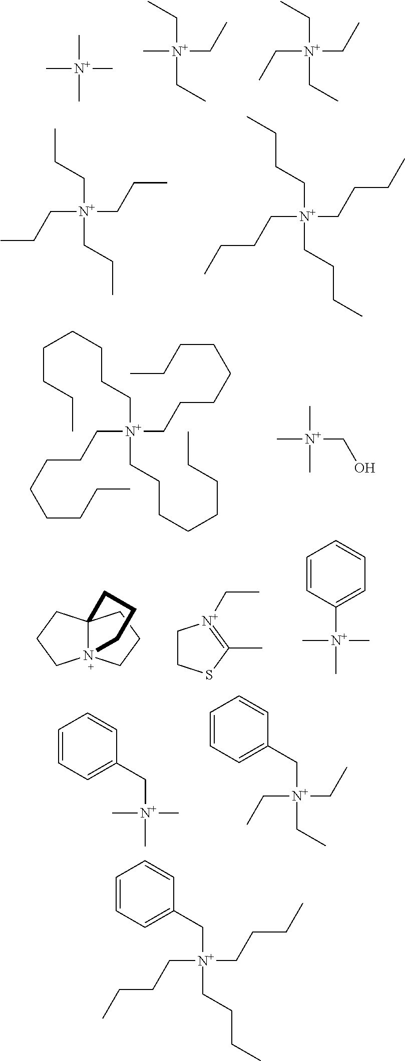

Examples of the onium salt having formula (1) or (2) include arbitrary combinations of anions with cations, both as exemplified above. These onium salts may be readily prepared by ion exchange reaction using any well-known organic chemistry technique. For the ion exchange reaction, reference may be made to JP-A 2007-145797, for example.

The onium salt having formula (1) or (2) functions as a quencher in the chemically amplified resist composition because the counter anion of the onium salt is a conjugated base of a weak acid. As used herein, the weak acid indicates an acidity insufficient to deprotect an acid labile group from an acid labile group-containing unit in the base polymer. The onium salt having formula (1) or (2) functions as a quencher when used in combination with an onium salt type PAG having a conjugated base of a strong acid (typically α-fluorinated sulfonic acid) as the counter anion. In a system using a mixture of an onium salt capable of generating a strong acid (typically α-fluorinated sulfonic acid) and an onium salt capable of generating a weak acid (typically non-fluorinated sulfonic acid or carboxylic acid), if the strong acid generated from the PAG upon exposure to high-energy radiation collides with the unreacted onium salt having a weak acid anion, then a salt exchange occurs whereby the weak acid is released and an onium salt having a strong acid anion is formed. In this course, the strong acid is exchanged into the weak acid having a low catalysis, incurring apparent deactivation of the acid for enabling to control acid diffusion.

Also useful as the quencher (C) are onium salts having sulfonium cation and phenoxide anion sites in a common molecule as described in JP 6848776, onium salts having sulfonium cation and carboxylate anion sites in a common molecule as described in JP 6583136 and JP-A 2020-200311, and onium salts having iodonium cation and carboxylate anion sites in a common molecule as described in JP 6274755.

If a PAG capable of generating a strong acid is an onium salt, an exchange from the strong acid generated upon exposure to high-energy radiation to a weak acid can take place as mentioned above, but it rarely happens that the weak acid generated upon exposure to high-energy radiation collides with the unreacted onium salt capable of generating a strong acid to induce a salt exchange. This is because of the phenomenon that an onium cation is more likely to form an ion pair with a stronger acid anion.

When the onium salt having formula (1) or (2) is used as the quencher (C), the amount of the onium salt used is preferably 0.1 to 20 parts by weight, more preferably 0.1 to 10 parts by weight per 80 parts by weight of the base polymer (A). As long as the amount of onium salt type quencher (C) is in the range, a satisfactory resolution is available without a substantial lowering of sensitivity. The onium salt having formula (1) or (2) may be used alone or in admixture.

Nitrogen-containing compounds may also be used as the quencher (C). Suitable nitrogen-containing compounds include primary, secondary and tertiary amine compounds, specifically amine compounds having a hydroxy group, ether bond, ester bond, lactone ring, cyano group or sulfonate ester bond, as described in JP-A 2008-111103, paragraphs [0146]-[0164] (U.S. Pat. No. 7,537,880), and primary or secondary amine compounds protected with a carbamate group, as described in JP 3790649.

A sulfonic acid sulfonium salt having a nitrogen-containing substituent may also be used as the nitrogen-containing compound. This compound functions as a quencher in the unexposed region, but as a so-called photo-degradable base in the exposed region because it loses the quencher function in the exposed region due to neutralization thereof with the acid generated by itself. Using a photo-degradable base, the contrast between exposed and unexposed regions can be further enhanced. With respect to the photo-degradable base, reference may be made to JP-A 2009-109595 and JP-A 2012-046501, for example.

When the nitrogen-containing compound is used as the quencher (C), the amount of the nitrogen-containing compound used is preferably 0.001 to 12 parts by weight, more preferably 0.01 to 8 parts by weight per 80 parts by weight of the base polymer (A). The nitrogen-containing compound may be used alone or in admixture.

### (D) Photoacid Generator

The chemically amplified resist composition may comprise (D) a photoacid generator (PAG). The PAG is not particularly limited as long as it is capable of generating an acid upon exposure to high-energy radiation. The preferred PAGs are compounds having the formulae (3) and (4).

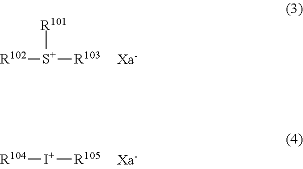

In formula (3), R¹⁰¹ to R¹⁰⁵ are each independently halogen or a C₁-C₂₀ hydrocarbyl group which may contain a heteroatom. Any two of R¹⁰¹, R¹⁰² and R¹⁰³ may bond together to form a ring with the sulfur atom to which they are attached.

Examples of the cation in the sulfonium salt having formula (3) include, but are not limited to, those exemplified above for the sulfonium cations having formulae (Z-1) and (Z-3), those described in JP-A 2024-003744, paragraphs [0102]-[0125], WO 2024/128017, paragraphs [0044]-[0049], and JP 7491173, paragraphs [0035]-[0046]. Examples of the cation in the iodonium salt having formula (4) include those described in JP-A 2024-000259, paragraph [0181], but are not limited thereto.

In formulae (3) and (4), Xa⁻ is an anion of strong acid, which is typically selected from the following formulae (Xa-1) to (Xa-4).

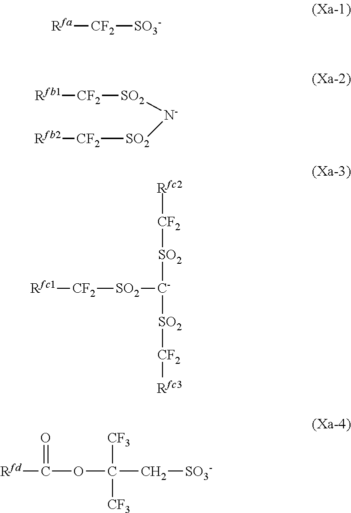

In formula (Xa-1), R^{fa} is fluorine or a C₁-C₄₀ hydrocarbyl group which may contain a heteroatom. The hydrocarbyl group may be saturated or unsaturated and straight, branched or cyclic. Examples thereof are as will be exemplified below for hydrocarbyl group R^{fa1} in formula (Xa-1-1).

Of the anions of formula (Xa-1), a structure having formula (Xa-1-1) is preferred.

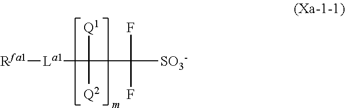

In formula (Xa-1-1), Q¹ and Q² are each independently hydrogen, fluorine, or a C₁-C₆ fluorinated saturated hydrocarbyl group. It is preferred for improving solvent solubility that at least one of Q¹ and Q² be trifluoromethyl. The subscript m is 0, 1, 2, 3 or 4, most preferably m=1. R^{fa1} is a C₁-C₃₅ hydrocarbyl group which may contain a heteroatom. Suitable heteroatoms include oxygen, nitrogen, sulfur and halogen, with oxygen being preferred. Of the hydrocarbyl groups, those of 6 to 30 carbon atoms are preferred because a high resolution is available in fine pattern formation. The hydrocarbyl group R^{fa1} may be saturated or unsaturated and straight, branched or cyclic. Suitable hydrocarbyl groups include C₁-C₃₅ alkyl groups such as methyl, ethyl, n-propyl, isopropyl, n-butyl, isobutyl, sec-butyl, tert-butyl, pentyl, neopentyl, hexyl, heptyl, 2-ethylhexyl, nonyl, undecyl, tridecyl, pentadecyl, heptadecyl, icosyl: C₃-C₃₅ cyclic saturated hydrocarbyl groups such as cyclopentyl, cyclohexyl, 1-adamantyl, 2-adamantyl, 1-adamantylmethyl, norbornyl, norbomylmethyl, tricyclodecyl, tetracyclododecyl, tetracyclododecylmethyl, dicyclohexylmethyl: C₂-C₃₅ unsaturated aliphatic hydrocarbyl groups such as 2-propenyl and 3-cyclohexenyl: C₆-C₃₅ aryl groups such as phenyl, 1-naphthyl, 2-naphthyl and 9-fluorenyl; C₇-C₃₅ aralkyl groups such as benzyl and diphenylmethyl; and combinations thereof.

In the hydrocarbyl groups, some or all of the hydrogen atoms may be substituted by a moiety containing a heteroatom such as oxygen, sulfur, nitrogen or halogen, or some constituent —CH₂— may be replaced by a moiety containing a heteroatom such as oxygen, sulfur or nitrogen, so that the group may contain a hydroxy, fluorine, chlorine, bromine, iodine, cyano, nitro, carbonyl, ether bond, ester bond, sulfonic ester bond, carbonate bond, lactone ring, sultone ring, carboxylic anhydride (—C(═O)—O—C(═O)—) or haloalkyl moiety. Examples of the heteroatom-containing hydrocarbyl group include tetrahydrofuryl, methoxy methyl, ethoxymethyl, methylthiomethyl, acetamidomethyl, trifluoroethyl, (2-methoxyethoxy)methyl, acetoxy methyl, 2-carboxy-1-cyclohexyl, 2-oxopropyl, 4-oxo-1-adamantyl, and 3-oxocyclohexyl.

In formula (Xa-1-1), Lal is a single bond, ether bond, ester bond, sulfonate ester bond, carbonate bond or carbamate bond. From the aspect of synthesis, Lal is preferably an ether bond or ester bond, more preferably ester bond.

Examples of the anion having formula (Xa-1) are shown below, but not limited thereto. Herein Q¹ is as defined above.

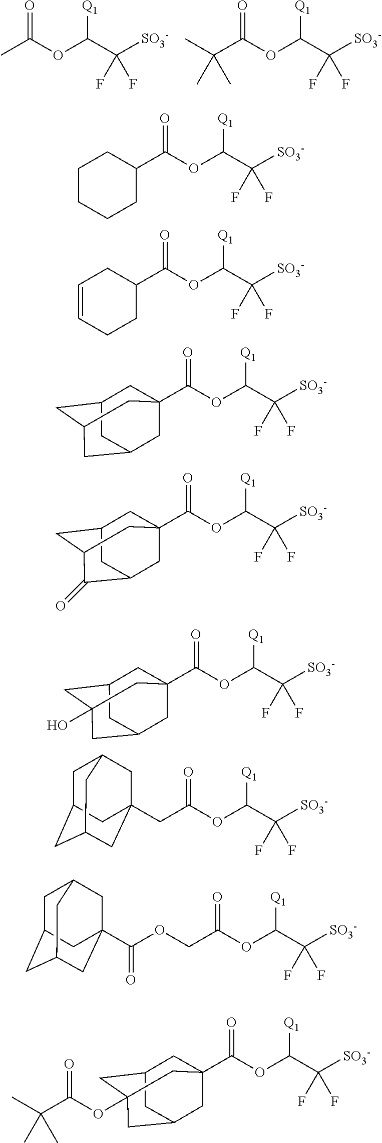

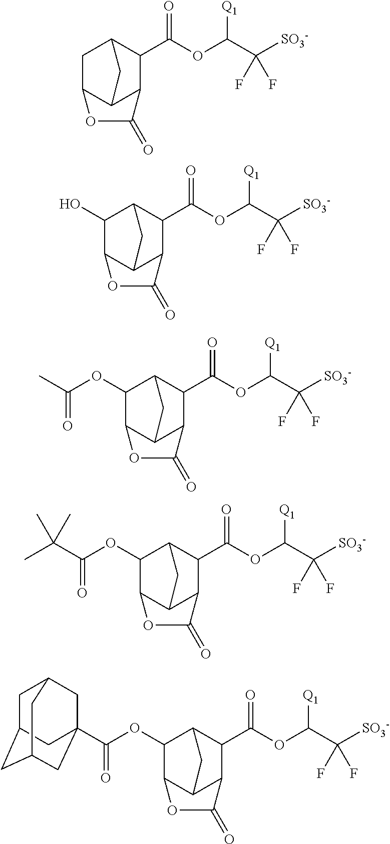

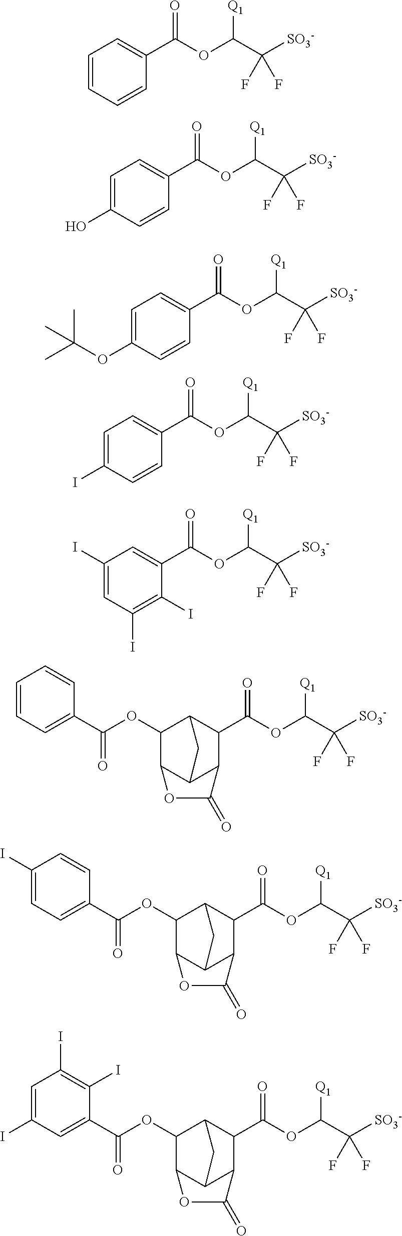

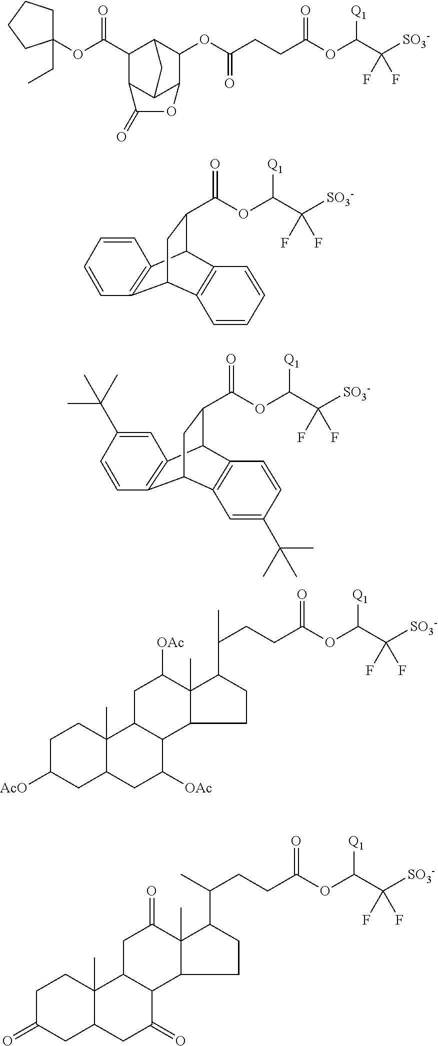

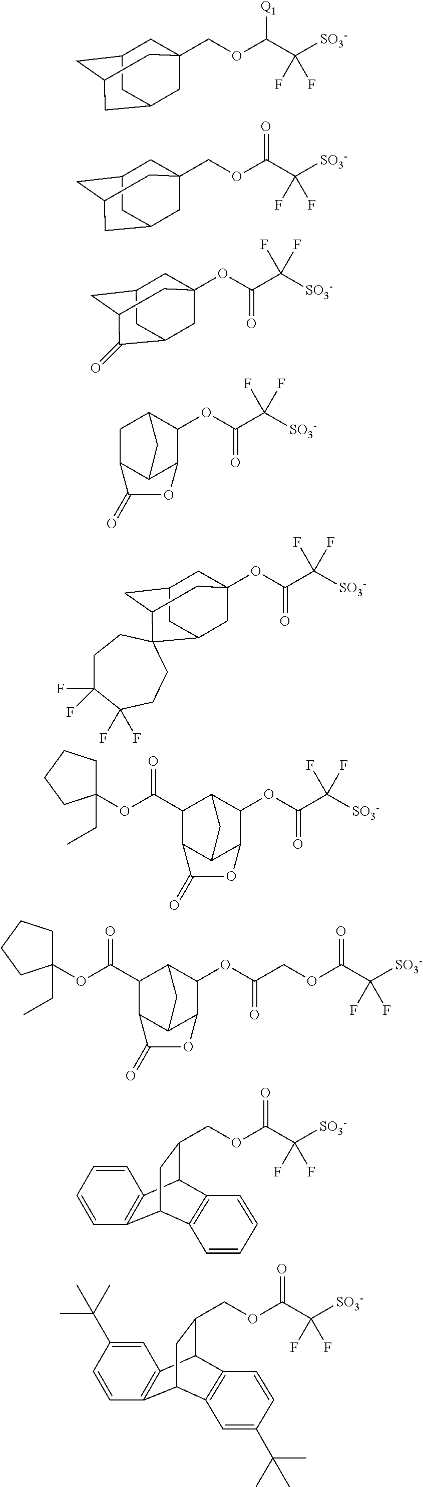

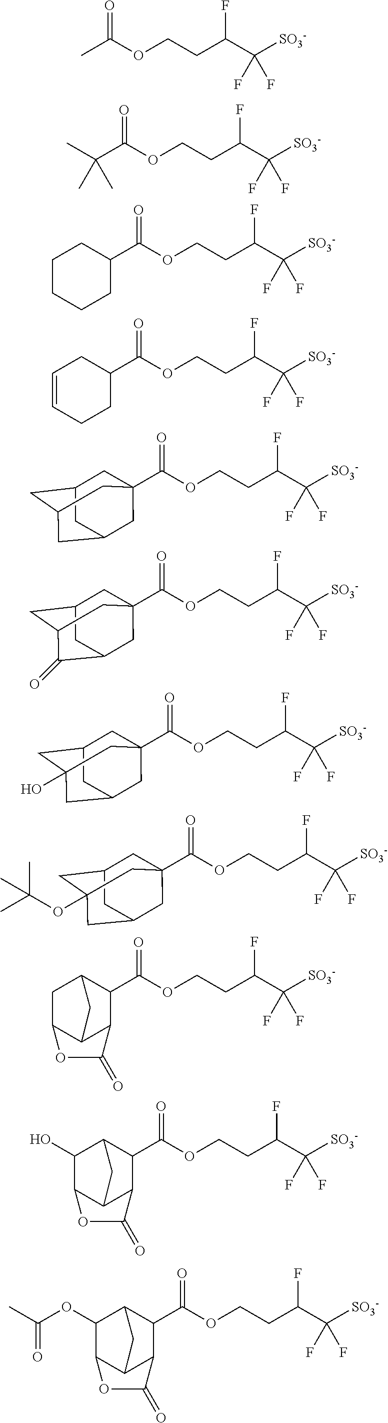

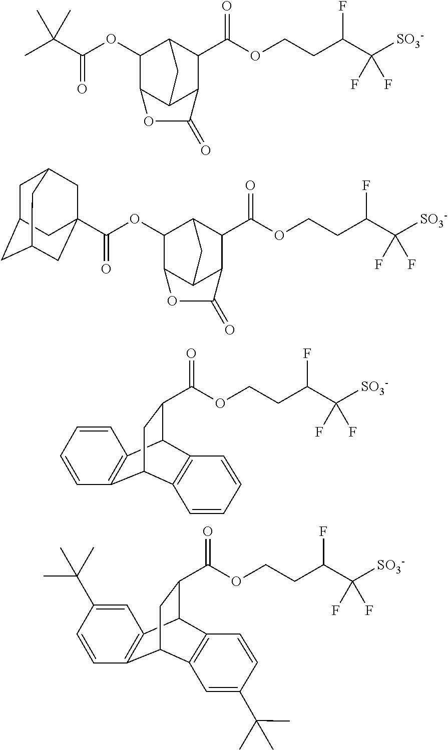

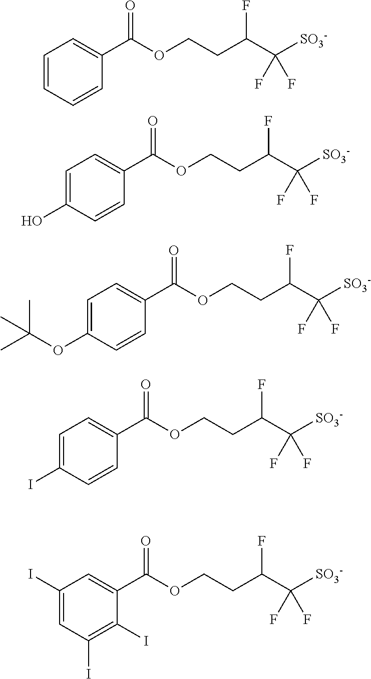

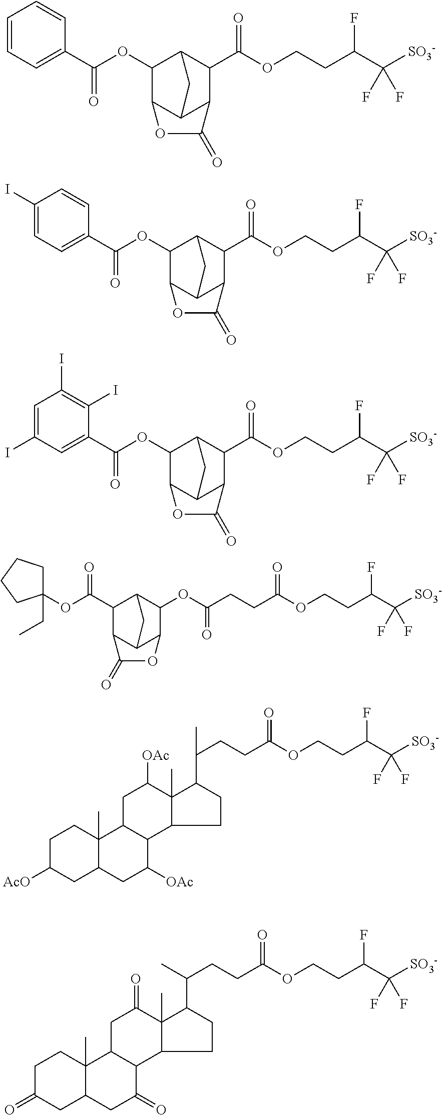

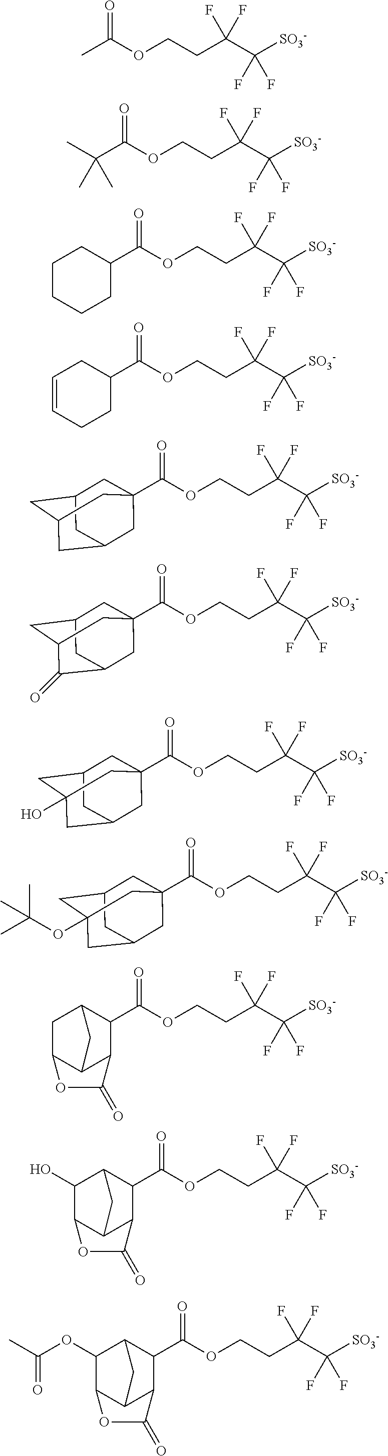

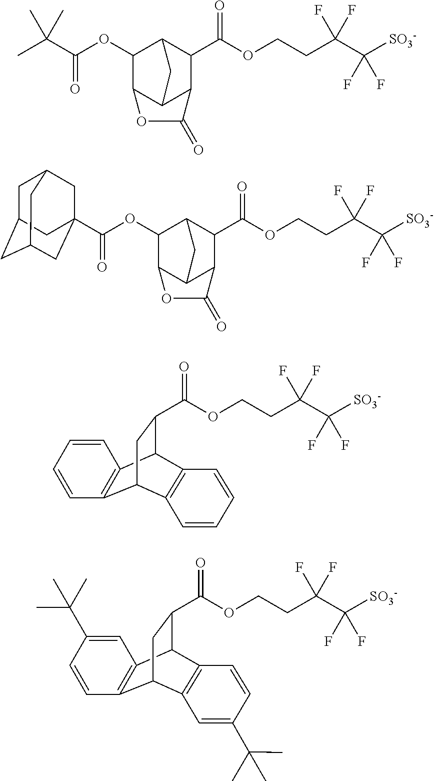

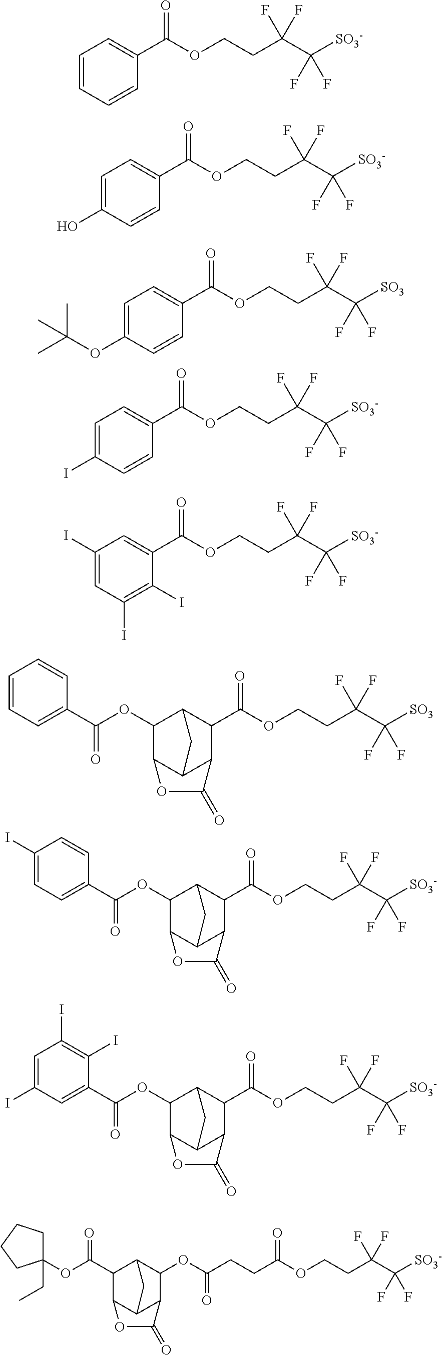

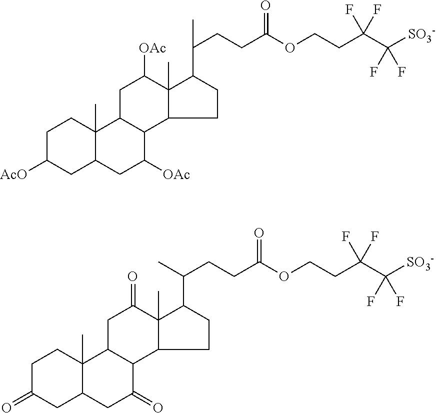

In formula (Xa-2), R^{fb1} and R^{fb2} are each independently fluorine or a C₁-C₄₀ hydrocarbyl group which may contain a heteroatom. The hydrocarbyl group may be saturated or unsaturated and straight, branched or cyclic. Examples thereof are as exemplified above for the hydrocarbyl group R^{fa1} in formula (Xa-1-1). R^{fb1} and R^{fb2} are preferably fluorine or a C₁-C₄ straight fluorinated alkyl group. A pair of R^{fb1} and R^{fb2} may bond together to form a ring with the linkage (—CF₂—SO₂—N⁻—SO₂—CF₂—) to which they are attached, and the R^{fb1}-R^{fb2} group is preferably a fluorinated ethylene or fluorinated propylene group.

In formula (Xa-3), R^{fc1}, R^{fc2} and R^{fc3} are each independently fluorine or a C₁-C₄₀ hydrocarbyl group which may contain a heteroatom. The hydrocarbyl group may be saturated or unsaturated and straight, branched or cyclic. Suitable hydrocarbyl groups are as exemplified above for the hydrocarbyl group R^{fa1} in formula (Xa-1-1). Preferably R^{fc1}, R^{fc2} and R^{fc3} each are fluorine or a straight C₁-C₄ fluorinated alkyl group. A pair of R^{fc1} and R^{fc2} may bond together to form a ring with the linkage (—CF₂—SO₂—C⁻—SO₂—CF₂—) to which they are attached, and the R^{fc1}-R^{fc2} group is preferably a fluorinated ethylene or fluorinated propylene group.

In formula (Xa-4), R^{fd} is a C₁-C₄₀ hydrocarbyl group which may contain a heteroatom. The hydrocarbyl group may be saturated or unsaturated and straight, branched or cyclic. Suitable hydrocarbyl groups are as exemplified above for R^{fa1}.

Examples of the anion having formula (Xa-4) are shown below, but not limited thereto.

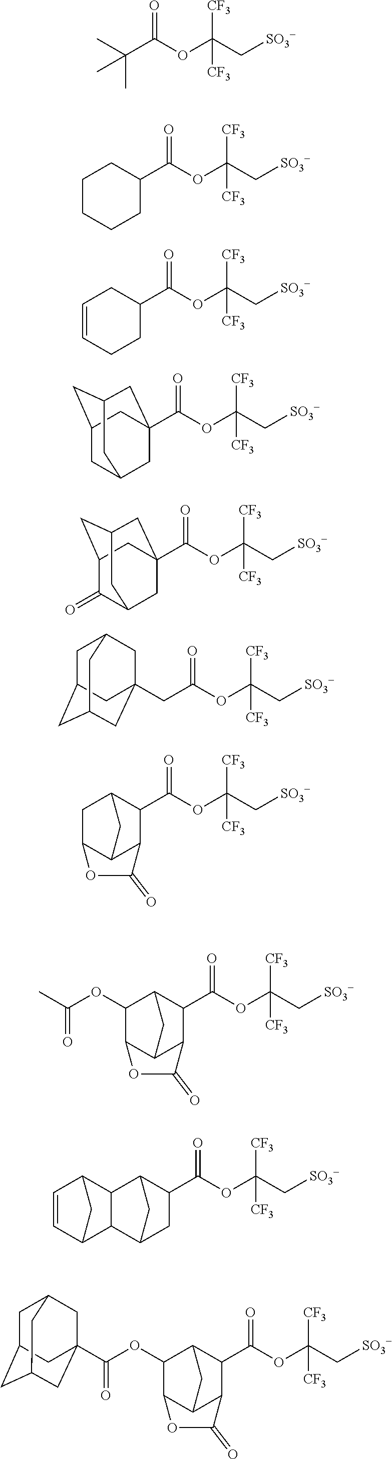

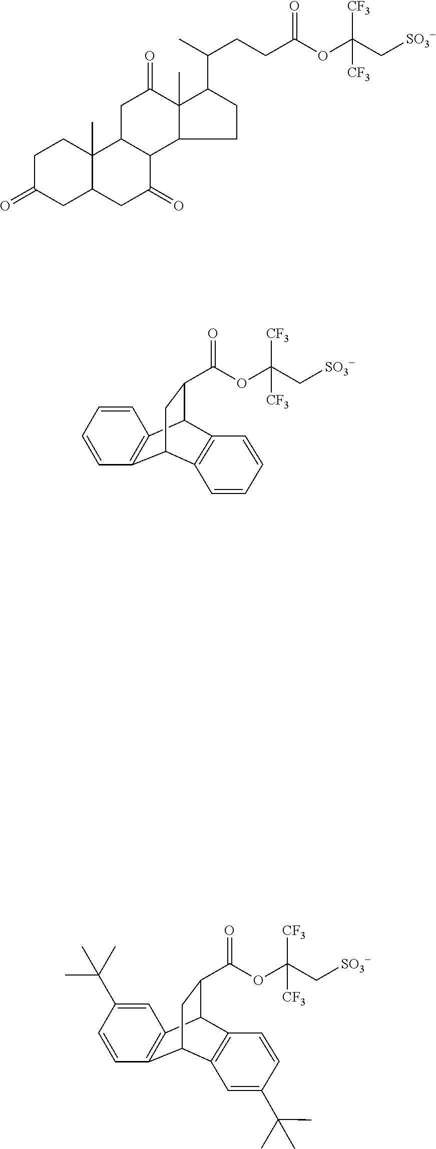

Anions having an iodized or brominated aromatic ring are also useful as the non-nucleophilic counter ion. These anions have the formula (Xa-5).

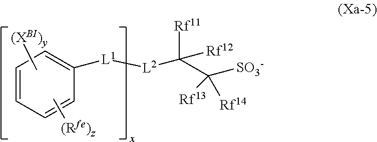

In formula (Xa-5), x is 1, 2 or 3, y is 1, 2, 3, 4 or 5, z is 0, 1, 2 or 3, and y+z is from 1 to 5. Preferably, y is 1, 2 or 3, more preferably 2 or 3, and z is 0, 1 or 2.

In formula (Xa-5), X^{BI} is iodine or bromine. A plurality of X^{BI} may be identical or different when x and/or y is 2 or more.

In formula (Xa-5), L¹ is a single bond, ether bond, ester bond, or a C₁-C₆ saturated hydrocarbylene group which may contain an ether bond or ester bond. The saturated hydrocarbylene group may be straight, branched or cyclic.

In formula (Xa-5), L² is a single bond or a C₁-C₂₀ divalent linking group when x=1. L² is a C₁-C₂₀ (x+1)-valent linking group when x=2 or 3. The linking group may contain an oxygen, sulfur or nitrogen atom.

In formula (Xa-5), R^{fe} is hydroxy, carboxy, fluorine, chlorine, bromine, amino group, or a C₁-C₂₀ hydrocarbyl, C₁-C₂₀ hydrocarbyloxy, C₂-C₂₀ hydrocarbylcarbonyl, C₂-C₂₀ hydrocarbyloxycarbonyl, C₂-C₂₀ hydrocarbylcarbonyloxy, or C₁-C₂₀ hydrocarbylsulfonyloxy group, which may contain fluorine, chlorine, bromine, hydroxy, amino or ether bond, or —N(R^{feA})(R^{feB}), —N(R^{feC})—C(═O)—R^{feD} or —N(R^{feC})—C(═O)—O—R^{feD}. R^{feA} and R^{feB} are each independently hydrogen or a C₁-C₆ saturated hydrocarbyl group. R^{feC} is hydrogen, or a C₁-C₆ saturated hydrocarbyl group which may contain halogen, hydroxy, C₁-C₆ saturated hydrocarbyloxy, C₂-C₆ saturated hydrocarbylcarbonyl or C₂-C₆ saturated hydrocarbylcarbonyloxy moiety. R^{feD} is a C₁-C₁₆ aliphatic hydrocarbyl group, C₆-C₁₂ aryl group or C₇-Cis aralkyl group, which may contain halogen, hydroxy, C₁-C₆ saturated hydrocarbyloxy, C₂-C₆ saturated hydrocarbylcarbonyl or C₂-C₆ saturated hydrocarbylcarbonyloxy moiety. The aliphatic hydrocarbyl group may be saturated or unsaturated and straight, branched or cyclic. The hydrocarbyl, hydrocarbyloxy, hydrocarbylcarbonyl, hydrocarbyloxycarbonyl, hydrocarbylcarbonyloxy, and hydrocarbylsulfonyloxy groups may be straight, branched or cyclic. A plurality of R^{fe} may be identical or different when x and/or z is 2 or more.

Of these, R^{fe} is preferably hydroxy, —N(R^{feC})—C(═O)—R^{feD}, —N(R^{feC})—C(═O)—O—R^{feD}, fluorine, chlorine, bromine, methyl or methoxy.

In formula (Xa-5), Rf¹¹ to Rf¹⁴ are each independently hydrogen, fluorine or trifluoromethyl, at least one of Rf¹¹ to Rf¹⁴ is fluorine or trifluoromethyl. Rf¹¹ and Rf¹², taken together, may form a carbonyl group. More preferably, both Rf¹³ and Rf¹⁴ are fluorine.

Examples of the anion having formula (Xa-5) are shown below, but not limited thereto. X^{BI} is as defined above.

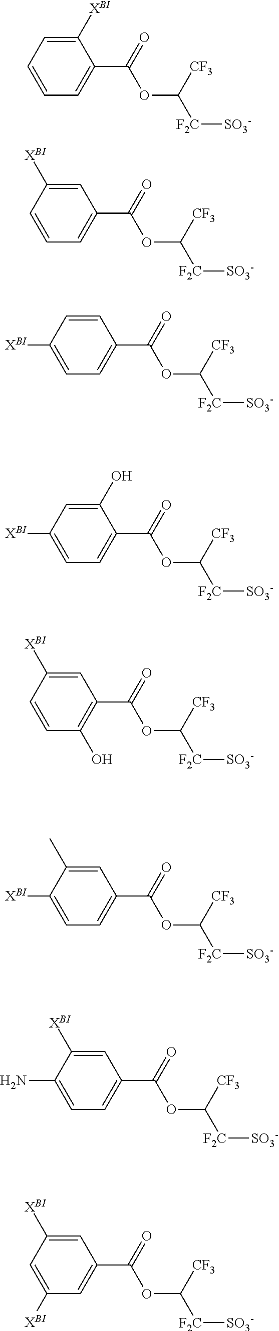

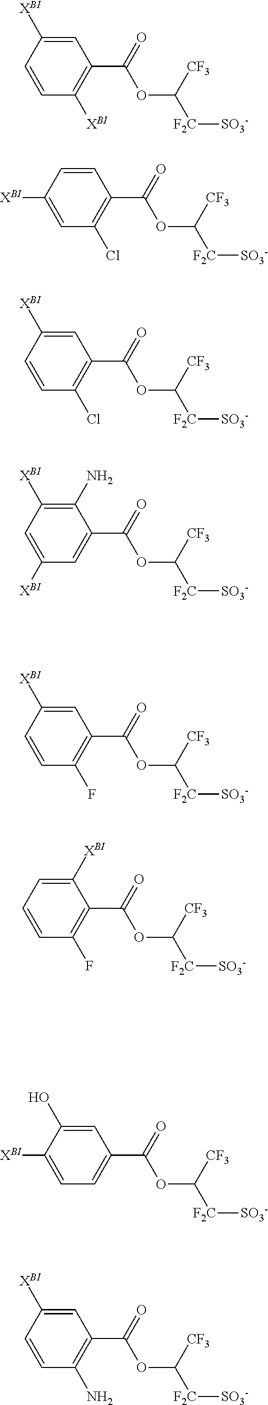

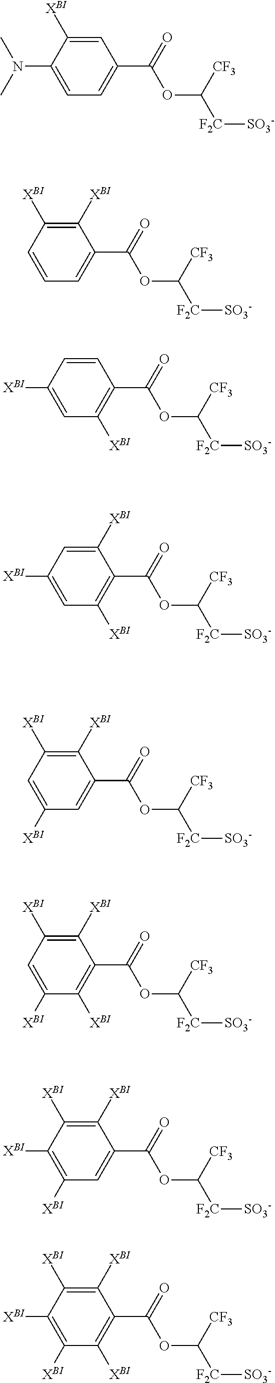

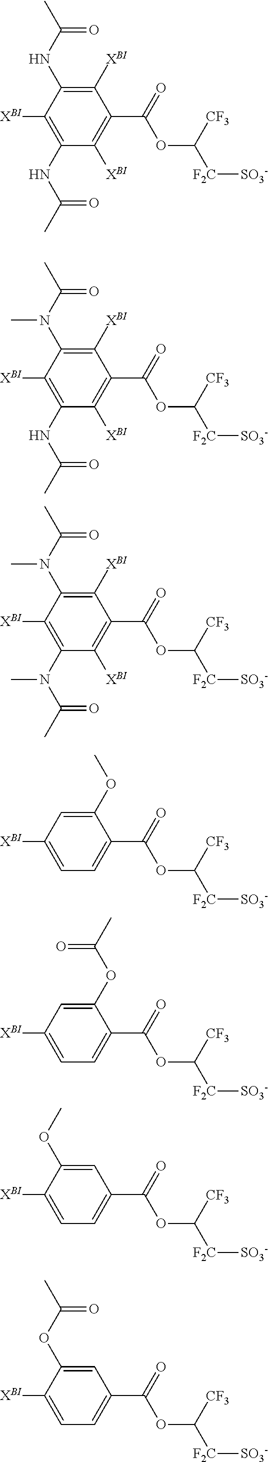

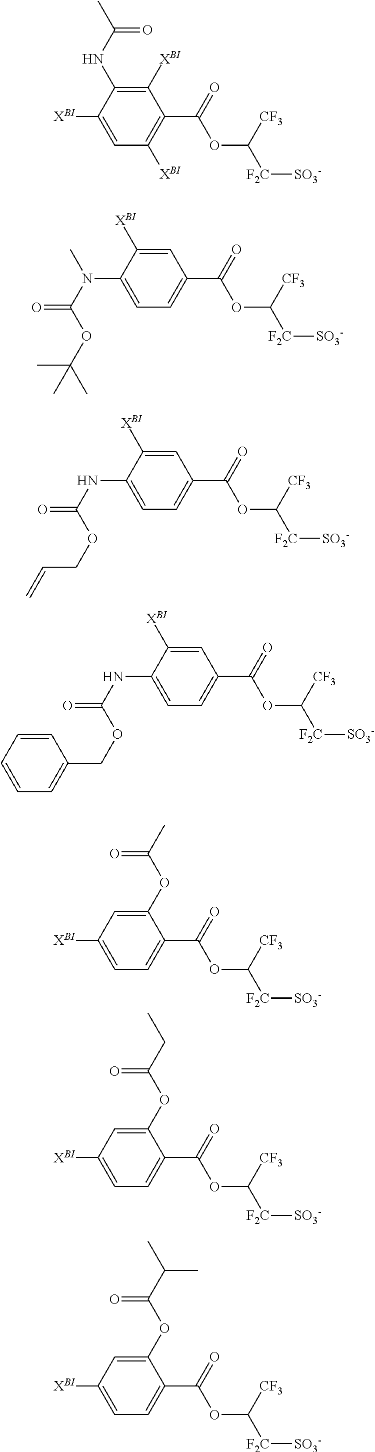

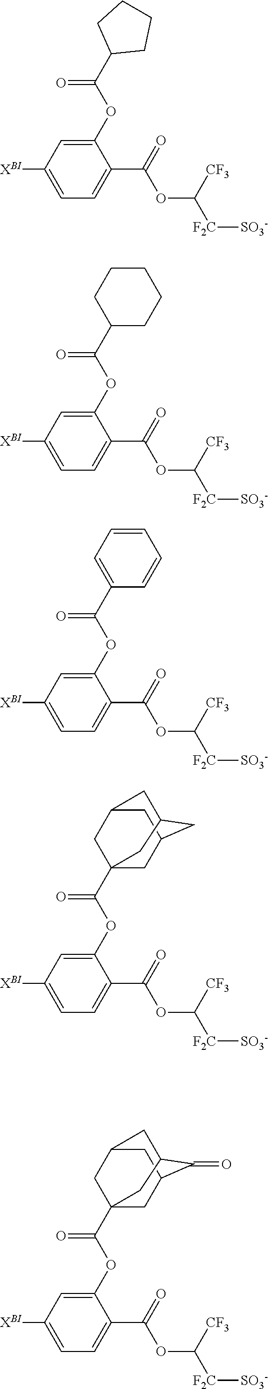

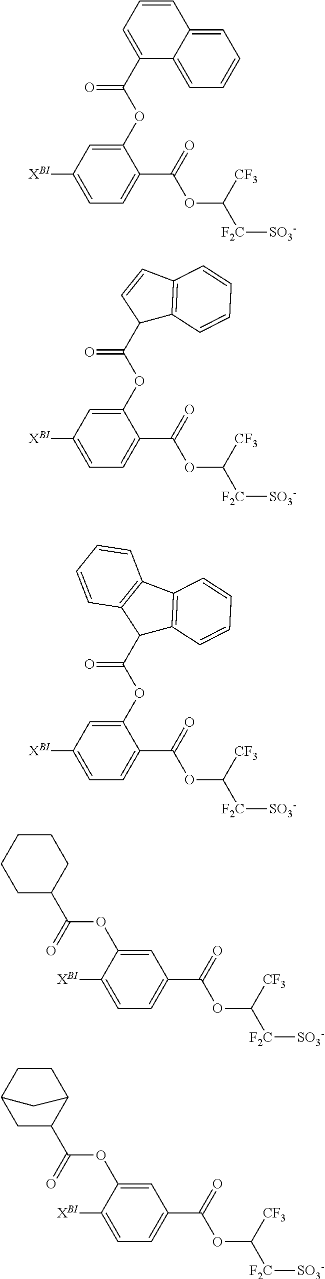

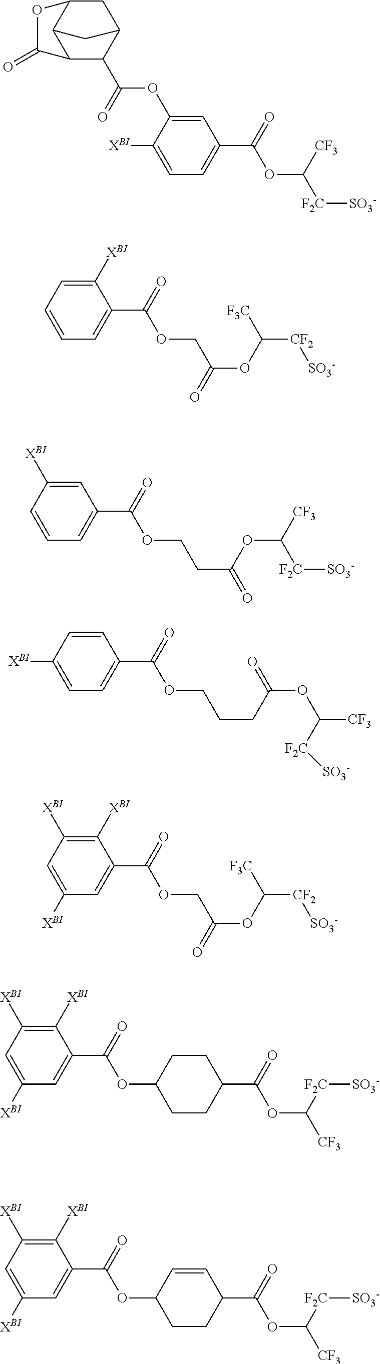

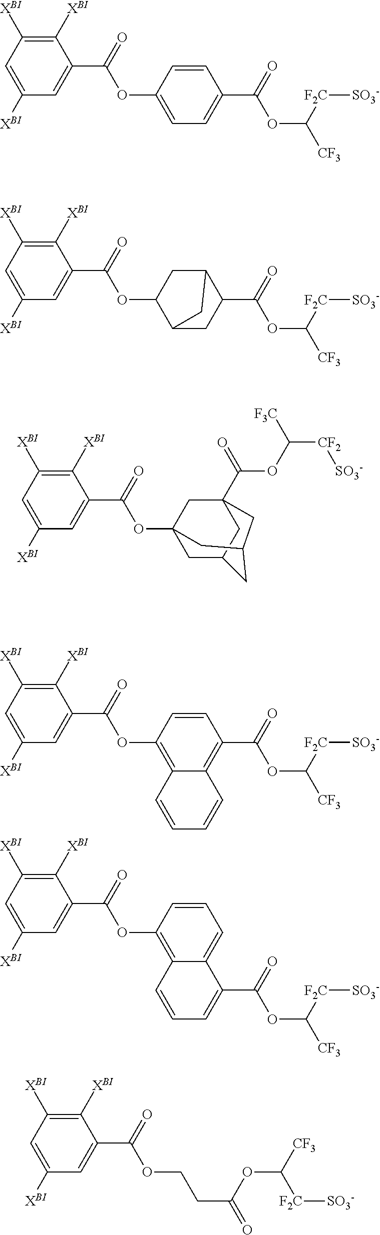

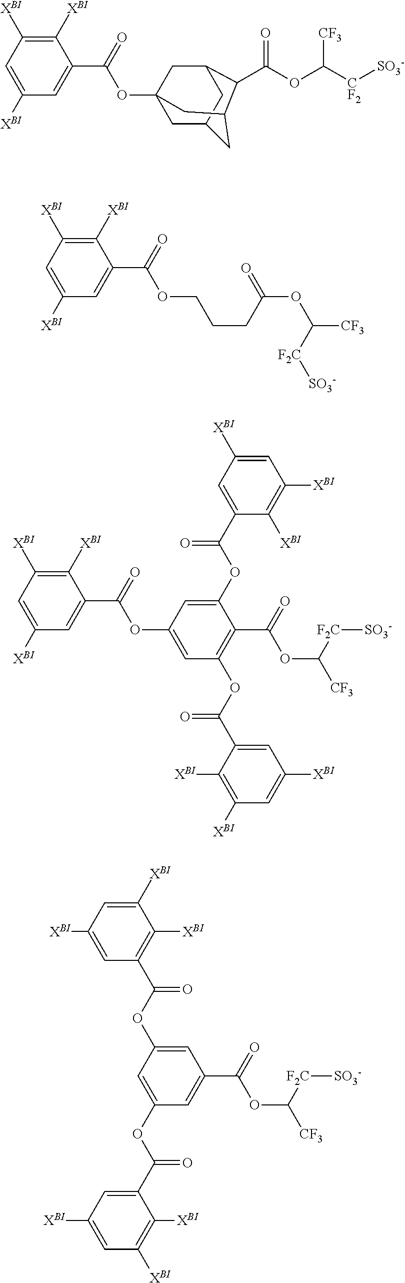

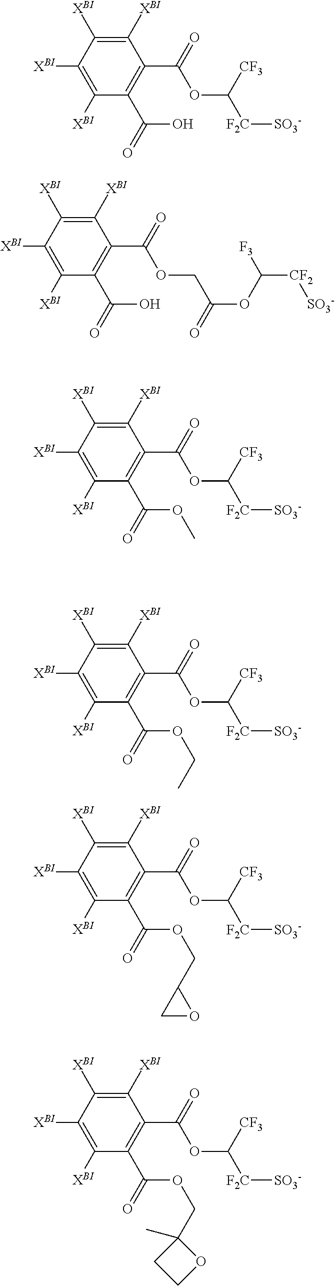

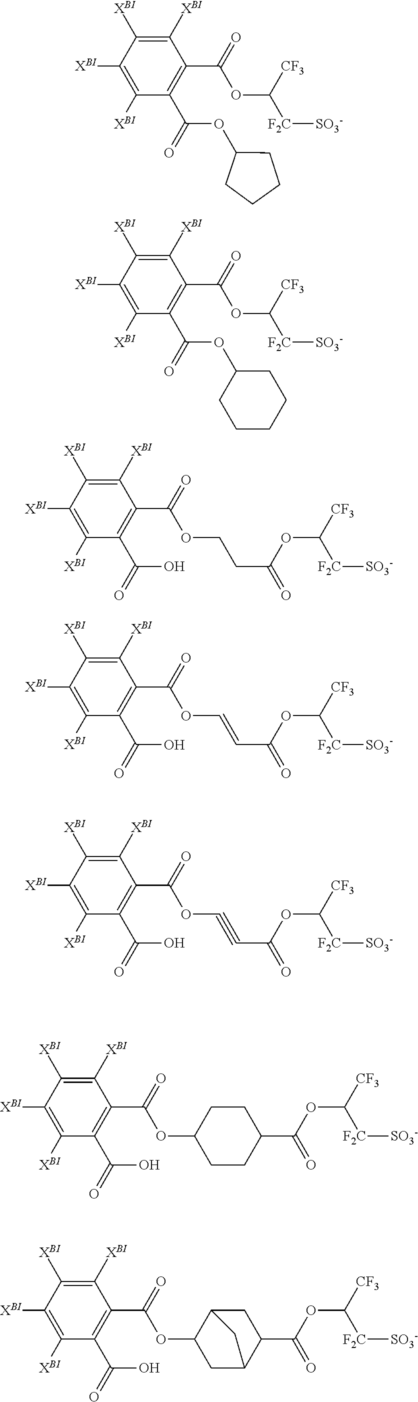

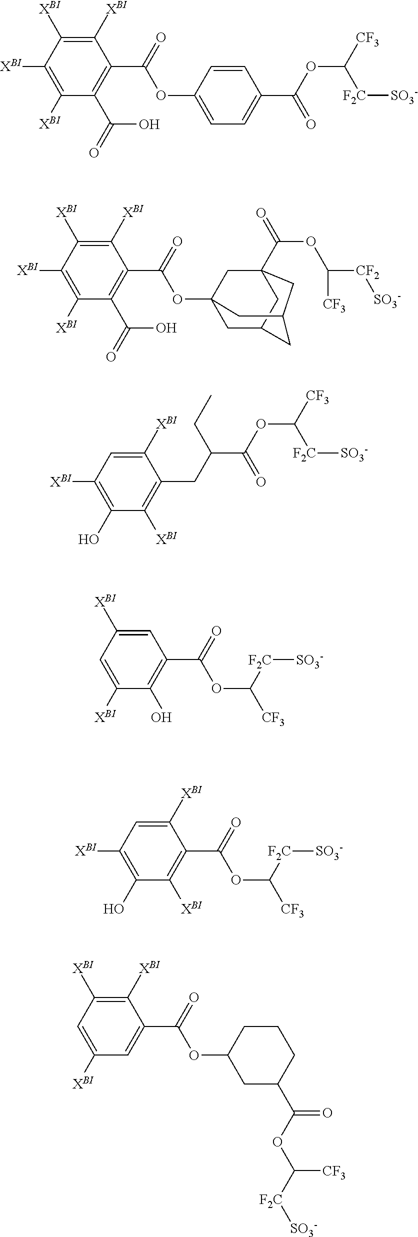

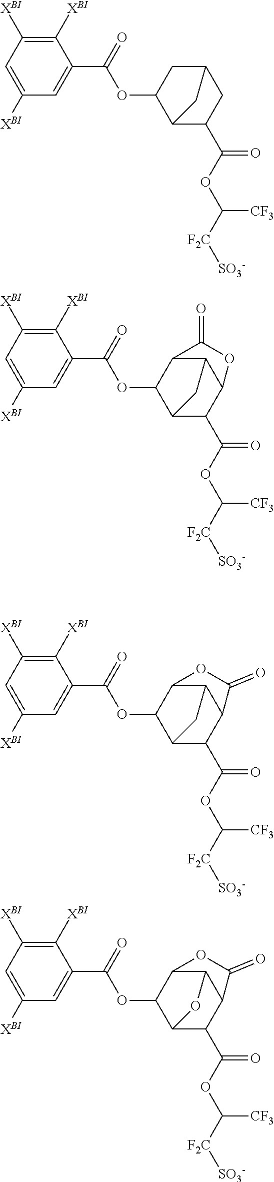

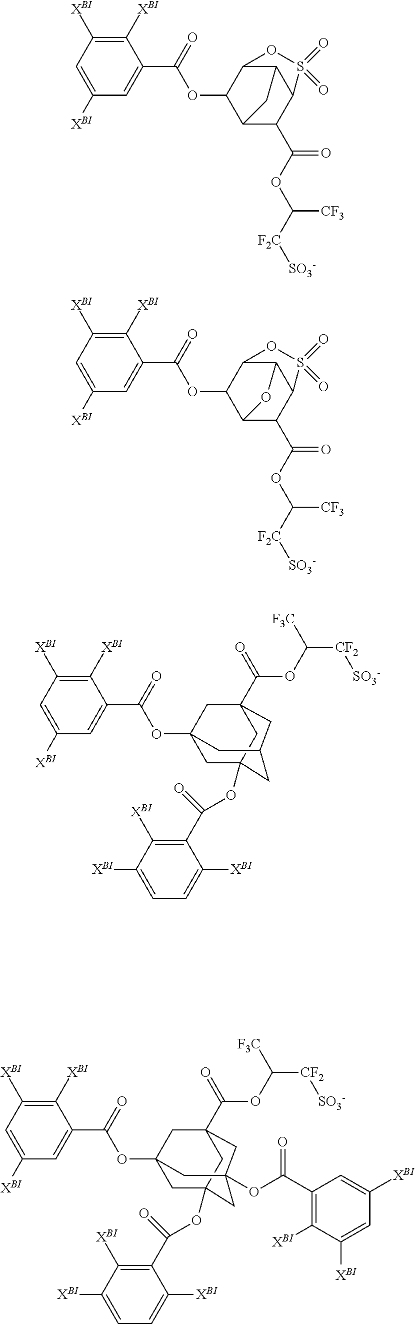

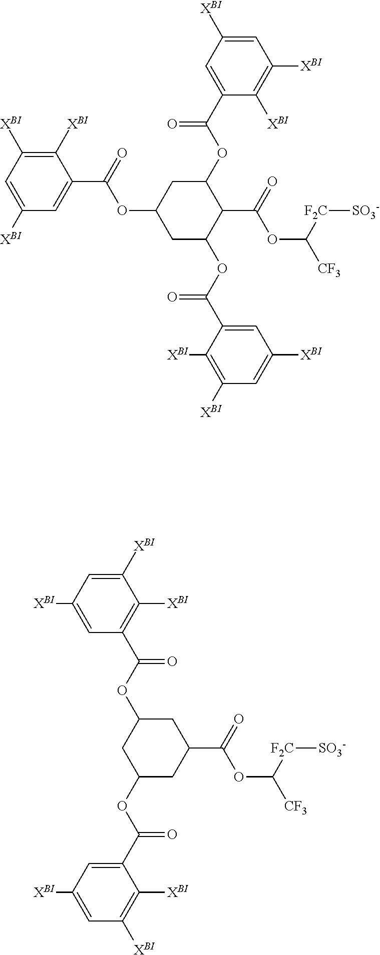

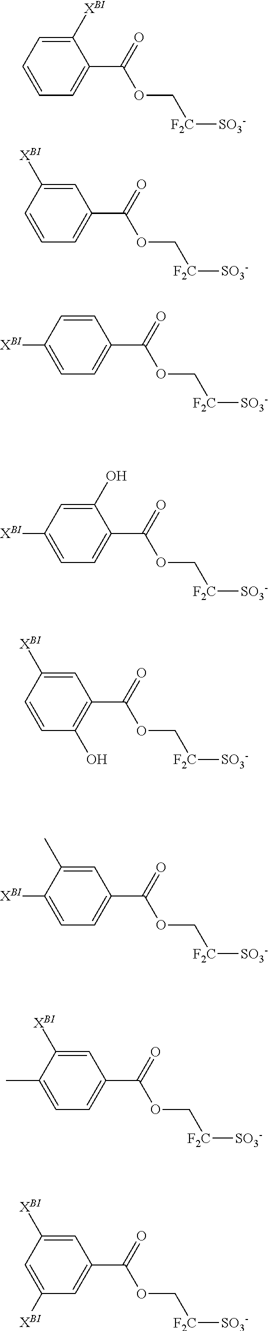

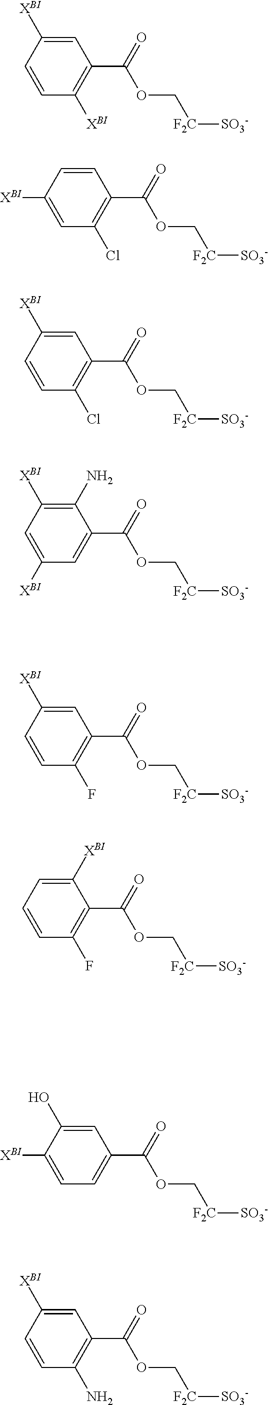

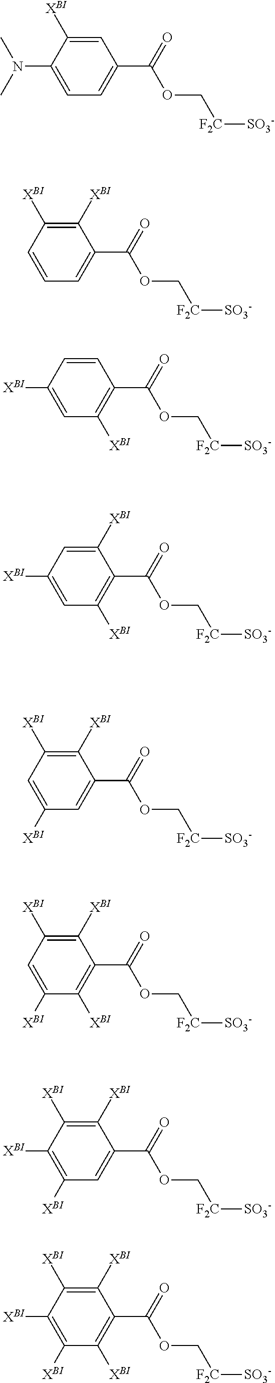

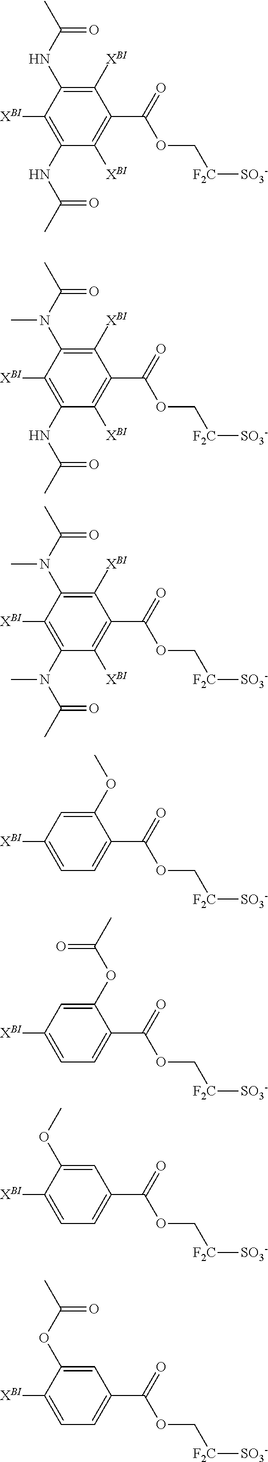

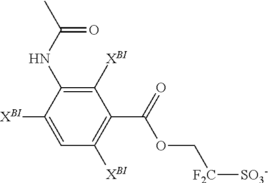

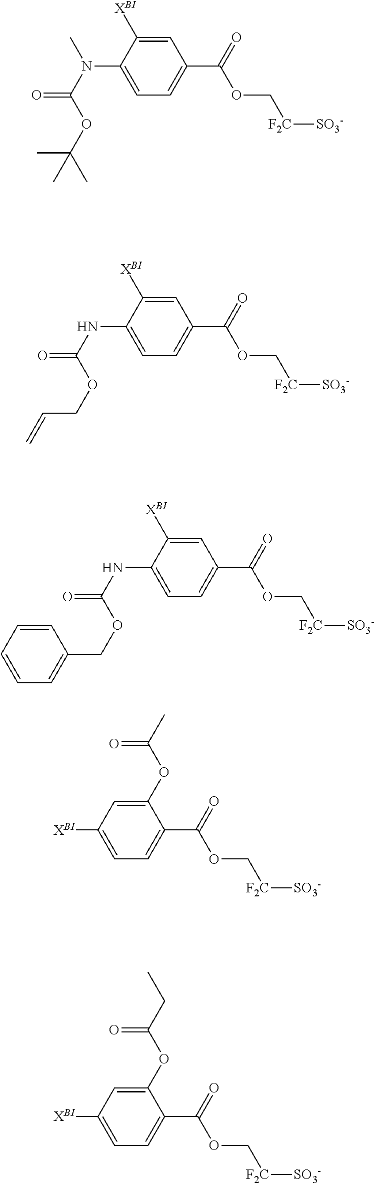

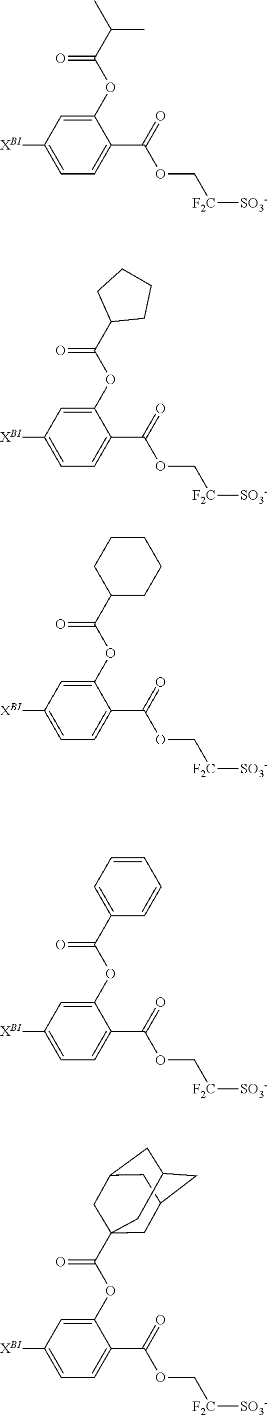

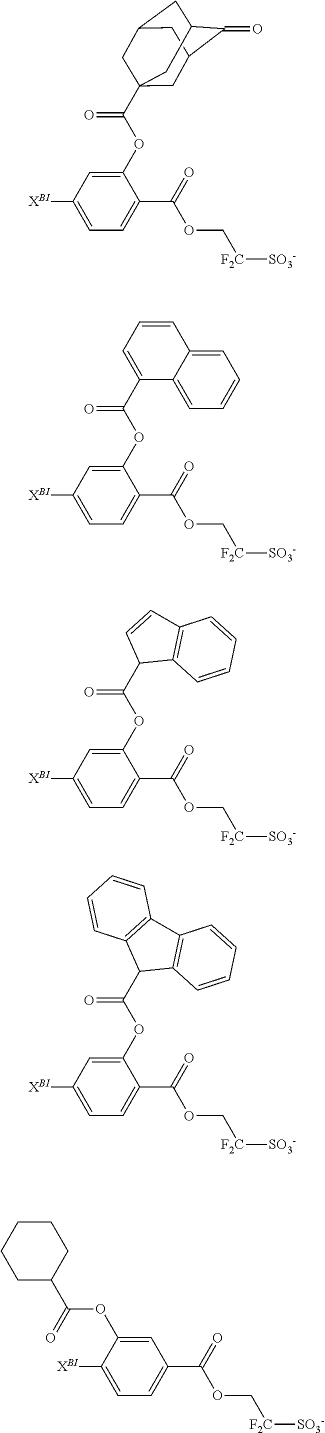

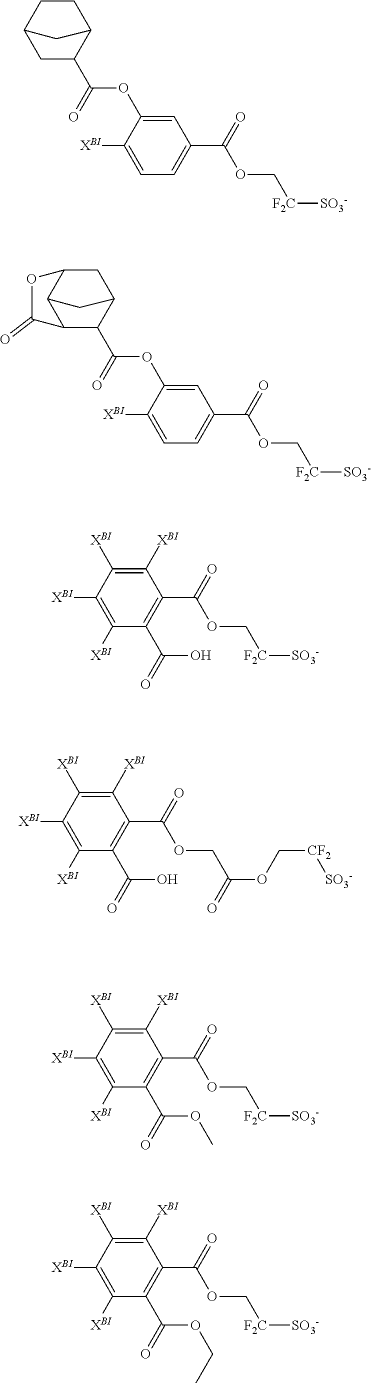

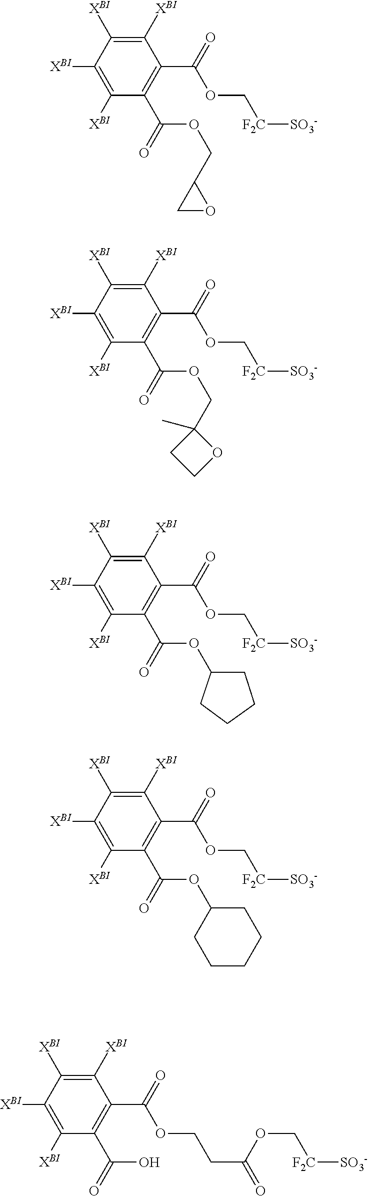

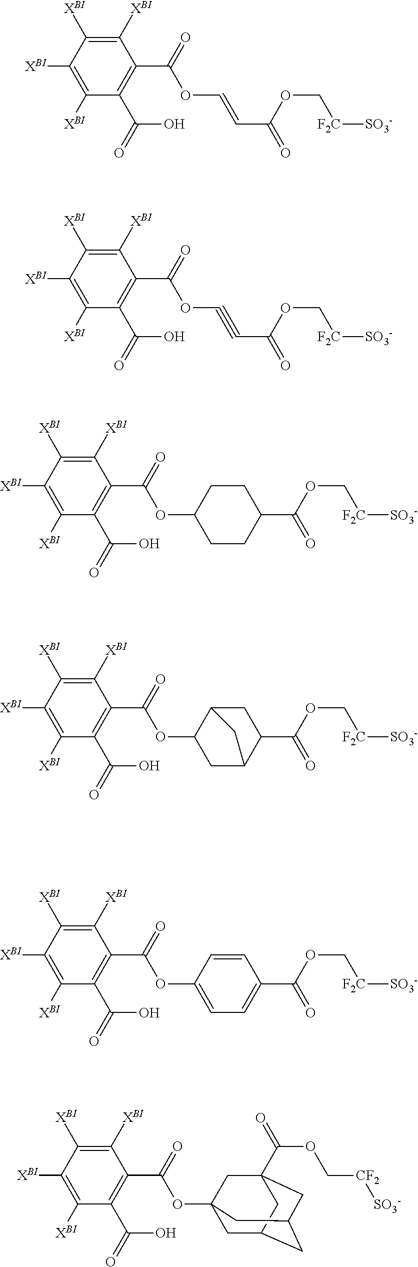

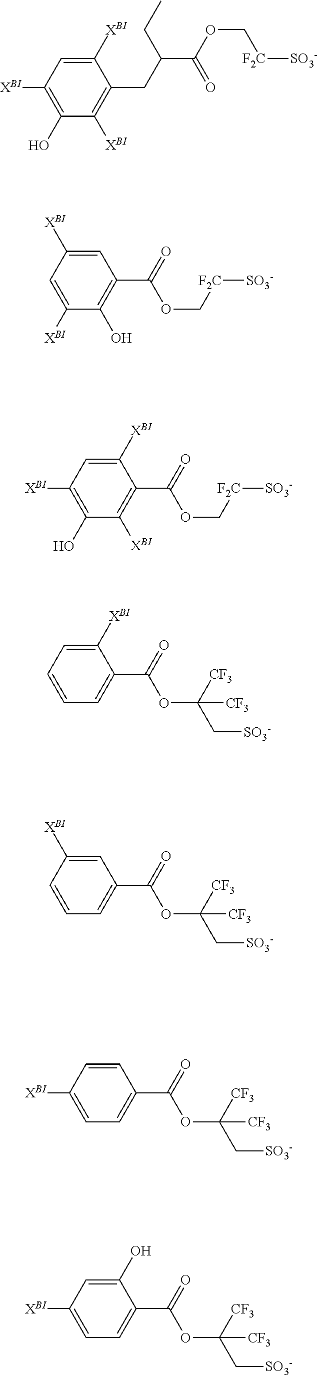

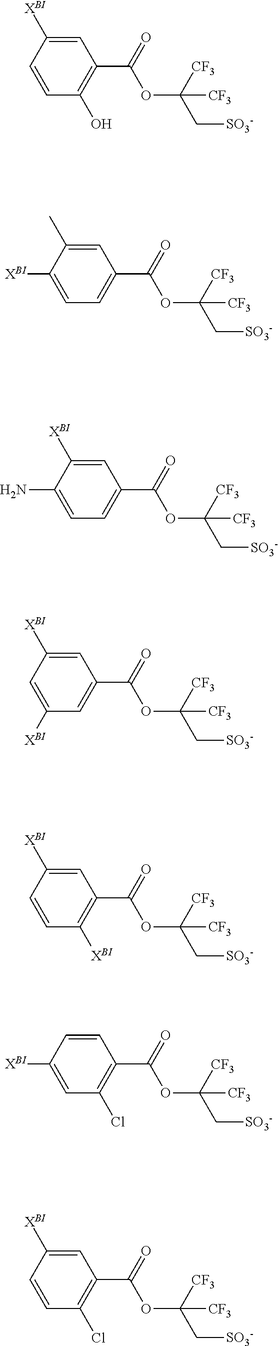

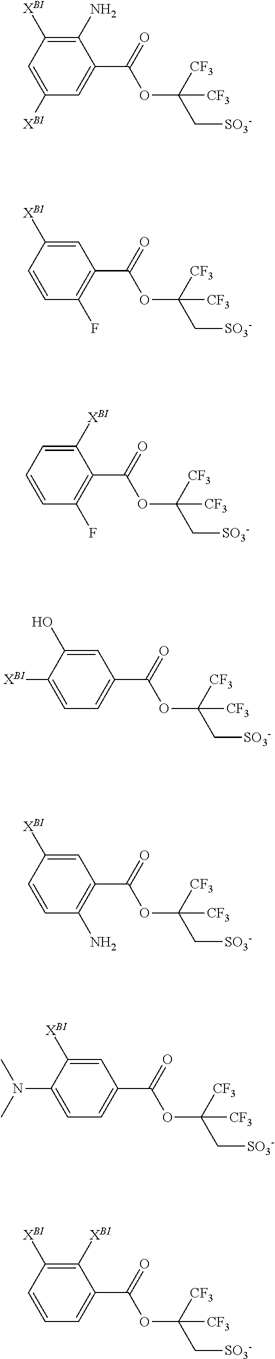

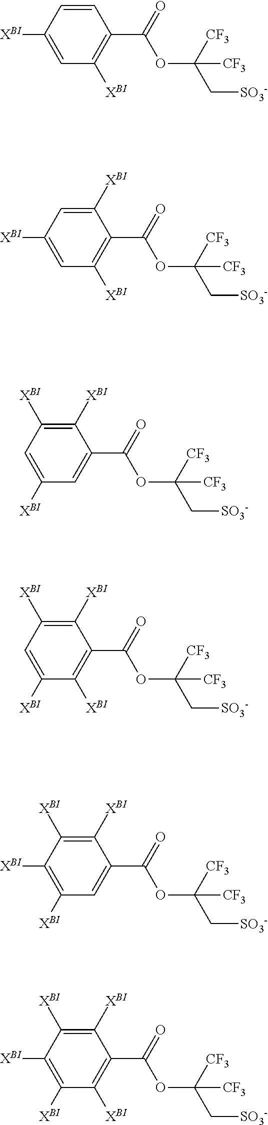

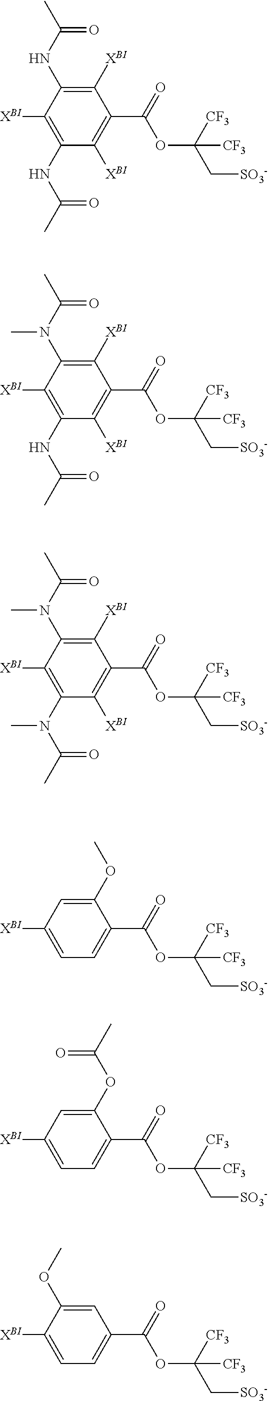

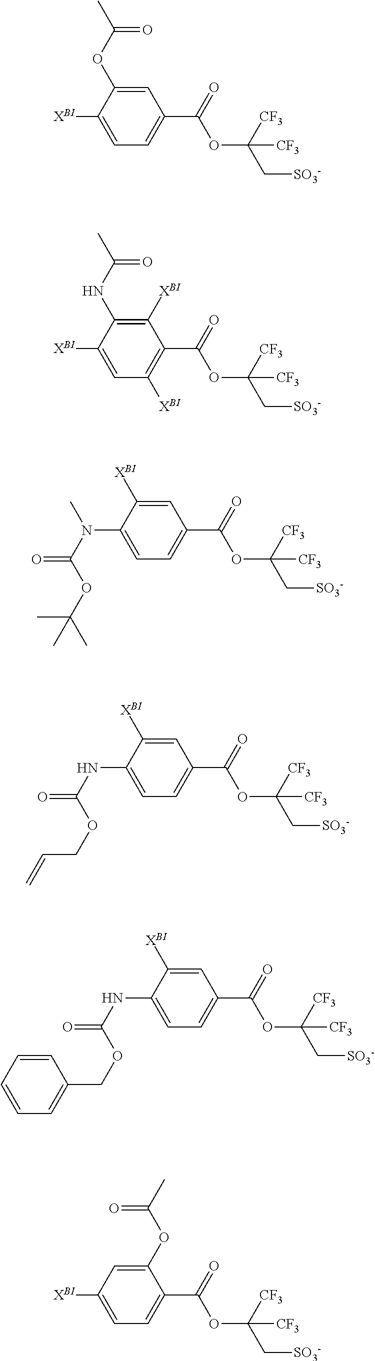

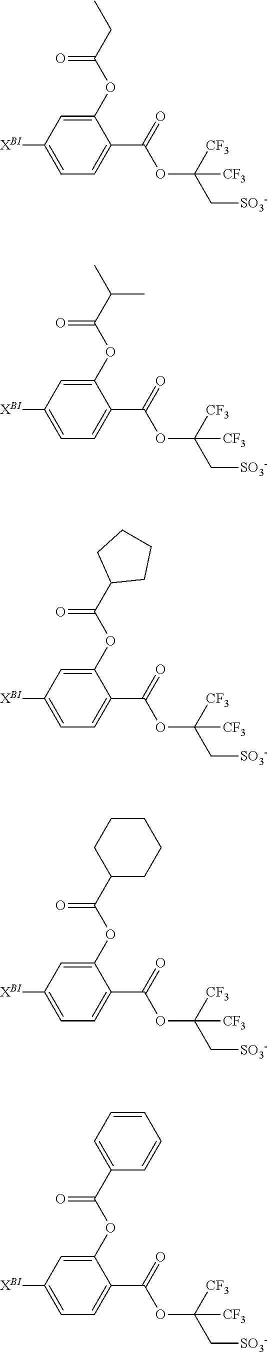

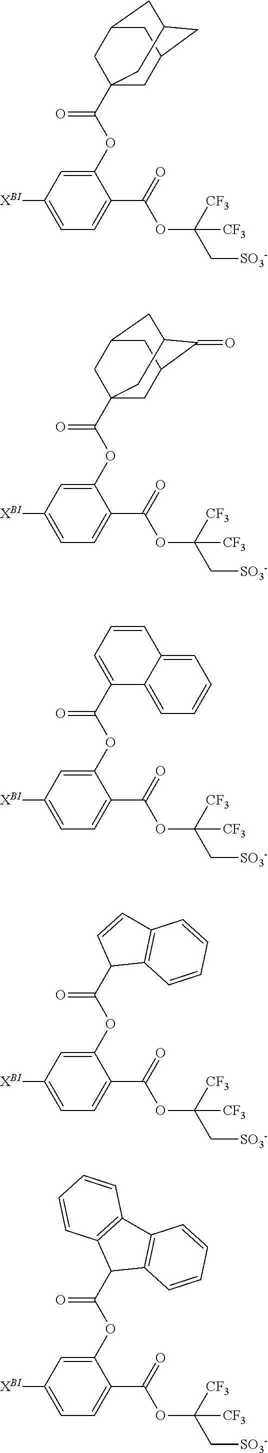

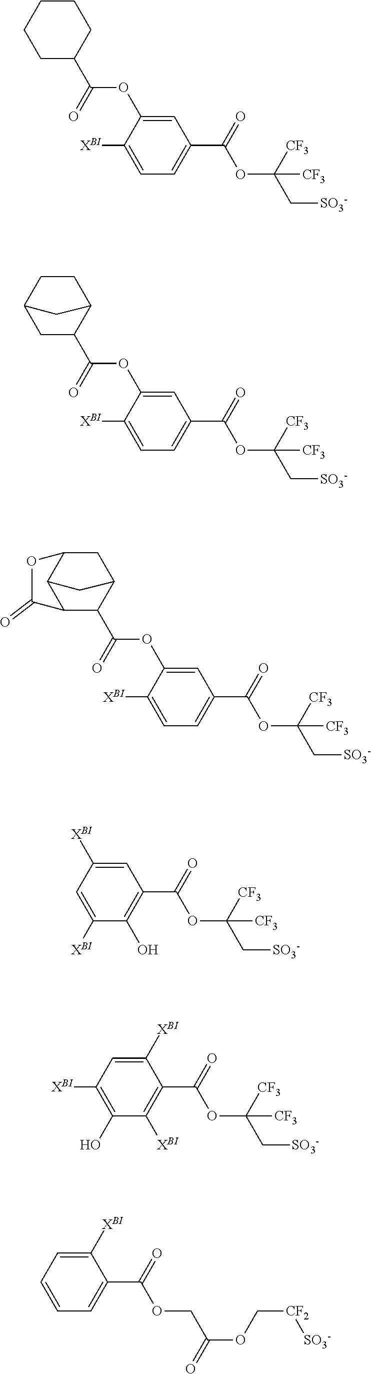

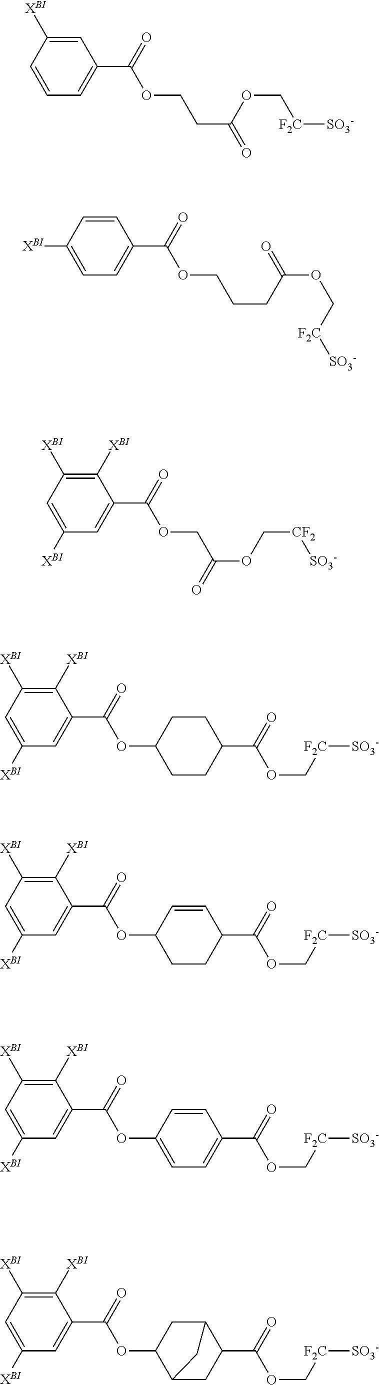

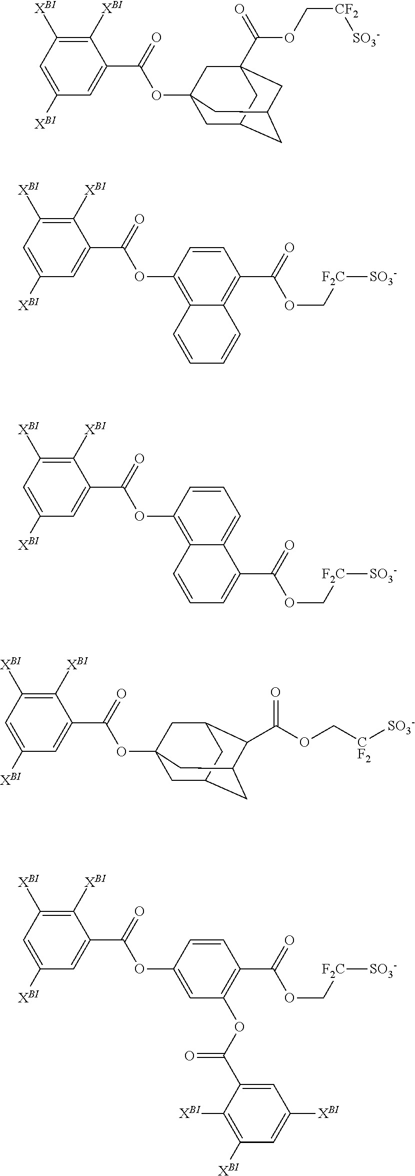

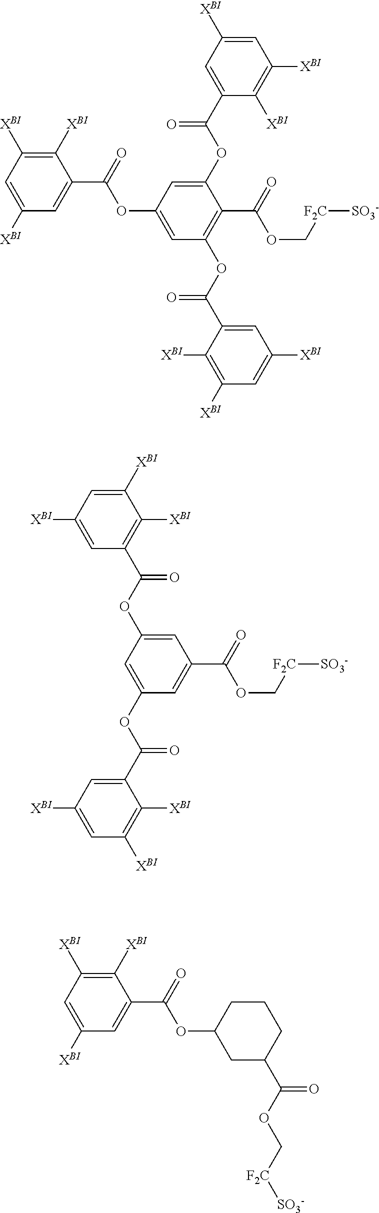

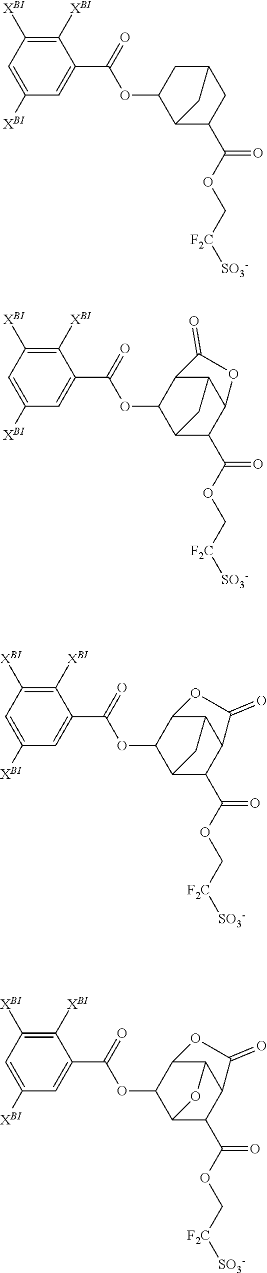

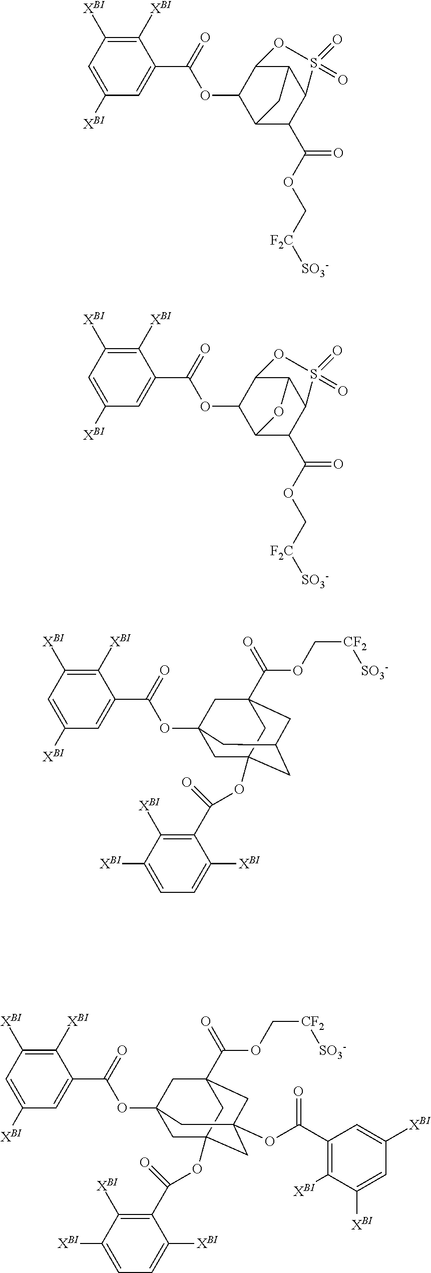

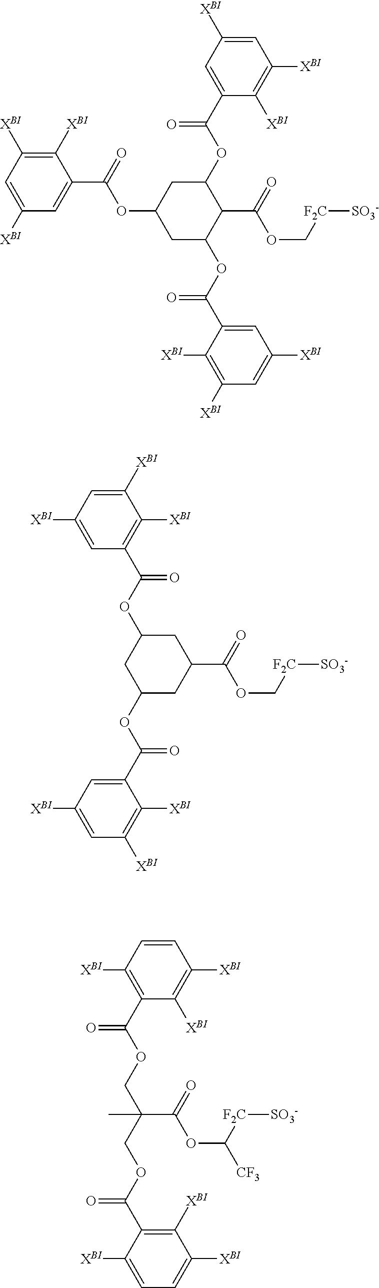

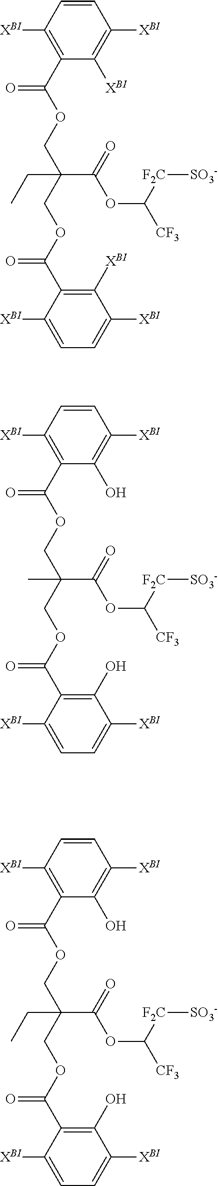

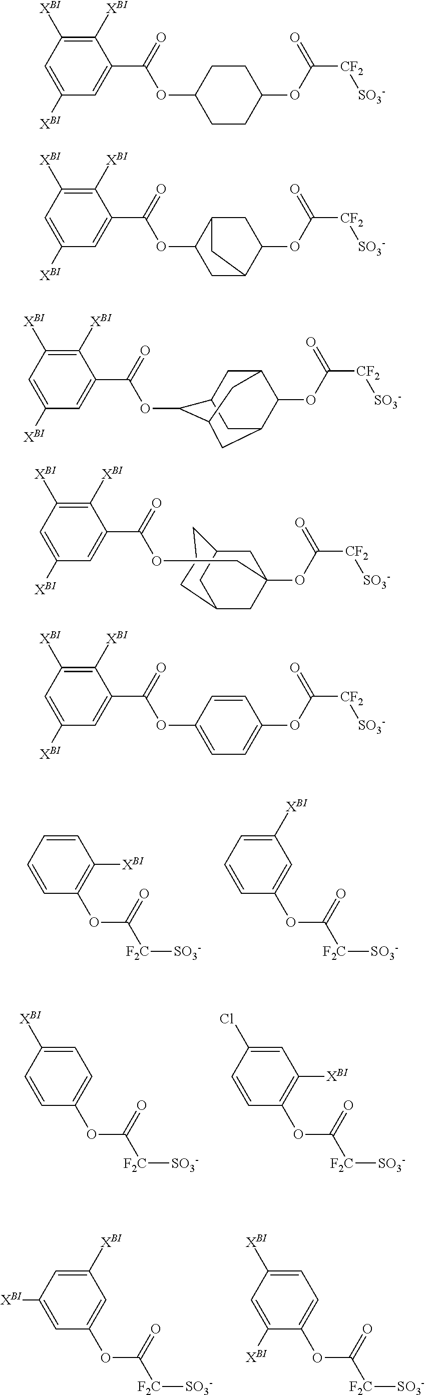

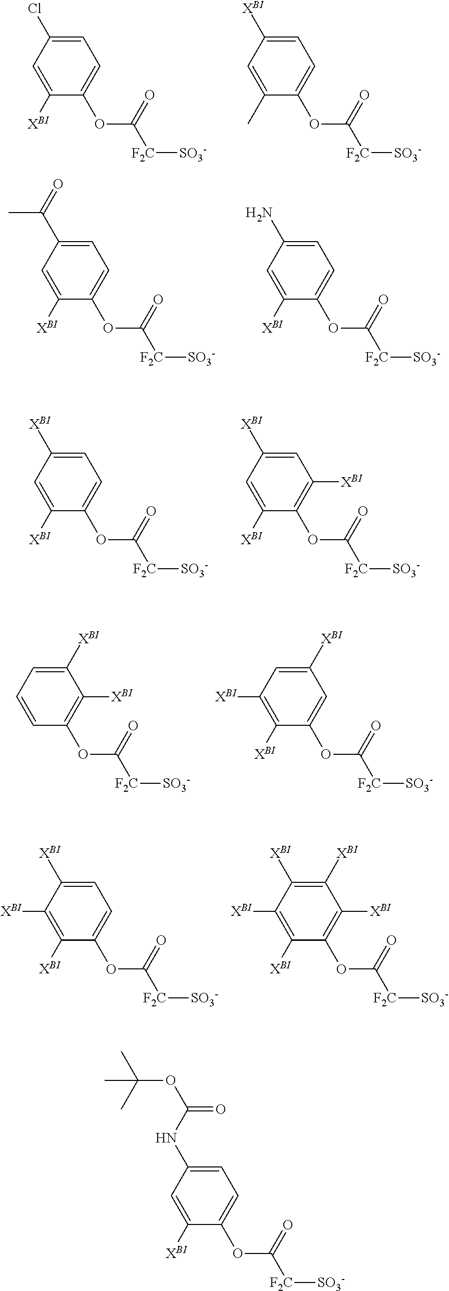

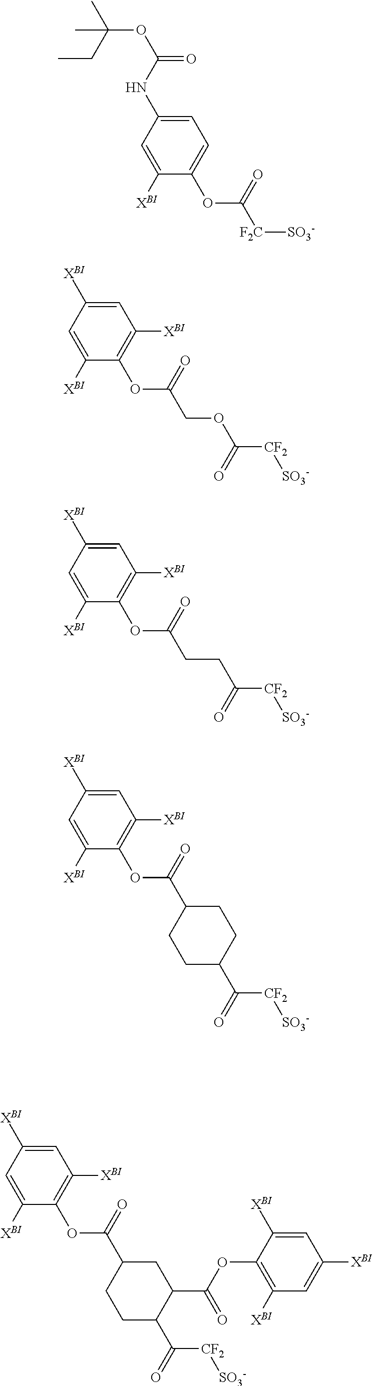

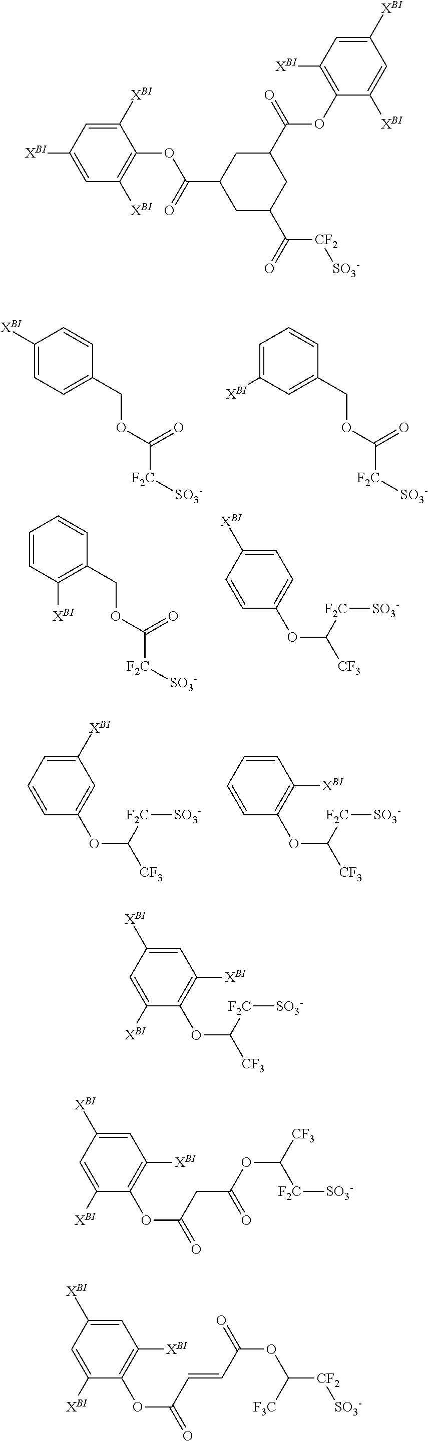

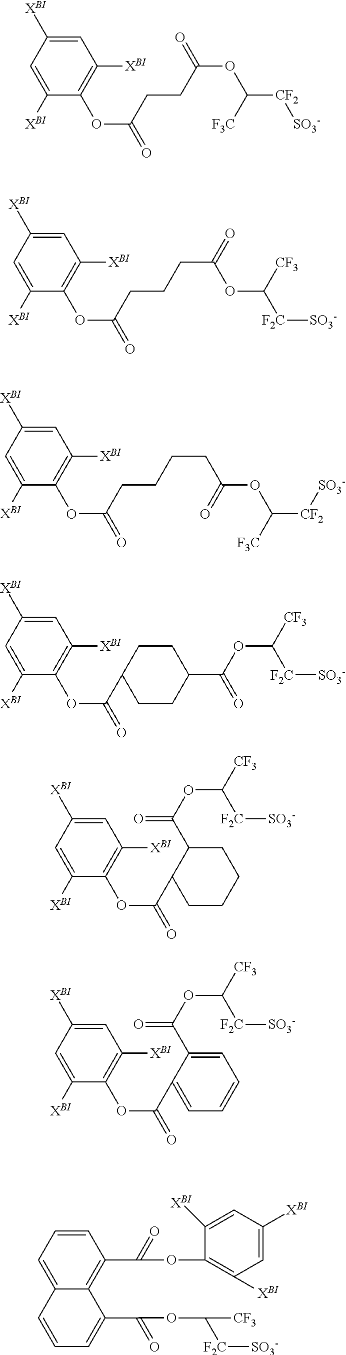

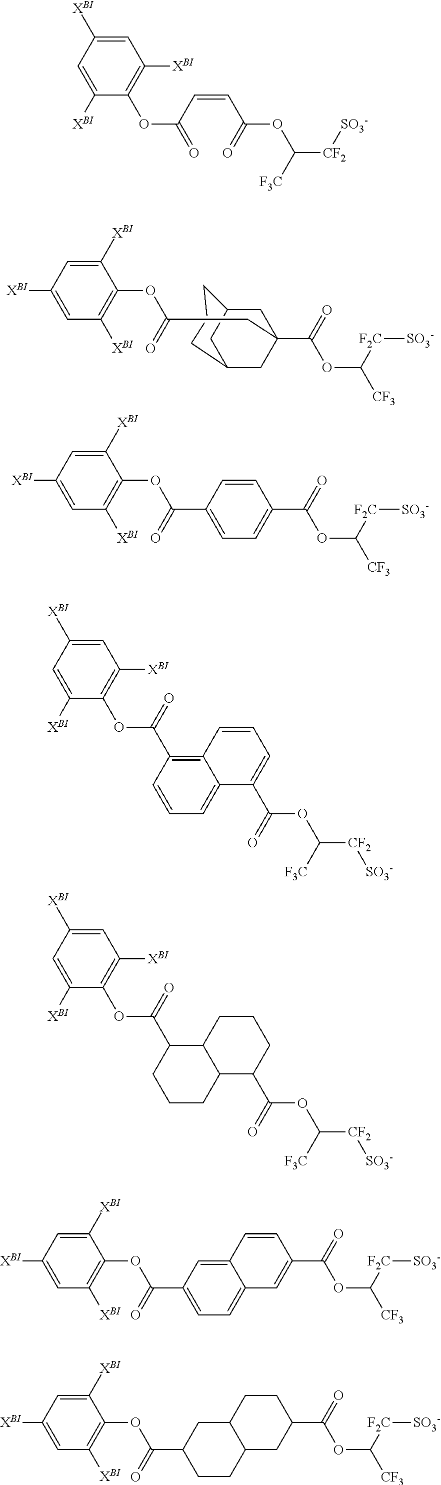

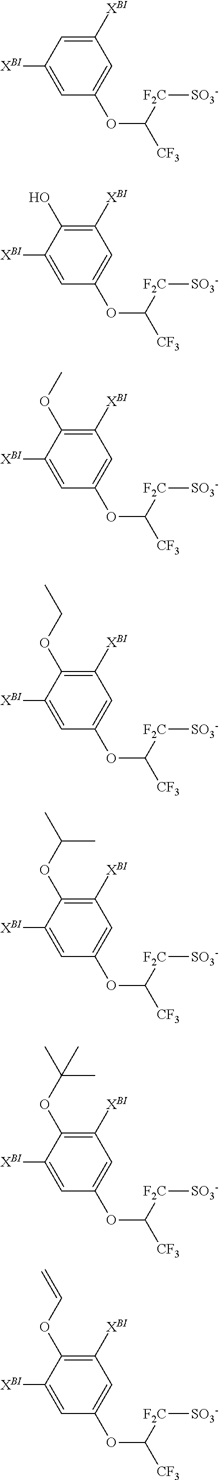

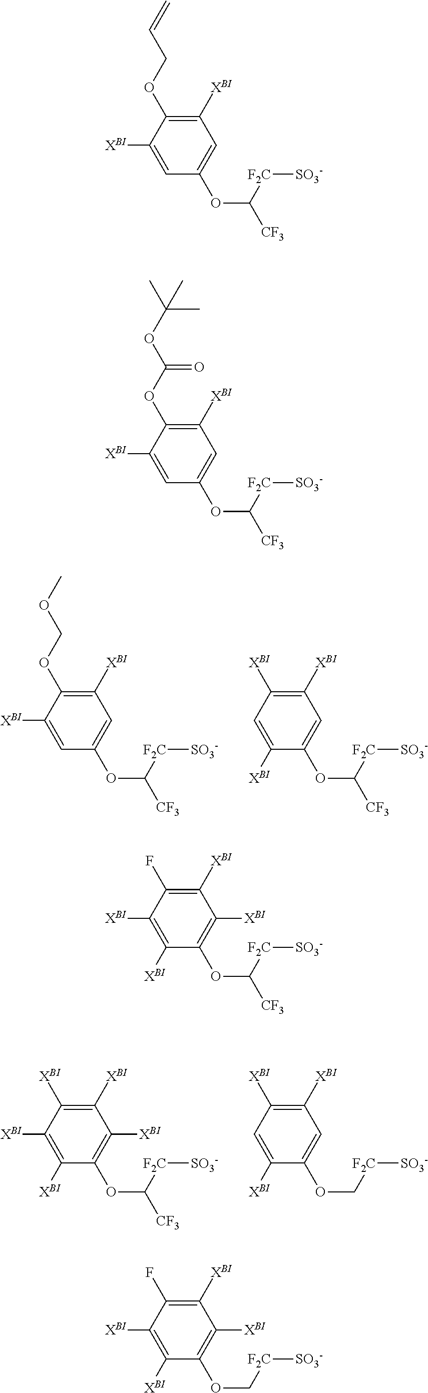

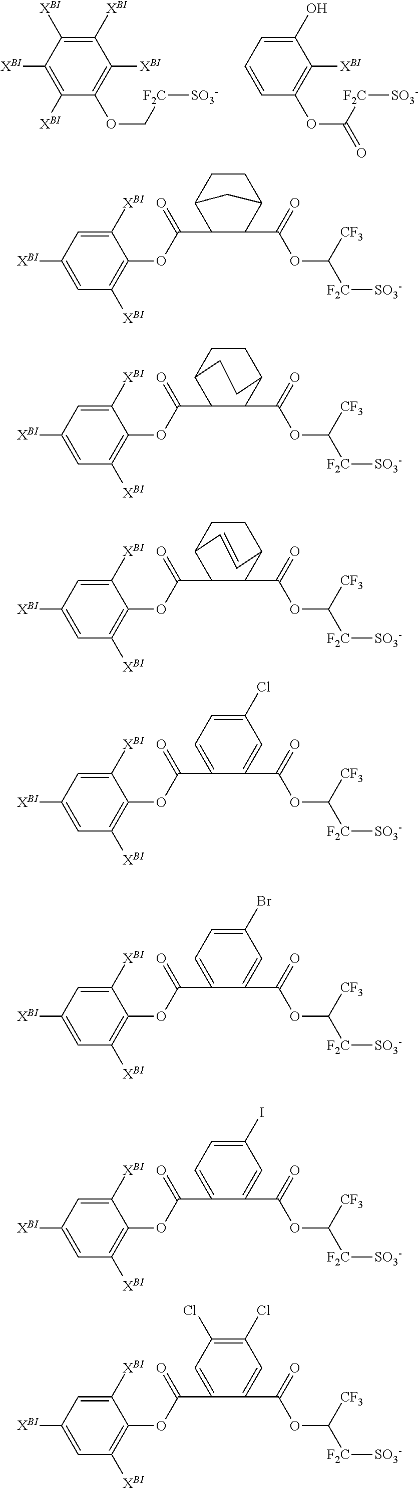

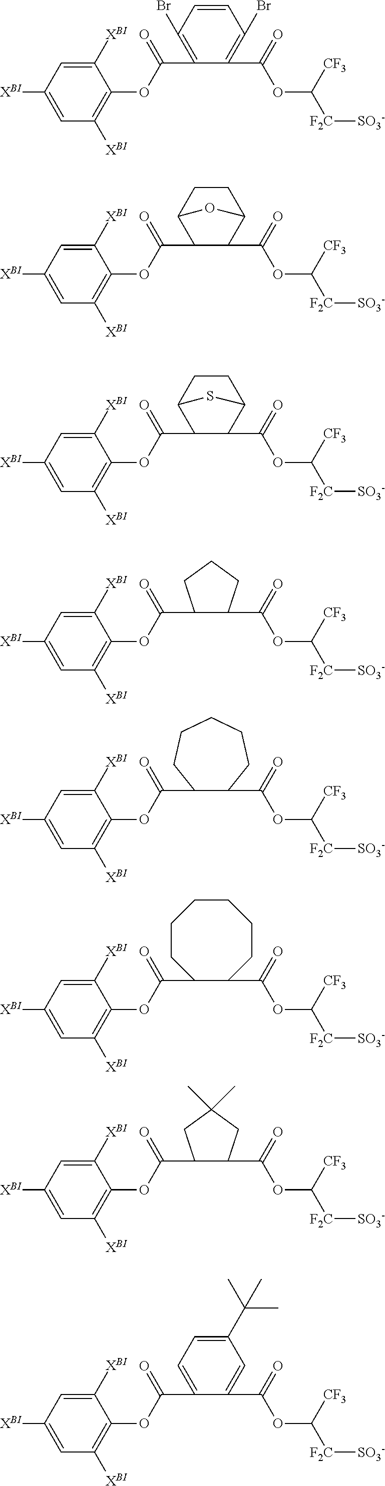

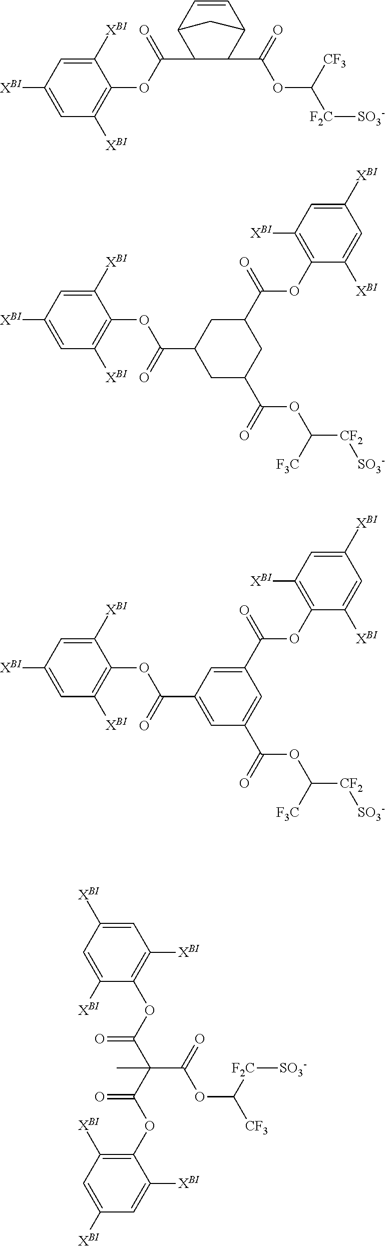

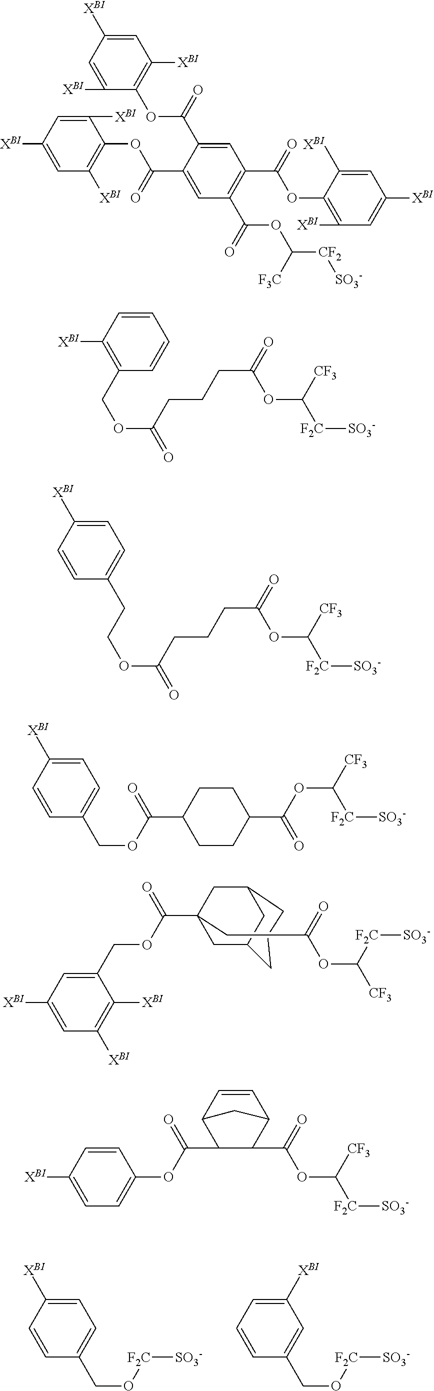

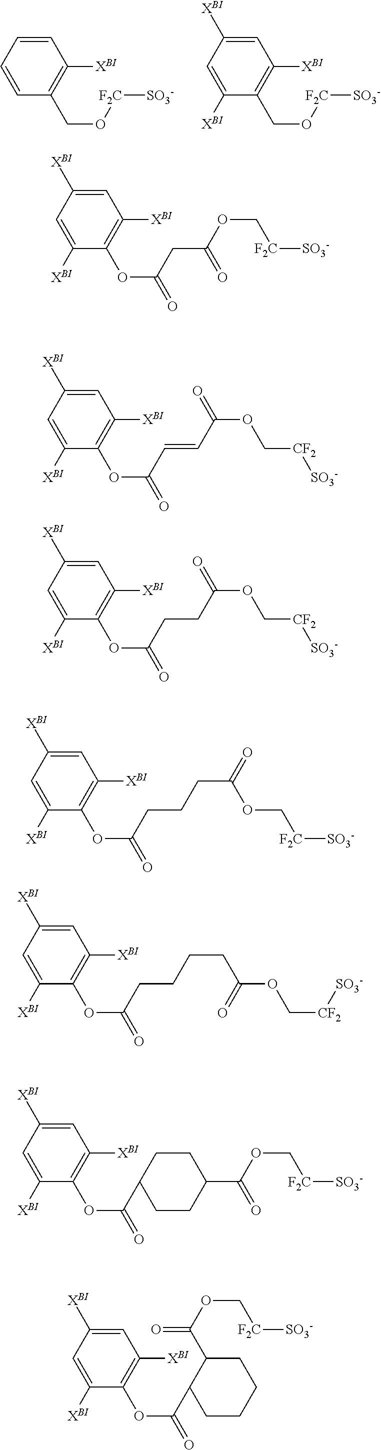

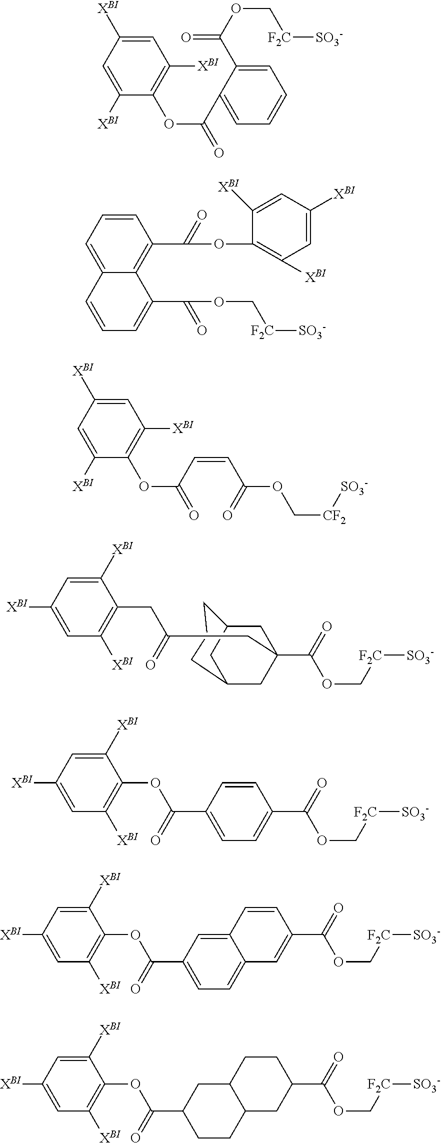

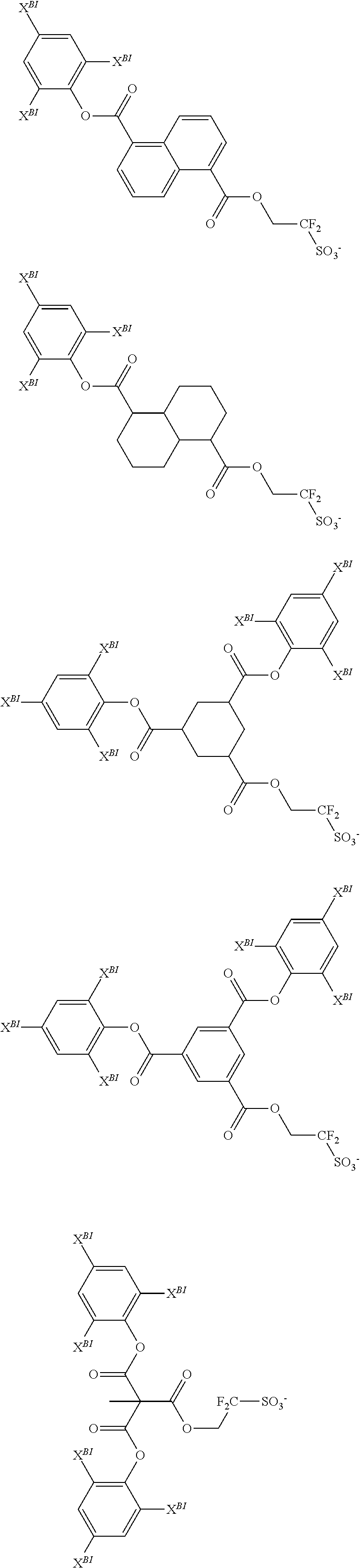

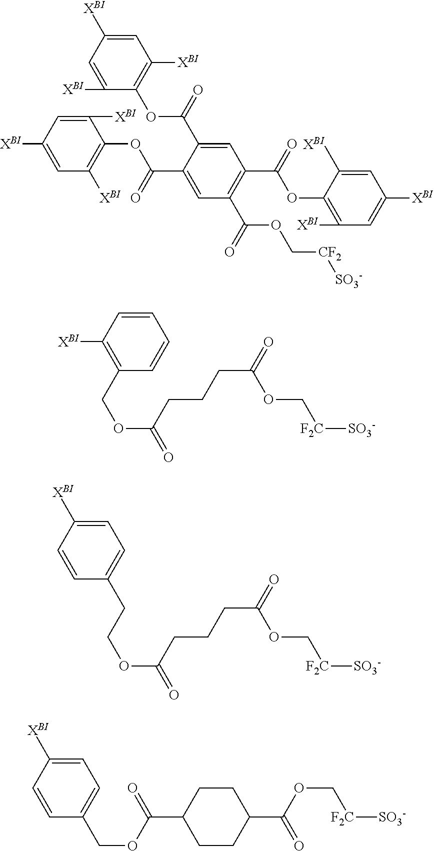

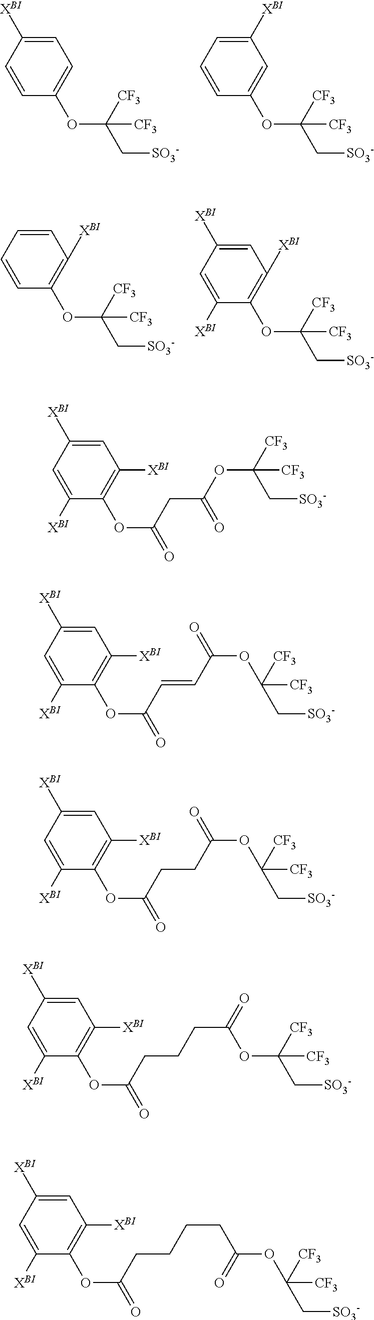

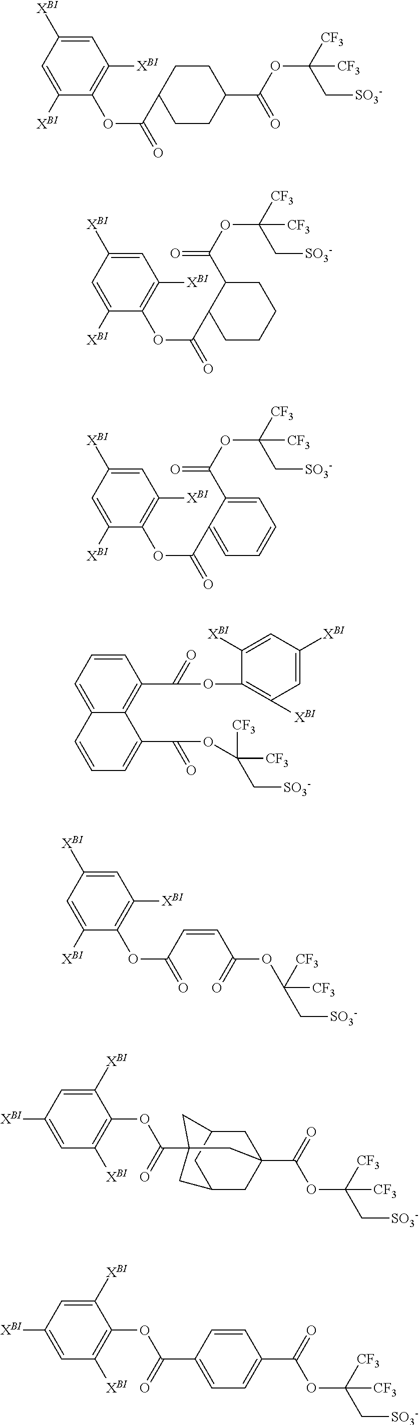

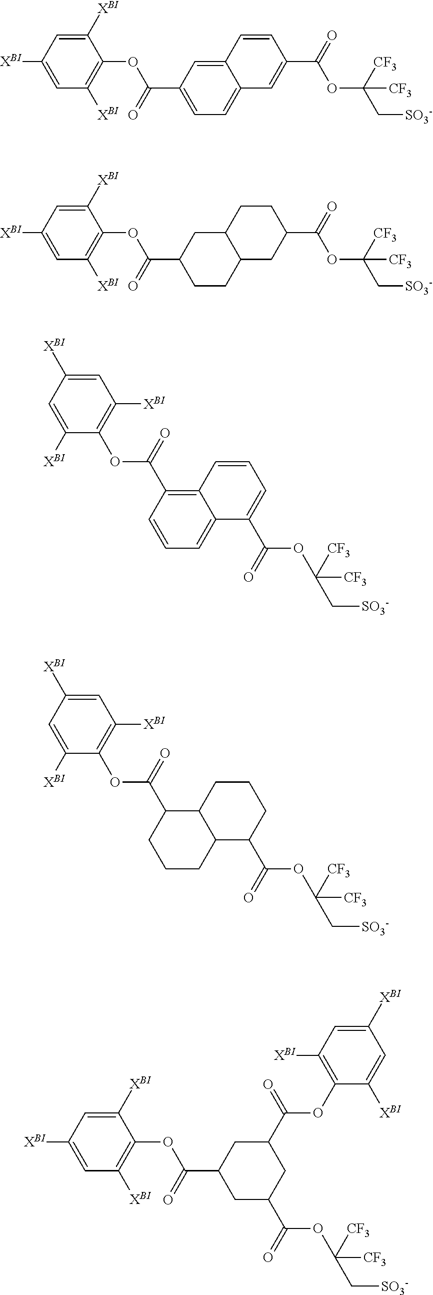

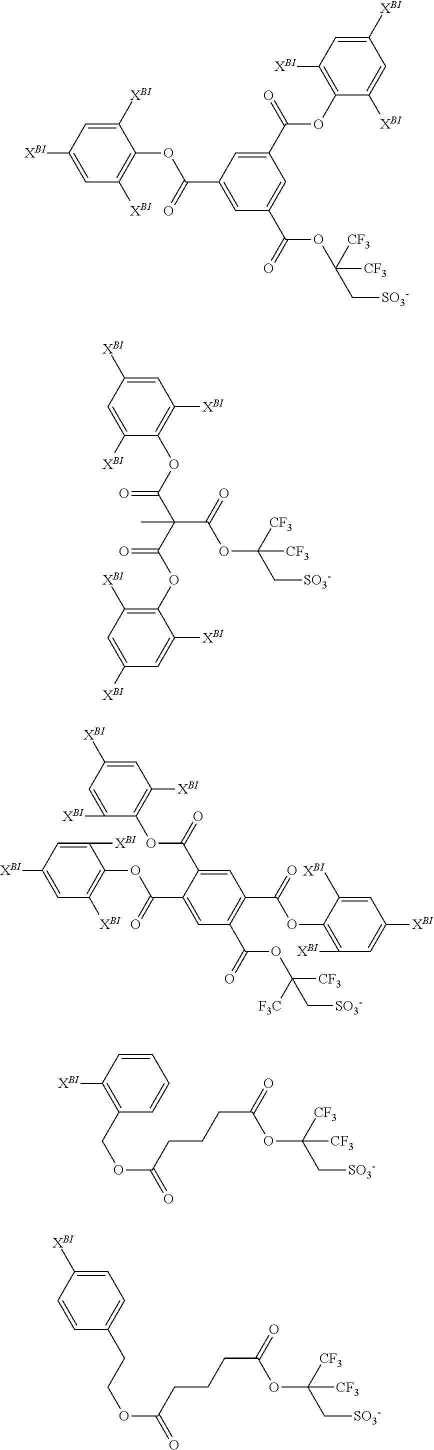

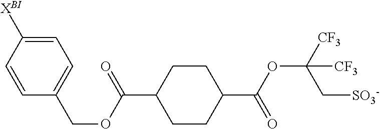

Other useful examples of the non-nucleophilic counter ion include fluorobenzenesulfonic acid anions having an iodized aromatic ring bonded thereto as described in JP 6648726, anions having an acid-catalyzed decomposition mechanism as described in WO 2021/200056 and JP-A 2021-070692, anions having a cyclic ether group as described in JP-A 2018-180525 and JP-A 2021-035935, and anions as described in JP-A 2018-092159.

Further useful examples of the non-nucleophilic counter ion include fluorine-free bulky benzenesulfonic acid anions as described in JP-A 2006-276759, JP-A 2015-117200, JP-A 2016-065016, and JP-A 2019-202974; fluorine-free benzenesulfonic acid or alkylsulfonic acid anions having an iodized aromatic group bonded thereto as described in JP 6645464.

Also useful are bissulfonic acid anions as described in JP-A 2015-206932, sulfonamide or sulfonimide anions having sulfonic acid side and different side as described in WO 2020/158366, and anions having a sulfonic acid side and a carboxylic acid side as described in JP-A 2015-024989.

As PAG (D), a compound having the formula (5) is preferred.

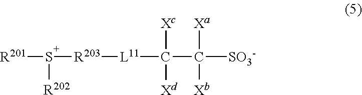

In formula (5), R²⁰¹ and R²⁰² are each independently a C₁-C₃₀ hydrocarbyl group which may contain a heteroatom. R²⁰³ is a C₁-C₃₀ hydrocarbylene group which may contain a heteroatom. Any two of R²⁰¹, R²⁰² and R²⁰³ may bond together to form a ring with the sulfur atom to which they are attached.

The C₁-C₃₀ hydrocarbyl group R²⁰¹ and R²⁰² may be saturated or unsaturated and straight, branched or cyclic. Examples thereof include C₁-C₃₀ alkyl groups such as methyl, ethyl, n-propyl, isopropyl, n-butyl, isobutyl, sec-butyl, tert-butyl, tert-pentyl, n-pentyl, n-hexyl, n-octyl, 2-ethylhexyl, n-nonyl and n-decyl: C₃-C₃₀ cyclic saturated hydrocarbyl groups such as cyclopentyl, cyclohexyl, cyclopentylmethyl, cyclopentylethyl, cyclopentylbutyl, cyclohexylmethyl, cyclohexylethyl, cyclohexylbutyl, norbomnyl, oxanorbornyl, tricyclo[5.2.1.0^{2,6}]decyl, and adamantyl: C₆-C₃₀ aryl groups such as phenyl, methylphenyl, ethylphenyl, n-propylphenyl, isopropylphenyl, n-butylphenyl, isobutylphenyl, sec-butylphenyl, tert-butylphenyl, naphthyl, methylnaphthyl, ethylnaphthyl, n-propylnaphthyl, isopropylnaphthyl, n-butylnaphthyl, isobutylnaphthyl, sec-butylnaphthyl, tert-butylnaphthyl, and anthracenyl; and groups obtained by combining the foregoing. In the hydrocarbyl groups, some or all hydrogen may be substituted by a moiety containing a heteroatom such as oxygen, sulfur, nitrogen or halogen, and some —CH₂— may be replaced by a moiety containing a heteroatom such as oxygen, sulfur or nitrogen, so that the group may contain a hydroxy, cyano, fluorine, chlorine, bromine, iodine, carbonyl, ether bond, ester bond, sulfonate ester bond, carbonate bond, lactone ring, sultone ring, carboxylic anhydride (—C(═O)—O—C(═O)—) or haloalkyl moiety.

The C₁-C₃₀ hydrocarbylene group R²⁰³ may be saturated or unsaturated and straight branched or cyclic. Examples thereof include C₁-C₃₀ alkanediyl groups such as methanediyl, ethane-1,1-diyl, ethane-1,2-diyl, propane-1,3-diyl, butane-1,4-diyl, pentane-1,5-diyl, hexane-1,6-diyl, heptane-1,7-diyl, octane-1,8-diyl, nonane-1,9-diyl, decane-1,10-diyl, undecane-1,11-diyl, dodecane-1,12-diyl, tridecane-1,13-diyl, tetradecane-1,14-diyl, pentadecane-1,15-diyl, hexadecane-1,16-diyl, and heptadecane-1,17-diyl: C₃-C₃₀ cyclic saturated hydrocarbylene groups such as cyclopentanediyl, cyclohexanediyl, norbornanediyl and adamantanediyl; and arylene groups such as phenylene, methylphenylene, ethylphenylene, n-propylphenylene, isopropylphenylene, n-butylphenylene, isobutylphenylene, sec-butylphenylene, tert-butylphenylene, naphthylene, methylnaphthylene, ethylnaphthylene, n-propylnaphthylene, isopropylnaphthylene, n-butylnaphthylene, isobutylnaphthylene, sec-butylnaphthylene, and tert-butylnaphthylene. In these hydrocarbylene groups, some or all of the hydrogen atoms may be substituted by a moiety containing a heteroatom such as oxygen, sulfur, nitrogen or halogen, or some constituent —CH₂— may be replaced by a moiety containing a heteroatom such as oxygen, sulfur or nitrogen, so that the group may contain a hydroxy, cyano, fluorine, chlorine, bromine, iodine, carbonyl, ether bond, ester bond, sulfonate ester bond, carbonate bond, lactone ring, sultone ring, carboxylic anhydride (—C(═O)—O—C(═O)—) or haloalkyl moiety. Of the heteroatoms, oxygen is preferred.

In formula (5), L¹¹ is a single bond, ether bond or a C₁-C₂₀ hydrocarbylene group which may contain a heteroatom. The hydrocarbylene group may be saturated or unsaturated and straight, branched or cyclic. Examples thereof are as exemplified above for the hydrocarbylene group R²⁰³.

In formula (5), X^{a}, X^{b}, X^{c} and X^{d} are each independently hydrogen, fluorine or trifluoromethyl, at least one of X^{a}, X^{b}, X^{c} and X^{d} being fluorine or trifluoromethyl.

Of the PAGs having formula (5), those having formula (5′) are preferred.

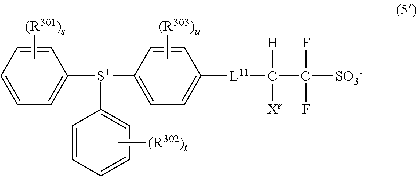

In formula (5′), L¹¹ is as defined above. Xe is hydrogen or trifluoromethyl, preferably trifluoromethyl. R³⁰¹, R³⁰² and R³⁰³ are each independently hydrogen or a C₁-C₂₀ hydrocarbyl group which may contain a heteroatom. The hydrocarbyl group may be saturated or unsaturated and straight, branched or cyclic. Examples thereof are as exemplified above for R^{fa1} in formula (X^{a}-1-1). The subscripts s and t are each independently 0, 1, 2, 3, 4 or 5, and u is 0, 1, 2, 3 or 4.

Examples of the PAG having formula (5) include those exemplified for the PAG having formula (2) in JP-A 2017-026980.

Of the foregoing PAGs, those having an anion of formula (Xa-1-1) or (Xa-4) are especially preferred because of reduced acid diffusion and high solubility in solvents. Also those having formula (5′) are especially preferred because of extremely reduced acid diffusion.

When the resist composition contains the PAG (D), the amount is preferably 0.1 to 40 parts, and more preferably 0.5 to 20 parts by weight per 80 parts by weight of the base polymer (A). As long as the amount of the PAG is in the range, good resolution is achievable and the risk of foreign particles being formed after development or during stripping of resist film is avoided. The PAG may be used alone or in admixture.

### (E) Surfactant

The resist composition may further include (E) a surfactant. Preferred are a surfactant which is insoluble or substantially insoluble in water and soluble in alkaline developer, and a surfactant which is insoluble or substantially insoluble in water and alkaline developer. For the surfactant, reference should be made to those compounds described in JP-A 2010-215608 and JP-A 2011-016746.

While many examples of the surfactant which is insoluble or substantially insoluble in water and alkaline developer are described in the patent documents cited herein, preferred examples are surfactants FC-4430 (3M), Olfine® E1004 (Nissin Chemical Co., Ltd.), Surflon® S-381, KH-20 and KH-30 (AGC Seimi Chemical Co., Ltd.). Partially fluorinated oxetane ring-opened polymers having the formula (surf-1) are also useful.

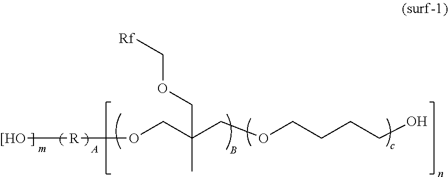

It is provided herein that R, Rf, A, B, C, m, and n are applied to only formula (surf-1), independent of their descriptions other than for the surfactant. R is a di- to tetra-valent C₂-C₅ aliphatic group. Exemplary divalent aliphatic groups include ethylene, 1,4-butylene, 1,2-propylene, 2,2-dimethyl-1,3-propylene and 1,5-pentylene. Exemplary tri- and tetra-valent groups are shown below.

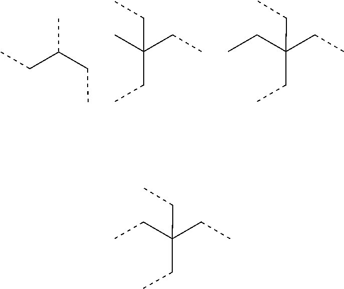

Herein the broken line denotes a valence bond. These formulae are partial structures derived from glycerol, trimethylol ethane, trimethylol propane, and pentaerythritol, respectively. Of these, 1,4-butylene and 2,2-dimethyl-1,3-propylene are preferably used.

Rf is trifluoromethyl or pentafluoroethyl, and preferably trifluoromethyl. The letter m is an integer of 0 to 3, n is an integer of 1 to 4, and the sum of m and n, which represents the valence of R, is an integer of 2 to 4. “A” is equal to 1, B is an integer of 2 to 25, and C is an integer of 0 to 10. Preferably, B is an integer of 4 to 20, and C is 0 or 1. Note that the formula (surf-1) does not prescribe the arrangement of respective constituent units while they may be arranged either blockwise or randomly. For the preparation of surfactants in the form of partially fluorinated oxetane ring-opened polymers, reference should be made to U.S. Pat. No. 5,650,483, for example.

The surfactant which is insoluble or substantially insoluble in water and soluble in alkaline developer is useful when ArF immersion lithography is applied to the resist composition in the absence of a resist protective film. In this embodiment, the surfactant has a propensity to segregate on the resist surface for achieving a function of minimizing water penetration or leaching. The surfactant is also effective for preventing water-soluble components from being leached out of the resist film for minimizing any damage to the exposure tool. The surfactant becomes solubilized during aqueous alkaline development following exposure and PEB, and thus forms few or no foreign particles which become defects. The preferred surfactant is a polymeric surfactant which is insoluble or substantially insoluble in water, but soluble in alkaline developer, also referred to as “hydrophobic resin” in this sense, and especially which is water repellent and enhances water sliding.

Suitable polymeric surfactants include those containing repeat units of at least one type selected from the formulae (6A) to (6E).

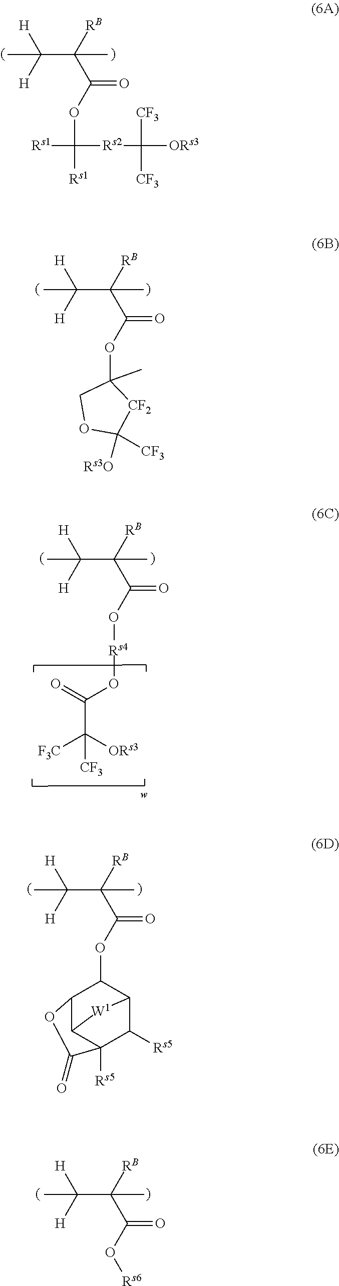

Herein, RB is hydrogen, fluorine, methyl or trifluoromethyl. W¹ is —CH₂—, —CH₂CH₂ or —O—, or two separate —H. R^{s1} is each independently hydrogen or a C₁-C₁₀ hydrocarbyl group. R^{s2} is a single bond or a C₁-C₅ straight or branched hydrocarbylene group. R^{s3} is each independently hydrogen, a C₁-C₁₅ hydrocarbyl or fluorinated hydrocarbyl group, or an acid labile group. When R^{s3} is a hydrocarbyl or fluorinated hydrocarbyl group, an ether bond or carbonyl moiety may intervene in a carbon-carbon bond. R^{s4} is a C₁-C₂₀ (w+1)-valent hydrocarbon or fluorinated hydrocarbon group, and w is 1, 2 or 3. R^{s5} is each independently hydrogen or a group: —C(═O)—O—R^{sa} wherein R^{sa} is a C₁-C₂₀ fluorinated hydrocarbyl group. R^{s6} is a C₁-C₁₅ hydrocarbyl or fluorinated hydrocarbyl group in which an ether bond or carbonyl moiety may intervene in a carbon-carbon bond.

The hydrocarbyl group represented by R^{s1} may be straight, branched or cyclic and is preferably saturated. Examples thereof include C₁-C₁₀ alkyl groups such as methyl, ethyl, n-propyl, isopropyl, n-butyl, isobutyl, sec-butyl, tert-butyl, n-pentyl, n-hexyl, n-heptyl, n-octyl, n-nonyl, n-decyl, and C₃-C₁₀ cyclic saturated hydrocarbyl groups such as cyclopropyl, cyclobutyl, cyclopentyl, cyclohexyl, adamantyl, and norbornyl. Inter alia, C₁-C₆ hydrocarbyl groups are preferred.

The hydrocarbylene group represented by R^{s2} may be straight, branched or cyclic and is preferably saturated. Examples thereof include methylene, ethylene, propylene, butylene and pentylene.

The hydrocarbyl group represented by R^{s3} or R^{s6} may be saturated or unsaturated and straight, branched or cyclic. Examples thereof include saturated hydrocarbyl groups, and aliphatic unsaturated hydrocarbyl groups such as alkenyl and alkynyl groups, with the saturated hydrocarbyl groups being preferred. Suitable saturated hydrocarbyl groups include those exemplified for the hydrocarbyl group represented by R^{s1} as well as undecyl, dodecyl, tridecyl, tetradecyl, and pentadecyl. Examples of the fluorinated hydrocarbyl group represented by R^{s3} or R^{s6} include the foregoing hydrocarbyl groups in which some or all carbon-bonded hydrogen atoms are substituted by fluorine atoms. In these groups, an ether bond or carbonyl moiety may intervene in a carbon-carbon bond as mentioned above.

Examples of the acid labile group represented by R^{s3} include groups of the above formulae (AL-3) to (AL-5), trialkylsilyl groups in which each alkyl moiety has 1 to 6 carbon atoms, and C₄-C₂₀ oxoalkyl groups.

The (w+1)-valent hydrocarbon or fluorinated hydrocarbon group represented by R^{s4} may be straight, branched or cyclic and examples thereof include the foregoing hydrocarbyl or fluorinated hydrocarbyl groups from which “w” number of hydrogen atoms are eliminated.

The fluorinated hydrocarbyl group represented by R^{sa} may be straight, branched or cyclic and is preferably saturated. Examples thereof include the foregoing hydrocarbyl groups in which some or all hydrogen atoms are substituted by fluorine atoms. Illustrative examples include trifluoromethyl, 2,2,2-trifluoroethyl, 3,3,3-trifluoro-1-propyl, 3,3,3-trifluoro-2-propyl, 2,2,3,3-tetrafluoropropyl, 1, 1,1,3,3,3-hexafluoroisopropyl, 2,2,3,3,4,4,4-heptafluorobutyl, 2,2,3,3,4,4,5,5-octafluoropentyl, 2,2,3,3,4,4,5,5,6,6,7,7-dodecafluoroheptyl, 2-(perfluorobutyl)ethyl, 2-(perfluorohexyl)ethyl, 2-(perfluorooctyl)ethyl, and 2-(perfluorodecyl)ethyl.

Examples of the repeat units having formulae (6A) to (6E) are shown below, but not limited thereto. Herein R^{B} is as defined above.

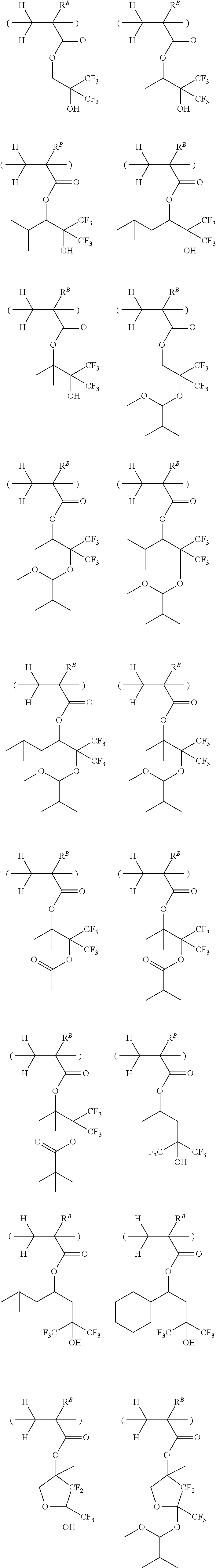

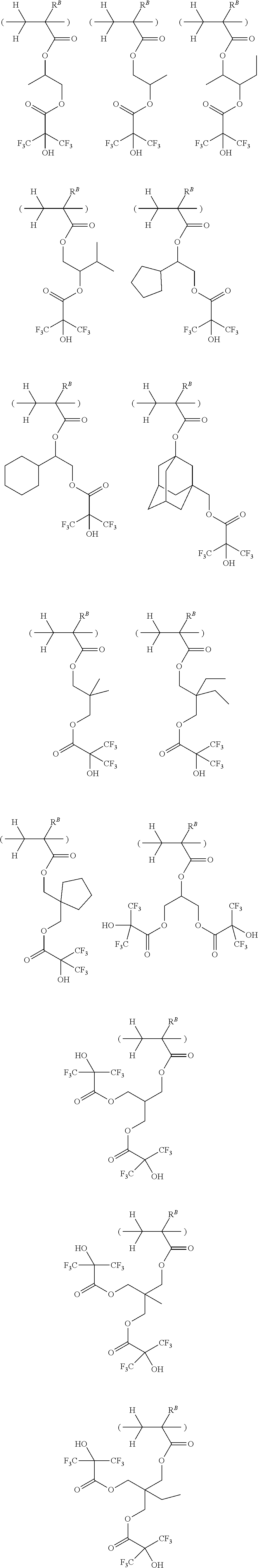

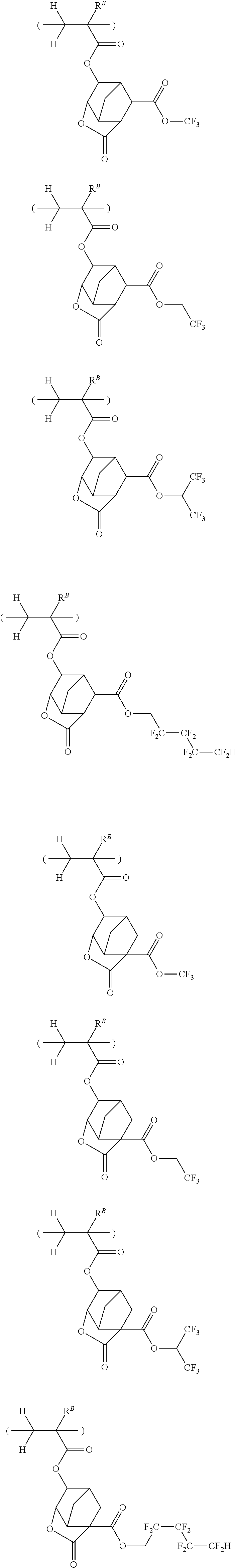

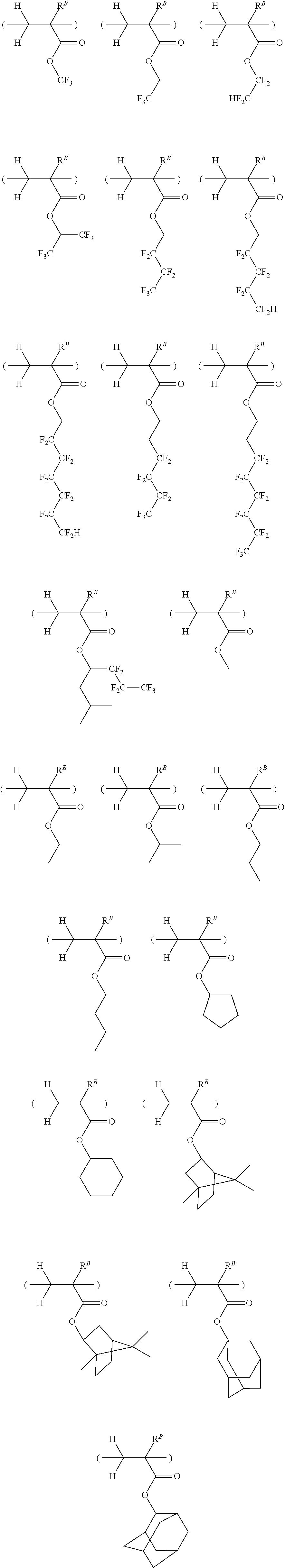

The polymeric surfactant may further contain repeat units other than the repeat units having formulae (6A) to (6E). Typical other repeat units are those derived from methacrylic acid and α-trifluoromethylacrylic acid derivatives. In the polymeric surfactant, the content of the repeat units having formulae (6A) to (6E) is preferably at least 20 mol %, more preferably at least 60 mol %, most preferably 100 mol % of the overall repeat units.

The polymeric surfactant preferably has a Mw of 1,000 to 500,000, more preferably 3,000 to 100,000 and a Mw/Mn of 1.0 to 2.0, more preferably 1.0 to 1.6.

The polymeric surfactant may be synthesized by any desired method, for example, by dissolving an unsaturated bond-containing monomer or monomers providing repeat units having formula (6A) to (6E) and optionally other repeat units in an organic solvent, adding a radical initiator, and heating for polymerization. Suitable organic solvents used herein include toluene, benzene, THF, diethyl ether, and dioxane. Examples of the polymerization initiator used herein include AIBN, 2,2′-azobis(2,4-dimethylvaleronitrile), dimethyl 2,2-azobis(2-methylpropionate), benzoyl peroxide, and lauroyl peroxide. Preferably the reaction temperature is 50 to 100° C. and the reaction time is 4 to 24 hours. The acid labile group that has been incorporated in the monomer may be kept as such, or the polymer may be protected or partially protected therewith at the end of polymerization.

During the synthesis of polymeric surfactant, any known chain transfer agent such as dodecyl mercaptan or 2-mercaptoethanol may be added for molecular weight control purpose. The amount of chain transfer agent added is preferably 0.01 to 10 mol % based on the total moles of monomers to be polymerized.

When the resist composition contains a surfactant (E), the amount thereof is preferably 0.1 to 50 parts by weight, and more preferably 0.5 to 10 parts by weight per 80 parts by weight of the base polymer (A). At least 0.1 part of the surfactant is effective in improving the receding contact angle with water of the resist film at its surface. Up to 50 parts of the surfactant is effective in forming a resist film having a low rate of dissolution in a developer and capable of maintaining the height of a small-size pattern formed therein. The surfactant (E) may be used alone or in admixture.

### (F) Other Components

The resist composition may further comprise (F) another component, for example, a compound which is decomposed with an acid to generate another acid (i.e., acid amplifier compound), an organic acid derivative, a fluorinated alcohol, and a compound having a Mw of up to 3,000 which changes its solubility in developer under the action of an acid (i.e., dissolution inhibitor). Specifically, the acid amplifier compound is described in JP-A 2009-269953 and JP-A 2010-215608 and preferably used in an amount of 0 to 5 parts, more preferably 0 to 3 parts by weight per 80 parts by weight of the base polymer (A). An extra amount of the acid amplifier compound can make the acid diffusion control difficult and cause degradations to resolution and pattern profile. With respect to the remaining additives, reference should be made to JP-A 2009-269953 and JP-A 2010-215608.

### [Process]

A further embodiment of the invention is a process of forming a pattern from the resist composition defined above by lithography. The preferred process includes the steps of applying the resist composition onto a substrate to form a resist film thereon, exposing the resist film to high-energy radiation, and developing the exposed resist film in a developer. Any desired steps may be added to the process if necessary.

The substrate used herein may be a substrate for integrated circuitry fabrication, e.g., Si, SiO₂, SiN, SiON, TiN, WSi, BPSG, SOG, organic antireflective film, etc. or a substrate for mask circuitry fabrication, e.g., Cr, CrO, CrON, MoSi₂, SiO₂, etc.

The resist composition is applied onto a substrate by a suitable coating technique such as spin coating. The coating is prebaked on a hot plate preferably at a temperature of 60 to 150° C. for 1 to 10 minutes, more preferably at 80 to 140° C. for 1 to 5 minutes. The resulting resist film preferably has a thickness of 0.05 to 2 μm.

Then the resist film is exposed to a pattern of high-energy radiation, typically KrF or ArF excimer laser, EUV of wavelength 3 to 15 nm or EB. On use of KrF excimer laser, ArF excimer laser or EUV, the resist film is exposed through a mask having a desired pattern, preferably in a dose of 1 to 200 mJ/cm², more preferably 10 to 100 mJ/cm². On use of EB, a pattern may be written directly or through a mask having the desired pattern, preferably in a dose of 1 to 300 μC/cm², more preferably 10 to 200 μC/cm².

The exposure may be performed by conventional lithography whereas the immersion lithography of holding a liquid having a refractive index of at least 1.0 between the resist film and the projection lens may be employed if desired. The liquid is typically water, and in this case, a protective film which is insoluble in water may be formed on the resist film.

While the water-insoluble protective film serves to prevent any components from being leached out of the resist film and to improve water sliding on the film surface, it is generally divided into two types. The first type is an organic solvent-strippable protective film which must be stripped, prior to alkaline development, with an organic solvent in which the resist film is not dissolvable. The second type is an alkali-soluble protective film which is soluble in an alkaline developer so that it can be removed simultaneously with the removal of solubilized regions of the resist film. The protective film of the second type is preferably of a material comprising a polymer having a 1,1,1,3,3,3-hexafluoro-2-propanol residue (which is insoluble in water and soluble in an alkaline developer) as a base in an alcohol solvent of at least 4 carbon atoms, an ether solvent of 8 to 12 carbon atoms or a mixture thereof. Alternatively, the aforementioned surfactant which is insoluble in water and soluble in an alkaline developer may be dissolved in an alcohol solvent of at least 4 carbon atoms, an ether solvent of 8 to 12 carbon atoms or a mixture thereof to form a material from which the protective film of the second type is formed.

After the exposure, the resist film may be baked (PEB), for example, on a hotplate preferably at 60 to 150° C. for 1 to 5 minutes, more preferably at 80 to 140° C. for 1 to 3 minutes.

The resist film is then developed with a developer in the form of an aqueous base solution, for example, 0.1 to 5 wt %, preferably 2 to 3 wt % aqueous solution of tetramethylammonium hydroxide (TMAH) for 0.1 to 3 minutes, preferably 0.5 to 2 minutes by conventional techniques such as dip, puddle and spray techniques. In this way, the exposed region of the resist film is dissolved away, and a desired resist pattern is formed on the substrate.

Any desired step may be added to the pattern forming process. For example, after the resist film is formed, a step of rinsing with pure water may be introduced to extract the acid generator or the like from the film surface or wash away particles. After exposure, a step of rinsing may be introduced to remove any water remaining on the film after exposure.

Also, a double patterning process may be used for pattern formation. The double patterning process includes a trench process of processing an underlay to a 1:3 trench pattern by a first step of exposure and etching, shifting the position, and forming a 1:3 trench pattern by a second step of exposure, for forming a 1:1 pattern; and a line process of processing a first underlay to a 1:3 isolated left pattern by a first step of exposure and etching, shifting the position, processing a second underlay formed below the first underlay by a second step of exposure through the 1:3 isolated left pattern, for forming a half-pitch 1:1 pattern.

In the pattern forming process, negative tone development may also be used. That is, an organic solvent may be used instead of the aqueous alkaline solution as the developer for developing and dissolving away the unexposed region of the resist film.

The organic solvent used as the developer is preferably selected from 2-octanone, 2-nonanone, 2-heptanone, 3-heptanone, 4-heptanone, 2-hexanone, 3-hexanone, diisobutyl ketone, methylcyclohexanone, acetophenone, methylacetophenone, propyl acetate, butyl acetate, isobutyl acetate, pentyl acetate, isopentyl acetate, butenyl acetate, propyl formate, butyl formate, isobutyl formate, pentyl formate, isopentyl formate, methyl valerate, methyl pentenoate, methyl crotonate, ethyl crotonate, methyl propionate, ethyl propionate, ethyl 3-ethoxypropionate, methyl lactate, ethyl lactate, propyl lactate, butyl lactate, isobutyl lactate, pentyl lactate, isopentyl lactate, methyl 2-hydroxyisobutyrate, ethyl 2-hydroxyisobutyrate, methyl benzoate, ethyl benzoate, phenyl acetate, benzyl acetate, methyl phenylacetate, benzyl formate, phenylethyl formate, methyl 3-phenylpropionate, benzyl propionate, ethyl phenylacetate, and 2-phenylethyl acetate. These organic solvents may be used alone or in admixture of two or more.

### EXAMPLES

Synthesis Examples, Examples and Comparative Examples are given below by way of illustration and not by way of limitation. The abbreviation “pbw” is parts by weight Analysis is made by time-of-flight mass spectrometry using MALDI TOF-MS: S3000 by JEOL Ltd.

### [1] Synthesis of Onium Salt Monomers

### Example 1-1

### Synthesis of Onium Salt Monomer a-1

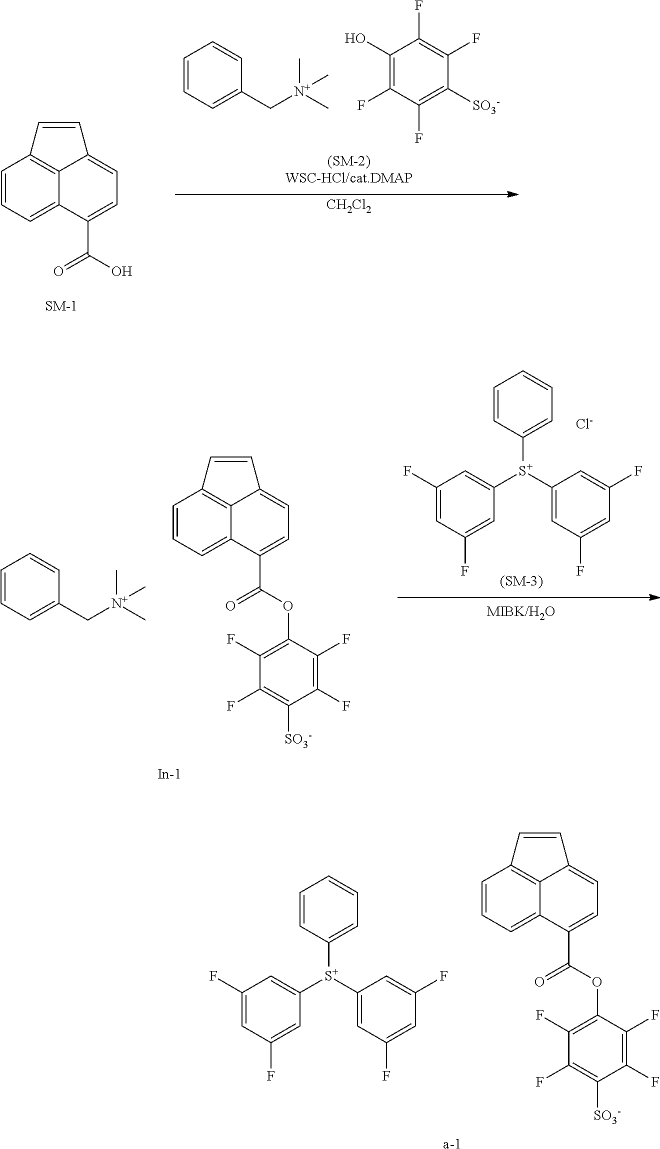

### (1) Synthesis of Intermediate In-1

In nitrogen atmosphere, a reactor was charged with 19.6 g of reactant SM-1, 41.5 g of reactant SM-2, 1.2 g of 4-dimethylaminopyridine, and 200 g of methylene chloride and cooled in an ice bath. While the reactor was kept at a temperature below 20° C., 23.0 g of 1-ethyl-3-(3-dimethylaminopropyl) carbodiimide hydrochloride was added in powder form. The reaction solution was then allowed to warm to room temperature and aged for 12 hours. After aging, 100 g of water was added to quench the reaction. This was followed by ordinary aqueous work-up, solvent distillation, and recrystallization from diisopropyl ether. There was obtained 50.5 g of Intermediate In-1 as white crystals (yield 88%).

### (2) Synthesis of Onium Salt Monomer a-1

In nitrogen atmosphere, a reactor was charged with 50.5 g of Intermediate In-1, 35.9 g of reactant SM-3, 200 g of methylene chloride, and 100 g of water, which were stirred for 30 minutes. The organic layer was taken out, washed with water, and concentrated under reduced pressure. There was obtained 64.8 g of monomer a-1 as oily matter (yield 97%).

Monomer a-1 was analyzed by TOF-MS, with the data shown below.

### MALDI TOF-MS:

positive M⁺ 335 (corresponding to C₁₈H₁₁F₄S⁺)
negative M⁻ 423 (corresponding to C₁₉H₇F₄O₅S⁻)

### Examples 1-2 to 1-7

### Synthesis of Onium Salt Monomers a-2 to a-7

Onium salt monomers a-2 to a-7 shown below were synthesized using corresponding reactants and well-known organic synthesis reactions.

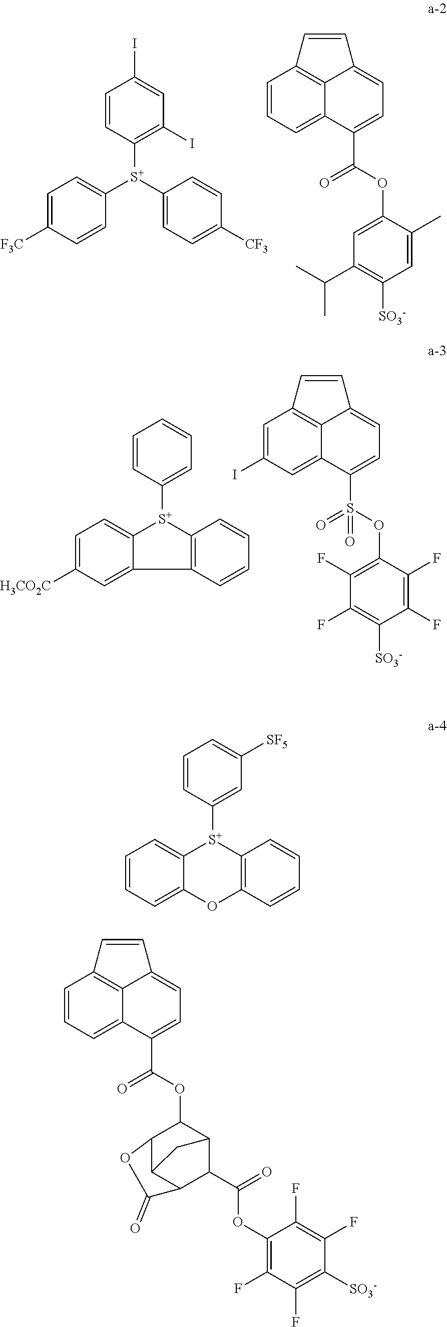

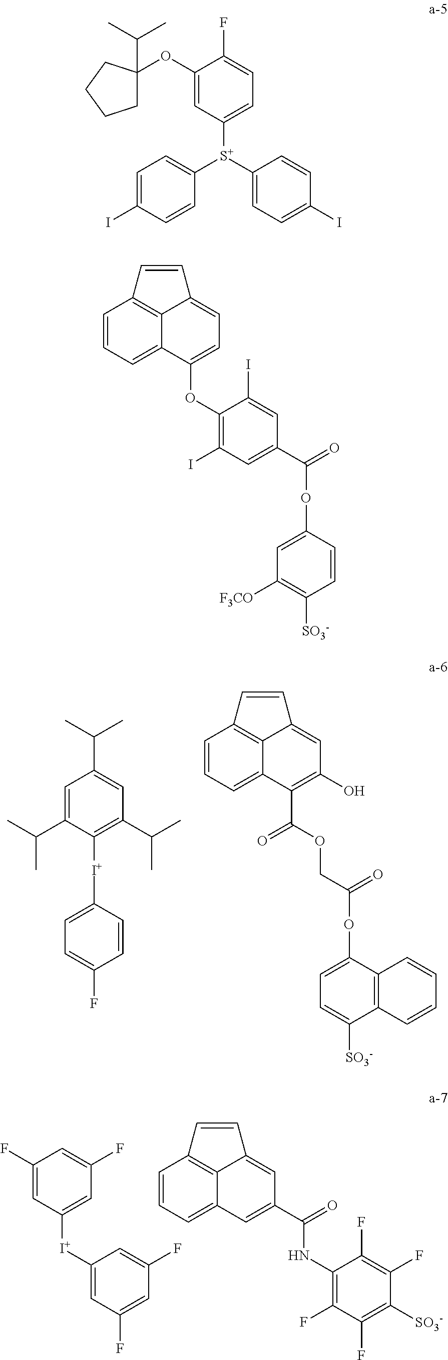

### Comparative Examples 1-1 to 1-4

### Synthesis of Comparative Onium Salt Monomers Ca-1 to Ca-4

Comparative onium salt monomers ca-I to ca-4 shown below were synthesized using corresponding reactants and well-known organic synthesis reactions.

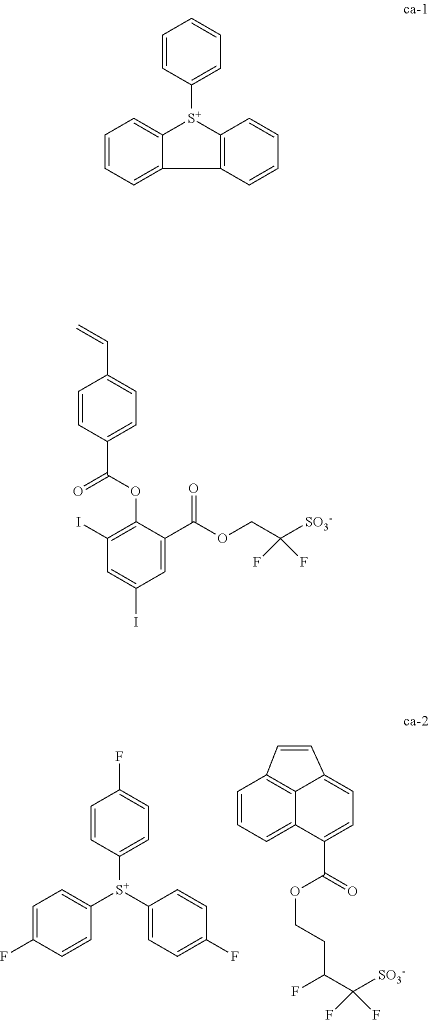

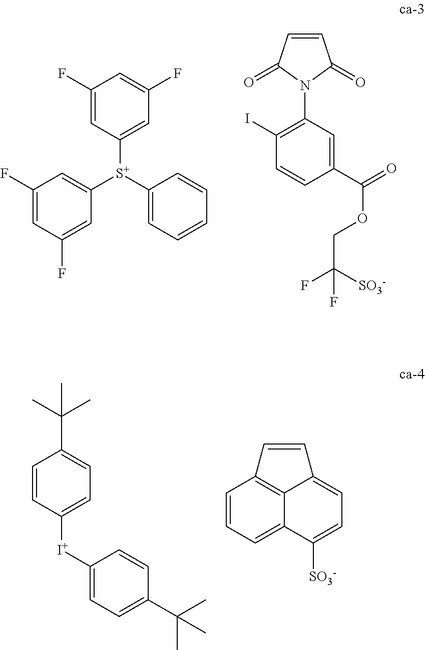

### [2] Synthesis of Base Polymers

In addition to onium salt monomers a-1 to a-7 and comparative onium salt monomers ca-1 to ca-4, the following monomers were used for the synthesis of base polymers.

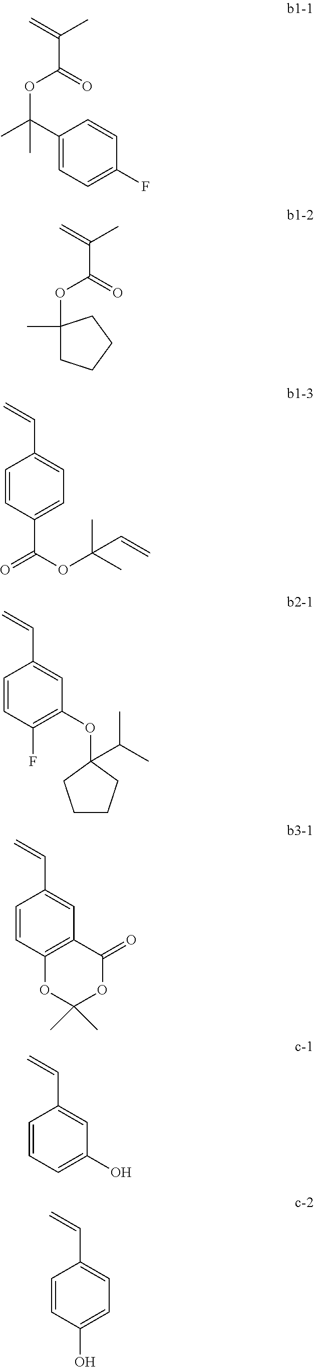

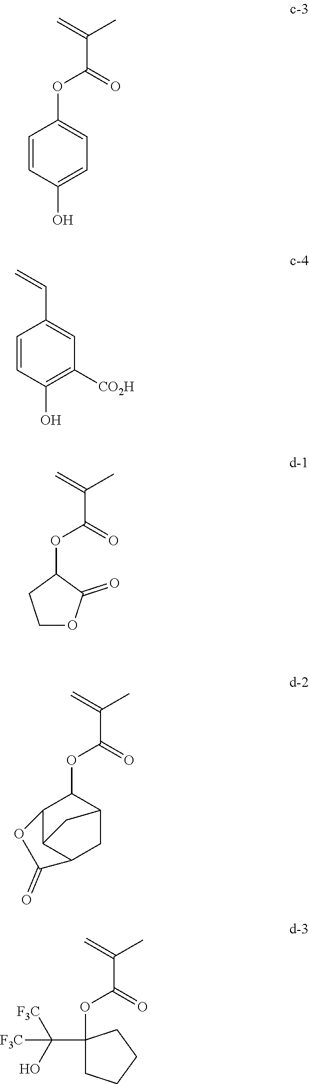

### Example 2-1

### Synthesis of Polymer P-1

A flask under nitrogen atmosphere was charged with 41.8 g of Monomer a-1, 45.1 g of Monomer b1-1, 13.2 g of Monomer c-1, 4.23 g of V-601 (dimethyl 2,2′-azobis(2-methylpropionate) by Fujifilm Wako Pure Chemical Corp.), and 140 g of MEK to form a monomer/initiator solution. Another flask under nitrogen atmosphere was charged with 46 g of MEK, which was heated at 80° C. with stirring. The monomer/initiator solution was added dropwise to the MEK over 4 hours. At the end of addition, the polymerization solution was continuously stirred for 2 hours while maintaining the temperature at 80° C. The polymerization solution was cooled to room temperature, after which it was added dropwise to 3,000 g of hexane with vigorous stirring. The precipitate was collected by filtration. The precipitate was washed twice with 600 g of hexane and vacuum dried at 50° C. for 20 hours, obtaining Polymer P-1 as white powder. Amount 97.3 g, yield 97%. Polymer P-1 had a Mw of 9,900 and a Mw/Mn of 1.55. It is noted that Mw is measured by GPC versus polystyrene standards using DMF solvent.

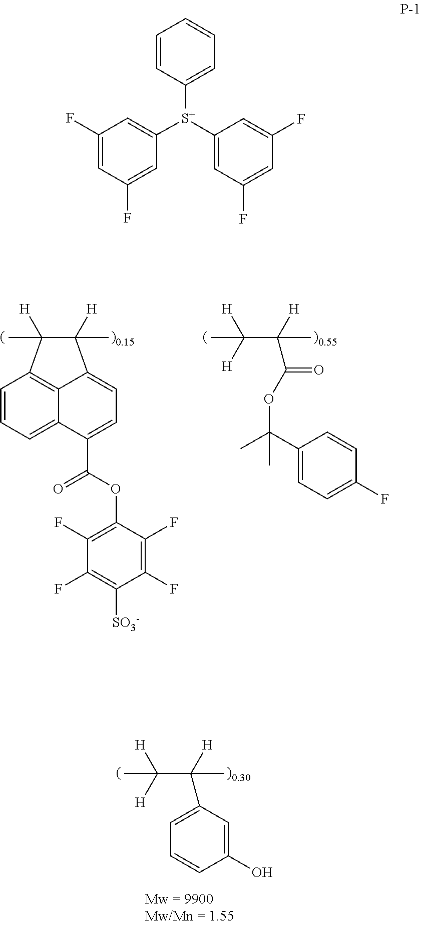

### Examples 2-2 to 2-30 and Comparative Examples 2-1 to 2-24

### Synthesis of Polymers P-2 to P-30 and Comparative Polymers CP-1 to CP-24

Polymers shown in Tables 1 and 2 were synthesized by the same procedure as in Example 2-1 except that the type and amount (blending ratio) of monomers were changed.

TABLE 1

| | | Incorpo- | | Incorpo- | | Incorpo- | | Incorpo- | | Incorpo- | | |
|---|---|---|---|---|---|---|---|---|---|---|---|---|
| | | ration | | ration | | ration | | ration | | ration | | |
| | Unit | ratio | Unit | ratio | Unit | ratio | Unit | ratio | Unit | ratio | | |
| Polymer | 1 | (mol %) | 2 | (mol %) | 3 | (mol %) | 4 | (mol %) | 5 | (mol %) | Mw | Mw/Mn |
| P-1 | a-1 | 15 | b1-1 | 55 | c-1 | 30 | — | — | — | — | 9,900 | 1.55 |
| P-2 | a-2 | 15 | b1-1 | 55 | c-1 | 30 | — | — | — | — | 9,800 | 1.57 |
| P-3 | a-3 | 15 | b1-1 | 55 | c-1 | 30 | — | — | — | — | 9,900 | 1.56 |
| P-4 | a-4 | 15 | b1-1 | 55 | c-1 | 30 | — | — | — | — | 9,700 | 1.55 |
| P-5 | a-5 | 15 | b1-1 | 55 | c-1 | 30 | — | — | — | — | 9,600 | 1.54 |
| P-6 | a-6 | 15 | b1-1 | 55 | c-1 | 30 | — | — | — | — | 9,800 | 1.58 |
| P-7 | a-7 | 15 | b1-1 | 55 | c-1 | 30 | — | — | — | — | 9,900 | 1.56 |
| P-8 | a-1 | 15 | b1-1 | 55 | c-2 | 30 | — | — | — | — | 9,700 | 1.55 |
| P-9 | a-2 | 15 | b1-1 | 55 | c-2 | 30 | — | — | — | — | 9,500 | 1.56 |
| P-10 | a-3 | 15 | b1-1 | 55 | c-2 | 30 | — | — | — | — | 9,800 | 1.55 |
| P-11 | a-4 | 15 | b1-1 | 55 | c-2 | 30 | — | — | — | — | 9,400 | 1.57 |
| P-12 | a-5 | 15 | b1-1 | 55 | c-2 | 30 | — | — | — | — | 9,500 | 1.54 |
| P-13 | a-6 | 15 | b1-1 | 55 | c-2 | 30 | — | — | — | — | 9,700 | 1.55 |
| P-14 | a-7 | 15 | b1-1 | 55 | c-2 | 30 | — | — | — | — | 9,600 | 1.56 |
| P-15 | a-1 | 15 | b1-2 | 55 | c-1 | 30 | — | — | — | — | 9,400 | 1.54 |
| P-16 | a-1 | 15 | b1-3 | 55 | c-1 | 30 | — | — | — | — | 9,500 | 1.57 |
| P-17 | a-1 | 15 | b2-1 | 55 | c-1 | 30 | — | — | — | — | 9,700 | 1.56 |
| P-18 | a-1 | 15 | b3-1 | 45 | c-1 | 40 | — | — | — | — | 9,800 | 1.54 |
| P-19 | a-1 | 15 | b1-1 | 55 | c-3 | 30 | — | — | — | — | 9,900 | 1.57 |
| P-20 | a-1 | 15 | b1-1 | 55 | c-4 | 30 | — | — | — | — | 9,600 | 1.56 |
| P-21 | a-1 | 15 | b1-1 | 30 | b2-1 | 20 | c-1 | 35 | — | — | 9,500 | 1.55 |
| P-22 | a-2 | 15 | b1-1 | 35 | b3-1 | 15 | c-2 | 35 | — | — | 9,600 | 1.57 |
| P-23 | a-3 | 15 | b1-2 | 30 | b2-1 | 15 | c-3 | 40 | — | — | 9,900 | 1.54 |
| P-24 | a-4 | 10 | b1-1 | 35 | b2-1 | 15 | c-1 | 30 | d-1 | 10 | 9,700 | 1.56 |
| P-25 | a-5 | 15 | b1-2 | 35 | b3-1 | 15 | c-2 | 25 | d-2 | 10 | 9,600 | 1.54 |
| P-26 | a-6 | 15 | b1-1 | 50 | c-1 | 30 | d-3 | 5 | — | — | 9,500 | 1.57 |
| P-27 | a-1 | 5 | b1-1 | 50 | c-2 | 40 | — | — | — | — | 9,800 | 1.55 |
| P-28 | a-3 | 5 | b1-1 | 30 | b1-3 | 25 | c-2 | 40 | — | — | 9,700 | 1.56 |
| P-29 | a-5 | 5 | b1-2 | 30 | b2-1 | 20 | c-4 | 35 | d-1 | 10 | 9,800 | 1.55 |
| P-30 | a-7 | 5 | b1-1 | 35 | b3-1 | 15 | c-1 | 30 | d-2 | 15 | 9,700 | 1.57 |

TABLE 2

| | | Incorpo- | | Incorpo- | | Incorpo- | | Incorpo- | | Incorpo- | | |
|---|---|---|---|---|---|---|---|---|---|---|---|---|
| | | ration | | ration | | ration | | ration | | ration | | |
| | Unit | ratio | Unit | ratio | Unit | ratio | Unit | ratio | Unit | ratio | | |
| Polymer | 1 | (mol %) | 2 | (mol %) | 3 | (mol %) | 4 | (mol %) | 5 | (mol %) | Mw | Mw/Mn |
| CP-1 | ca-1 | 15 | b1-1 | 55 | c-1 | 30 | — | — | — | — | 9,600 | 1.54 |
| CP-2 | ca-2 | 15 | b1-1 | 55 | c-1 | 30 | — | — | — | — | 9,400 | 1.57 |
| CP-3 | ca-3 | 15 | b1-1 | 55 | c-1 | 30 | — | — | — | — | 9,500 | 1.56 |
| CP-4 | ca-4 | 15 | b1-1 | 55 | c-1 | 30 | — | — | — | — | 9,700 | 1.54 |
| CP-5 | ca-1 | 15 | b1-1 | 55 | c-2 | 30 | — | — | — | — | 9,800 | 1.57 |
| CP-6 | ca-1 | 15 | b1-1 | 55 | c-2 | 30 | — | — | — | — | 9,900 | 1.56 |
| CP-7 | ca-1 | 15 | b1-1 | 55 | c-2 | 30 | — | — | — | — | 9,600 | 1.55 |
| CP-8 | ca-1 | 15 | b1-1 | 55 | c-2 | 30 | — | — | — | — | 9,500 | 1.57 |
| CP-9 | ca-1 | 15 | b1-2 | 55 | c-1 | 30 | — | — | — | — | 9,600 | 1.54 |
| CP-10 | ca-2 | 15 | b1-3 | 55 | c-1 | 30 | — | — | — | — | 9,500 | 1.56 |
| CP-11 | ca-3 | 15 | b2-1 | 55 | c-1 | 30 | — | — | — | — | 9,800 | 1.54 |
| CP-12 | ca-1 | 15 | b3-1 | 45 | c-1 | 40 | — | — | — | — | 9,400 | 1.57 |
| CP-13 | ca-2 | 15 | b1-1 | 55 | c-3 | 30 | — | — | — | — | 9,500 | 1.56 |
| CP-14 | ca-3 | 15 | b1-1 | 55 | c-4 | 30 | — | — | — | — | 9,600 | 1.55 |
| CP-15 | ca-1 | 15 | b1-1 | 30 | b2-1 | 20 | c-1 | 35 | — | — | 9,500 | 1.57 |
| CP-16 | ca-4 | 15 | b1-1 | 35 | b3-1 | 15 | c-2 | 35 | — | — | 9,600 | 1.54 |
| CP-17 | ca-1 | 15 | b1-2 | 30 | b2-1 | 15 | c-3 | 40 | — | — | 9,900 | 1.56 |
| CP-18 | ca-2 | 10 | b1-1 | 35 | b2-1 | 15 | c-1 | 30 | d-1 | 10 | 9,700 | 1.57 |
| CP-19 | ca-3 | 15 | b1-2 | 35 | b3-1 | 15 | c-2 | 25 | d-2 | 10 | 9,600 | 1.54 |
| CP-20 | ca-3 | 15 | b1-1 | 50 | c-1 | 30 | d-3 | 5 | — | — | 9,500 | 1.56 |
| CP-21 | ca-1 | 5 | b1-1 | 50 | c-2 | 40 | — | — | — | — | 9,800 | 1.55 |
| CP-22 | ca-1 | 5 | b1-1 | 30 | b1-3 | 25 | c-2 | 40 | — | — | 9,800 | 1.56 |
| CP-23 | ca-2 | 5 | b1-2 | 30 | b2-1 | 20 | c-4 | 35 | d-1 | 10 | 9,400 | 1.55 |
| CP-24 | ca-3 | 5 | b1-1 | 35 | b3-1 | 15 | c-1 | 30 | d-2 | 15 | 9,500 | 1.57 |

### [3] Preparation of Resist Composition

### Examples 3-1 to 3-30 and Comparative Examples 3-1 to 3-24

A chemically amplified resist composition (R-1 to R-30, CR-1 to CR-24) was prepared by dissolving a base polymer (P-1 to P-30) or comparative base polymer (CP-1 to CP-24), photoacid generator (PAG-X, PAG-Y), and quencher (Q-1 to Q-4) in an organic solvent containing 0.01 wt % of surfactant FC-4430 (3M) in accordance with the formulation shown in Tables 3 and 4, and filtering the solution through a Teflon® filter with a pore size of 0.2 μm.

TABLE 3

| | | | Base | | Photoacid | | | |
|---|---|---|---|---|---|---|---|---|
| | | Resist | polymer | Quencher | generator | Solvent 1 | Solvent 2 | Solvent 3 |
| | | composition | (pbw) | (pbw) | (pbw) | (pbw) | (pbw) | (pbw) |
| Example | 3-1 | R-1 | P-1 (80) | Q-1 (8.0) | — | PGMEA (2250) | EL (2800) | DAA (550) |
| | 3-2 | R-2 | P-2 (80) | Q-1 (8.2) | — | PGMEA (2250) | EL (2800) | DAA (550) |
| | 3-3 | R-3 | P-3 (80) | Q-1 (7.8) | — | PGMEA (2250) | EL (2800) | DAA (550) |
| | 3-4 | R-4 | P-4 (80) | Q-1 (8.0) | — | PGMEA (2250) | EL (2800) | DAA (550) |
| | 3-5 | R-5 | P-5 (80) | Q-1 (7.6) | — | PGMEA (2250) | EL (2800) | DAA (550) |
| | 3-6 | R-6 | P-6 (80) | Q-1 (8.2) | — | PGMEA (2250) | EL (2800) | DAA (550) |
| | 3-7 | R-7 | P-7 (80) | Q-1 (7.8) | — | PGMEA (2250) | EL (2800) | DAA (550) |
| | 3-8 | R-8 | P-8 (80) | Q-1 (7.6) | — | PGMEA (2250) | EL (2800) | DAA (550) |
| | 3-9 | R-9 | P-9 (80) | Q-1 (8.2) | — | PGMEA (2250) | EL (2800) | DAA (550) |
| | 3-10 | R-10 | P-10 (80) | Q-1 (8.0) | — | PGMEA (2250) | EL (2800) | DAA (550) |
| | 3-11 | R-11 | P-11 (80) | Q-1 (8.2) | — | PGMEA (2250) | EL (2800) | DAA (550) |
| | 3-12 | R-12 | P-12 (80) | Q-1 (7.8) | — | PGMEA (2250) | EL (2800) | DAA (550) |
| | 3-13 | R-13 | P-13 (80) | Q-1 (8.0) | — | PGMEA (2250) | EL (2800) | DAA (550) |
| | 3-14 | R-14 | P-14 (80) | Q-1 (7.8) | — | PGMEA (2250) | EL (2800) | DAA (550) |
| | 3-15 | R-15 | P-15 (80) | Q-1 (8.0) | — | PGMEA (2250) | EL (2800) | DAA (550) |
| | 3-16 | R-16 | P-16 (80) | Q-1 (8.2) | — | PGMEA (2250) | EL (2800) | DAA (550) |
| | 3-17 | R-17 | P-17 (80) | Q-1 (8.0) | — | PGMEA (2250) | EL (2800) | DAA (550) |
| | 3-18 | R-18 | P-18 (80) | Q-1 (7.6) | — | PGMEA (2250) | EL (2800) | DAA (550) |
| | 3-19 | R-19 | P-19 (80) | Q-1 (7.8) | — | PGMEA (2250) | EL (2800) | DAA (550) |
| | 3-20 | R-20 | P-20 (80) | Q-1 (8.2) | — | PGMEA (2250) | EL (2800) | DAA (550) |
| | 3-21 | R-21 | P-21 (80) | Q-2 (8.2) | — | PGMEA (2250) | EL (2800) | DAA (550) |
| | 3-22 | R-22 | P-22 (80) | Q-3 (7.8) | — | PGMEA (2250) | EL (2800) | DAA (550) |
| | 3-23 | R-23 | P-23 (80) | Q-2 (8.2) | — | PGMEA (2250) | EL (2800) | DAA (550) |
| | 3-24 | R-24 | P-24 (80) | Q-1 (8.0) | PAG-X (10) | PGMEA (2250) | EL (2800) | DAA (550) |
| | 3-25 | R-25 | P-25 (80) | Q-1 (4.0) | — | PGMEA (2250) | EL (2800) | DAA (550) |
| | | | | Q-4 (3.6) | | | | |
| | 3-26 | R-26 | P-26 (80) | Q-2 (7.8) | — | PGMEA (2250) | EL (2800) | DAA (550) |
| | 3-27 | R-27 | P-27 (80) | Q-3 (7.8) | — | PGMEA (2250) | EL (2800) | DAA (550) |
| | 3-28 | R-28 | P-28 (80) | Q-1 (8.0) | PAG-Y (13) | PGMEA (2250) | EL (2800) | DAA (550) |
| | 3-29 | R-29 | P-29 (80) | Q-2 (8.2) | PAG-Y (15) | PGMEA (2250) | EL (2800) | DAA (550) |
| | 3-30 | R-30 | P-30 (80) | Q-1 (8.0) | PAG-X (13) | PGMEA (2250) | EL (2800) | DAA (550) |

TABLE 4

| | | | | | Photoacid | | | |
|---|---|---|---|---|---|---|---|---|
| | | Resist | Base polymer | Quencher | generator | Solvent 1 | Solvent 2 | Solvent 3 |
| | | composition | (pbw) | (pbw) | (pbw) | (pbw) | (pbw) | (pbw) |
| Comparative | 3-1 | CR-1 | CP-1 (80) | Q-1 (8.0) | — | PGMEA (2250) | EL (2800) | DAA (550) |
| Example | 3-2 | CR-2 | CP-2 (80) | Q-1 (8.2) | — | PGMEA (2250) | EL (2800) | DAA (550) |
| | 3-3 | CR-3 | CP-3 (80) | Q-1 (7.8) | — | PGMEA (2250) | EL (2800) | DAA (550) |
| | 3-4 | CR-4 | CP-4 (80) | Q-1 (8.0) | — | PGMEA (2250) | EL (2800) | DAA (550) |
| | 3-5 | CR-5 | CP-5 (80) | Q-1 (7.6) | — | PGMEA (2250) | EL (2800) | DAA (550) |
| | 3-6 | CR-6 | CP-6 (80) | Q-1 (8.2) | — | PGMEA (2250) | EL (2800) | DAA (550) |
| | 3-7 | CR-7 | CP-7 (80) | Q-1 (7.8) | — | PGMEA (2250) | EL (2800) | DAA (550) |
| | 3-8 | CR-8 | CP-8 (80) | Q-1 (7.6) | — | PGMEA (2250) | EL (2800) | DAA (550) |
| | 3-9 | CR-9 | CP-9 (80) | Q-1 (8.2) | — | PGMEA (2250) | EL (2800) | DAA (550) |
| | 3-10 | CR-10 | CP-10 (80) | Q-1 (8.0) | — | PGMEA (2250) | EL (2800) | DAA (550) |
| | 3-11 | CR-11 | CP-11 (80) | Q-1 (8.2) | — | PGMEA (2250) | EL (2800) | DAA (550) |
| | 3-12 | CR-12 | CP-12 (80) | Q-1 (7.8) | — | PGMEA (2250) | EL (2800) | DAA (550) |
| | 3-13 | CR-13 | CP-13 (80) | Q-1 (8.0) | — | PGMEA (2250) | EL (2800) | DAA (550) |
| | 3-14 | CR-14 | CP-14 (80) | Q-1 (7.8) | — | PGMEA (2250) | EL (2800) | DAA (550) |
| | 3-15 | CR-15 | CP-15 (80) | Q-2 (8.0) | — | PGMEA (2250) | EL (2800) | DAA (550) |
| | 3-16 | CR-16 | CP-16 (80) | Q-3 (7.8) | — | PGMEA (2250) | EL (2800) | DAA (550) |
| | 3-17 | CR-17 | CP-17 (80) | Q-2 (8.0) | — | PGMEA (2250) | EL (2800) | DAA (550) |
| | 3-18 | CR-18 | CP-18 (80) | Q-1 (7.8) | PAG-X (10) | PGMEA (2250) | EL (2800) | DAA (550) |
| | 3-19 | CR-19 | CP-19 (80) | Q-1 (4.0) | — | PGMEA (2250) | EL (2800) | DAA (550) |
| | | | | Q-4 (3.8) | | | | |
| | 3-20 | CR-20 | CP-20 (80) | Q-2 (7.6) | — | PGMEA (2250) | EL (2800) | DAA (550) |
| | 3-21 | CR-21 | CP-21 (80) | Q-3 (8.0) | — | PGMEA (2250) | EL (2800) | DAA (550) |
| | 3-22 | CR-22 | CP-22 (80) | Q-1 (8.2) | PAG-Y (13) | PGMEA (2250) | EL (2800) | DAA (550) |
| | 3-23 | CR-23 | CP-23 (80) | Q-2 (8.0) | PAG-Y (15) | PGMEA (2250) | EL (2800) | DAA (550) |
| | 3-24 | CR-24 | CP-24 (80) | Q-1 (7.8) | PAG-X (13) | PGMEA (2250) | EL (2800) | DAA (550) |

The components in Tables 3 and 4 are identified below.

### Organic Solvent

PGMEA: propylene glycol monomethyl ether acetate
EL: ethyl lactate
DAA: diacetone alcohol

### Photoacid Generators: PAG-X and PAG-Y

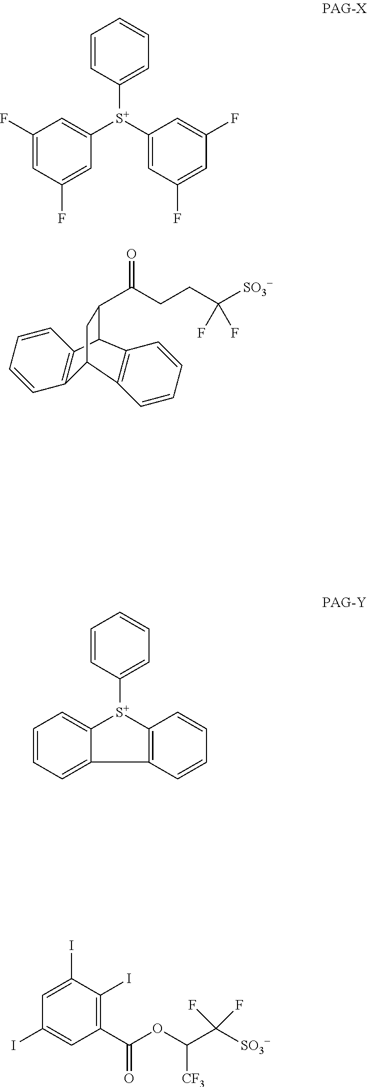

### Quenchers: Q-1 to Q-4

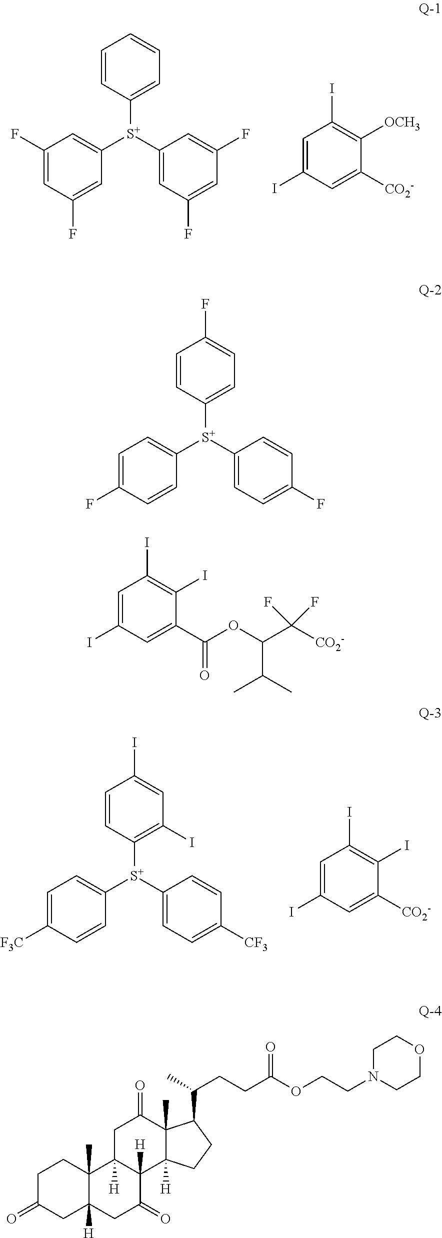

### Surfactant A:

3-methyl-3-(2,2,2-trifluoroethoxymethyl)-oxetane/tetrahydrofuran/2,2-dimethyl-1,3-propane diol copolymer (Omnova Solutions. Inc.)

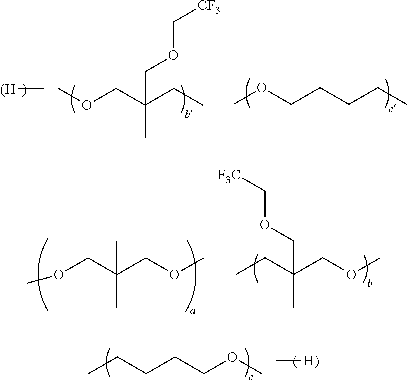

a:(b+b′):(c+c′)=1:4-7:0.01-1 (molar ratio)
Mw=1.500

### [4] EUV Lithography Test 1

### Examples 4-1 to 4-30 and Comparative Examples 4-1 to 4-24

Each of the chemically amplified resist compositions (R-1 to R-30, CR-1 to CR-24 in Tables 3 and 4) was spin coated on a silicon substrate having a 20-nm coating of silicon-containing spin-on hard mask SHB-A940 (Shin-Etsu Chemical Co., Ltd., silicon content 43 wt %) and prebaked on a hotplate at 100° C. for 60 seconds to form a resist film of 50 nm thick. Using an EUV scanner NXE3400 (ASML, NA 0.33, @ 0.9/0.6, dipole illumination), the resist film was exposed to EUV through a mask bearing a line-and-space (LS) pattern having a width of 18 nm and a pitch of 36 nm (on-wafer size) while changing the dose at a pitch of 1 mJ/cm² and the focus at a pitch of 0.020 μm. The resist film was baked (PEB) at the temperature shown in Tables 5 and 6 for 60 seconds. This was followed by puddle development in a 2.38 wt % TMAH aqueous solution for 30 seconds, rinsing with a surfactant-containing rinse fluid, and spin drying. A positive LS pattern was obtained.

The LS pattern was observed under CD-SEM (CG6300, Hitachi High-Technologies Corp.) and evaluated for sensitivity, exposure latitude (EL), LWR, depth of focus (DOF), and collapse limit by the following methods. The results are shown in Tables 5 and 6.

### [Evaluation of Sensitivity]

The optimum dose Eop (mJ/cm²) which provided an LS pattern with a line width of 18 nm and a pitch of 36 nm was determined and reported as sensitivity. A smaller value indicates a higher sensitivity.

### [Evaluation of EL]

The exposure dose which provided a LS pattern with a space width of 18 nm±10% (i.e., 16.2 to 19.8 nm) was determined. EL (%) is calculated from the exposure doses according to the following equation:

$$EL ( \% ) = ( \vert {E}_{1} - {E}_{2} \vert / Eop ) \times 100$$

wherein E1 is an optimum exposure dose which provides a LS pattern with a line width of 16.2 nm and a pitch of 36 nm, E2 is an optimum exposure dose which provides a LS pattern with a line width of 19.8 nm and a pitch of 36 nm, and Eop is an optimum exposure dose which provides a LS pattern with a line width of 18 nm and a pitch of 36 nm. A larger value indicates better performance.

### [Evaluation of LWR]

For the LS pattern formed by exposure at the optimum dose Eop, the line width was measured at 10 longitudinally spaced apart points, from which a 3-fold value (30) of the standard deviation (o) was determined and reported as LWR. A smaller value of 30 indicates a pattern having small roughness and uniform line width.

### [Evaluation of DOF]

As an index of DOF, a range of focus which provided a LS pattern with a size of 18 nm±10% (i.e., 16.2 to 19.8 nm) was determined. A greater value indicates a wider DOF.

### [Evaluation of Collapse Limit of Line Pattern]

For the LS pattern formed by exposure at the dose corresponding to the optimum focus, the line width was measured at 10 longitudinally spaced apart points. The minimum line size above which lines could be resolved without collapse was determined and reported as collapse limit. A smaller value indicates better collapse limit.

TABLE 5

| | | Resist | PEB temp. | Eop | EL | LWR | DOF | Collapse limit |
|---|---|---|---|---|---|---|---|---|
| | | composition | (° C.) | (mJ/cm²) | (%) | (nm) | (nm) | (nm) |
| Example | 4-1 | R-1 | 100 | 33 | 19 | 2.3 | 120 | 10.7 |
| | 4-2 | R-2 | 100 | 34 | 17 | 2.2 | 110 | 10.8 |
| | 4-3 | R-3 | 100 | 32 | 18 | 2.3 | 120 | 11.2 |
| | 4-4 | R-4 | 105 | 33 | 19 | 2.4 | 110 | 10.9 |
| | 4-5 | R-5 | 105 | 34 | 17 | 2.2 | 100 | 11.1 |
| | 4-6 | R-6 | 95 | 33 | 18 | 2.2 | 120 | 10.9 |
| | 4-7 | R-7 | 95 | 34 | 18 | 2.4 | 110 | 11.5 |
| | 4-8 | R-8 | 95 | 34 | 19 | 2.3 | 100 | 11.3 |
| | 4-9 | R-9 | 105 | 33 | 19 | 2.3 | 110 | 11.4 |
| | 4-10 | R-10 | 95 | 32 | 18 | 2.5 | 120 | 10.9 |
| | 4-11 | R-11 | 100 | 34 | 19 | 2.3 | 120 | 11.5 |
| | 4-12 | R-12 | 95 | 34 | 17 | 2.2 | 110 | 11.2 |
| | 4-13 | R-13 | 105 | 33 | 18 | 2.4 | 120 | 11.4 |
| | 4-14 | R-14 | 105 | 35 | 17 | 2.4 | 100 | 11.6 |
| | 4-15 | R-15 | 95 | 34 | 19 | 2.3 | 110 | 11.1 |
| | 4-16 | R-16 | 100 | 33 | 18 | 2.3 | 120 | 10.7 |
| | 4-17 | R-17 | 100 | 34 | 17 | 2.2 | 110 | 10.8 |
| | 4-18 | R-18 | 95 | 32 | 19 | 2.5 | 110 | 11.1 |
| | 4-19 | R-19 | 100 | 34 | 18 | 2.3 | 120 | 11.3 |
| | 4-20 | R-20 | 95 | 35 | 17 | 2.2 | 110 | 11.2 |
| | 4-21 | R-21 | 95 | 34 | 18 | 2.4 | 120 | 10.9 |
| | 4-22 | R-22 | 100 | 33 | 19 | 2.3 | 120 | 11.3 |
| | 4-23 | R-23 | 95 | 33 | 17 | 2.4 | 110 | 11.2 |
| | 4-24 | R-24 | 105 | 32 | 18 | 2.5 | 120 | 11.4 |
| | 4-25 | R-25 | 105 | 34 | 17 | 2.3 | 100 | 11.3 |
| | 4-26 | R-26 | 95 | 33 | 19 | 2.5 | 110 | 11.1 |
| | 4-27 | R-27 | 100 | 35 | 18 | 2.4 | 120 | 10.9 |
| | 4-28 | R-28 | 100 | 34 | 17 | 2.3 | 110 | 11.0 |
| | 4-29 | R-29 | 95 | 32 | 19 | 2.4 | 110 | 11.2 |
| | 4-30 | R-30 | 100 | 33 | 18 | 2.3 | 120 | 11.3 |

TABLE 6

| | | Resist | PEB temp. | Eop | EL | LWR | DOF | Collapse limit |
|---|---|---|---|---|---|---|---|---|
| | | composition | (° C.) | (mJ/cm²) | (%) | (nm) | (nm) | (nm) |
| Comparative | 4-1 | CR-1 | 100 | 35 | 16 | 2.6 | 120 | 12.3 |
| Example | 4-2 | CR-2 | 95 | 35 | 16 | 2.7 | 90 | 11.7 |
| | 4-3 | CR-3 | 105 | 37 | 16 | 2.9 | 90 | 11.5 |
| | 4-4 | CR-4 | 100 | 38 | 15 | 3.1 | 90 | 11.5 |
| | 4-5 | CR-5 | 100 | 35 | 14 | 2.7 | 80 | 12.2 |
| | 4-6 | CR-6 | 95 | 36 | 15 | 2.6 | 100 | 12.3 |
| | 4-7 | CR-7 | 100 | 35 | 15 | 2.8 | 90 | 12.5 |
| | 4-8 | CR-8 | 105 | 36 | 14 | 2.6 | 90 | 12.3 |
| | 4-9 | CR-9 | 95 | 35 | 15 | 2.8 | 80 | 11.9 |
| | 4-10 | CR-10 | 100 | 36 | 15 | 2.7 | 80 | 11.8 |
| | 4-11 | CR-11 | 100 | 37 | 14 | 2.6 | 90 | 12.1 |
| | 4-12 | CR-12 | 100 | 36 | 15 | 2.9 | 90 | 12.3 |
| | 4-13 | CR-13 | 95 | 35 | 14 | 2.7 | 90 | 12.4 |
| | 4-14 | CR-14 | 100 | 37 | 14 | 2.8 | 80 | 11.8 |
| | 4-15 | CR-15 | 105 | 36 | 15 | 2.6 | 100 | 11.9 |
| | 4-16 | CR-16 | 95 | 39 | 14 | 3.1 | 90 | 12.0 |
| | 4-17 | CR-17 | 100 | 35 | 14 | 2.7 | 90 | 11.9 |
| | 4-18 | CR-18 | 100 | 37 | 15 | 2.9 | 80 | 11.8 |
| | 4-19 | CR-19 | 95 | 37 | 15 | 2.8 | 90 | 12.1 |
| | 4-20 | CR-20 | 100 | 36 | 16 | 2.7 | 90 | 12.3 |
| | 4-21 | CR-21 | 105 | 35 | 14 | 2.6 | 80 | 11.7 |
| | 4-22 | CR-22 | 95 | 35 | 15 | 2.9 | 90 | 11.9 |
| | 4-23 | CR-23 | 100 | 36 | 14 | 2.7 | 90 | 11.6 |
| | 4-24 | CR-24 | 95 | 38 | 15 | 2.9 | 80 | 11.8 |

As seen from Tables 5 and 6, chemically amplified resist compositions comprising polymers comprising repeat units derived from onium salt monomers within the scope of the invention exhibit a high sensitivity and improved lithography properties including EL, LWR and DOF. Small values of collapse limit show that small-size patterns have resistance to collapse. It is demonstrated that the chemically amplified resist compositions within the scope of the invention are suited for the EUV lithography process.

### [5] EUV Lithography Test 2

### Examples 5-1 to 5-30 and Comparative Examples 5-1 to 5-24

Each of the chemically amplified resist compositions (R-1 to R-30, CR-1 to CR-24 in Tables 3 and 4) was spin coated on a silicon substrate having a 20-nm coating of silicon-containing spin-on hard mask SHB-A940 (Shin-Etsu Chemical Co., Ltd., silicon content 43 wt %) and prebaked on a hotplate at 105° C. for 60 seconds to form a resist film of 50 nm thick. Using an EUV scanner NXE3400 (ASML, NA 0.33, & 0.9/0.6, quadrupole illumination), the resist film was exposed to EUV through a mask bearing a hole pattern having a pitch of 46 nm+20% bias (on-wafer size). The resist film was baked (PEB) on a hotplate at the temperature shown in Tables 7 and 8 for 60 seconds. This was followed by development in a 2.38 wt % TMAH aqueous solution for 30 seconds. Hole patterns with a size of 23 nm were obtained.

The hole pattern was observed under CD-SEM (CG6300, Hitachi High-Technologies Corp.). The exposure dose Eop that provides a hole pattern having a size of 23 nm was determined and reported as sensitivity. The size of 50 holes at that dose was measured, from which a 3-fold value (3σ) of the standard deviation (σ) was computed and reported as CDU. The results are also shown in Tables 7 and 8.

TABLE 7

| | | Resist | PEB temp. | Eop | CDU |
|---|---|---|---|---|---|
| | | composition | (° C.) | (mJ/cm²) | (nm) |
| Example | 5-1 | R-1 | 90 | 22 | 2.2 |
| | 5-2 | R-2 | 95 | 23 | 2.3 |
| | 5-3 | R-3 | 95 | 25 | 2.6 |
| | 5-4 | R-4 | 95 | 23 | 2.4 |
| | 5-5 | R-5 | 90 | 22 | 2.5 |
| | 5-6 | R-6 | 95 | 23 | 2.5 |
| | 5-7 | R-7 | 100 | 24 | 2.6 |
| | 5-8 | R-8 | 95 | 23 | 2.5 |
| | 5-9 | R-9 | 95 | 24 | 2.3 |
| | 5-10 | R-10 | 100 | 22 | 2.4 |
| | 5-11 | R-11 | 95 | 24 | 2.5 |
| | 5-12 | R-12 | 95 | 24 | 2.5 |
| | 5-13 | R-13 | 90 | 25 | 2.3 |
| | 5-14 | R-14 | 95 | 24 | 2.4 |
| | 5-15 | R-15 | 90 | 23 | 2.4 |
| | 5-16 | R-16 | 85 | 24 | 2.5 |
| | 5-17 | R-17 | 95 | 24 | 2.5 |
| | 5-18 | R-18 | 100 | 23 | 2.4 |
| | 5-19 | R-19 | 95 | 24 | 2.6 |
| | 5-20 | R-20 | 95 | 22 | 2.5 |
| | 5-21 | R-21 | 95 | 24 | 2.5 |
| | 5-22 | R-22 | 95 | 23 | 2.5 |
| | 5-23 | R-23 | 90 | 25 | 2.3 |
| | 5-24 | R-24 | 95 | 24 | 2.4 |
| | 5-25 | R-25 | 90 | 24 | 2.4 |
| | 5-26 | R-26 | 85 | 23 | 2.5 |
| | 5-27 | R-27 | 95 | 25 | 2.5 |
| | 5-28 | R-28 | 100 | 24 | 2.4 |
| | 5-29 | R-29 | 95 | 23 | 2.6 |
| | 5-30 | R-30 | 95 | 24 | 2.5 |

TABLE 8

| | | Resist | PEB temp. | Eop | CDU |
|---|---|---|---|---|---|
| | | composition | (° C.) | (mJ/cm²) | (nm) |
| Comparative | 5-1 | CR-1 | 90 | 26 | 2.6 |
| Example | 5-2 | CR-2 | 95 | 25 | 2.7 |
| | 5-3 | CR-3 | 90 | 27 | 2.8 |
| | 5-4 | CR-4 | 95 | 30 | 3.3 |
| | 5-5 | CR-5 | 95 | 26 | 2.6 |
| | 5-6 | CR-6 | 100 | 27 | 2.5 |
| | 5-7 | CR-7 | 95 | 26 | 2.7 |
| | 5-8 | CR-8 | 95 | 27 | 2.8 |
| | 5-9 | CR-9 | 90 | 26 | 2.7 |
| | 5-10 | CR-10 | 100 | 25 | 2.7 |
| | 5-11 | CR-11 | 90 | 26 | 2.8 |
| | 5-12 | CR-12 | 95 | 27 | 2.9 |
| | 5-13 | CR-13 | 95 | 27 | 2.8 |
| | 5-14 | CR-14 | 100 | 26 | 2.9 |
| | 5-15 | CR-15 | 95 | 25 | 2.7 |
| | 5-16 | CR-16 | 95 | 31 | 3.2 |
| | 5-17 | CR-17 | 90 | 28 | 2.8 |
| | 5-18 | CR-18 | 100 | 27 | 2.9 |
| | 5-19 | CR-19 | 100 | 26 | 2.9 |
| | 5-20 | CR-20 | 95 | 26 | 2.8 |
| | 5-21 | CR-21 | 95 | 27 | 2.9 |
| | 5-22 | CR-22 | 90 | 25 | 2.6 |
| | 5-23 | CR-23 | 95 | 27 | 2.9 |
| | 5-24 | CR-24 | 100 | 28 | 2.8 |

It is demonstrated in Tables 7 and 8 that chemically amplified resist compositions comprising polymers comprising repeat units derived from sulfonium salt monomers within the scope of the invention exhibit a high sensitivity and improved CDU.

### [6] Dry Etching Test

### Examples 6-1 to 6-30 and Comparative Examples 6-1 to 6-24

Each of the polymers (Polymers P-1 to P-30, Comparative Polymers CP-1 to CP-24 in Tables 1 and 2), 2 g, was thoroughly dissolved in 10 g of cyclohexanone, and passed through a filter having a pore size of 0.2 μm, obtaining a polymer solution. The polymer solution was spin coated onto a silicon substrate and baked to form a polymer film of 300 nm thick. Using a dry etching instrument TE-8500P (Tokyo Electron Ltd.), the polymer film was etched with CHF₃/CF₄ gas under the following conditions.

| | | |
|---|---|---|
| Chamber pressure | 40 | Pa |
| RF power | 1000 | W |
| Gap | 9 | mm |
| CHF₃ gas flow rate | 30 | ml/min |
| CF₄ gas flow rate | 30 | ml/min |
| Ar gas flow rate | 100 | ml/min |
| Time | 60 | sec |

The difference in polymer film thickness before and after etching was determined, from which an etching rate per minute was computed. The results are shown in Tables 9 and 10. A smaller value of film thickness difference, i.e., a lower etching rate indicates better etch resistance.

TABLE 9

| | | Polymer | CHF₃/CF₄ gas etching rate (nm/min) |
|---|---|---|---|
| Example | 6-1 | P-1 | 94 |
| | 6-2 | P-2 | 95 |
| | 6-3 | P-3 | 96 |
| | 6-4 | P-4 | 95 |
| | 6-5 | P-5 | 96 |
| | 6-6 | P-6 | 96 |
| | 6-7 | P-7 | 94 |
| | 6-8 | P-8 | 96 |
| | 6-9 | P-9 | 95 |
| | 6-10 | P-10 | 95 |
| | 6-11 | P-11 | 94 |
| | 6-12 | P-12 | 95 |
| | 6-13 | P-13 | 96 |
| | 6-14 | P-14 | 96 |
| | 6-15 | P-15 | 95 |
| | 6-16 | P-16 | 94 |
| | 6-17 | P-17 | 96 |
| | 6-18 | P-18 | 94 |
| | 6-19 | P-19 | 95 |
| | 6-20 | P-20 | 94 |
| | 6-21 | P-21 | 95 |
| | 6-22 | P-22 | 96 |
| | 6-23 | P-23 | 95 |
| | 6-24 | P-24 | 94 |
| | 6-25 | P-25 | 95 |
| | 6-26 | P-26 | 96 |
| | 6-27 | P-27 | 95 |
| | 6-28 | P-28 | 96 |
| | 6-29 | P-29 | 95 |
| | 6-30 | P-30 | 96 |

TABLE 10

| | | | CHF₃/CF₄ gas etching |
|---|---|---|---|
| | | Polymer | rate (nm/min) |
| Comparative Example | 6-1 | CP-1 | 100 |
| | 6-2 | CP-2 | 99 |
| | 6-3 | CP-3 | 99 |
| | 6-4 | CP-4 | 98 |
| | 6-5 | CP-5 | 99 |
| | 6-6 | CP-6 | 98 |
| | 6-7 | CP-7 | 99 |
| | 6-8 | CP-8 | 97 |
| | 6-9 | CP-9 | 99 |
| | 6-10 | CP-10 | 98 |
| | 6-11 | CP-11 | 100 |
| | 6-12 | CP-12 | 101 |
| | 6-13 | CP-13 | 98 |
| | 6-14 | CP-14 | 98 |
| | 6-15 | CP-15 | 99 |
| | 6-16 | CP-16 | 97 |
| | 6-17 | CP-17 | 99 |
| | 6-18 | CP-18 | 98 |
| | 6-19 | CP-19 | 101 |
| | 6-20 | CP-20 | 98 |
| | 6-21 | CP-21 | 99 |
| | 6-22 | CP-22 | 98 |
| | 6-23 | CP-23 | 97 |
| | 6-24 | CP-24 | 98 |

It is evident from Tables 9 and 10 that the inventive polymers have good dry etch resistance, i.e., resistance to CHF₃/CF₄ gas etching.

Japanese Patent Application No. 2024-196586 is incorporated herein by reference.

Although some preferred embodiments have been described, many modifications and variations may be made thereto in light of the above teachings. It is therefore to be understood that the invention may be practiced otherwise than as specifically described without departing from the scope of the appended claims.

1. An onium salt monomer having the formula (A): 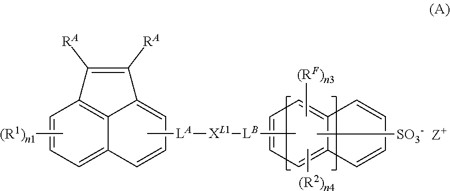
wherein n1 is 0, 1, 2, 3 or 4, n2 is 0 or 1, n3 is 0, 1, 2, 3 or 4, n4 is 0, 1, 2, 3 or 4, meeting 0≤n3+n4≤4 when n2=0 and 0≤n3+n4≤6 when n2=1,
R^{A} is each independently hydrogen, fluorine, methyl or trifluoromethyl,
R¹ is halogen, hydroxy, nitro, cyano, a C₁-C₂₀ hydrocarbyl group which may contain a heteroatom, a C₁-C₂₀ hydrocarbyloxy group which may contain a heteroatom, or a C₁-C₂₀ hydrocarbylthio group which may contain a heteroatom, when n1 is 2, 3 or 4, a plurality of R¹ may be identical or different and may bond together to form a ring with the carbon atom to which they are attached,
R² is halogen exclusive of fluorine, nitro, hydroxy, a C₁-C₂₀ hydrocarbyl group which may contain a heteroatom exclusive of fluorine, a C₁-C₂₀ hydrocarbyloxy group which may contain a heteroatom exclusive of fluorine, or a C₁-C₂₀ hydrocarbylthio group which may contain a heteroatom exclusive of fluorine; when n4 is 2, 3 or 4, a plurality of R² may be identical or different and may bond together to form a ring with the carbon atoms to which they are attached,
R^{F} is fluorine, a C₁-C₆ fluorinated saturated hydrocarbyl group, a C₁-C₆ fluorinated saturated hydrocarbyloxy group, or a C₁-C₆ fluorinated saturated hydrocarbylthio group, when n3 is 2, 3 or 4, a plurality of R^{F} may be identical or different,
L^{A} and L^{B} are each independently a single bond, ether bond, ester bond, amide bond, sulfonate ester bond, sulfonamide bond, carbonate bond or carbamate bond,
X^{L1} is a single bond or a C₁-C₄₀ hydrocarbylene group which may contain a heteroatom, and
Z⁺ is an onium cation.

2. The onium salt monomer of claim 1, having the formula (A1): 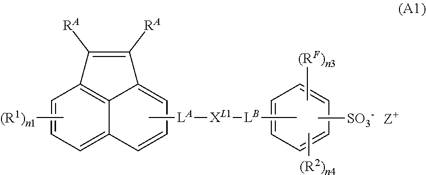
wherein n1, n3, n4, R^{A}, R¹, R², R^{F}, L^{A}, L^{B}, X^{L1}, and Z⁺ are as defined above.

3. The onium salt monomer of claim 2, having the formula (A2): 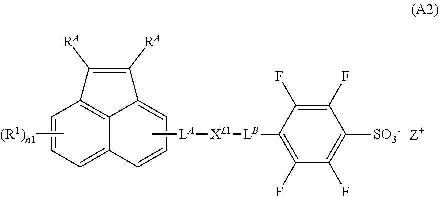
wherein n1, R^{A}, R¹, L^{A}, L^{B}, X^{L1}, and Z⁺ are as defined above.

4. The onium salt monomer of claim 1 wherein Z⁺ is a sulfonium cation having the formula (Z-1) or iodonium cation having the formula (Z-2): 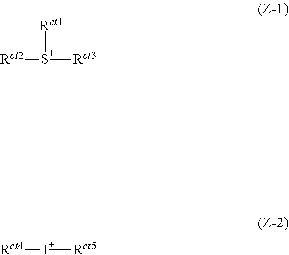
wherein R^{ct1} to R^{ct5} are each independently halogen or a C₁-C₃₀ hydrocarbyl group which may contain a heteroatom, and R^{ct1} and R^{ct2} may bond together to form a ring with the sulfur atom to which they are attached.

5. The onium salt of claim 1 wherein Z⁺ is a sulfonium cation having the formula (Z-3): 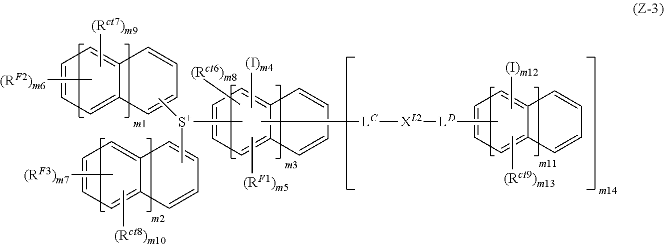
wherein m1 is 0 or 1, m2 is 0 or 1, m3 is 0 or 1, m4 is 0, 1, 2, 3 or 4, m5 is 0, 1, 2, 3 or 4, m6 is 0, 1, 2, 3, 4, 5 or 6, m7 is 0, 1, 2, 3, 4, 5 or 6, m8 is 0, 1 or 2, m9 is 0, 1 or 2, m10 is 0, 1 or 2, m11 is 0 or 1, m12 is 0, 1, 2, 3 or 4, m13 is 0, 1 or 2, m14 is 0, 1 or 2, meeting 0≤m6+m9≤4 when m1=0, 0≤m6+m9≤6 when m1=1, 0≤m7+m10≤4 when m2=0, 0≤m7+m10≤6 when m2=1, 1≤m4+m5+m8+m14≤4 when m3=0, 1≤m4+m5+m8+m14≤6 when m3=1, 0≤m12+m13≤4 when m11=0, 0≤m12+m13≤6 when m11=1, and m4+m12≥1,
R^{F1} to R^{F3} are each independently fluorine, a C₁-C₆ fluorinated saturated hydrocarbyl group, C₁-C₆ fluorinated saturated hydrocarbyloxy group, or C₁-C₆ fluorinated saturated hydrocarbylthio group, with the proviso that a plurality of R^{F1} may be identical or different when m5 is 2, 3 or 4, a plurality of R^{F2} may be identical or different when m6 is 2, 3, 4, 5 or 6, and a plurality of R^{F3} may be identical or different when m7 is 2, 3, 4, 5 or 6,
R^{ct6} to R^{ct9} are each independently halogen exclusive of iodine and fluorine, nitro, cyano, a C₁-C₂₀ hydrocarbyl group which may contain a heteroatom, C₁-C₂₀ hydrocarbyloxy group which may contain a heteroatom, or C₁-C₂₀ hydrocarbylthio group which may contain a heteroatom, with the proviso that two R^{ct6} may be identical or different and two R^{ct6} may bond together to form a ring with the carbon atoms to which they are attached when m8=2, two R^{ct7} may be identical or different and two R^{ct7} may bond together to form a ring with the carbon atoms to which they are attached when m9=2, two R^{ct8} may be identical or different and two R^{ct8} may bond together to form a ring with the carbon atoms to which they are attached when m10=2, two R^{ct9} may be identical or different and two R^{ct9} may bond together to form a ring with the carbon atoms to which they are attached when m13=2,
the aromatic rings directly bonded to S⁺ in the sulfonium cation may bond together to form a ring with S⁺,
L^{C} and L^{D} are each independently a single bond, ether bond, ester bond, sulfonate ester bond, amide bond, sulfonamide bond, carbonate bond or carbamate bond, and
X^{L2} is a single bond or a C₁-C₄₀ hydrocarbylene group which may contain a heteroatom.

6. A polymer comprising repeat units derived from the onium salt monomer of claim 1.

7. The polymer of claim 6 which functions as a polymer-bound acid generator.

8. The polymer of claim 6, further comprising repeat units of at least one type selected from repeat units having the formula (a1), repeat units having the formula (a2), and repeat units having the formula (a3): 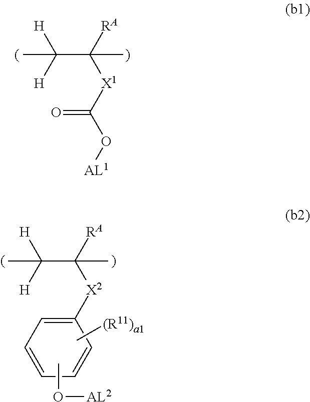
wherein R^{A} is each independently hydrogen, fluorine, methyl or trifluoromethyl,
X¹ is a single bond, phenylene group, naphthylene group, *—C(═O)—O—X¹¹—, or *—C(═O)—NH—X¹¹—, the phenylene and naphthylene groups may be substituted with hydroxy, nitro, cyano, a C₁-C₁₀ saturated hydrocarbyl moiety which may contain fluorine, C₁-C₁₀ saturated hydrocarbyloxy moiety which may contain fluorine, or halogen, X¹¹ is a C₁-C₁₀ saturated hydrocarbylene group, phenylene group or naphthylene group, the saturated hydrocarbylene group may contain hydroxy, ether bond, ester bond or lactone ring,
X² is a single bond, *—C(═O)—O—, or *—C(═O)—NH—,
* designates a point of attachment to the carbon atom in the backbone,
R¹¹ is halogen, cyano, hydroxy, nitro, a C₁-C₂₀ hydrocarbyl group which may contain a heteroatom, C₁-C₂₀ hydrocarbyloxy group which may contain a heteroatom, C₂-C₂₀ hydrocarbylcarbonyl group which may contain a heteroatom, C₂-C₂₀ hydrocarbylcarbonyloxy group which may contain a heteroatom, or C₂-C₂₀ hydrocarbyloxycarbonyl group which may contain a heteroatom, and when a1 is 2, 3 or 4, a plurality of R¹¹ may be identical or different,
AL¹ and AL² are each independently an acid labile group, and
a1 is 0, 1, 2, 3 or 4, 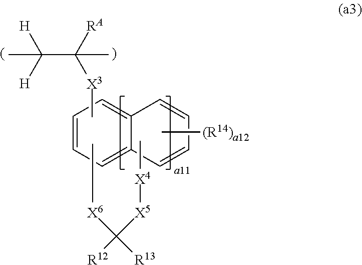
wherein all is 0 or 1, a12 is 0, 1, 2 or 3 when a11=0, a12 is 0, 1, 2, 3, 4 or 5 when a11=1,
R^{A} is hydrogen, fluorine, methyl or trifluoromethyl,
X³ is a single bond, *—C(═O)—O— or *—C(═O)—NH—, * designates a point of attachment to the carbon atom in the backbone,
X⁴ is a single bond, C₁-C₄ aliphatic hydrocarbylene group, carbonyl group, sulfonyl group or a combination thereof,
X⁵ and X⁶ are each independently oxygen or sulfur, with the proviso that X⁴ and X⁶ are attached to adjoining carbon atoms on the aromatic ring,
R¹² and R¹³ are each independently hydrogen or a C₁-C₂₀ hydrocarbyl group which may contain a heteroatom, R¹² and R¹³ may bond together to form a ring with the carbon atom to which they are attached,
R¹⁴ is halogen, hydroxy, cyano, nitro, a C₁-C₂₀ hydrocarbyl group which may contain a heteroatom, C₁-C₂₀ hydrocarbyloxy group which may contain a heteroatom, C₂-C₂₀ hydrocarbyloxycarbonyl group which may contain a heteroatom, C₁-C₂₀ hydrocarbylthio group which may contain a heteroatom, or —N(R^{14A})(R^{14B}), R^{14A} and R^{14B} are each independently hydrogen or a C₁-C₆ hydrocarbyl group, and when a12 is 2 or more, a plurality of R¹⁴ may be identical or different and a plurality of R¹⁴ may bond together to form a ring with the carbon atoms on the aromatic ring to which they are attached.

9. The polymer of claim 6, further comprising repeat units of at least one type selected from repeat units having the formula (b1) and repeat units having the formula (b2): 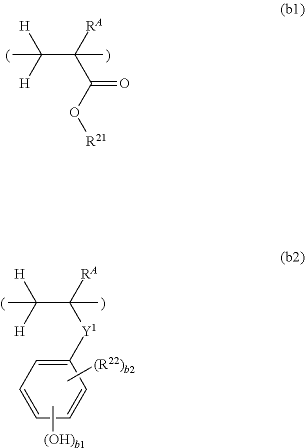
wherein R^{A} is each independently hydrogen, fluorine, methyl or trifluoromethyl,
Y¹ is a single bond or *—C(═O)—O—, * designates a point of attachment to the carbon atom in the backbone,
R²¹ is hydrogen or a C₁-C₂₀ group containing at least one structure selected from hydroxy other than phenolic hydroxy, cyano, carbonyl, carboxy, ether bond, ester bond, sulfonate ester bond, carbonate bond, lactone ring, sultone ring, and carboxylic anhydride (—C(═O)—O—C(═O)—),
R²² is halogen, carboxy, nitro, cyano, a C₁-C₂₀ hydrocarbyl group which may contain a heteroatom, C₁-C₂₀ hydrocarbyloxy group which may contain a heteroatom, C₂-C₂₀ hydrocarbylcarbonyl group which may contain a heteroatom, C₂-C₂₀ hydrocarbylcarbonyloxy group which may contain a heteroatom, or C₂-C₂₀ hydrocarbyloxycarbonyl group which may contain a heteroatom, and when b2 is 2, 3 or 4, a plurality of R²² may be identical or different,
b1 is 1, 2, 3 or 4, b2 is 0, 1, 2, 3 or 4, and 1≤b1+b2≤5.

10. A chemically amplified resist composition comprising (A) a base polymer containing the polymer of claim 6.

11. The resist composition of claim 10, further comprising (B) an organic solvent.

12. The resist composition of claim 10, further comprising (C) a quencher.

13. The resist composition of claim 10, further comprising (D) a photoacid generator.

14. The resist composition of claim 10, further comprising (E) a surfactant.

15. A pattern forming process comprising the steps of applying the chemically amplified resist composition of claim 10 onto a substrate to form a resist film thereon, exposing the resist film to high-energy radiation, and developing the exposed resist film in a developer.

16. The process of claim 15 wherein the high-energy radiation is KrF excimer laser, ArF excimer laser, EB or EUV of wavelength 3 to 15 nm.
